(19) United States
(12) Patent Application Publication
(10) Pub. No.: US 2023/0240141 A1
(43) Pub. Date: Jul. 27, 2023

(54) ORGANIC ELECTRONIC ELEMENT COMPRISING COMPOUND FOR ORGANIC ELECTRONIC ELEMENT, AND ELECTRONIC DEVICE THEREOF

(71) Applicant: DUK SAN NEOLUX CO., LTD., Cheonan-si, Chungcheongnam-do (KR)

(72) Inventors: Jong Gwang PARK, Cheonan-si, Chungcheongnam-do (KR); Nam Geol LEE, Cheonan-si, Chungcheongnam-do (KR); Sun Hee LEE, Cheonan-si, Chungcheongnam-do (KR); Soung Yun MUN, Cheonan-si, Chungcheongnam-do (KR); Jin Woo SHIN, Cheonan-si, Chungcheongnam-do (KR)

(73) Assignee: DUK SAN NEOLUX CO., LTD., Cheonan-si, Chungcheongnam-do (KR)

(21) Appl. No.: 17/998,210

(22) PCT Filed: May 12, 2021
(86) PCT No.: PCT/KR2021/005938

§ 371 Date: Nov. 8, 2022

(30) Foreign Application Priority Data
May 12, 2020 (KR) 10-2020-0056276

(51) Int. Cl. H10K 85/60; C07D 487/04; C09K 11/06; C07D 495/04; C07D 491/048

(52) U.S. Cl. CPC H10K 85/6572; C07D 487/04; C09K 11/06; C07D 495/04; H10K 85/657; C07D 491/048; H10K 50/11

(57) ABSTRACT

Provided are: an organic electronic element comprising an anode, a cathode, and an organic material layer between the anode and the cathode; and an electronic device comprising the organic electronic element, wherein the organic material layer comprises compounds represented by Formula 1 and Formula 1, respectively, and thus can lower the driving voltage of the organic electronic element and improve the luminosity and lifespan thereof.

### BACKGROUND

### Technical Field

The present invention relates to a compound for an organic electronic element, an organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function.

Lifespan and efficiency are the most problematic in organic electroluminescent device, and as displays become larger, these problems of efficiency and lifespan must be solved. Efficiency, lifespan, and driving voltage are related to each other, and when the efficiency is increased, the driving voltage is relatively decreased, and as the driving voltage is decreased, crystallization of the organic material due to Joule heating generated during driving decreases, and as a result, the lifespan tends to increase.

However, the efficiency cannot be maximized simply by improving the organic material layer. This is because, when the energy level and T1 value between each organic material layer, and the intrinsic properties of the material (mobility, interfacial properties, etc.) are optimally combined, a long lifespan and high efficiency can be achieved at the same time.

Also, in order to solve the problem of light emission in the hole transport layer in recent organic electroluminescent devices, an emitting-auxiliary layer must exist between the hole transport layer and the emitting layer, and it is time to develop different emitting-auxiliary layers according to each emitting layer (R, G, B).

In general, electrons are transferred from the electron transport layer to the emitting layer, and holes are transferred from the hole transport layer to the emitting layer, and excitons are generated by recombination.

However, since the material used for the hole transport layer should have a low HOMO value, most have a low T1 value. As a result, excitons generated in the emitting layer are transferred to the hole transport layer, resulting in charge unbalance in the emitting layer to emit light at the hole transport layer interface.

When light is emitted at the hole transport layer interface, the color purity and efficiency of the organic electronic element are lowered, and the lifespan is shortened. Therefore, it is urgently required to develop an emitting-auxiliary layer having a high T1 value and having a HOMO level between the HOMO energy level of the hole transport layer and the HOMO energy level of the emitting layer.

Furthermore, it is necessary to develop a hole injection layer material that delays the penetration and diffusion of metal oxides from the anode electrode (ITO) into the organic layer, which is one of the causes of shortening the lifespan of organic electronic element, and that has stable characteristics, that is, a high glass transition temperature, even against Joule heating generated during device driving. The low glass transition temperature of the hole transport layer material has a characteristic of lowering the uniformity of the thin film surface during device driving, which is reported to have a significant effect on device lifespan. Moreover, OLED devices are mainly formed by a deposition method, and it is necessary to develop a material that can withstand a long time during deposition, that is, a material with strong heat resistance.

In other words, in order to fully exhibit the excellent characteristics of an organic electronic element, it should be preceded that the material constituting the organic material layer in the device, such as a hole injection material, a hole transport material, a light emitting material, an electron transport material, an electron injection material, emitting auxiliary layer material, etc., is supported by a stable and efficient material, but the development of a stable and efficient organic material layer material for an organic electronic device has not yet been sufficiently made. Therefore, the development of new materials is continuously required.

As a reference prior art document, KR1020130076842 A was used.

### DETAILED DESCRIPTION OF THE INVENTION

### Summary

An object of the present invention is to provide an organic electronic element including a compound capable of lowering the driving voltage of the element and improving the luminous efficiency, color purity, stability and lifespan of the element, and an electronic device thereof.

### Technical Solution

In one aspect, the present invention provides an organic electronic element comprising an anode, a cathode, and an organic material layer formed between the anode and the cathode, wherein the organic material layer comprises an emitting layer, and a hole transport band formed between the emitting layer and the anode, wherein the hole transport band comprises a compound represented by Formula (1), and the emitting layer comprises an organic electronic element comprising a compound represented by Formula (2).

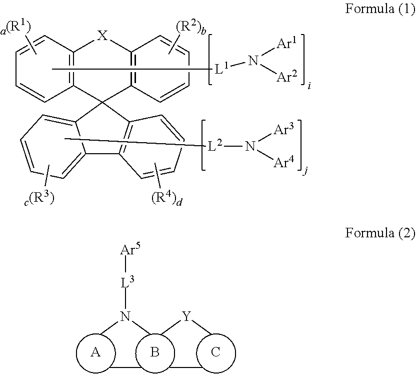

In another aspect, the present invention provides an electronic device including the organic electric element.

### Effects of the Invention

By using the compound according to the present invention, high luminous efficiency, low driving voltage and high heat resistance of the element can be achieved, and color purity and lifespan of the element can be greatly improved.

### BRIEF DESCRIPTION OF THE DRAWINGS

FIG. 1 to FIG. 3 are exemplary views of an organic electroluminescent device according to the present invention.

| | |
|---|---|
| 100, 200, 300: organic electronic element | 110: the first electrode |
| 120: hole injection layer | 130: hole transport layer |
| 140: emitting layer | 150: electron transport layer |
| 160: electron injection layer | 170: second electrode |
| 180: light efficiency enhancing Layer | 210: buffer layer |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

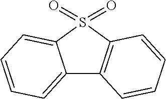 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

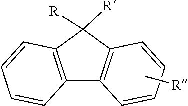

The term “spiro compound”, as used herein, has a ‘spiro union’, and a spiro union means a connection in which two rings share only one atom. At this time, atoms shared in the two rings are called ‘spiro atoms’, and these compounds are called ‘monospiro-’, ‘di-spiro-’ and ‘tri-spiro-’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and comprises a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

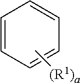

Here, when a is an integer of 0, the substituent R¹ is absent, when a is an integer of 1, the sole substituent R¹ is linked to any one of the carbon constituting the benzene ring, when a is an integer of 2 or 3, each is bonded as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

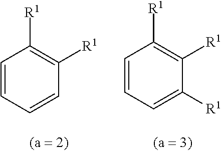

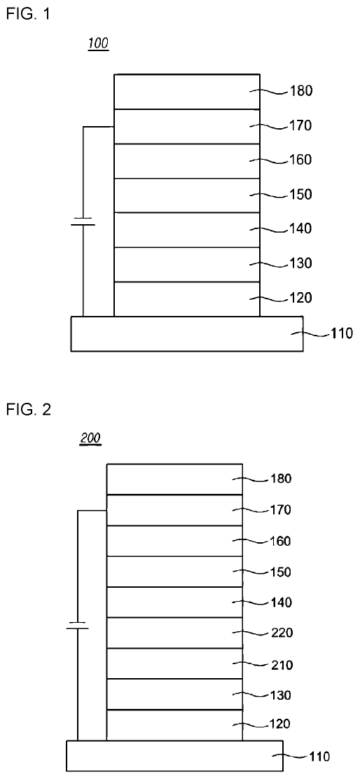

Hereinafter, the laminated structure of the organic electronic element comprising the compound of the present invention will be described with reference to FIG. 1 to 3.

In adding reference numerals to the components of each drawing, it should be noted that the same components are given the same reference numerals as much as possible even though they are indicated on different drawings. In addition, in describing the present invention, if it is determined that a detailed description of a related known configuration or function may obscure the gist of the present invention, the detailed description thereof will be omitted.

FIG. 1 to 3 are exemplary views of an organic electronic element according to an embodiment of the present invention.

Referring to FIG. 1, an organic electronic element (100) according to an embodiment of the present invention comprises a first electrode (110), a second electrode (170), and an organic material layer formed between the first electrode (110) and the second electrode (170) formed on a substrate (not shown).

The first electrode (110) may be an anode (anode), the second electrode (170) may be a cathode (cathode), and in the case of an inverted type, the first electrode may be a cathode and the second electrode may be an anode.

The organic material layer may include a hole injection layer (120), a hole transport layer (130), an emitting layer (140), an electron transport layer (150), and an electron injection layer (160). Specifically, the hole injection layer (120), the hole transport layer (130), the emitting layer (140), the electron transport layer (150), and the electron injection layer (160) may be sequentially formed on the first electrode (110).

Preferably, the light efficiency enhancing layer (180) may be formed on one surface of both surfaces of the first electrode (110) or the second electrode (170), not being contacted to the organic material layer, and when the light efficiency enhancing layer (180) is formed, the light efficiency of the organic electronic element may be improved.

For example, the light efficiency enhancing layer (180) may be formed on the second electrode (170), in the case of a top emission organic light emitting device, it is possible to reduce optical energy loss due to surface plasmon polaritons (SPPs) in the second electrode (170) by forming the light efficiency enhancing layer (180), and in the case of a bottom emission organic light emitting device, the light efficiency improving layer (180) may serve as a buffer for the second electrode (170).

A buffer layer (210) or an emitting auxiliary layer (220) may be further formed between the hole transport layer (130) and the emitting layer (140), which will be described with reference to FIG. 2.

Referring to FIG. 2, the organic electronic element (200) according to another embodiment of the present invention comprises a hole injection layer (120), a hole transport layer (130), a buffer layer (210), and an emitting auxiliary layer (220), an emitting layer (140), an electron transport layer (150), an electron injection layer (160) and a second electrode (170), sequentially formed on the first electrode (110), and a light efficiency enhancing layer (180) may be formed on the second electrode.

Although not shown in FIG. 2, an electron transport auxiliary layer may be further formed between the emitting layer (140) and the electron transport layer (150).

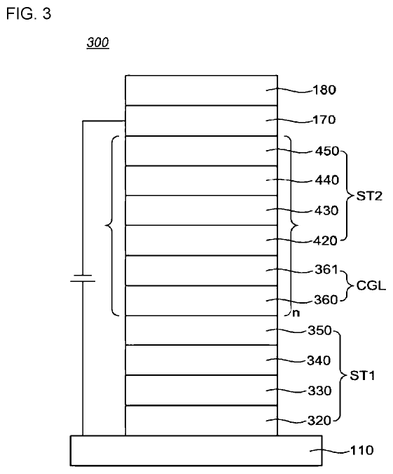

In addition, according to another embodiment of the present invention, the organic material layer may have a form in which a plurality of stacks including a hole transport layer, an emitting layer, and an electron transport layer are formed. This will be described with reference to FIG. 3.

Referring to FIG. 3, in the organic electronic element (300) according to another embodiment of the present invention, 2 or more sets of stacks (ST1, ST2) of an organic material layer comprising a multi-layered structure may be formed between the first electrode (110) and the second electrode (170), and a charge generation layer (CGL) may be formed between the stacks of the organic material layers.

Specifically, the organic electronic element according to an embodiment of the present invention may comprise a first electrode (110), a first stack (ST1), a charge generation layer (CGL), a second stack (ST2), and a second electrode (170) and the light efficiency enhancing layer (180).

The first stack (ST1), which is an organic material layer formed on the first electrode (110), may comprise a first hole injection layer (320), a first hole transport layer (330), a first emitting layer (340), and a first electron transport layer (350), and the second stack (ST2) may comprise a second hole injection layer (420), a second hole transport layer (430), a second emitting layer (440), and a second electron transport layer (450).

As such, the first stack and the second stack may be organic material layers having the same stacked structure or organic material layers having different stacked structures.

A charge generation layer (CGL) may be formed between the first stack (ST1) and the second stack (ST2). The charge generation layer (CGL) may comprise a first charge generation layer (360) and a second charge generation layer (361). The charge generation layer (CGL) is formed between the first emitting layer (340) and the second emitting layer (440) to increase the current efficiency generated in each emitting layer, and to smoothly distribute charges.

When a plurality of emitting layers are formed by a multi-layer stack structure method as shown in FIG. 3, an organic electroluminescent device emitting white light by the mixing effect of light emitted from each emitting layer may be manufactured, and an organic electroluminescent device emitting light of various colors may be manufactured.

The compound represented by Formula 1 of the present invention may be used as a material of the hole injection layer (120, 320, 420), the hole transport layer (130, 330, 430), the buffer layer (210), the emitting auxiliary layer (220), the electron transport layer (150, 350, 450), the electron injection layer (160), the emitting layer (140, 340, 440), or the light efficiency enhancing layer (180), but preferably, the compound represented by Formula 1 of the present invention may be used as a material for the hole transport band layer such as the hole transport layer (130, 330, 430) and/or the emitting auxiliary layer (220), and the compound represented by Formula 2 of the present invention may be used as a host of the emitting layers (140, 340, and 440).

Even with the same and similar core, the band gap, electrical properties, interface properties, etc. may vary depending on which position the substituent is bonded to, therefore it is necessary to study the selection of the core and the combination of sub-substituents bound thereto, and in particular, when the energy level and T1 value between each organic material layer, and the intrinsic properties (mobility, interfacial properties, etc.) of materials are optimally combined, long lifespan and high efficiency can be achieved at the same time.

The organic electroluminescent device according to an embodiment of the present invention may be manufactured using various deposition methods. It can be manufactured using a deposition method such as PVD or CVD, for example, by depositing a metal or a metal oxide having conductivity or an alloy thereof on a substrate to form the anode (110), and thereon, after forming an organic material layer including the hole injection layer (120), the hole transport layer (130), the emitting layer (140), the electron the transport layer (150) and the electron injection layer (160), it may be manufactured by depositing a material that can be used as the cathode (170) thereon. In addition, an emitting auxiliary layer (220) may be further formed between the hole transport layer (130) and the emitting layer (140), and an electron transport auxiliary layer (not shown) may be further formed between the emitting layer (140) and the electron transport layer (150), it can also be formed in a stack structure as shown.

Furthermore, the organic material layer may be manufactured in a smaller number of layers by a method such as a solution process or a solvent process, for example, a spin coating process, a nozzle printing process, an inkjet printing process, a slot coating process, a dip coating process, and a roll-to-roll process, doctor blading process, screen printing process, or a thermal transfer method, rather than a vapor deposition method, using various polymer materials. Since the organic material layer according to the present invention can be formed by various methods, the scope of the present invention is not limited by the formation method.

Also, the organic electronic element according to an embodiment of the present invention may be selected from the group consisting of an organic electroluminescent device, an organic solar cell, an organic photoreceptor, an organic transistor, a monochromatic lighting device, and a quantum dot display device.

Another embodiment of the present invention may include a display device including the organic electronic element of the present invention described above, and an electronic device including a control unit for driving the display device. In this case, the electronic device may be a current or future wired/wireless communication terminal, and includes all electronic devices such as a mobile communication terminal such as a mobile phone, a PDA, an electronic dictionary, a PMP, a remote control, a navigation system, a game machine, various TVs, and various computers.

Hereinafter, an organic electric device according to an aspect of the present invention will be described.

An organic electronic element according to an embodiment of the present invention comprises an anode, a cathode, and an organic material layer formed between the anode and the cathode, wherein the organic material layer comprises an emitting layer, and a hole transport band layer formed between the emitting layer and the anode, wherein the hole transport band layer comprises a compound represented by Formula 1, and the emitting layer comprises a compound represented by Formula 2.

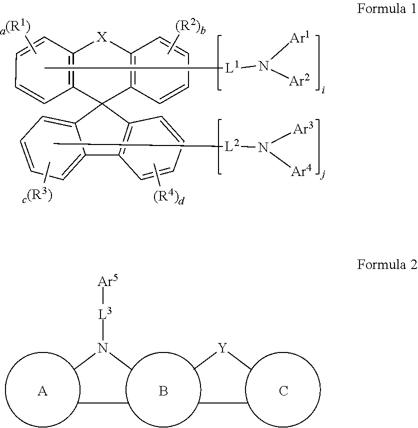

Wherein:

1) X is O, S or NR⁵.

2) Y is O, S or NR⁶.

3) Ring A, ring B and ring C are each independently a C₆-C₁₄ aryl group, also, Ring A may be substituted with R⁷, Ring B with R⁸, and Ring C may be substituted with R⁹.

4) R¹, R², R³, R⁴, R⁷, R⁸ and R⁹ are each independently the same as or different from each other, and are each independently selected from the group consisting of hydrogen; deuterium; halogen; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and C₆-C₆₀ arylamine group; or in case a, b, c and d are 2 or more, a plurality of adjacent R¹s, or a plurality of R²s, or a plurality of R³s, or a plurality of R⁴s may be bonded to each other to form a ring,

Wherein in case R¹, R², R³, R⁴, R⁷, R⁸ and R⁹ are an alkyl group, it may be preferably a C₁-C₃₀ alkyl group, and more preferably a C₁-C₂₄ alkyl group.

Wherein in case R¹, R², R³, R⁴, R⁷, R⁸ and R⁹ are an alkoxyl group, it may be preferably an C₁-C₂₄ alkoxyl group.

Wherein in case R¹, R², R³, R⁴, R⁷, R⁸ and R⁹ are an aryloxy group, it may be preferably an C₁-C₂₄ aryloxy group.

Wherein in case R¹, R², R³, R⁴, R⁷, R⁸ and R⁹ are an aryl group, it may be preferably a C₆-C₃₀ aryl group, and more preferably a C₆-C₂₄ aryl group, for example, it may be phenylene, biphenyl, naphthalene, terphenyl, etc.

Wherein in case R¹, R², R³, R⁴, R⁷, R⁸ and R⁹ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

Wherein in case R¹, R², R³, R⁴, R⁷, R⁸ and R⁹ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case R¹, R², R³, R⁴, R⁷, R⁸ and R⁹ are an arylamine group, it may be preferably a C₆-C₃₀ arylamine group, and more preferably a C₆-C₂₄ arylamine group,

5) R⁵ is an C₆˜C₆₀ aryl group; or a C₂-C₆₀ heterocyclic group including at least one heteroatom of O, N, S, Si or P;

R⁶ is an C₆˜C₆₀ aryl group; or a C₂-C₆₀ heterocyclic group including at least one heteroatom of O, N, S, Si or P; or L-Ar; L is same as L¹, Ar is same as Ar¹,

Wherein in case R⁵ and R⁶ are an aryl group, it may be preferably a C₆-C₃₀ aryl group, and more preferably a C₆-C₂₄ aryl group, for example, it may be phenylene, biphenyl, naphthyl, phenanthrene, terphenyl, etc.

Wherein in case R⁵ and R⁶ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, triazine or quinoxaline.

6) a, b, c and d are each independently an integer of 0 to 4.

7) i and j are each independently an integer of 0 to 2, provided that i+j is an integer of 1 or more.

8) L¹, L² and L³ are each independently selected from the group consisting of a single bond; a C₆-C₆₀ arylene group; fluorenylene group; a fused ring group of a C₃-C₆₀ aliphatic ring and a C₆-C₆₀ aromatic ring; a C₂-C₆₀ heterocyclic group;

Wherein in case L¹, L² and L³ are an arylene group, it may be preferably a C₆-C₃₀ arylene group, more preferably a C₆-C₂₄ arylene group, for example, phenylene, biphenyl, naphthalene, terphenyl, etc.

Wherein in case L¹, L² and L³ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

Wherein in case L¹, L² and L³ are a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

9) Ar¹, Ar², Ar³, Ar⁴ and Ar⁵ are each independently selected from the group consisting of a C₁-C₆ alkyl group; a C₂-C₂₀ alkenyl group; a C₂-C₂₀ alkynyl group; a C₁-C₃₀ alkoxyl group; a C₆-C₃₀ aryloxy group; a C₆-C₆₀ aryl group; fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring; alternatively, Ar¹ and Ar² or Ar³ and Ar⁴ may be bonded to each other to form a ring.

Wherein in case Ar¹, Ar², Ar³, Ar⁴ and Ar⁵ are an alkyl group, it may be preferably a C₁-C₃₀ alkyl group, and more preferably a C₁-C₂₄ alkyl group.

Wherein in case Ar¹, Ar², Ar³, Ar⁴ and Ar⁵ are an alkoxyl group, it may be preferably an C₁˜C₂₄ alkoxyl group.

Wherein in case Ar¹, Ar², Ar³, Ar⁴ and Ar⁵ are an aryloxy group, it may be preferably an C₁˜C₂₄ aryloxy group.

In case Ar¹, Ar², Ar³, Ar⁴ and Ar⁵ are an aryl group, it may be preferably a C₆-C₃₀ aryl group, and more preferably a C₆-C₂₄ aryl group, for example, it may be phenylene, biphenyl, naphthalene, terphenyl, etc.

In case Ar¹, Ar², Ar³, Ar⁴ and Ar⁵ are an a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, benzothienopyrimidine, benzofuropyrimidine, phenothiazine, phenylphenothiazine, etc.

In case Ar¹, Ar², Ar³, Ar⁴ and Ar⁵ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

10) wherein the aryl group, arylene group, arylamine group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group; and C₈-C₂₀ arylalkenyl group; and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, the compound represented by Formula 1 is represented by any one of Formulas 1-1 to 1-7

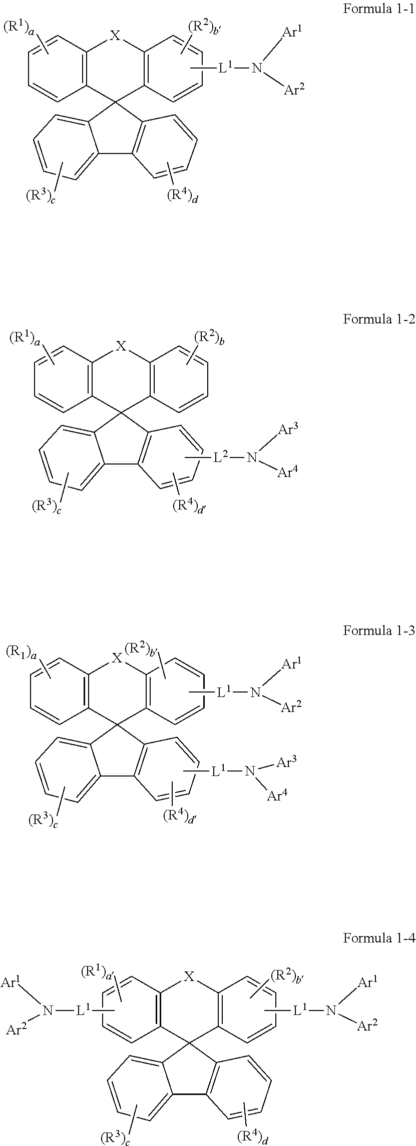

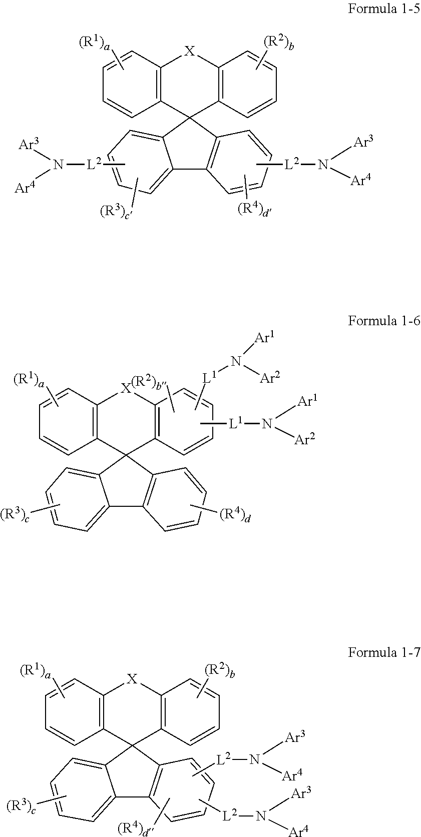

Wherein,

1) X, R¹, R², R³, R⁴, a, b, c, d, L¹, L², Ar¹, Ar², Ar³ and Ar⁴ are the same as define above,

2) a′, b′, c′ and d′ are each independently an integer of 0 to 3,

3) b″ and d″ are each independently an integer of 0 to 2.

Also, the compound represented by Formula 1 is represented by any one of Formulas 1-8 to 1-10.

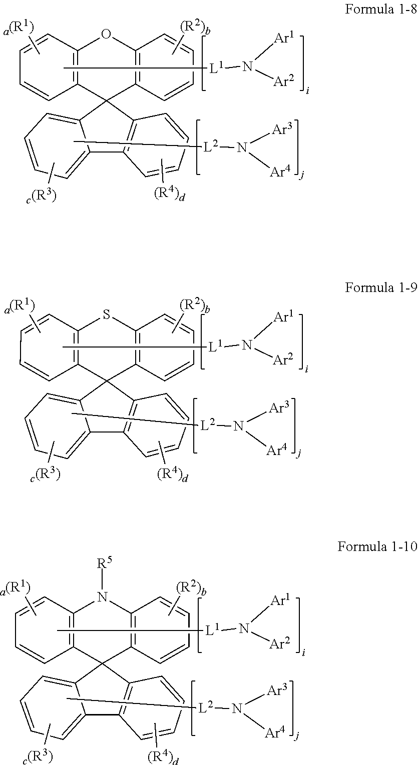

Wherein, R¹, R², R³, R⁴, a, b, c, d, L¹, L², Ar¹, Ar², Ar³, Ar⁴, i and j are the same as above.

Also, at least one of Ar¹ to Ar⁴ in Formula 1 is represented by Formula B-1.

Formula B-1

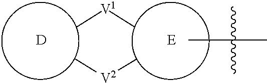

Wherein,

1) V¹ and V² are each independently a single bond, NR¹⁰, CR¹¹R¹², O or S,

2) R¹⁰, R¹¹ and R¹² are the same as the definition of R⁵, or R¹¹ and R¹² may be bonded to each other to form a ring,

3) Ring D and Ring E are each independently a C₆-C₂₀ aryl group; or C₄˜C₂₀ heterocyclic group;

Also, in any one of R¹ to R⁴ in Formula 1, an adjacent pair is bonded to each other to form any one of benzene, indole, indene, benzofuran, and benzothiophene.

Specifically, the compound represented by Formula 1 may be any one of the following compounds.

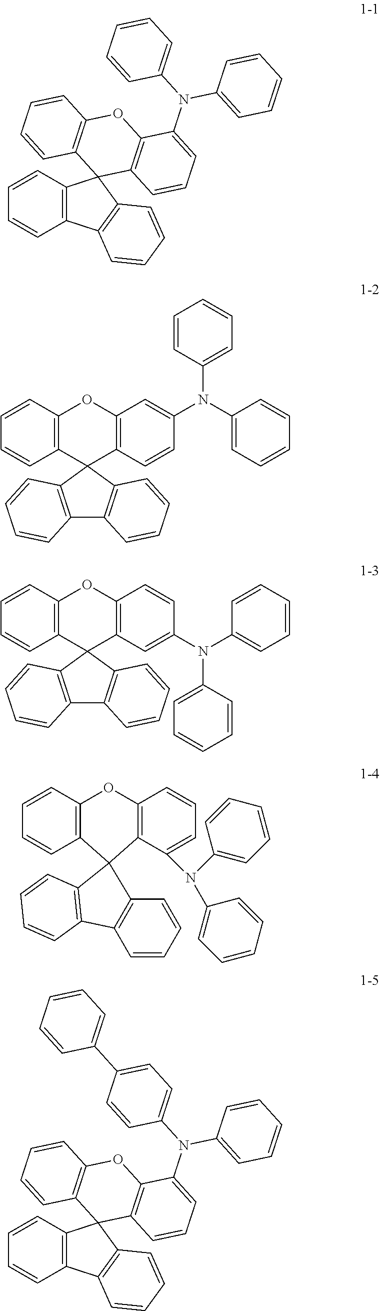

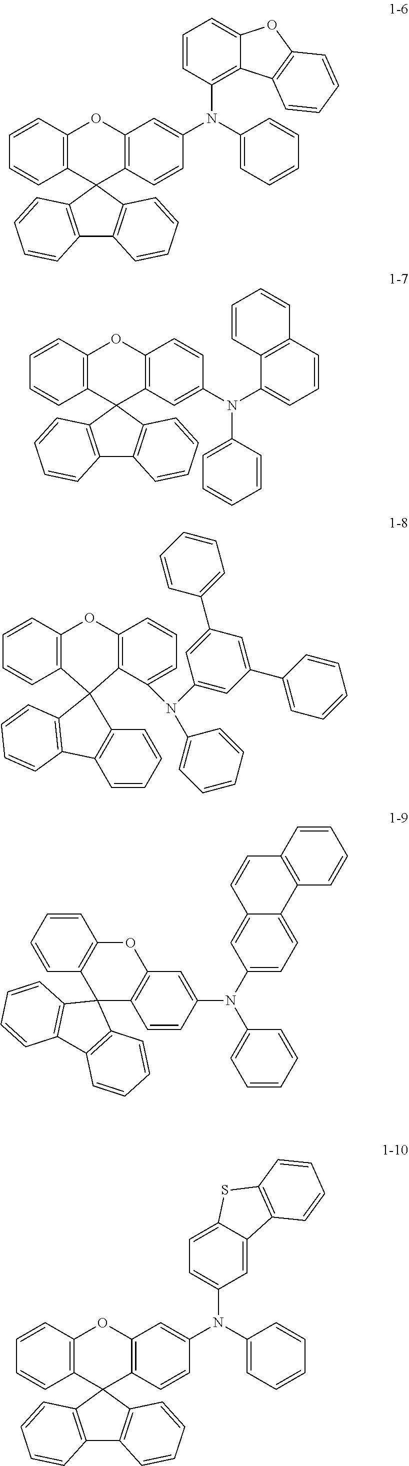

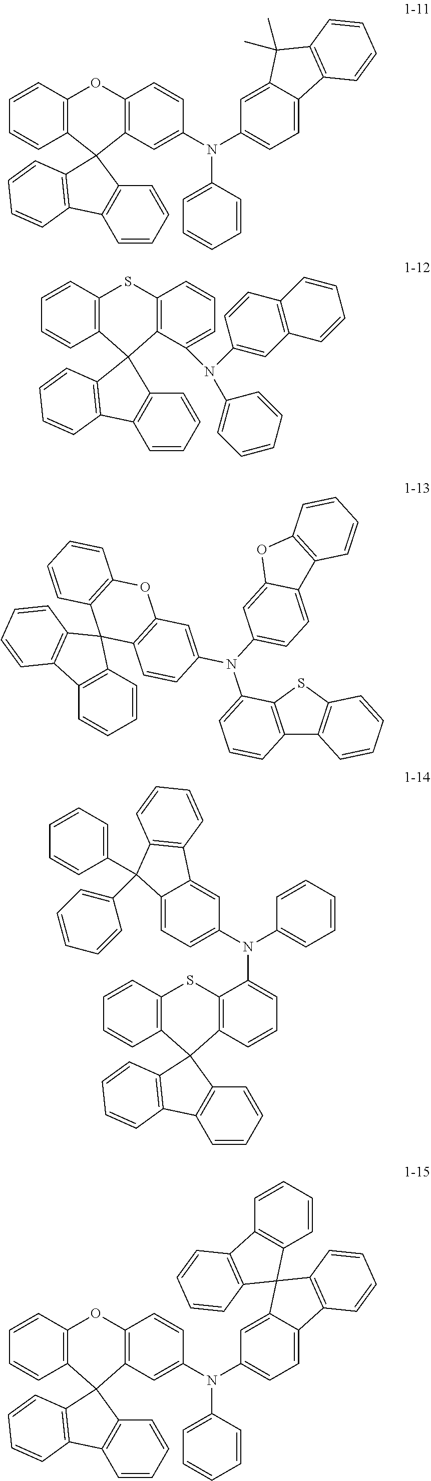

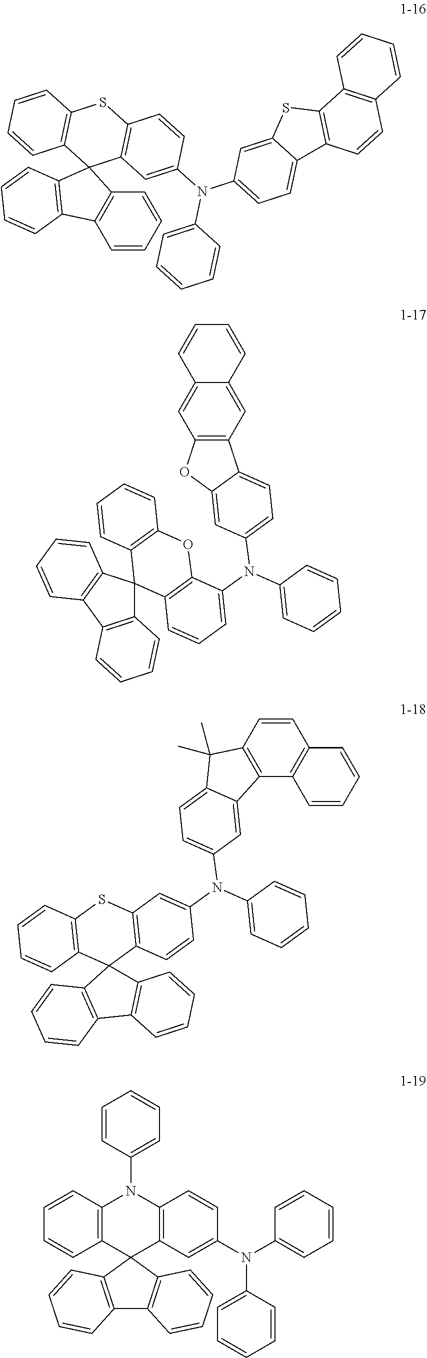

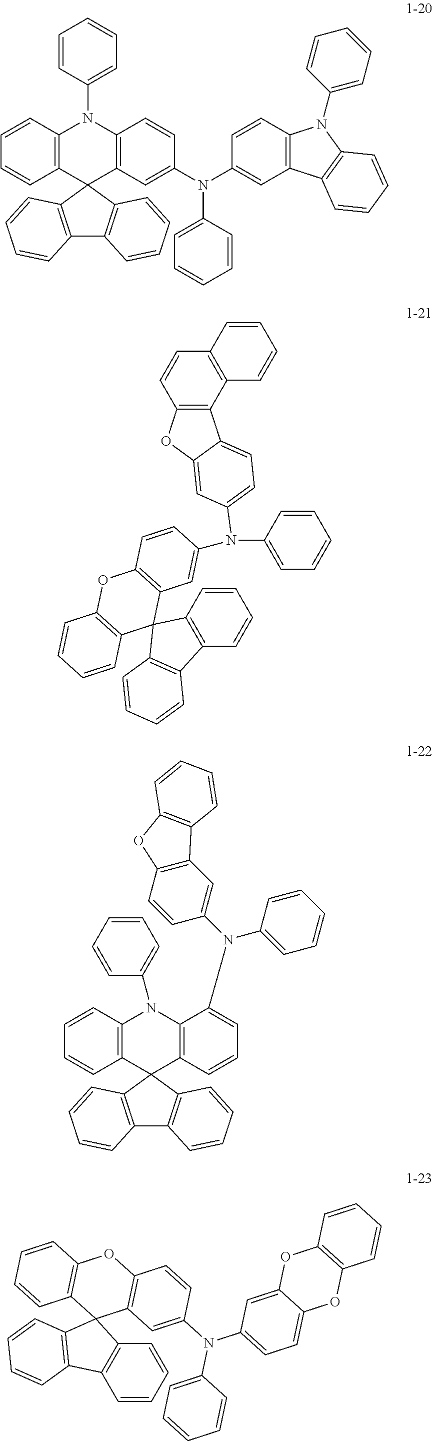

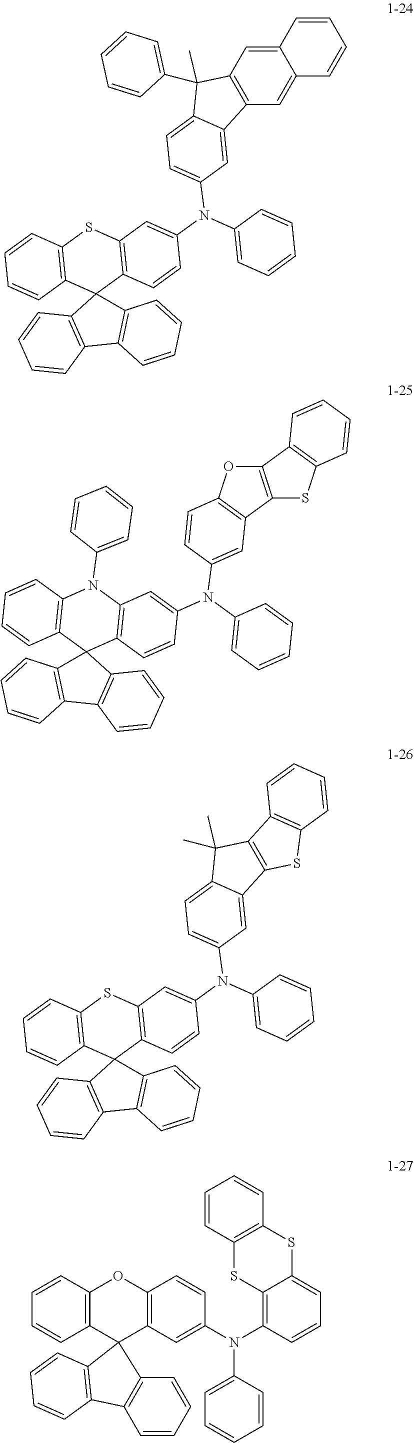 [SMILES: CC1(c2ccccc2)c2ccc(N(c3ccccc3)c3ccc4c(c3)Sc3ccccc3C43c4ccccc4-c4ccccc43)cc2-c2cc3ccccc3cc21 ; c1ccc(N(c2ccc3c(c2)N(c2ccccc2)c2ccccc2C32c3ccccc3-c3ccccc32)c2ccc3oc4c5ccccc5sc4c3c2)cc1 ; CC1(C)c2ccc(N(c3ccccc3)c3ccc4c(c3)Sc3ccccc3C43c4ccccc4-c4ccccc43)cc2-c2sc3ccccc3c21 ; c1ccc(N(c2ccc3c(c2)C2(c4ccccc4O3)c3ccccc3-c3ccccc32)c2cccc3c2Sc2ccccc2S3)cc1]

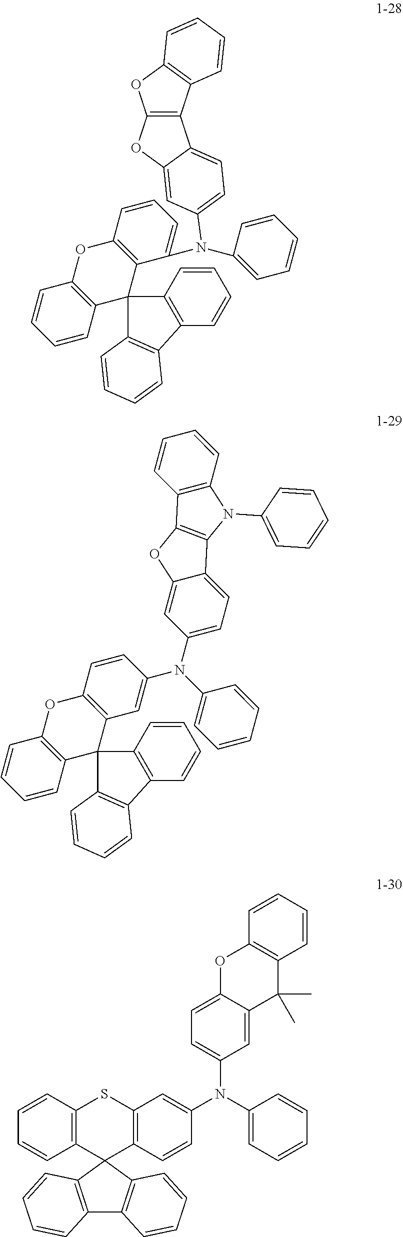

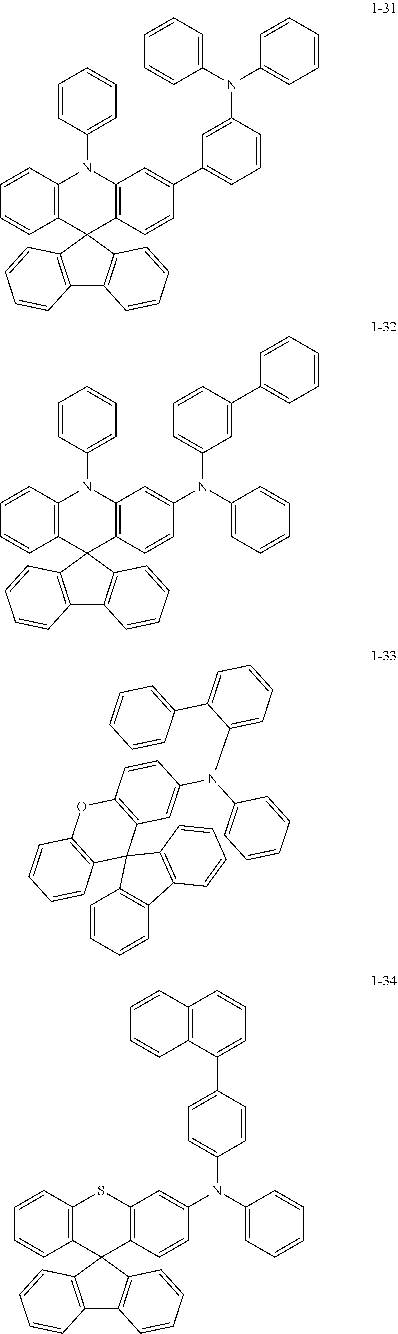 [SMILES: c1ccc(N(c2ccccc2)c2cccc(-c3ccc4c(c3)N(c3ccccc3)c3ccccc3C43c4ccccc4-c4ccccc43)c2)cc1 ; c1ccc(-c2cccc(N(c3ccccc3)c3ccc4c(c3)N(c3ccccc3)c3ccccc3C43c4ccccc4-c4ccccc43)c2)cc1 ; c1ccc(-c2ccccc2N(c2ccccc2)c2ccc3c(c2)C2(c4ccccc4O3)c3ccccc3-c3ccccc32)cc1 ; c1ccc(N(c2ccc(-c3cccc4ccccc34)cc2)c2ccc3c(c2)Sc2ccccc2C32c3ccccc3-c3ccccc32)cc1]

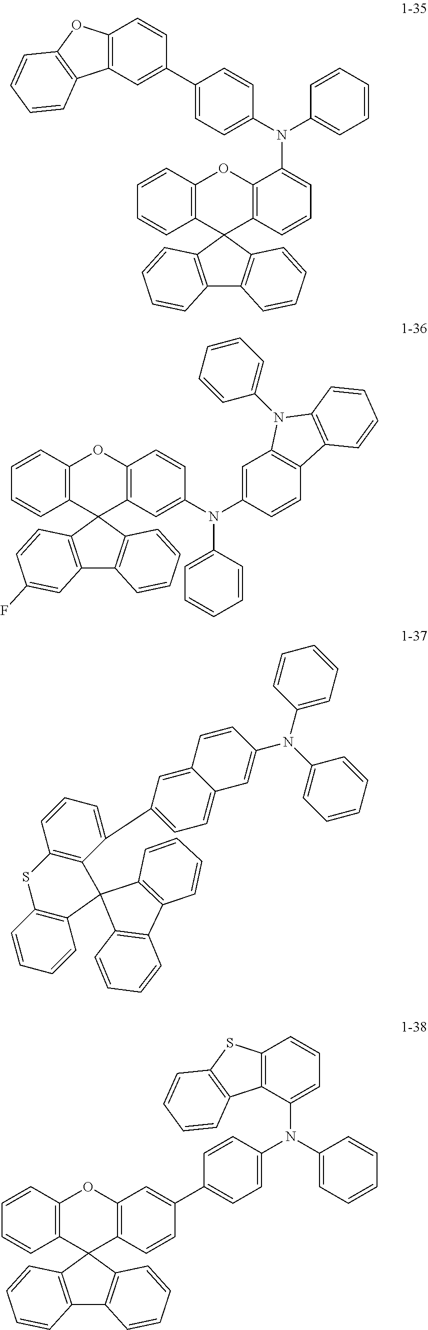

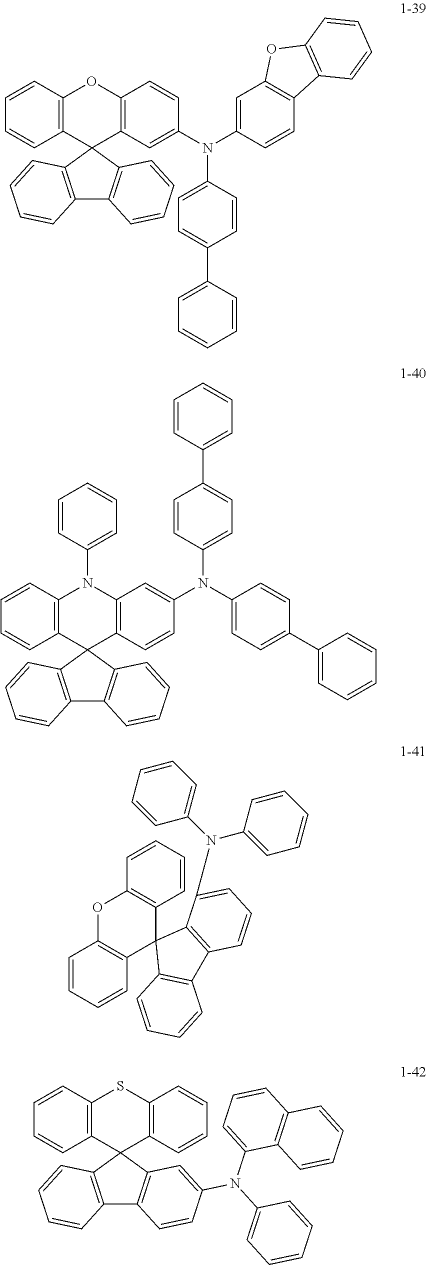

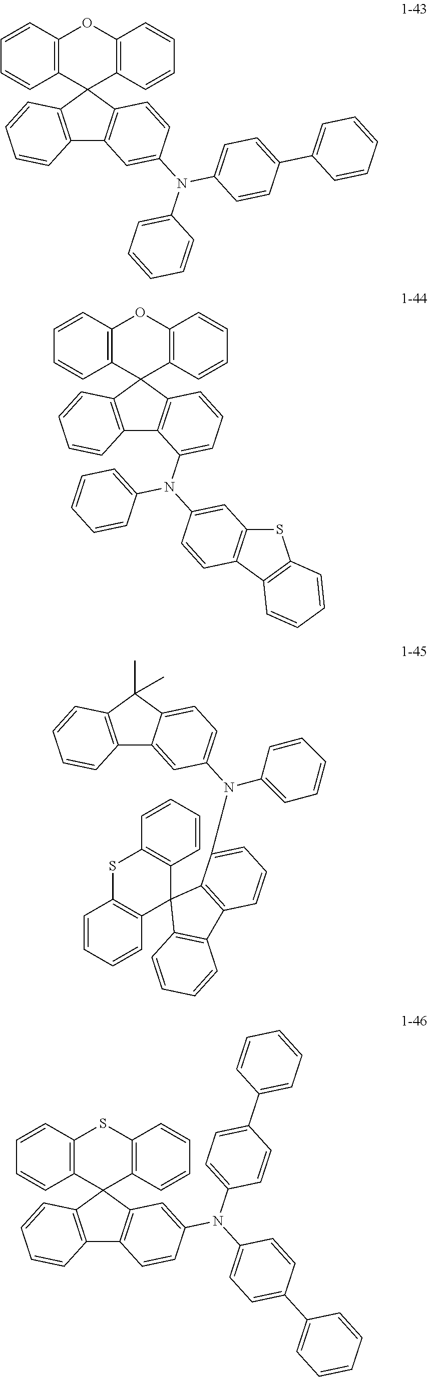

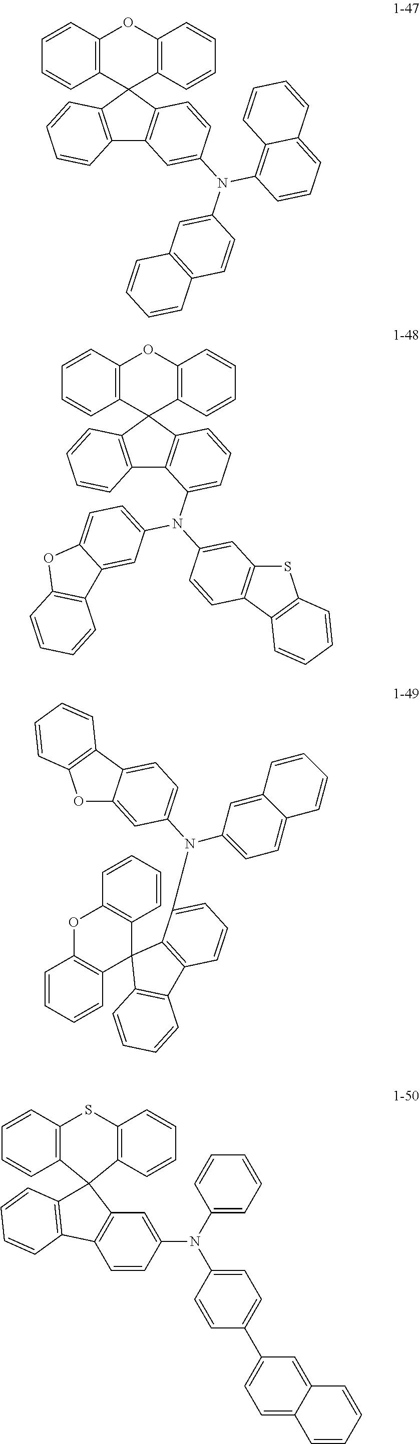

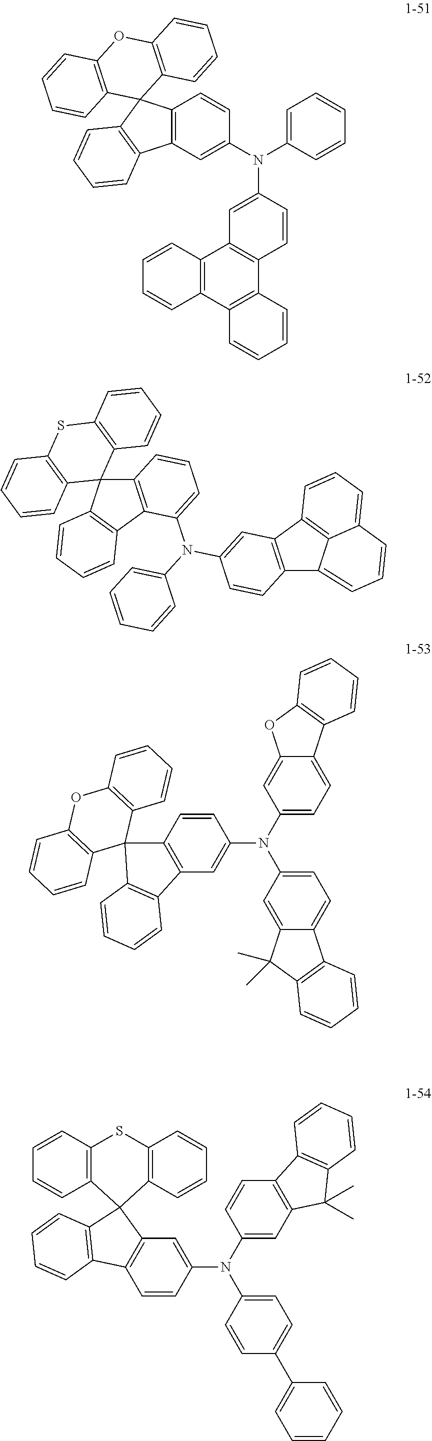 [SMILES: c1ccc(N(c2ccc3c(c2)-c2ccccc2C32c3ccccc3Oc3ccccc32)c2ccc3c4ccccc4c4ccccc4c3c2)cc1 ; c1ccc(N(c2ccc3c(c2)-c2cccc4cccc-3c24)c2cccc3c2-c2ccccc2C32c3ccccc3Sc3ccccc32)cc1 ; CC1(C)c2ccccc2-c2ccc(N(c3ccc4c(c3)-c3ccccc3C43c4ccccc4Oc4ccccc43)c3ccc4c(c3)oc3ccccc34)cc21 ; CC1(C)c2ccccc2-c2ccc(N(c3ccc(-c4ccccc4)cc3)c3ccc4c(c3)C3(c5ccccc5Sc5ccccc53)c3ccccc3-4)cc21]

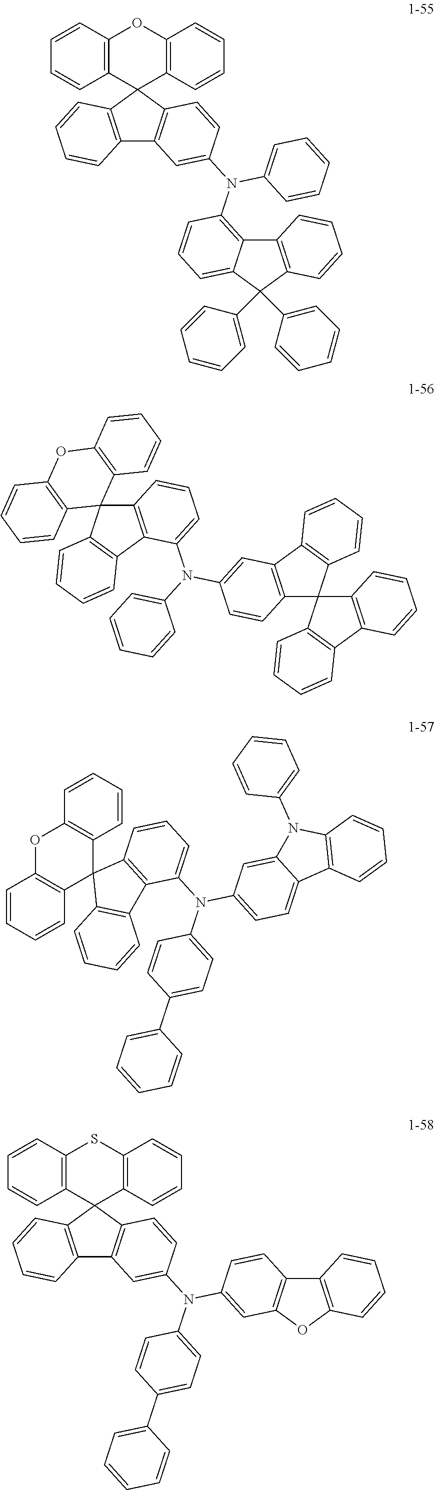 [SMILES: c1ccc(N(c2ccc3c(c2)-c2ccccc2C32c3ccccc3Oc3ccccc32)c2cccc3c2-c2ccccc2C3(c2ccccc2)c2ccccc2)cc1 ; c1ccc(N(c2ccc3c(c2)-c2ccccc2C32c3ccccc3-c3ccccc32)c2cccc3c2-c2ccccc2C32c3ccccc3Oc3ccccc32)cc1 ; c1ccc(-c2ccc(N(c3ccc4c5ccccc5n(-c5ccccc5)c4c3)c3cccc4c3-c3ccccc3C43c4ccccc4Oc4ccccc43)cc2)cc1 ; c1ccc(-c2ccc(N(c3ccc4c(c3)-c3ccccc3C43c4ccccc4Sc4ccccc43)c3ccc4c(c3)oc3ccccc34)cc2)cc1]

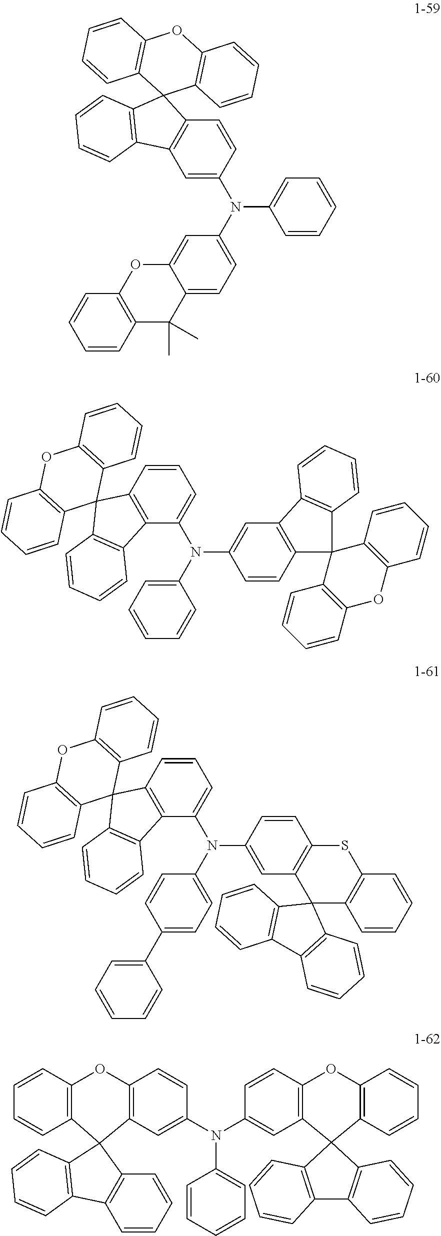 [SMILES: CC1(C)c2ccccc2Oc2cc(N(c3ccccc3)c3ccc4c(c3)-c3ccccc3C43c4ccccc4Oc4ccccc43)ccc21 ; c1ccc(N(c2ccc3c(c2)-c2ccccc2C32c3ccccc3Oc3ccccc32)c2cccc3c2-c2ccccc2C32c3ccccc3Oc3ccccc32)cc1 ; c1ccc(-c2ccc(N(c3ccc4c(c3)C3(c5ccccc5S4)c4ccccc4-c4ccccc43)c3cccc4c3-c3ccccc3C43c4ccccc4Oc4ccccc43)cc2)cc1 ; c1ccc(N(c2ccc3c(c2)C2(c4ccccc4O3)c3ccccc3-c3ccccc32)c2ccc3c(c2)C2(c4ccccc4O3)c3ccccc3-c3ccccc32)cc1]

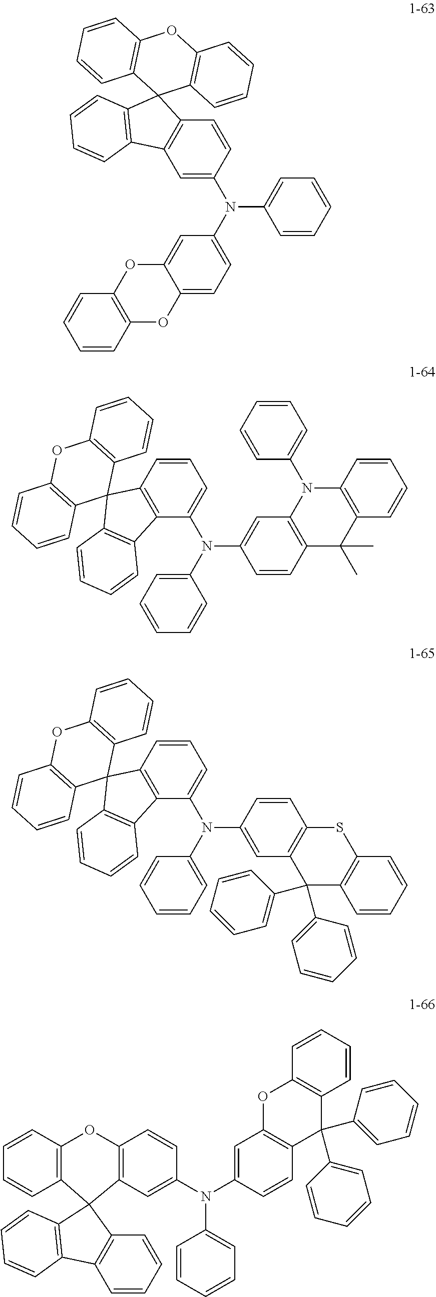 [SMILES: c1ccc(N(c2ccc3c(c2)Oc2ccccc2O3)c2ccc3c(c2)-c2ccccc2C32c3ccccc3Oc3ccccc32)cc1 ; CC1(C)c2ccccc2N(c2ccccc2)c2cc(N(c3ccccc3)c3cccc4c3-c3ccccc3C43c4ccccc4Oc4ccccc43)ccc21 ; c1ccc(N(c2ccc3c(c2)C(c2ccccc2)(c2ccccc2)c2ccccc2S3)c2cccc3c2-c2ccccc2C32c3ccccc3Oc3ccccc32)cc1 ; c1ccc(N(c2ccc3c(c2)Oc2ccccc2C3(c2ccccc2)c2ccccc2)c2ccc3c(c2)C2(c4ccccc4O3)c3ccccc3-c3ccccc32)cc1]

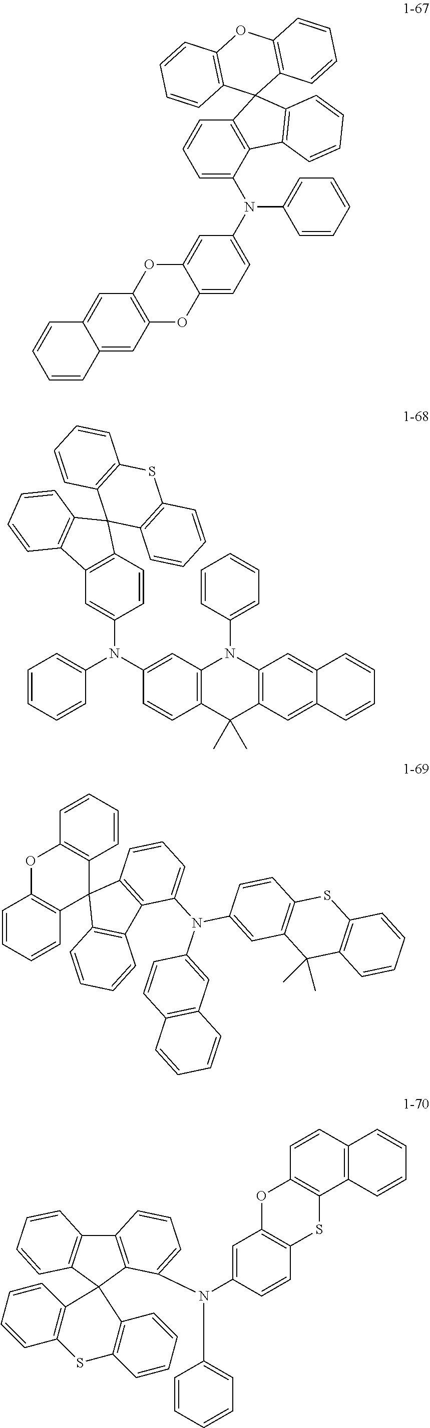

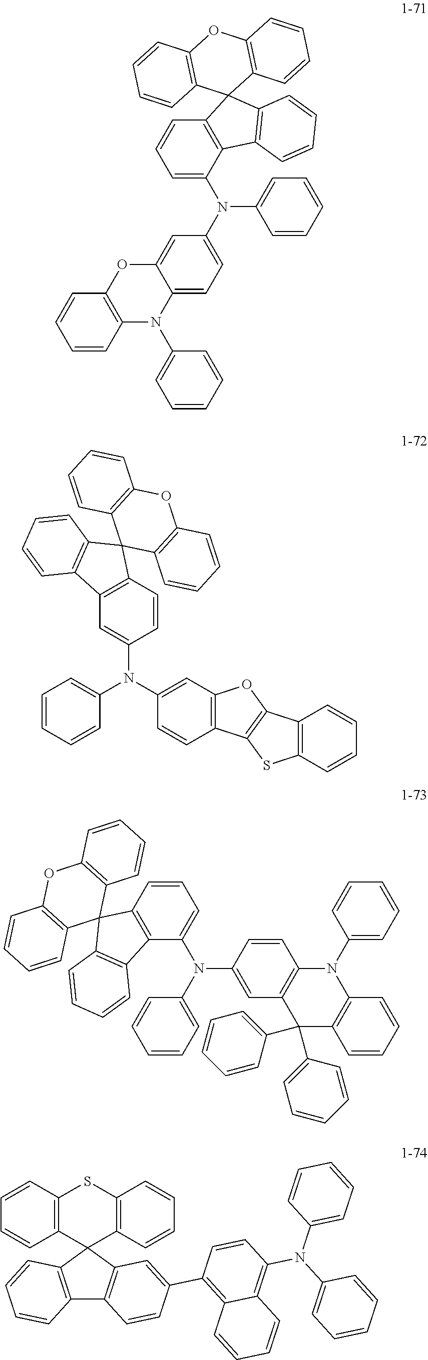

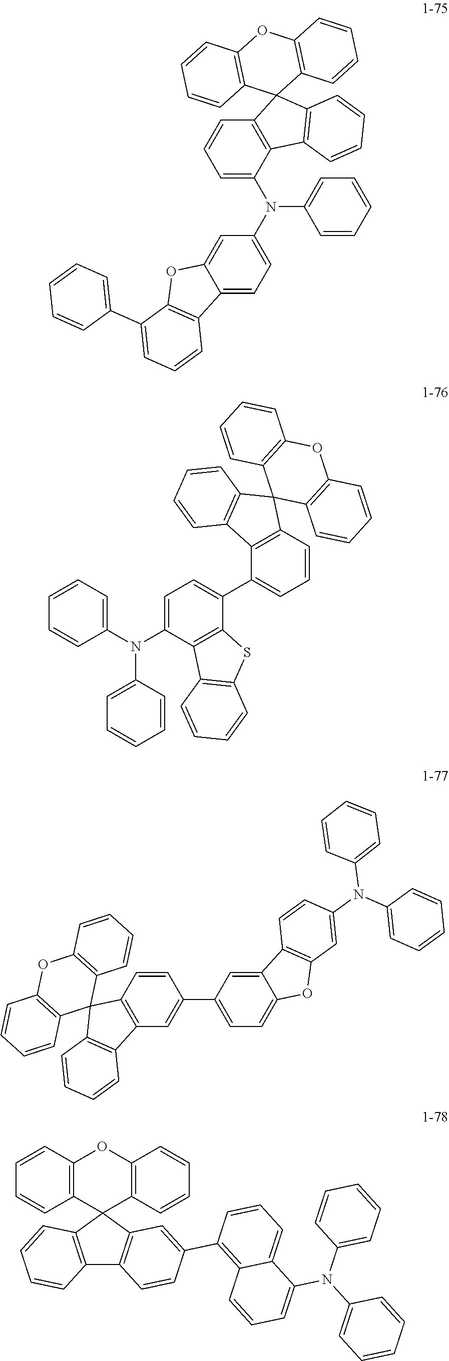 [SMILES: c1ccc(-c2cccc3c2oc2cc(N(c4ccccc4)c4cccc5c4-c4ccccc4C54c5ccccc5Oc5ccccc54)ccc23)cc1 ; c1ccc(N(c2ccccc2)c2ccc(-c3cccc4c3-c3ccccc3C43c4ccccc4Oc4ccccc43)c3sc4ccccc4c23)cc1 ; c1ccc(N(c2ccccc2)c2ccc3c(c2)oc2ccc(-c4ccc5c(c4)-c4ccccc4C54c5ccccc5Oc5ccccc54)cc23)cc1 ; c1ccc(N(c2ccccc2)c2cccc3c(-c4ccc5c(c4)C4(c6ccccc6Oc6ccccc64)c4ccccc4-5)cccc23)cc1]

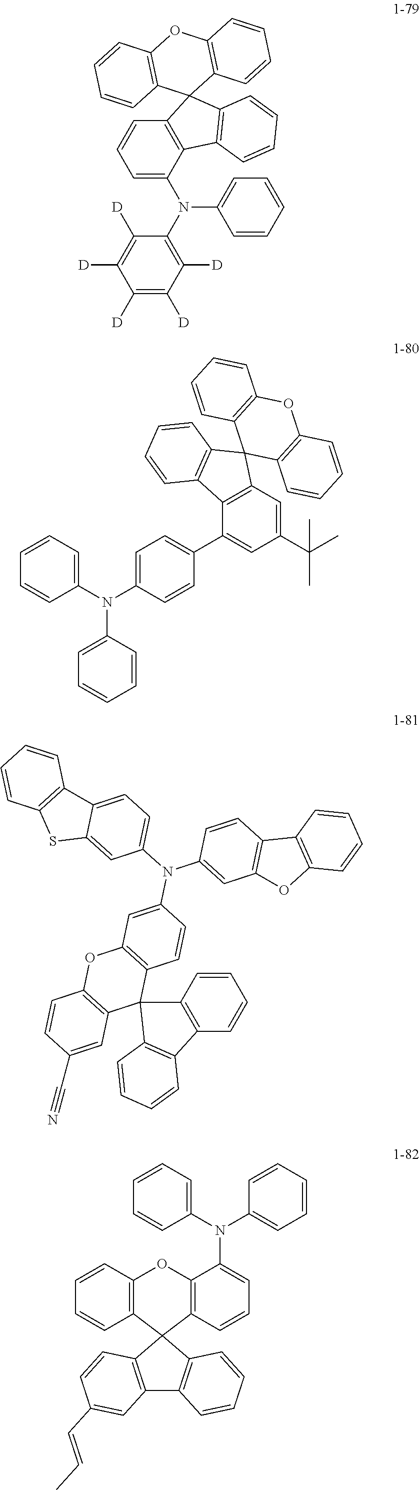

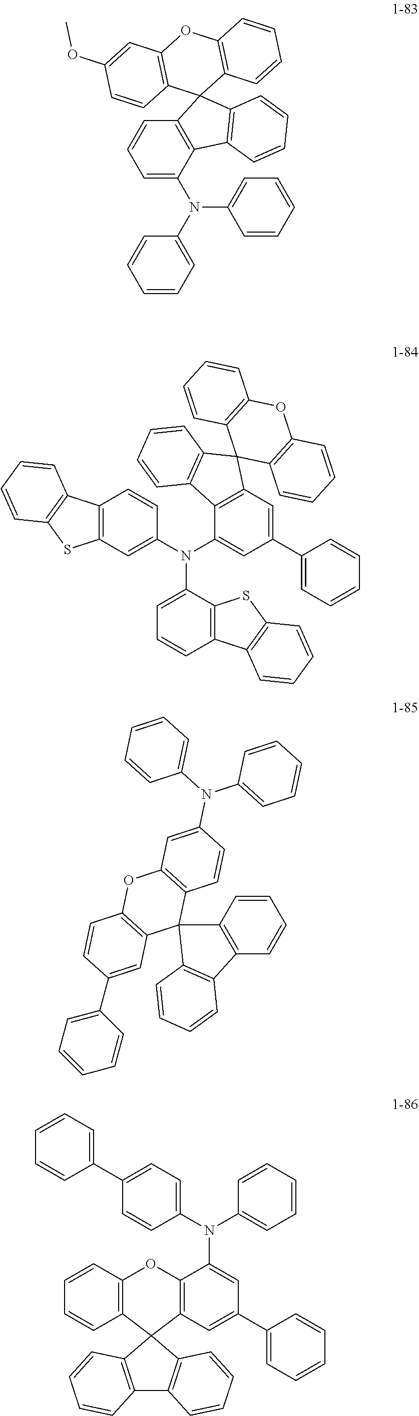

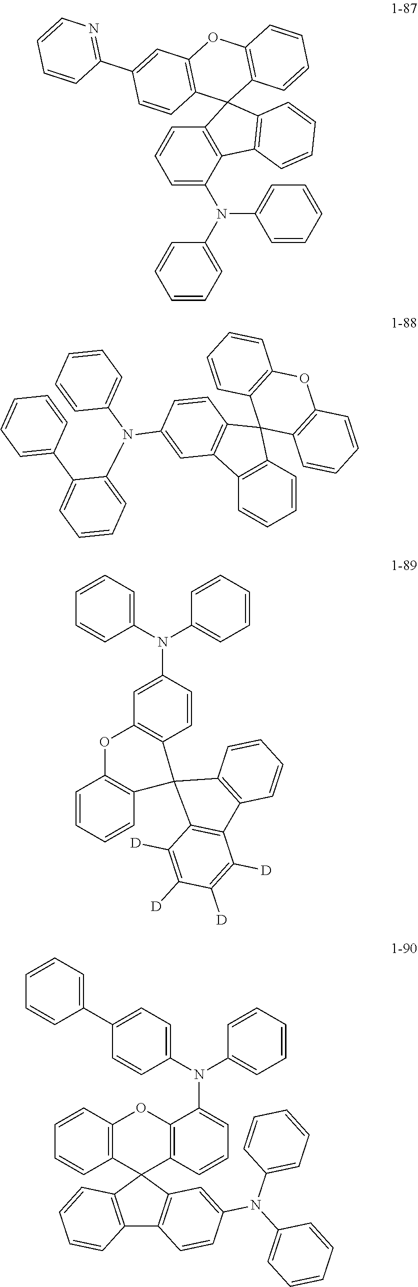

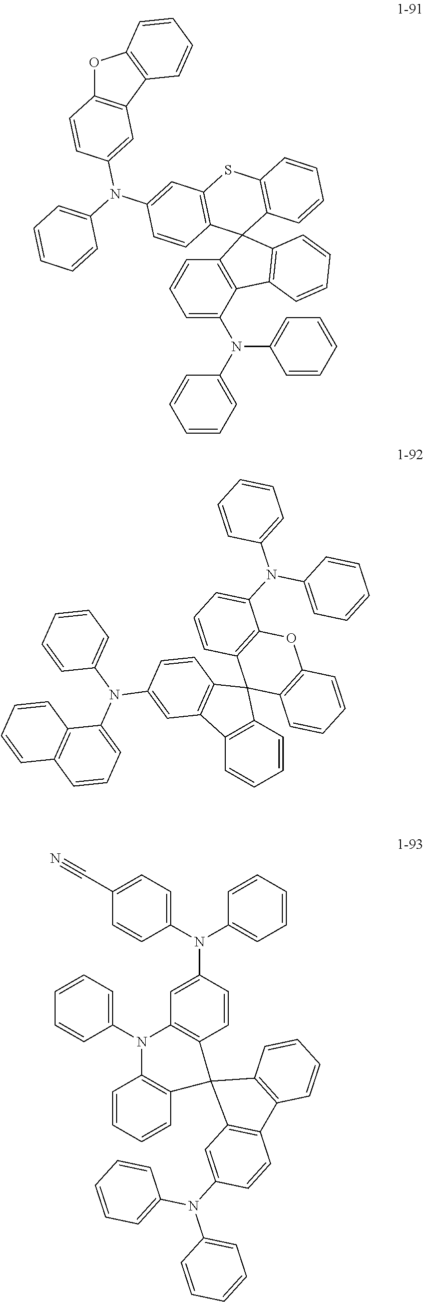 [SMILES: c1ccc(N(c2ccc3c(c2)Sc2ccccc2C32c3ccccc3-c3c(N(c4ccccc4)c4ccccc4)cccc32)c2ccc3oc4ccccc4c3c2)cc1 ; c1ccc(N(c2ccccc2)c2cccc3c2Oc2ccccc2C32c3ccccc3-c3cc(N(c4ccccc4)c4cccc5ccccc45)ccc32)cc1 ; N#Cc1ccc(N(c2ccccc2)c2ccc3c(c2)N(c2ccccc2)c2ccccc2C32c3ccccc3-c3ccc(N(c4ccccc4)c4ccccc4)cc32)cc1]

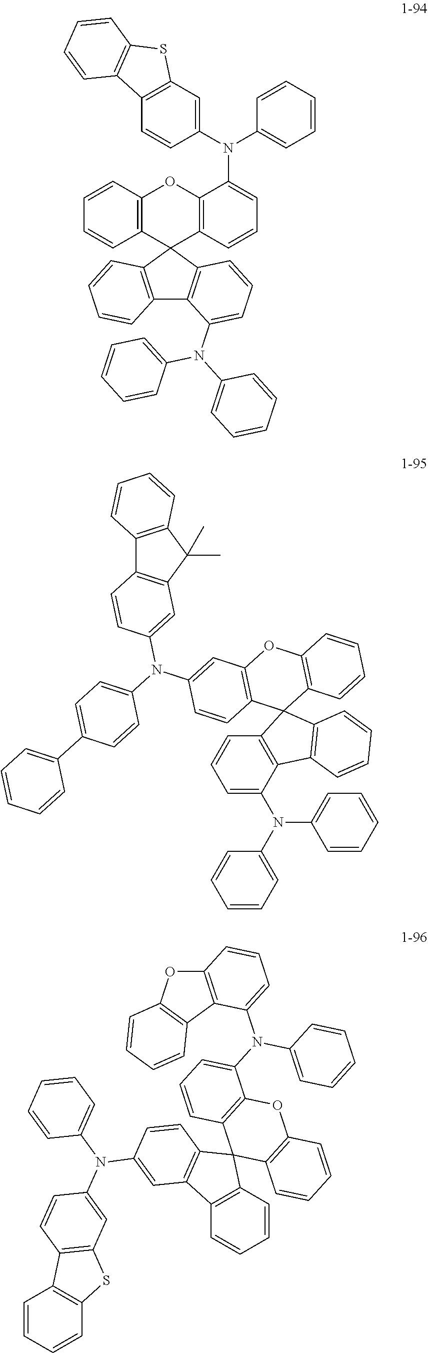

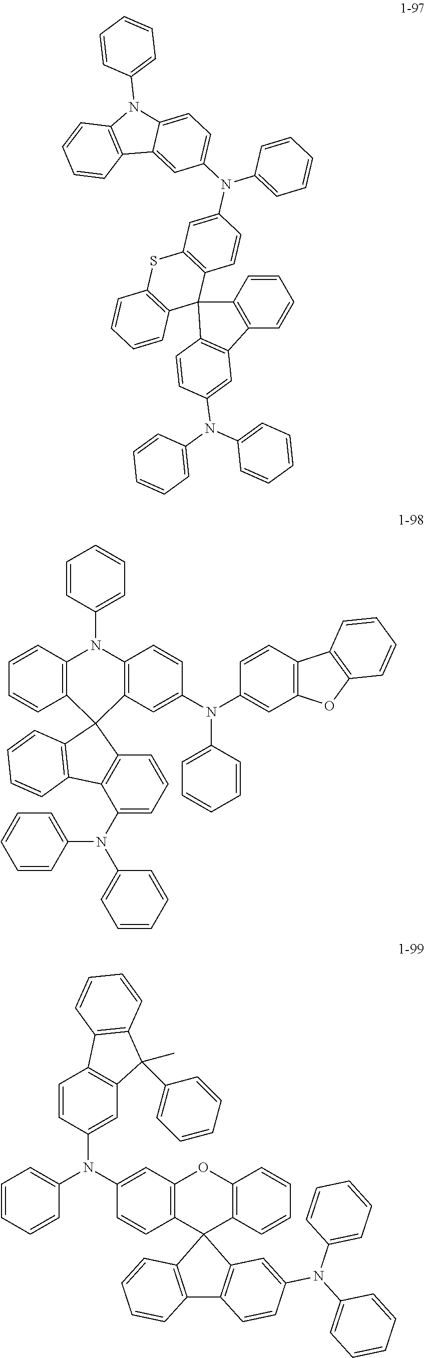

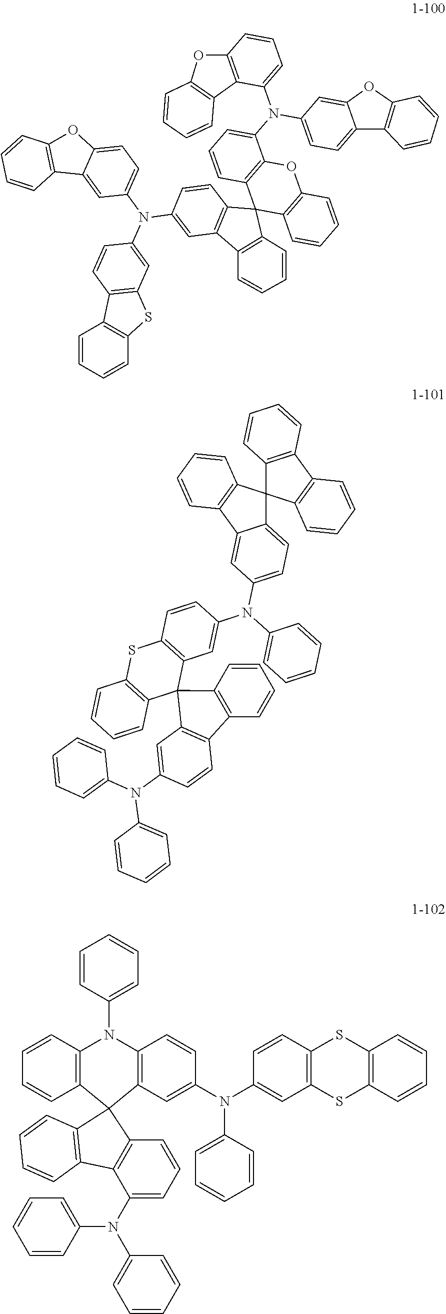 [SMILES: c1ccc2c(c1)Oc1c(N(c3ccc4c(c3)oc3ccccc34)c3cccc4oc5ccccc5c34)cccc1C21c2ccccc2-c2cc(N(c3ccc4c(c3)sc3ccccc34)c3ccc4oc5ccccc5c4c3)ccc21 ; c1ccc(N(c2ccc3c(c2)-c2ccccc2C32c3ccccc3-c3ccccc32)c2ccc3c(c2)C2(c4ccccc4S3)c3ccccc3-c3ccc(N(c4ccccc4)c4ccccc4)cc32)cc1 ; c1ccc(N(c2ccc3c(c2)Sc2ccccc2S3)c2ccc3c(c2)C2(c4ccccc4-c4c(N(c5ccccc5)c5ccccc5)cccc42)c2ccccc2N3c2ccccc2)cc1]

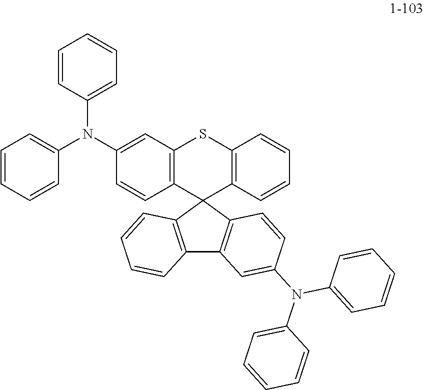 [SMILES: c1ccc(N(c2ccccc2)c2ccc3c(c2)Sc2ccccc2C32c3ccccc3-c3cc(N(c4ccccc4)c4ccccc4)ccc32)cc1]

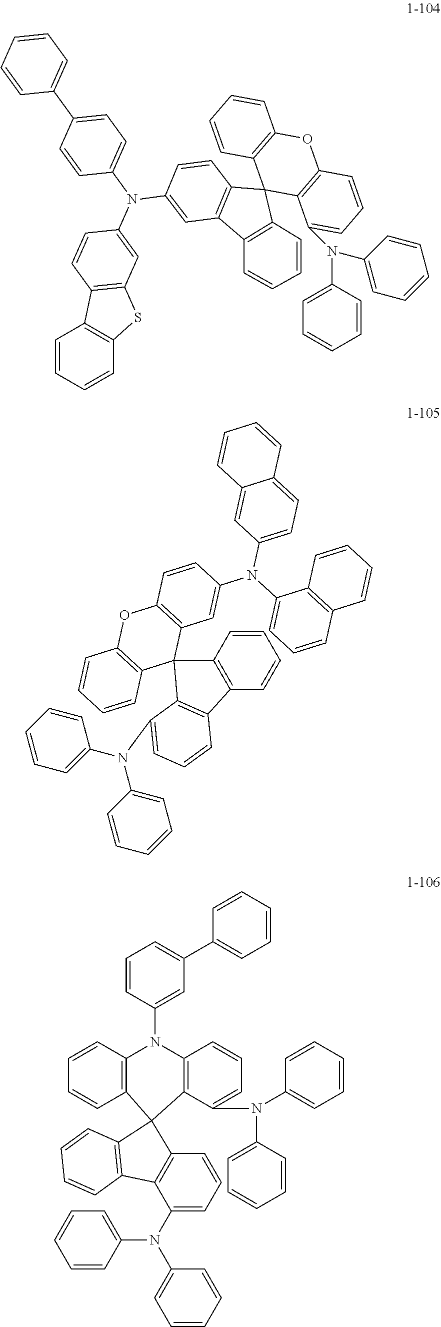 [SMILES: c1ccc(-c2ccc(N(c3ccc4c(c3)-c3ccccc3C43c4ccccc4Oc4cccc(N(c5ccccc5)c5ccccc5)c43)c3ccc4c(c3)sc3ccccc34)cc2)cc1 ; c1ccc(N(c2ccccc2)c2cccc3c2C2(c4ccccc4Oc4ccc(N(c5ccc6ccccc6c5)c5cccc6ccccc56)cc42)c2ccccc2-3)cc1 ; c1ccc(-c2cccc(N3c4ccccc4C4(c5ccccc5-c5c(N(c6ccccc6)c6ccccc6)cccc54)c4c(N(c5ccccc5)c5ccccc5)cccc43)c2)cc1]

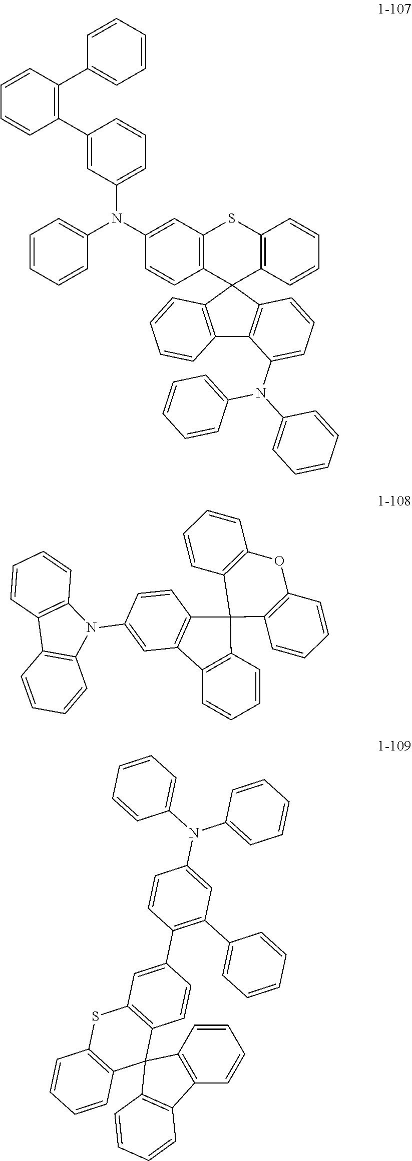 [SMILES: c1ccc(-c2ccccc2-c2cccc(N(c3ccccc3)c3ccc4c(c3)Sc3ccccc3C43c4ccccc4-c4c(N(c5ccccc5)c5ccccc5)cccc43)c2)cc1 ; c1ccc2c(c1)Oc1ccccc1C21c2ccccc2-c2cc(-n3c4ccccc4c4ccccc43)ccc21 ; c1ccc(-c2cc(N(c3ccccc3)c3ccccc3)ccc2-c2ccc3c(c2)Sc2ccccc2C32c3ccccc3-c3ccccc32)cc1]

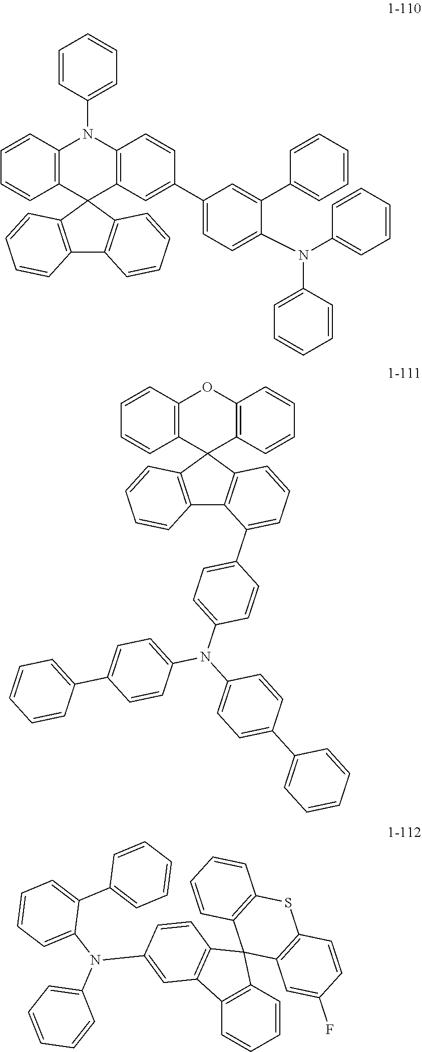 [SMILES: c1ccc(-c2cc(-c3ccc4c(c3)C3(c5ccccc5-c5ccccc53)c3ccccc3N4c3ccccc3)ccc2N(c2ccccc2)c2ccccc2)cc1 ; c1ccc(-c2ccc(N(c3ccc(-c4ccccc4)cc3)c3ccc(-c4cccc5c4-c4ccccc4C54c5ccccc5Oc5ccccc54)cc3)cc2)cc1 ; Fc1ccc2c(c1)C1(c3ccccc3S2)c2ccccc2-c2cc(N(c3ccccc3)c3ccccc3-c3ccccc3)ccc21]

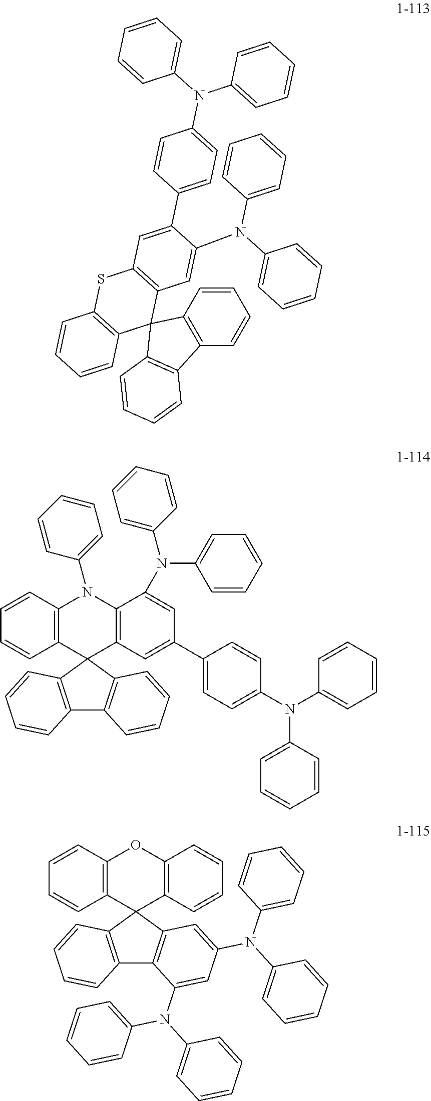

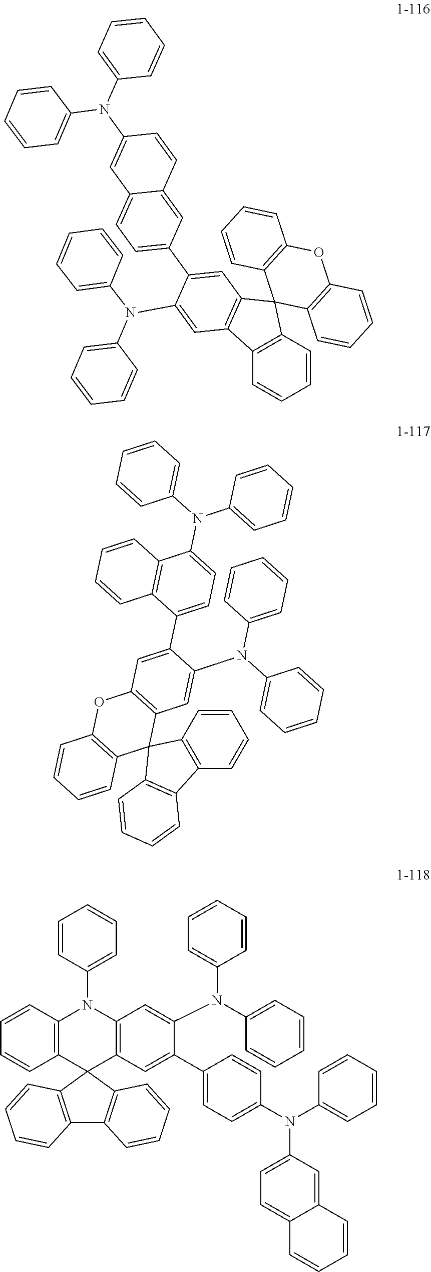 [SMILES: c1ccc(N(c2ccccc2)c2ccc3cc(-c4cc5c(cc4N(c4ccccc4)c4ccccc4)-c4ccccc4C54c5ccccc5Oc5ccccc54)ccc3c2)cc1 ; c1ccc(N(c2ccccc2)c2cc3c(cc2-c2ccc(N(c4ccccc4)c4ccccc4)c4ccccc24)Oc2ccccc2C32c3ccccc3-c3ccccc32)cc1 ; c1ccc(N(c2ccc(-c3cc4c(cc3N(c3ccccc3)c3ccccc3)N(c3ccccc3)c3ccccc3C43c4ccccc4-c4ccccc43)cc2)c2ccc3ccccc3c2)cc1]

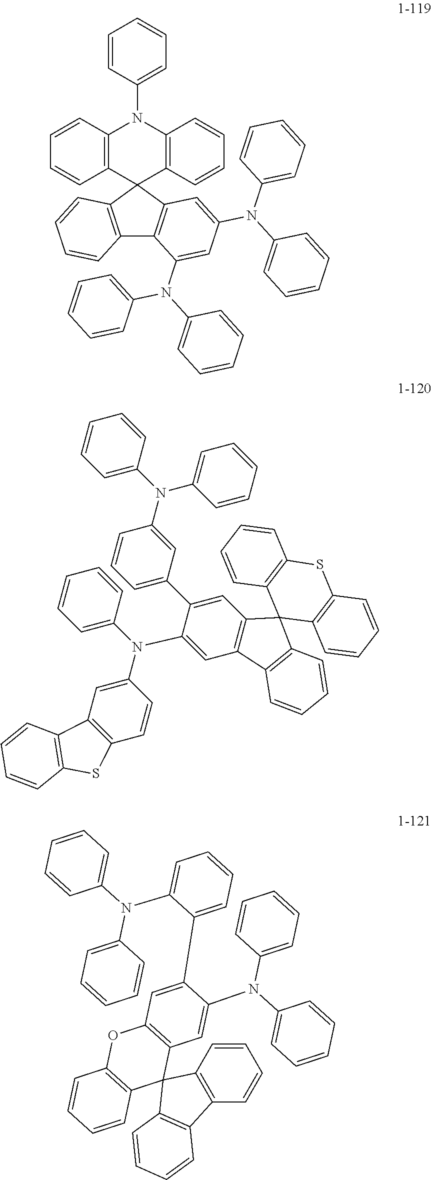 [SMILES: c1ccc(N(c2ccccc2)c2cc(N(c3ccccc3)c3ccccc3)c3c(c2)C2(c4ccccc4-3)c3ccccc3N(c3ccccc3)c3ccccc32)cc1 ; c1ccc(N(c2ccccc2)c2cccc(-c3cc4c(cc3N(c3ccccc3)c3ccc5sc6ccccc6c5c3)-c3ccccc3C43c4ccccc4Sc4ccccc43)c2)cc1 ; c1ccc(N(c2ccccc2)c2ccccc2-c2cc3c(cc2N(c2ccccc2)c2ccccc2)C2(c4ccccc4O3)c3ccccc3-c3ccccc32)cc1]

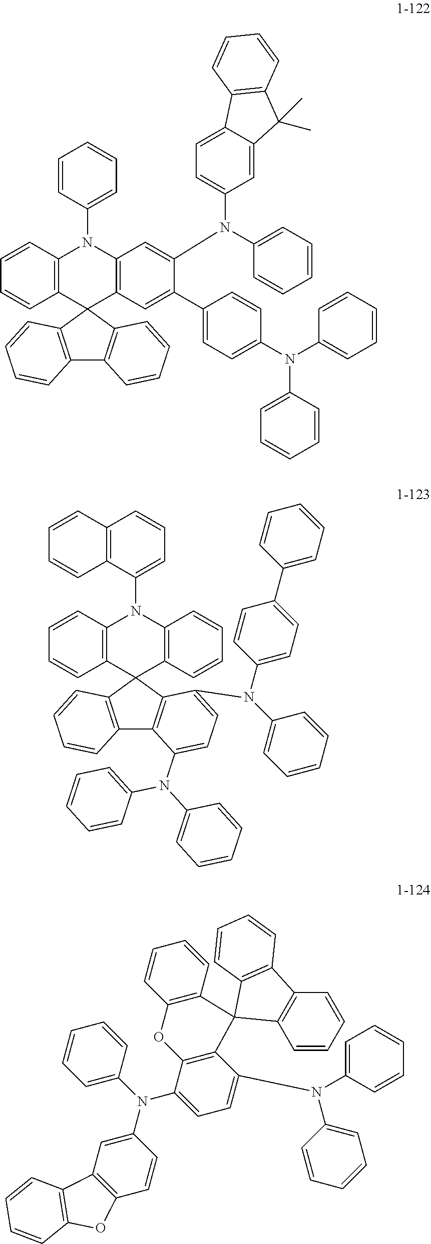

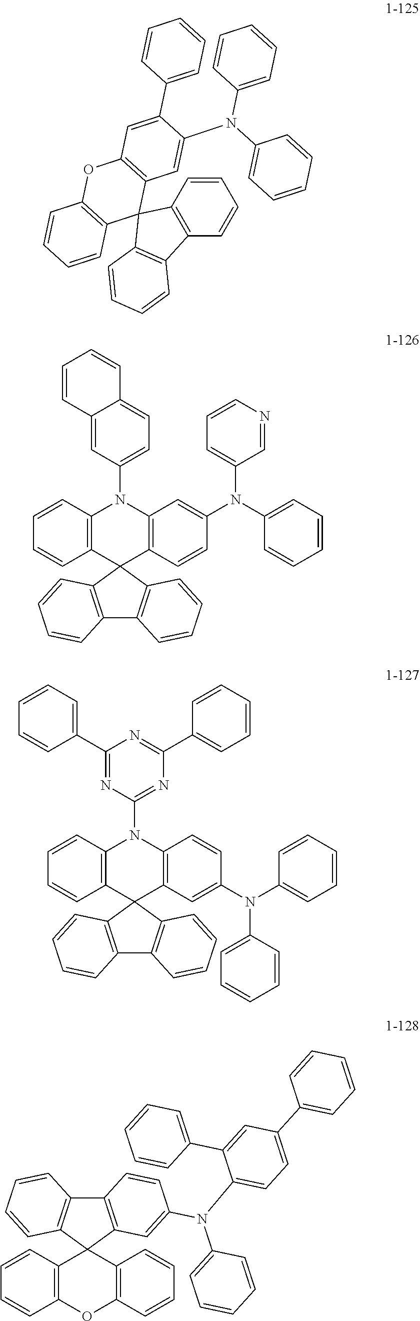 [SMILES: c1ccc(-c2cc3c(cc2N(c2ccccc2)c2ccccc2)C2(c4ccccc4O3)c3ccccc3-c3ccccc32)cc1 ; c1ccc(N(c2cccnc2)c2ccc3c(c2)N(c2ccc4ccccc4c2)c2ccccc2C32c3ccccc3-c3ccccc32)cc1 ; c1ccc(-c2nc(-c3ccccc3)nc(N3c4ccccc4C4(c5ccccc5-c5ccccc54)c4cc(N(c5ccccc5)c5ccccc5)ccc43)n2)cc1 ; c1ccc(-c2ccc(N(c3ccccc3)c3ccc4c(c3)C3(c5ccccc5Oc5ccccc53)c3ccccc3-4)c(-c3ccccc3)c2)cc1]

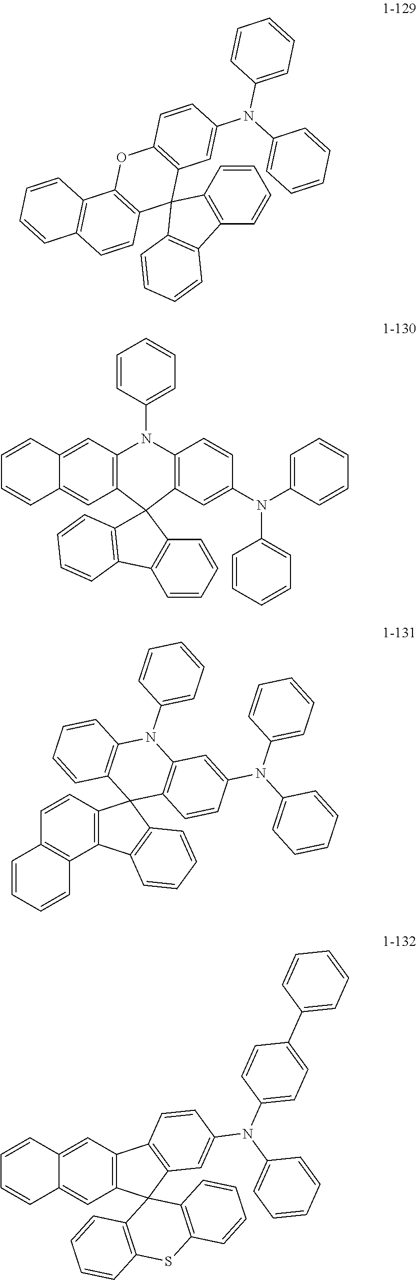 [SMILES: c1ccc(N(c2ccccc2)c2ccc3c(c2)C2(c4ccccc4-c4ccccc42)c2ccc4ccccc4c2O3)cc1 ; c1ccc(N(c2ccccc2)c2ccc3c(c2)C2(c4ccccc4-c4ccccc42)c2cc4ccccc4cc2N3c2ccccc2)cc1 ; c1ccc(N(c2ccccc2)c2ccc3c(c2)N(c2ccccc2)c2ccccc2C32c3ccccc3-c3c2ccc2ccccc32)cc1 ; c1ccc(-c2ccc(N(c3ccccc3)c3ccc4c(c3)C3(c5ccccc5Sc5ccccc53)c3cc5ccccc5cc3-4)cc2)cc1]

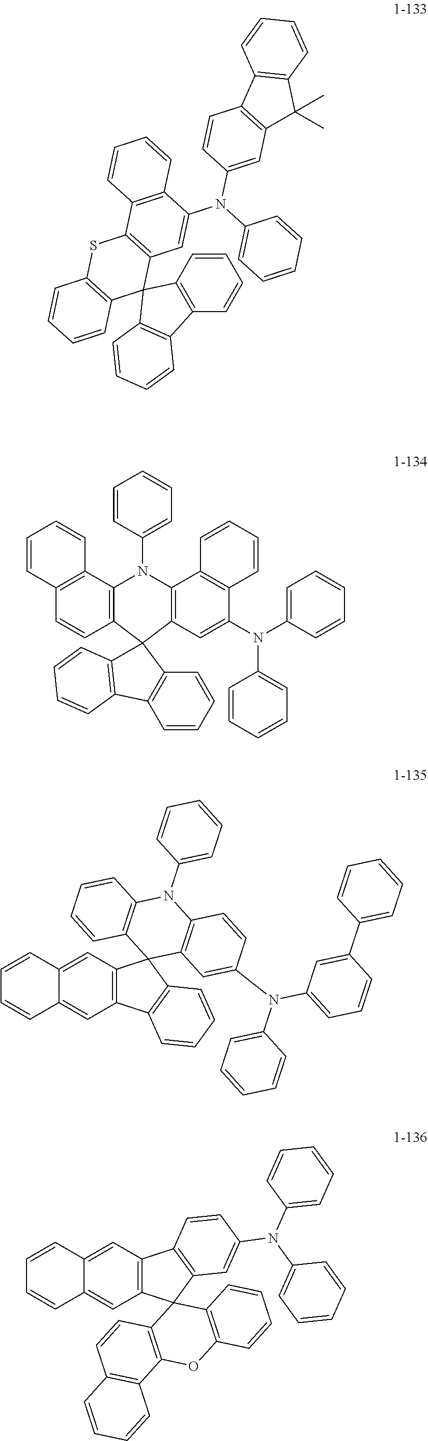 [SMILES: CC1(C)c2ccccc2-c2ccc(N(c3ccccc3)c3cc4c(c5ccccc35)Sc3ccccc3C43c4ccccc4-c4ccccc43)cc21 ; c1ccc(N(c2ccccc2)c2cc3c(c4ccccc24)N(c2ccccc2)c2c(ccc4ccccc24)C32c3ccccc3-c3ccccc32)cc1 ; c1ccc(-c2cccc(N(c3ccccc3)c3ccc4c(c3)C3(c5ccccc5-c5cc6ccccc6cc53)c3ccccc3N4c3ccccc3)c2)cc1 ; c1ccc(N(c2ccccc2)c2ccc3c(c2)C2(c4ccccc4Oc4c2ccc2ccccc42)c2cc4ccccc4cc2-3)cc1]

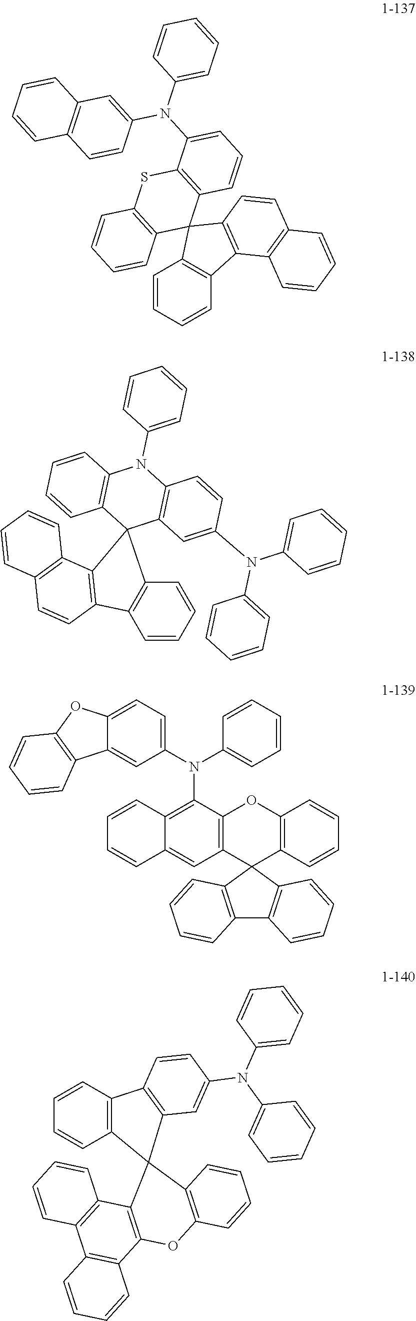

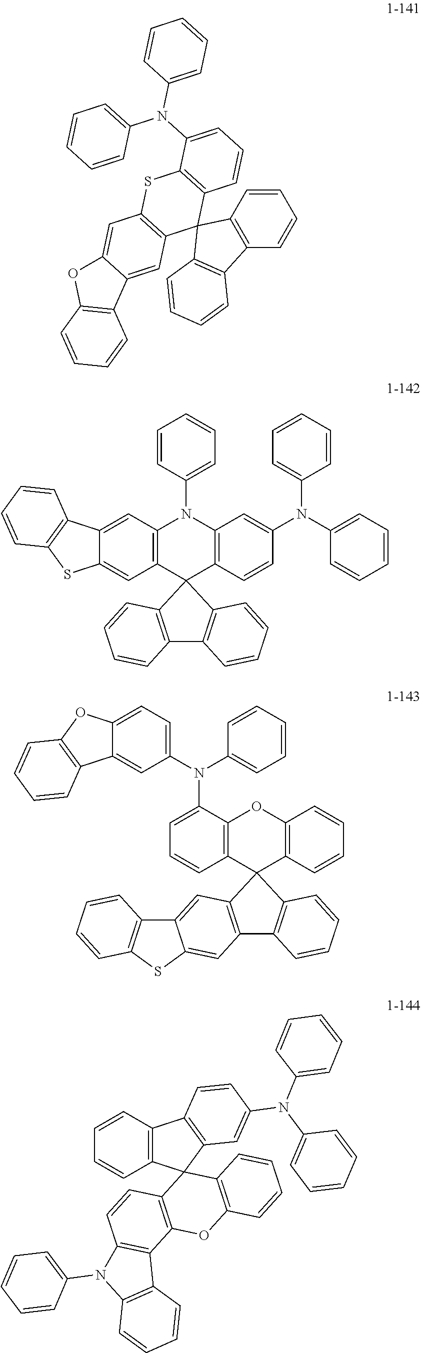

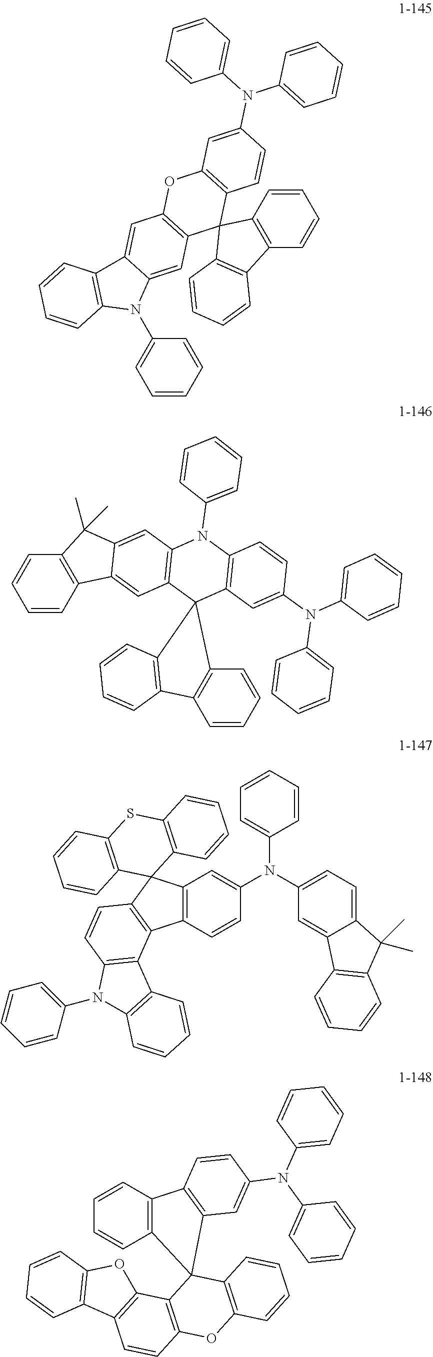 [SMILES: c1ccc(N(c2ccccc2)c2ccc3c(c2)Oc2cc4c5ccccc5n(-c5ccccc5)c4cc2C32c3ccccc3-c3ccccc32)cc1 ; CC1(C)c2ccccc2-c2cc3c(cc21)N(c1ccccc1)c1ccc(N(c2ccccc2)c2ccccc2)cc1C31c2ccccc2-c2ccccc21 ; CC1(C)c2ccccc2-c2cc(N(c3ccccc3)c3ccc4c(c3)C3(c5ccccc5Sc5ccccc53)c3ccc5c(c3-4)c3ccccc3n5-c3ccccc3)ccc21 ; c1ccc(N(c2ccccc2)c2ccc3c(c2)C2(c4ccccc4Oc4ccc5c(oc6ccccc65)c42)c2ccccc2-3)cc1]

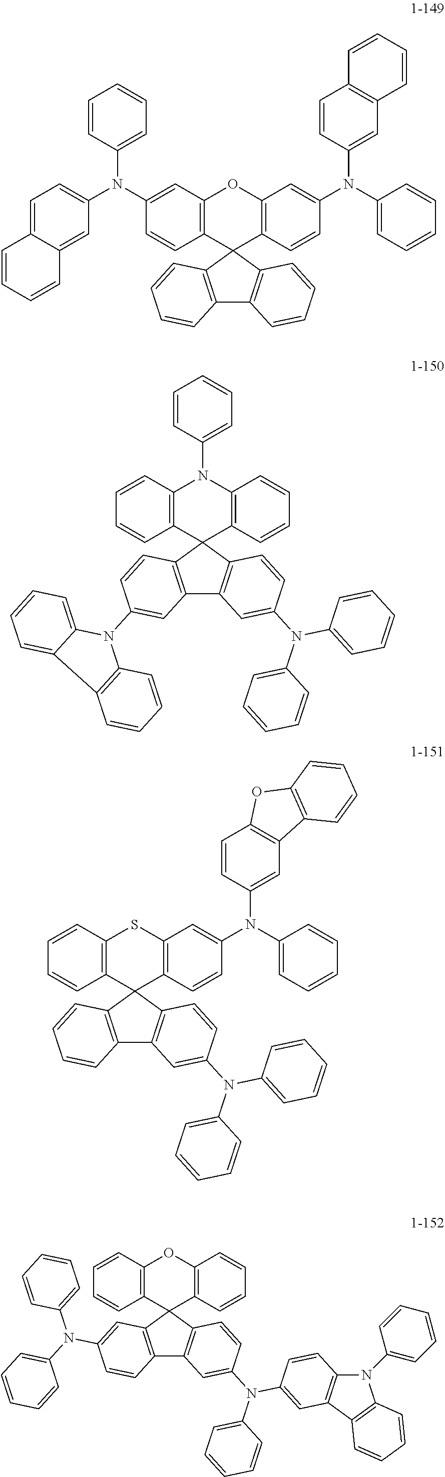

Also, the compound represented by Formula 2 is represented by any one of Formulas 2-1 to 2-3

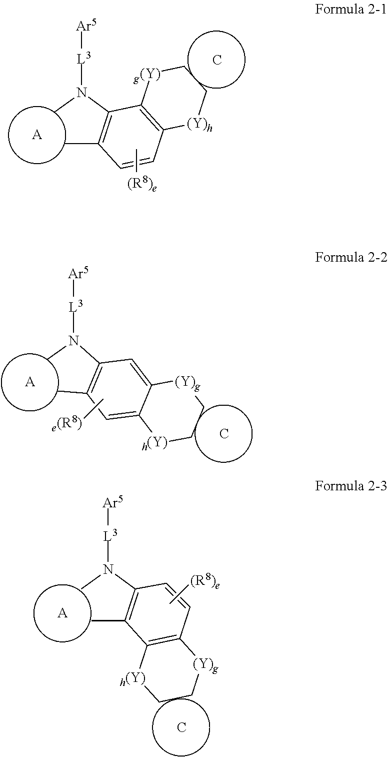

Wherein

1) Ring A, Ring C, R⁸, L³, Ar⁵ and Y are the same as defined above,

2) e is 0 to 2, g and h are 0 or 1, provided that g+h is 1.

Also, the host compound represented by Formula 2 is represented by any one of Formulas 2-4 to 2-27

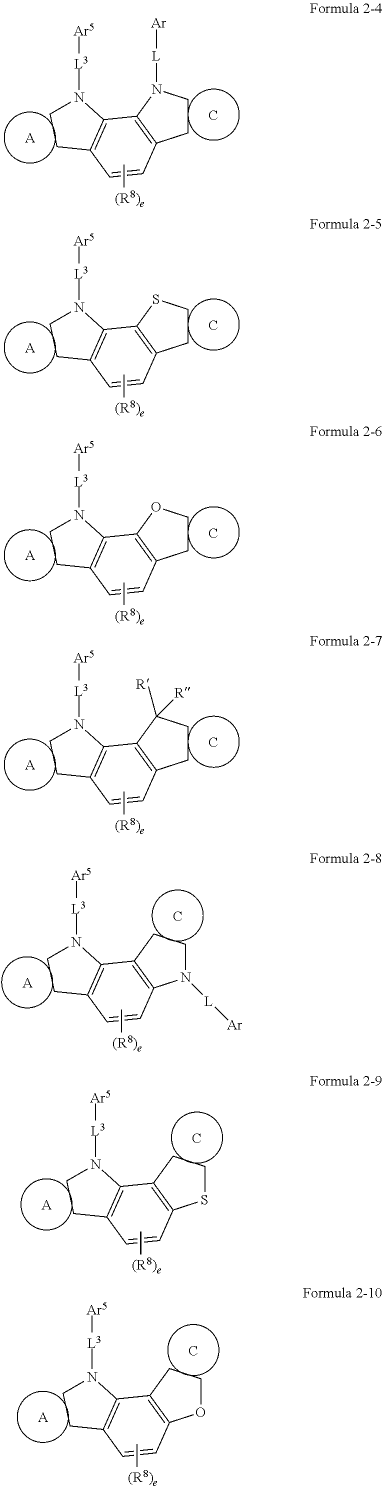

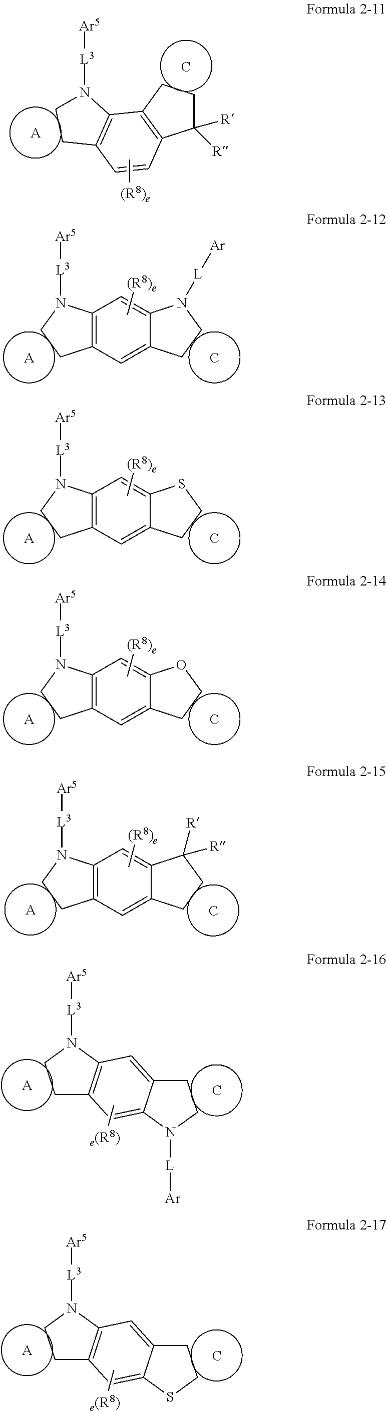

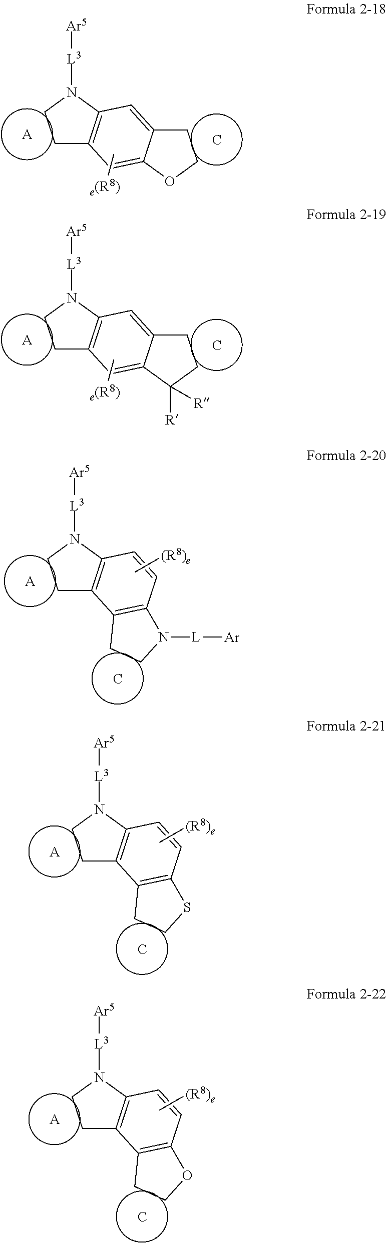

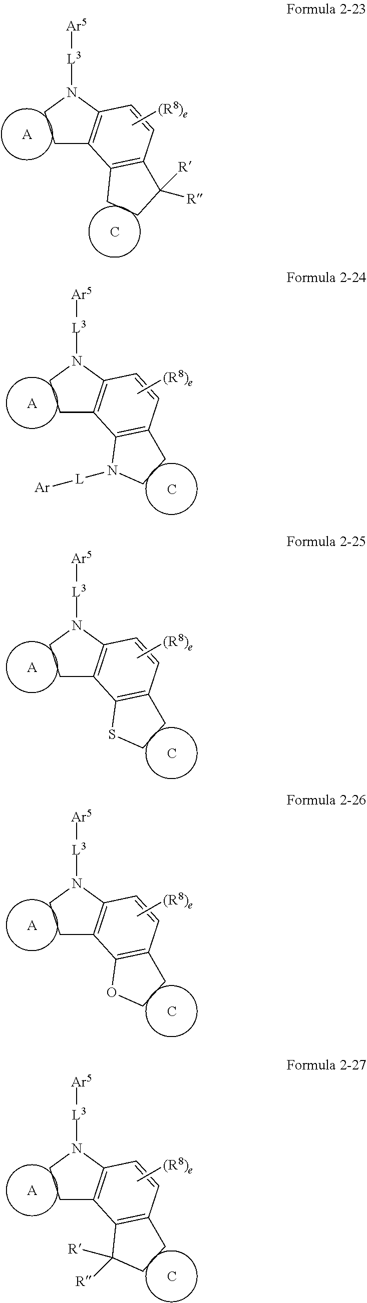

Wherein,

1) Ring A, Ring C, R⁸, L³ and Ar⁵ are the same as defined above,

2) e is 0 to 2,

3) R′ and R″ are the same as definition of R¹,

4) L is the same as the definition of L¹,

5) Ar is the same as the definition of Ar¹.

Also, at least one of R⁶ to R⁹ and Ar⁵ is represented by any one of Formulas A-1 to A-6

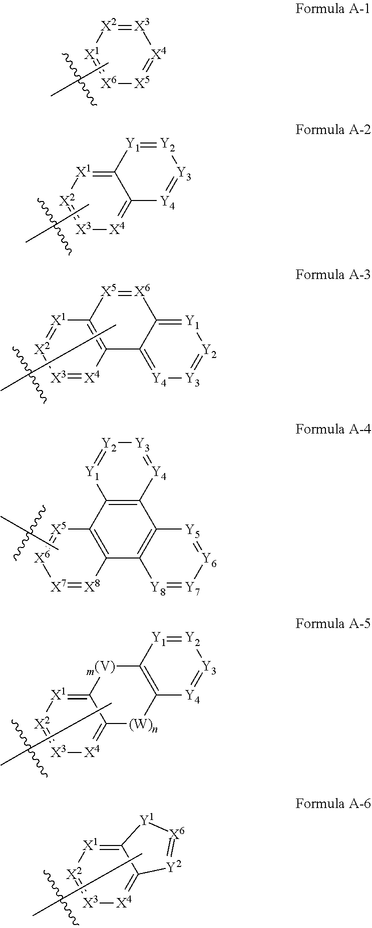

Wherein,

1) X¹, X², X³, X⁴, X⁵, X⁶, X⁷ and X⁸ are each independently C, C(R₁) or N,

2) Y₁, Y₂, Y₃, Y₄, Y₅, Y₆, Y₇ and Y₈ are each independently C(R₁) or N,

3) In Formula A-1, at least one of X¹ to X⁶ is N,

4) In Formula A-2, at least one of X¹ to X⁴ and Y₁ to Y₄ is N,

5) In Formula A-3, at least one of X¹ to X⁶ is N,

6) In Formula A-4, at least one of X⁵ to X⁸ and Y₁ to Y₈ is N,

7) In Formula A-5, at least one of X¹ to X⁴ is N,

8) In Formula A-6, X¹, X², X³, X⁴ and X⁶ are each independently C, C(R₁) or N, Y¹ is O, S, N-L′-Ar′ or CR¹³C¹⁴, Y² is N,

9) V and W are each independently O, S, N-L′-Ar′ or CR¹³C¹⁴,

10) m and n are each independently 0 or 1, provided that at least one of m and n is 1,

11) R¹, R¹³ and R¹⁴ are each independently selected from the group consisting of hydrogen; deuterium; halogen; a C₁-C₂₀ alkyl group; or a silane group unsubstituted or substituted with C₆-C₂₀ aryl group; cyano group; nitro group; C₁-C₂₀ alkylthio group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-C₂₀ aryl group; fluorenyl group; a C₂-C₂₀ heterocyclic group including at least one heteroatom of O, N, S, Si or P; a C₃-C₂₀ aliphatic ring; C₇-C₂₀ arylalkyl group; and C₈-C₂₀ aryl alkenyl group; adjacent R₁s, adjacent R¹³s and adjacent R¹⁴s may be bonded to each other to form a ring.

12) Wherein Ar′ is selected from the group consisting of a C₆-C₂₀ aryl group; fluorenyl group; C₂-C₂₀ heterocyclic group containing at least one heteroatom of O, N, S, Si or P; C₃-C₂₀ aliphatic ring; and combinations thereof,

13) wherein L′ is each independently selected from the group consisting of a single bond; C₆-C₂₀ arylene group; fluorenylene group; C₂-C₂₀ heterocyclic group containing at least one heteroatom of O, N, S, Si or P; and C₃-C₂₀ aliphatic ring group;

Specifically, the compound represented by Formula 2 may be any one of the following compounds.

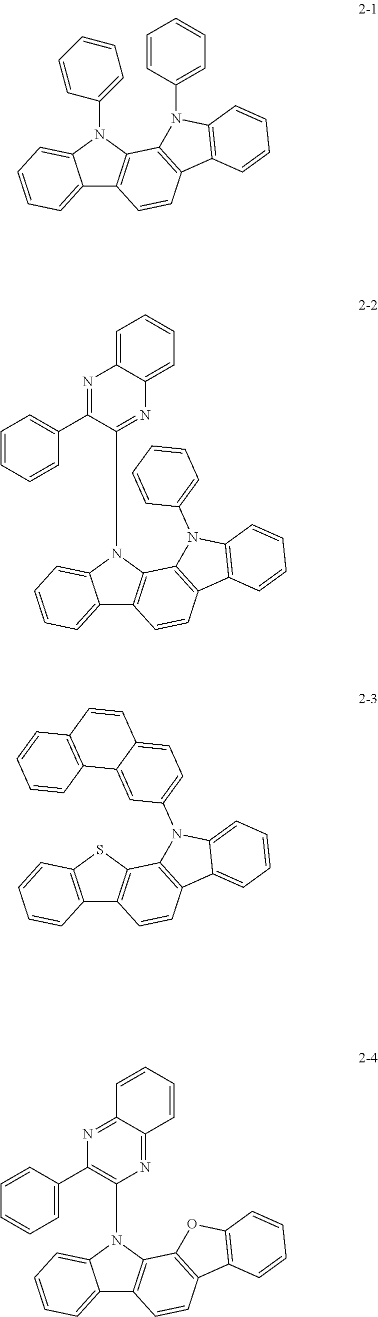 [SMILES: c1ccc(-n2c3ccccc3c3ccc4c5ccccc5n(-c5ccccc5)c4c32)cc1 ; c1ccc(-c2nc3ccccc3nc2-n2c3ccccc3c3ccc4c5ccccc5n(-c5ccccc5)c4c32)cc1 ; c1ccc2c(c1)ccc1ccc(-n3c4ccccc4c4ccc5c6ccccc6sc5c43)cc12 ; c1ccc(-c2nc3ccccc3nc2-n2c3ccccc3c3ccc4c5ccccc5oc4c32)cc1]

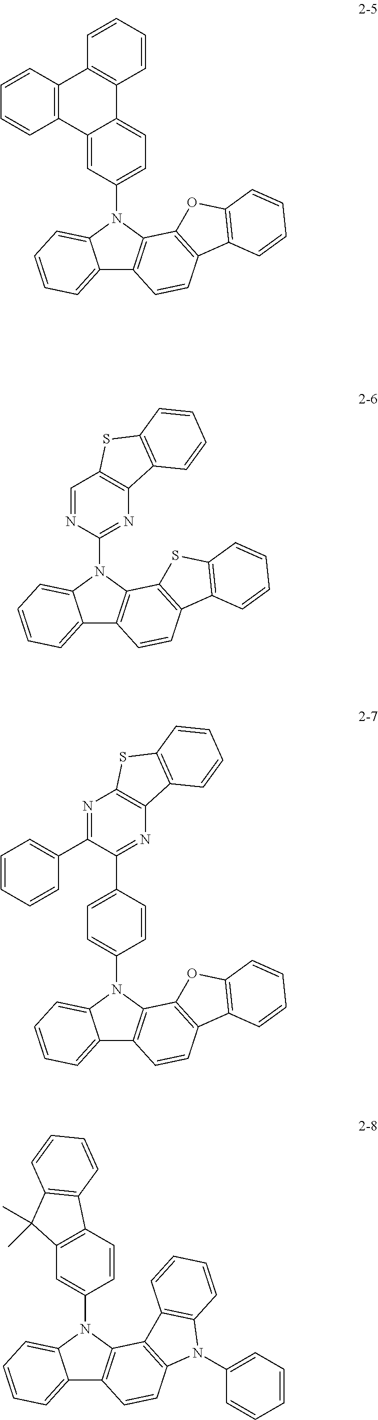 [SMILES: c1ccc2c(c1)oc1c2ccc2c3ccccc3n(-c3ccc4c5ccccc5c5ccccc5c4c3)c21 ; c1ccc2c(c1)sc1c2ccc2c3ccccc3n(-c3ncc4sc5ccccc5c4n3)c21 ; c1ccc(-c2nc3sc4ccccc4c3nc2-c2ccc(-n3c4ccccc4c4ccc5c6ccccc6oc5c43)cc2)cc1 ; CC1(C)c2ccccc2-c2ccc(-n3c4ccccc4c4ccc5c(c6ccccc6n5-c5ccccc5)c43)cc21]

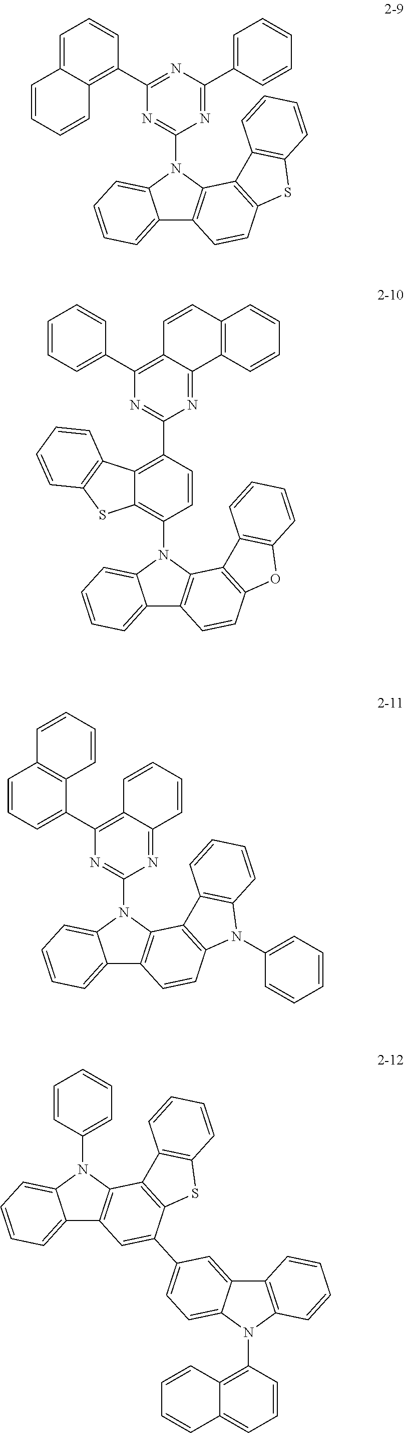 [SMILES: c1ccc(-c2nc(-c3cccc4ccccc34)nc(-n3c4ccccc4c4ccc5sc6ccccc6c5c43)n2)cc1 ; c1ccc(-c2nc(-c3ccc(-n4c5ccccc5c5ccc6oc7ccccc7c6c54)c4sc5ccccc5c34)nc3c2ccc2ccccc23)cc1 ; c1ccc(-n2c3ccccc3c3c2ccc2c4ccccc4n(-c4nc(-c5cccc6ccccc56)c5ccccc5n4)c23)cc1 ; c1ccc(-n2c3ccccc3c3cc(-c4ccc5c(c4)c4ccccc4n5-c4cccc5ccccc45)c4sc5ccccc5c4c32)cc1]

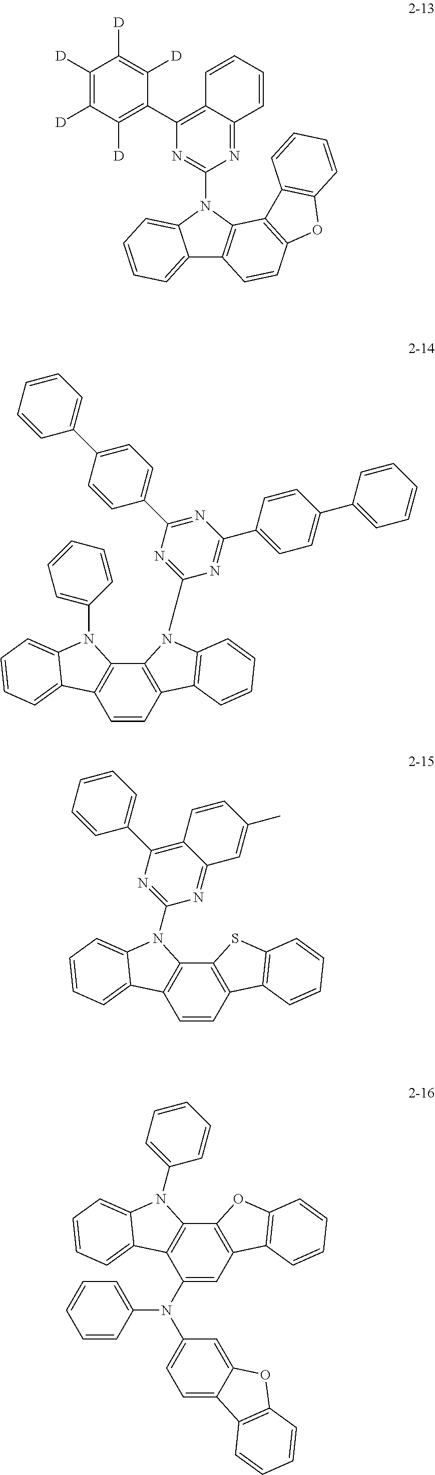 [SMILES: [2H]c1c([2H])c([2H])c(-c2nc(-n3c4ccccc4c4ccc5oc6ccccc6c5c43)nc3ccccc23)c([2H])c1[2H] ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1 ; Cc1ccc2c(-c3ccccc3)nc(-n3c4ccccc4c4ccc5c6ccccc6sc5c43)nc2c1 ; c1ccc(N(c2ccc3c(c2)oc2ccccc23)c2cc3c4ccccc4oc3c3c2c2ccccc2n3-c2ccccc2)cc1]

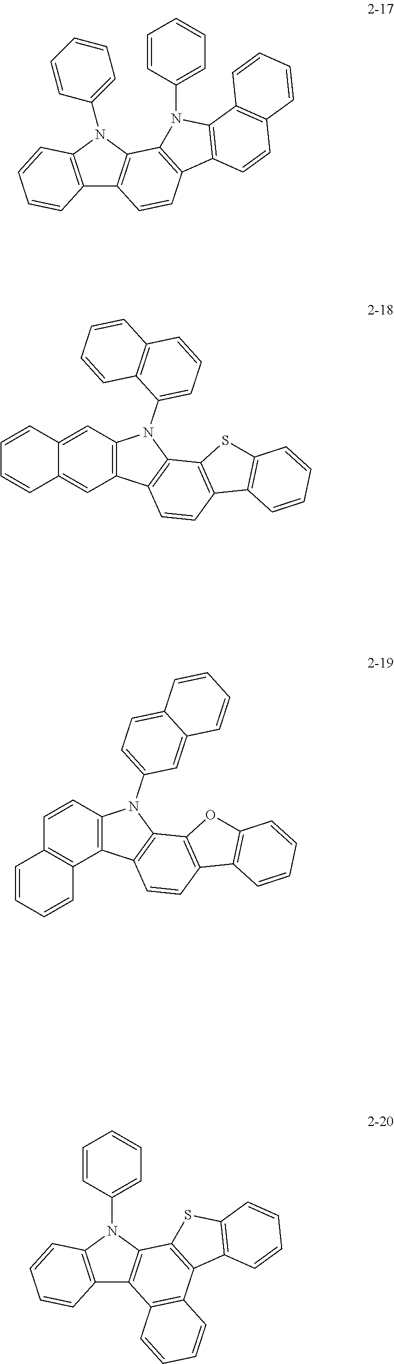 [SMILES: c1ccc(-n2c3ccccc3c3ccc4c5ccc6ccccc6c5n(-c5ccccc5)c4c32)cc1 ; c1ccc2cc3c(cc2c1)c1ccc2c4ccccc4sc2c1n3-c1cccc2ccccc12 ; c1ccc2cc(-n3c4ccc5ccccc5c4c4ccc5c6ccccc6oc5c43)ccc2c1 ; c1ccc(-n2c3ccccc3c3c4ccccc4c4c5ccccc5sc4c32)cc1]

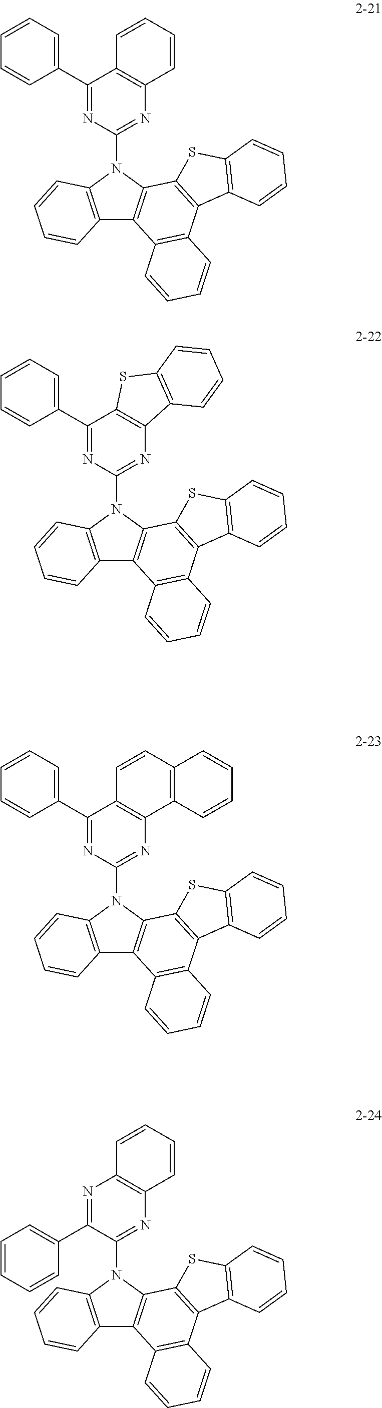

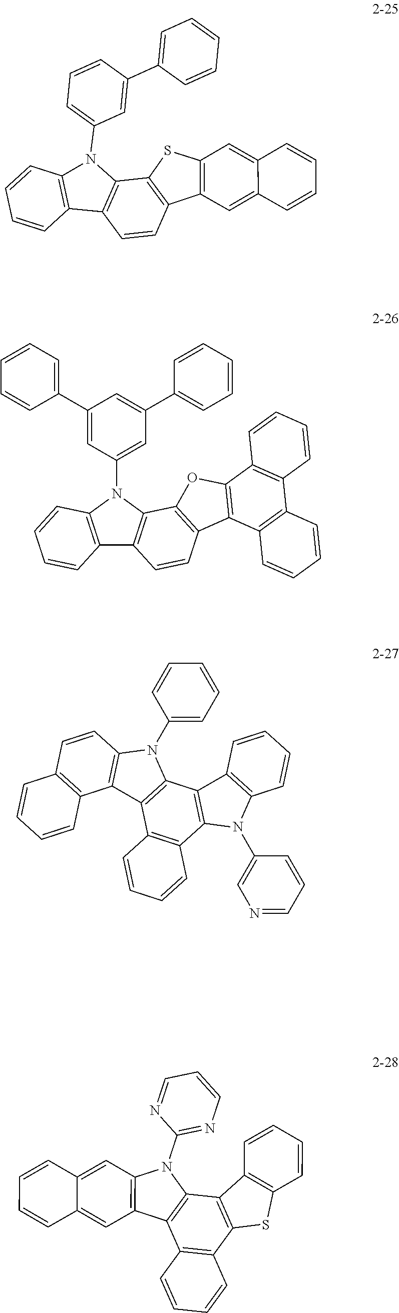 [SMILES: c1ccc(-c2cccc(-n3c4ccccc4c4ccc5c6cc7ccccc7cc6sc5c43)c2)cc1 ; c1ccc(-c2cc(-c3ccccc3)cc(-n3c4ccccc4c4ccc5c(oc6c7ccccc7c7ccccc7c56)c43)c2)cc1 ; c1ccc(-n2c3ccc4ccccc4c3c3c4ccccc4c4c(c5ccccc5n4-c4cccnc4)c32)cc1 ; c1cnc(-n2c3cc4ccccc4cc3c3c4ccccc4c4sc5ccccc5c4c32)nc1]

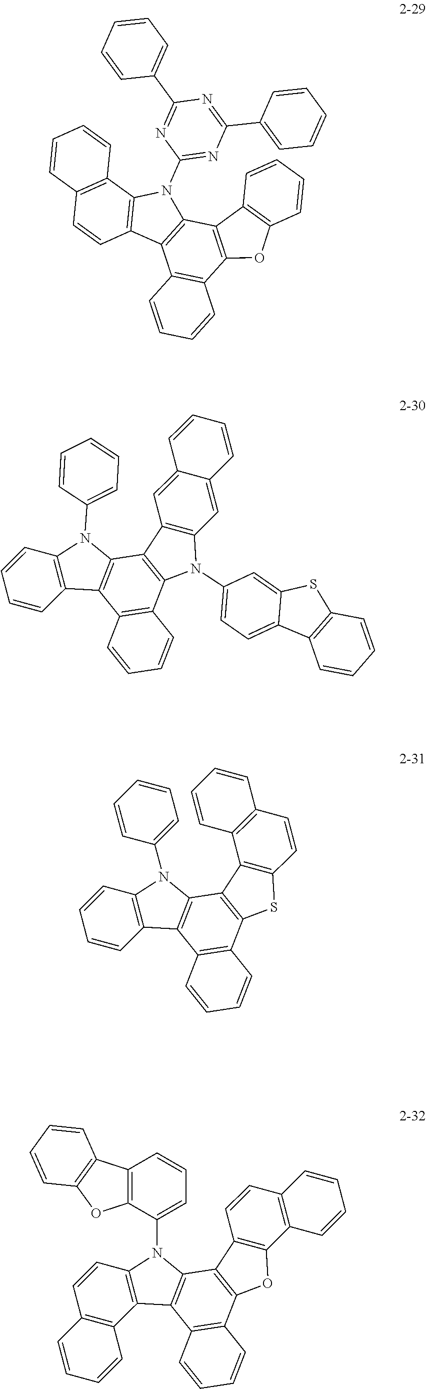 [SMILES: c1ccc(-c2nc(-c3ccccc3)nc(-n3c4c5ccccc5ccc4c4c5ccccc5c5oc6ccccc6c5c43)n2)cc1 ; c1ccc(-n2c3ccccc3c3c4ccccc4c4c(c5cc6ccccc6cc5n4-c4ccc5c(c4)sc4ccccc45)c32)cc1 ; c1ccc(-n2c3ccccc3c3c4ccccc4c4sc5ccc6ccccc6c5c4c32)cc1 ; c1ccc2c(c1)ccc1c2oc2c3ccccc3c3c4c5ccccc5ccc4n(-c4cccc5c4oc4ccccc45)c3c12]

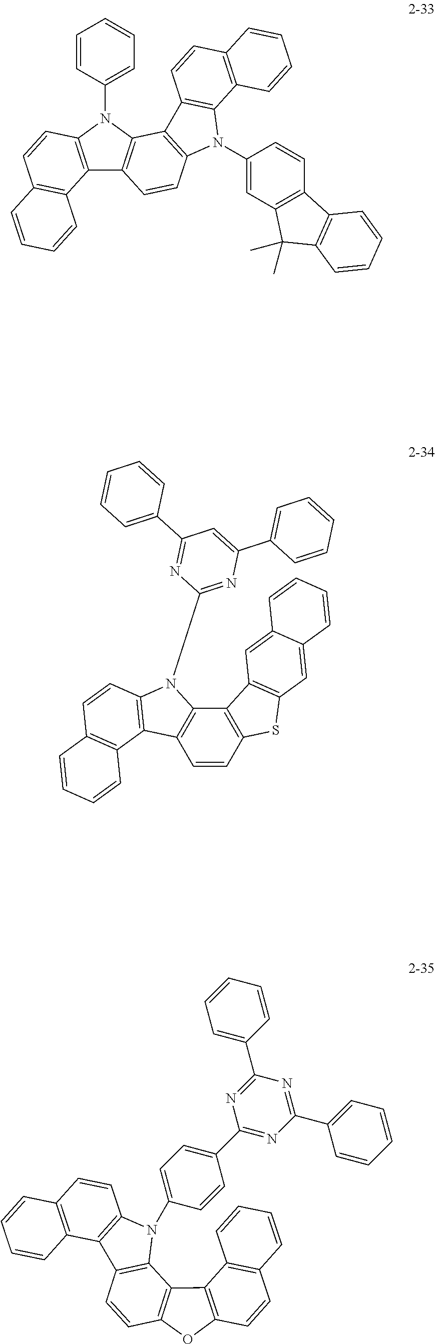 [SMILES: CC1(C)c2ccccc2-c2ccc(-n3c4ccc5c6c7ccccc7ccc6n(-c6ccccc6)c5c4c4ccc5ccccc5c43)cc21 ; c1ccc(-c2cc(-c3ccccc3)nc(-n3c4ccc5ccccc5c4c4ccc5sc6cc7ccccc7cc6c5c43)n2)cc1 ; c1ccc(-c2nc(-c3ccccc3)nc(-c3ccc(-n4c5ccc6ccccc6c5c5ccc6oc7ccc8ccccc8c7c6c54)cc3)n2)cc1]

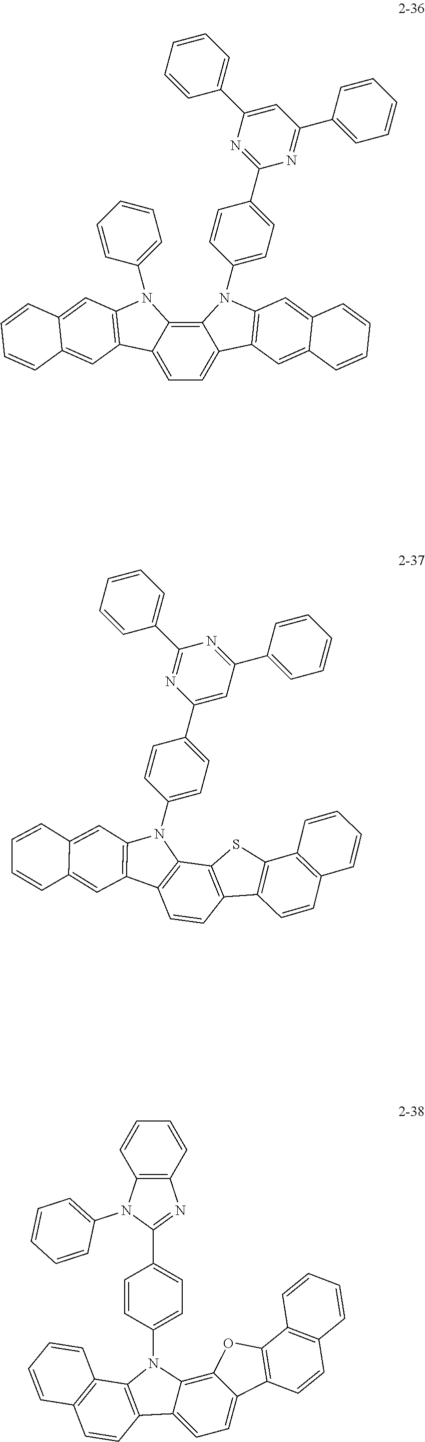 [SMILES: c1ccc(-c2cc(-c3ccccc3)nc(-c3ccc(-n4c5cc6ccccc6cc5c5ccc6c7cc8ccccc8cc7n(-c7ccccc7)c6c54)cc3)n2)cc1 ; c1ccc(-c2cc(-c3ccc(-n4c5cc6ccccc6cc5c5ccc6c7ccc8ccccc8c7sc6c54)cc3)nc(-c3ccccc3)n2)cc1 ; c1ccc(-n2c(-c3ccc(-n4c5c6ccccc6ccc5c5ccc6c7ccc8ccccc8c7oc6c54)cc3)nc3ccccc32)cc1]

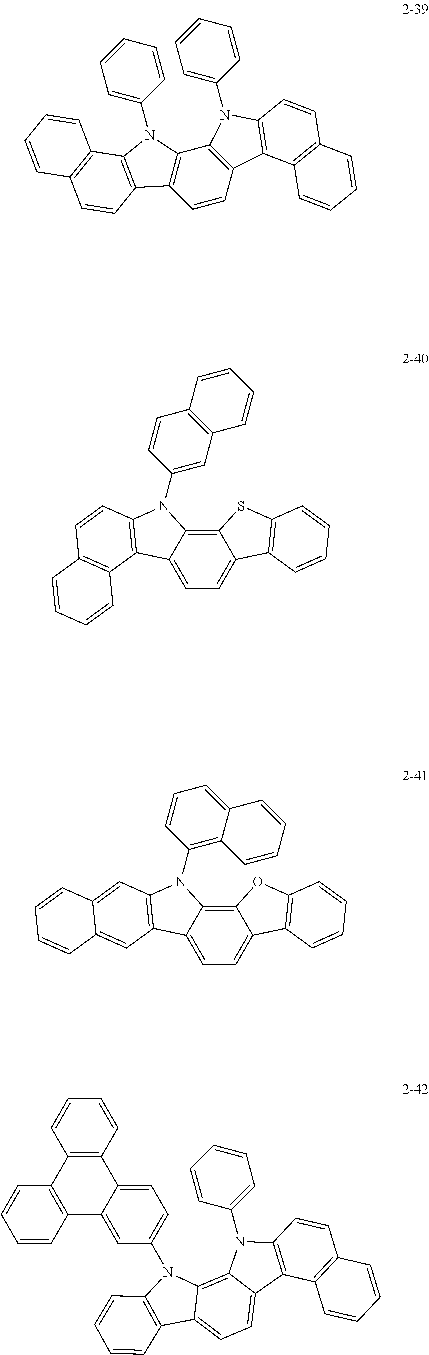 [SMILES: c1ccc(-n2c3ccc4ccccc4c3c3ccc4c5ccc6ccccc6c5n(-c5ccccc5)c4c32)cc1 ; c1ccc2cc(-n3c4ccc5ccccc5c4c4ccc5c6ccccc6sc5c43)ccc2c1 ; c1ccc2cc3c(cc2c1)c1ccc2c4ccccc4oc2c1n3-c1cccc2ccccc12 ; c1ccc(-n2c3ccc4ccccc4c3c3ccc4c5ccccc5n(-c5ccc6c7ccccc7c7ccccc7c6c5)c4c32)cc1]

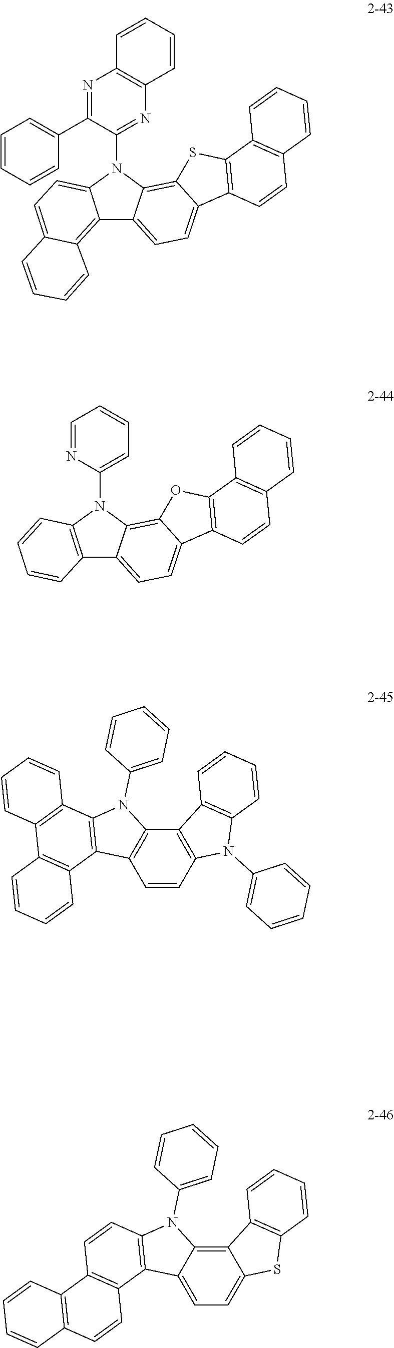 [SMILES: c1ccc(-c2nc3ccccc3nc2-n2c3ccc4ccccc4c3c3ccc4c5ccc6ccccc6c5sc4c32)cc1 ; c1ccc(-n2c3ccccc3c3ccc4c5ccc6ccccc6c5oc4c32)nc1 ; c1ccc(-n2c3ccccc3c3c2ccc2c4c5ccccc5c5ccccc5c4n(-c4ccccc4)c23)cc1 ; c1ccc(-n2c3ccc4c5ccccc5ccc4c3c3ccc4sc5ccccc5c4c32)cc1]

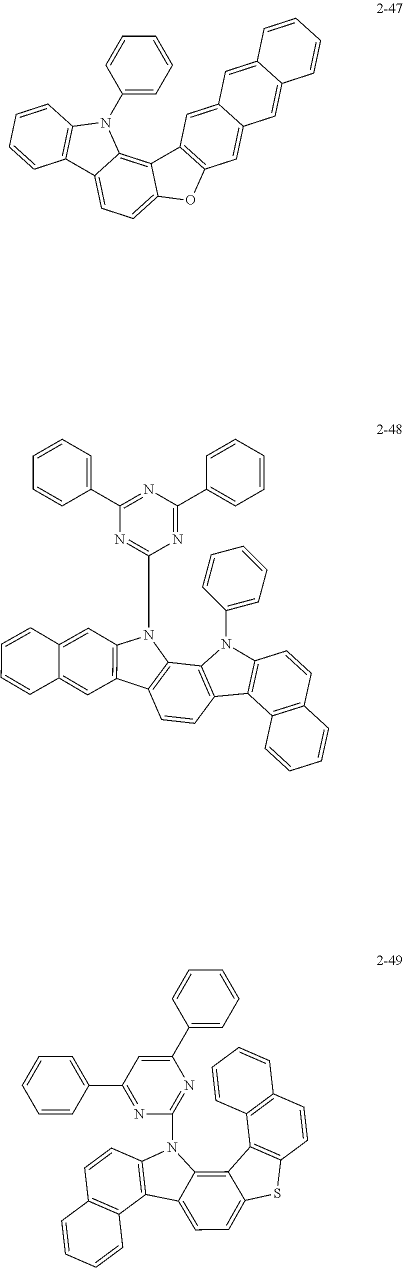 [SMILES: c1ccc(-n2c3ccccc3c3ccc4oc5cc6cc7ccccc7cc6cc5c4c32)cc1 ; c1ccc(-c2nc(-c3ccccc3)nc(-n3c4cc5ccccc5cc4c4ccc5c6c7ccccc7ccc6n(-c6ccccc6)c5c43)n2)cc1 ; c1ccc(-c2cc(-c3ccccc3)nc(-n3c4ccc5ccccc5c4c4ccc5sc6ccc7ccccc7c6c5c43)n2)cc1]

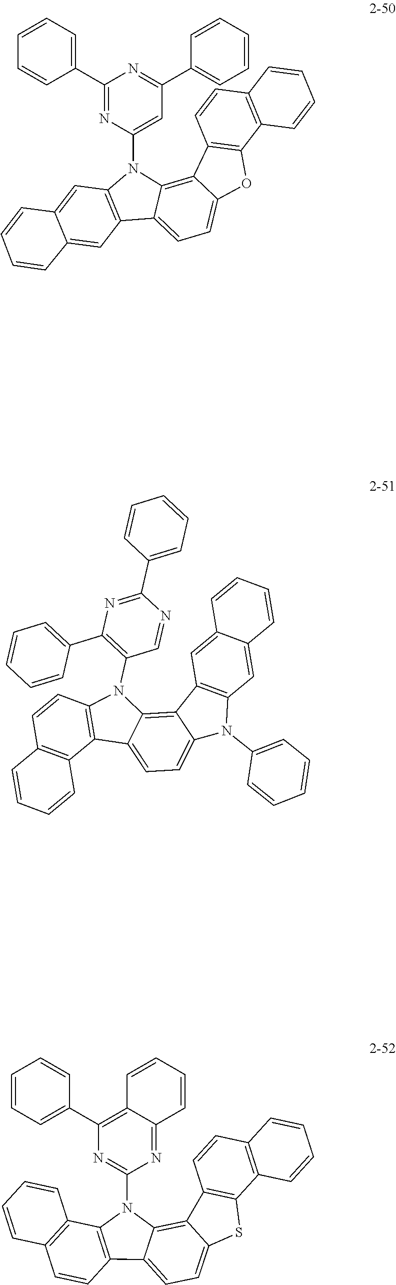 [SMILES: c1ccc(-c2cc(-n3c4cc5ccccc5cc4c4ccc5oc6c7ccccc7ccc6c5c43)nc(-c3ccccc3)n2)cc1 ; c1ccc(-c2ncc(-n3c4ccc5ccccc5c4c4ccc5c(c6cc7ccccc7cc6n5-c5ccccc5)c43)c(-c3ccccc3)n2)cc1 ; c1ccc(-c2nc(-n3c4c5ccccc5ccc4c4ccc5sc6c7ccccc7ccc6c5c43)nc3ccccc23)cc1]

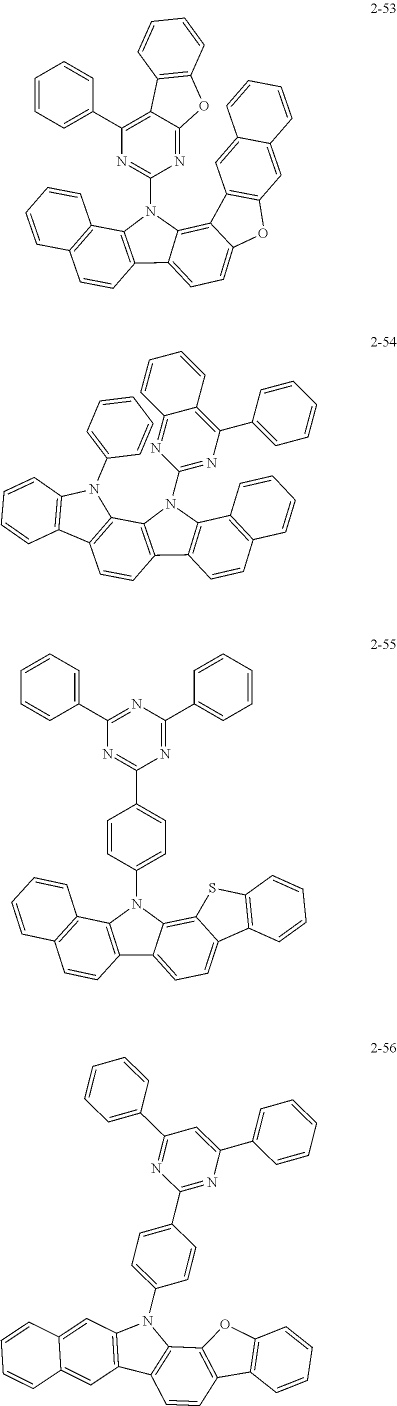 [SMILES: c1ccc(-c2nc(-n3c4c5ccccc5ccc4c4ccc5oc6cc7ccccc7cc6c5c43)nc3oc4ccccc4c23)cc1 ; c1ccc(-c2nc(-n3c4c5ccccc5ccc4c4ccc5c6ccccc6n(-c6ccccc6)c5c43)nc3ccccc23)cc1 ; c1ccc(-c2nc(-c3ccccc3)nc(-c3ccc(-n4c5c6ccccc6ccc5c5ccc6c7ccccc7sc6c54)cc3)n2)cc1 ; c1ccc(-c2cc(-c3ccccc3)nc(-c3ccc(-n4c5cc6ccccc6cc5c5ccc6c7ccccc7oc6c54)cc3)n2)cc1]

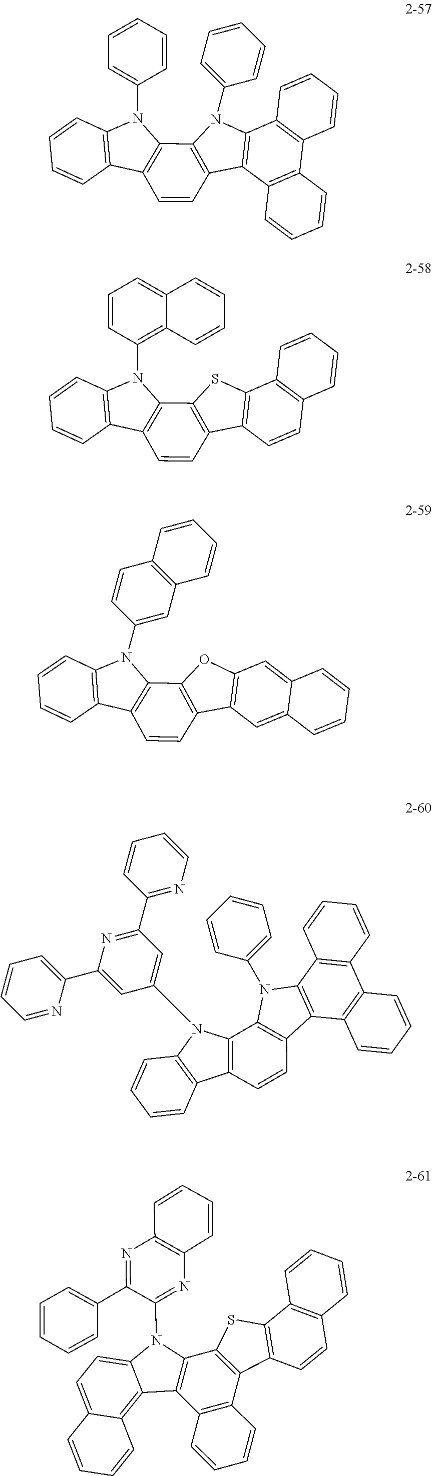 [SMILES: c1ccc(-n2c3ccccc3c3ccc4c5c6ccccc6c6ccccc6c5n(-c5ccccc5)c4c32)cc1 ; c1ccc2c(-n3c4ccccc4c4ccc5c6ccc7ccccc7c6sc5c43)cccc2c1 ; c1ccc2cc(-n3c4ccccc4c4ccc5c6cc7ccccc7cc6oc5c43)ccc2c1 ; c1ccc(-n2c3c4ccccc4c4ccccc4c3c3ccc4c5ccccc5n(-c5cc(-c6ccccn6)nc(-c6ccccn6)c5)c4c32)cc1 ; c1ccc(-c2nc3ccccc3nc2-n2c3ccc4ccccc4c3c3c4ccccc4c4c5ccc6ccccc6c5sc4c32)cc1]

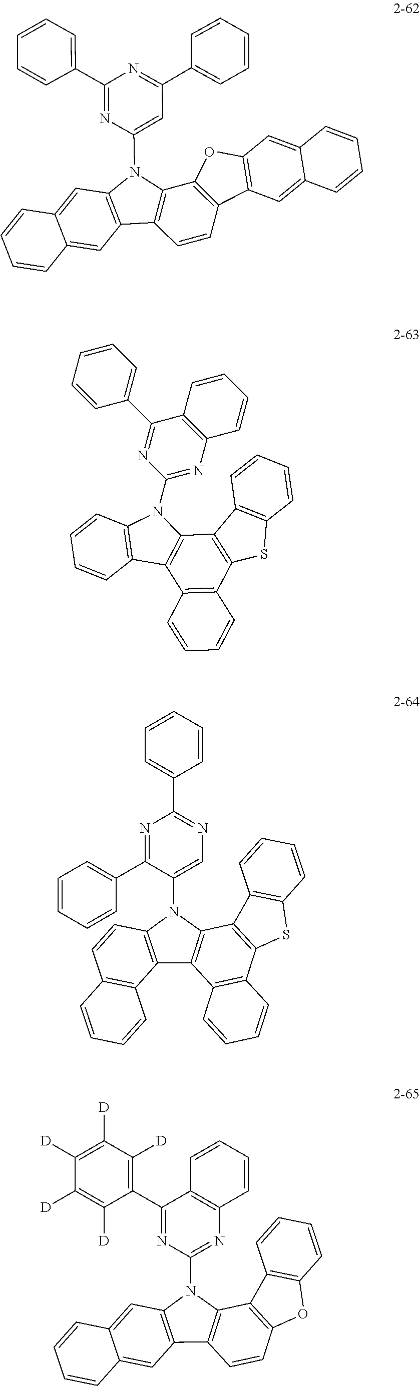 [SMILES: c1ccc(-c2cc(-n3c4cc5ccccc5cc4c4ccc5c6cc7ccccc7cc6oc5c43)nc(-c3ccccc3)n2)cc1 ; c1ccc(-c2nc(-n3c4ccccc4c4c5ccccc5c5sc6ccccc6c5c43)nc3ccccc23)cc1 ; c1ccc(-c2ncc(-n3c4ccc5ccccc5c4c4c5ccccc5c5sc6ccccc6c5c43)c(-c3ccccc3)n2)cc1 ; [2H]c1c([2H])c([2H])c(-c2nc(-n3c4cc5ccccc5cc4c4ccc5oc6ccccc6c5c43)nc3ccccc23)c([2H])c1[2H]]

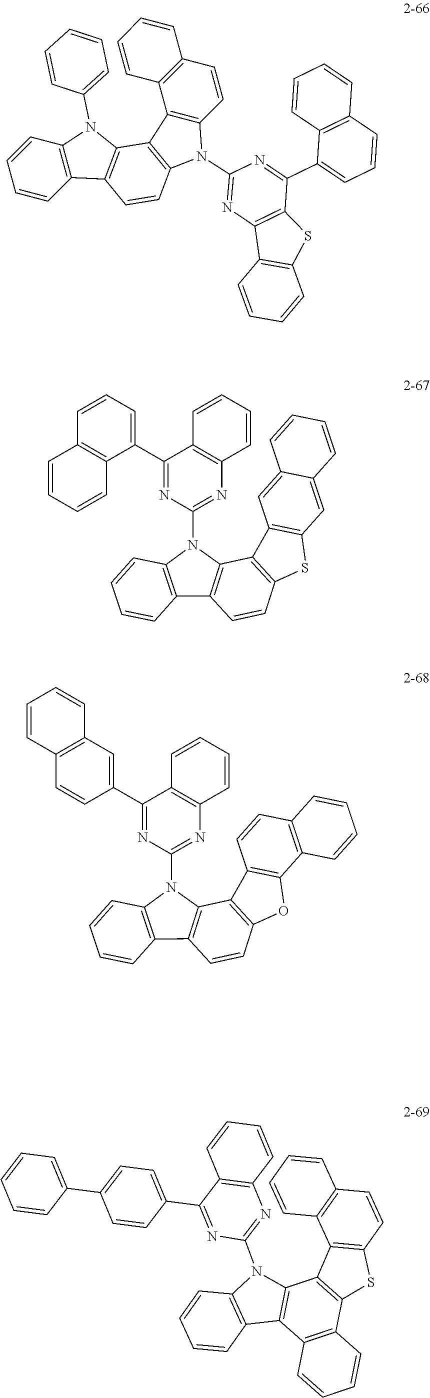 [SMILES: c1ccc(-n2c3ccccc3c3ccc4c(c5c6ccccc6ccc5n4-c4nc(-c5cccc6ccccc56)c5sc6ccccc6c5n4)c32)cc1 ; c1ccc2cc3c(cc2c1)sc1ccc2c4ccccc4n(-c4nc(-c5cccc6ccccc56)c5ccccc5n4)c2c13 ; c1ccc2cc(-c3nc(-n4c5ccccc5c5ccc6oc7c8ccccc8ccc7c6c54)nc4ccccc34)ccc2c1 ; c1ccc(-c2ccc(-c3nc(-n4c5ccccc5c5c6ccccc6c6sc7ccc8ccccc8c7c6c54)nc4ccccc34)cc2)cc1]

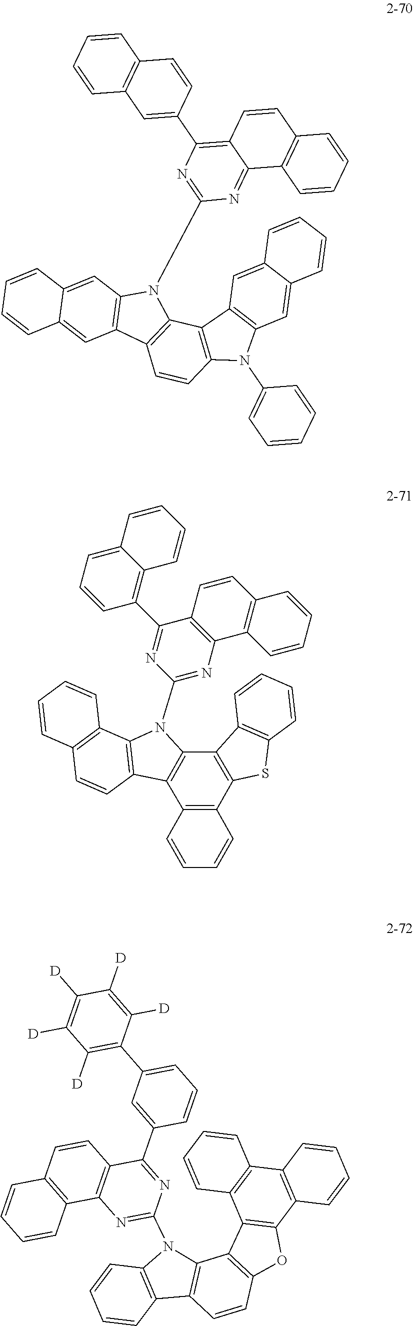 [SMILES: c1ccc(-n2c3cc4ccccc4cc3c3c2ccc2c4cc5ccccc5cc4n(-c4nc(-c5ccc6ccccc6c5)c5ccc6ccccc6c5n4)c23)cc1 ; c1ccc2c(-c3nc(-n4c5c6ccccc6ccc5c5c6ccccc6c6sc7ccccc7c6c54)nc4c3ccc3ccccc34)cccc2c1 ; [2H]c1c([2H])c([2H])c(-c2cccc(-c3nc(-n4c5ccccc5c5ccc6oc7c8ccccc8c8ccccc8c7c6c54)nc4c3ccc3ccccc34)c2)c([2H])c1[2H]]

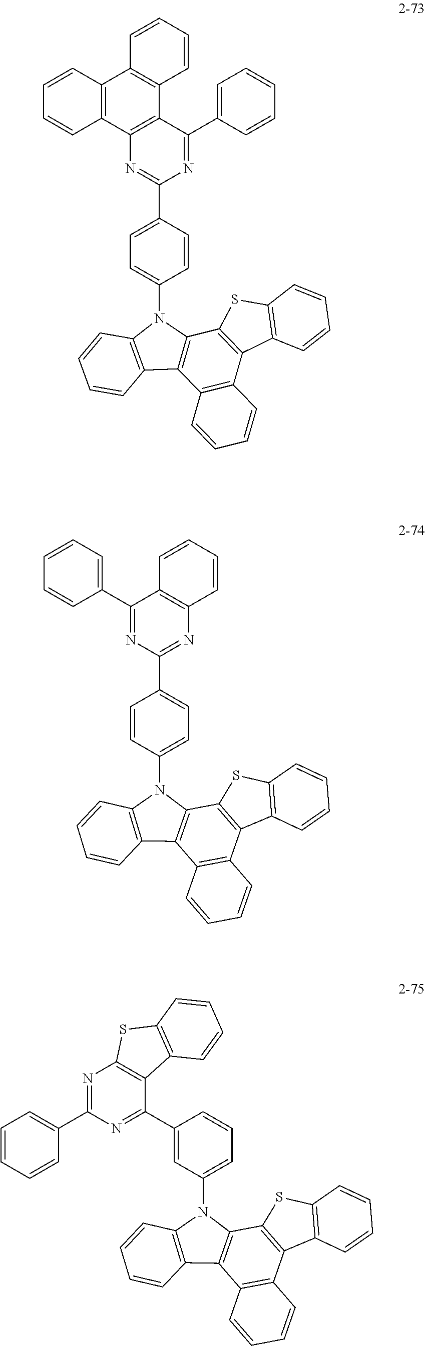 [SMILES: c1ccc(-c2nc(-c3ccc(-n4c5ccccc5c5c6ccccc6c6c7ccccc7sc6c54)cc3)nc3c4ccccc4c4ccccc4c23)cc1 ; c1ccc(-c2nc(-c3ccc(-n4c5ccccc5c5c6ccccc6c6c7ccccc7sc6c54)cc3)nc3ccccc23)cc1 ; c1ccc(-c2nc(-c3cccc(-n4c5ccccc5c5c6ccccc6c6c7ccccc7sc6c54)c3)c3c(n2)sc2ccccc23)cc1]

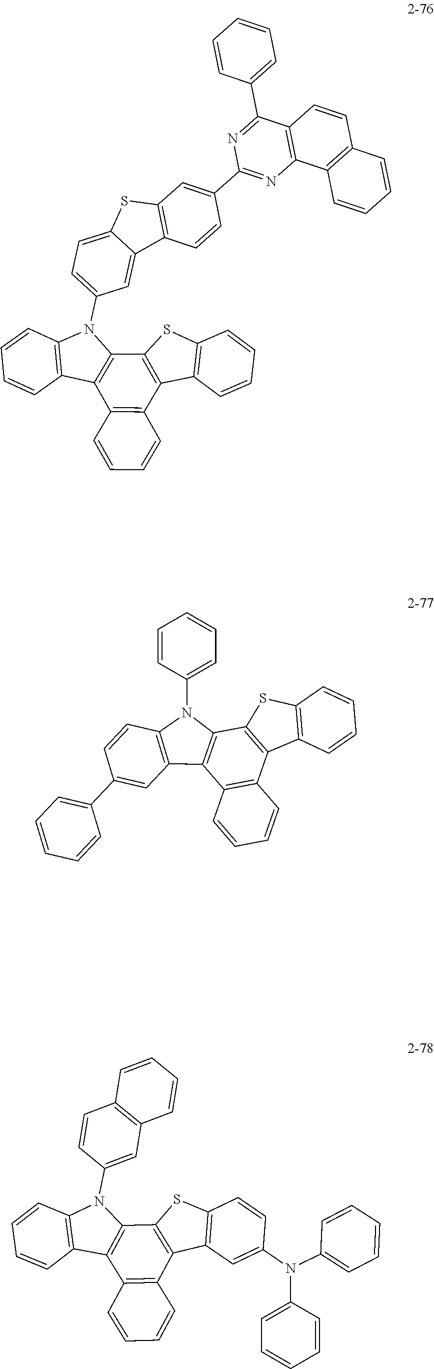 [SMILES: c1ccc(-c2nc(-c3ccc4c(c3)sc3ccc(-n5c6ccccc6c6c7ccccc7c7c8ccccc8sc7c65)cc34)nc3c2ccc2ccccc23)cc1 ; c1ccc(-c2ccc3c(c2)c2c4ccccc4c4c5ccccc5sc4c2n3-c2ccccc2)cc1 ; c1ccc(N(c2ccccc2)c2ccc3sc4c(c3c2)c2ccccc2c2c3ccccc3n(-c3ccc5ccccc5c3)c42)cc1]

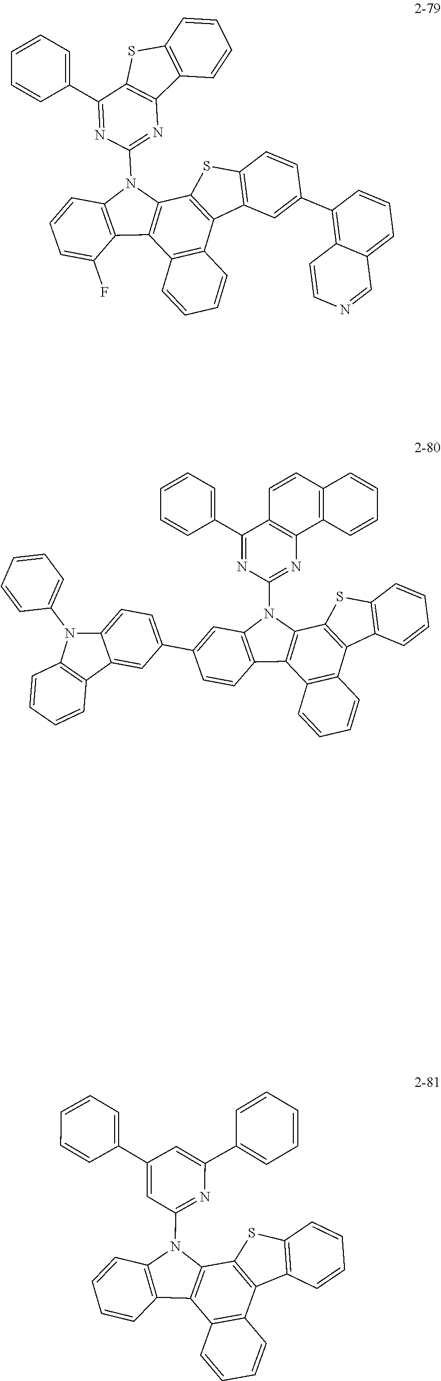 [SMILES: Fc1cccc2c1c1c3ccccc3c3c4cc(-c5cccc6cnccc56)ccc4sc3c1n2-c1nc(-c2ccccc2)c2sc3ccccc3c2n1 ; c1ccc(-c2nc(-n3c4cc(-c5ccc6c(c5)c5ccccc5n6-c5ccccc5)ccc4c4c5ccccc5c5c6ccccc6sc5c43)nc3c2ccc2ccccc23)cc1 ; c1ccc(-c2cc(-c3ccccc3)nc(-n3c4ccccc4c4c5ccccc5c5c6ccccc6sc5c43)c2)cc1]

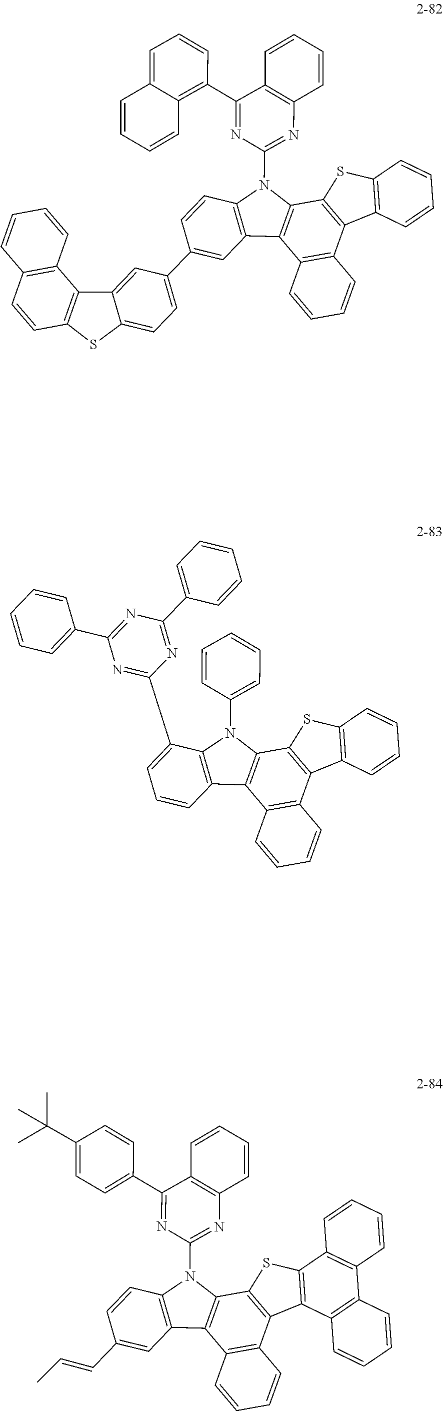 [SMILES: c1ccc2c(-c3nc(-n4c5ccc(-c6ccc7sc8ccc9ccccc9c8c7c6)cc5c5c6ccccc6c6c7ccccc7sc6c54)nc4ccccc34)cccc2c1 ; c1ccc(-c2nc(-c3ccccc3)nc(-c3cccc4c5c6ccccc6c6c7ccccc7sc6c5n(-c5ccccc5)c34)n2)cc1 ; C/C=C/c1ccc2c(c1)c1c3ccccc3c3c(sc4c5ccccc5c5ccccc5c43)c1n2-c1nc(-c2ccc(C(C)(C)C)cc2)c2ccccc2n1]

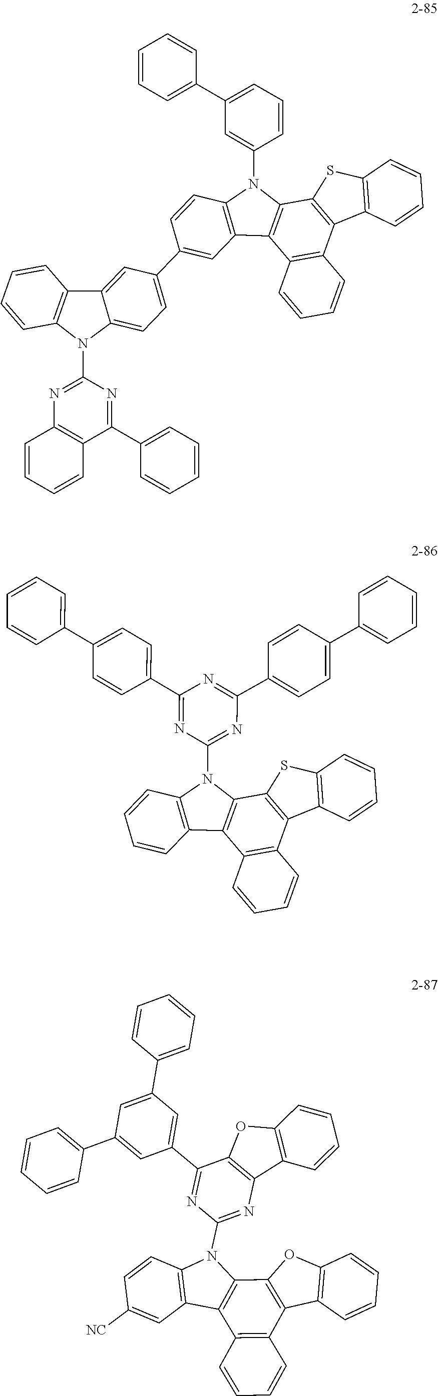 [SMILES: c1ccc(-c2cccc(-n3c4ccc(-c5ccc6c(c5)c5ccccc5n6-c5nc(-c6ccccc6)c6ccccc6n5)cc4c4c5ccccc5c5c6ccccc6sc5c43)c2)cc1 ; c1ccc(-c2ccc(-c3nc(-c4ccc(-c5ccccc5)cc4)nc(-n4c5ccccc5c5c6ccccc6c6c7ccccc7sc6c54)n3)cc2)cc1 ; N#Cc1ccc2c(c1)c1c3ccccc3c3c4ccccc4oc3c1n2-c1nc(-c2cc(-c3ccccc3)cc(-c3ccccc3)c2)c2oc3ccccc3c2n1]

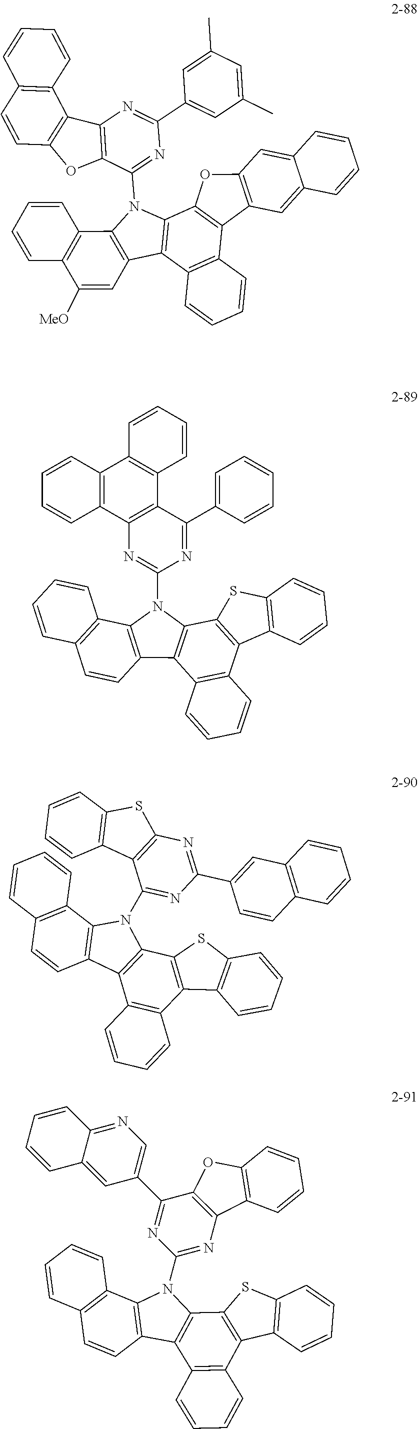 [SMILES: COc1cc2c3c4ccccc4c4c5cc6ccccc6cc5oc4c3n(-c3nc(-c4cc(C)cc(C)c4)nc4c3oc3ccc5ccccc5c34)c2c2ccccc12 ; c1ccc(-c2nc(-n3c4c5ccccc5ccc4c4c5ccccc5c5c6ccccc6sc5c43)nc3c4ccccc4c4ccccc4c23)cc1 ; c1ccc2cc(-c3nc(-n4c5c6ccccc6ccc5c5c6ccccc6c6c7ccccc7sc6c54)c4c(n3)sc3ccccc34)ccc2c1 ; c1ccc2ncc(-c3nc(-n4c5c6ccccc6ccc5c5c6ccccc6c6c7ccccc7sc6c54)nc4c3oc3ccccc34)cc2c1]

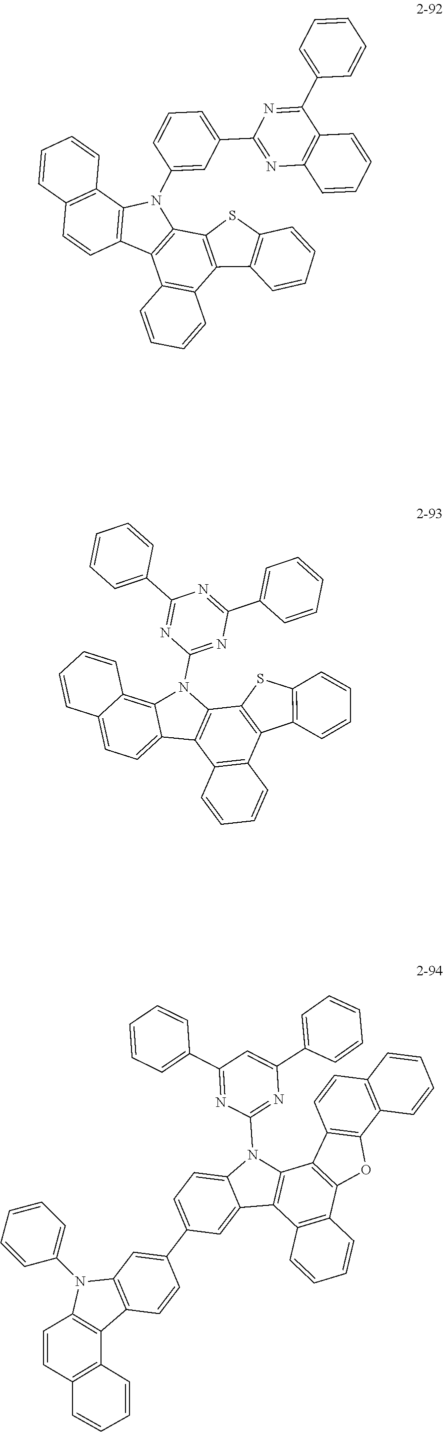 [SMILES: c1ccc(-c2nc(-c3cccc(-n4c5c6ccccc6ccc5c5c6ccccc6c6c7ccccc7sc6c54)c3)nc3ccccc23)cc1 ; c1ccc(-c2nc(-c3ccccc3)nc(-n3c4c5ccccc5ccc4c4c5ccccc5c5c6ccccc6sc5c43)n2)cc1 ; c1ccc(-c2cc(-c3ccccc3)nc(-n3c4ccc(-c5ccc6c7c8ccccc8ccc7n(-c7ccccc7)c6c5)cc4c4c5ccccc5c5oc6c7ccccc7ccc6c5c43)n2)cc1]

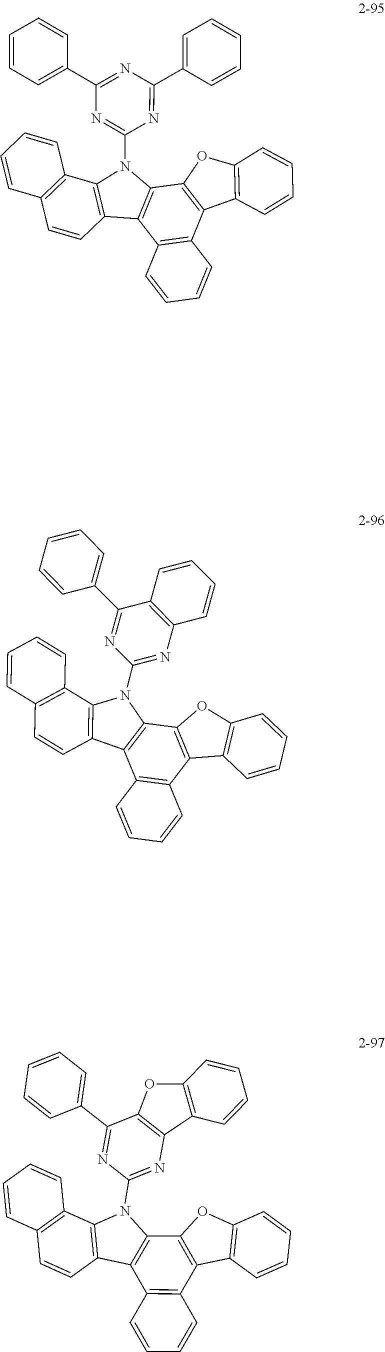 [SMILES: c1ccc(-c2nc(-c3ccccc3)nc(-n3c4c5ccccc5ccc4c4c5ccccc5c5c6ccccc6oc5c43)n2)cc1 ; c1ccc(-c2nc(-n3c4c5ccccc5ccc4c4c5ccccc5c5c6ccccc6oc5c43)nc3ccccc23)cc1 ; c1ccc(-c2nc(-n3c4c5ccccc5ccc4c4c5ccccc5c5c6ccccc6oc5c43)nc3c2oc2ccccc23)cc1]

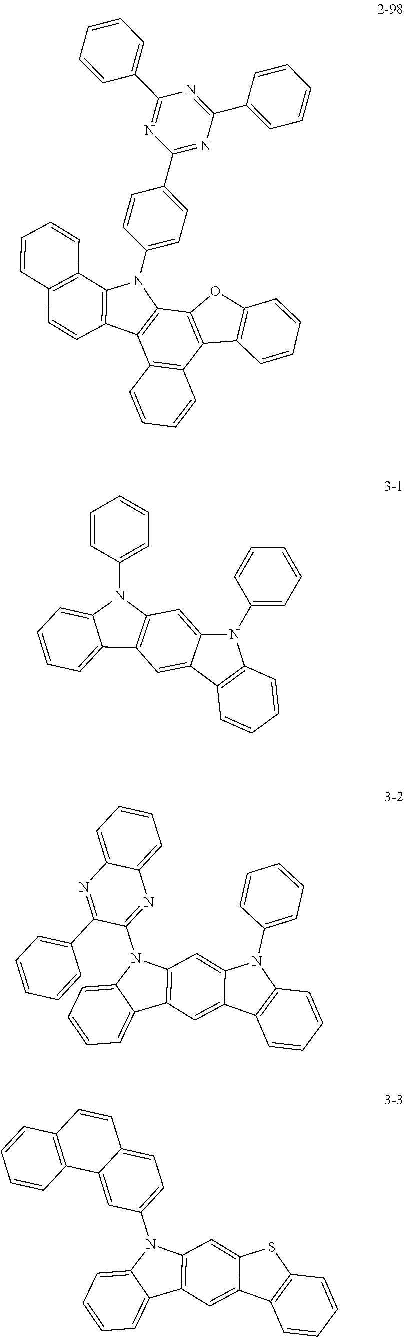 [SMILES: c1ccc(-c2nc(-c3ccccc3)nc(-c3ccc(-n4c5c6ccccc6ccc5c5c6ccccc6c6c7ccccc7oc6c54)cc3)n2)cc1 ; c1ccc(-n2c3ccccc3c3cc4c5ccccc5n(-c5ccccc5)c4cc32)cc1 ; c1ccc(-c2nc3ccccc3nc2-n2c3ccccc3c3cc4c5ccccc5n(-c5ccccc5)c4cc32)cc1 ; c1ccc2c(c1)ccc1ccc(-n3c4ccccc4c4cc5c(cc43)sc3ccccc35)cc12]

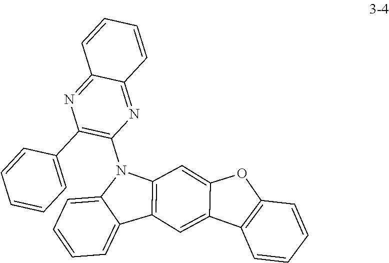 [SMILES: c1ccc(-c2nc3ccccc3nc2-n2c3ccccc3c3cc4c(cc32)oc2ccccc24)cc1]

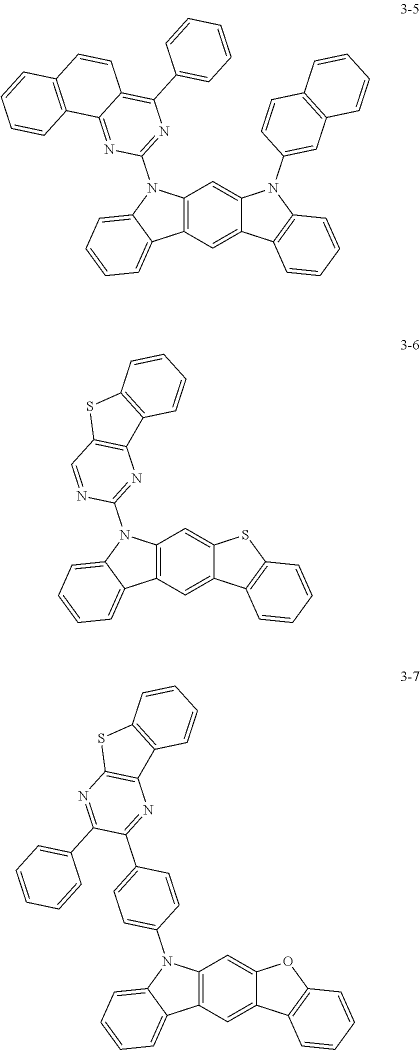 [SMILES: c1ccc(-c2nc(-n3c4ccccc4c4cc5c6ccccc6n(-c6ccc7ccccc7c6)c5cc43)nc3c2ccc2ccccc23)cc1 ; c1ccc2c(c1)sc1cc3c(cc12)c1ccccc1n3-c1ncc2sc3ccccc3c2n1 ; c1ccc(-c2nc3sc4ccccc4c3nc2-c2ccc(-n3c4ccccc4c4cc5c(cc43)oc3ccccc35)cc2)cc1]

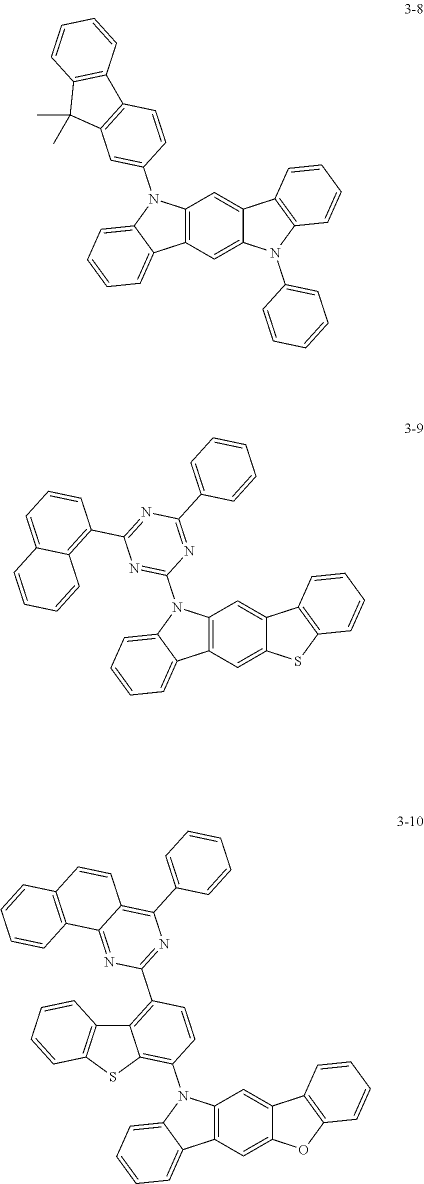 [SMILES: CC1(C)c2ccccc2-c2ccc(-n3c4ccccc4c4cc5c(cc43)c3ccccc3n5-c3ccccc3)cc21 ; c1ccc(-c2nc(-c3cccc4ccccc34)nc(-n3c4ccccc4c4cc5sc6ccccc6c5cc43)n2)cc1 ; c1ccc(-c2nc(-c3ccc(-n4c5ccccc5c5cc6oc7ccccc7c6cc54)c4sc5ccccc5c34)nc3c2ccc2ccccc23)cc1]

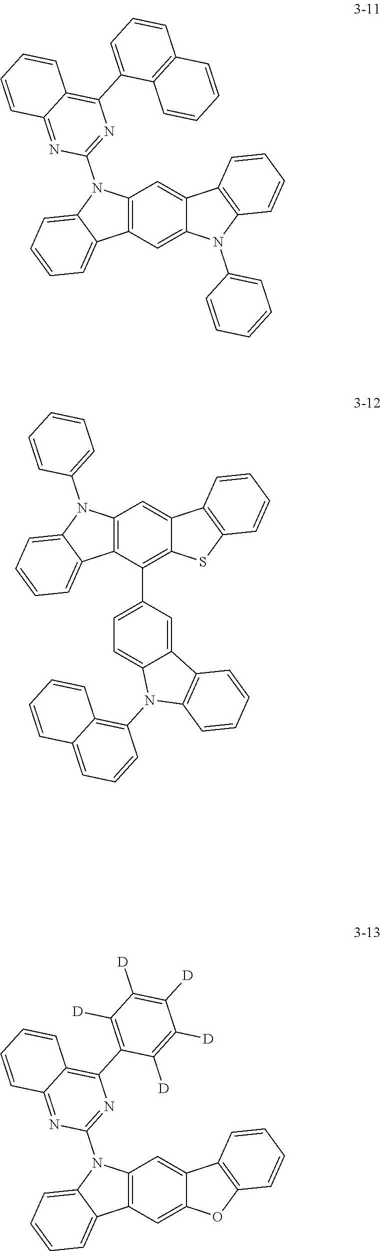 [SMILES: c1ccc(-n2c3ccccc3c3cc4c(cc32)c2ccccc2n4-c2nc(-c3cccc4ccccc34)c3ccccc3n2)cc1 ; c1ccc(-n2c3ccccc3c3c(-c4ccc5c(c4)c4ccccc4n5-c4cccc5ccccc45)c4sc5ccccc5c4cc32)cc1 ; [2H]c1c([2H])c([2H])c(-c2nc(-n3c4ccccc4c4cc5oc6ccccc6c5cc43)nc3ccccc23)c([2H])c1[2H]]

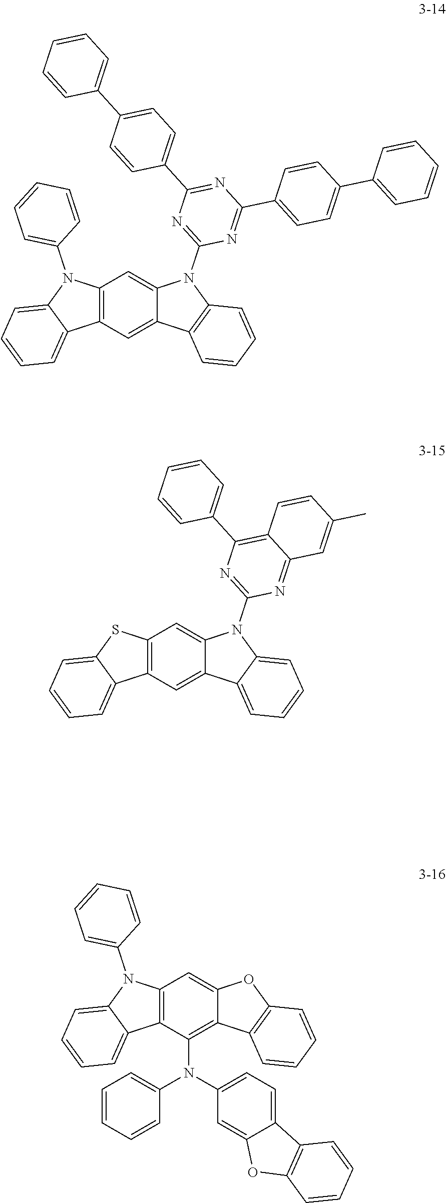 [SMILES: c1ccc(-c2ccc(-c3nc(-c4ccc(-c5ccccc5)cc4)nc(-n4c5ccccc5c5cc6c7ccccc7n(-c7ccccc7)c6cc54)n3)cc2)cc1 ; Cc1ccc2c(-c3ccccc3)nc(-n3c4ccccc4c4cc5c(cc43)sc3ccccc35)nc2c1 ; c1ccc(N(c2ccc3c(c2)oc2ccccc23)c2c3c(cc4c2c2ccccc2n4-c2ccccc2)oc2ccccc23)cc1]

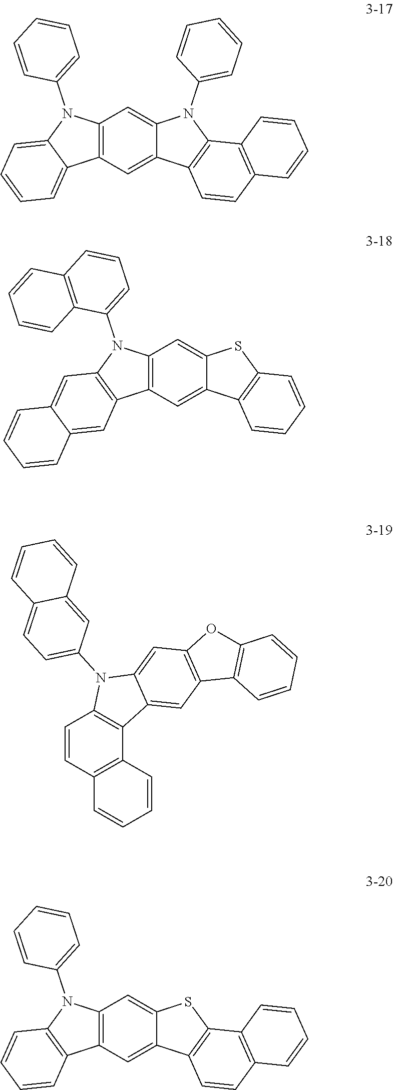 [SMILES: c1ccc(-n2c3ccccc3c3cc4c5ccc6ccccc6c5n(-c5ccccc5)c4cc32)cc1 ; c1ccc2cc3c(cc2c1)c1cc2c(cc1n3-c1cccc3ccccc13)sc1ccccc12 ; c1ccc2cc(-n3c4cc5oc6ccccc6c5cc4c4c5ccccc5ccc43)ccc2c1 ; c1ccc(-n2c3ccccc3c3cc4c(cc32)sc2c3ccccc3ccc42)cc1]

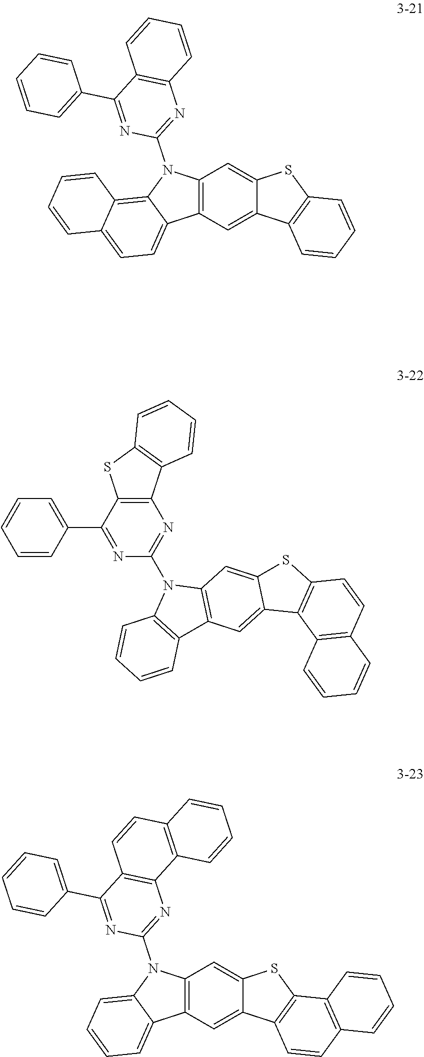 [SMILES: c1ccc(-c2nc(-n3c4cc5sc6ccccc6c5cc4c4ccc5ccccc5c43)nc3ccccc23)cc1 ; c1ccc(-c2nc(-n3c4ccccc4c4cc5c(cc43)sc3ccc4ccccc4c35)nc3c2sc2ccccc23)cc1 ; c1ccc(-c2nc(-n3c4ccccc4c4cc5c(cc43)sc3c4ccccc4ccc53)nc3c2ccc2ccccc23)cc1]

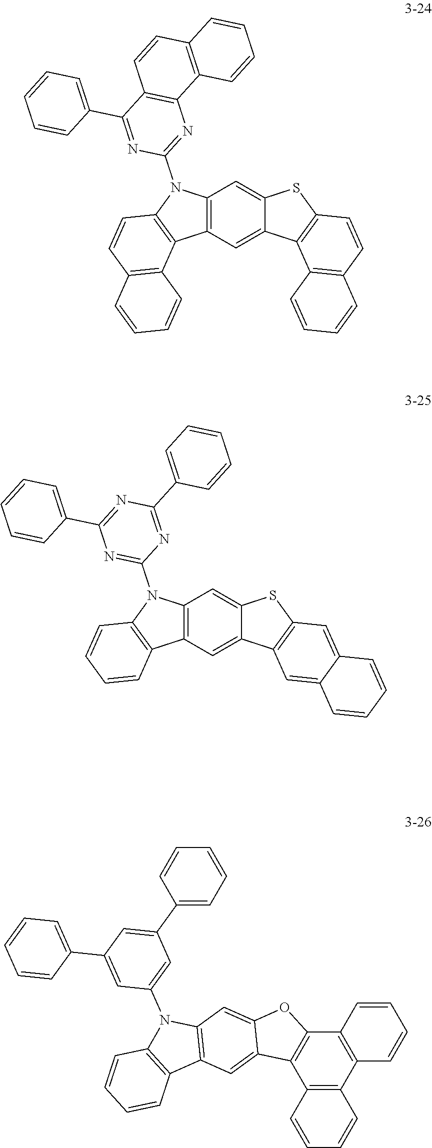 [SMILES: c1ccc(-c2nc(-n3c4cc5sc6ccc7ccccc7c6c5cc4c4c5ccccc5ccc43)nc3c2ccc2ccccc23)cc1 ; c1ccc(-c2nc(-c3ccccc3)nc(-n3c4ccccc4c4cc5c(cc43)sc3cc4ccccc4cc35)n2)cc1 ; c1ccc(-c2cc(-c3ccccc3)cc(-n3c4ccccc4c4cc5c(cc43)oc3c4ccccc4c4ccccc4c53)c2)cc1]

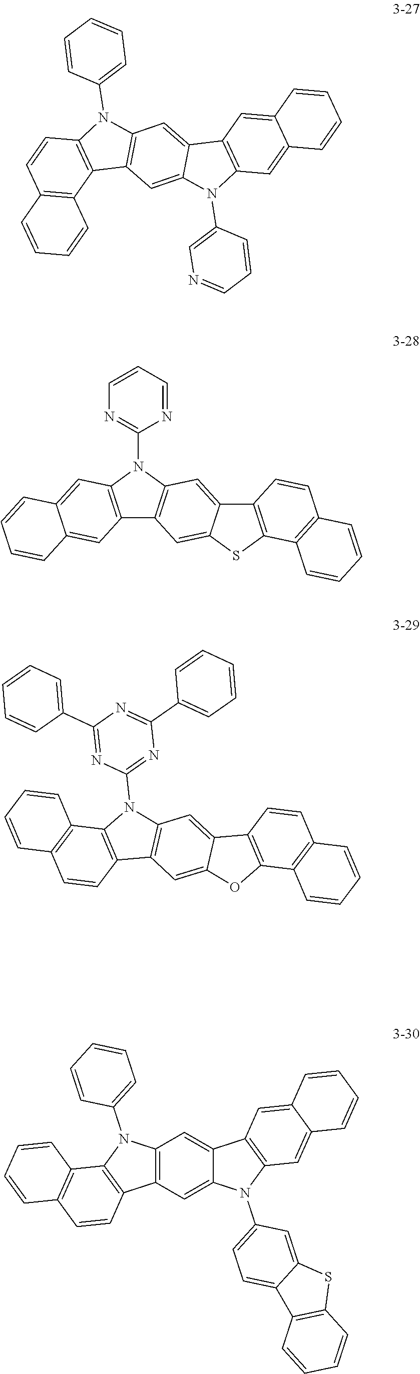 [SMILES: c1ccc(-n2c3cc4c5cc6ccccc6cc5n(-c5cccnc5)c4cc3c3c4ccccc4ccc32)cc1 ; c1cnc(-n2c3cc4ccccc4cc3c3cc4sc5c6ccccc6ccc5c4cc32)nc1 ; c1ccc(-c2nc(-c3ccccc3)nc(-n3c4cc5c(cc4c4ccc6ccccc6c43)oc3c4ccccc4ccc53)n2)cc1 ; c1ccc(-n2c3cc4c5cc6ccccc6cc5n(-c5ccc6c(c5)sc5ccccc56)c4cc3c3ccc4ccccc4c32)cc1]

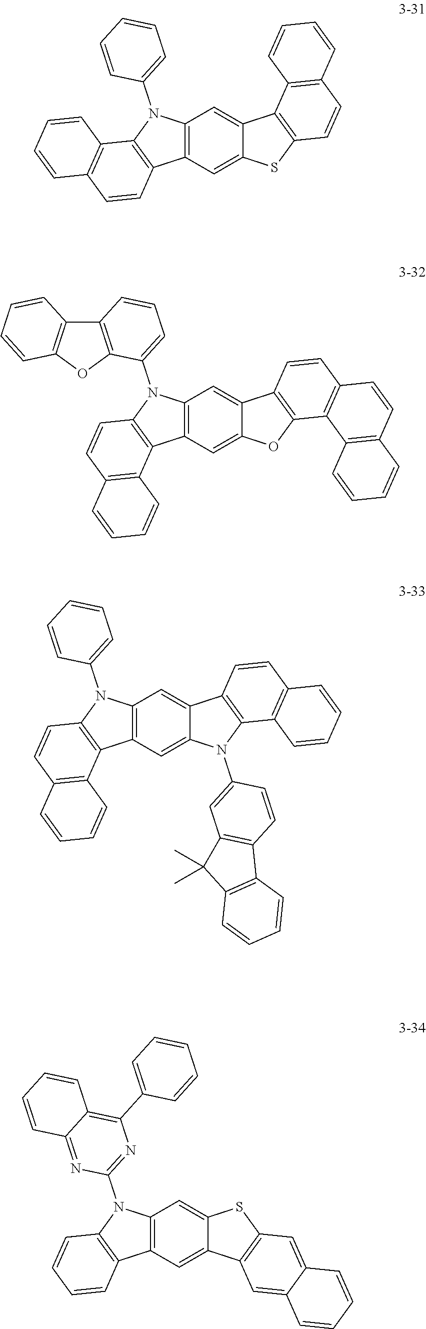 [SMILES: c1ccc(-n2c3cc4c(cc3c3ccc5ccccc5c32)sc2ccc3ccccc3c24)cc1 ; c1ccc2c(c1)ccc1ccc3c4cc5c(cc4oc3c12)c1c2ccccc2ccc1n5-c1cccc2c1oc1ccccc12 ; CC1(C)c2ccccc2-c2ccc(-n3c4cc5c6c7ccccc7ccc6n(-c6ccccc6)c5cc4c4ccc5ccccc5c43)cc21 ; c1ccc(-c2nc(-n3c4ccccc4c4cc5c(cc43)sc3cc4ccccc4cc35)nc3ccccc23)cc1]

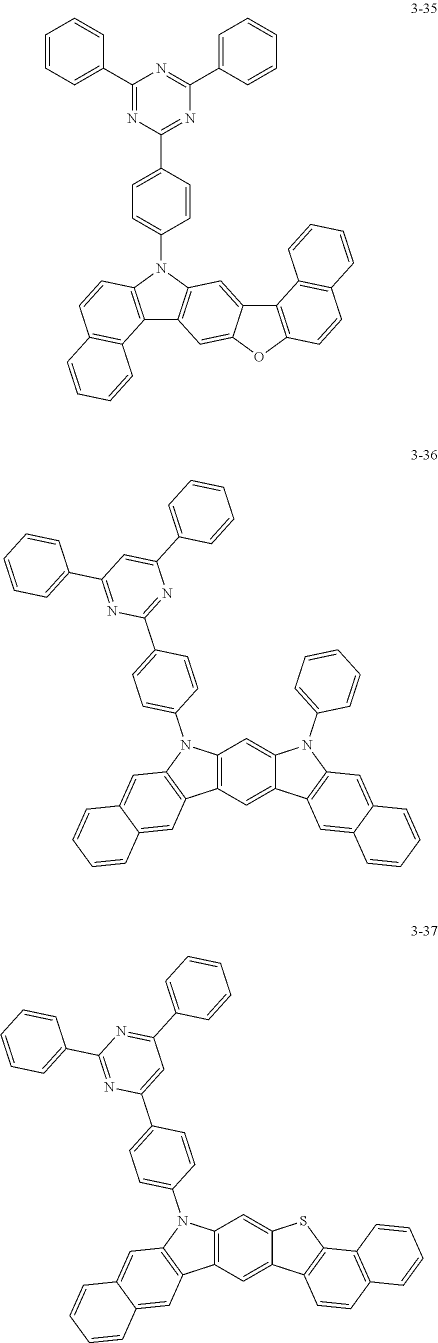 [SMILES: c1ccc(-c2nc(-c3ccccc3)nc(-c3ccc(-n4c5cc6c(cc5c5c7ccccc7ccc54)oc4ccc5ccccc5c46)cc3)n2)cc1 ; c1ccc(-c2cc(-c3ccccc3)nc(-c3ccc(-n4c5cc6ccccc6cc5c5cc6c7cc8ccccc8cc7n(-c7ccccc7)c6cc54)cc3)n2)cc1 ; c1ccc(-c2cc(-c3ccc(-n4c5cc6ccccc6cc5c5cc6c(cc54)sc4c5ccccc5ccc64)cc3)nc(-c3ccccc3)n2)cc1]

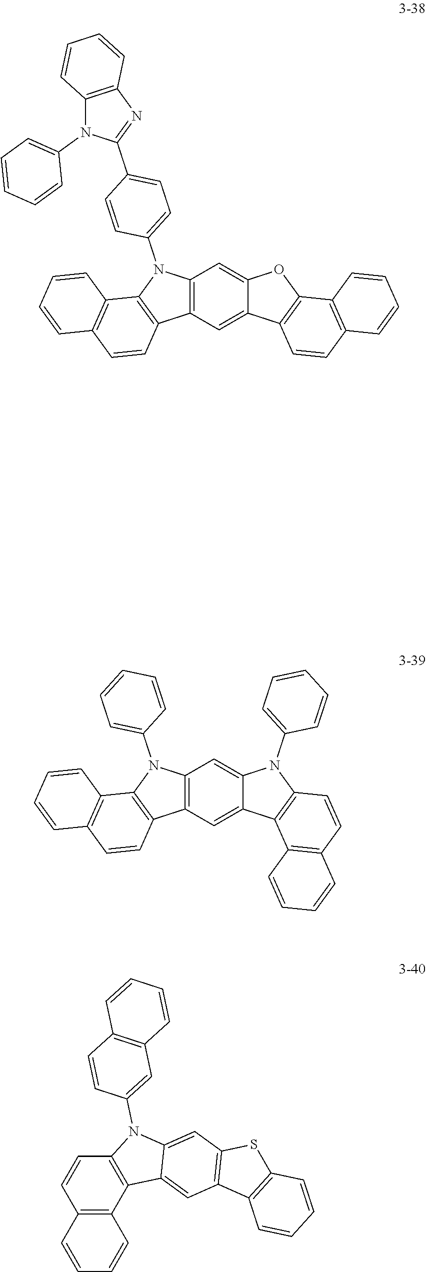 [SMILES: c1ccc(-n2c(-c3ccc(-n4c5cc6oc7c8ccccc8ccc7c6cc5c5ccc6ccccc6c54)cc3)nc3ccccc32)cc1 ; c1ccc(-n2c3cc4c(cc3c3c5ccccc5ccc32)c2ccc3ccccc3c2n4-c2ccccc2)cc1 ; c1ccc2cc(-n3c4cc5sc6ccccc6c5cc4c4c5ccccc5ccc43)ccc2c1]

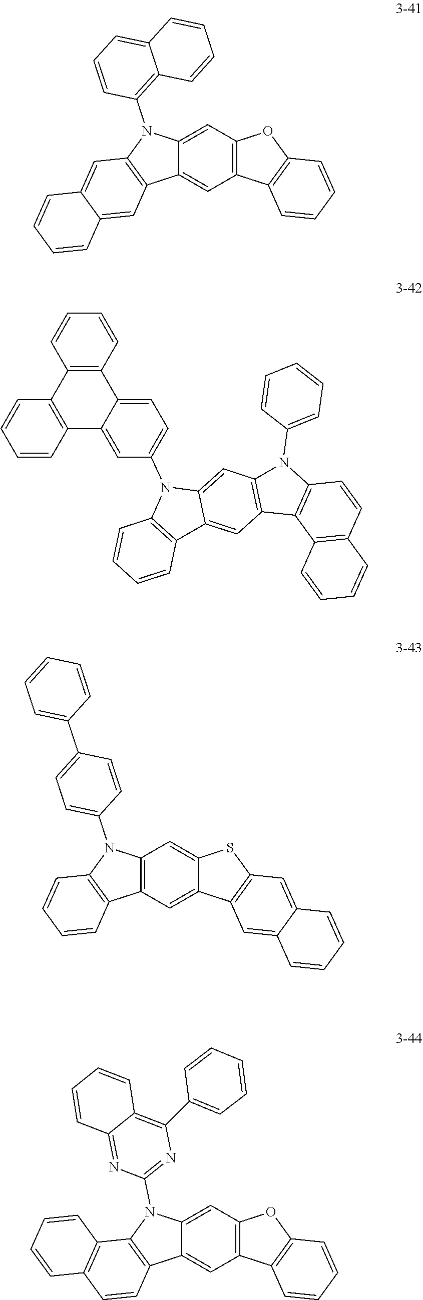 [SMILES: c1ccc2cc3c(cc2c1)c1cc2c(cc1n3-c1cccc3ccccc13)oc1ccccc12 ; c1ccc(-n2c3cc4c(cc3c3c5ccccc5ccc32)c2ccccc2n4-c2ccc3c4ccccc4c4ccccc4c3c2)cc1 ; c1ccc(-c2ccc(-n3c4ccccc4c4cc5c(cc43)sc3cc4ccccc4cc35)cc2)cc1 ; c1ccc(-c2nc(-n3c4cc5oc6ccccc6c5cc4c4ccc5ccccc5c43)nc3ccccc23)cc1]

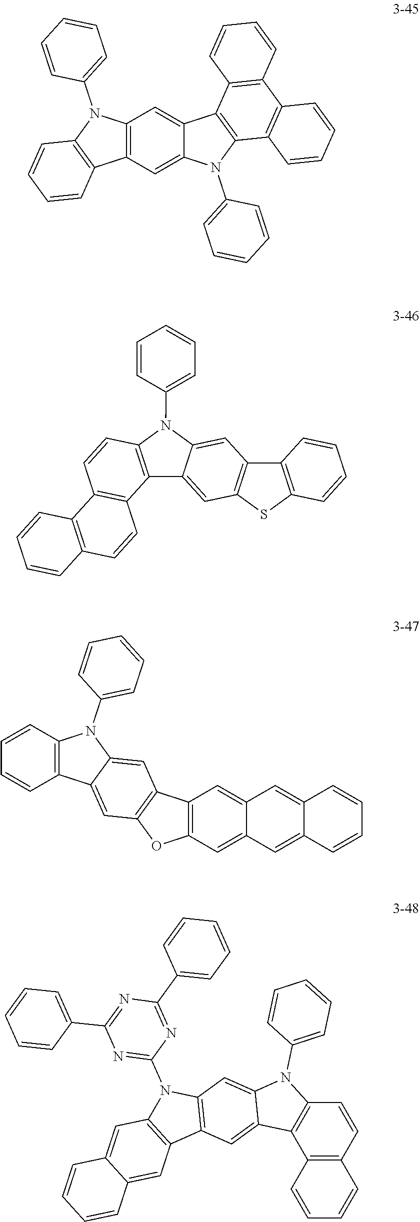 [SMILES: c1ccc(-n2c3ccccc3c3cc4c(cc32)c2c3ccccc3c3ccccc3c2n4-c2ccccc2)cc1 ; c1ccc(-n2c3cc4c(cc3c3c5ccc6ccccc6c5ccc32)sc2ccccc24)cc1 ; c1ccc(-n2c3ccccc3c3cc4oc5cc6cc7ccccc7cc6cc5c4cc32)cc1 ; c1ccc(-c2nc(-c3ccccc3)nc(-n3c4cc5ccccc5cc4c4cc5c6c7ccccc7ccc6n(-c6ccccc6)c5cc43)n2)cc1]

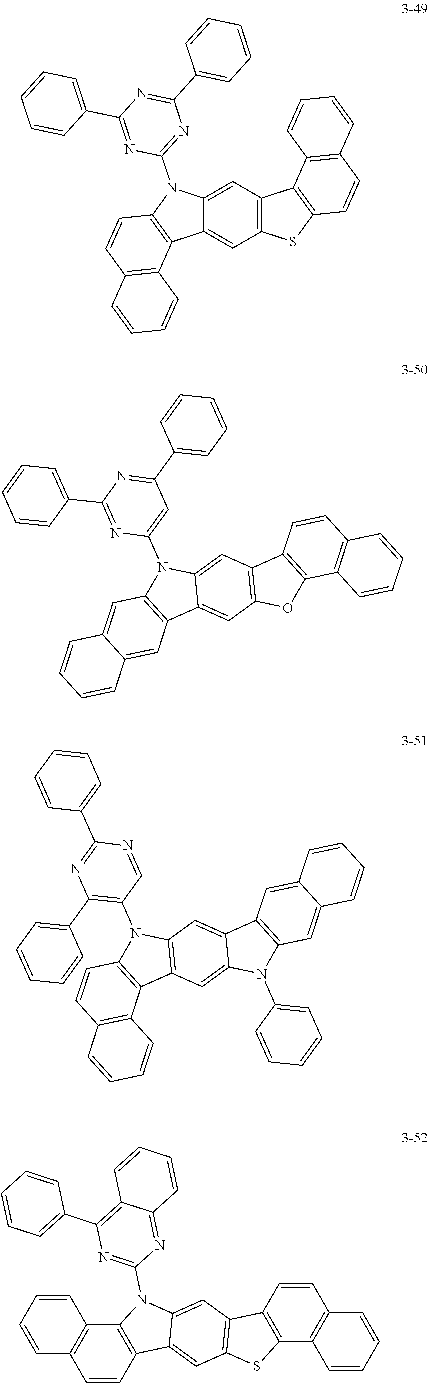 [SMILES: c1ccc(-c2nc(-c3ccccc3)nc(-n3c4cc5c(cc4c4c6ccccc6ccc43)sc3ccc4ccccc4c35)n2)cc1 ; c1ccc(-c2cc(-n3c4cc5ccccc5cc4c4cc5oc6c7ccccc7ccc6c5cc43)nc(-c3ccccc3)n2)cc1 ; c1ccc(-c2ncc(-n3c4cc5c6cc7ccccc7cc6n(-c6ccccc6)c5cc4c4c5ccccc5ccc43)c(-c3ccccc3)n2)cc1 ; c1ccc(-c2nc(-n3c4cc5c(cc4c4ccc6ccccc6c43)sc3c4ccccc4ccc53)nc3ccccc23)cc1]

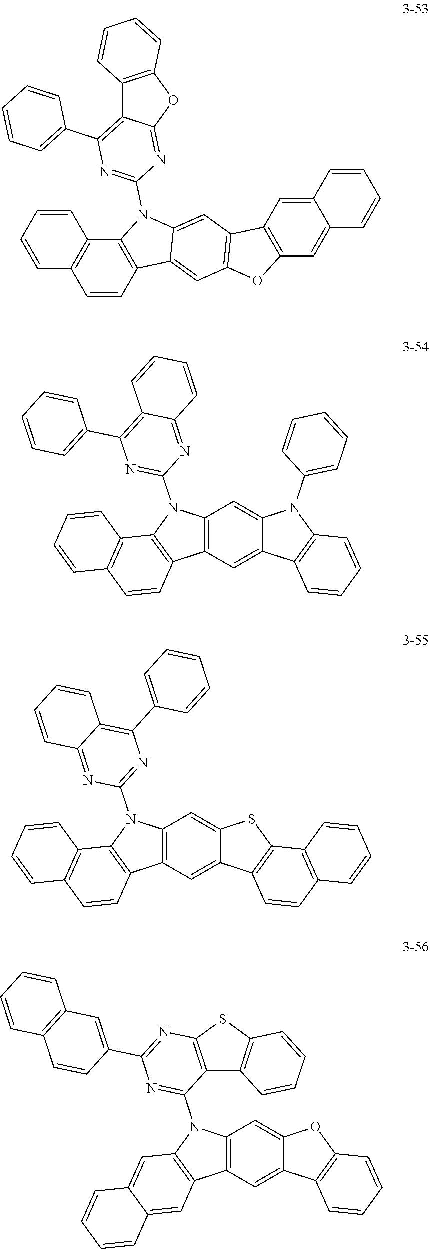 [SMILES: c1ccc(-c2nc(-n3c4cc5c(cc4c4ccc6ccccc6c43)oc3cc4ccccc4cc35)nc3oc4ccccc4c23)cc1 ; c1ccc(-c2nc(-n3c4cc5c(cc4c4ccc6ccccc6c43)c3ccccc3n5-c3ccccc3)nc3ccccc23)cc1 ; c1ccc(-c2nc(-n3c4cc5sc6c7ccccc7ccc6c5cc4c4ccc5ccccc5c43)nc3ccccc23)cc1 ; c1ccc2cc(-c3nc(-n4c5cc6ccccc6cc5c5cc6c(cc54)oc4ccccc46)c4c(n3)sc3ccccc34)ccc2c1]

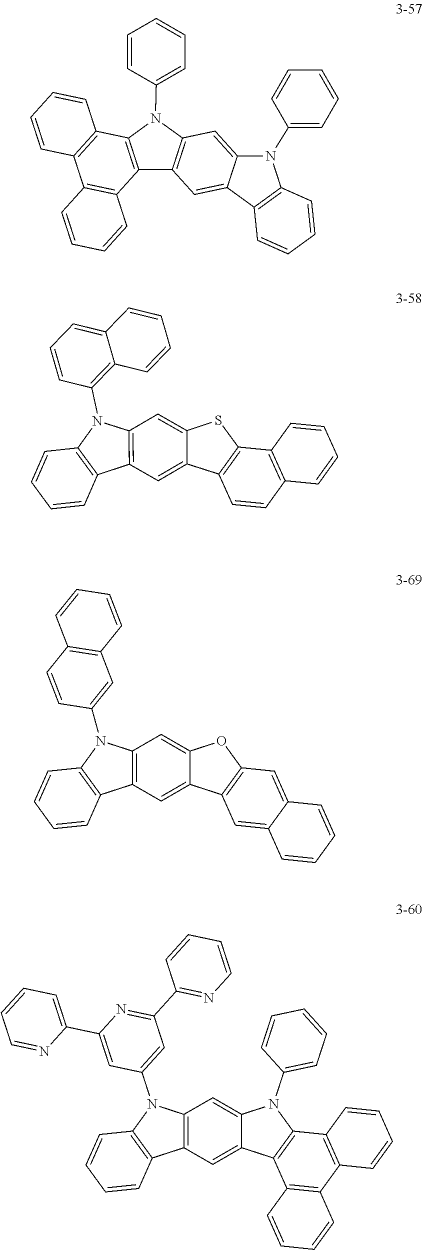 [SMILES: c1ccc(-n2c3ccccc3c3cc4c5c6ccccc6c6ccccc6c5n(-c5ccccc5)c4cc32)cc1 ; c1ccc2c(-n3c4ccccc4c4cc5c(cc43)sc3c4ccccc4ccc53)cccc2c1 ; c1ccc2cc(-n3c4ccccc4c4cc5c(cc43)oc3cc4ccccc4cc35)ccc2c1 ; c1ccc(-n2c3cc4c(cc3c3c5ccccc5c5ccccc5c32)c2ccccc2n4-c2cc(-c3ccccn3)nc(-c3ccccn3)c2)cc1]

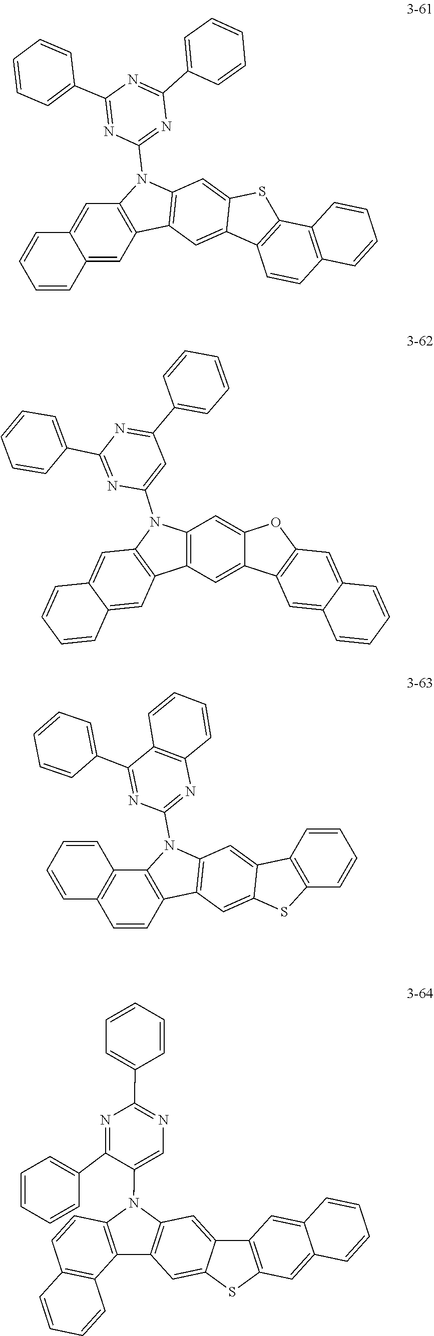 [SMILES: c1ccc(-c2nc(-c3ccccc3)nc(-n3c4cc5ccccc5cc4c4cc5c(cc43)sc3c4ccccc4ccc53)n2)cc1 ; c1ccc(-c2cc(-n3c4cc5ccccc5cc4c4cc5c(cc43)oc3cc4ccccc4cc35)nc(-c3ccccc3)n2)cc1 ; c1ccc(-c2nc(-n3c4cc5c(cc4c4ccc6ccccc6c43)sc3ccccc35)nc3ccccc23)cc1 ; c1ccc(-c2ncc(-n3c4cc5c(cc4c4c6ccccc6ccc43)sc3cc4ccccc4cc35)c(-c3ccccc3)n2)cc1]

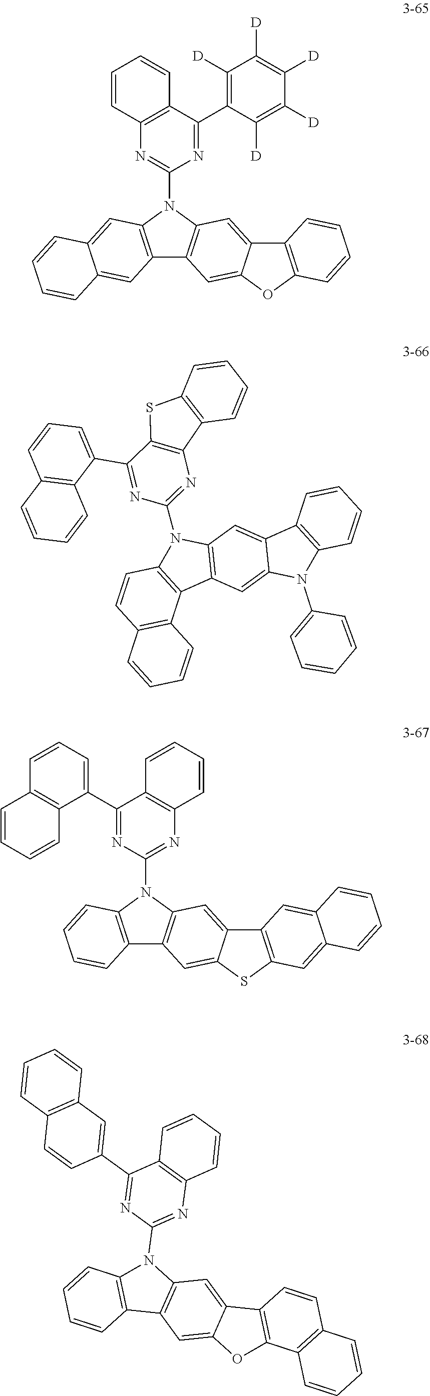 [SMILES: [2H]c1c([2H])c([2H])c(-c2nc(-n3c4cc5ccccc5cc4c4cc5oc6ccccc6c5cc43)nc3ccccc23)c([2H])c1[2H] ; c1ccc(-n2c3ccccc3c3cc4c(cc32)c2c3ccccc3ccc2n4-c2nc(-c3cccc4ccccc34)c3sc4ccccc4c3n2)cc1 ; c1ccc2cc3c(cc2c1)sc1cc2c4ccccc4n(-c4nc(-c5cccc6ccccc56)c5ccccc5n4)c2cc13 ; c1ccc2cc(-c3nc(-n4c5ccccc5c5cc6oc7c8ccccc8ccc7c6cc54)nc4ccccc34)ccc2c1]

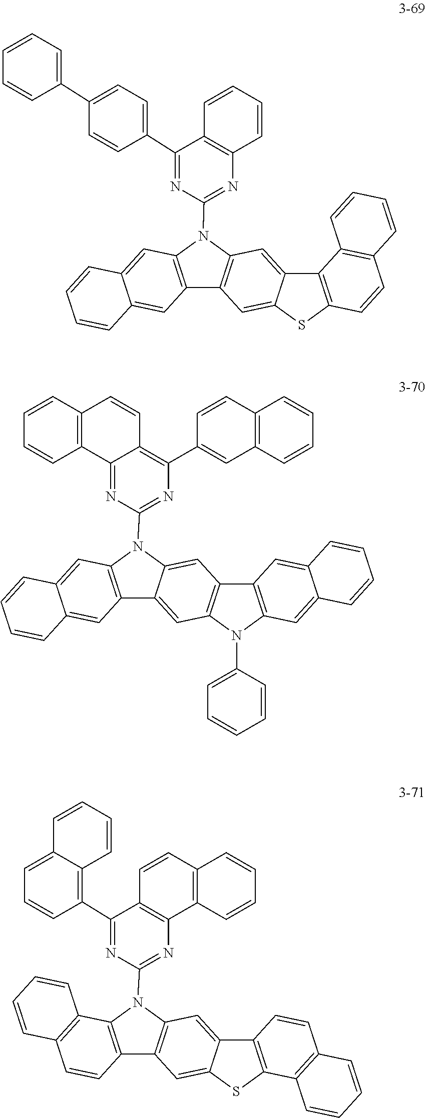 [SMILES: c1ccc(-c2ccc(-c3nc(-n4c5cc6ccccc6cc5c5cc6sc7ccc8ccccc8c7c6cc54)nc4ccccc34)cc2)cc1 ; c1ccc(-n2c3cc4ccccc4cc3c3cc4c(cc32)c2cc3ccccc3cc2n4-c2nc(-c3ccc4ccccc4c3)c3ccc4ccccc4c3n2)cc1 ; c1ccc2c(-c3nc(-n4c5cc6c(cc5c5ccc7ccccc7c54)sc4c5ccccc5ccc64)nc4c3ccc3ccccc34)cccc2c1]

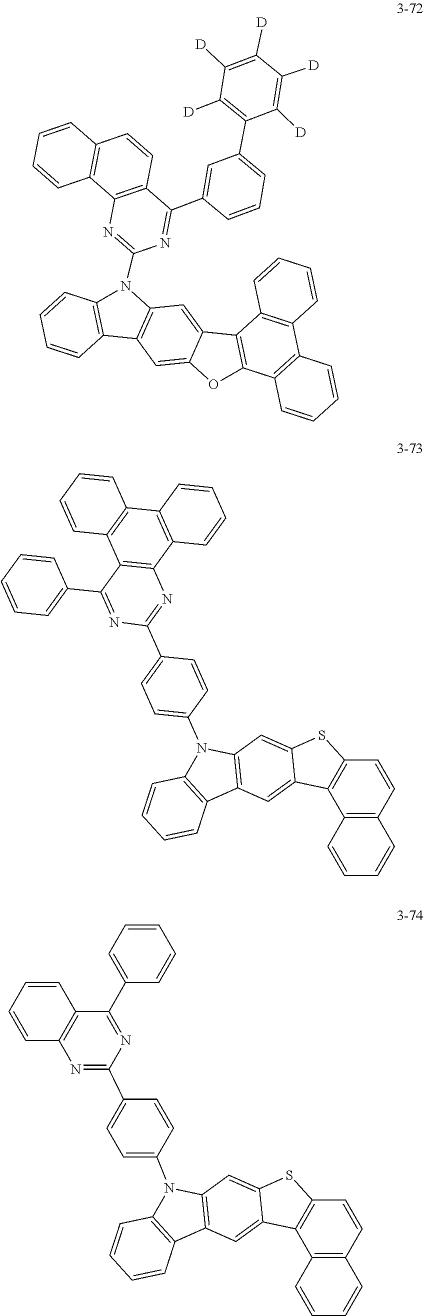 [SMILES: [2H]c1c([2H])c([2H])c(-c2cccc(-c3nc(-n4c5ccccc5c5cc6oc7c8ccccc8c8ccccc8c7c6cc54)nc4c3ccc3ccccc34)c2)c([2H])c1[2H] ; c1ccc(-c2nc(-c3ccc(-n4c5ccccc5c5cc6c(cc54)sc4ccc5ccccc5c46)cc3)nc3c4ccccc4c4ccccc4c23)cc1 ; c1ccc(-c2nc(-c3ccc(-n4c5ccccc5c5cc6c(cc54)sc4ccc5ccccc5c46)cc3)nc3ccccc23)cc1]

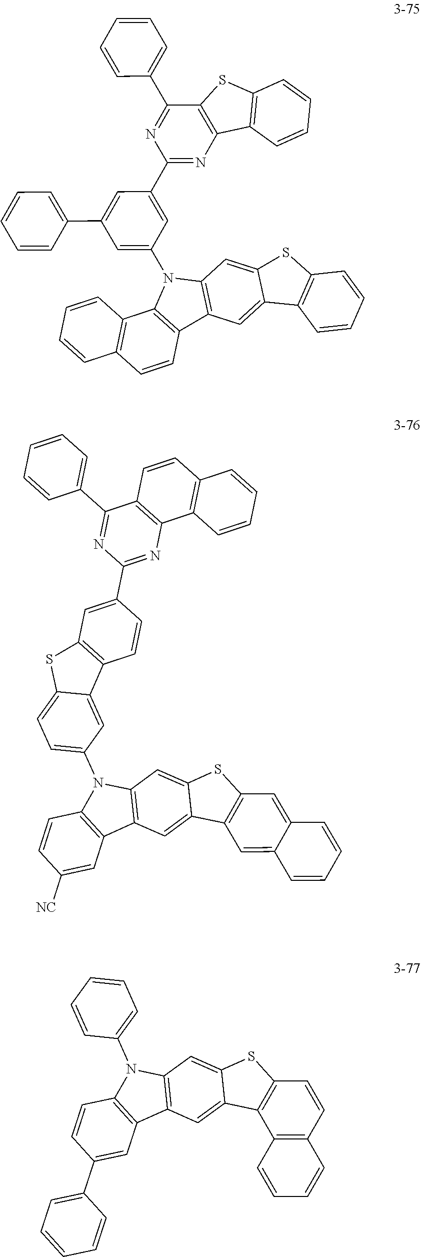 [SMILES: c1ccc(-c2cc(-c3nc(-c4ccccc4)c4sc5ccccc5c4n3)cc(-n3c4cc5sc6ccccc6c5cc4c4ccc5ccccc5c43)c2)cc1 ; N#Cc1ccc2c(c1)c1cc3c(cc1n2-c1ccc2sc4cc(-c5nc(-c6ccccc6)c6ccc7ccccc7c6n5)ccc4c2c1)sc1cc2ccccc2cc13 ; c1ccc(-c2ccc3c(c2)c2cc4c(cc2n3-c2ccccc2)sc2ccc3ccccc3c24)cc1]

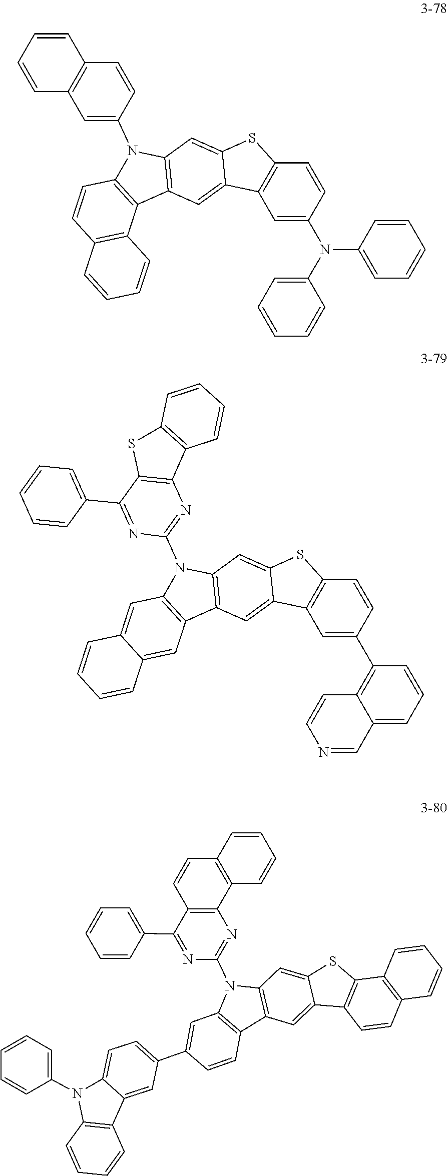 [SMILES: c1ccc(N(c2ccccc2)c2ccc3sc4cc5c(cc4c3c2)c2c3ccccc3ccc2n5-c2ccc3ccccc3c2)cc1 ; c1ccc(-c2nc(-n3c4cc5ccccc5cc4c4cc5c(cc43)sc3ccc(-c4cccc6cnccc46)cc35)nc3c2sc2ccccc23)cc1 ; c1ccc(-c2nc(-n3c4cc(-c5ccc6c(c5)c5ccccc5n6-c5ccccc5)ccc4c4cc5c(cc43)sc3c4ccccc4ccc53)nc3c2ccc2ccccc23)cc1]

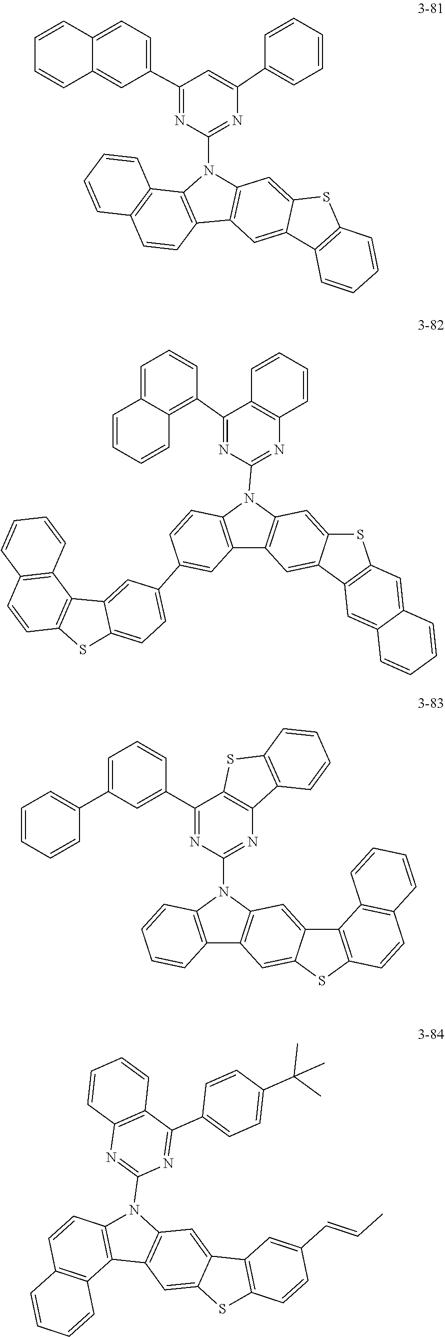 [SMILES: c1ccc(-c2cc(-c3ccc4ccccc4c3)nc(-n3c4cc5sc6ccccc6c5cc4c4ccc5ccccc5c43)n2)cc1 ; c1ccc2cc3c(cc2c1)sc1cc2c(cc13)c1cc(-c3ccc4sc5ccc6ccccc6c5c4c3)ccc1n2-c1nc(-c2cccc3ccccc23)c2ccccc2n1 ; c1ccc(-c2cccc(-c3nc(-n4c5ccccc5c5cc6sc7ccc8ccccc8c7c6cc54)nc4c3sc3ccccc34)c2)cc1 ; C/C=C/c1ccc2sc3cc4c5c6ccccc6ccc5n(-c5nc(-c6ccc(C(C)(C)C)cc6)c6ccccc6n5)c4cc3c2c1]

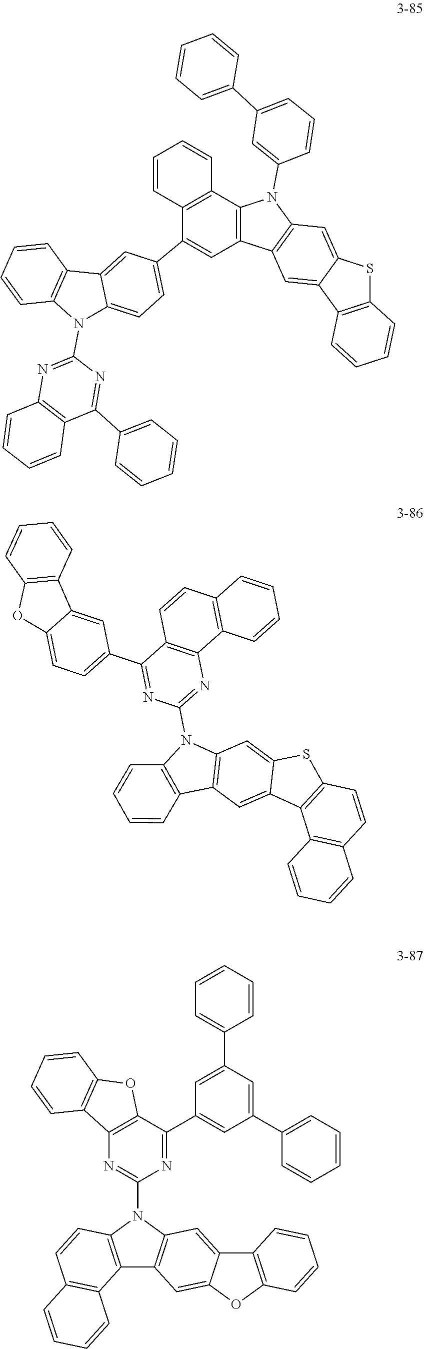 [SMILES: c1ccc(-c2cccc(-n3c4cc5sc6ccccc6c5cc4c4cc(-c5ccc6c(c5)c5ccccc5n6-c5nc(-c6ccccc6)c6ccccc6n5)c5ccccc5c43)c2)cc1 ; c1ccc2c(c1)ccc1c(-c3ccc4oc5ccccc5c4c3)nc(-n3c4ccccc4c4cc5c(cc43)sc3ccc4ccccc4c35)nc12 ; c1ccc(-c2cc(-c3ccccc3)cc(-c3nc(-n4c5cc6c(cc5c5c7ccccc7ccc54)oc4ccccc46)nc4c3oc3ccccc34)c2)cc1]

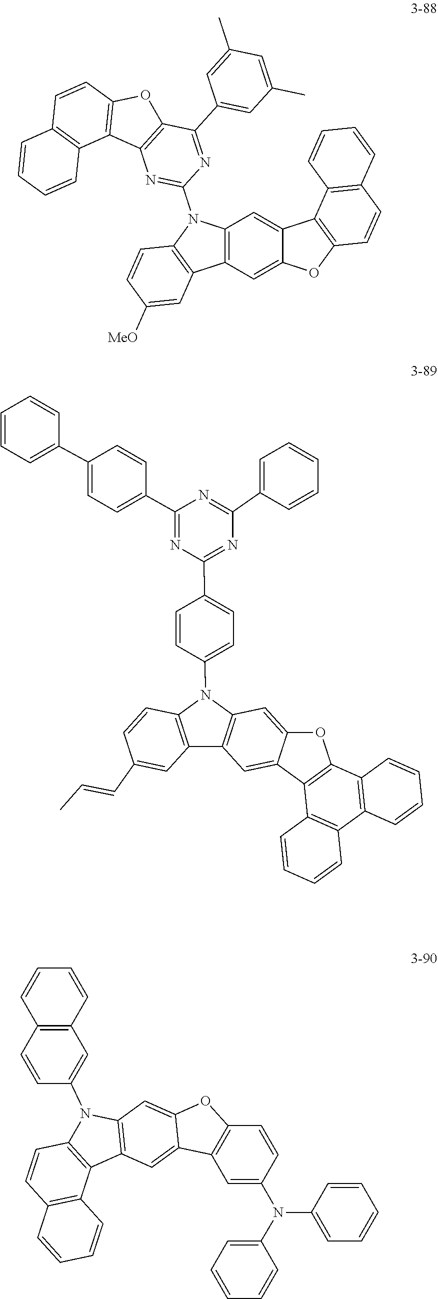 [SMILES: COc1ccc2c(c1)c1cc3oc4ccc5ccccc5c4c3cc1n2-c1nc(-c2cc(C)cc(C)c2)c2oc3ccc4ccccc4c3c2n1 ; C/C=C/c1ccc2c(c1)c1cc3c(cc1n2-c1ccc(-c2nc(-c4ccccc4)nc(-c4ccc(-c5ccccc5)cc4)n2)cc1)oc1c2ccccc2c2ccccc2c31 ; c1ccc(N(c2ccccc2)c2ccc3oc4cc5c(cc4c3c2)c2c3ccccc3ccc2n5-c2ccc3ccccc3c2)cc1]

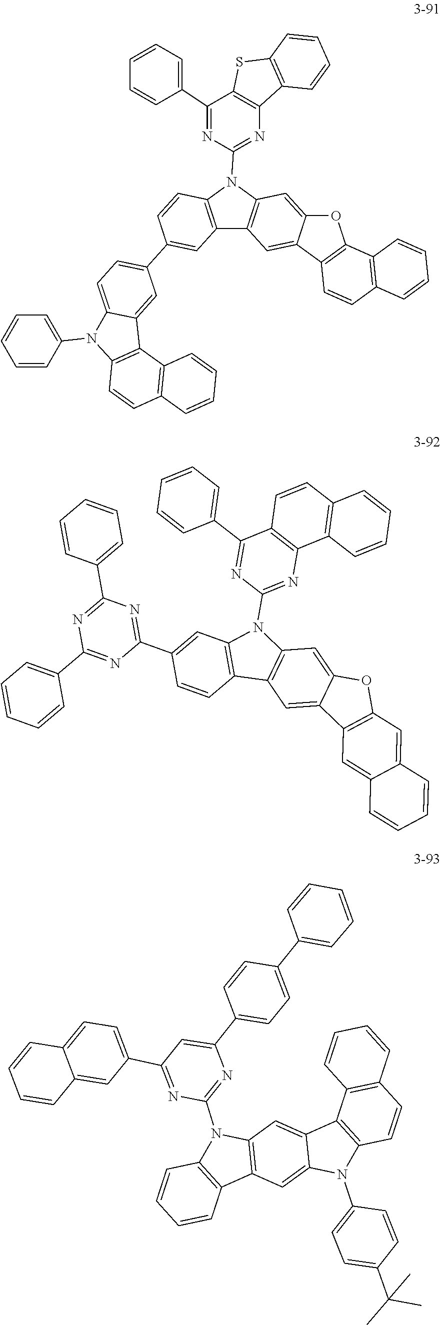 [SMILES: c1ccc(-c2nc(-n3c4ccc(-c5ccc6c(c5)c5c7ccccc7ccc5n6-c5ccccc5)cc4c4cc5c(cc43)oc3c4ccccc4ccc53)nc3c2sc2ccccc23)cc1 ; c1ccc(-c2nc(-c3ccccc3)nc(-c3ccc4c5cc6c(cc5n(-c5nc(-c7ccccc7)c7ccc8ccccc8c7n5)c4c3)oc3cc4ccccc4cc36)n2)cc1 ; CC(C)(C)c1ccc(-n2c3cc4c5ccccc5n(-c5nc(-c6ccc(-c7ccccc7)cc6)cc(-c6ccc7ccccc7c6)n5)c4cc3c3c4ccccc4ccc32)cc1]

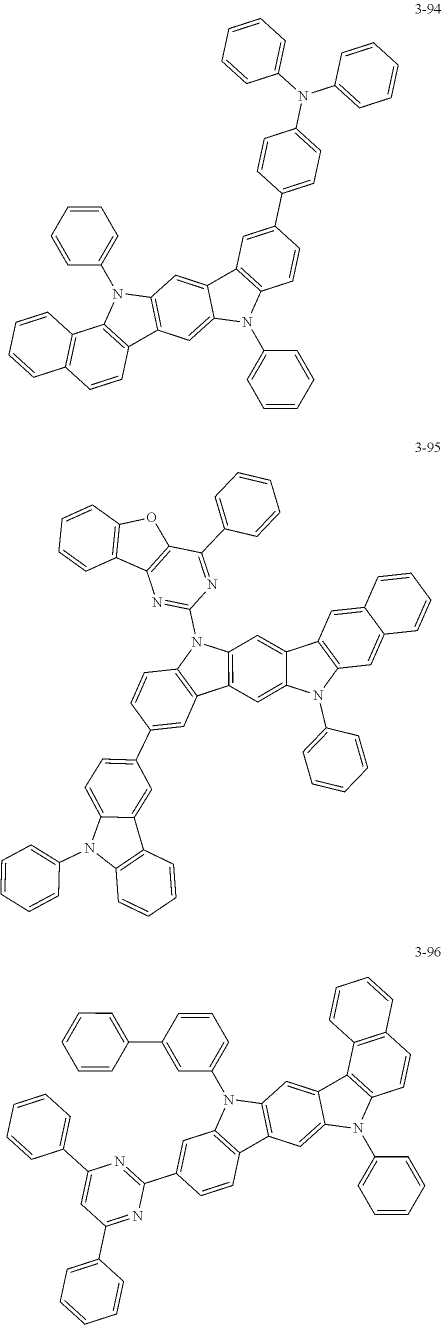 [SMILES: c1ccc(N(c2ccccc2)c2ccc(-c3ccc4c(c3)c3cc5c(cc3n4-c3ccccc3)c3ccc4ccccc4c3n5-c3ccccc3)cc2)cc1 ; c1ccc(-c2nc(-n3c4ccc(-c5ccc6c(c5)c5ccccc5n6-c5ccccc5)cc4c4cc5c(cc43)c3cc4ccccc4cc3n5-c3ccccc3)nc3c2oc2ccccc23)cc1 ; c1ccc(-c2cccc(-n3c4cc(-c5nc(-c6ccccc6)cc(-c6ccccc6)n5)ccc4c4cc5c(cc43)c3c4ccccc4ccc3n5-c3ccccc3)c2)cc1]

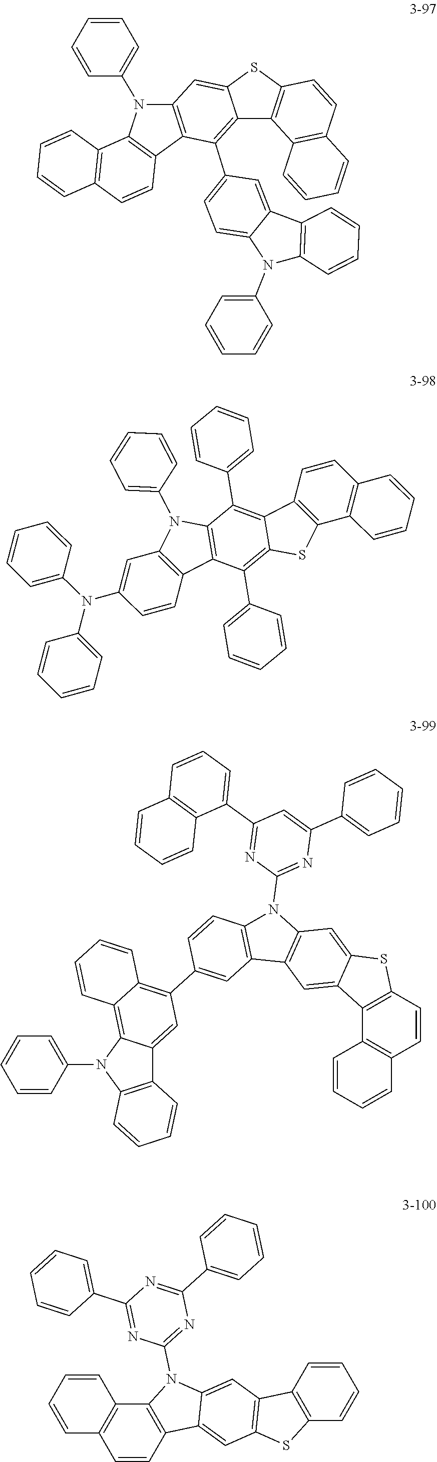 [SMILES: c1ccc(-n2c3ccccc3c3cc(-c4c5c(cc6c4c4ccc7ccccc7c4n6-c4ccccc4)sc4ccc6ccccc6c45)ccc32)cc1 ; c1ccc(-c2c3sc4c5ccccc5ccc4c3c(-c3ccccc3)c3c2c2ccc(N(c4ccccc4)c4ccccc4)cc2n3-c2ccccc2)cc1 ; c1ccc(-c2cc(-c3cccc4ccccc34)nc(-n3c4ccc(-c5cc6c7ccccc7n(-c7ccccc7)c6c6ccccc56)cc4c4cc5c(cc43)sc3ccc4ccccc4c35)n2)cc1 ; c1ccc(-c2nc(-c3ccccc3)nc(-n3c4cc5c(cc4c4ccc6ccccc6c43)sc3ccccc35)n2)cc1]

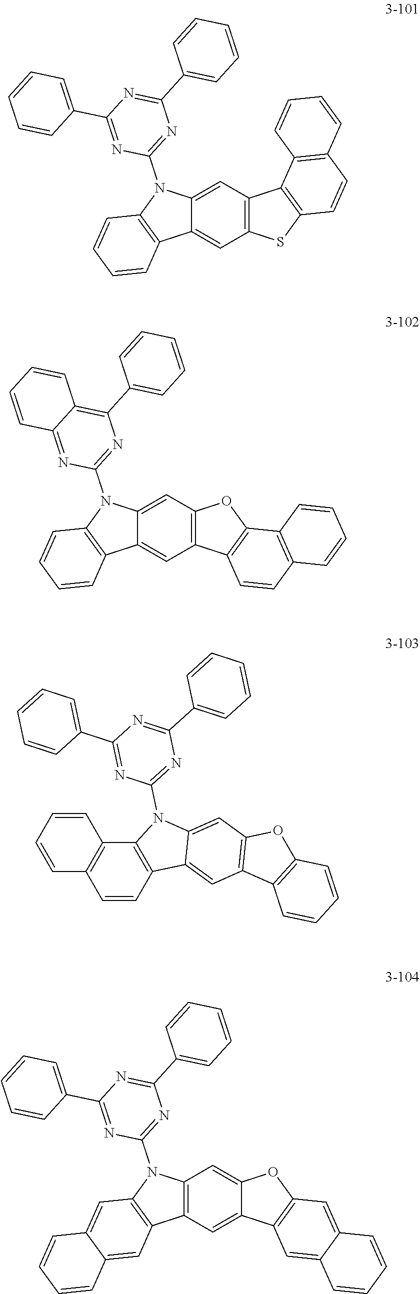 [SMILES: c1ccc(-c2nc(-c3ccccc3)nc(-n3c4ccccc4c4cc5sc6ccc7ccccc7c6c5cc43)n2)cc1 ; c1ccc(-c2nc(-n3c4ccccc4c4cc5c(cc43)oc3c4ccccc4ccc53)nc3ccccc23)cc1 ; c1ccc(-c2nc(-c3ccccc3)nc(-n3c4cc5oc6ccccc6c5cc4c4ccc5ccccc5c43)n2)cc1 ; c1ccc(-c2nc(-c3ccccc3)nc(-n3c4cc5ccccc5cc4c4cc5c(cc43)oc3cc4ccccc4cc35)n2)cc1]

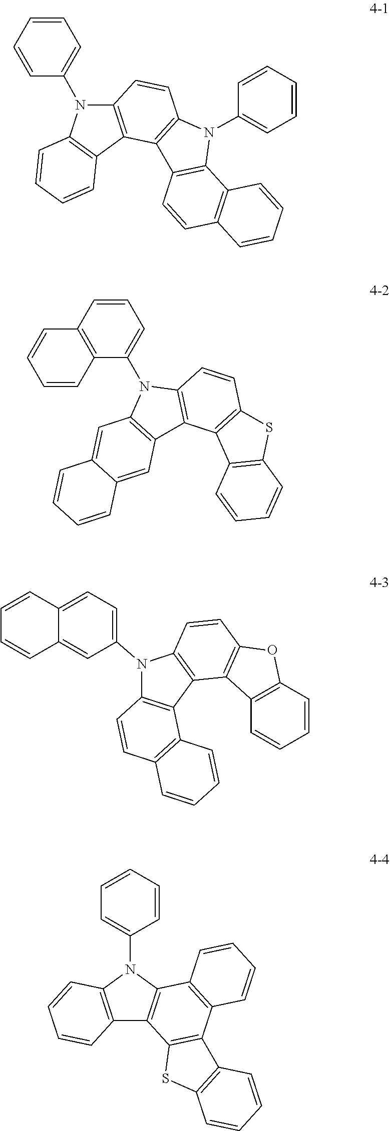 [SMILES: c1ccc(-n2c3ccccc3c3c4c5ccc6ccccc6c5n(-c5ccccc5)c4ccc32)cc1 ; c1ccc2cc3c(cc2c1)c1c2c(ccc1n3-c1cccc3ccccc13)sc1ccccc12 ; c1ccc2cc(-n3c4ccc5ccccc5c4c4c5c(ccc43)oc3ccccc35)ccc2c1 ; c1ccc(-n2c3ccccc3c3c4sc5ccccc5c4c4ccccc4c32)cc1]

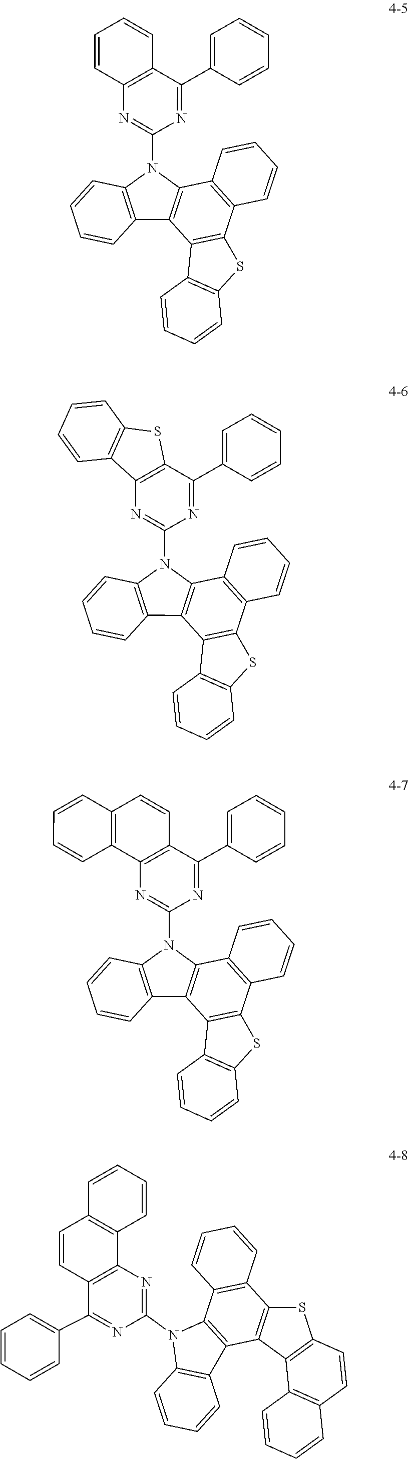 [SMILES: c1ccc(-c2nc(-n3c4ccccc4c4c5c6ccccc6sc5c5ccccc5c43)nc3ccccc23)cc1 ; c1ccc(-c2nc(-n3c4ccccc4c4c5c6ccccc6sc5c5ccccc5c43)nc3c2sc2ccccc23)cc1 ; c1ccc(-c2nc(-n3c4ccccc4c4c5c6ccccc6sc5c5ccccc5c43)nc3c2ccc2ccccc23)cc1 ; c1ccc(-c2nc(-n3c4ccccc4c4c5c(sc6ccc7ccccc7c65)c5ccccc5c43)nc3c2ccc2ccccc23)cc1]

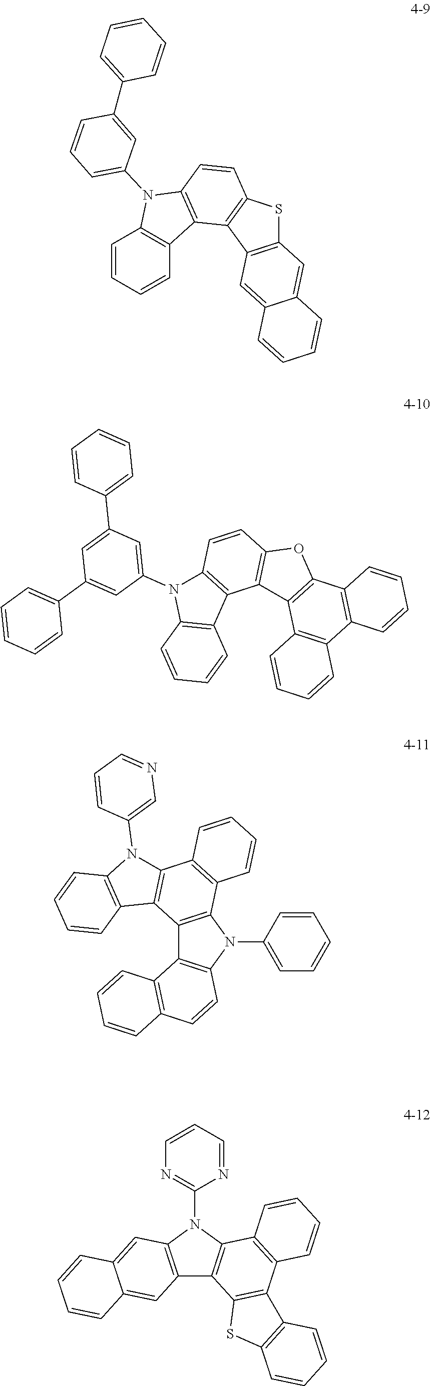 [SMILES: c1ccc(-c2cccc(-n3c4ccccc4c4c5c(ccc43)sc3cc4ccccc4cc35)c2)cc1 ; c1ccc(-c2cc(-c3ccccc3)cc(-n3c4ccccc4c4c5c(ccc43)oc3c4ccccc4c4ccccc4c35)c2)cc1 ; c1ccc(-n2c3ccc4ccccc4c3c3c4c5ccccc5n(-c5cccnc5)c4c4ccccc4c32)cc1 ; c1cnc(-n2c3cc4ccccc4cc3c3c4sc5ccccc5c4c4ccccc4c32)nc1]

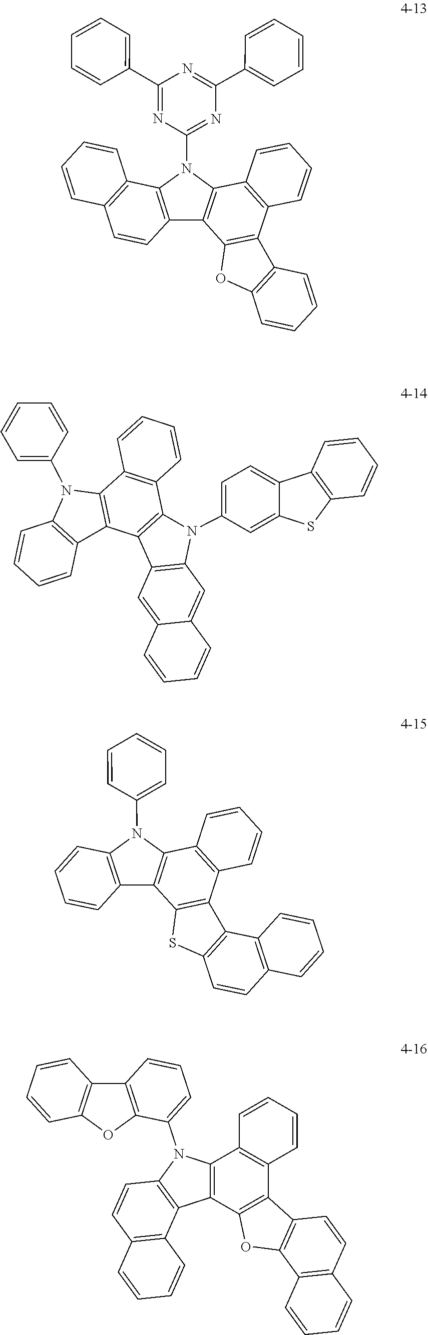 [SMILES: c1ccc(-c2nc(-c3ccccc3)nc(-n3c4c5ccccc5ccc4c4c5oc6ccccc6c5c5ccccc5c43)n2)cc1 ; c1ccc(-n2c3ccccc3c3c4c5cc6ccccc6cc5n(-c5ccc6c(c5)sc5ccccc56)c4c4ccccc4c32)cc1 ; c1ccc(-n2c3ccccc3c3c4sc5ccc6ccccc6c5c4c4ccccc4c32)cc1 ; c1ccc2c(c1)ccc1c2oc2c1c1ccccc1c1c2c2c3ccccc3ccc2n1-c1cccc2c1oc1ccccc12]

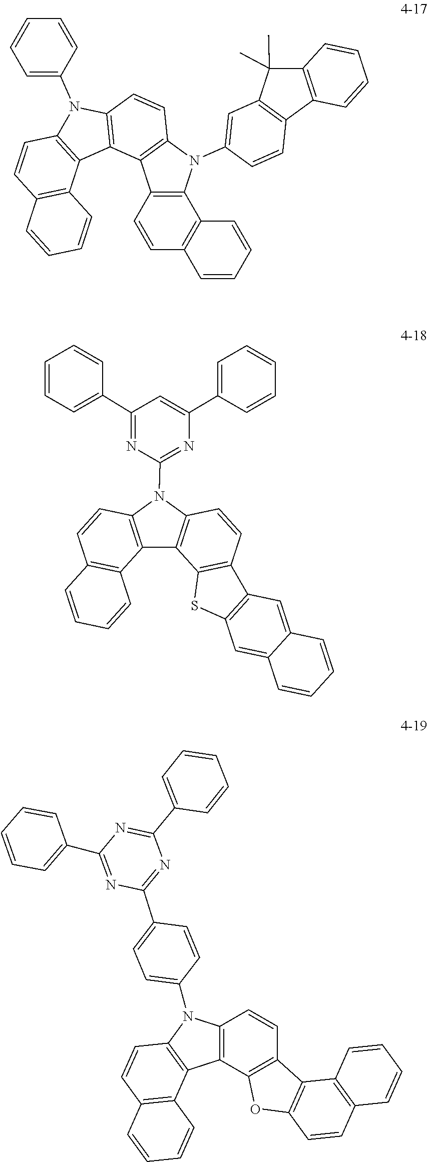 [SMILES: CC1(C)c2ccccc2-c2ccc(-n3c4ccc5c(c6c7ccccc7ccc6n5-c5ccccc5)c4c4ccc5ccccc5c43)cc21 ; c1ccc(-c2cc(-c3ccccc3)nc(-n3c4ccc5ccccc5c4c4c5sc6cc7ccccc7cc6c5ccc43)n2)cc1 ; c1ccc(-c2nc(-c3ccccc3)nc(-c3ccc(-n4c5ccc6ccccc6c5c5c6oc7ccc8ccccc8c7c6ccc54)cc3)n2)cc1]

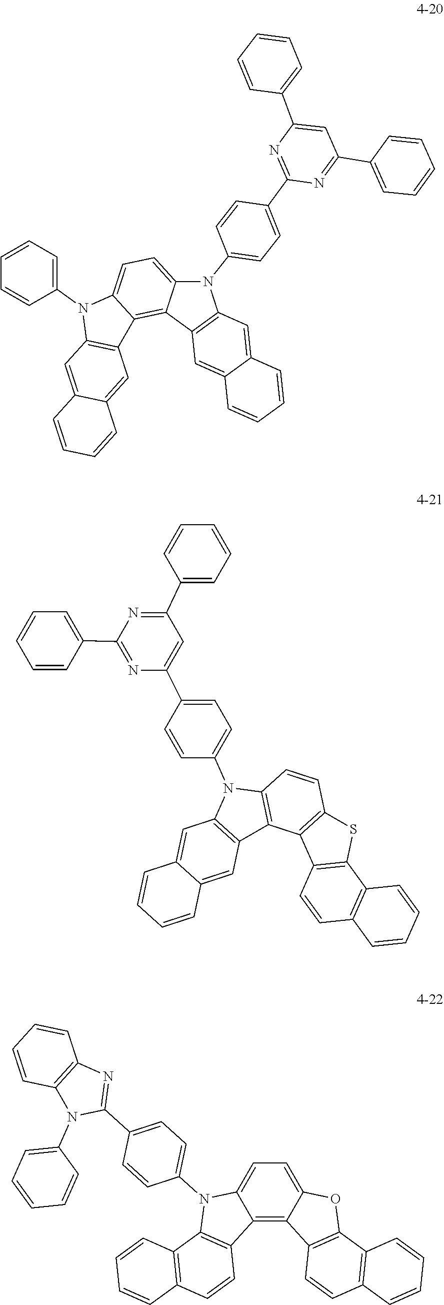 [SMILES: c1ccc(-c2cc(-c3ccccc3)nc(-c3ccc(-n4c5cc6ccccc6cc5c5c6c7cc8ccccc8cc7n(-c7ccccc7)c6ccc54)cc3)n2)cc1 ; c1ccc(-c2cc(-c3ccc(-n4c5cc6ccccc6cc5c5c6c(ccc54)sc4c5ccccc5ccc46)cc3)nc(-c3ccccc3)n2)cc1 ; c1ccc(-n2c(-c3ccc(-n4c5ccc6oc7c8ccccc8ccc7c6c5c5ccc6ccccc6c54)cc3)nc3ccccc32)cc1]

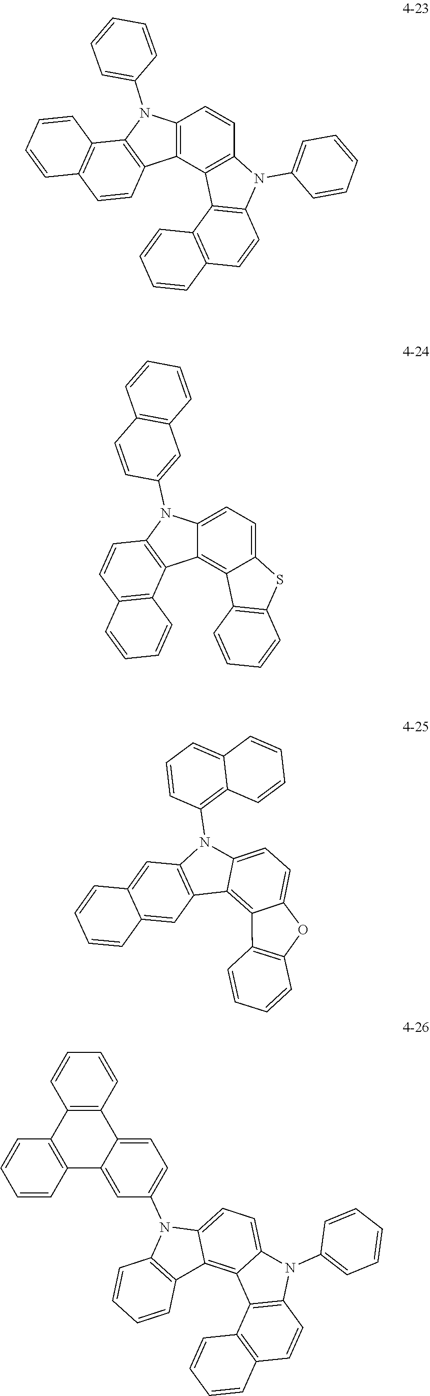 [SMILES: c1ccc(-n2c3ccc4ccccc4c3c3c4c5ccc6ccccc6c5n(-c5ccccc5)c4ccc32)cc1 ; c1ccc2cc(-n3c4ccc5ccccc5c4c4c5c(ccc43)sc3ccccc35)ccc2c1 ; c1ccc2cc3c(cc2c1)c1c2c(ccc1n3-c1cccc3ccccc13)oc1ccccc12 ; c1ccc(-n2c3ccc4ccccc4c3c3c4c5ccccc5n(-c5ccc6c7ccccc7c7ccccc7c6c5)c4ccc32)cc1]

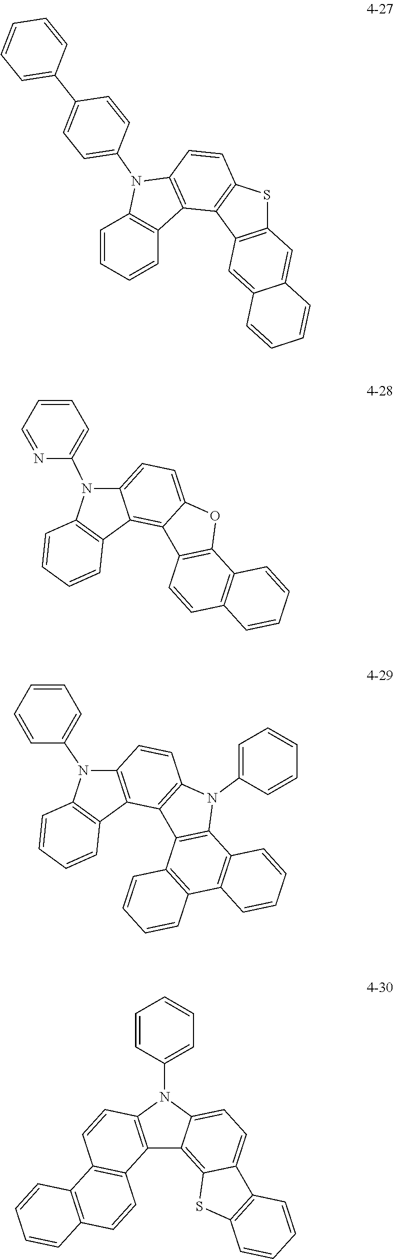 [SMILES: c1ccc(-c2ccc(-n3c4ccccc4c4c5c(ccc43)sc3cc4ccccc4cc35)cc2)cc1 ; c1ccc(-n2c3ccccc3c3c4c(ccc32)oc2c3ccccc3ccc24)nc1 ; c1ccc(-n2c3ccccc3c3c4c5c6ccccc6c6ccccc6c5n(-c5ccccc5)c4ccc32)cc1 ; c1ccc(-n2c3ccc4c5ccccc5ccc4c3c3c4sc5ccccc5c4ccc32)cc1]

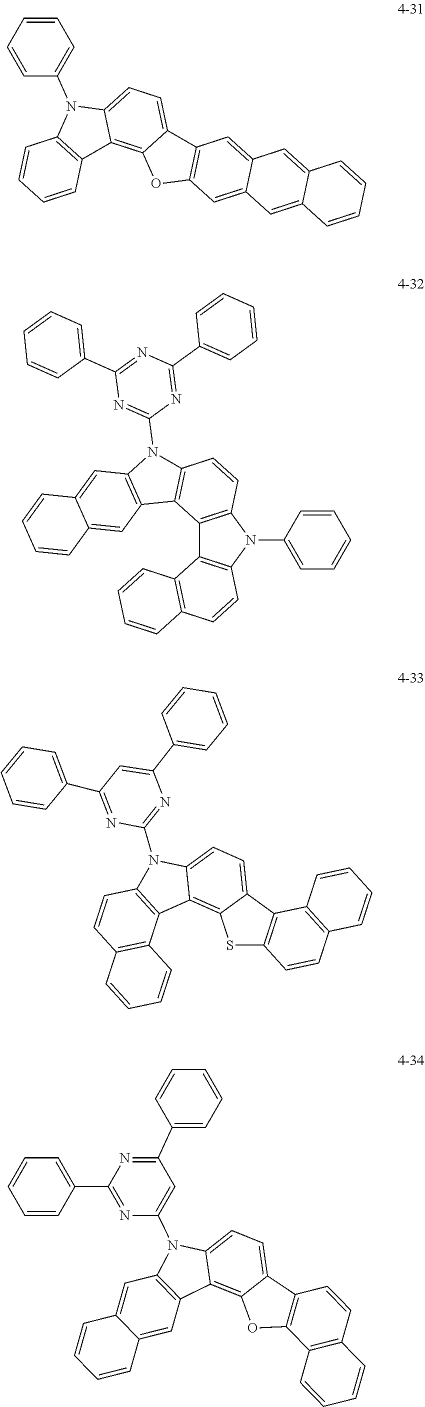 [SMILES: c1ccc(-n2c3ccccc3c3c4oc5cc6cc7ccccc7cc6cc5c4ccc32)cc1 ; c1ccc(-c2nc(-c3ccccc3)nc(-n3c4cc5ccccc5cc4c4c5c6c7ccccc7ccc6n(-c6ccccc6)c5ccc43)n2)cc1 ; c1ccc(-c2cc(-c3ccccc3)nc(-n3c4ccc5ccccc5c4c4c5sc6ccc7ccccc7c6c5ccc43)n2)cc1 ; c1ccc(-c2cc(-n3c4cc5ccccc5cc4c4c5oc6c7ccccc7ccc6c5ccc43)nc(-c3ccccc3)n2)cc1]

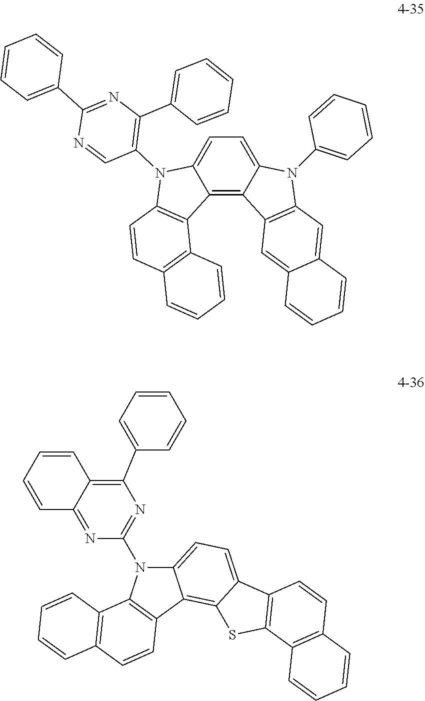 [SMILES: c1ccc(-c2ncc(-n3c4ccc5ccccc5c4c4c5c6cc7ccccc7cc6n(-c6ccccc6)c5ccc43)c(-c3ccccc3)n2)cc1 ; c1ccc(-c2nc(-n3c4ccc5c6ccc7ccccc7c6sc5c4c4ccc5ccccc5c43)nc3ccccc23)cc1]

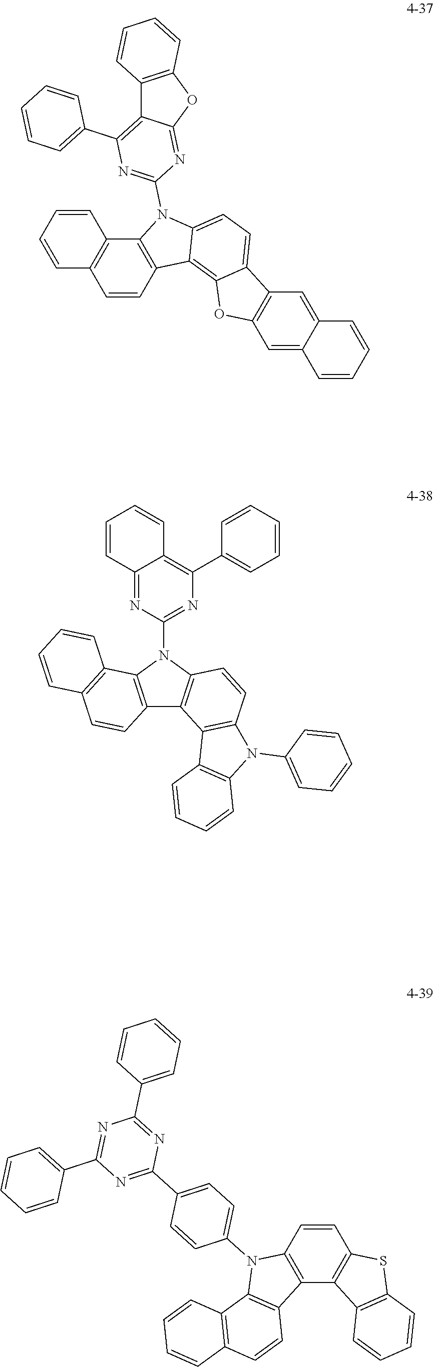 [SMILES: c1ccc(-c2nc(-n3c4ccc5c6cc7ccccc7cc6oc5c4c4ccc5ccccc5c43)nc3oc4ccccc4c23)cc1 ; c1ccc(-c2nc(-n3c4ccc5c(c6ccccc6n5-c5ccccc5)c4c4ccc5ccccc5c43)nc3ccccc23)cc1 ; c1ccc(-c2nc(-c3ccccc3)nc(-c3ccc(-n4c5ccc6sc7ccccc7c6c5c5ccc6ccccc6c54)cc3)n2)cc1]

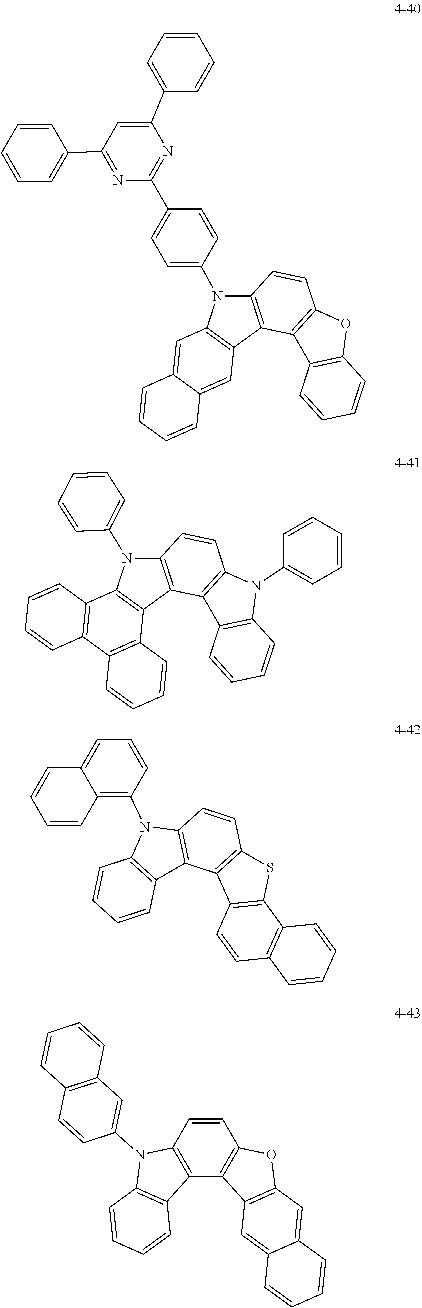

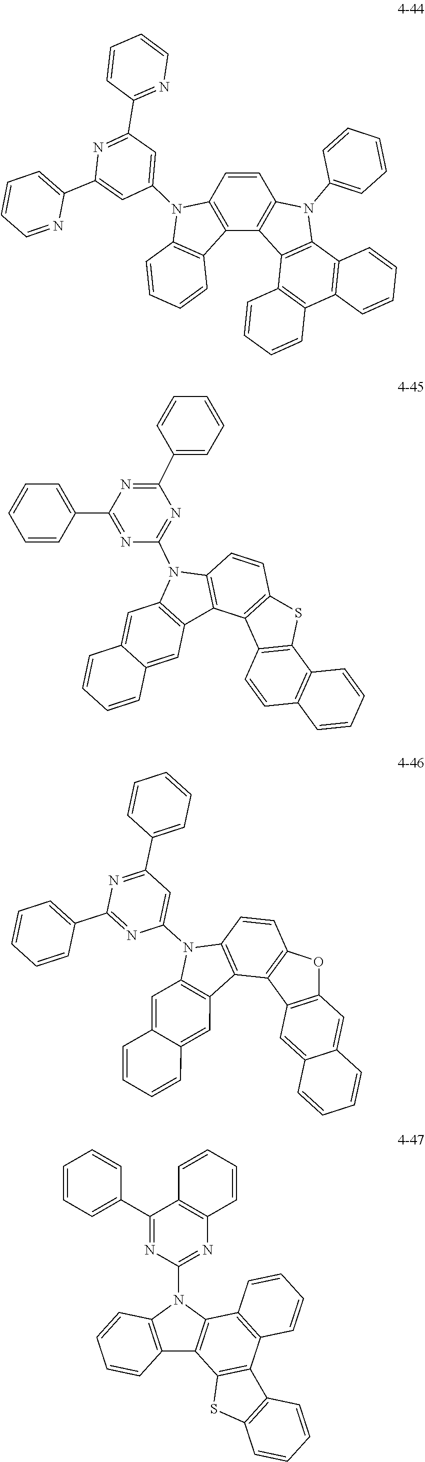 [SMILES: c1ccc(-n2c3ccc4c(c5ccccc5n4-c4cc(-c5ccccn5)nc(-c5ccccn5)c4)c3c3c4ccccc4c4ccccc4c32)cc1 ; c1ccc(-c2nc(-c3ccccc3)nc(-n3c4cc5ccccc5cc4c4c5c(ccc43)sc3c4ccccc4ccc35)n2)cc1 ; c1ccc(-c2cc(-n3c4cc5ccccc5cc4c4c5c(ccc43)oc3cc4ccccc4cc35)nc(-c3ccccc3)n2)cc1 ; c1ccc(-c2nc(-n3c4ccccc4c4c5sc6ccccc6c5c5ccccc5c43)nc3ccccc23)cc1]

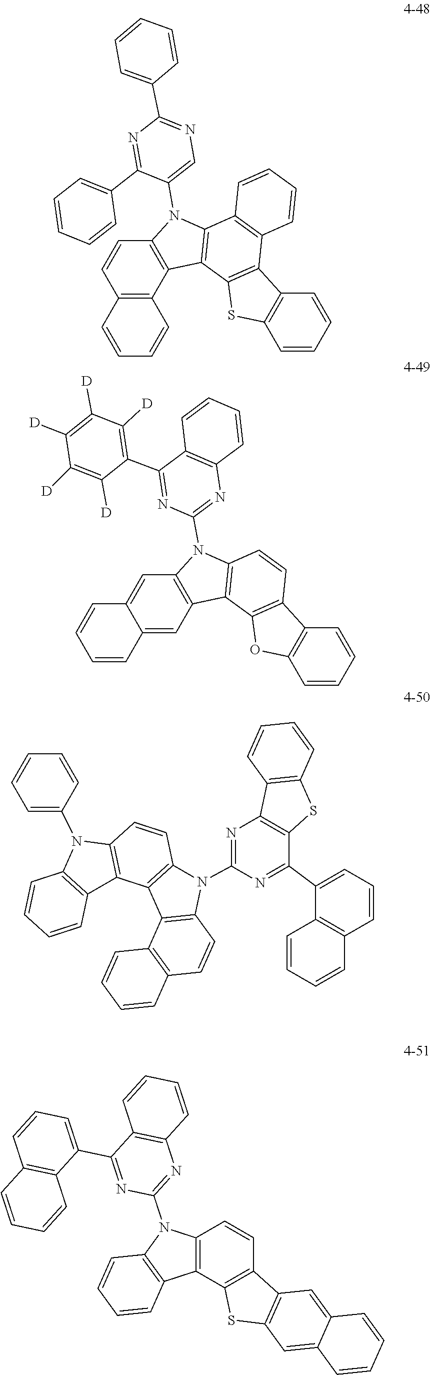

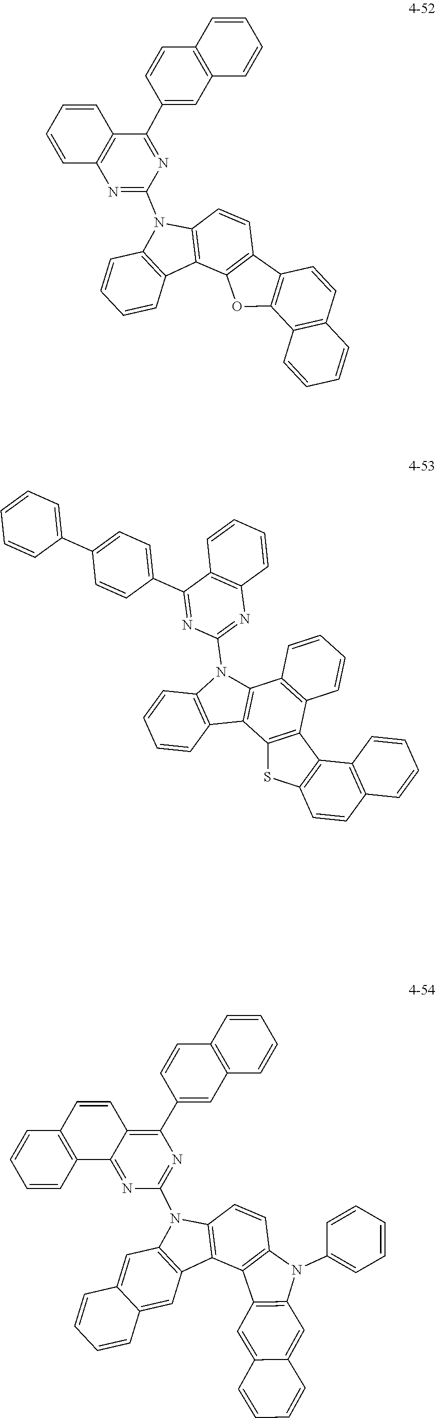 [SMILES: c1ccc2cc(-c3nc(-n4c5ccccc5c5c6oc7c8ccccc8ccc7c6ccc54)nc4ccccc34)ccc2c1 ; c1ccc(-c2ccc(-c3nc(-n4c5ccccc5c5c6sc7ccc8ccccc8c7c6c6ccccc6c54)nc4ccccc34)cc2)cc1 ; c1ccc(-n2c3cc4ccccc4cc3c3c4c5cc6ccccc6cc5n(-c5nc(-c6ccc7ccccc7c6)c6ccc7ccccc7c6n5)c4ccc32)cc1]

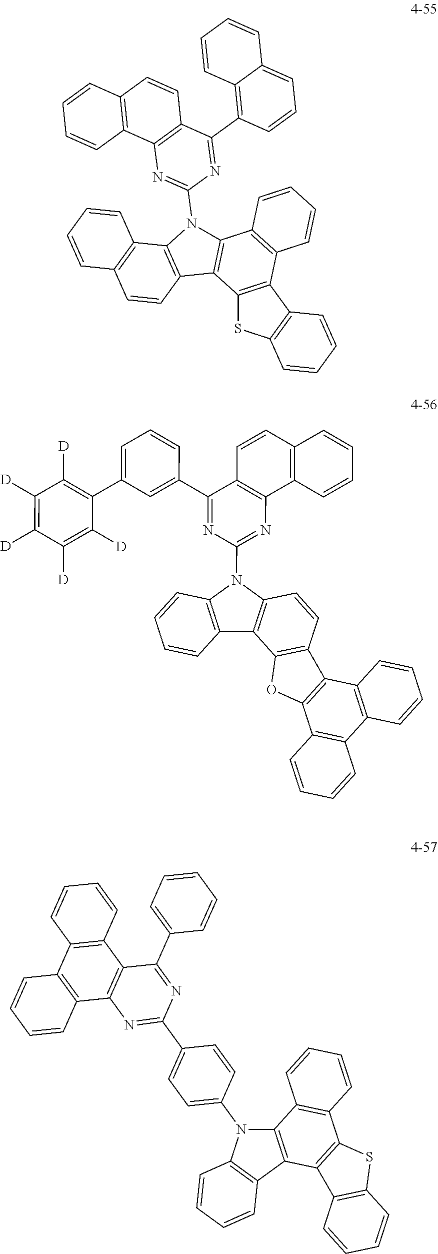 [SMILES: c1ccc2c(-c3nc(-n4c5c6ccccc6ccc5c5c6sc7ccccc7c6c6ccccc6c54)nc4c3ccc3ccccc34)cccc2c1 ; [2H]c1c([2H])c([2H])c(-c2cccc(-c3nc(-n4c5ccccc5c5c6oc7c8ccccc8c8ccccc8c7c6ccc54)nc4c3ccc3ccccc34)c2)c([2H])c1[2H] ; c1ccc(-c2nc(-c3ccc(-n4c5ccccc5c5c6c7ccccc7sc6c6ccccc6c54)cc3)nc3c4ccccc4c4ccccc4c23)cc1]

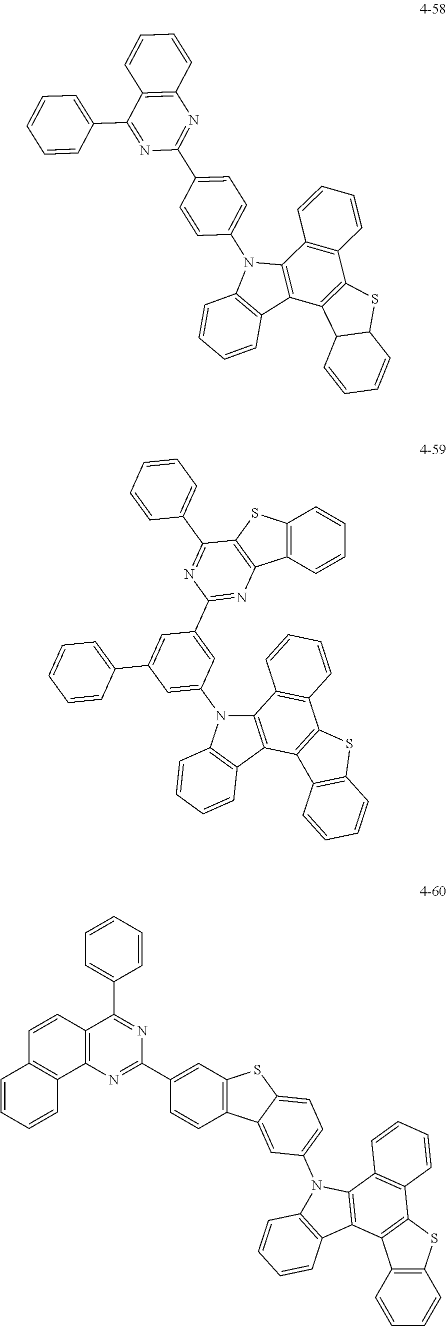 [SMILES: C1=CC2Sc3c(c4c5ccccc5n(-c5ccc(-c6nc(-c7ccccc7)c7ccccc7n6)cc5)c4c4ccccc34)C2C=C1 ; c1ccc(-c2cc(-c3nc(-c4ccccc4)c4sc5ccccc5c4n3)cc(-n3c4ccccc4c4c5c6ccccc6sc5c5ccccc5c43)c2)cc1 ; c1ccc(-c2nc(-c3ccc4c(c3)sc3ccc(-n5c6ccccc6c6c7c8ccccc8sc7c7ccccc7c65)cc34)nc3c2ccc2ccccc23)cc1]

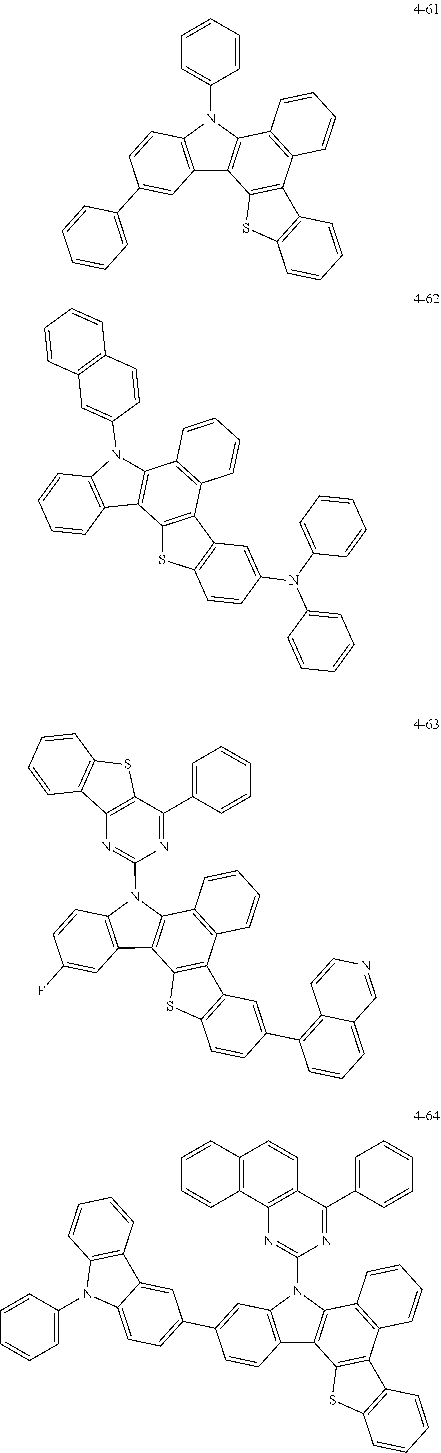

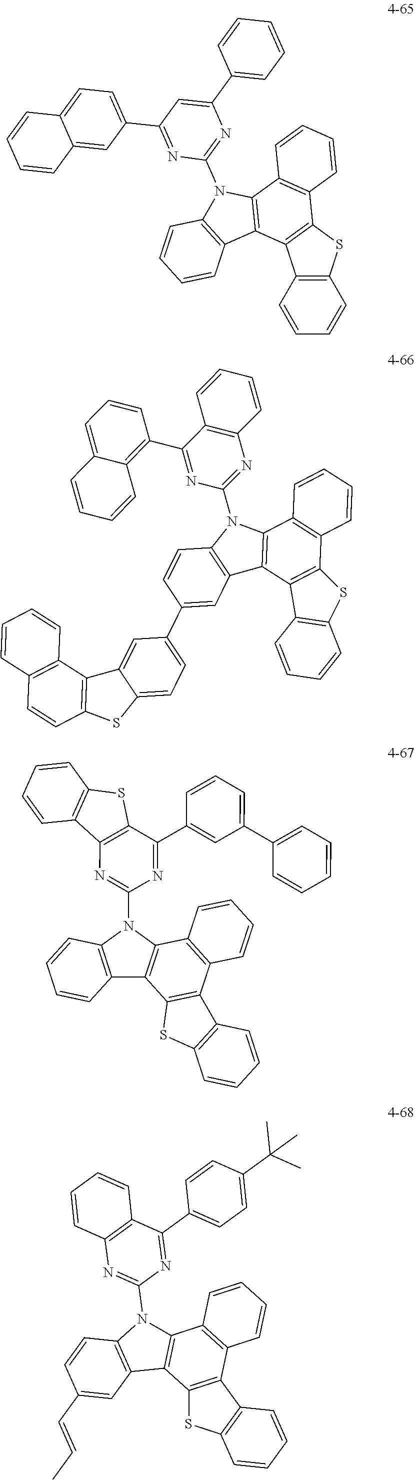

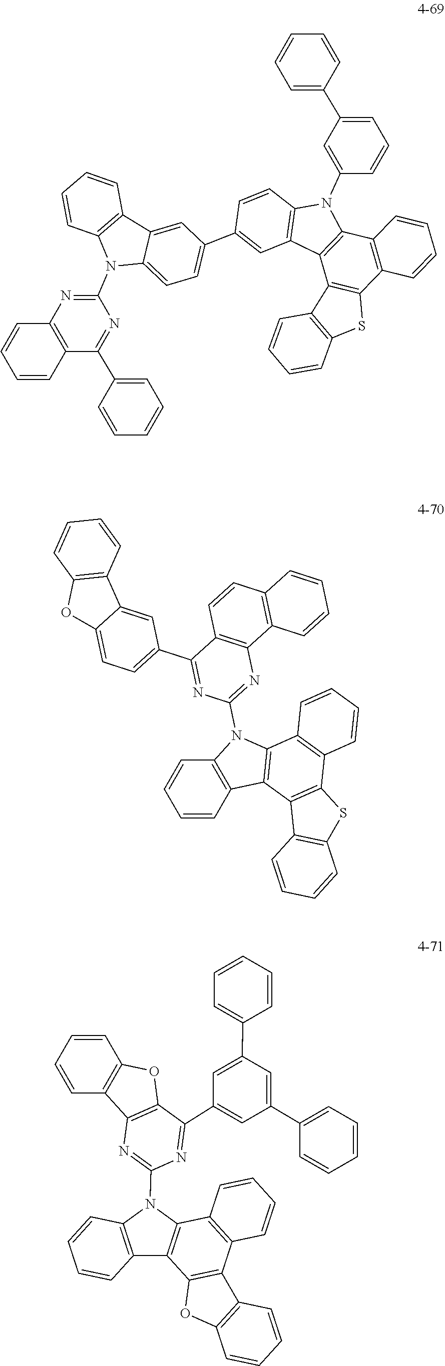 [SMILES: c1ccc(-c2cccc(-n3c4ccc(-c5ccc6c(c5)c5ccccc5n6-c5nc(-c6ccccc6)c6ccccc6n5)cc4c4c5c6ccccc6sc5c5ccccc5c43)c2)cc1 ; c1ccc2c(c1)ccc1c(-c3ccc4oc5ccccc5c4c3)nc(-n3c4ccccc4c4c5c6ccccc6sc5c5ccccc5c43)nc12 ; c1ccc(-c2cc(-c3ccccc3)cc(-c3nc(-n4c5ccccc5c5c6oc7ccccc7c6c6ccccc6c54)nc4c3oc3ccccc34)c2)cc1]

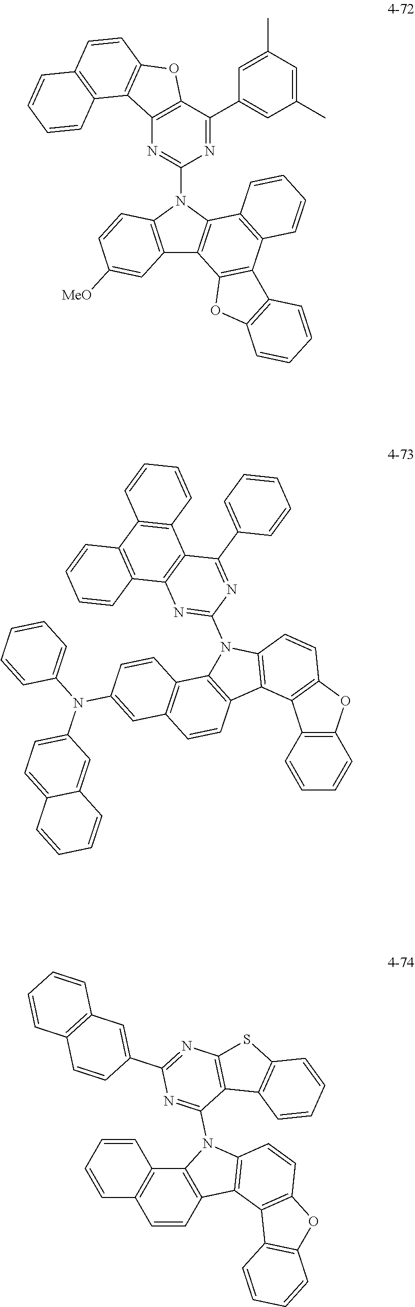 [SMILES: COc1ccc2c(c1)c1c3oc4ccccc4c3c3ccccc3c1n2-c1nc(-c2cc(C)cc(C)c2)c2oc3ccc4ccccc4c3c2n1 ; c1ccc(-c2nc(-n3c4ccc5oc6ccccc6c5c4c4ccc5cc(N(c6ccccc6)c6ccc7ccccc7c6)ccc5c43)nc3c4ccccc4c4ccccc4c23)cc1 ; c1ccc2cc(-c3nc(-n4c5ccc6oc7ccccc7c6c5c5ccc6ccccc6c54)c4c(n3)sc3ccccc34)ccc2c1]

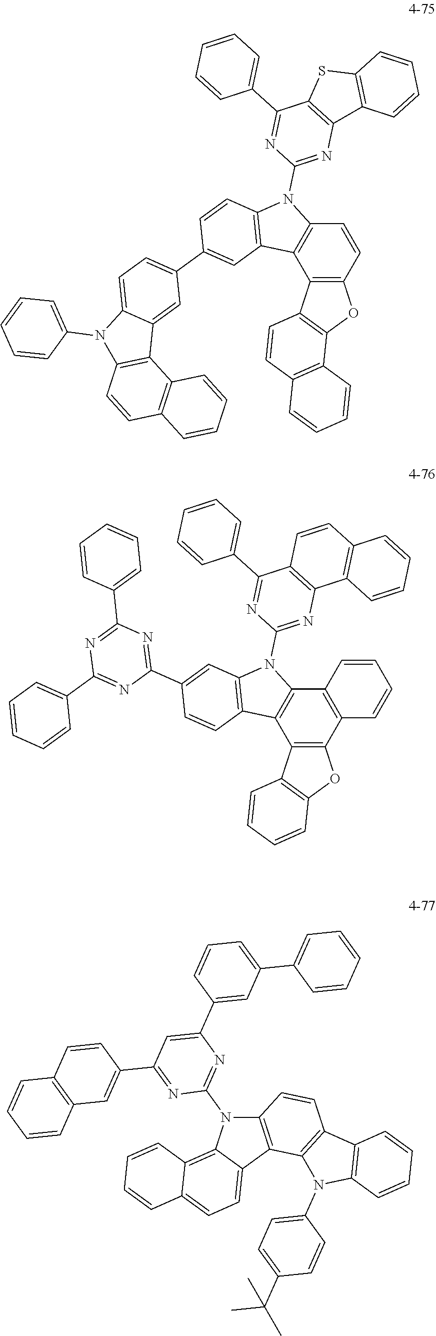 [SMILES: c1ccc(-c2nc(-n3c4ccc(-c5ccc6c(c5)c5c7ccccc7ccc5n6-c5ccccc5)cc4c4c5c(ccc43)oc3c4ccccc4ccc35)nc3c2sc2ccccc23)cc1 ; c1ccc(-c2nc(-c3ccccc3)nc(-c3ccc4c5c6c7ccccc7oc6c6ccccc6c5n(-c5nc(-c6ccccc6)c6ccc7ccccc7c6n5)c4c3)n2)cc1 ; CC(C)(C)c1ccc(-n2c3ccccc3c3ccc4c(c5ccc6ccccc6c5n4-c4nc(-c5cccc(-c6ccccc6)c5)cc(-c5ccc6ccccc6c5)n4)c32)cc1]

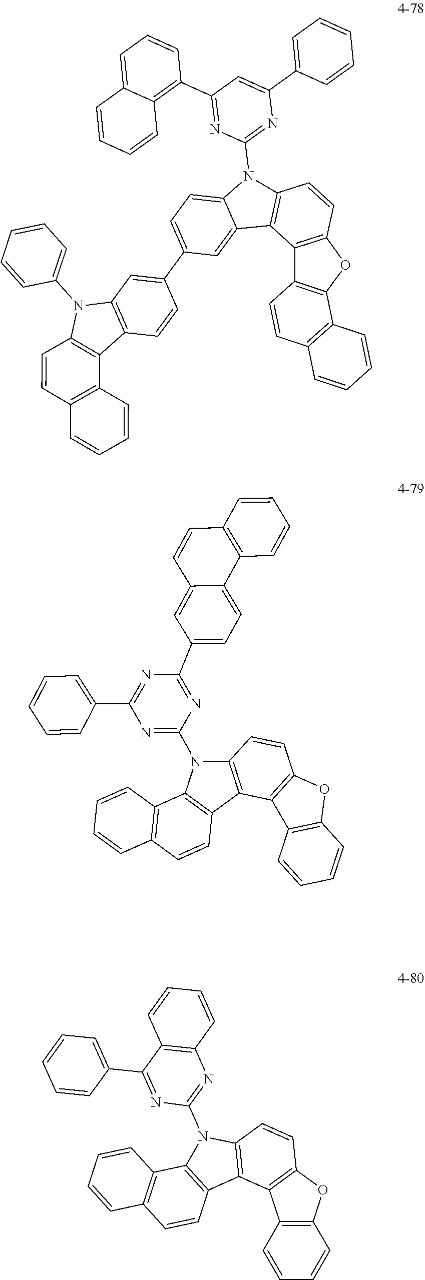 [SMILES: c1ccc(-c2cc(-c3cccc4ccccc34)nc(-n3c4ccc(-c5ccc6c7c8ccccc8ccc7n(-c7ccccc7)c6c5)cc4c4c5c(ccc43)oc3c4ccccc4ccc35)n2)cc1 ; c1ccc(-c2nc(-c3ccc4c(ccc5ccccc54)c3)nc(-n3c4ccc5oc6ccccc6c5c4c4ccc5ccccc5c43)n2)cc1 ; c1ccc(-c2nc(-n3c4ccc5oc6ccccc6c5c4c4ccc5ccccc5c43)nc3ccccc23)cc1]

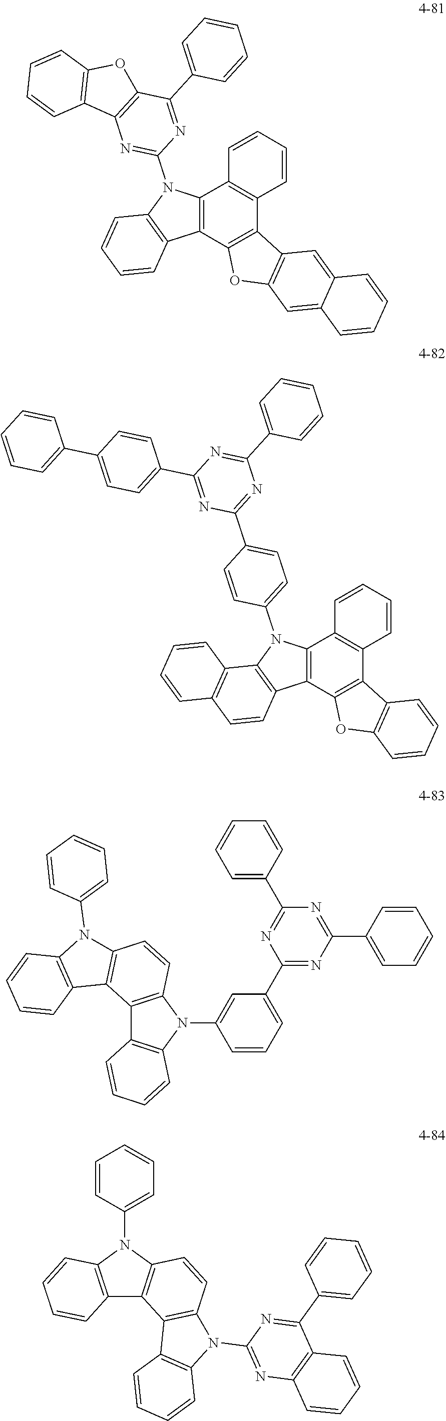

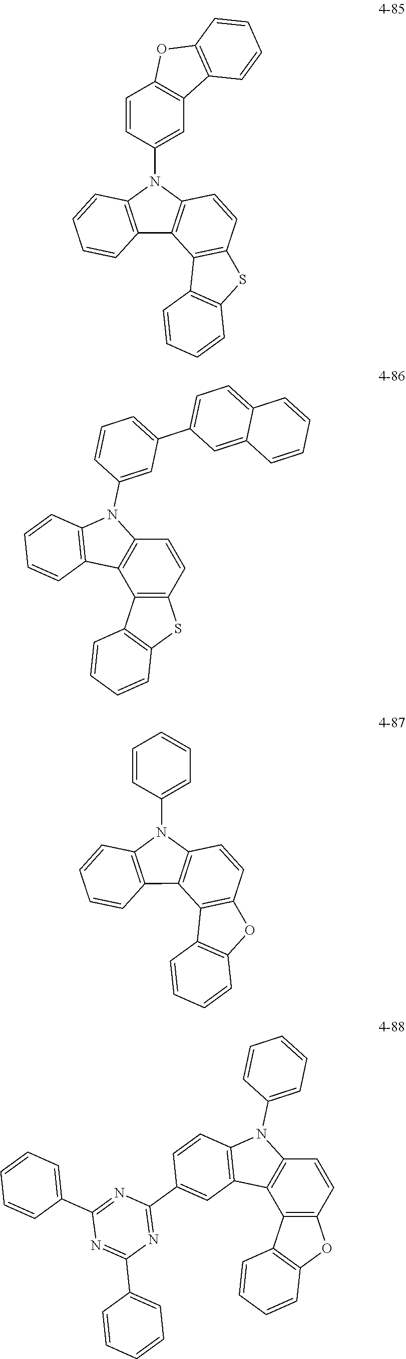

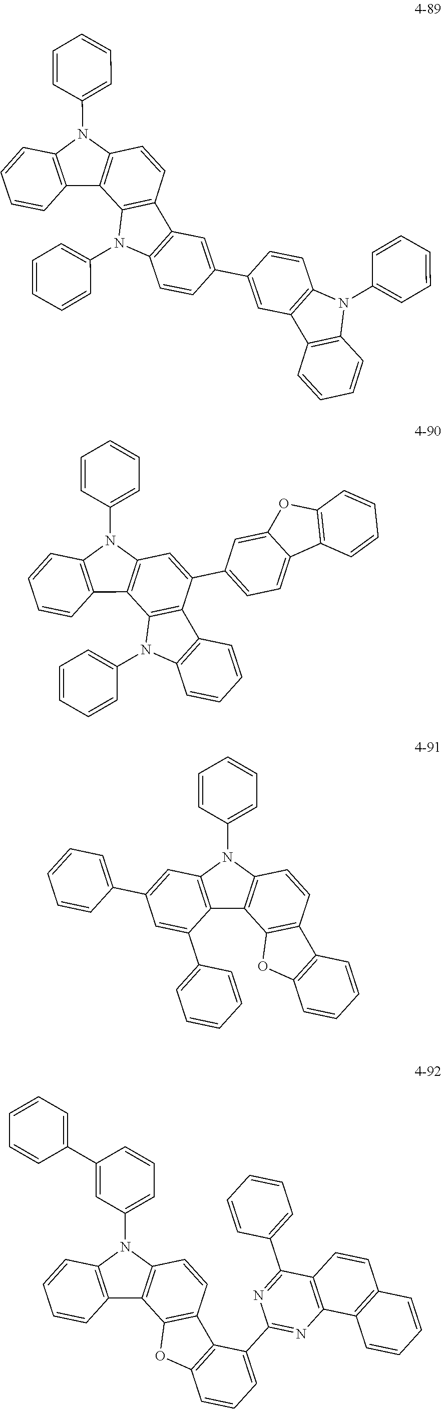 [SMILES: c1ccc(-n2c3ccccc3c3cc(-c4ccc5c(c4)c4ccc6c(c7ccccc7n6-c6ccccc6)c4n5-c4ccccc4)ccc32)cc1 ; c1ccc(-n2c3ccccc3c3c2cc(-c2ccc4c(c2)oc2ccccc24)c2c4ccccc4n(-c4ccccc4)c23)cc1 ; c1ccc(-c2cc(-c3ccccc3)c3c4c5oc6ccccc6c5ccc4n(-c4ccccc4)c3c2)cc1 ; c1ccc(-c2cccc(-n3c4ccccc4c4c5oc6cccc(-c7nc(-c8ccccc8)c8ccc9ccccc9c8n7)c6c5ccc43)c2)cc1]

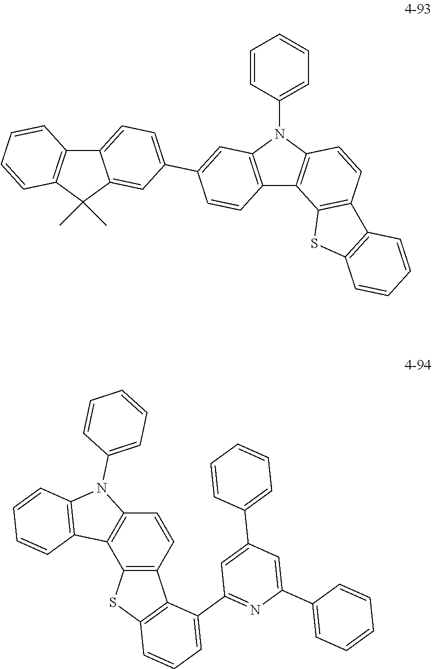 [SMILES: CC1(C)c2ccccc2-c2ccc(-c3ccc4c5c6sc7ccccc7c6ccc5n(-c5ccccc5)c4c3)cc21 ; c1ccc(-c2cc(-c3ccccc3)nc(-c3cccc4sc5c(ccc6c5c5ccccc5n6-c5ccccc5)c34)c2)cc1]

Also, the present invention provides a compound comprising at least one hole transport band layer between the anode and the emitting layer, wherein the hole transport band layer comprises a hole transport layer, an emitting auxiliary layer, or both, and the hole transport band layer comprises a compound represented by Formula 1.

It may further include a light efficiency enhancing layer formed on at least one surface opposite to the organic material layer of one surface of the anode and the cathode. Moreover, the organic material layer may comprise 2 or more stacks comprising a hole transport layer, an emitting layer, and an electron transport layer sequentially formed on the anode, and the organic material layer may further comprise a charge generating layer formed between 2 or more stacks.

In another aspect, the present invention provides an electronic device comprising a display device comprising the organic electronic element; and a control unit for driving the display device. In this case, the organic electronic element is at least one of an OLED, an organic solar cell, an organic photo conductor (OPC), an organic transistor (organic TFT), and an element for monochromic or white illumination.

Hereinafter, examples of the synthesis of the compound represented by Formula according to the present invention and the preparation of the organic electric device will be described in detail with reference to examples, but the present invention is not limited to the following examples.

### Synthesis Example 1

The compound (Final product 1) represented by Formula 1 according to the present invention may be prepared by reacting as shown in Reaction Scheme 1, but is not limited thereto.

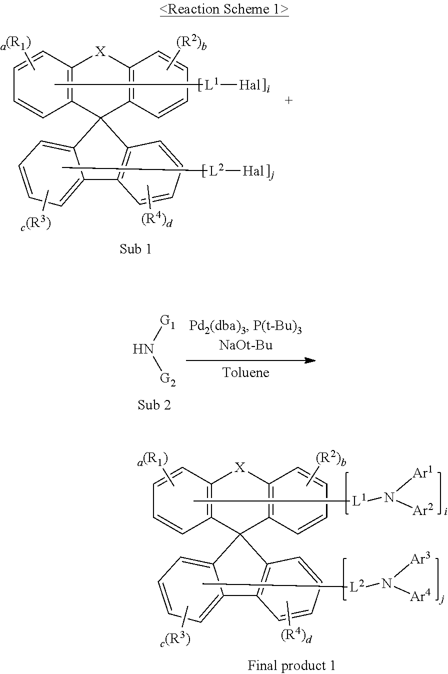

Wherein, Hal₁ and Hal₂ are Cl, Br or I, G₁ is Ar¹ or Ar³, G₂ is Ar² or Ar⁴.

### I. Synthesis Example of Sub 1

Sub 1 of Reaction Scheme 1 may be synthesized by the reaction route of Reaction Scheme 2, but is not limited thereto.

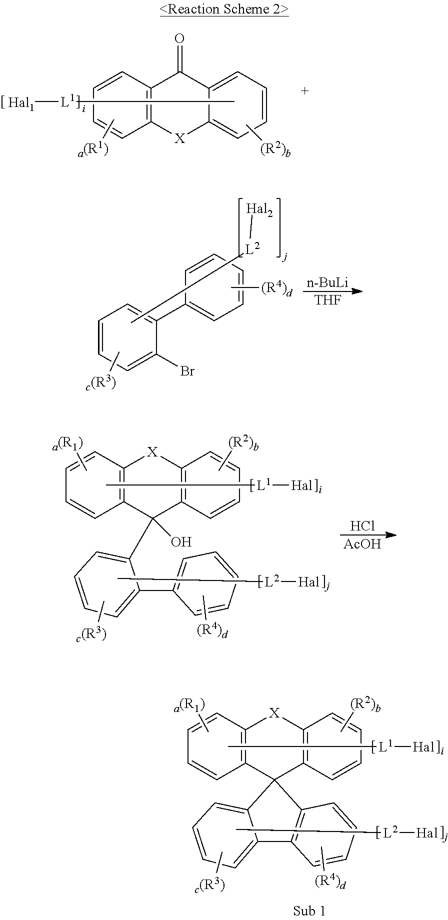

Synthesis examples of specific compounds belonging to Sub 1 are as follows.

### Synthesis Example of Sub 1-1

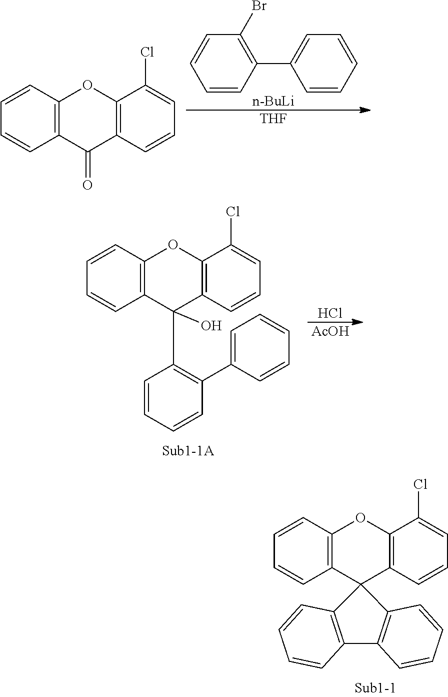

### (1) Synthesis of Sub 1-1A

4-chloro-9H-xanthen-9-one (20 g, 86.71 mmol) and 2-bromo-1,1′-biphenyl (21.22 g, 91.05 mmol) were dissolved in THE (600 ml), and then the temperature of the reaction mass was lowered to −78° C. After slowly adding n-BuLi (2.5 M in hexane) (6.11 g, 95.38 mmol), the reaction mixture was stirred at room temperature for 4 hours. When the reaction is completed, the reactant is quenched by putting it in H₂O, then water in the reactant is removed, filtered under reduced pressure, the product produced by concentrating the organic solvent was separated using column chromatography to obtain 29.7 g of the product. (Yield: 89%)

### (2) Synthesis of Sub 1-1

Sub 1-1A (20 g, 51.97 mmol), HCl (4 ml), Acetic acid (208 ml) were added and stirred at 80° C. for 1 hour. When the reaction was completed, after filtration under reduced pressure, the product produced by concentrating the organic solvent was separated using column chromatography to obtain 17.54 g of the product. (Yield: 92%)

### Synthesis Example of Sub 1-6

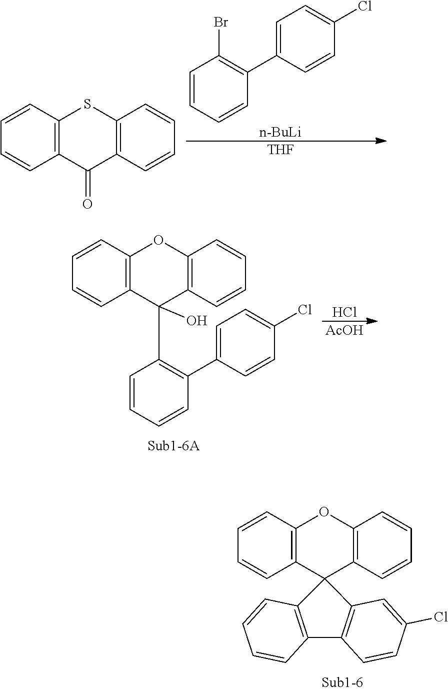

### (1) Synthesis of Sub 1-6A

4-chloro-9H-xanthen-9-one (20 g, 101.93 mmol) and 2-bromo-1,1′-biphenyl (28.64 g, 107.03 mmol), THE (680 ml), n-BuLi (2.5 M in hexane) (7.18 g, 112.12 mmol) were carried out in the same manner as in Synthesis method of Sub1-1A to obtain a product (33.3 g, 85%).

### (2) Synthesis of Sub 1-6

Sub 1-6A (20 g, 51.97 mmol), HCl (4 ml), Acetic acid (208 ml) were carried out in the same manner as in Synthesis method of Sub1-1 to obtain a product (16.78 g, 88%).

### Synthesis Example of Sub 1-46

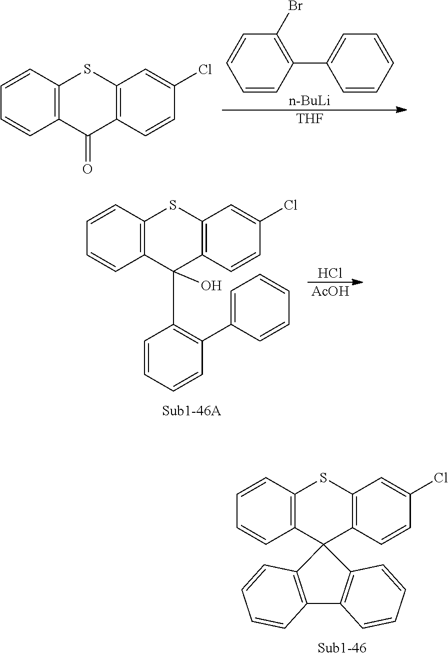

### (1) Synthesis of Sub 1-46A

3-chloro-9H-thioxanthen-9-one (20 g, 81.07 mmol) and 2-bromo-1,1′-biphenyl (19.84 g, 85.12 mmol), THE (600 ml), n-BuLi (2.5 M in hexane) (5.71 g, 89.17 mmol) were carried out in the same manner as in Synthesis method of Sub1-1A to obtain a product (25.7 g, 79%).

### (2) Synthesis of Sub 1-46

Sub 1-46A (20.8 g, 51.97 mmol), HCl (4 ml), Acetic acid (200 ml) were carried out in the same manner as in Synthesis method of Sub1-1 to obtain a product (15.47 g, 81%).

### Synthesis Example of Sub 1-55

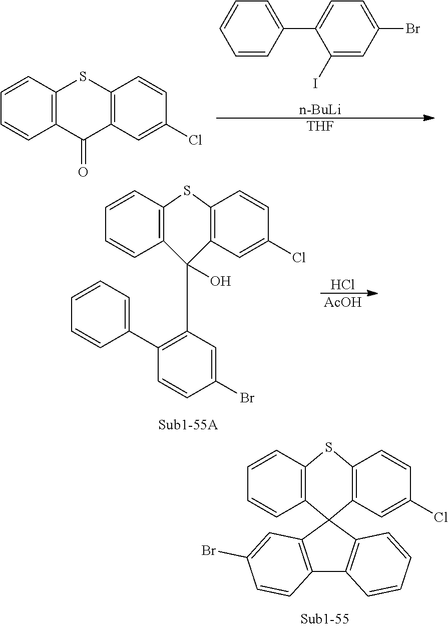

### (1) Synthesis of Sub 1-55A

2-chloro-9H-thioxanthen-9-one (20 g, 81.07 mmol) and 4-bromo-2-iodo-1,1′-biphenyl (30.56 g, 85.12 mmol), THE (600 ml), n-BuLi (2.5 M in hexane) (5.71 g, 89.17 mmol) were carried out in the same manner as in Synthesis method of Sub1-1A to obtain a product (33.06 g, 85%).

### (2) Synthesis of Sub 1-55

Sub 1-55A (20 g, 41.68 mmol), HCl (3.5 ml), Acetic acid (167 ml) were carried out in the same manner as in Synthesis method of Sub1-1 to obtain a product (16.75 g, 87%).

### Synthesis Example of Sub 1-72

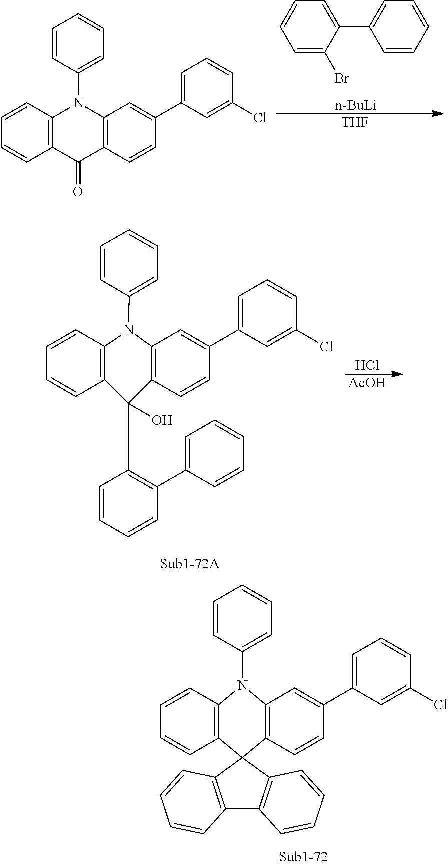

### (1) Synthesis of Sub 1-72A

3-(3-chlorophenyl)-10-phenylacridin-9(10H)-one (20 g, 52.38 mmol), 2-bromo-1,1′-biphenyl (12.82 g, 54.99 mmol), THE (500 ml), n-BuLi (2.5 M in hexane) (3.7 g, 57.61 mmol) were carried out in the same manner as in Synthesis method of Sub1-1A to obtain a product (25 g, 89%).

### (2) Synthesis of Sub 1-72

Sub 1-72A (20 g, 37.31 mmol), HCl (3 ml), Acetic acid (150 ml) were carried out in the same manner as in Synthesis method of Sub1-1 to obtain a product (17.59 g, 91%).

Furthermore, the compound belonging to Sub 1 may be a compound as follows, but is not limited thereto.

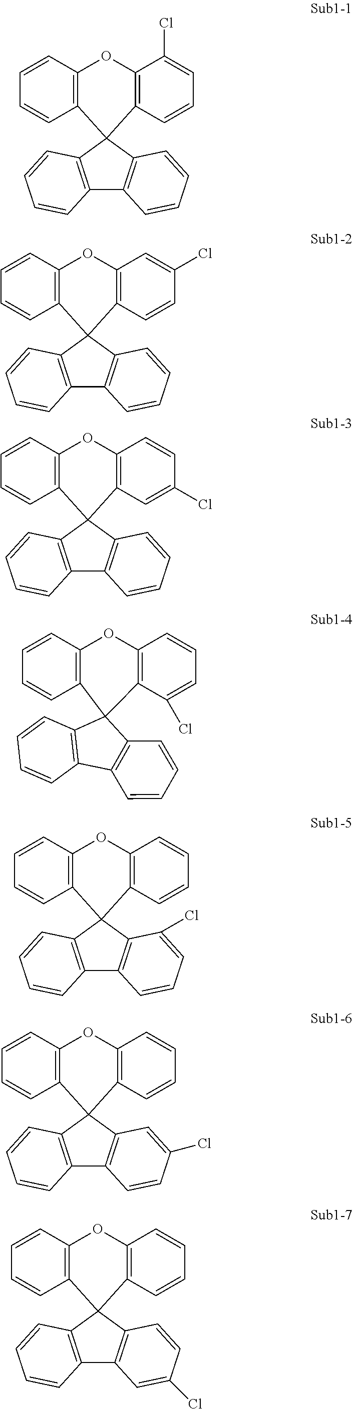

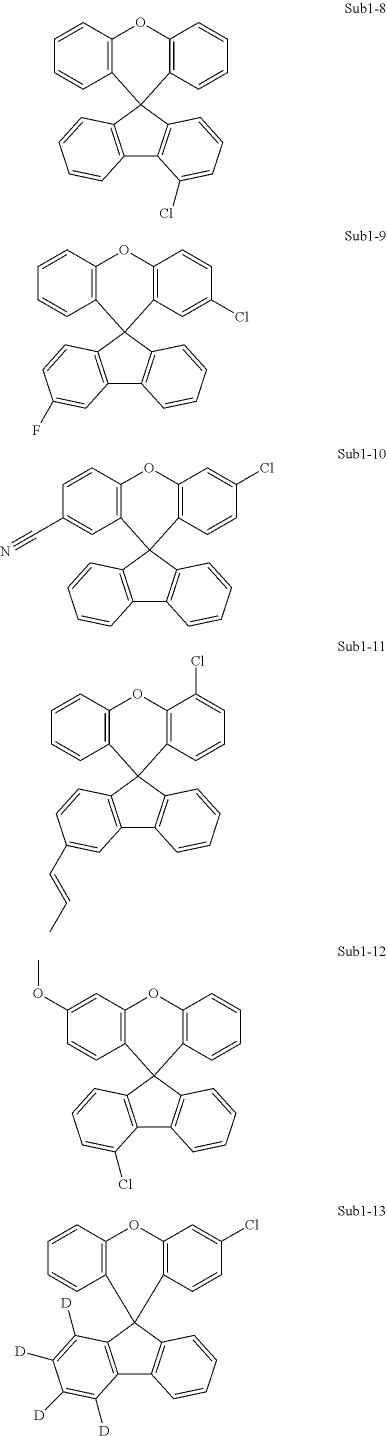

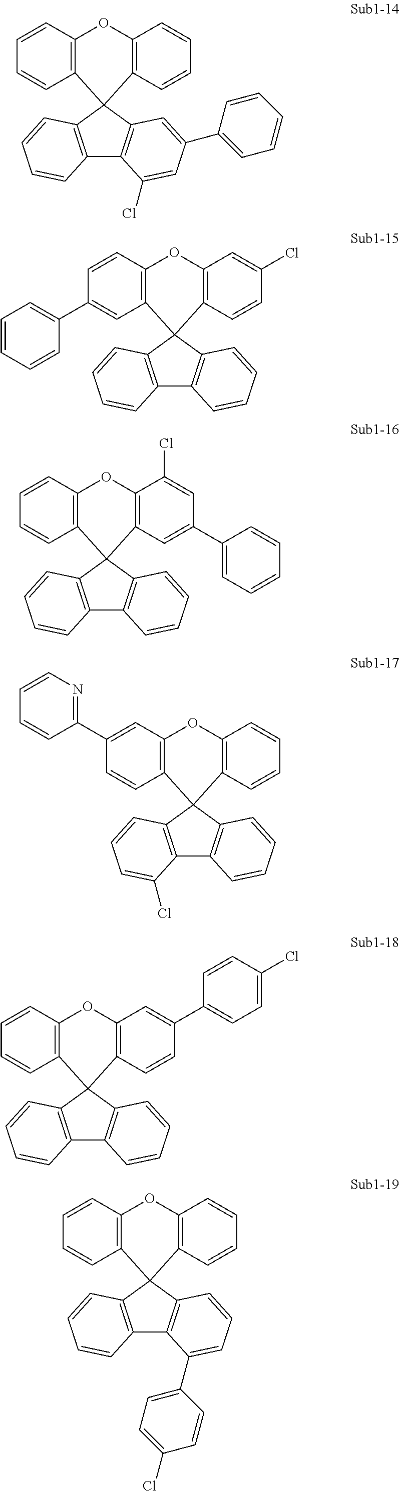

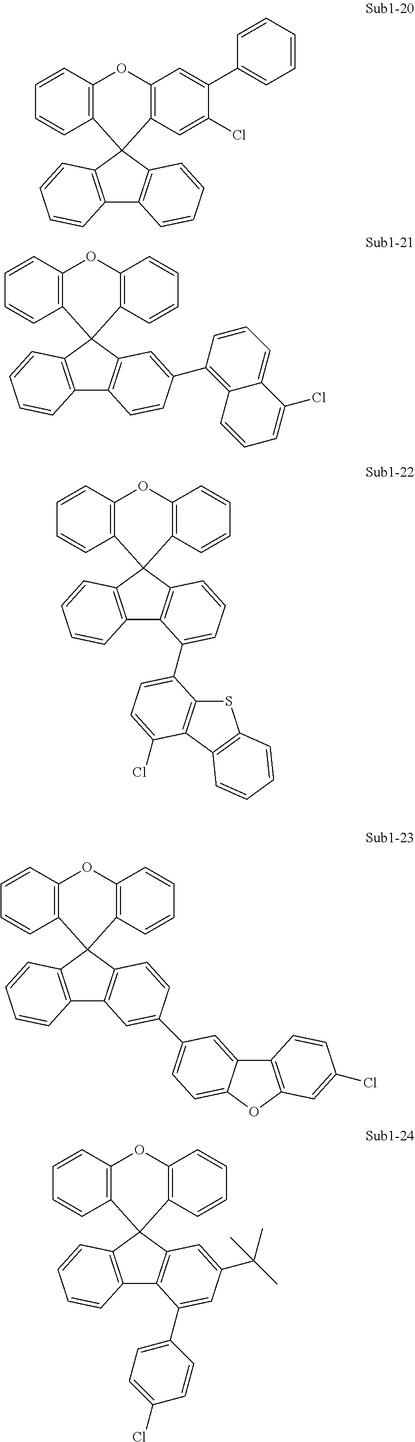

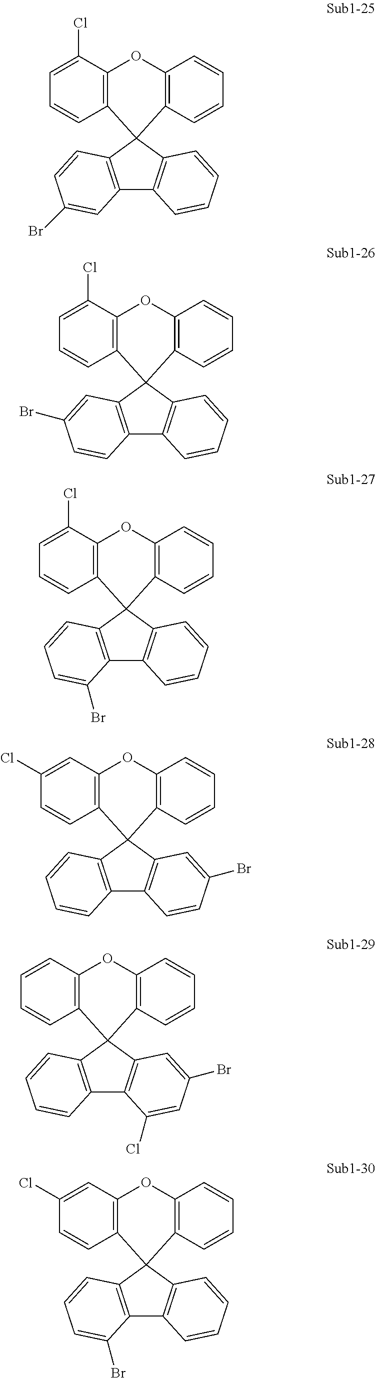

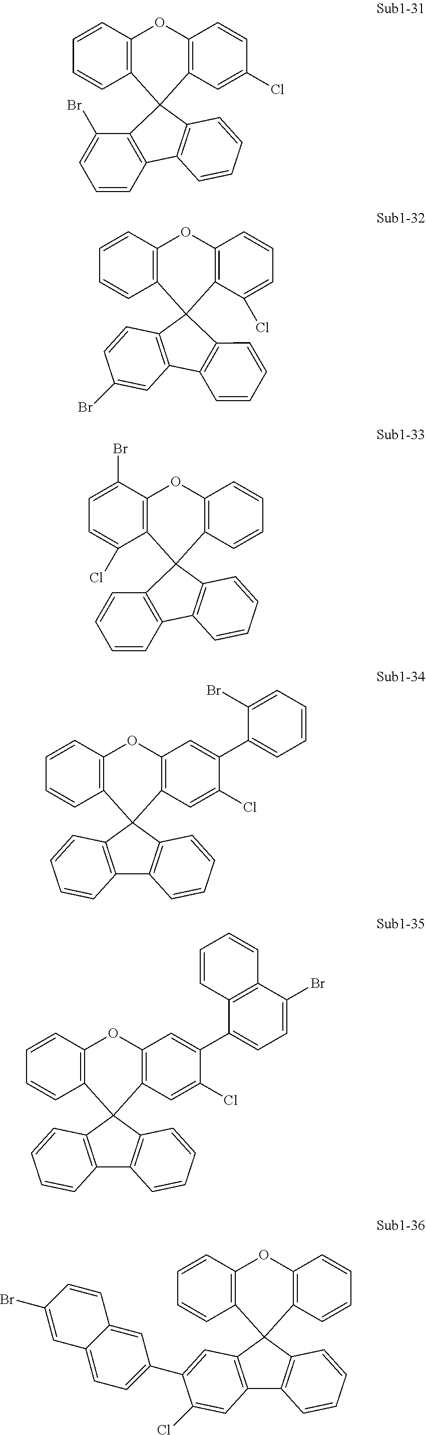

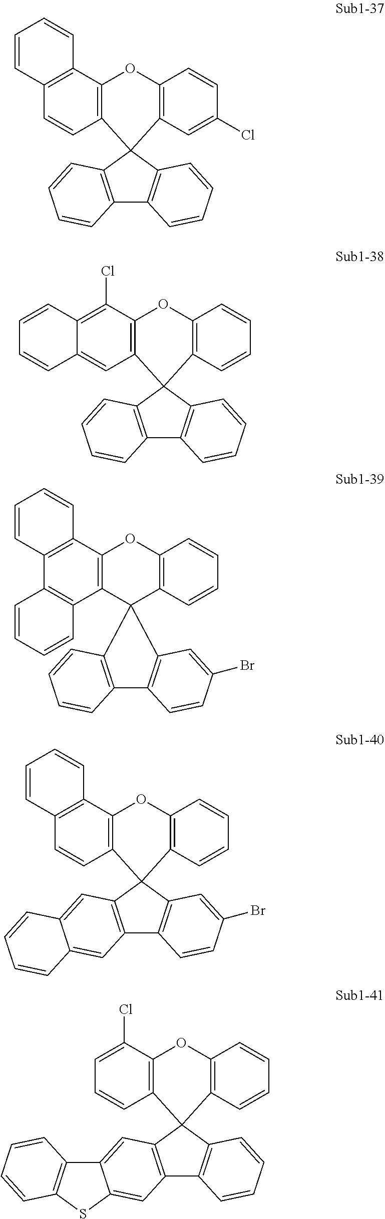

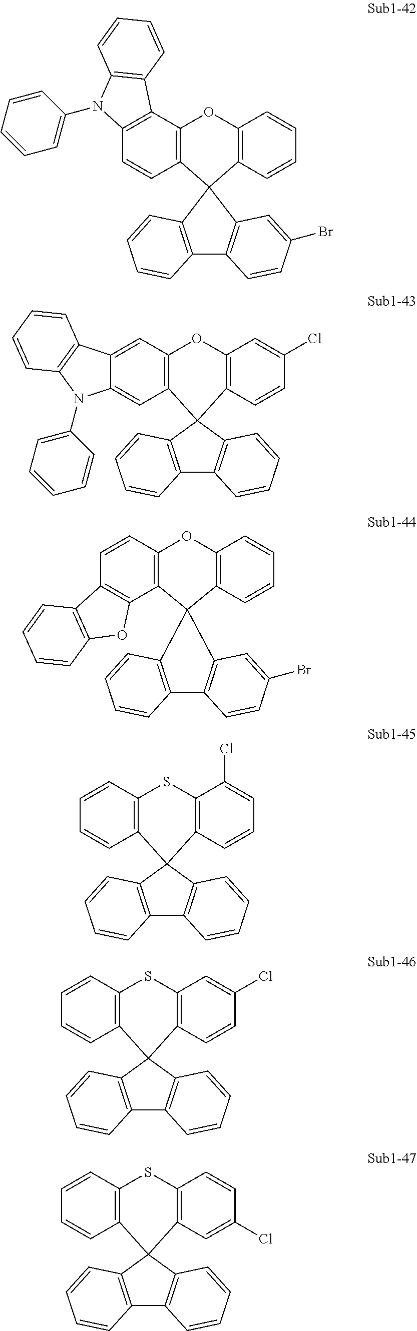

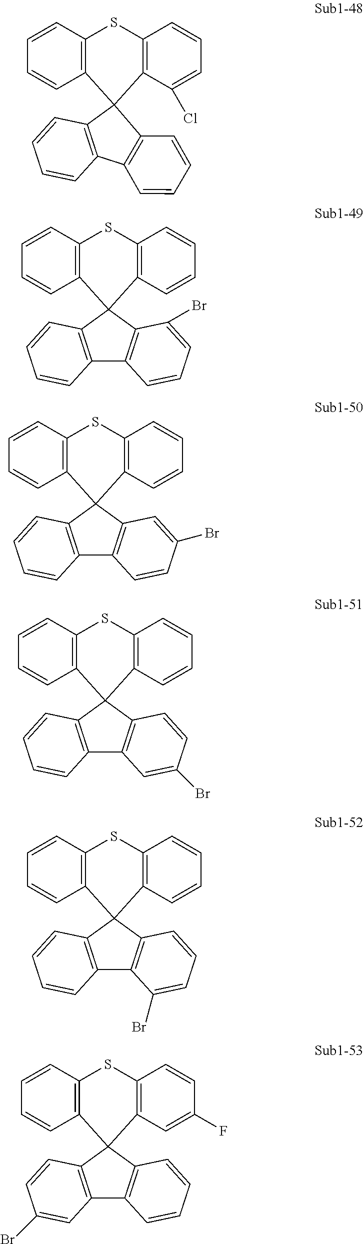

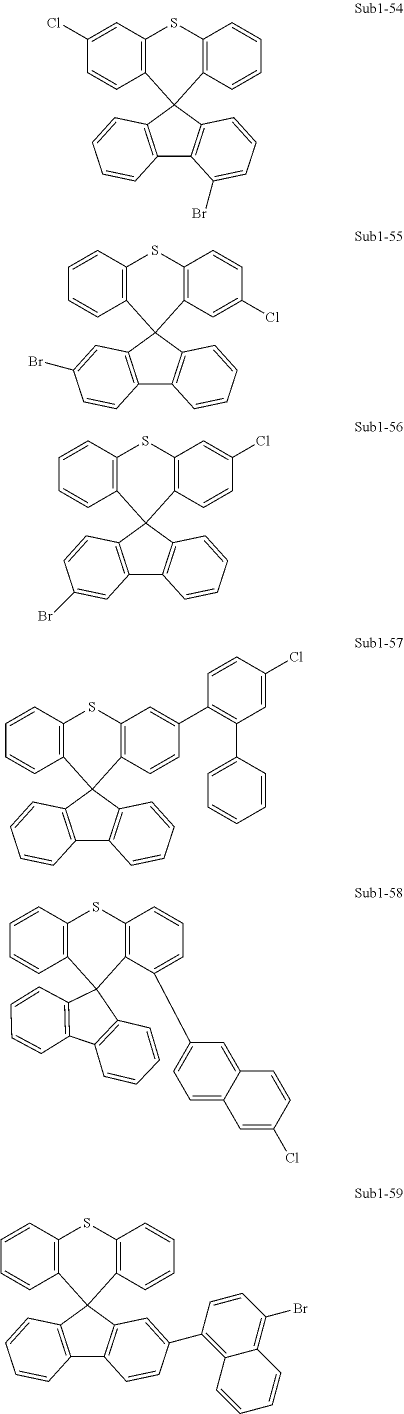

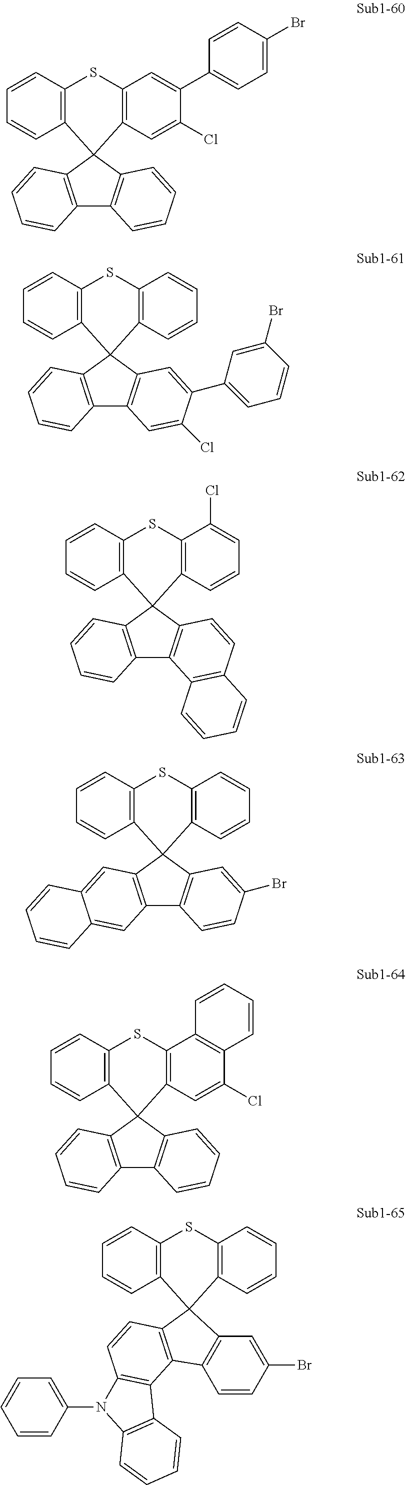

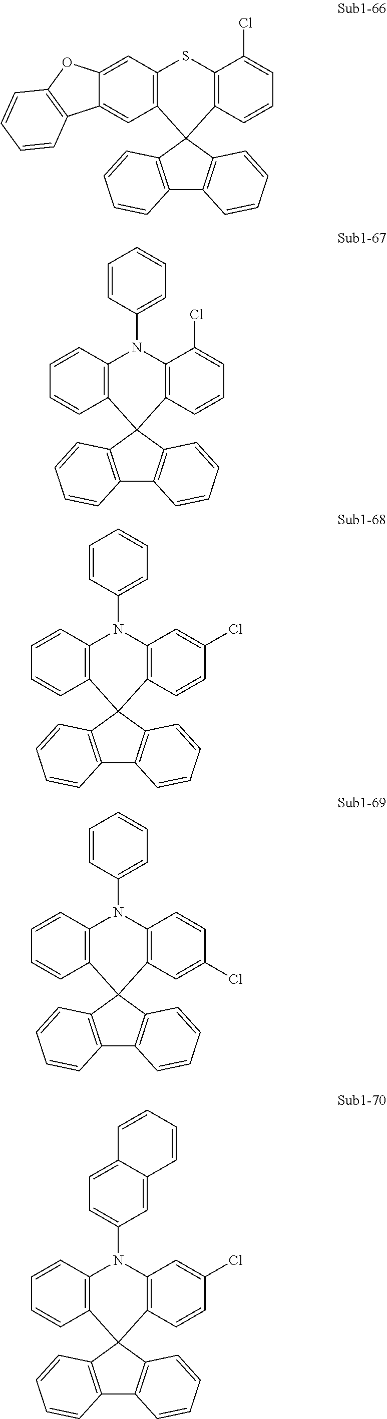

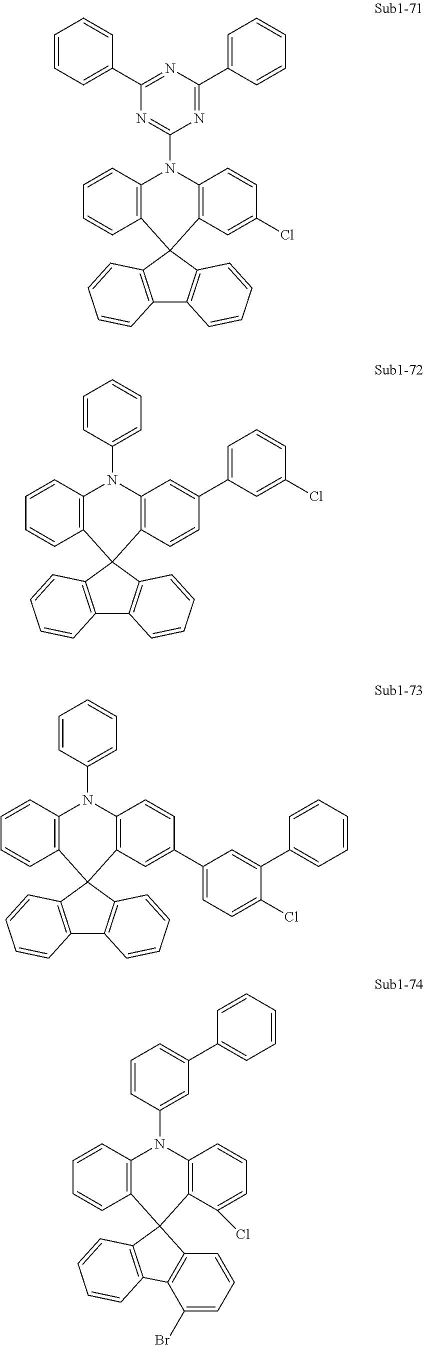 [SMILES: Clc1ccc2c(c1)C1(c3ccccc3-c3ccccc31)c1ccccc1N2c1nc(-c2ccccc2)nc(-c2ccccc2)n1 ; Clc1cccc(-c2ccc3c(c2)N(c2ccccc2)c2ccccc2C32c3ccccc3-c3ccccc32)c1 ; Clc1ccc(-c2ccc3c(c2)C2(c4ccccc4-c4ccccc42)c2ccccc2N3c2ccccc2)cc1-c1ccccc1 ; Clc1cccc2c1C1(c3ccccc3-c3c(Br)cccc31)c1ccccc1N2c1cccc(-c2ccccc2)c1]

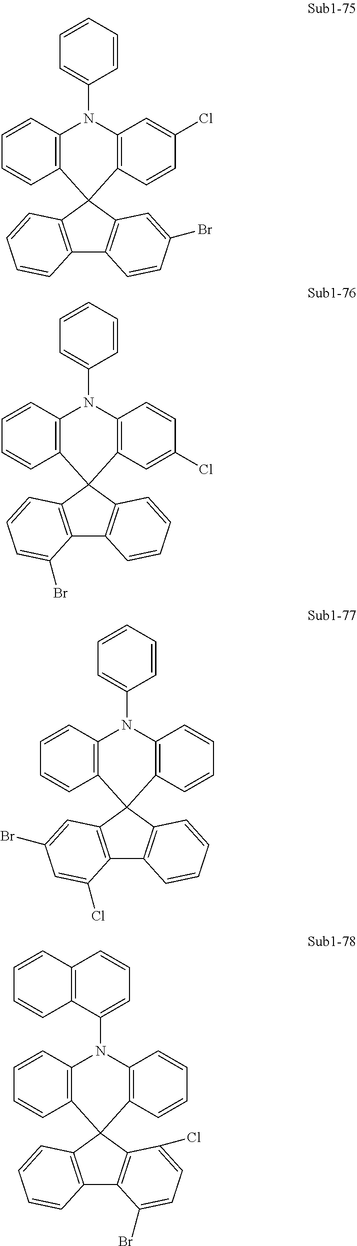

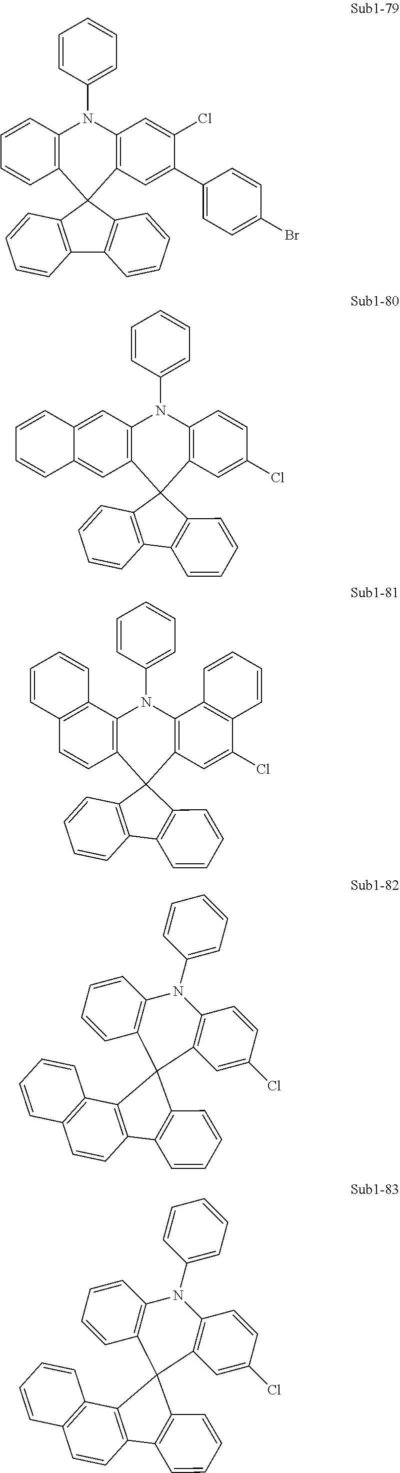

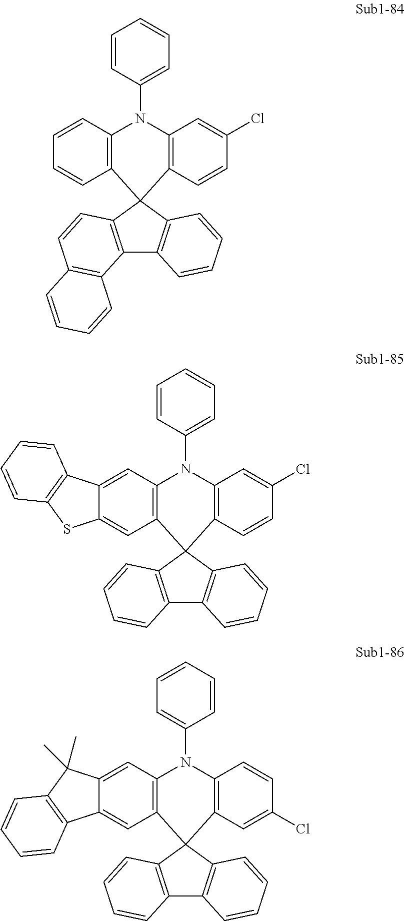 [SMILES: Clc1ccc2c(c1)N(c1ccccc1)c1ccccc1C21c2ccccc2-c2c1ccc1ccccc21 ; Clc1ccc2c(c1)N(c1ccccc1)c1cc3c(cc1C21c2ccccc2-c2ccccc21)sc1ccccc13 ; CC1(C)c2ccccc2-c2cc3c(cc21)N(c1ccccc1)c1ccc(Cl)cc1C31c2ccccc2-c2ccccc21]

Table 1 shows FD-MS (Field Desorption-Mass Spectrometry) values of compounds belonging to Sub 1.

TABLE 1

| com- | |
|---|---|
| pound | FD-MS |
| Sub 1-1 | m/z = 366.08(C₂₅H₁₅ClO = 366.84) |
| Sub 1-2 | m/z = 366.08(C₂₅H₁₅ClO = 366.84) |
| Sub 1-3 | m/z = 366.08(C₂₅H₁₅ClO = 366.84) |
| Sub 1-4 | m/z = 366.08(C₂₅H₁₅ClO = 366.84) |
| Sub 1-5 | m/z = 366.08(C₂₅H₁₅ClO = 366.84) |
| Sub 1-6 | m/z = 366.08(C₂₅H₁₅ClO = 366.84) |
| Sub 1-7 | m/z = 366.08(C₂₅H₁₅ClO = 366.84) |
| Sub 1-8 | m/z = 366.08(C₂₅H₁₅ClO = 366.84) |
| Sub 1-9 | m/z = 384.07(C₂₅H₁₄ClFO = 384.83) |
| Sub 1- | m/z = 391.08(C₂₆H₁₄ClNO = 391.85) |
| 10 | |
| Sub 1- | m/z = 406.11(C₂₈H₁₉ClO = 406.91) |
| 11 | |
| Sub 1- | m/z = 396.09(C₂₆H₁₇ClO₂ = 396.87) |
| 12 | |
| Sub 1- | m/z = 370.11(C₂₅H₁₁D₄ClO = 370.87) |
| 13 | |
| Sub 1- | m/z = 442.11(C₃₁H₁₉ClO = 442.94) |
| 14 | |
| Sub 1- | m/z = 442.11(C₃₁H₁₉ClO = 442.94) |
| 15 | |
| Sub 1- | m/z = 442.11(C₃₁H₁₉ClO = 442.94) |
| 16 | |
| Sub 1- | m/z = 443.11(C₃₀H₁₈ClNO = 443.93) |
| 17 | |
| Sub 1- | m/z = 442.11(C₃₁H₁₉ClO = 442.94) |
| 18 | |
| Sub 1- | m/z = 442.11(C₃₁H₁₉ClO = 442.94) |
| 19 | |
| Sub 1- | m/z = 442.11(C₃₁H₁₉ClO = 442.94) |
| 20 | |
| Sub 1- | m/z = 492.13(C₃₅H₂₁ClO = 493) |
| 21 | |
| Sub 1- | m/z = 548.1(C₃₇H₂₁ClOS = 549.08) |
| 22 | |
| Sub 1- | m/z = 532.12(C₃₇H₂₁ClO₂ = 533.02) |
| 23 | |
| Sub 1- | m/z = 498.18(C₃₅H₂₇ClO = 499.05) |
| 24 | |
| Sub 1- | m/z = 443.99(C₂₅H₁₄BrClO = 445.74) |
| 25 | |
| Sub 1- | m/z = 443.99(C₂₅H₁₄BrClO = 445.74) |
| 26 | |
| Sub 1- | m/z = 443.99(C₂₅H₁₄BrClO = 445.74) |
| 27 | |
| Sub 1- | m/z = 443.99(C₂₅H₁₄BrClO = 445.74) |
| 28 | |
| Sub 1- | m/z = 443.99(C₂₅H₁₄BrClO = 445.74) |
| 29 | |
| Sub 1- | m/z = 443.99(C₂₅H₁₄BrClO = 445.74) |
| 30 | |
| Sub 1- | m/z = 443.99(C₂₅H₁₄BrClO = 445.74) |
| 31 | |
| Sub 1- | m/z = 443.99(C₂₅H₁₄BrClO = 445.74) |
| 32 | |
| Sub 1- | m/z = 443.99(C₂₅H₁₄BrClO = 445.74) |
| 33 | |
| Sub 1- | m/z = 520.02(C₃₁H₁₈BrClO = 521.84) |
| 34 | |
| Sub 1- | m/z = 570.04(C₃₅H₂₀BrClO = 571.9) |
| 35 | |
| Sub 1- | m/z = 570.04(C₃₅H₂₀BrClO = 571.9) |
| 36 | |
| Sub 1- | m/z = 416.1(C₂₉H₁₇ClO = 416.9) |
| 37 | |
| Sub 1- | m/z = 416.1(C₂₉H₁₇ClO = 416.9) |
| 38 | |
| Sub 1- | m/z = 510.06(C₃₃H₁₉BrO = 511.42) |
| 39 | |
| Sub 1- | m/z = 510.06(C₃₃H₁₉BrO = 511.42) |
| 40 | |
| Sub 1- | m/z = 472.07(C₃₁H₁₇ClOS = 472.99) |
| 41 | |
| Sub 1- | m/z = 575.09(C₃₇H₂₂BrNO = 576.49) |
| 42 | |
| Sub 1- | m/z = 531.14(C₃₇H₂₂ClNO = 532.04) |
| 43 | |
| Sub 1- | m/z = 500.04(C₃₁H₁₇BrO₂ = 501.38) |
| 44 | |
| Sub 1- | m/z = 382.06(C₂₅H₁₅ClS = 382.91) |
| 45 | |
| Sub 1- | m/z = 382.06(C₂₅H₁₅ClS = 382.91) |
| 46 | |
| Sub 1- | m/z = 382.06(C₂₅H₁₅ClS = 382.91) |
| 47 | |
| Sub 1- | m/z = 382.06(C₂₅H₁₅ClS = 382.91) |
| 48 | |
| Sub 1- | m/z = 426.01(C₂₅H₁₅BrS = 427.36) |
| 49 | |
| Sub 1- | m/z = 426.01(C₂₅H₁₅BrS = 427.36) |
| 50 | |
| Sub 1- | m/z = 426.01(C₂₅H₁₅BrS = 427.36) |
| 51 | |
| Sub 1- | m/z = 426.01(C₂₅H₁₅BrS = 427.36) |
| 52 | |
| Sub 1- | m/z = 444(C₂₅H₁₄BrFS = 445.35) |
| 53 | |
| Sub 1- | m/z = 459.97(C₂₅H₁₄BrClS = 461.8) |
| 54 | |
| Sub 1- | m/z = 459.97(C₂₅H₁₄BrClS = 461.8) |
| 55 | |
| Sub 1- | m/z = 459.97(C₂₅H₁₄BrClS = 461.8) |
| 56 | |
| Sub 1- | m/z = 534.12(C₃₇H₂₃ClS = 535.1) |
| 57 | |
| Sub 1- | m/z = 508.11(C₃₅H₂₁ClS = 509.06) |
| 58 | |
| Sub 1- | m/z = 552.05(C₃₅H₂₁BrS = 553.52) |
| 59 | |
| Sub 1- | m/z = 536(C₃₁H₁₈BrClS = 537.9) |
| 60 | |
| Sub 1- | m/z = 536(C₃₁H₁₈BrClS = 537.9) |
| 61 | |
| Sub 1- | m/z = 432.07(C₂₉H₁₇ClS = 432.97) |
| 62 | |
| Sub 1- | m/z = 476.02(C₂₉H₁₇BrS = 477.42) |
| 63 | |
| Sub 1- | m/z = 432.07(C₂₉H₁₇ClS = 432.97) |
| 64 | |
| Sub 1- | m/z = 591.07(C₃₇H₂₂BrNS = 592.55) |
| 65 | |
| Sub 1- | m/z = 472.07(C₃₁H₁₇ClOS = 472.99) |
| 66 | |
| Sub 1- | m/z = 441.13(C₃₁H₂₀ClN = 441.96) |
| 67 | |
| Sub 1- | m/z = 441.13(C₃₁H₂₀ClN = 441.96) |
| 68 | |
| Sub 1- | m/z = 441.13(C₃₁H₂₀ClN = 441.96) |
| 69 | |
| Sub 1- | m/z = 491.14(C₃₅H₂₂ClN = 492.02) |
| 70 | |
| Sub 1- | m/z = 596.18(C₄₀H₂₅ClN₄ = 597.12) |
| 71 | |
| Sub 1- | m/z = 517.16(C₃₇H₂₄ClN = 518.06) |
| 72 | |
| Sub 1- | m/z = 593.19(C₄₃H₂₈ClN = 594.15) |
| 73 | |
| Sub 1- | m/z = 595.07(C₃₇H₂₃BrClN = 596.95) |
| 74 | |
| Sub 1- | m/z = 519.04(C₃₁H₁₉BrClN = 520.85) |
| 75 | |
| Sub 1- | m/z = 519.04(C₃₁H₁₉BrClN = 520.85) |
| 76 | |
| Sub 1- | m/z = 519.04(C₃₁H₁₉BrClN = 520.85) |
| 77 | |
| Sub 1- | m/z = 569.05(C₃₅H₂₁BrClN = 570.91) |
| 78 | |
| Sub 1- | m/z = 595.07(C₃₇H₂₃BrClN = 596.95) |
| 79 | |
| Sub 1- | m/z = 491.14(C₃₅H₂₂ClN = 492.02) |
| 80 | |
| Sub 1- | m/z = 541.16(C₃₉H₂₄ClN = 542.08) |
| 81 | |
| Sub 1- | m/z = 491.14(C₃₅H₂₂ClN = 492.02) |
| 82 | |
| Sub 1- | m/z = 491.14(C₃₅H₂₂ClN = 492.02) |
| 83 | |
| Sub 1- | m/z = 491.14(C₃₅H₂₂ClN = 492.02) |
| 84 | |
| Sub 1- | m/z = 547.12(C₃₇H₂₂ClNS = 548.1) |
| 85 | |
| Sub 1- | m/z = 557.19(C₄₀H₂₈ClN = 558.12) |
| 86 | |

### II. Synthesis Example of Sub 2

Sub 2 of Reaction Scheme 1 may be synthesized by the reaction route of Reaction Scheme 3, but is not limited thereto.

### Reaction Scheme 3

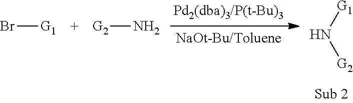

Wherein, G₁ is Ar¹ or Ar³, G₂ is Ar² or Ar⁴.

### Synthesis Example of Sub 2-1

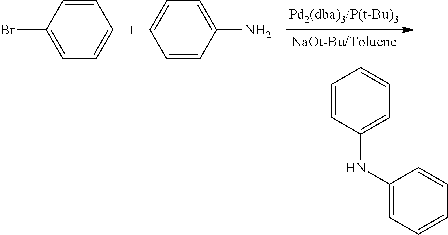

After adding bromobenzene (37.1 g, 236.2 mmol) to a round-bottom flask and dissolving it with toluene (2,200 mL), aniline (20 g, 214.8 mmol), Pd₂(dba)₃ (9.83 g, 10.7 mmol), P(t-Bu)₃ (4.34 g, 21.5 mmol), NaOt-Bu (62 g, 644.3 mmol) were added sequentially and stirred at 100° C. When the reaction was completed, the mixture was extracted with ether and water, the organic layer was dried over MgSO₄, concentrated, and the resulting compound was recrystallized using silicagel column to obtain 28 g (yield 77%) of Sub 2-1.

### Synthesis Example of Sub 2-37

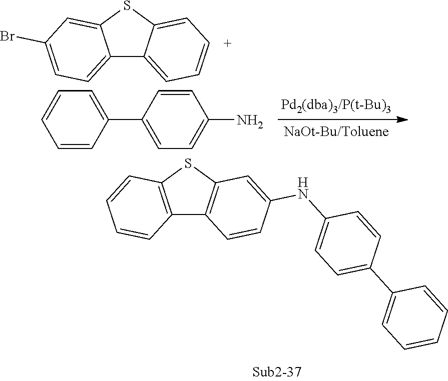

3-bromodibenzo[b,d]thiophene (42.8 g, 162.5 mmol), toluene (1,550 mL), [1,1′-biphenyl]-4-amine (25 g, 147.7 mmol), Pd₂(dba)₃ (6.76 g, 7.4 mmol), P(t-Bu)₃ (3 g, 14.8 mmol), NaOt-Bu (42.6 g, 443.2 mmol) were carried out in the same manner as in Synthesis method of Sub 2-1 to obtain Sub2-37 (37.9 g, 73%).

The compound belonging to Sub 2 may be a compound as follows, but is not limited thereto.

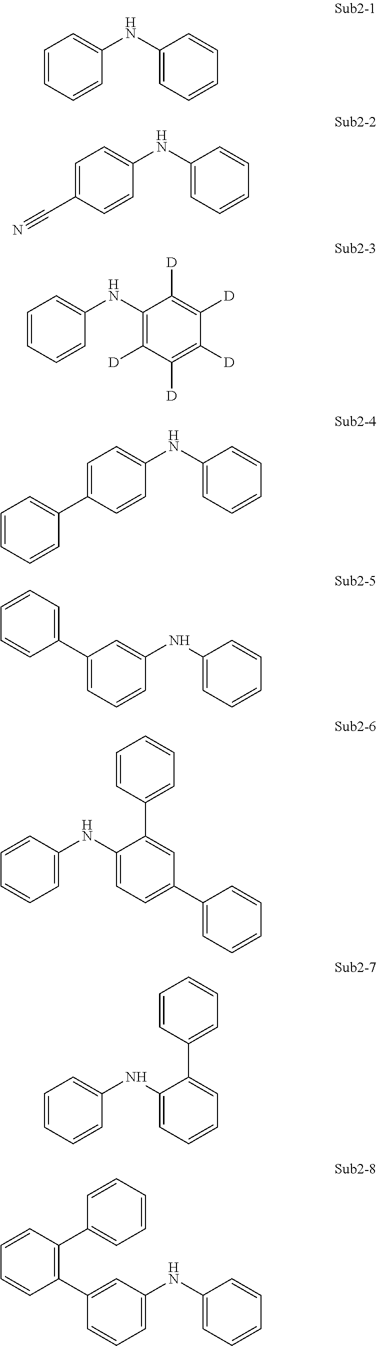

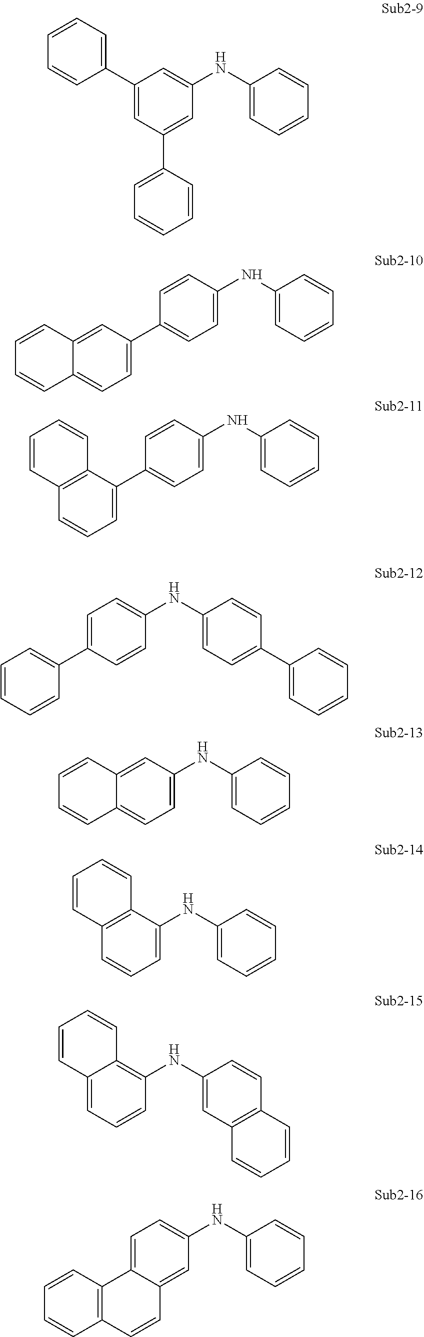

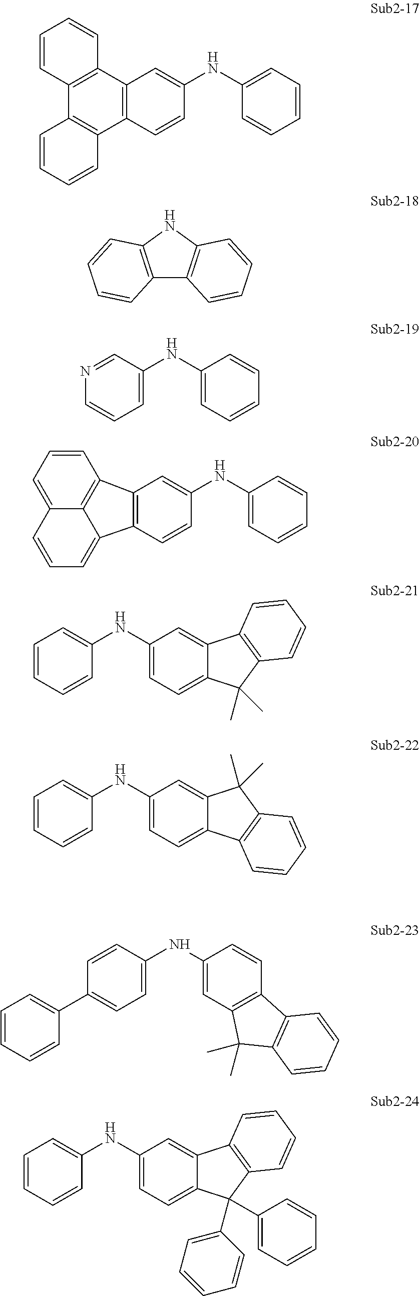

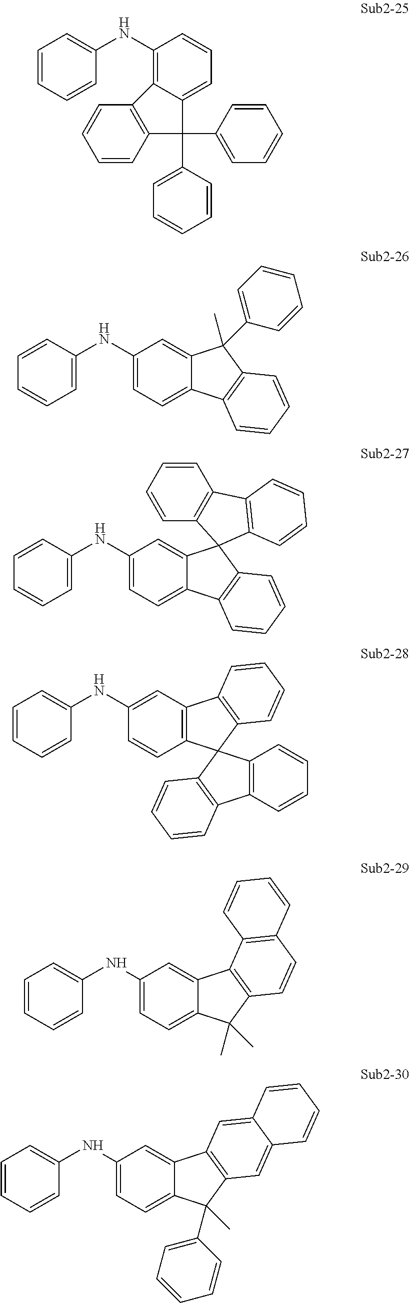

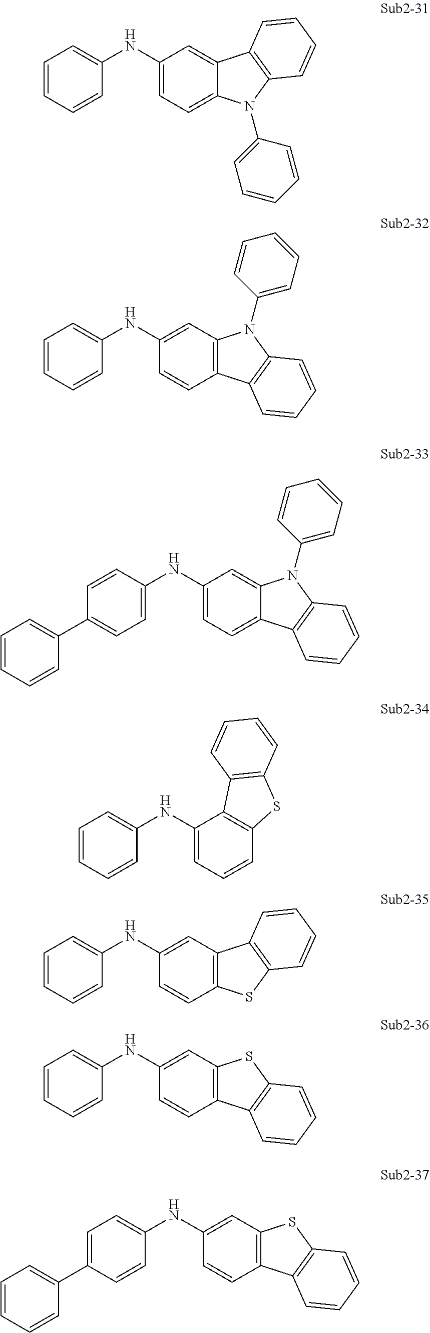

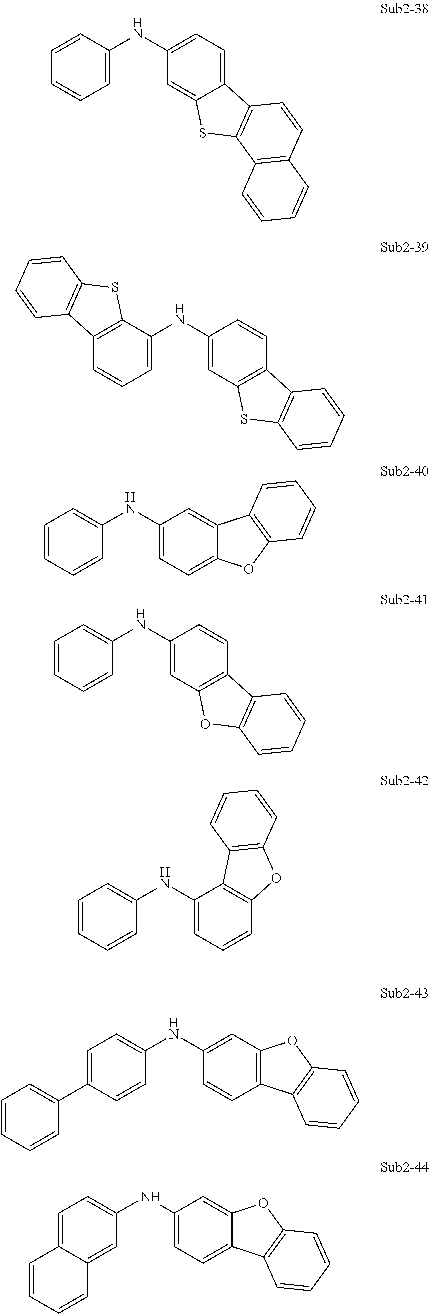

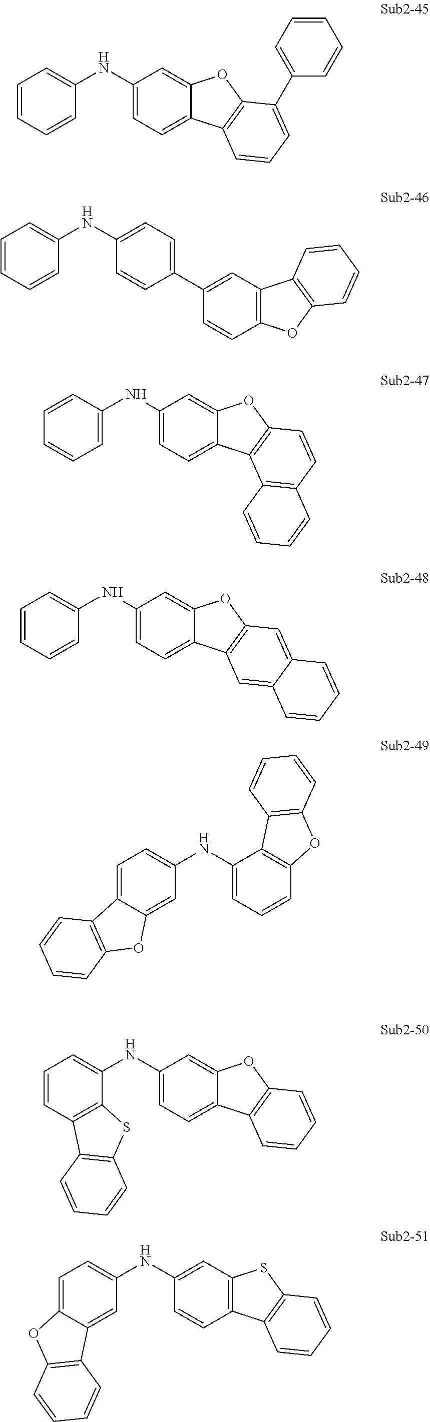

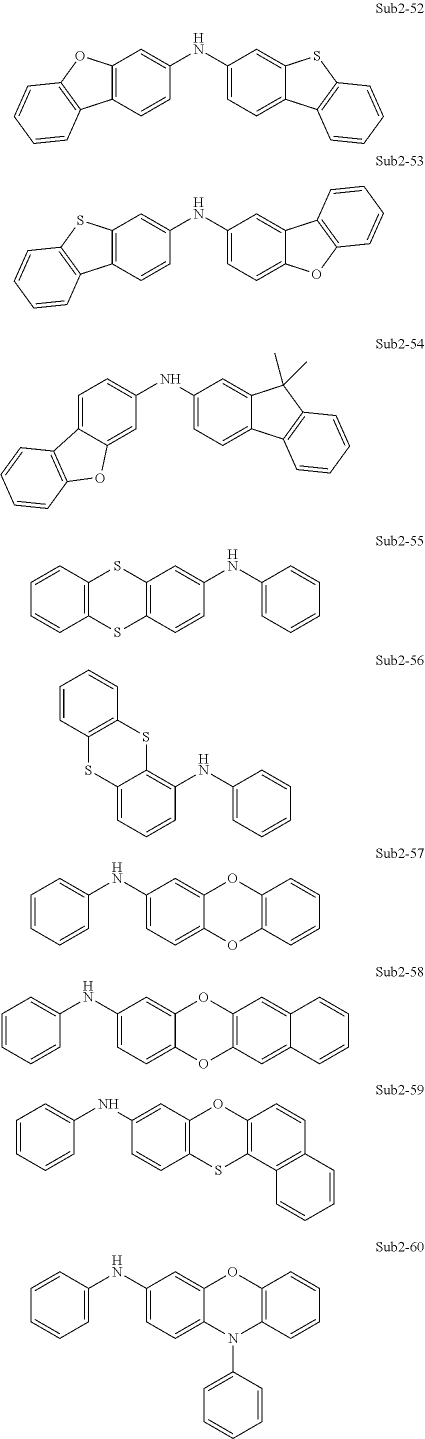

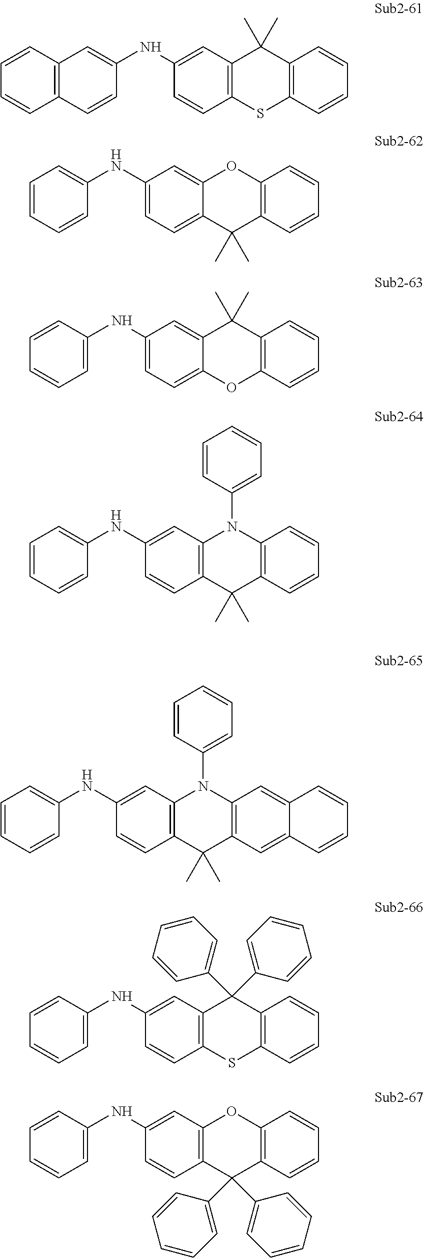

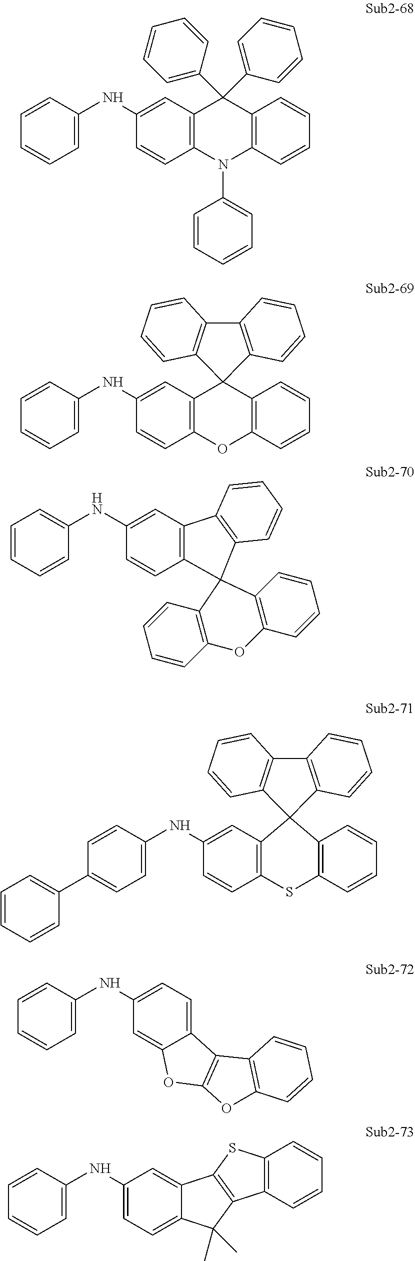

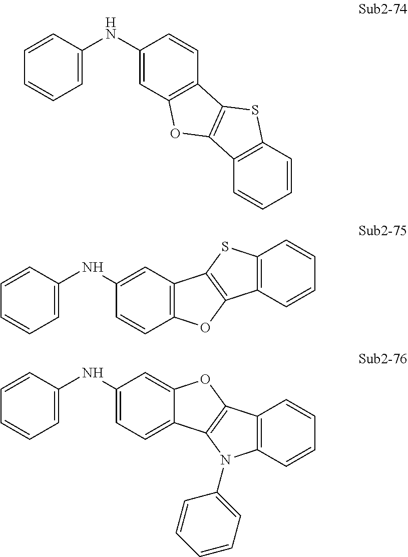

Table 2 shows ED-MS (Field Desorption-Mass Spectrometry) values of compounds belonging to Sub 2.

TABLE 2

| com- | |
|---|---|
| pound | FD-MS |
| Sub 2-1 | m/z = 169.09(C₁₂H₁₁N = 169.23) |
| Sub 2-2 | m/z = 194.08(C₁₃H₁₀N₂ = 194.24) |
| Sub 2-3 | m/z = 174.12(C₁₂H₆D₅N = 174.26) |
| Sub 2-4 | m/z = 245.12(C₁₈H₁₅N = 245.33) |
| Sub 2-5 | m/z = 245.12(C₁₈H₁₅N = 245.33) |
| Sub 2-6 | m/z = 321.15(C₂₄H₁₉N = 321.42) |
| Sub 2-7 | m/z = 245.12(C₁₈H₁₅N = 245.33) |
| Sub 2-8 | m/z = 321.15(C₂₄H₁₉N = 321.42) |
| Sub 2-9 | m/z = 321.15(C₂₄H₁₉N = 321.42) |
| Sub 2-10 | m/z = 295.14(C₂₂H₁₇N = 295.39) |
| Sub 2-11 | m/z = 295.14(C₂₂H₁₇N = 295.39) |
| Sub 2-12 | m/z = 321.15(C₂₄H₁₉N = 321.42) |
| Sub 2-13 | m/z = 219.1(C₁₆H₁₃N = 219.29) |
| Sub 2-14 | m/z = 219.1(C₁₆H₁₃N = 219.29) |
| Sub 2-15 | m/z = 269.12(C₂₀H₁₅N = 269.35) |
| Sub 2-16 | m/z = 269.12(C₂₀H₁₅N = 269.35) |
| Sub 2-17 | m/z = 319.14(C₂₄H₁₇N = 319.41) |
| Sub 2-18 | m/z = 167.07(C₁₂H₉N = 167.21) |
| Sub 2-19 | m/z = 170.08(C₁₁H₁₀N₂ = 170.22) |
| Sub 2-20 | m/z = 293.12(C₂₂H₁₅N = 293.37) |
| Sub 2-21 | m/z = 285.15(C₂₁H₁₉N = 285.39) |
| Sub 2-22 | m/z = 285.15(C₂₁H₁₉N = 285.39) |
| Sub 2-23 | m/z = 361.18(C₂₇H₂₃N = 361.49) |
| Sub 2-24 | m/z = 409.18(C₅₁H₂₃N = 409.53) |
| Sub 2-25 | m/z = 409.18(C₅₁H₂₃N = 409.53) |
| Sub 2-26 | m/z = 347.17(C₂₆H₂₁N = 347.46) |
| Sub 2-27 | m/z = 407.17(C₃₁H₂₁N = 407.52) |
| Sub 2-28 | m/z = 407.17(C₃₁H₂₁N = 407.52) |
| Sub 2-29 | m/z = 335.17(C₂₅H₂₁N = 335.45) |
| Sub 2-30 | m/z = 397.18(C₃₀H₂₃N = 397.52) |
| Sub 2-31 | m/z = 334.15(C₂₄H₁₈N₂ = 334.42) |
| Sub 2-32 | m/z = 334.15(C₂₄H₁₈N₂ = 334.42) |
| Sub 2-33 | m/z = 410.18(C₃₀H₂₂N₂ = 410.52) |
| Sub 2-34 | m/z = 275.08(C₁₈H₁₃NS = 275.37) |
| Sub 2-35 | m/z = 275.08(C₁₈H₁₃NS = 275.37) |
| Sub 2-36 | m/z = 275.08(C₁₈H₁₃NS = 275.37) |
| Sub 2-37 | m/z = 351.11(C₂₄H₁₇NS = 351.47) |
| Sub 2-38 | m/z = 325.09(C₂₂H₁₅NS = 325.43) |
| Sub 2-39 | m/z = 381.06(C₂₄H₁₅NS₂ = 381.51) |
| Sub 2-40 | m/z = 259.1(C₁₈H₁₃NO = 259.31) |
| Sub 2-41 | m/z = 259.1(C₁₈H₁₃NO = 259.31) |
| Sub 2-42 | m/z = 259.1(C₁₈H₁₃NO = 259.31) |
| Sub 2-43 | m/z = 335.13(C₂₄H₁₇NO = 335.41) |
| Sub 2-44 | m/z = 309.12(C₂₂H₁₅NO = 309.37) |
| Sub 2-45 | m/z = 335.13(C₂₄H₁₇NO = 335.41) |
| Sub 2-46 | m/z = 335.13(C₂₄H₁₇NO = 335.41) |
| Sub 2-47 | m/z = 309.12(C₂₂H₁₅NO = 309.37) |
| Sub 2-48 | m/z = 309.12(C₂₂H₁₅NO = 309.37) |
| Sub 2-49 | m/z = 349.11(C₂₄H₁₅NO₂ = 349.39) |
| Sub 2-50 | m/z = 365.09(C₂₄H₁₅NOS = 365.45) |
| Sub 2-51 | m/z = 365.09(C₂₄H₁₅NOS = 365.45) |
| Sub 2-52 | m/z = 365.09(C₂₄H₁₅NOS = 365.45) |
| Sub 2-53 | m/z = 365.09(C₂₄H₁₅NOS = 365.45) |
| Sub 2-54 | m/z = 375.16(C₂₇H₂₁NO = 375.47) |
| Sub 2-55 | m/z = 307.05(C₁₈H₁₃NS₂ = 307.43) |
| Sub 2-56 | m/z = 307.05(C₁₈H₁₃NS₂ = 307.43) |
| Sub 2-57 | m/z = 275.09(C₁₈H₁₃NO₂ = 275.31) |
| Sub 2-58 | m/z = 325.11(C₂₂H₁₅NO₂ = 325.37) |
| Sub 2-59 | m/z = 341.09(C₂₂H₁₅NOS = 341.43) |
| Sub 2-60 | m/z = 350.14(C₂₄H₁₈N₂O = 350.42) |
| Sub 2-61 | m/z = 367.14(C₂₅H₂₁NS = 367.51) |
| Sub 2-62 | m/z = 301.15(C₂₁H₁₉NO = 301.39) |
| Sub 2-63 | m/z = 301.15(C₂₁H₁₉NO = 301.39) |
| Sub 2-64 | m/z = 376.19(C₂₇H₂₄N₂ = 376.5) |
| Sub 2-65 | m/z = 426.21(C₃₁H₂₆N2 = 426.56) |
| Sub 2-66 | m/z = 441.16(C₃₁H₂₃NS = 441.59) |
| Sub 2-67 | m/z = 425.18(C₃₁H₂₃NO = 425.53) |
| Sub 2-68 | m/z = 500.23(C₃₇H₂₈N₂ = 500.65) |
| Sub 2-69 | m/z = 423.16(C₃₁H₂₁NO = 423.52) |
| Sub 2-70 | m/z = 423.16(C₃₁H₂₁NO = 423.52) |
| Sub 2-71 | m/z = 515.17(C₃₇H₂₅NS = 515.67) |
| Sub 2-72 | m/z = 299.09(C₂₀H₁₃N0₂ = 299.33) |
| Sub 2-73 | m/z = 341.12(C₂₃H₁₉NS = 341.47) |
| Sub 2-74 | m/z = 315.07(C₂₀H₁₃NOS = 315.39) |
| Sub 2-75 | m/z = 315.07(C₂₀H₁₃NOS = 315.39) |
| Sub 2-76 | m/z = 374.14(C₂₆H₁₈N₂O = 374.44) |

### III. Synthesis Example of Final Product 1

### Synthesis Example 1-1

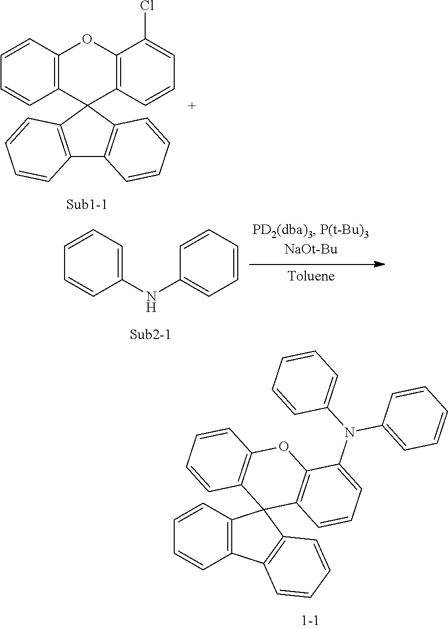 [SMILES: Clc1cccc2c1Oc1ccccc1C21c2ccccc2-c2ccccc21 ; c1ccc(Nc2ccccc2)cc1 ; c1ccc(N(c2ccccc2)c2cccc3c2Oc2ccccc2C32c3ccccc3-c3ccccc32)cc1]

After adding Sub 1-1 (10 g, 27.26 mmol) to a round-bottom flask and dissolving with toluene (300 mL), Sub 2-1 (5.07 g, 29.99 mmol), Pd₂(dba)₃ (1.25 g, 1.36 mmol), P(t-Bu)₃ (0.55 g, 2.73 mmol), NaOt-Bu (7.86 g, 81.78 mmol) were added and stirred at 100° C. When the reaction was completed, the mixture was extracted with CH₂Cl₂ and water, the organic layer was dried over MgSO₄, concentrated, and the resulting compound was recrystallized using silicagel column to obtain 11.7 g (yield 86%) of the product.

### Synthesis Example 1-15

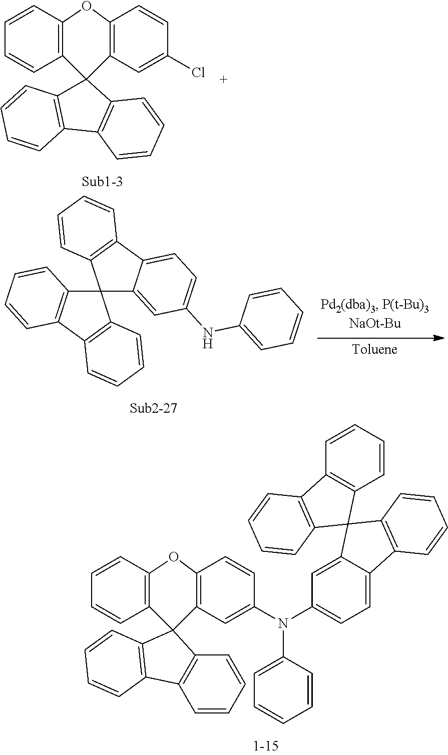 [SMILES: Clc1ccc2c(c1)C1(c3ccccc3O2)c2ccccc2-c2ccccc21 ; c1ccc(Nc2ccc3c(c2)C2(c4ccccc4-c4ccccc42)c2ccccc2-3)cc1 ; c1ccc(N(c2ccc3c(c2)C2(c4ccccc4O3)c3ccccc3-c3ccccc32)c2ccc3c(c2)C2(c4ccccc4-c4ccccc42)c2ccccc2-3)cc1]

Sub 1-3 (10 g, 27.26 mmol), Toluene (500 mL), Sub 2-27 (12.22 g, 29.99 mmol), Pd₂(dba)₃ (1.25 g, 1.36 mmol), P(t-Bu)₃ (0.55 g, 2.73 mmol), NaOt-Bu (7.86 g, 81.78 mmol) were carried out in Synthesis example 1-1 to obtain the product (15.89 g, 79%).

### Synthesis Example 1-27

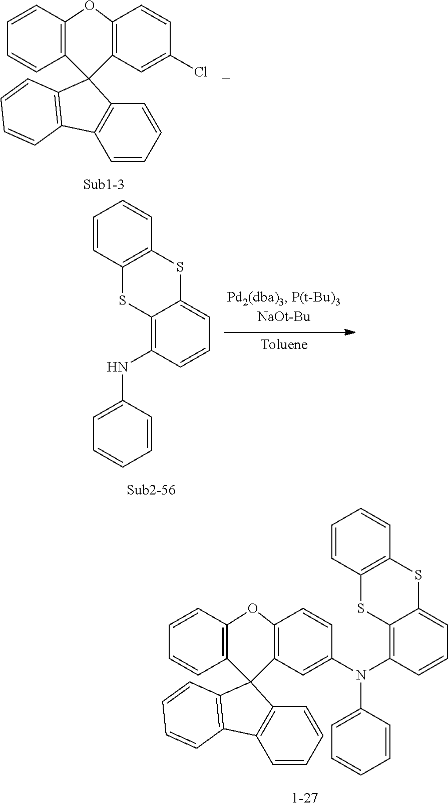

Sub 1-3 (10 g, 27.26 mmol), Toluene (500 mL), Sub 2-56 (9.22 g, 29.99 mmol), Pd₂(dba)₃ (1.25 g, 1.36 mmol), P(t-Bu)₃ (0.55 g, 2.73 mmol), NaOt-Bu (7.86 g, 81.78 mmol) were carried out in Synthesis example 1-1 to obtain the product (14.6 g, 84%).

### Synthesis Example 1-42

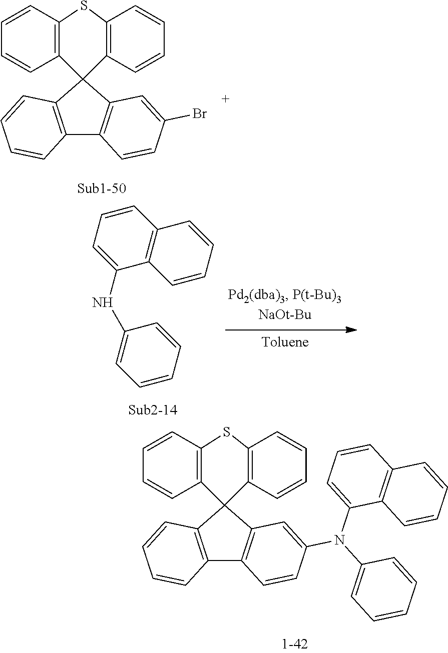 [SMILES: Brc1ccc2c(c1)C1(c3ccccc3Sc3ccccc31)c1ccccc1-2 ; c1ccc(Nc2cccc3ccccc23)cc1 ; c1ccc(N(c2ccc3c(c2)C2(c4ccccc4Sc4ccccc42)c2ccccc2-3)c2cccc3ccccc23)cc1]

Sub 1-50 (10 g, 23.40 mmol), Toluene (500 mL), Sub 2-14 (5.64 g, 25.74 mmol), Pd₂(dba)₃ (1.25 g, 1.36 mmol), P(t-Bu)₃ (0.55 g, 2.73 mmol), NaOt-Bu (7.86 g, 81.78 mmol) were carried out in Synthesis example 1-1 to obtain the product (10.85 g, 82%).

### Synthesis Example 1-74

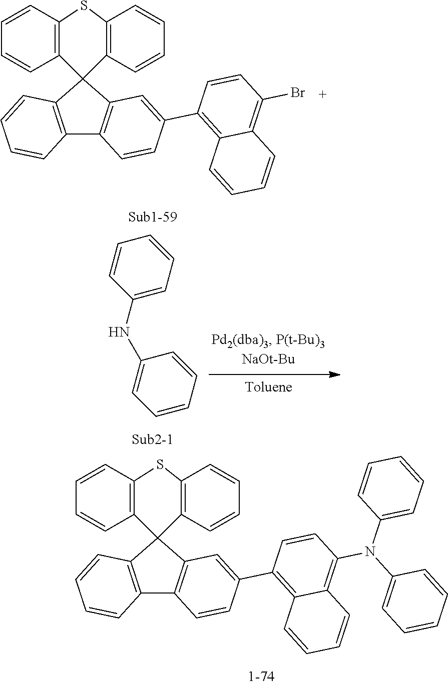 [SMILES: Brc1ccc(-c2ccc3c(c2)C2(c4ccccc4Sc4ccccc42)c2ccccc2-3)c2ccccc12 ; c1ccc(Nc2ccccc2)cc1 ; c1ccc(N(c2ccccc2)c2ccc(-c3ccc4c(c3)C3(c5ccccc5Sc5ccccc53)c3ccccc3-4)c3ccccc23)cc1]

Sub 1-59 (10 g, 18.07 mmol), Toluene (500 mL), Sub 2-1 (3.36 g, 19.87 mmol), Pd₂(dba)₃ (1.25 g, 1.36 mmol), P(t-Bu)₃ (0.55 g, 2.73 mmol), NaOt-Bu (7.86 g, 81.78 mmol) were carried out in Synthesis example 1-1 to obtain the product (9.9 g, 85%).

### Synthesis Example 1-103

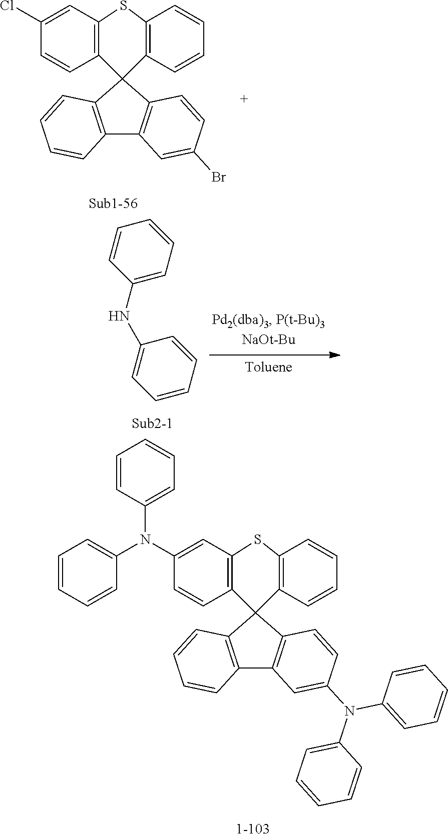

Sub 1-56 (10 g, 21.65 mmol), Toluene (500 mL), Sub 2-1 (4.03 g, 23.82 mmol), Pd₂(dba)₃ (1.25 g, 1.36 mmol), P(t-Bu)₃ (0.55 g, 2.73 mmol), NaOt-Bu (7.86 g, 81.78 mmol) were carried out in Synthesis example 1-1 to obtain the product (12.63 g, 90%).

### Synthesis Example 1-126

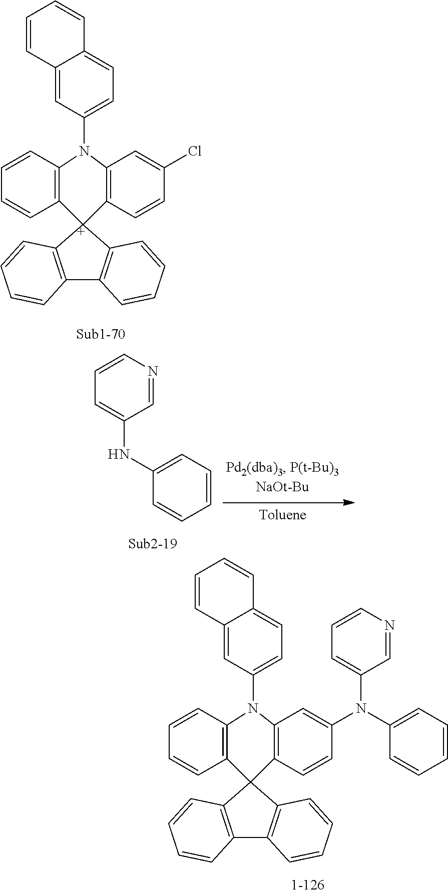

Sub 1-70 (10 g, 20.32 mmol), Toluene (500 mL), Sub 2-19 (3.81 g, 22.36 mmol), Pd₂(dba)₃ (1.25 g, 1.36 mmol), P(t-Bu)₃ (0.55 g, 2.73 mmol), NaOt-Bu (7.86 g, 81.78 mmol) were carried out in Synthesis example 1-1 to obtain the product (11.28 g, 89%).

### Synthesis Example 1-142

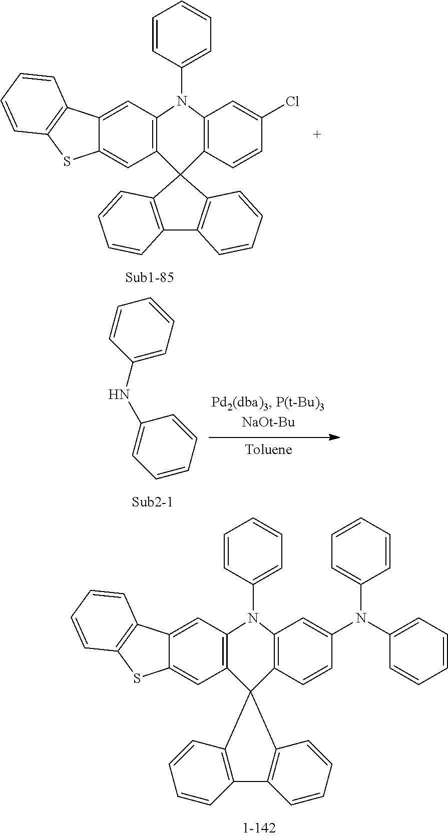

Sub 1-85 (10 g, 18.24 mmol), Toluene (500 mL), Sub 2-1 (3.4 g, 20.07 mmol), Pd₂(dba)₃ (1.25 g, 1.36 mmol), P(t-Bu)₃ (0.55 g, 2.73 mmol), NaOt-Bu (7.86 g, 81.78 mmol) were carried out in Synthesis example 1-1 to obtain the product (9.44 g, 76%).

Table 3 shows ED-MS (Field Desorption-Mass Spectrometry) values of compounds belonging to Final product 1.

TABLE 3

| com- | |
|---|---|
| pound | FD-MS |
| 1-1 | m/z = 499.19(C₃₇H₂₅NO = 499.61) |
| 1-2 | m/z = 499.19(C₃₇H₂₅NO = 499.61) |
| 1-3 | m/z = 499.19(C₃₇H₂₅NO = 499.61) |
| 1-4 | m/z = 499.19(C₃₇H₂₅NO = 499.61) |
| 1-5 | m/z = 575.22(C₄₃H₂₉NO = 575.71) |
| 1-6 | m/z = 589.2(C₄₃H₂₇NO₂ = 589.69) |
| 1-7 | m/z = 549.21(C₄₁H₂₇NO = 549.67) |
| 1-8 | m/z = 651.26(C₄₉H₃₃NO = 651.81) |
| 1-9 | m/z = 599.22(C₄₅H₂₉NO = 599.73) |
| 1-10 | m/z = 605.18(C₄₃H₂₇NOS = 605.76) |
| 1-11 | m/z = 615.26(C₄₆H₃₃NO = 615.78) |
| 1-12 | m/z = 565.19(C₄₁H₂₇NS = 565.73) |
| 1-13 | m/z = 695.19(C₄₉H₂₉NO₂S = 695.84) |
| 1-14 | m/z = 755.26(C₅₆H₃₇NS = 755.98) |
| 1-15 | m/z = 737.27(C₅₆H₃₅NO = 737.9) |
| 1-16 | m/z = 671.17(C₄₇H₂₉NS₂ = 671.88) |
| 1-17 | m/z = 639.22(C₄₇H₂₉NO₂ = 639.75) |
| 1-18 | m/z = 681.25(C₅₀H₃₅NS = 681.9) |
| 1-19 | m/z = 574.24(C₄₃H₃₀N₂ = 574.73) |
| 1-20 | m/z = 739.3(C₅₅H₃₇N₃ = 739.92) |
| 1-21 | m/z = 639.22(C₄₇H₂₉NO₂ = 639.75) |
| 1-22 | m/z = 664.25(C₄₉H₃₂N₂O = 664.81) |
| 1-23 | m/z = 605.2(C₄₃H₂₇NO₃ = 605.69) |
| 1-24 | m/z = 743.26(C₅₅H₃₇NS = 743.97) |
| 1-25 | m/z = 720.22(C₅₁H₃₂N₂OS = 720.89) |
| 1-26 | m/z = 687.21(C₄₈H₃₃NS₂ = 687.92) |
| 1-27 | m/z = 637.15(C₄₃H₂₇NOS₂ = 637.82) |
| 1-28 | m/z = 629.2(C₄₅H₂₇NO₃ = 629.72) |
| 1-29 | m/z = 704.25(C₅₁H₃₂N₂O₂ = 704.83) |
| 1-30 | m/z = 647.23(C₄₆H₃₃NOS = 647.84) |
| 1-31 | m/z = 650.27(C₄₉H₃₄N₂ = 650.83) |
| 1-32 | m/z = 650.27(C₄₉H₃₄N₂ = 650.83) |
| 1-33 | m/z = 575.22(C₄₃H₂₉NO = 575.71) |
| 1-34 | m/z = 641.22(C₄₇H₃₁NS = 641.83) |
| 1-35 | m/z = 665.24(C₄₉H₃₁NO₂ = 665.79) |
| 1-36 | m/z = 682.24(C₄₉H₃₁FN₂O = 682.8) |
| 1-37 | m/z = 641.22(C₄₇H₃₁NS = 641.83) |
| 1-38 | m/z = 681.21(C₄₉H₃₁NOS = 681.85) |
| 1-39 | m/z = 665.24(C₄₉H₃₁NO₂ = 665.79) |
| 1-40 | m/z = 726.3(C₅₅H₃₈N₂ = 726.92) |
| 1-41 | m/z = 499.19(C₃₇H₂₅NO = 499.61) |
| 1-42 | m/z = 565.19(C₄₁H₂₇NS = 565.73) |
| 1-43 | m/z = 575.22(C₄₃H₂₉NO = 575.71) |
| 1-44 | m/z = 605.18(C₄₃H₂₇NOS = 605.76) |
| 1-45 | m/z = 631.23(C₄₆H₃₃NS = 631.84) |
| 1-46 | m/z = 667.23(C₄₉H₃₃NS = 667.87) |
| 1-47 | m/z = 599.22(C₄₅H₂₉NO = 599.73) |
| 1-48 | m/z = 695.19(C₄₉H₂₉NO₂S = 695.84) |
| 1-49 | m/z = 639.22(C₄₇H₂₉NO₂ = 639.75) |
| 1-50 | m/z = 641.22(C₄₇H₃₁NS = 641.83) |
| 1-51 | m/z = 649.24(C₄₉H₃₁NO = 649.79) |
| 1-52 | m/z = 639.2(C₄₇H₂₉NS = 639.82) |
| 1-53 | m/z = 705.27(C₅₂H₃₅NO₂ = 705.86) |
| 1-54 | m/z = 707.26(C₅₂H₃₇NS = 707.94) |
| 1-55 | m/z = 739.29(C₅₆H₃₇NO = 739.92) |
| 1-56 | m/z = 737.27(C₅₆H₃₅NO = 737.9) |
| 1-57 | m/z = 740.28(C₅₅H₃₆N₂O = 740.91) |
| 1-58 | m/z = 681.21(C₄₉H₃₁NOS = 681.85) |
| 1-59 | m/z = 631.25(C₄₆H₃₃NO₂ = 631.78) |
| 1-60 | m/z = 753.27(C₅₆H₃₅NO₂ = 753.9) |
| 1-61 | m/z = 845.28(C₆₂H₃₉NOS = 846.06) |
| 1-62 | m/z = 753.27(C₅₆H₃₅NO₂ = 753.9) |
| 1-63 | m/z = 605.2(C₄₃H₂₇NO₃ = 605.69) |
| 1-64 | m/z = 706.3(C₅₂H₃₈N₂O = 706.89) |
| 1-65 | m/z = 771.26(C₅₆H₃₇NOS = 771.98) |
| 1-66 | m/z = 755.28(C₅₆H₃₇NO₂ = 755.92) |
| 1-67 | m/z = 655.21(C₄₇H₂₉NO₃ = 655.75) |
| 1-68 | m/z = 772.29(C₅₆H₄₀N₂S = 773.01) |
| 1-69 | m/z = 697.24(C₅₀H₃₅NOS = 697.9) |
| 1-70 | m/z = 687.17(C₄₇H₂₉NOS₂ = 687.88) |
| 1-71 | m/z = 680.25(C₄₉H₃₂N₂O₂ = 680.81) |
| 1-72 | m/z = 645.18(C₄₅H₂₇NO₂S = 645.78) |
| 1-73 | m/z = 830.33(C₆₂H₄₂N₂O = 831.03) |
| 1-74 | m/z = 641.22(C₄₇H₃₁NS = 641.83) |
| 1-75 | m/z = 665.24(C₄₉H₃₁NO₂ = 665.79) |
| 1-76 | m/z = 681.21(C₄₉H₃₁NOS = 681.85) |
| 1-77 | m/z = 665.24(C₄₉H₃₁NO₂ = 665.79) |
| 1-78 | m/z = 625.24(C₄₇H₃₁NO = 625.77) |
| 1-79 | m/z = 504.22(C₃₇H₂₀D₅NO = 504.64) |
| 1-80 | m/z = 631.29(C₄₇H₃₇NO = 631.82) |
| 1-81 | m/z = 720.19(C₅₀H₂₈N₂0₂S = 720.85) |
| 1-82 | m/z = 539.22(C₄₀H₂₉NO = 539.68) |
| 1-83 | m/z = 529.2(C₃₈H₂₇NO₂ = 529.64) |
| 1-84 | m/z = 787.2(C₅₅H₃₃NOS₂ = 788) |
| 1-85 | m/z = 575.22(C₄₃H₂₉NO = 575.71) |
| 1-86 | m/z = 651.26(C₄₉H₃₃NO = 651.81) |
| 1-87 | m/z = 576.22(C₄₂H₂₈N₂O = 576.7) |
| 1-88 | m/z = 575.22(C₄₃H₂₉NO = 575.71) |
| 1-89 | m/z = 503.22(C₃₇H₂₁D₄NO = 503.64) |
| 1-90 | m/z = 742.3(C₅₅H₃₈N₂O = 742.92) |
| 1-91 | m/z = 772.25(C₅₅H₃₆N₂OS = 772.97) |
| 1-92 | m/z = 716.28(C₅₃H₃₆N₂O = 716.88) |
| 1-93 | m/z = 766.31(C₅₆H₃₈N₄ = 766.95) |
| 1-94 | m/z = 772.25(C₅₅H₃₆N₂OS = 772.97) |
| 1-95 | m/z = 858.36(C₆₄H₄₆N₂O = 859.09) |
| 1-96 | m/z = 862.27(C₆₁H₃₈N₂O₂S = 863.05) |
| 1-97 | m/z = 847.3(C₆₁H₄₁N₃S = 848.08) |
| 1-98 | m/z = 831.32(C₆₁H₄₁N₃O = 832.02) |
| 1-99 | m/z = 844.35(C₆₃H₄₄N₂O = 845.06) |
| 1-100 | m/z = 1042.29(C₇₃H₄₂N₂O₄S = 1043.21) |
| 1-101 | m/z = 920.32(C₆₈H₄₄N₂S = 921.17) |
| 1-102 | m/z = 879.27(C₆₁H₄₁N₃S₂ = 880.14) |
| 1-103 | m/z = 682.24(C₄₉H₃₄N₂S = 682.89) |
| 1-104 | m/z = 848.29(C₆₁H₄₀N₂OS = 849.06) |
| 1-105 | m/z = 766.3(C₅₇H₃₈N₂O = 766.94) |
| 1-106 | m/z = 817.35(C₆₁H₄₃N₃ = 818.04) |
| 1-107 | m/z = 834.31(C₆₁H₄₂N₂S = 835.08) |
| 1-108 | m/z = 497.18(C₃₇H₂₃NO = 497.6) |
| 1-109 | m/z = 667.23(C₄₉H₃₃NS = 667.87) |
| 1-110 | m/z = 726.3(C₅₅H₃₈N₂ = 726.92) |
| 1-111 | m/z = 727.29(C₅₅H₃₇NO = 727.91) |
| 1-112 | m/z = 609.19(C₄₃H₂₈FNS = 609.76) |
| 1-113 | m/z = 758.28(C₅₅H₃₈N₂S = 758.98) |
| 1-114 | m/z = 817.35(C₆₁H₄₃N₃ = 818.04) |
| 1-115 | m/z = 666.27(C₄₉H₃₄N₂O = 666.82) |
| 1-116 | m/z = 792.31(C₅₉H₄₀N₂O = 792.98) |
| 1-117 | m/z = 792.31(C₅₉H₄₀N₂O = 792.98) |
| 1-118 | m/z = 867.36(C₆₅H₄₅N₃ = 868.1) |
| 1-119 | m/z = 741.31(C₅₅H₃₉N₃ = 741.94) |
| 1-120 | m/z = 864.26(C₆₁H₄₀N₂S₂ = 865.13) |
| 1-121 | m/z = 742.3(C₅₅H₃₈N₂O = 742.92) |
| 1-122 | m/z = 933.41(C₇₀H₅₁N₃ = 934.2) |
| 1-123 | m/z = 867.36(C₆₅H₄₅N₃ = 868.1) |
| 1-124 | m/z = 756.28(C₅₅H₃₆N₂O₂ = 756.91) |
| 1-125 | m/z = 575.22(C₄₃H₂₉NO = 575.71) |
| 1-126 | m/z = 625.25(C₄₆H₃₁N₃ = 625.78) |
| 1-127 | m/z = 729.29(C₅₂H₃₅N₅ = 729.89) |
| 1-128 | m/z = 651.26(C₄₉H₃₃NO = 651.81) |
| 1-129 | m/z = 549.21(C₄₁H₂₇NO = 549.67) |
| 1-130 | m/z = 624.26(C₄₇H₃₂N₂ = 624.79) |
| 1-131 | m/z = 624.26(C₄₇H₃₂N₂ = 624.79) |
| 1-132 | m/z = 641.22(C₄₇H₃₁NS = 641.83) |
| 1-133 | m/z = 681.25(C₅₀H₃₅NS = 681.9) |
| 1-134 | m/z = 674.27(C₅₁H₃₄N₂ = 674.85) |
| 1-135 | m/z = 700.29(C₅₃H₃₆N₂ = 700.89) |
| 1-136 | m/z = 599.22(C₄₅H₂₉NO = 599.73) |
| 1-137 | m/z = 615.2(C₄₅H₂₉NS = 615.79) |
| 1-138 | m/z = 624.26(C₄₇H₃₂N₂ = 624.79) |
| 1-139 | m/z = 639.22(C₄₇H₂₉NO₂ = 639.75) |
| 1-140 | m/z = 599.22(C₄₅H₂₉NO = 599.73) |
| 1-141 | m/z = 605.18(C₄₃H₂₇NOS = 605.76) |
| 1-142 | m/z = 680.23(C₄₉H₃₂N₂S = 680.87) |
| 1-143 | m/z = 695.19(C₄₉H₂NO₂S = 695.84) |
| 1-144 | m/z = 664.25(C₄₉H₃₂N₂O = 664.81) |
| 1-145 | m/z = 664.25(C₄₉H₃₂N₂O = 664.81) |
| 1-146 | m/z = 690.3(C₅₂H₃₈N₂ = 690.89) |
| 1-147 | m/z = 796.29(C₅₈H₄₀N₂S = 797.O3) |
| 1-148 | m/z = 589.2(C₄₃H₂₇NO₂ = 589.69) |
| 1-149 | m/z = 766.3(C₅₇H₃₈N₂O = 766.94) |
| 1-150 | m/z = 739.3(C₅₅H₃₇N₃ = 739.92) |
| 1-151 | m/z = 772.25(C₅₅H₃₆N₂OS = 772.97) |
| 1-152 | m/z = 831.32(C₆₁H₄₁N₃O = 832.02) |

### Synthesis Example 2

The compound represented by Formula 2 according to the present invention (final product 2) may be synthesized by reacting Sub 3 and Sub 4 according to Reaction Scheme 4, but is not limited thereto.

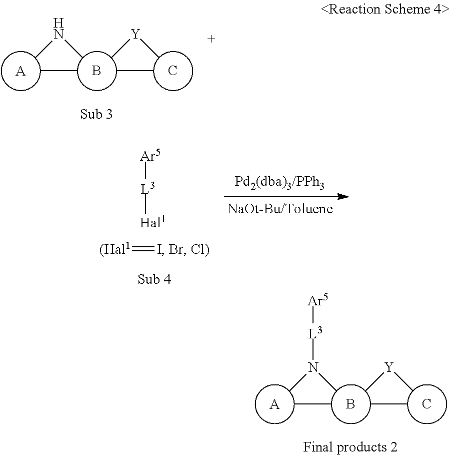

### I. Synthesis Example of Sub 3

Sub 3 of Scheme 4 may be synthesized by the reaction route of Reaction scheme 5, but is not limited thereto.

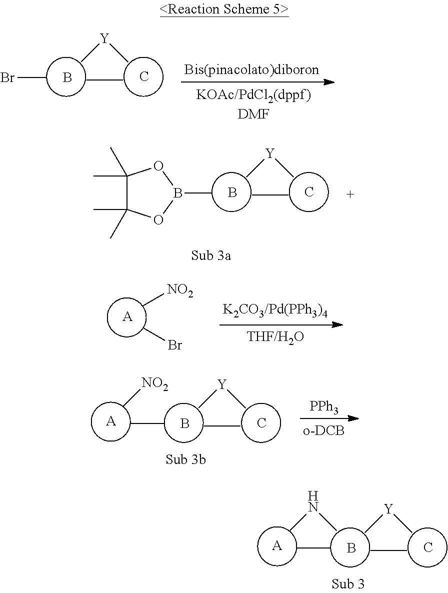

### 1. Synthesis Example of Sub 3-1

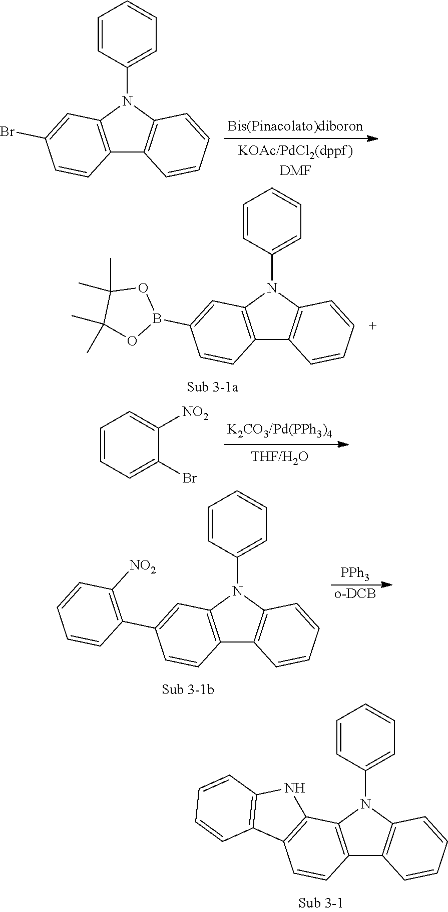

### (1) Synthesis of Sub 3-1a

2-bromo-9-phenyl-9H-carbazole (50 g, 155.18 mmol), bis(pinacolato)diboron (32.17 g, 126.70 mmol), KOAc (45.69 g, 465.54 mmol), PdCl₂(dppf) (3.41 g, 4.66 mmol) were dissolved in DMF (1 L) solvent, and then refluxed at 120° C. for 12 hours. When the reaction was completed, the temperature of the reactant was cooled to room temperature, extracted with CH₂Cl₂, and washed with water. The organic material layer was dried over MgSO4 and concentrated, and the resulting organic material was recrystallized using CH₂Cl₂ and methanol solvent to obtain the desired product (47.03 g, 81%).

### (2) Synthesis of Sub 3-1 b

The obtained Sub 3-1a (46.94 g, 127.12 mmol), 1-bromo-2-nitrobenzene (25.68 g, 127.12 mmol), K₂CO₃ (52.70 g, 381.36 mmol), Pd(PPh₃)₄ (4.41 g, 3.81 mmol) were placed in a round-bottom flask, and then THE (600 mL) and water (300 mL) were added to dissolve, and then refluxed at 80° C. for 12 hours. When the reaction was completed, the temperature of the reactant was cooled to room temperature, extracted with CH₂Cl₂, and washed with water. The organic layer was dried over MgSO₄ and concentrated, and the resulting organic material was separated using a silicagel column to obtain the desired product (31.97 g, 69%).

### (3) Synthesis of Sub 3-1

Sub 3-1b (31.97 g, 87.73 mmol) and triphenylphosphine (57.53 g, 219.33 mmol) obtained above were dissolved in o-dichlorobenzene (500 ml) and refluxed at 200° C. for 24 hours. When the reaction was completed, the solvent was removed by vacuum distillation, and the concentrated product was recrystallized using silicagel column to obtain the desired product (20.42 g, 70%).

### 2. Synthesis Example of Sub 3-16

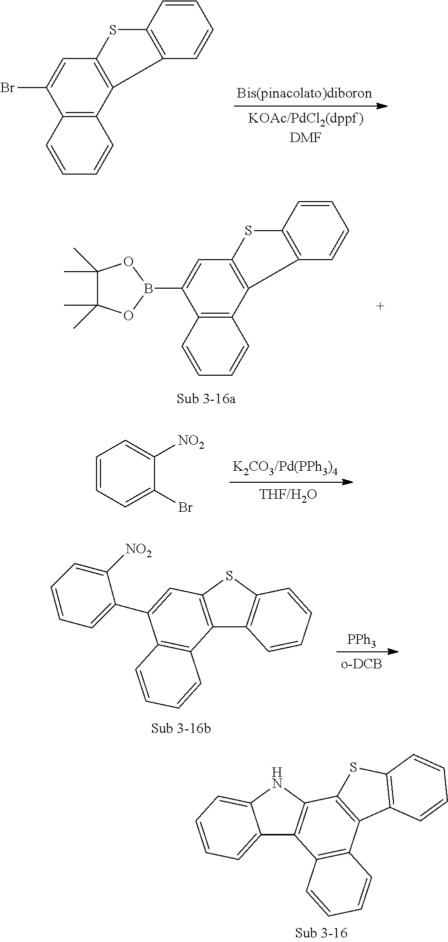

### (1) Synthesis of Sub 3-16a

5-bromobenzo[b]naphtha[1,2-d]thiophene (50 g, 159.64 mmol), bis(pinacolato)diboron (44.59 g, 175.60 mmol), KOAc (47 g, 478.91 mmol), PdCl₂(dppf) (3.50 g, 4.79 mmol) were carried out in the same manner as in the experimental method of Sub 1-1a to obtain a product (46.01 g, 80%).

### (2) Synthesis of Sub 3-16b

The obtained Sub 3-16a (45.94 g, 156.17 mmol), 1-bromo-2-nitrobenzene (38.90 g, 156.17 mmol), K₂CO₃ (64.75 g, 468.51 mmol), Pd(PPh₃)₄ (5.41 g, 4.69 mmol), THE (680 ml), water (340 ml) were carried out in the same manner as in the experimental method of Sub 1-1b to obtain a product (38.85 g, 70%).

### (3) Synthesis of Sub 3-16

The obtained 3-16b (38.85 g, 109.31 mmol) and triphenylphosphine (71.68 g, 273.28 mmol), o-dichlorobenzene (547 mL) were carried out in the same manner as in the experimental method of Sub 3-1 to obtain a product (25.81 g, 73%).

### 3. Synthesis Example of Sub 3-115

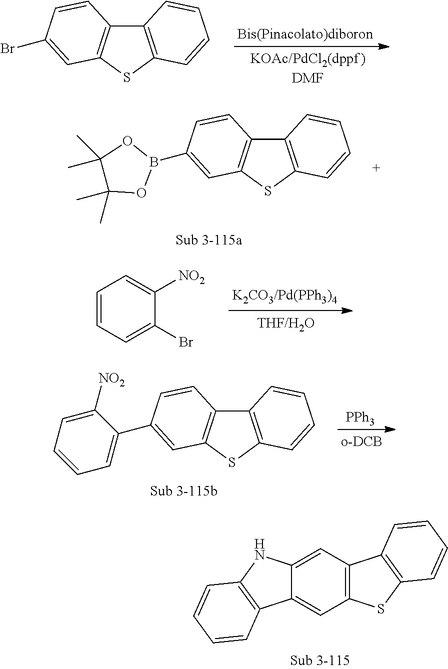

### (1) Synthesis of Sub 3-115a

2-bromodibenzo[b,d]thiophene (50 g, 190 mmol), bis(pinacolato)diboron (53.07 g, 209 mmol), KOAc (55.94 g, 570 mmol), PdCl₂(dppf) (4.17 g, 5.7 mmol) were carried out in the same manner as in the experimental method of Sub 3-1a to obtain a product (46.87 g, 81%).

### (2) Synthesis of Sub 3-115b

The obtained Sub 3-115a (46.87 g, 151.09 mmol), 1-bromo-2-nitrobenzene (30.52 g, 151.09 mmol), K₂CO₃ (62.64 g, 453.27 mmol), Pd(PPh₃)₄ (5.24 g, 4.53 mmol), THE (540 ml), water (270 ml) were carried out in the same manner as in the experimental method of Sub 3-1b to obtain a product (32.68 g, 71%).

### (3) Synthesis of Sub 3-115

The obtained Sub 3-115b (32.68 g, 107.02 mmol), triphenylphosphine (70.18 g, 267.55 mmol), o-dichlorobenzene (466 mL) were carried out in the same manner as in the experimental method of Sub 3-1 to obtain a product (19.83 g, 68%).

### 4. Synthesis Example of Sub 3-160

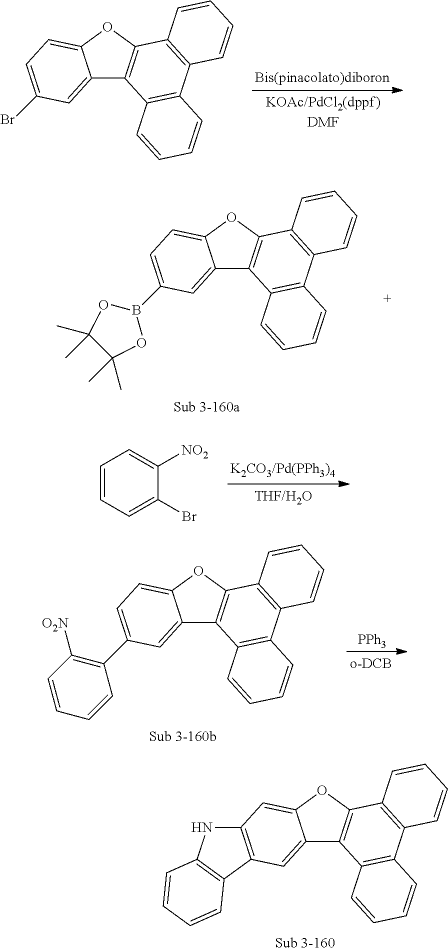 [SMILES: Brc1ccc2oc3c4ccccc4c4ccccc4c3c2c1 ; CC1(C)OB(c2ccc3oc4c5ccccc5c5ccccc5c4c3c2)OC1(C)C ; O=[N+]([O-])c1ccccc1Br ; O=[N+]([O-])c1ccccc1-c1ccc2oc3c4ccccc4c4ccccc4c3c2c1 ; c1ccc2c(c1)[nH]c1cc3oc4c5ccccc5c5ccccc5c4c3cc12 ; "PPh3"]

### (1) Synthesis of Sub 3-160a

12-bromophenanthro[9,10-b]benzofuran (34.2 g, 98.50 mmol), bis(pinacolato)diboron (27.51 g, 108.35 mmol), KOAc (29 g, 295.50 mmol), PdCl₂(dppf) (2.16 g, 2.95 mmol), DMF (621 mL) were carried out in the same manner as in the experimental method of Sub 3-1a to obtain a product (26.02 g, 67%).

### (2) Synthesis of Sub 3-160b

The obtained Sub 3-160a (26 g, 65.94 mmol), 1-bromo-2-nitrobenzene (13.32 g, 65.94 mmol), K₂CO₃ (27.34 g, 197.93 mmol), Pd(PPh₃)₄ (2.29 g, 1.98 mmol), THE (290 ml), water (145 ml) were carried out in the same manner as in the experimental method of Sub 3-1b to obtain a product (20.03 g, 78%).

### (3) Synthesis of Sub 3-160

The obtained Sub 3-160b (19.87 g, 51.03 mmol), triphenylphosphine (33.46 g, 127.56 mmol), o-dichlorobenzene (255 ml) were carried out in the same manner as in the experimental method of Sub 3-1 to obtain a product (7.11 g, 39%).

### 5. Synthesis Example of Sub 3-208

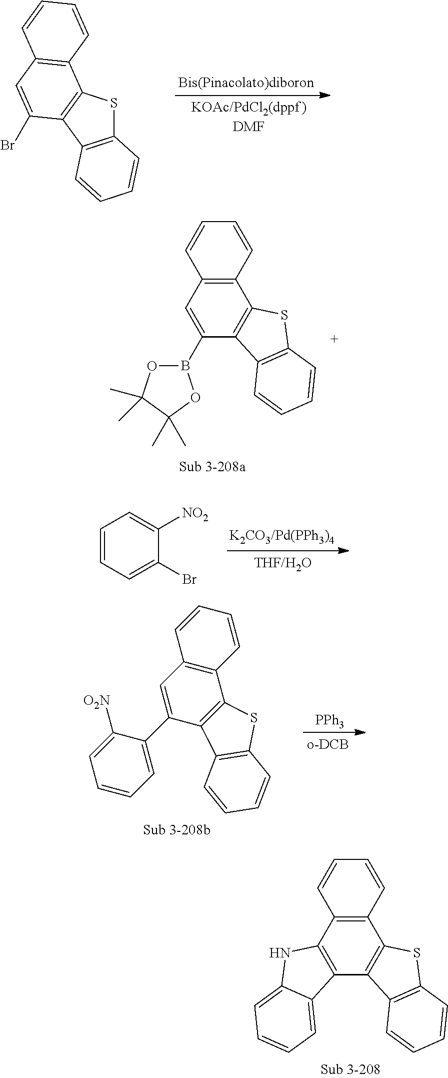

### (1) Synthesis of Sub 3-208a

6-bromobenzo[b]naphtho[2,1-d]thiophene (60 g, 191.56 mmol), bis(pinacolato)diboron (53.51 g, 210.72 mmol), KOAc (56.40 g, 574.69 mmol), PdCl₂(dppf) (4.21 g, 5.75 mmol), DMF (1,207 mL) were carried out in the same manner as in the experimental method of Sub 3-1a to obtain a product (53.14 g, 77%).

### (2) Synthesis of Sub 3-208b

The obtained Sub 3-208a (53.14 g, 147.50 mmol), 1-bromo-2-nitrobenzene (29.80 g, 147.50 mmol), K₂CO₃ (61.16 g, 442.49 mmol), Pd(PPh₃)₄ (5.11 g, 4.42 mmol) were carried out in the same manner as in the experimental method of Sub 3-1b to obtain a product (41.94 g, 80%).

### (3) Synthesis of Sub 3-208

The obtained Sub 3-208b (41.94 g, 118.00 mmol) and triphenylphosphine (77.38 g, 295.01 mmol), o-dichlorobenzene (590 ml) were carried out in the same manner as in the experimental method of Sub 3-1 to obtain a product (19.85 g, 52%).

### 6. Synthesis Example of Sub 3-282

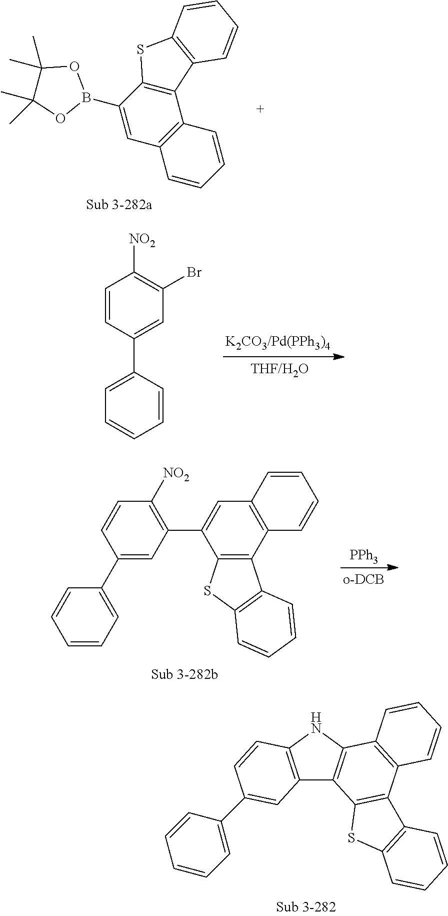 [SMILES: CC1(C)OB(c2cc3ccccc3c3c2sc2ccccc23)OC1(C)C ; O=[N+]([O-])c1ccc(-c2ccccc2)cc1Br ; O=[N+]([O-])c1ccc(-c2ccccc2)cc1-c1cc2ccccc2c2c1sc1ccccc12 ; c1ccc(-c2ccc3[nH]c4c5ccccc5c5c6ccccc6sc5c4c3c2)cc1 ; "PPh3"]

### (1) Synthesis of Sub 3-282b

Sub 3-282a (45 g, 124.90 mmol), 3-bromo-4-nitro-1,1′-biphenyl (35.74 g, 124.90 mmol), K₂CO₃ (51.79 g, 374.71 mmol), Pd(PPh₃)₄ (4.33 g, 3.75 mmol), THE (550 ml), water (275 ml) were carried out in the same manner as in the experimental method of Sub 3-1b to obtain a product (38.27 g, 71%).

### (2) Synthesis of Sub 3-282

The obtained Sub 3-282b (38.27 g, 88.69 mmol), triphenylphosphine (58.16 g, 221.72 mmol), o-dichlorobenzene (443 mL) were carried out in the same manner as in the experimental method of Sub 3-1 to obtain a product (14.53 g, 41%).

Examples of Sub 3 are as follows, but are not limited thereto, and Table 4 shows FD-MS (Field Desorption-Mass Spectrometry) values of compounds belonging to Sub 3.

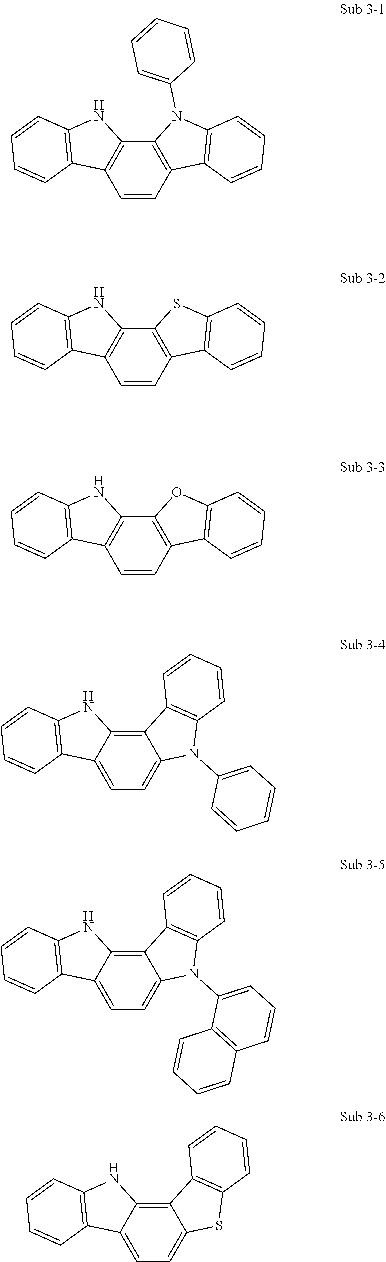

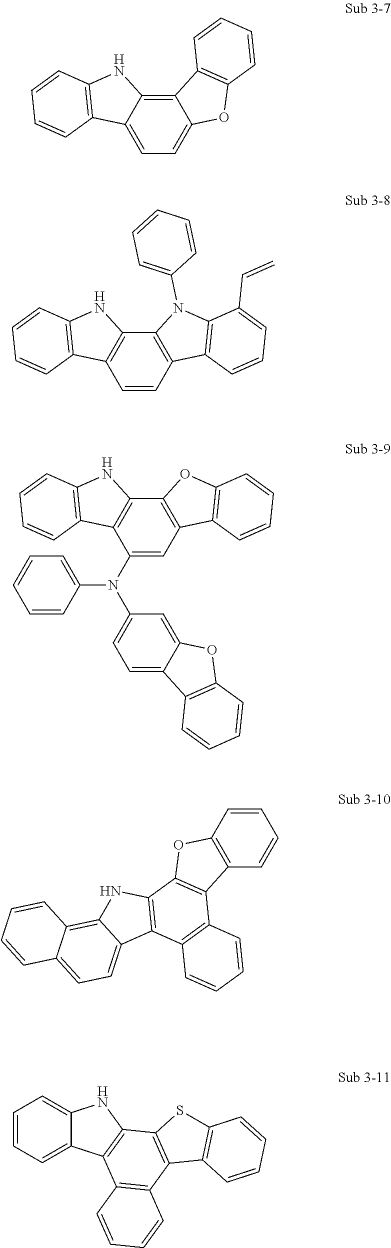 [SMILES: c1ccc2c(c1)[nH]c1c2ccc2oc3ccccc3c21 ; C=Cc1cccc2c3ccc4c5ccccc5[nH]c4c3n(-c3ccccc3)c12 ; c1ccc(N(c2ccc3c(c2)oc2ccccc23)c2cc3c4ccccc4oc3c3[nH]c4ccccc4c23)cc1 ; c1ccc2c(c1)ccc1c2[nH]c2c3oc4ccccc4c3c3ccccc3c12 ; c1ccc2c(c1)[nH]c1c3sc4ccccc4c3c3ccccc3c21]

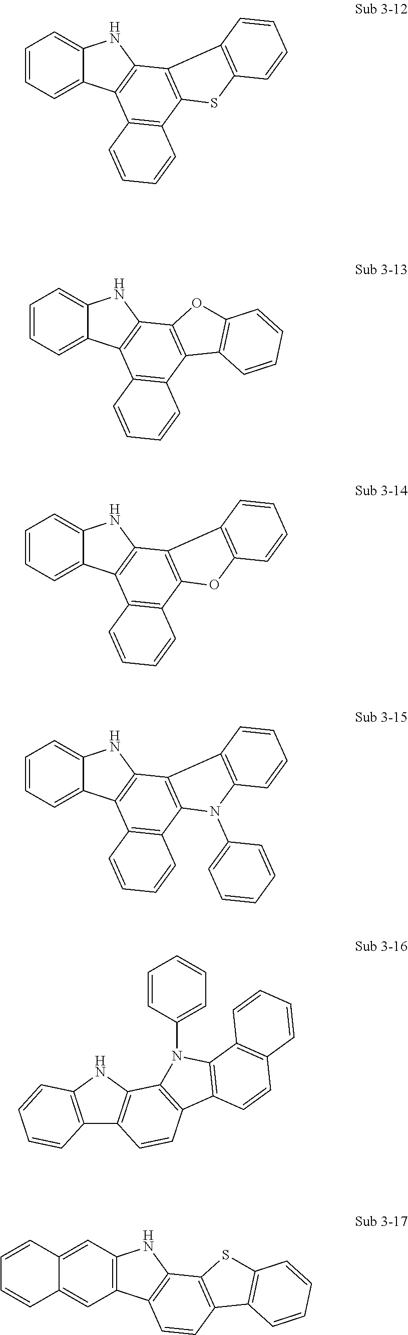 [SMILES: c1ccc2c(c1)[nH]c1c2c2ccccc2c2sc3ccccc3c12 ; c1ccc2c(c1)[nH]c1c3oc4ccccc4c3c3ccccc3c21 ; c1ccc2c(c1)[nH]c1c2c2ccccc2c2oc3ccccc3c12 ; c1ccc(-n2c3ccccc3c3c4[nH]c5ccccc5c4c4ccccc4c32)cc1 ; c1ccc(-n2c3c4ccccc4ccc3c3ccc4c5ccccc5[nH]c4c32)cc1 ; c1ccc2cc3c(cc2c1)[nH]c1c3ccc2c3ccccc3sc21]

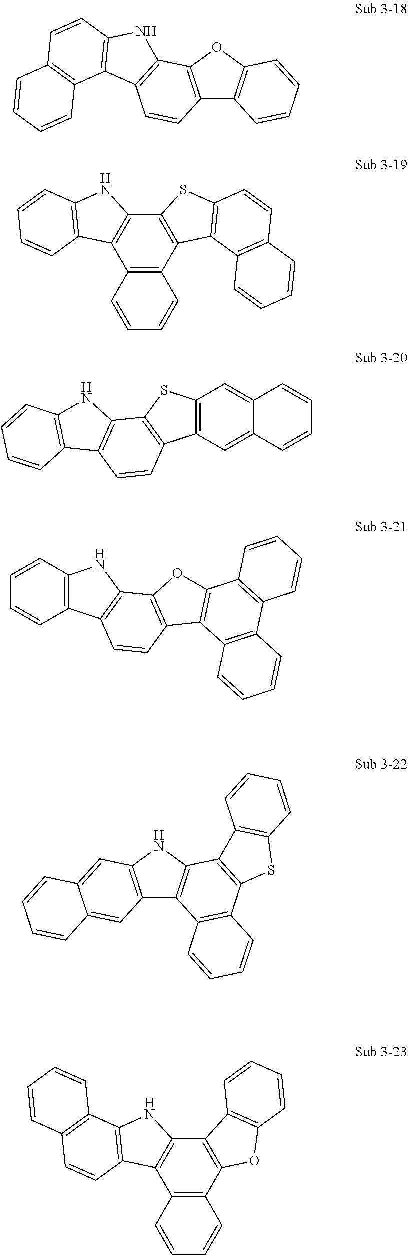 [SMILES: c1ccc2c(c1)ccc1[nH]c3c(ccc4c5ccccc5oc43)c12 ; c1ccc2c(c1)ccc1sc3c4[nH]c5ccccc5c4c4ccccc4c3c12 ; c1ccc2cc3c(cc2c1)sc1c3ccc2c3ccccc3[nH]c21 ; c1ccc2c(c1)[nH]c1c2ccc2c1oc1c3ccccc3c3ccccc3c21 ; c1ccc2cc3c(cc2c1)[nH]c1c3c2ccccc2c2sc3ccccc3c12 ; c1ccc2c(c1)ccc1c2[nH]c2c1c1ccccc1c1oc3ccccc3c21]

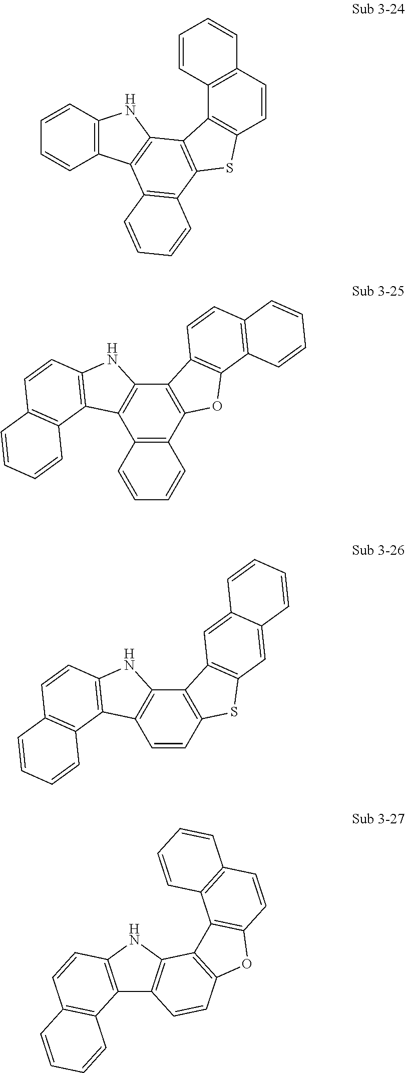 [SMILES: c1ccc2c(c1)ccc1sc3c4ccccc4c4c5ccccc5[nH]c4c3c12 ; c1ccc2c(c1)ccc1c2oc2c3ccccc3c3c([nH]c4ccc5ccccc5c43)c12 ; c1ccc2cc3c(cc2c1)sc1ccc2c([nH]c4ccc5ccccc5c42)c13 ; c1ccc2c(c1)ccc1[nH]c3c(ccc4oc5ccc6ccccc6c5c43)c12]

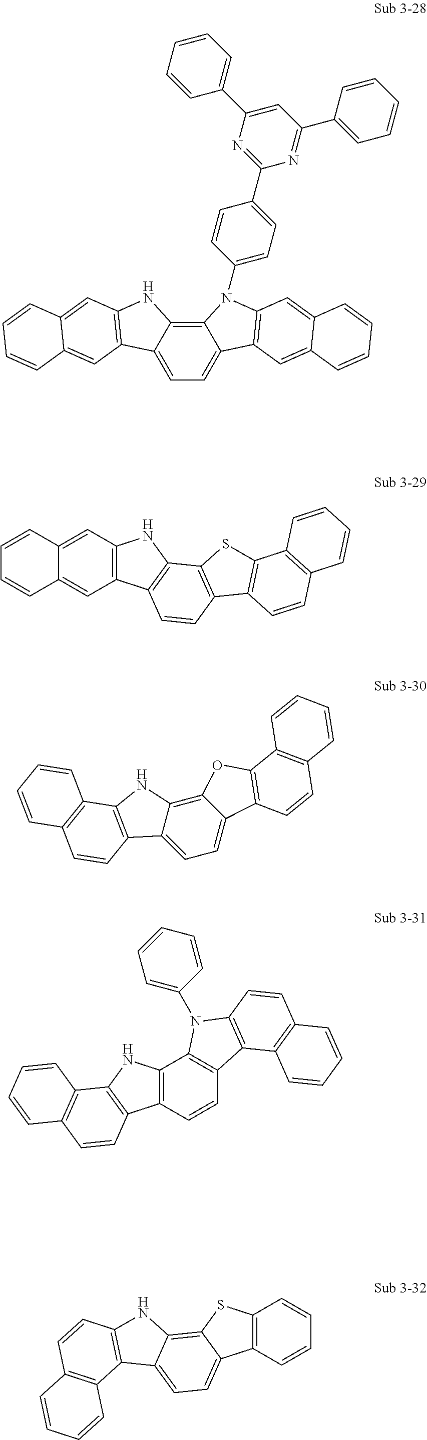 [SMILES: c1ccc(-c2cc(-c3ccccc3)nc(-c3ccc(-n4c5cc6ccccc6cc5c5ccc6c7cc8ccccc8cc7[nH]c6c54)cc3)n2)cc1 ; c1ccc2cc3c(cc2c1)[nH]c1c3ccc2c3ccc4ccccc4c3sc21 ; c1ccc2c(c1)ccc1c3ccc4c5ccc6ccccc6c5oc4c3[nH]c21 ; c1ccc(-n2c3ccc4ccccc4c3c3ccc4c5ccc6ccccc6c5[nH]c4c32)cc1 ; c1ccc2c(c1)ccc1[nH]c3c(ccc4c5ccccc5sc43)c12]

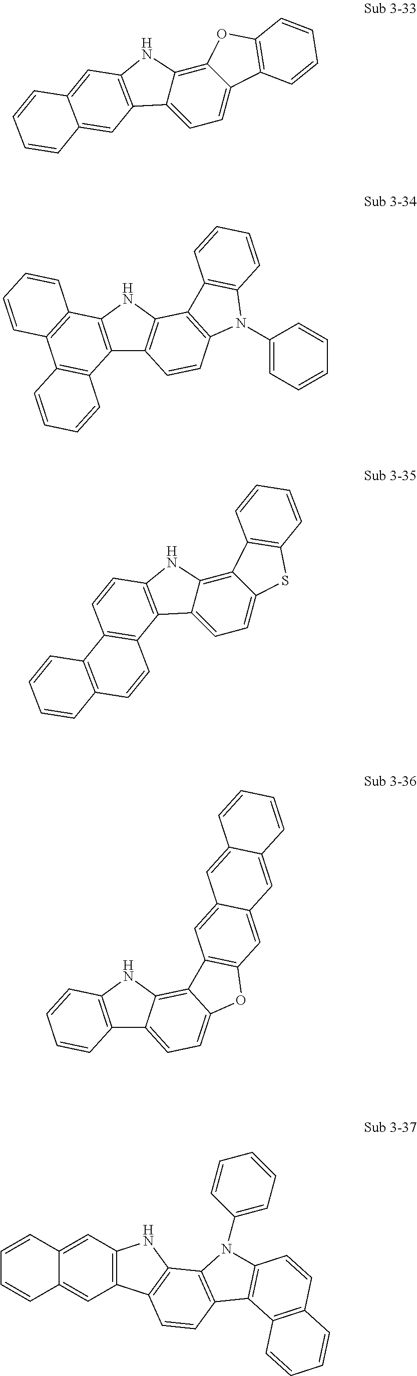 [SMILES: c1ccc2cc3c(cc2c1)[nH]c1c3ccc2c3ccccc3oc21 ; c1ccc(-n2c3ccccc3c3c4[nH]c5c6ccccc6c6ccccc6c5c4ccc32)cc1 ; c1ccc2c(c1)ccc1c2ccc2[nH]c3c(ccc4sc5ccccc5c43)c21 ; c1ccc2cc3cc4c(cc3cc2c1)oc1ccc2c3ccccc3[nH]c2c14 ; c1ccc(-n2c3ccc4ccccc4c3c3ccc4c5cc6ccccc6cc5[nH]c4c32)cc1]

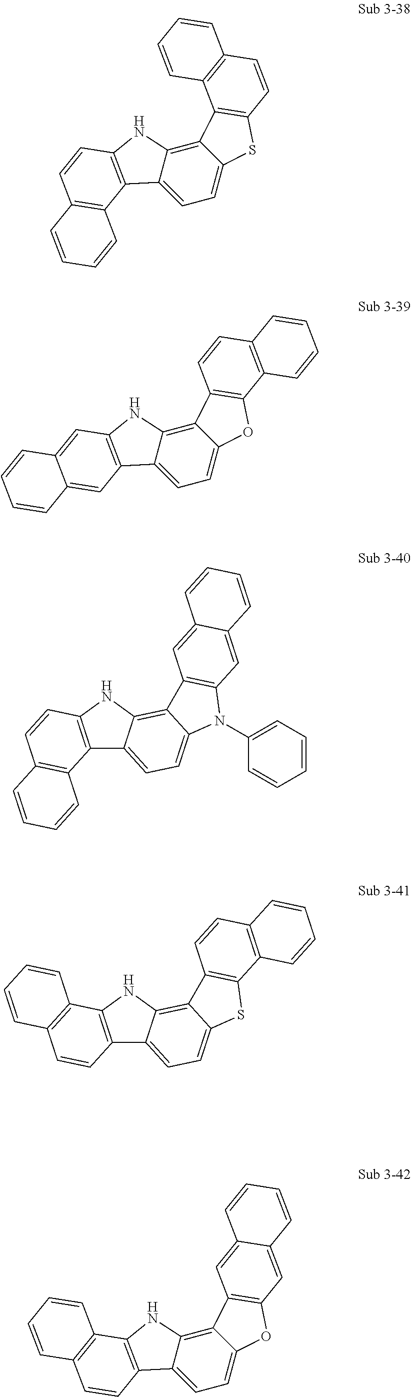 [SMILES: c1ccc2c(c1)ccc1[nH]c3c(ccc4sc5ccc6ccccc6c5c43)c12 ; c1ccc2cc3c(cc2c1)[nH]c1c3ccc2oc3c4ccccc4ccc3c21 ; c1ccc(-n2c3cc4ccccc4cc3c3c4[nH]c5ccc6ccccc6c5c4ccc32)cc1 ; c1ccc2c(c1)ccc1c3ccc4sc5c6ccccc6ccc5c4c3[nH]c21 ; c1ccc2cc3c(cc2c1)oc1ccc2c4ccc5ccccc5c4[nH]c2c13]

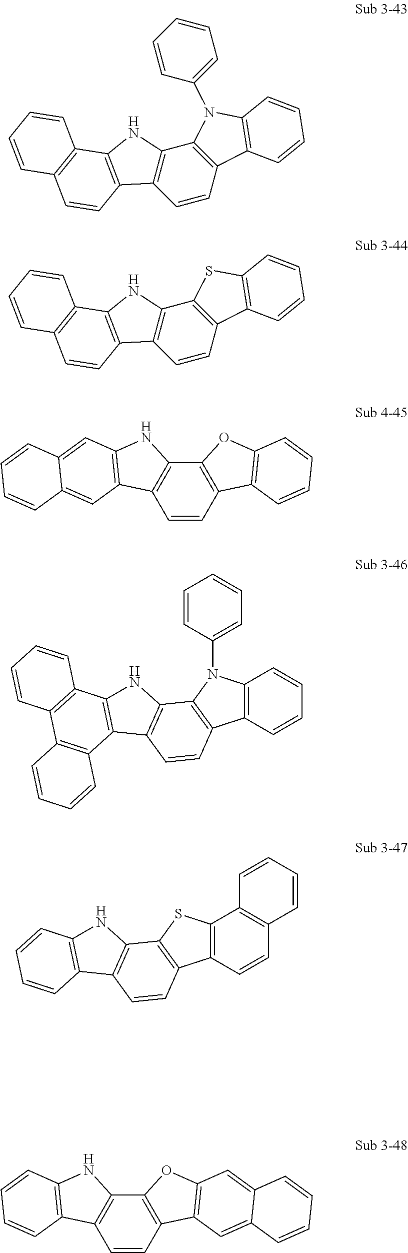 [SMILES: c1ccc(-n2c3ccccc3c3ccc4c5ccc6ccccc6c5[nH]c4c32)cc1 ; c1ccc2c(c1)ccc1c3ccc4c5ccccc5sc4c3[nH]c21 ; c1ccc2cc3c(cc2c1)[nH]c1c3ccc2c3ccccc3oc21 ; c1ccc(-n2c3ccccc3c3ccc4c([nH]c5c6ccccc6c6ccccc6c45)c32)cc1 ; c1ccc2c(c1)ccc1c3ccc4c5ccccc5[nH]c4c3sc21 ; c1ccc2cc3c(cc2c1)oc1c3ccc2c3ccccc3[nH]c21]

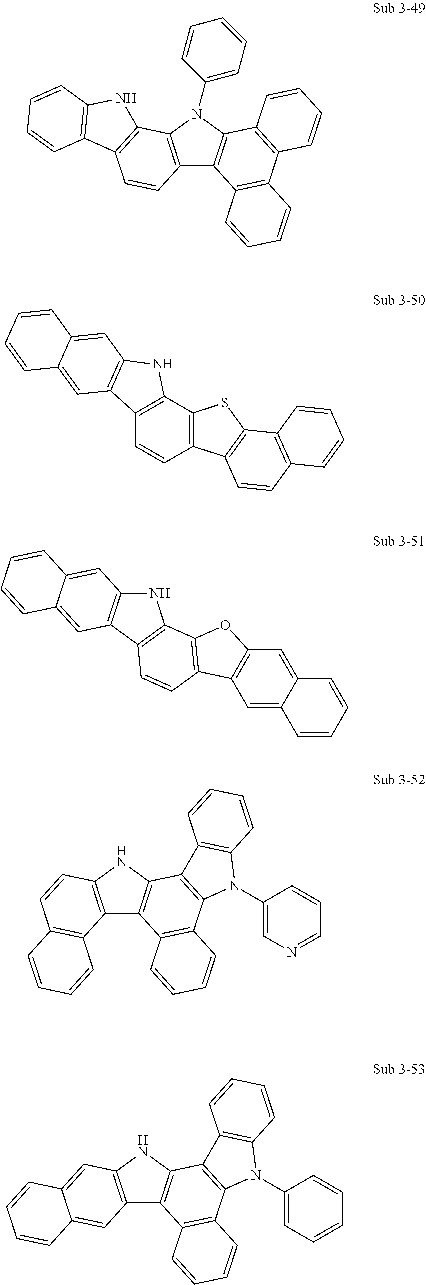 [SMILES: c1ccc(-n2c3c(ccc4c5ccccc5[nH]c43)c3c4ccccc4c4ccccc4c32)cc1 ; c1ccc2cc3c(cc2c1)[nH]c1c3ccc2c3ccc4ccccc4c3sc21 ; c1ccc2cc3c(cc2c1)[nH]c1c3ccc2c3cc4ccccc4cc3oc21 ; c1cncc(-n2c3ccccc3c3c4[nH]c5ccc6ccccc6c5c4c4ccccc4c32)c1 ; c1ccc(-n2c3ccccc3c3c4[nH]c5cc6ccccc6cc5c4c4ccccc4c32)cc1]

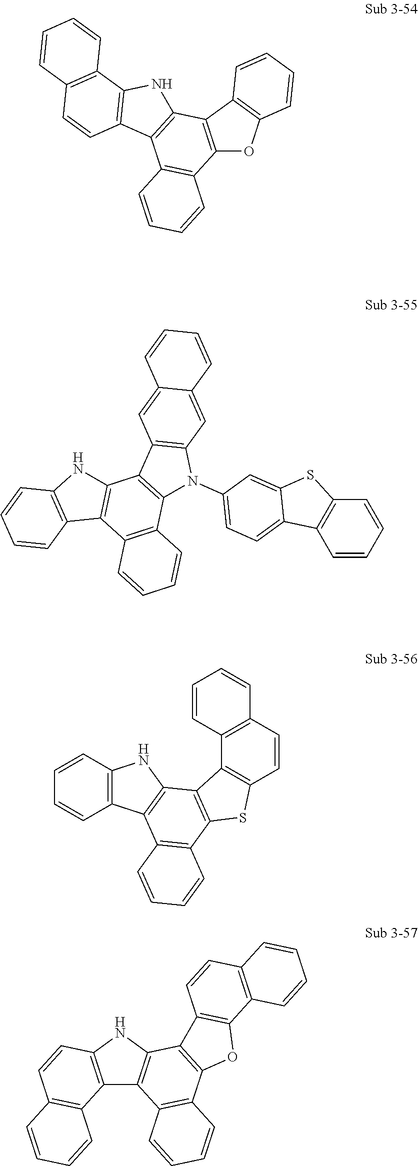 [SMILES: c1ccc2c(c1)ccc1c2[nH]c2c1c1ccccc1c1oc3ccccc3c21 ; c1ccc2cc3c(cc2c1)c1c2[nH]c4ccccc4c2c2ccccc2c1n3-c1ccc2c(c1)sc1ccccc12 ; c1ccc2c(c1)ccc1sc3c4ccccc4c4c5ccccc5[nH]c4c3c12 ; c1ccc2c(c1)ccc1c2oc2c3ccccc3c3c([nH]c4ccc5ccccc5c43)c12]

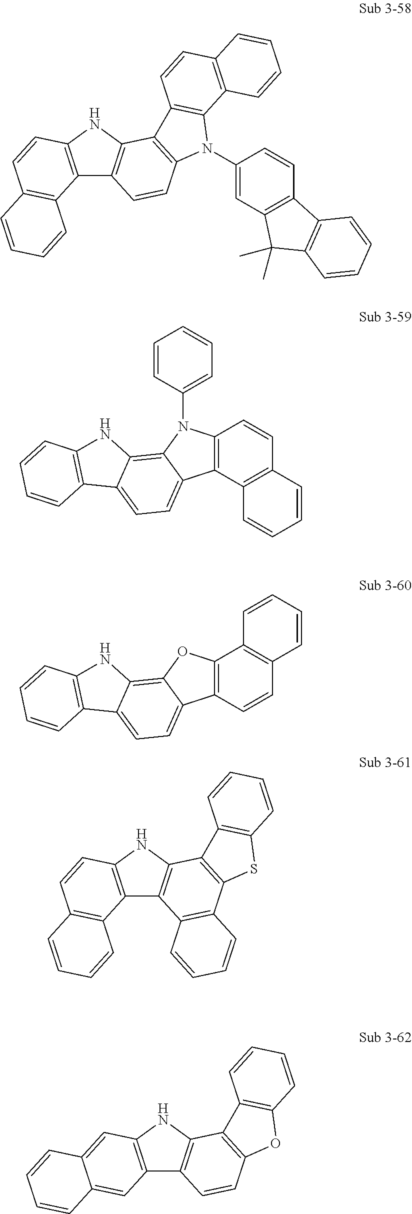 [SMILES: CC1(C)c2ccccc2-c2ccc(-n3c4ccc5c([nH]c6ccc7ccccc7c65)c4c4ccc5ccccc5c43)cc21 ; c1ccc(-n2c3ccc4ccccc4c3c3ccc4c5ccccc5[nH]c4c32)cc1 ; c1ccc2c(c1)ccc1c3ccc4c5ccccc5[nH]c4c3oc21 ; c1ccc2c(c1)ccc1[nH]c3c4c5ccccc5sc4c4ccccc4c3c12 ; c1ccc2cc3c(cc2c1)[nH]c1c3ccc2oc3ccccc3c21]

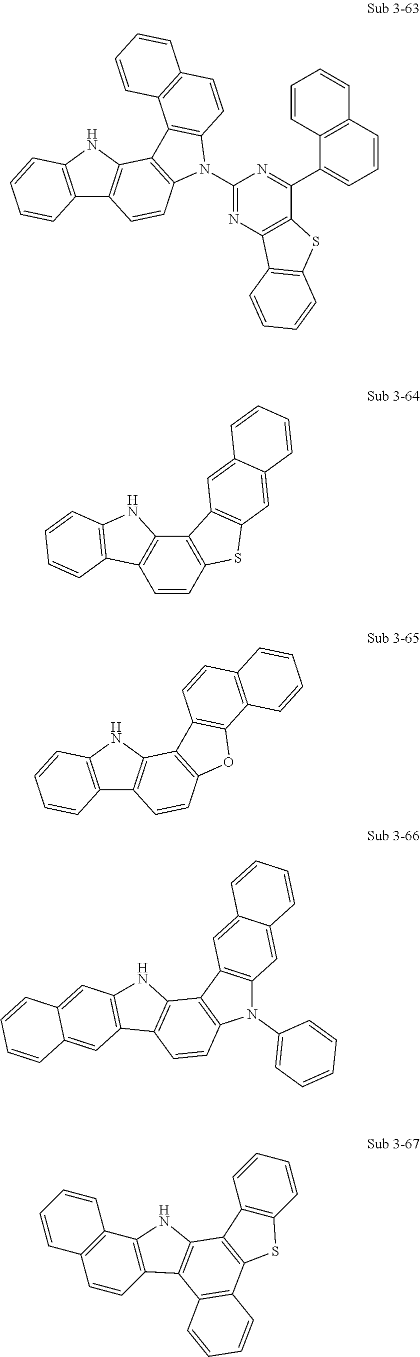 [SMILES: c1ccc2c(-c3nc(-n4c5ccc6ccccc6c5c5c6[nH]c7ccccc7c6ccc54)nc4c3sc3ccccc34)cccc2c1 ; c1ccc2cc3c(cc2c1)sc1ccc2c4ccccc4[nH]c2c13 ; c1ccc2c(c1)ccc1c2oc2ccc3c4ccccc4[nH]c3c21 ; c1ccc(-n2c3cc4ccccc4cc3c3c4[nH]c5cc6ccccc6cc5c4ccc32)cc1 ; c1ccc2c(c1)ccc1c2[nH]c2c1c1ccccc1c1sc3ccccc3c21]

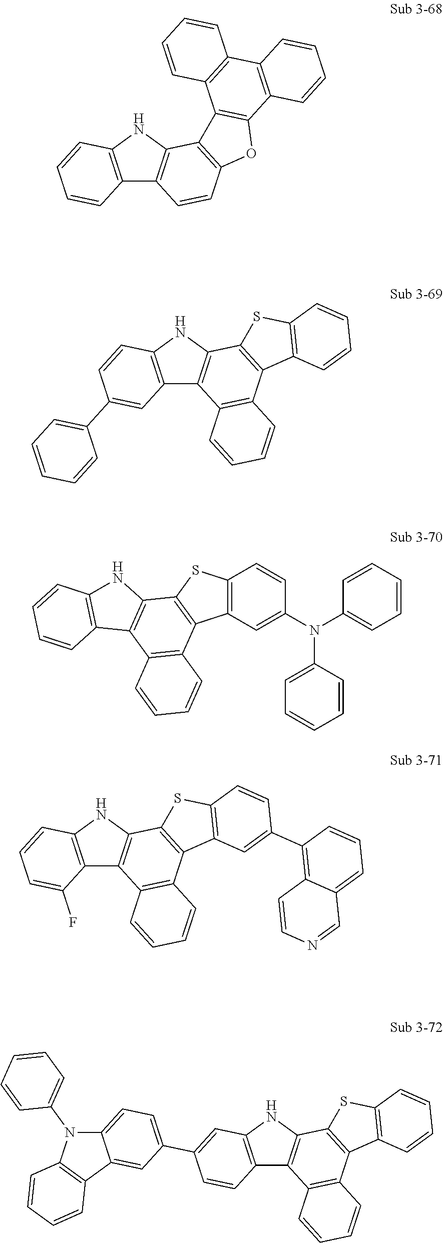 [SMILES: c1ccc2c(c1)[nH]c1c2ccc2oc3c4ccccc4c4ccccc4c3c21 ; c1ccc(-c2ccc3[nH]c4c5sc6ccccc6c5c5ccccc5c4c3c2)cc1 ; c1ccc(N(c2ccccc2)c2ccc3sc4c5[nH]c6ccccc6c5c5ccccc5c4c3c2)cc1 ; Fc1cccc2[nH]c3c4sc5ccc(-c6cccc7cnccc67)cc5c4c4ccccc4c3c12 ; c1ccc(-n2c3ccccc3c3cc(-c4ccc5c(c4)[nH]c4c6sc7ccccc7c6c6ccccc6c54)ccc32)cc1]

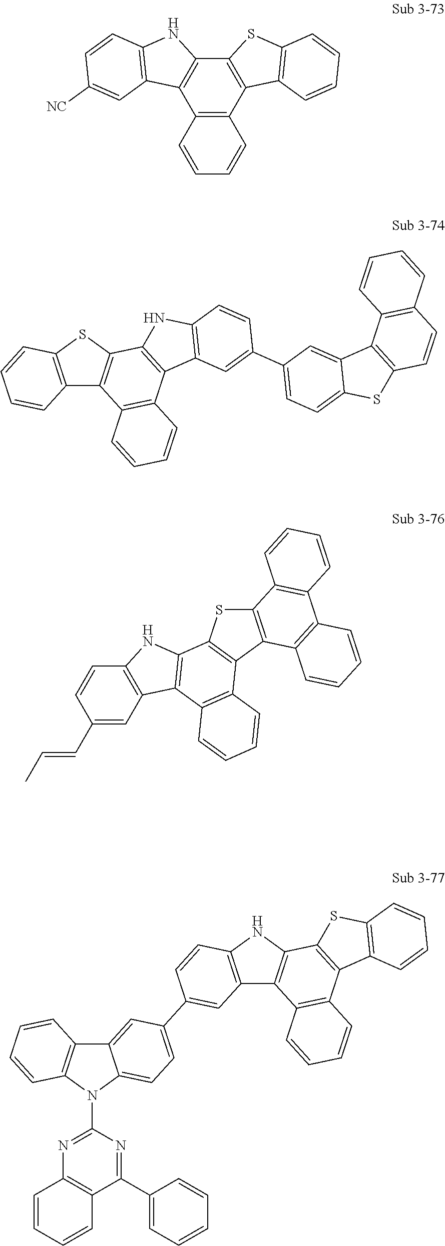 [SMILES: N#Cc1ccc2[nH]c3c4sc5ccccc5c4c4ccccc4c3c2c1 ; c1ccc2c(c1)ccc1sc3ccc(-c4ccc5[nH]c6c7sc8ccccc8c7c7ccccc7c6c5c4)cc3c12 ; C/C=C/c1ccc2[nH]c3c4sc5c6ccccc6c6ccccc6c5c4c4ccccc4c3c2c1 ; c1ccc(-c2nc(-n3c4ccccc4c4cc(-c5ccc6[nH]c7c8sc9ccccc9c8c8ccccc8c7c6c5)ccc43)nc3ccccc23)cc1]

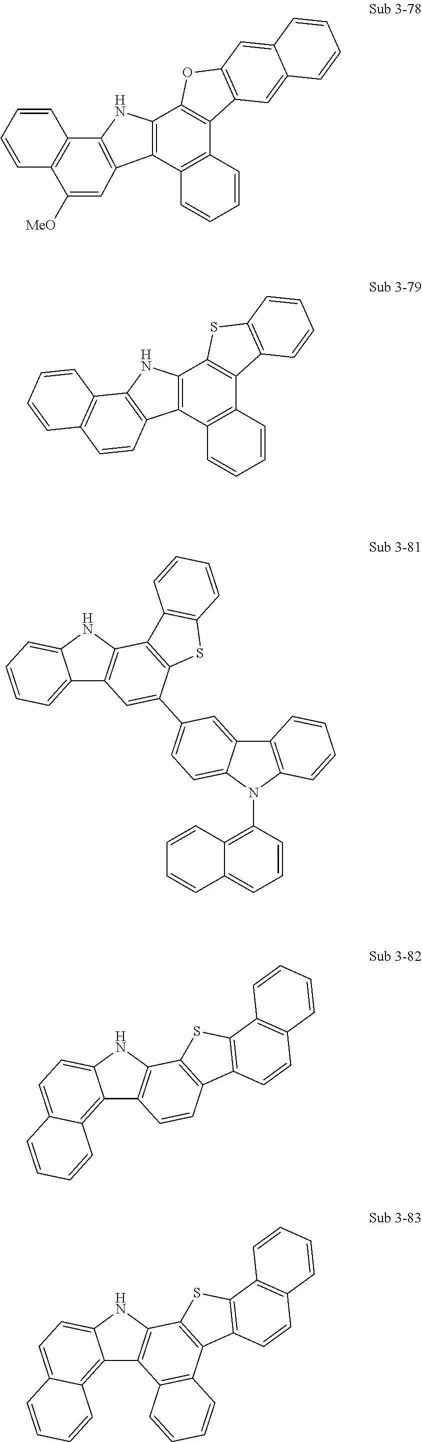 [SMILES: COc1cc2c([nH]c3c4oc5cc6ccccc6cc5c4c4ccccc4c23)c2ccccc12 ; c1ccc2c(c1)ccc1c2[nH]c2c3sc4ccccc4c3c3ccccc3c12 ; c1ccc2c(-n3c4ccccc4c4cc(-c5cc6c7ccccc7[nH]c6c6c5sc5ccccc56)ccc43)cccc2c1 ; c1ccc2c(c1)ccc1c3ccc4c([nH]c5ccc6ccccc6c54)c3sc21 ; c1ccc2c(c1)ccc1c2sc2c3[nH]c4ccc5ccccc5c4c3c3ccccc3c12]

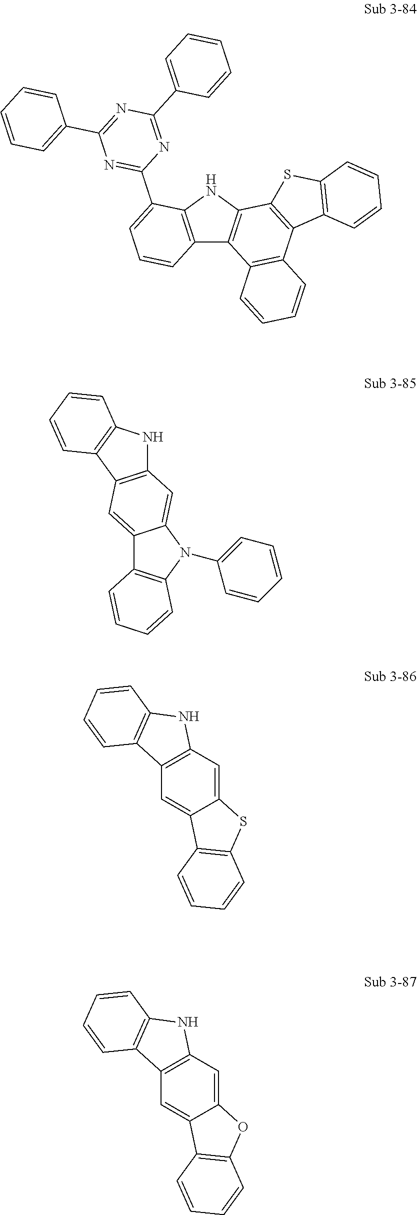 [SMILES: c1ccc(-c2nc(-c3ccccc3)nc(-c3cccc4c3[nH]c3c5sc6ccccc6c5c5ccccc5c43)n2)cc1 ; c1ccc(-n2c3ccccc3c3cc4c(cc32)[nH]c2ccccc24)cc1 ; c1ccc2c(c1)[nH]c1cc3sc4ccccc4c3cc12 ; c1ccc2c(c1)[nH]c1cc3oc4ccccc4c3cc12]

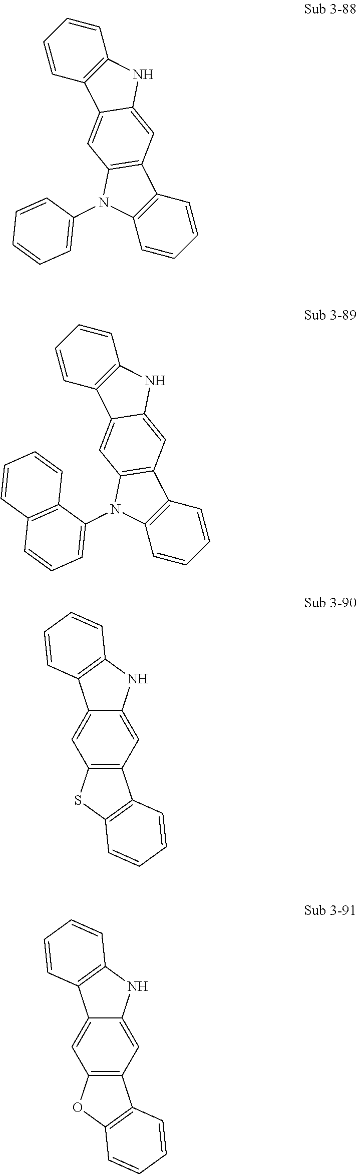

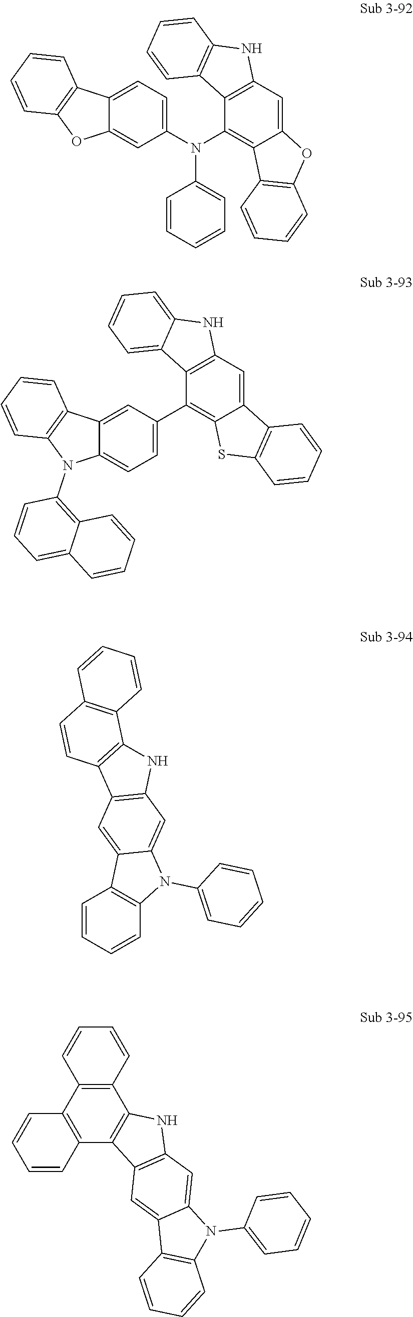 [SMILES: c1ccc(N(c2ccc3c(c2)oc2ccccc23)c2c3c(cc4oc5ccccc5c24)[nH]c2ccccc23)cc1 ; c1ccc2c(-n3c4ccccc4c4cc(-c5c6sc7ccccc7c6cc6[nH]c7ccccc7c56)ccc43)cccc2c1 ; c1ccc(-n2c3ccccc3c3cc4c(cc32)[nH]c2c3ccccc3ccc42)cc1 ; c1ccc(-n2c3ccccc3c3cc4c(cc32)[nH]c2c3ccccc3c3ccccc3c42)cc1]

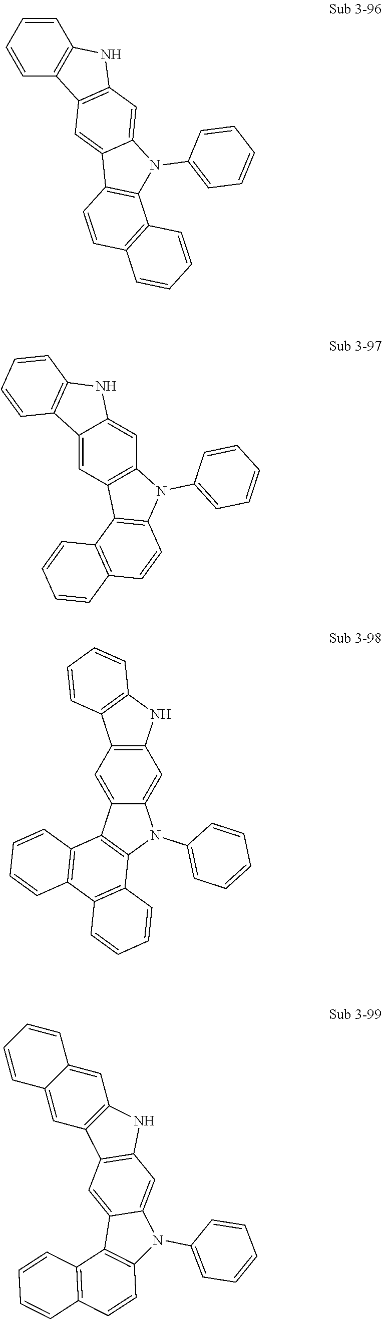

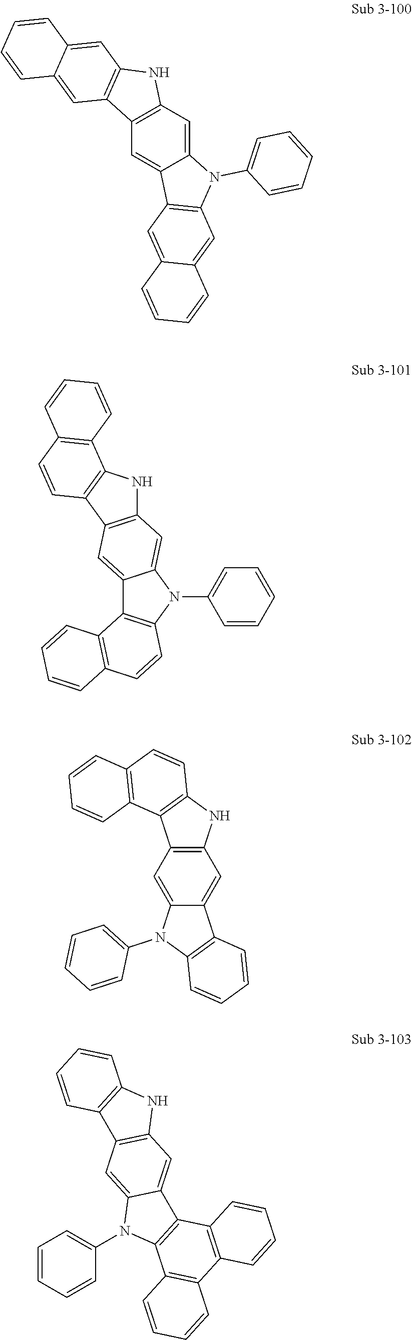 [SMILES: c1ccc(-n2c3cc4ccccc4cc3c3cc4c(cc32)[nH]c2cc3ccccc3cc24)cc1 ; c1ccc(-n2c3cc4[nH]c5c6ccccc6ccc5c4cc3c3c4ccccc4ccc32)cc1 ; c1ccc(-n2c3ccccc3c3cc4[nH]c5ccc6ccccc6c5c4cc32)cc1 ; c1ccc(-n2c3cc4c(cc3c3c5ccccc5c5ccccc5c32)[nH]c2ccccc24)cc1]

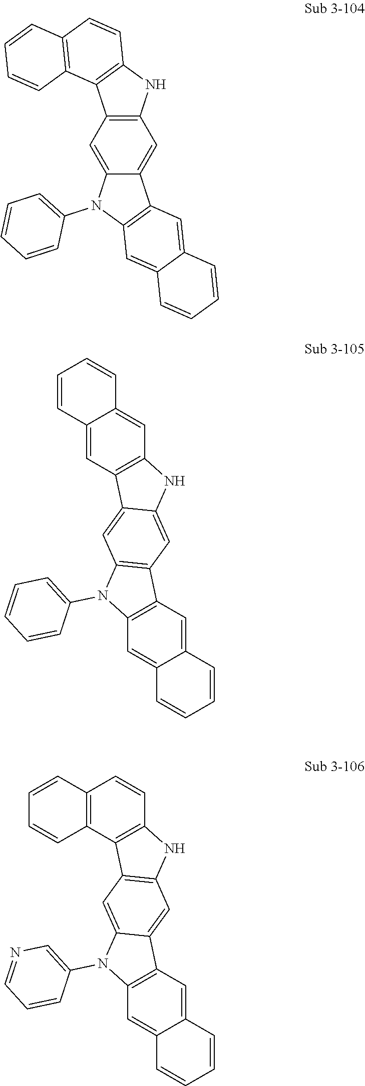 [SMILES: c1ccc(-n2c3cc4ccccc4cc3c3cc4[nH]c5ccc6ccccc6c5c4cc32)cc1 ; c1ccc(-n2c3cc4ccccc4cc3c3cc4[nH]c5cc6ccccc6cc5c4cc32)cc1 ; c1cncc(-n2c3cc4ccccc4cc3c3cc4[nH]c5ccc6ccccc6c5c4cc32)c1]

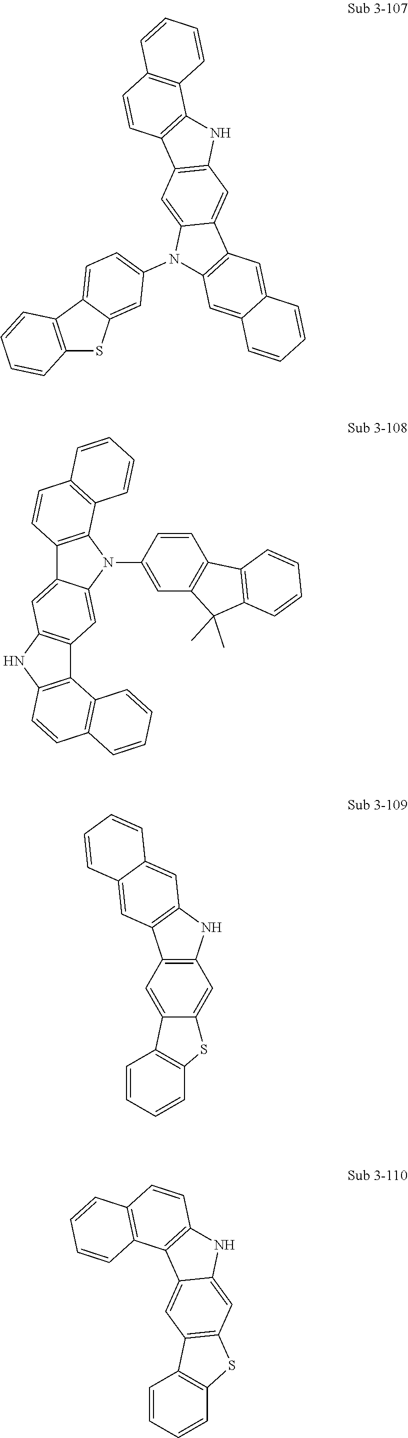 [SMILES: c1ccc2cc3c(cc2c1)c1cc2[nH]c4c5ccccc5ccc4c2cc1n3-c1ccc2c(c1)sc1ccccc12 ; CC1(C)c2ccccc2-c2ccc(-n3c4cc5c(cc4c4ccc6ccccc6c43)[nH]c3ccc4ccccc4c35)cc21 ; c1ccc2cc3c(cc2c1)[nH]c1cc2sc4ccccc4c2cc13 ; c1ccc2c(c1)ccc1[nH]c3cc4sc5ccccc5c4cc3c12]

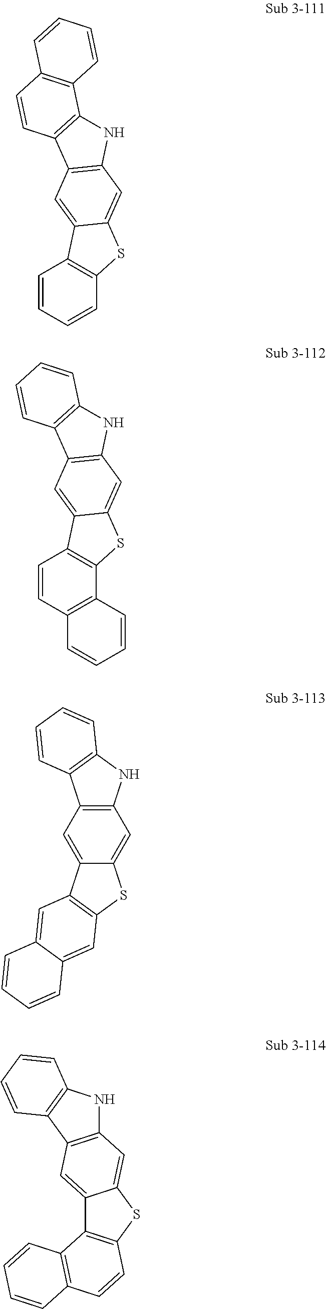

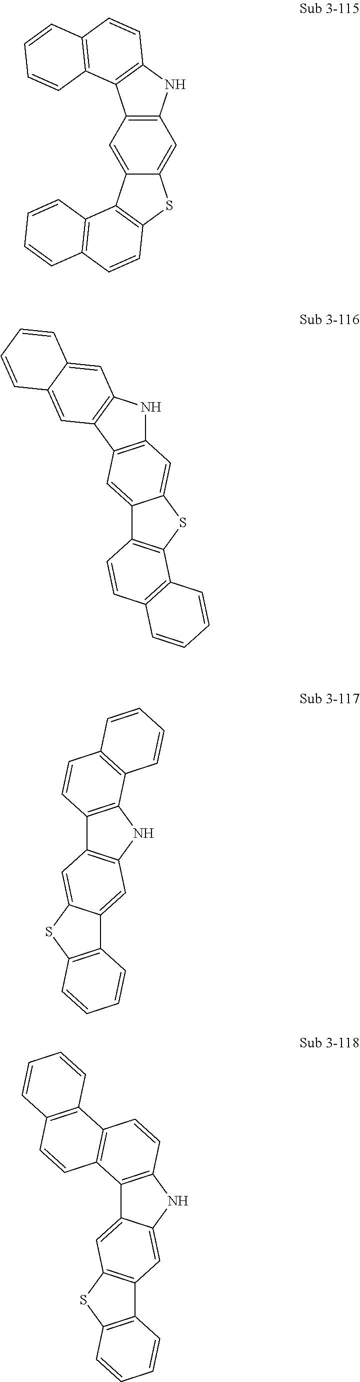

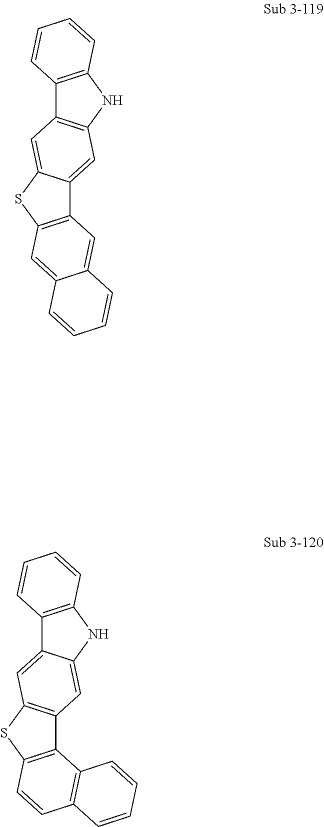 [SMILES: c1ccc2cc3c(cc2c1)sc1cc2c(cc13)[nH]c1ccccc12 ; c1ccc2c(c1)ccc1sc3cc4c(cc3c12)[nH]c1ccccc14]

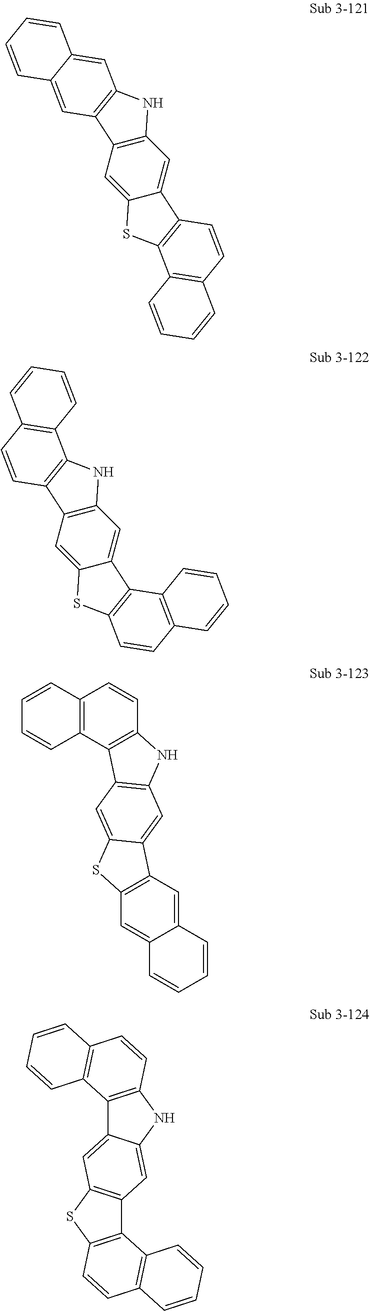

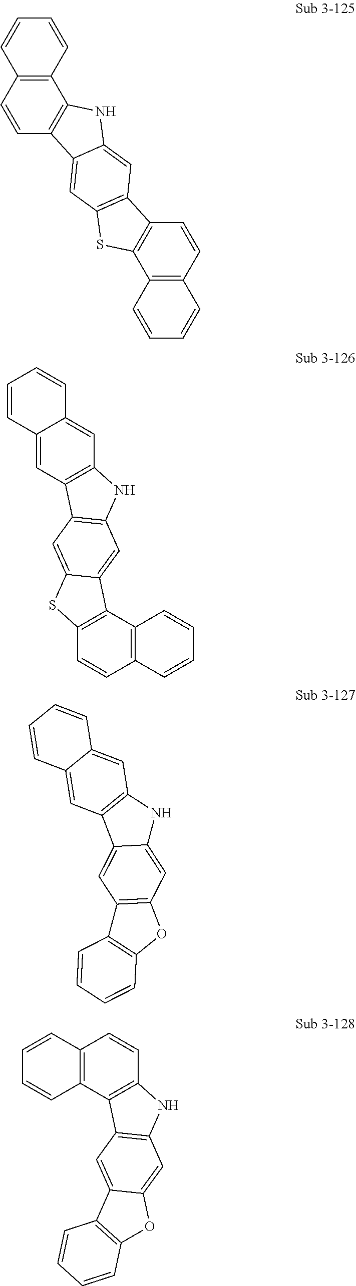

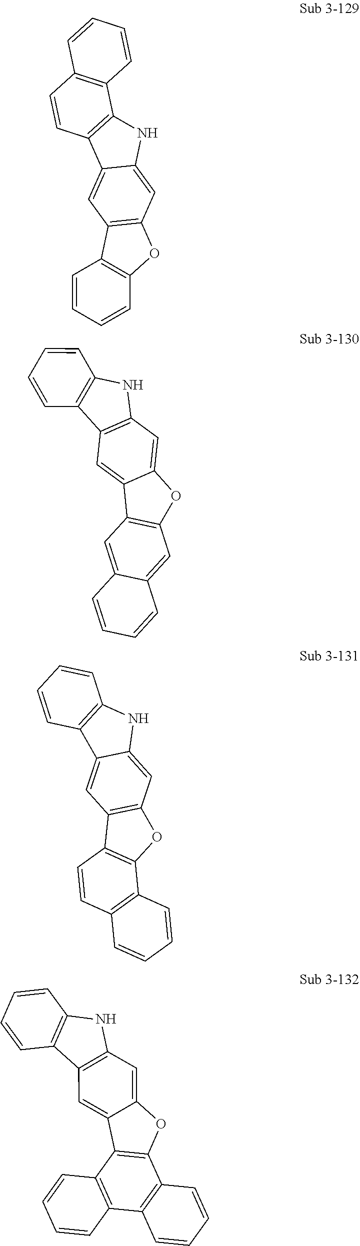

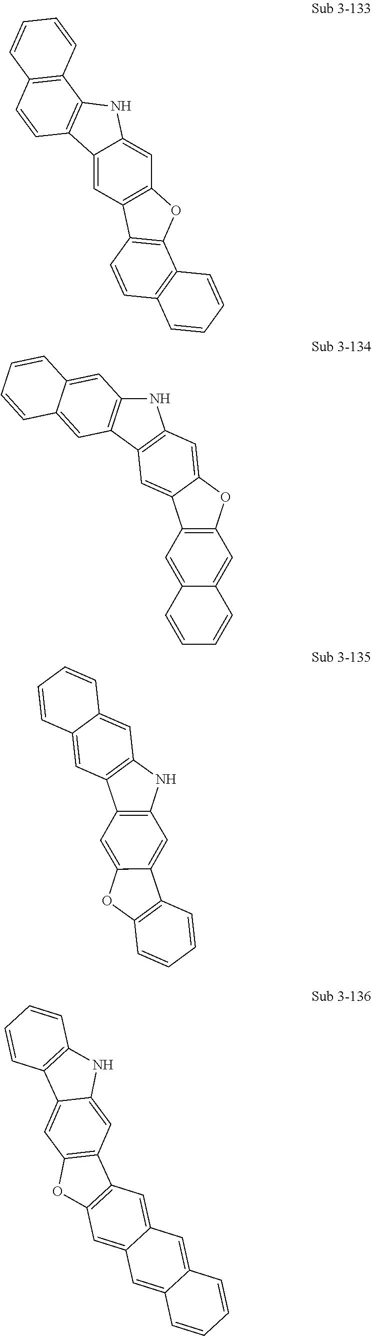 [SMILES: c1ccc2c(c1)ccc1c3cc4c(cc3[nH]c21)oc1c2ccccc2ccc41 ; c1ccc2cc3c(cc2c1)[nH]c1cc2oc4cc5ccccc5cc4c2cc13 ; c1ccc2cc3c(cc2c1)[nH]c1cc2c(cc13)oc1ccccc12 ; c1ccc2cc3cc4c(cc3cc2c1)oc1cc2c(cc14)[nH]c1ccccc12]

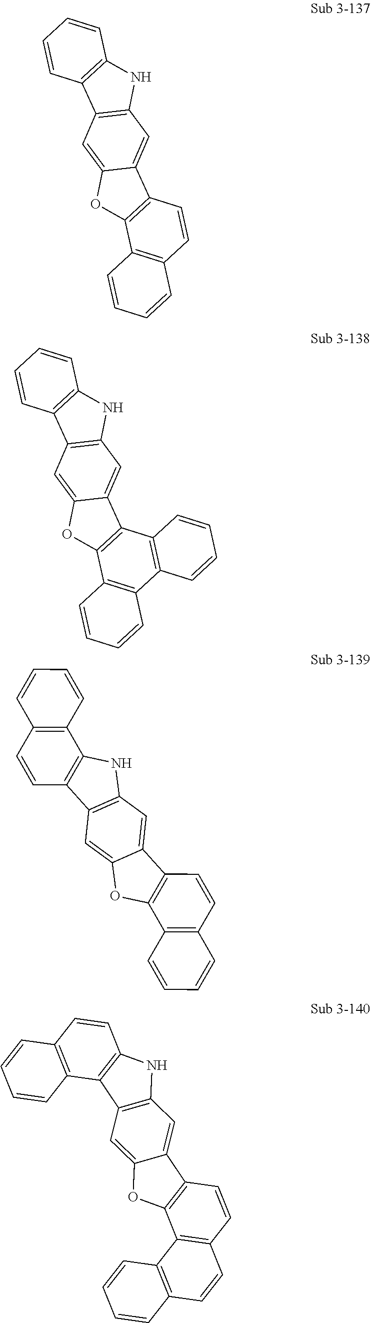

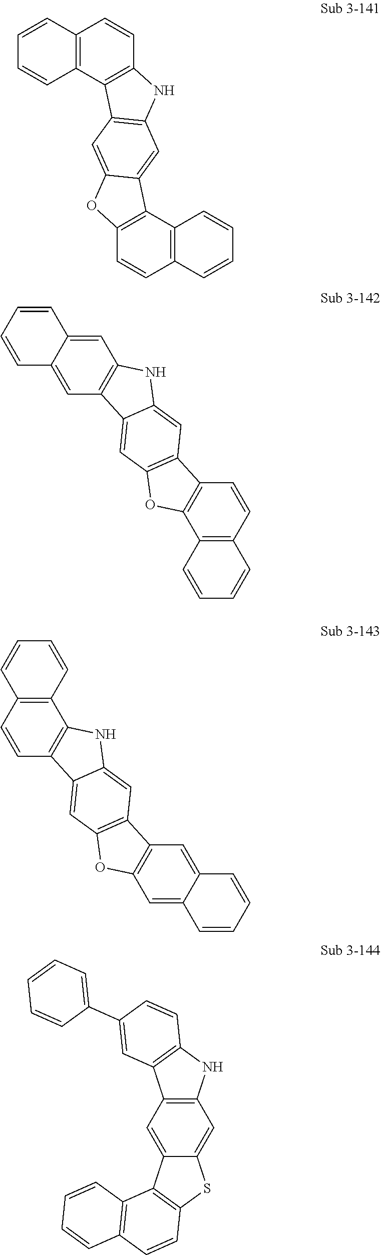 [SMILES: c1ccc2c(c1)ccc1[nH]c3cc4c(cc3c12)oc1ccc2ccccc2c14 ; c1ccc2cc3c(cc2c1)[nH]c1cc2c(cc13)oc1c3ccccc3ccc21 ; c1ccc2cc3c(cc2c1)oc1cc2c(cc13)[nH]c1c3ccccc3ccc21 ; c1ccc(-c2ccc3[nH]c4cc5sc6ccc7ccccc7c6c5cc4c3c2)cc1]

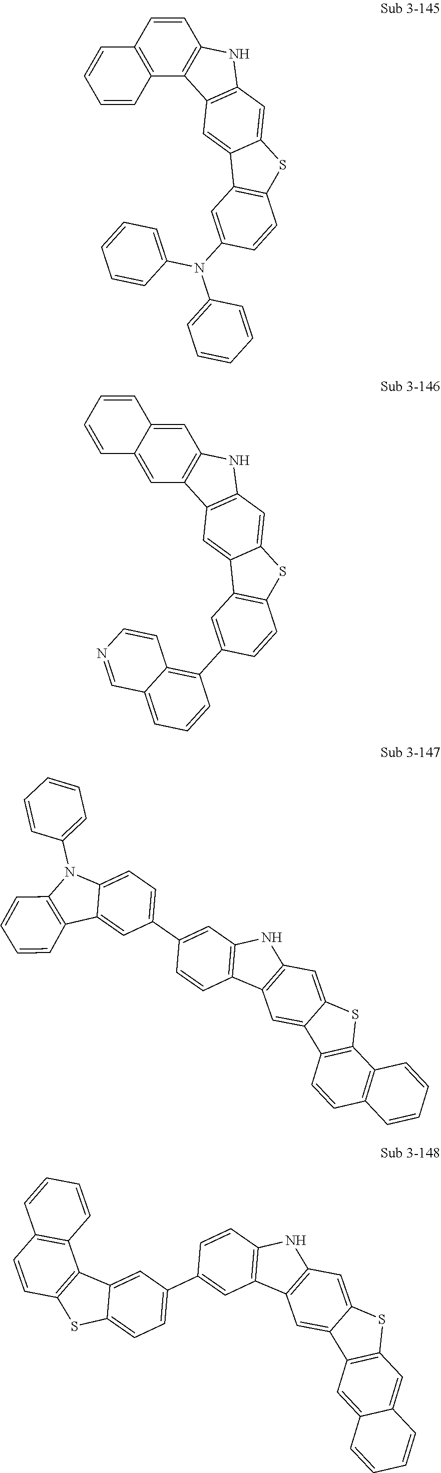 [SMILES: c1ccc(N(c2ccccc2)c2ccc3sc4cc5[nH]c6ccc7ccccc7c6c5cc4c3c2)cc1 ; c1ccc2cc3c(cc2c1)[nH]c1cc2sc4ccc(-c5cccc6cnccc56)cc4c2cc13 ; c1ccc(-n2c3ccccc3c3cc(-c4ccc5c(c4)[nH]c4cc6sc7c8ccccc8ccc7c6cc45)ccc32)cc1 ; c1ccc2cc3c(cc2c1)sc1cc2[nH]c4ccc(-c5ccc6sc7ccc8ccccc8c7c6c5)cc4c2cc13]

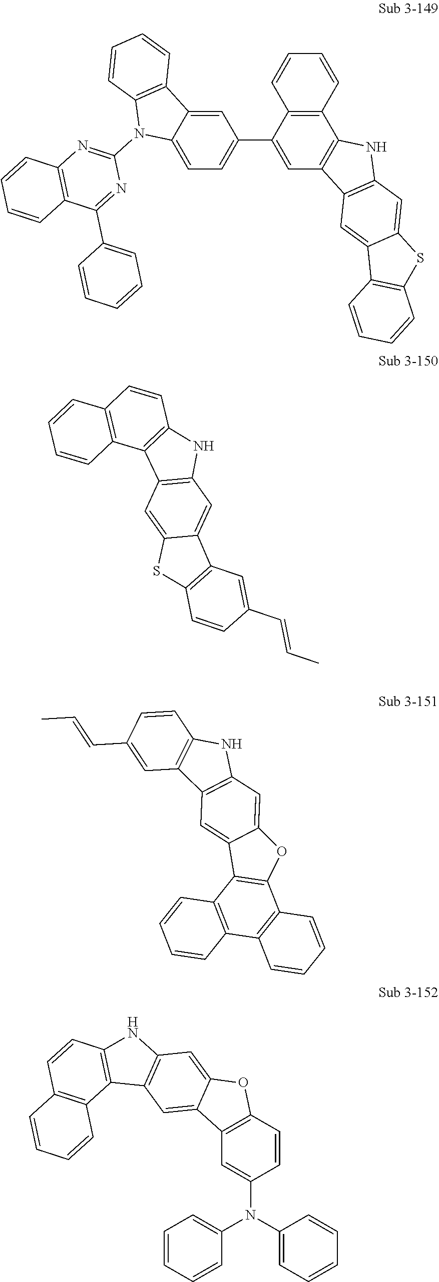

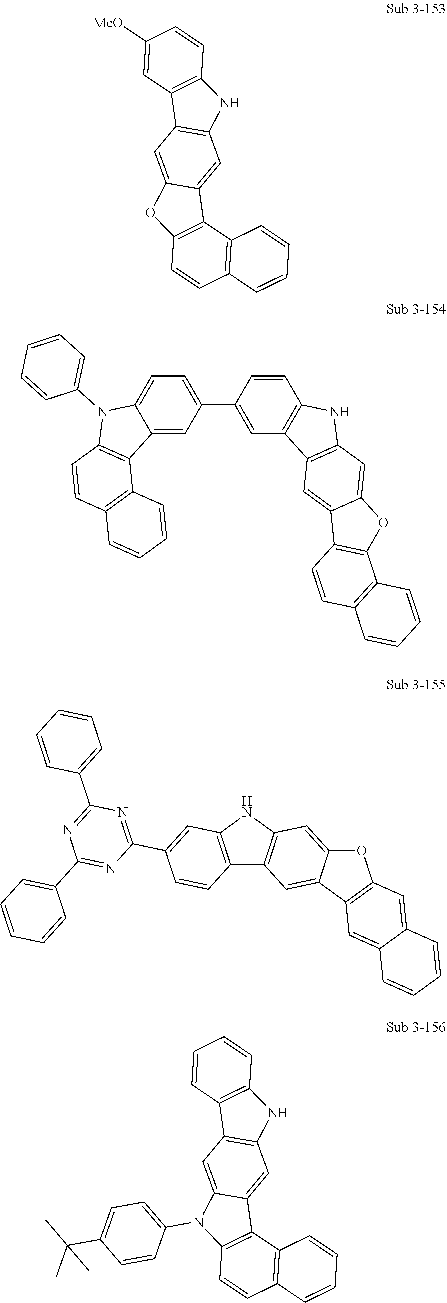 [SMILES: COc1ccc2[nH]c3cc4c(cc3c2c1)oc1ccc2ccccc2c14 ; c1ccc(-n2c3ccc(-c4ccc5[nH]c6cc7oc8c9ccccc9ccc8c7cc6c5c4)cc3c3c4ccccc4ccc32)cc1 ; c1ccc(-c2nc(-c3ccccc3)nc(-c3ccc4c(c3)[nH]c3cc5oc6cc7ccccc7cc6c5cc34)n2)cc1 ; CC(C)(C)c1ccc(-n2c3cc4c(cc3c3c5ccccc5ccc32)[nH]c2ccccc24)cc1]

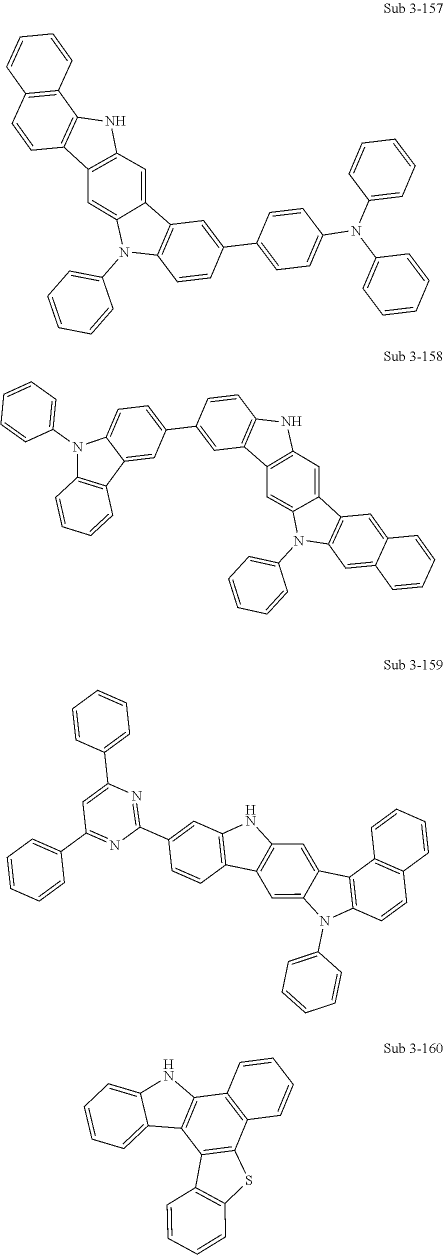 [SMILES: c1ccc(N(c2ccccc2)c2ccc(-c3ccc4c(c3)c3cc5[nH]c6c7ccccc7ccc6c5cc3n4-c3ccccc3)cc2)cc1 ; c1ccc(-n2c3ccccc3c3cc(-c4ccc5[nH]c6cc7c8cc9ccccc9cc8n(-c8ccccc8)c7cc6c5c4)ccc32)cc1 ; c1ccc(-c2cc(-c3ccccc3)nc(-c3ccc4c(c3)[nH]c3cc5c6c7ccccc7ccc6n(-c6ccccc6)c5cc34)n2)cc1 ; c1ccc2c(c1)[nH]c1c3ccccc3c3sc4ccccc4c3c21]

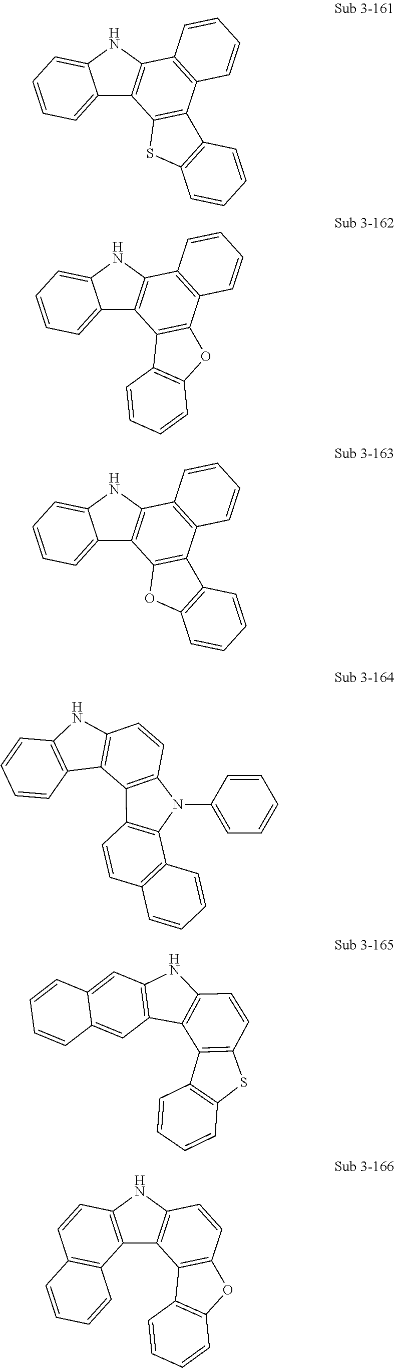

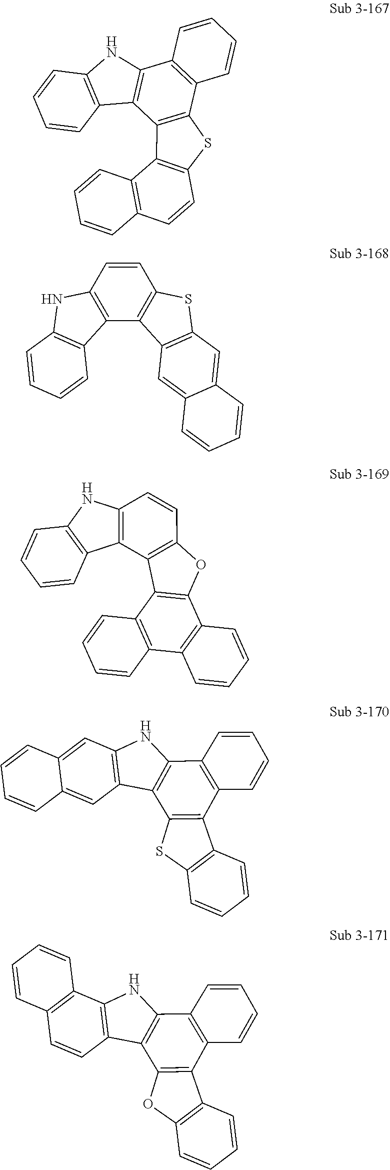

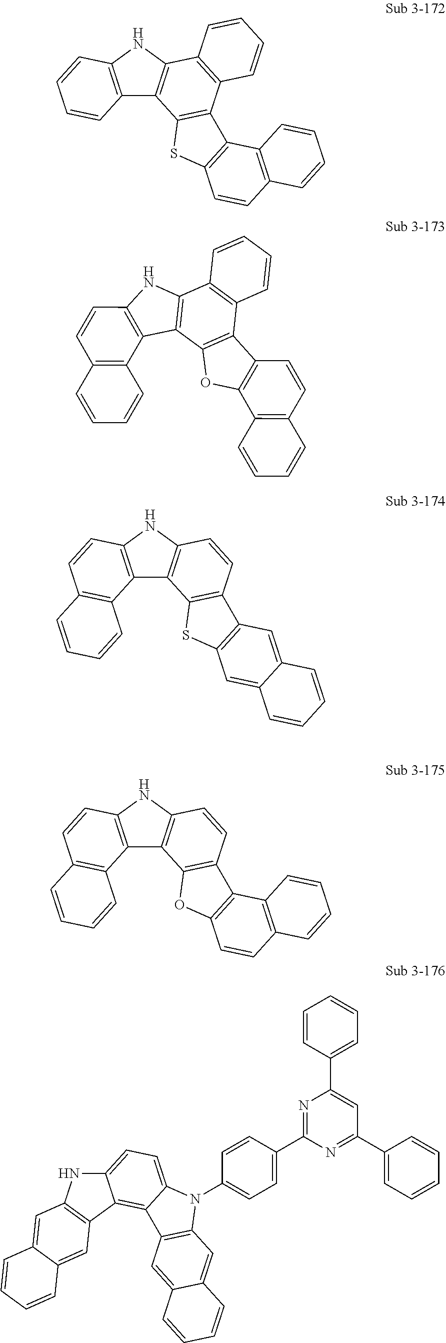

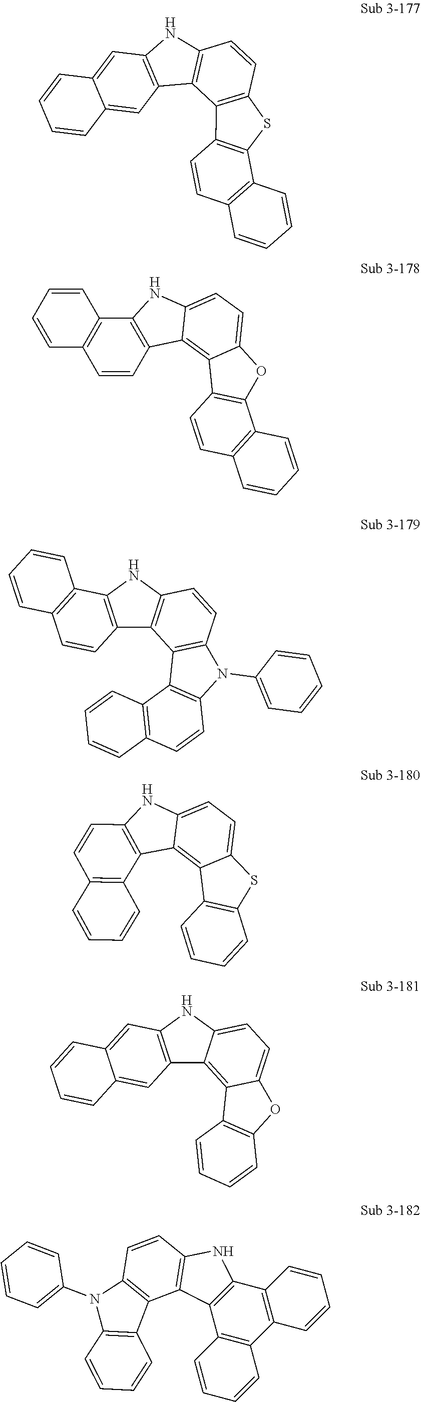 [SMILES: c1ccc2cc3c(cc2c1)[nH]c1ccc2sc4c5ccccc5ccc4c2c13 ; c1ccc2c(c1)ccc1c2[nH]c2ccc3oc4c5ccccc5ccc4c3c21 ; c1ccc(-n2c3ccc4ccccc4c3c3c4c(ccc32)[nH]c2c3ccccc3ccc24)cc1 ; c1ccc2c(c1)ccc1[nH]c3ccc4sc5ccccc5c4c3c12 ; c1ccc2cc3c(cc2c1)[nH]c1ccc2oc4ccccc4c2c13 ; c1ccc(-n2c3ccccc3c3c4c(ccc32)[nH]c2c3ccccc3c3ccccc3c24)cc1]

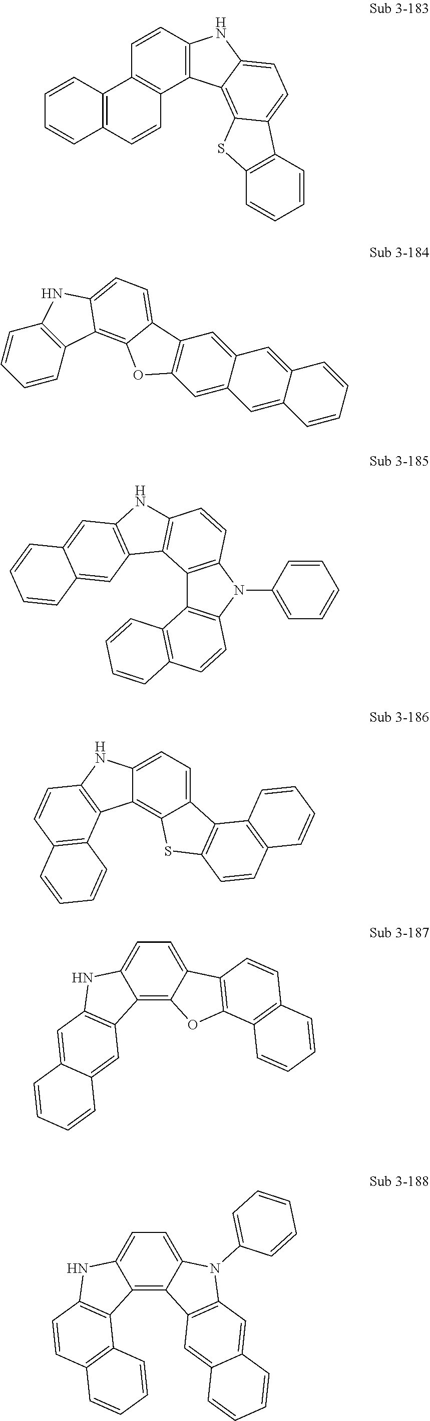 [SMILES: c1ccc2c(c1)ccc1c2ccc2[nH]c3ccc4c5ccccc5sc4c3c21 ; c1ccc2cc3cc4c(cc3cc2c1)oc1c4ccc2[nH]c3ccccc3c21 ; c1ccc(-n2c3ccc4ccccc4c3c3c4c(ccc32)[nH]c2cc3ccccc3cc24)cc1 ; c1ccc2c(c1)ccc1sc3c(ccc4[nH]c5ccc6ccccc6c5c43)c12 ; c1ccc2cc3c(cc2c1)[nH]c1ccc2c4ccc5ccccc5c4oc2c13 ; c1ccc(-n2c3cc4ccccc4cc3c3c4c(ccc32)[nH]c2ccc3ccccc3c24)cc1]

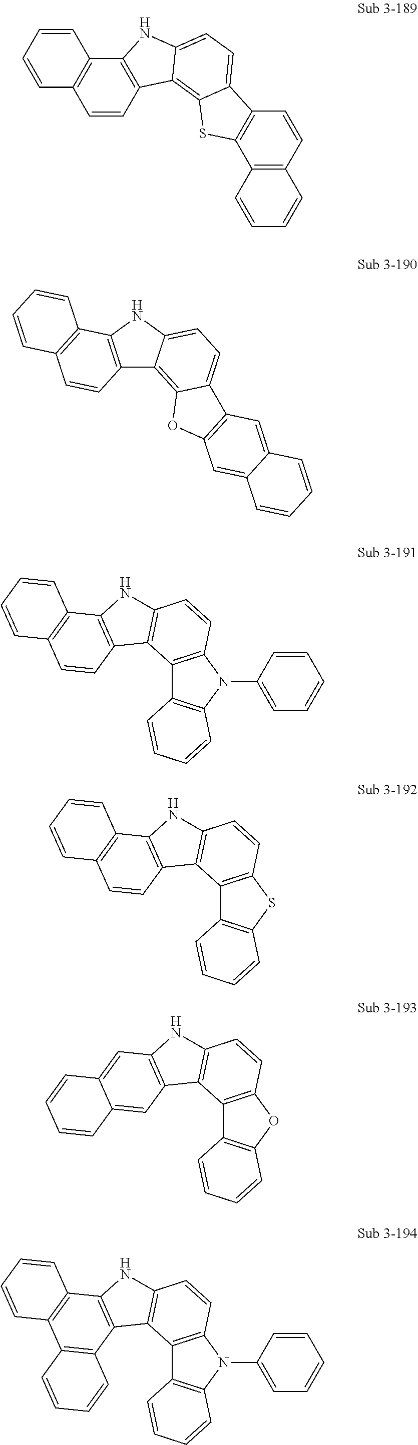 [SMILES: c1ccc2c(c1)ccc1c2[nH]c2ccc3c4ccc5ccccc5c4sc3c21 ; c1ccc2cc3c(cc2c1)oc1c3ccc2[nH]c3c4ccccc4ccc3c21 ; c1ccc(-n2c3ccccc3c3c4c(ccc32)[nH]c2c3ccccc3ccc24)cc1 ; c1ccc2c(c1)ccc1c2[nH]c2ccc3sc4ccccc4c3c21 ; c1ccc2cc3c(cc2c1)[nH]c1ccc2oc4ccccc4c2c13 ; c1ccc(-n2c3ccccc3c3c4c(ccc32)[nH]c2c3ccccc3c3ccccc3c24)cc1]

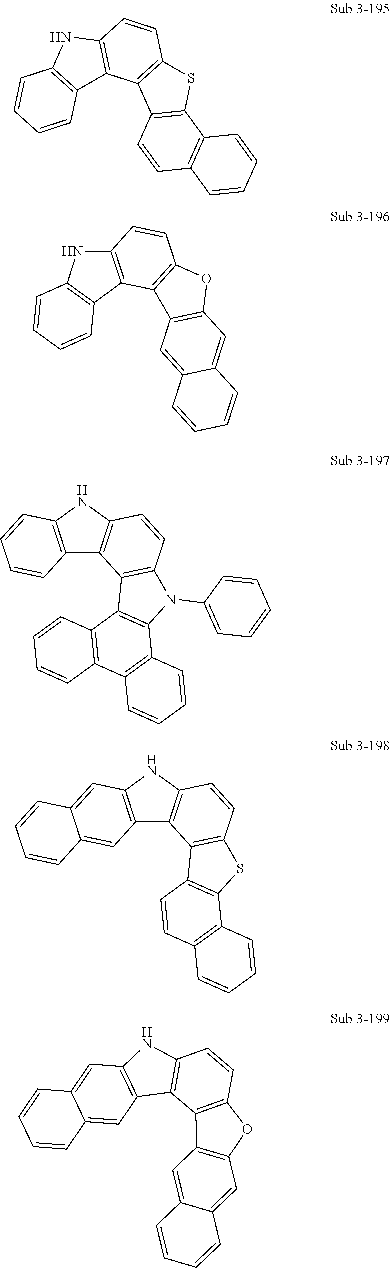 [SMILES: c1ccc2c(c1)ccc1c2sc2ccc3[nH]c4ccccc4c3c21 ; c1ccc2cc3c(cc2c1)oc1ccc2[nH]c4ccccc4c2c13 ; c1ccc(-n2c3ccc4[nH]c5ccccc5c4c3c3c4ccccc4c4ccccc4c32)cc1 ; c1ccc2cc3c(cc2c1)[nH]c1ccc2sc4c5ccccc5ccc4c2c13 ; c1ccc2cc3c(cc2c1)[nH]c1ccc2oc4cc5ccccc5cc4c2c13]

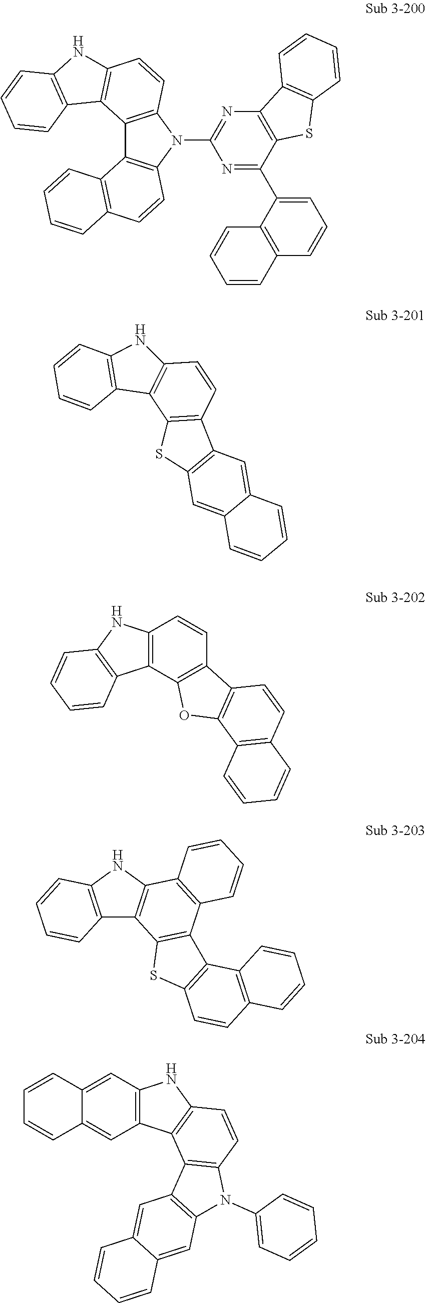

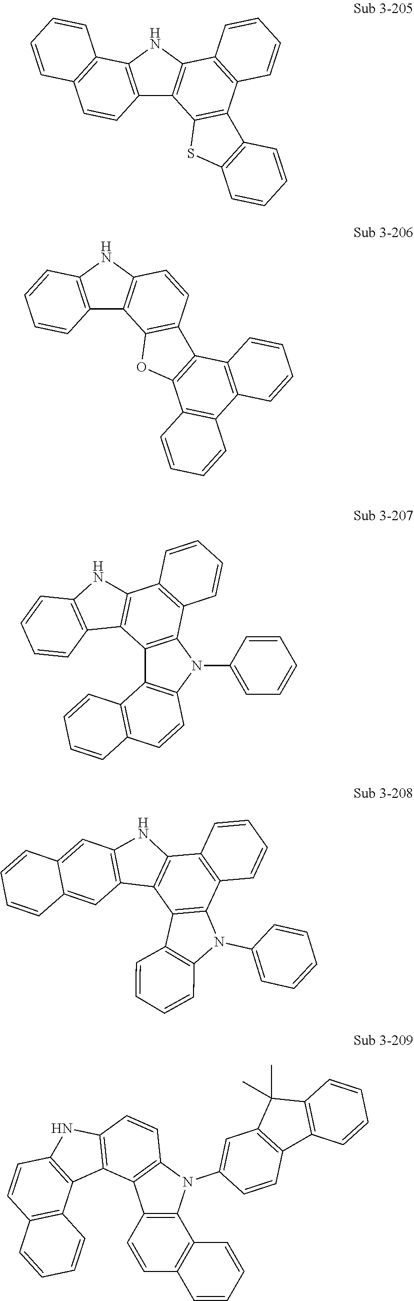 [SMILES: c1ccc2c(c1)ccc1c2[nH]c2c3ccccc3c3c4ccccc4sc3c12 ; c1ccc2c(c1)[nH]c1ccc3c(oc4c5ccccc5c5ccccc5c34)c12 ; c1ccc(-n2c3ccc4ccccc4c3c3c4c5ccccc5[nH]c4c4ccccc4c32)cc1 ; c1ccc(-n2c3ccccc3c3c4c5cc6ccccc6cc5[nH]c4c4ccccc4c32)cc1 ; CC1(C)c2ccccc2-c2ccc(-n3c4ccc5[nH]c6ccc7ccccc7c6c5c4c4ccc5ccccc5c43)cc21]

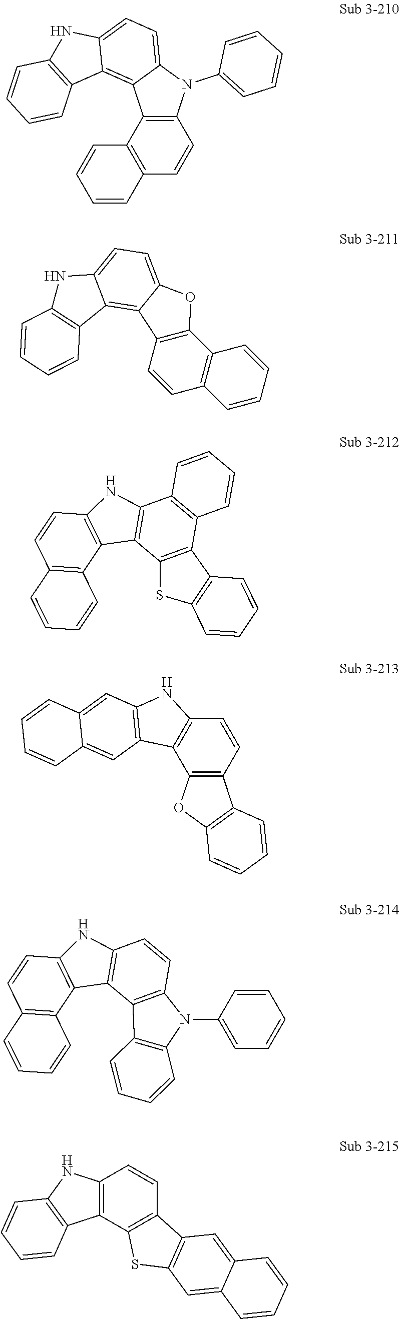

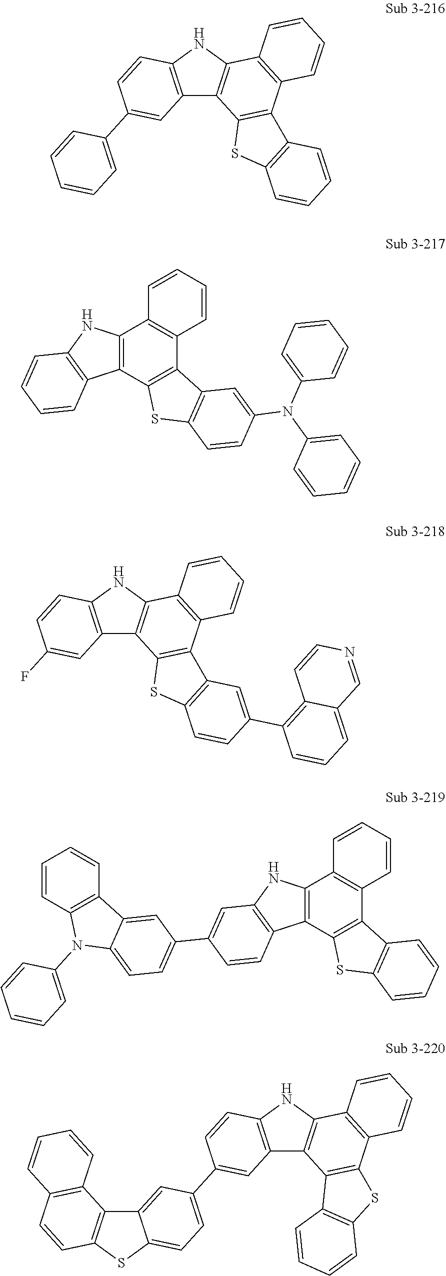 [SMILES: c1ccc(-c2ccc3[nH]c4c5ccccc5c5c6ccccc6sc5c4c3c2)cc1 ; c1ccc(N(c2ccccc2)c2ccc3sc4c(c3c2)c2ccccc2c2[nH]c3ccccc3c24)cc1 ; Fc1ccc2[nH]c3c4ccccc4c4c5cc(-c6cccc7cnccc67)ccc5sc4c3c2c1 ; c1ccc(-n2c3ccccc3c3cc(-c4ccc5c(c4)[nH]c4c6ccccc6c6c7ccccc7sc6c54)ccc32)cc1 ; c1ccc2c(c1)ccc1sc3ccc(-c4ccc5[nH]c6c7ccccc7c7sc8ccccc8c7c6c5c4)cc3c12]

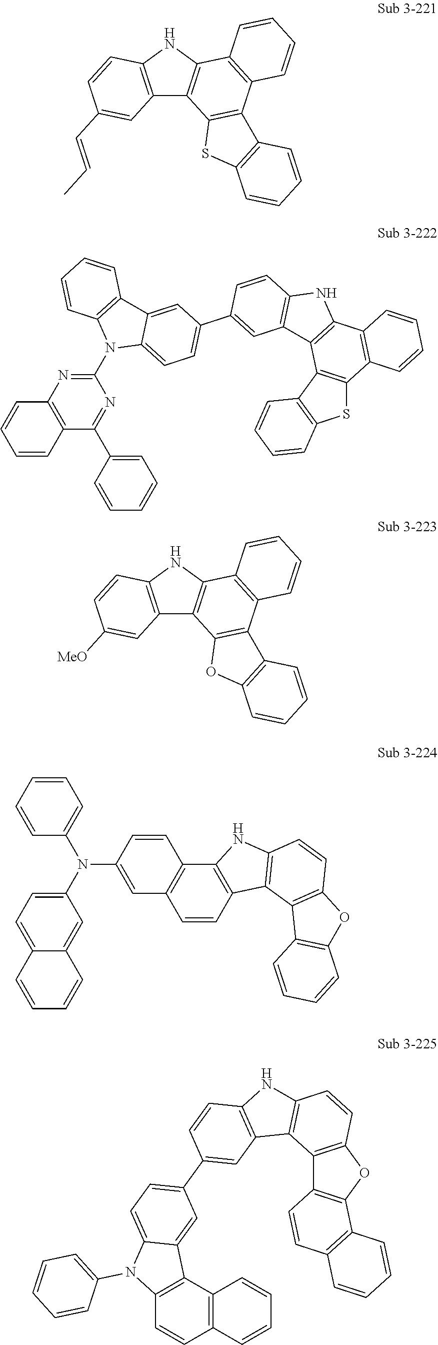 [SMILES: C/C=C/c1ccc2[nH]c3c4ccccc4c4c5ccccc5sc4c3c2c1 ; c1ccc(-c2nc(-n3c4ccccc4c4cc(-c5ccc6[nH]c7c8ccccc8c8sc9ccccc9c8c7c6c5)ccc43)nc3ccccc23)cc1 ; COc1ccc2[nH]c3c4ccccc4c4c5ccccc5oc4c3c2c1 ; c1ccc(N(c2ccc3ccccc3c2)c2ccc3c(ccc4c3[nH]c3ccc5oc6ccccc6c5c34)c2)cc1 ; c1ccc(-n2c3ccc(-c4ccc5[nH]c6ccc7oc8c9ccccc9ccc8c7c6c5c4)cc3c3c4ccccc4ccc32)cc1]

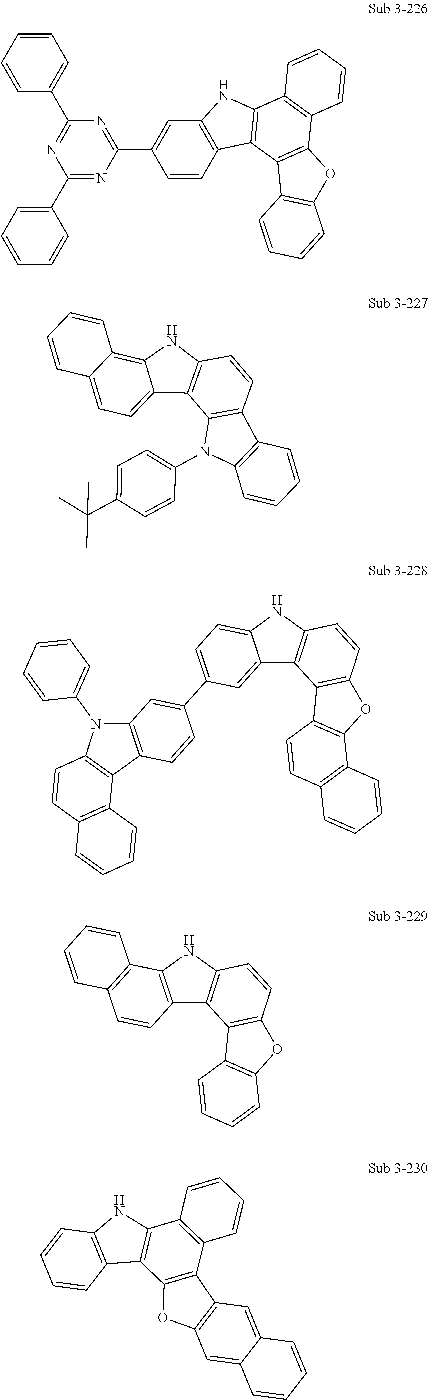 [SMILES: c1ccc(-c2nc(-c3ccccc3)nc(-c3ccc4c(c3)[nH]c3c5ccccc5c5oc6ccccc6c5c43)n2)cc1 ; CC(C)(C)c1ccc(-n2c3ccccc3c3ccc4[nH]c5c6ccccc6ccc5c4c32)cc1 ; c1ccc(-n2c3cc(-c4ccc5[nH]c6ccc7oc8c9ccccc9ccc8c7c6c5c4)ccc3c3c4ccccc4ccc32)cc1 ; c1ccc2c(c1)ccc1c2[nH]c2ccc3oc4ccccc4c3c21 ; c1ccc2cc3c(cc2c1)oc1c3c2ccccc2c2[nH]c3ccccc3c21]

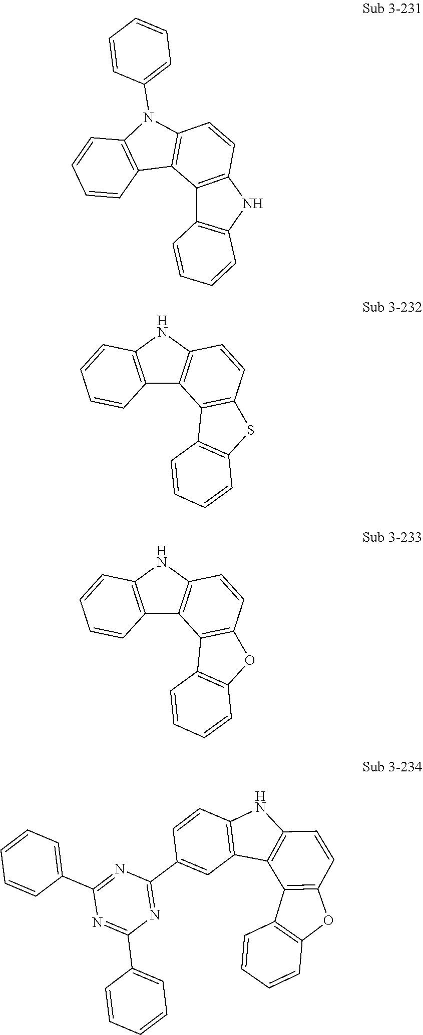 [SMILES: c1ccc(-n2c3ccccc3c3c4c(ccc32)[nH]c2ccccc24)cc1 ; c1ccc2c(c1)[nH]c1ccc3sc4ccccc4c3c12 ; c1ccc2c(c1)[nH]c1ccc3oc4ccccc4c3c12 ; c1ccc(-c2nc(-c3ccccc3)nc(-c3ccc4[nH]c5ccc6oc7ccccc7c6c5c4c3)n2)cc1]

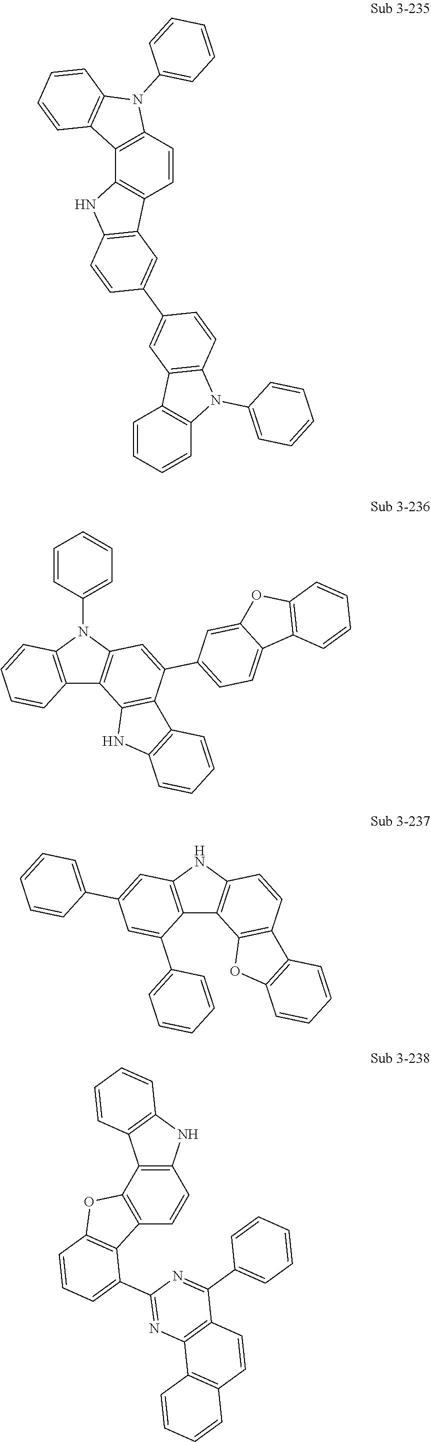 [SMILES: c1ccc(-n2c3ccccc3c3cc(-c4ccc5[nH]c6c(ccc7c6c6ccccc6n7-c6ccccc6)c5c4)ccc32)cc1 ; c1ccc(-n2c3ccccc3c3c4[nH]c5ccccc5c4c(-c4ccc5c(c4)oc4ccccc45)cc32)cc1 ; c1ccc(-c2cc(-c3ccccc3)c3c(c2)[nH]c2ccc4c5ccccc5oc4c23)cc1 ; c1ccc(-c2nc(-c3cccc4oc5c(ccc6[nH]c7ccccc7c65)c34)nc3c2ccc2ccccc23)cc1]

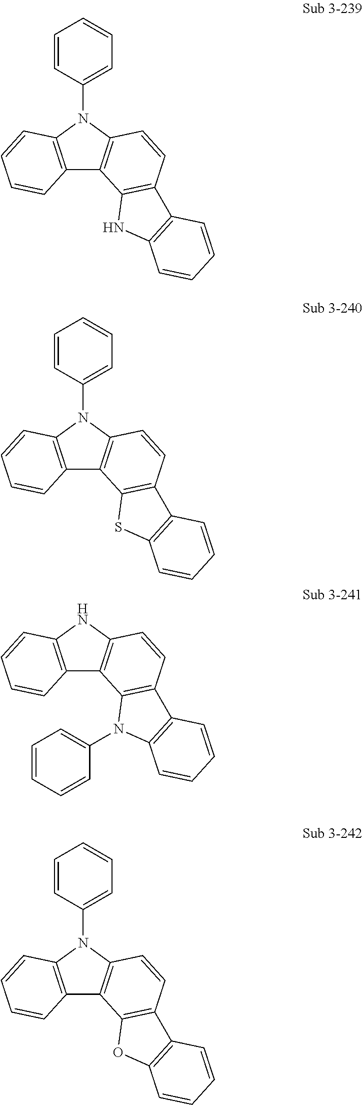 [SMILES: c1ccc(-n2c3ccccc3c3c4[nH]c5ccccc5c4ccc32)cc1 ; c1ccc(-n2c3ccccc3c3c4sc5ccccc5c4ccc32)cc1 ; c1ccc(-n2c3ccccc3c3ccc4[nH]c5ccccc5c4c32)cc1 ; c1ccc(-n2c3ccccc3c3c4oc5ccccc5c4ccc32)cc1]

TABLE 4

| compound | FD-MS |
|---|---|
| Sub 3-1 | m/z = 332.13(C₂₄H₁₆N₂ = 332.41) |
| Sub 3-2 | m/z = 273.06(C₁₈H₁₁NS = 273.35) |
| Sub 3-3 | m/z = 257.08(C₁₈H₁₁NO = 257.29) |
| Sub 3-4 | m/z = 332.13(C₂₄H₁₆N₂ = 332.41) |
| Sub 3-5 | m/z = 382.15(C₂₈H₁₈N₂ = 382.47) |
| Sub 3-6 | m/z = 273.06(C₁₈H₁₁NS = 273.35) |
| Sub 3-7 | m/z = 257.08(C₁₈H₁₁NO = 257.29) |
| Sub 3-8 | m/z = 358.15(C₂₆H₁₈N₂ = 358.44) |
| Sub 3-9 | m/z = 514.17(C₃₆H₂₂N₂O₂ = 514.58) |
| Sub 3-10 | m/z = 357.12(C₂₆H₁₅NO = 357.41) |
| Sub 3-11 | m/z = 323.08(C₂₂H₁₃NS = 323.41) |
| Sub 3-12 | m/z = 323.08(C₂₂H₁₃NS = 323.41) |
| Sub 3-13 | m/z = 307.1(C₂₂H₁₃NO = 307.35) |
| Sub 3-14 | m/z = 307.1(C₂₂H₁₃NO = 307.35) |
| Sub 3-15 | m/z = 382.15(C₂₈H₁₈N₂ = 382.47) |
| Sub 3-16 | m/z = 382.15(C₂₈H₁₈N₂ = 382.47) |
| Sub 3-17 | m/z = 323.08(C₂₂H₁₃NS = 323.41) |
| Sub 3-18 | m/z = 307.1(C₂₂H₁₃NO = 307.35) |
| Sub 3-19 | m/z = 373.09(C₂₆H₁₅NS = 373.47) |
| Sub 3-20 | m/z = 323.08(C₂₂H₁₃NS = 323.41) |
| Sub 3-21 | m/z = 357.12(C₂₆H₁₅NO = 357.41) |
| Sub 3-22 | m/z = 373.09(C₂₆H₁₅NS = 373.47) |
| Sub 3-23 | m/z = 357.12(C₂₆H₁₅NO = 357.41) |
| Sub 3-24 | m/z = 373.09(C₂₆H₁₅NS = 373.47) |
| Sub 3-25 | m/z = 407.13(C₃₀H₁₇NO = 407.47) |
| Sub 3-26 | m/z = 373.09(C₂₆H₁₅NS = 373.47) |
| Sub 3-27 | m/z = 357.12(C₂₆H₁₅NO = 357.41) |
| Sub 3-28 | m/z = 662.25(C₄₈H₃₀N₄ = 662.8) |
| Sub 3-29 | m/z = 373.09(C₂₆H₁₅NS = 373.47) |
| Sub 3-30 | m/z = 357.12(C₂₆H₁₅NO = 357.41) |
| Sub 3-31 | m/z = 432.16(C₃₂H₂₀N₂ = 432.53) |
| Sub 3-32 | m/z = 323.08(C₂₂H₁₃NS = 323.41) |
| Sub 3-33 | m/z = 307.1(C₂₂H₁₃NO = 307.35) |
| Sub 3-34 | m/z = 432.16(C₃₂H₂₀N₂ = 432.53) |
| Sub 3-35 | m/z = 373.09(C₂₆H₁₅NS = 373.47) |
| Sub 3-36 | m/z = 357.12(C₂₆H₁₅NO = 357.41) |
| Sub 3-37 | m/z = 432.16(C₃₂H₂₀N₂ = 432.53) |
| Sub 3-38 | m/z = 373.09(C₂₆H₁₅NS = 373.47) |
| Sub 3-39 | m/z = 357.12(C₂₆H₁₅NO = 357.41) |
| Sub 3-40 | m/z = 432.16(C₃₂H₂₀N₂ = 432.53) |
| Sub 3-41 | m/z = 373.09(C₂₆H₁₅NS = 373.47) |
| Sub 3-42 | m/z = 357.12(C₂₆H₁₅NO = 357.41) |
| Sub 3-43 | m/z = 382.15(C₂₈H₁₈N₂ = 382.47) |
| Sub 3-44 | m/z = 323.08(C₂₂H₁₃NS = 323.41) |
| Sub 3-45 | m/z = 307.1(C₂₂H₁₃NO = 307.35) |
| Sub 3-46 | m/z = 432.16(C₃₂H₂₀N₂ = 432.53) |
| Sub 3-47 | m/z = 323.08(C₂₂H₁₃NS = 323.41) |
| Sub 3-48 | m/z = 307.1(C₂₂H₁₃NO = 307.35) |
| Sub 3-49 | m/z = 432.16(C₃₂H₂₀N₂ = 432.53) |
| Sub 3-50 | m/z = 373.09(C₂₆H₁₅NS = 373.47) |
| Sub 3-51 | m/z = 357.12(C₂₆H₁₅NO = 357.41) |
| Sub 3-52 | m/z = 433.16(C₃₁H₁₉N₃ = 433.51) |
| Sub 3-53 | m/z = 432.16(C₃₂H₂₀N₂ = 432.53) |
| Sub 3-54 | m/z = 357.12(C₂₆H₁₅NO = 357.41) |
| Sub 3-55 | m/z = 538.15(C₃₈H₂₂N₂S = 538.67) |
| Sub 3-56 | m/z = 373.09(C₂₆H₁₅NS = 373.47) |
| Sub 3-57 | m/z = 407.13(C₃₀H₁₇NO = 407.47) |
| Sub 3-58 | m/z = 548.23(C₄₁H₂₈N₂ = 548.69) |
| Sub 3-59 | m/z = 382.15(C₂₈H₁₈N₂ = 382.47) |
| Sub 3-60 | m/z = 307.1(C₂₂H₁₃NO = 307.35) |
| Sub 3-61 | m/z = 373.09(C₂₆H₁₅NS = 373.47) |
| Sub 3-62 | m/z = 307.1(C₂₂H₁₃NO = 307.35) |
| Sub 3-63 | m/z = 616.17(C₄₂H₂₄N₄S = 616.74) |
| Sub 3-64 | m/z = 323.08(C₂₂H₁₃NS = 323.41) |
| Sub 3-65 | m/z = 307.1(C₂₂H₁₃NO = 307.35) |
| Sub 3-66 | m/z = 432.16(C₃₂H₂₀N₂ = 432.53) |
| Sub 3-67 | m/z = 373.09(C₂₆H₁₅NS = 373.47) |
| Sub 3-68 | m/z = 357.12(C₂₆H₁₅NO = 357.41) |
| Sub 3-69 | m/z = 399.11(C₂₈H₁₇NS = 399.51) |
| Sub 3-70 | m/z = 490.15(C₃₄H₂₂N₂S = 490.62) |
| Sub 3-71 | m/z = 468.11(C₃₁H₁₇FN₂S = 468.55) |
| Sub 3-72 | m/z = 564.17(C₄₀H₂₄N₂S = 564.71) |
| Sub 3-73 | m/z = 348.07(C₂₃H₁₂N₂S = 348.42) |
| Sub 3-74 | m/z = 555.11(C₃₈H₂₁NS₂ = 555.71) |
| Sub 3-76 | m/z = 463.14(C₃₃H₂₁NS = 463.6) |
| Sub 3-77 | m/z = 692.2(C₄₈H₂₈N₄S = 692.84) |
| Sub 3-78 | m/z = 437.14(C₃₁H₁₉NO₂ = 437.5) |
| Sub 3-79 | m/z = 373.09(C₂₆H₁₅NS = 373.47) |
| Sub 3-81 | m/z = 564.17(C₄₀H₂₄N₂S = 564.71) |
| Sub 3-82 | m/z = 373.09(C₂₆H₁₅NS = 373.47) |
| Sub 3-83 | m/z = 423.11(C₃₀H₁₇NS = 423.53) |
| Sub 3-84 | m/z = 554.16(C₃₇H₂₂N₄S = 554.67) |
| Sub 3-85 | m/z = 332.13(C₂₄H₁₆N₂ = 332.41) |
| Sub 3-86 | m/z = 273.06(C₁₈H₁₁NS = 273.35) |
| Sub 3-87 | m/z = 257.08(C₁₈H₁₁NO = 257.29) |
| Sub 3-88 | m/z = 332.13(C₂₄H₁₆N₂ = 332.41) |
| Sub 3-89 | m/z = 382.15(C₂₈H₁₈N₂ = 382.47) |
| Sub 3-90 | m/z = 273.06(C₁₈H₁₁NS = 273.35) |
| Sub 3-91 | m/z = 257.08(C₁₈H₁₁NO = 257.29) |
| Sub 3-92 | m/z = 514.17(C₃₆H₂₂N₂O₂ = 514.58) |
| Sub 3-93 | m/z = 564.17(C₄₀H₂₄N₂S = 564.71) |
| Sub 3-94 | m/z = 382.15(C₂₈H₁₈N₂ = 382.47) |
| Sub 3-95 | m/z = 432.16(C₃₂H₂₀N₂ = 432.53) |
| Sub 3-96 | m/z = 382.15(C₂₈H₁₈N₂ = 382.47) |
| Sub 3-97 | m/z = 382.15(C₂₈H₁₈N₂ = 382.47) |
| Sub 3-98 | m/z = 432.16(C₃₂H₂₀N₂ = 432.53) |
| Sub 3-99 | m/z = 432.16(C₃₂H₂₀N₂ = 432.53) |
| Sub 3-100 | m/z = 432.16(C₃₂H₂₀N₂ = 432.53) |
| Sub 3-101 | m/z = 432.16(C₃₂H₂₀N₂ = 432.53) |
| Sub 3-102 | m/z = 382.15(C₂₈H₁₈N₂ = 382.47) |
| Sub 3-103 | m/z = 432.16(C₃₂H₂₀N₂ = 432.53) |
| Sub 3-104 | m/z = 432.16(C₃₂H₂₀N₂ = 432.53) |
| Sub 3-105 | m/z = 432.16(C₃₂H₂₀N₂ = 432.53) |
| Sub 3-106 | m/z = 433.16(C₃₁H₁₉N₃ = 433.51) |
| Sub 3-107 | m/z = 538.15(C₃₈H₂₂N₂S = 538.67) |
| Sub 3-108 | m/z = 548.23(C₄₁H₂₈N₂ = 548.69) |
| Sub 3-109 | m/z = 323.08(C₂₂H₁₃NS = 323.41) |
| Sub 3-110 | m/z = 323.08(C₂₂H₁₃NS = 323.41) |
| Sub 3-111 | m/z = 323.08(C₂₂H₁₃NS = 323.41) |
| Sub 3-112 | m/z = 323.08(C₂₂H₁₃NS = 323.41) |
| Sub 3-113 | m/z = 323.08(C₂₂H₁₃NS = 323.41) |
| Sub 3-114 | m/z = 323.08(C₂₂H₁₃NS = 323.41) |
| Sub 3-115 | m/z = 373.09(C₂₆H₁₅NS = 373.47) |
| Sub 3-116 | m/z = 373.09(C₂₆H₁₅NS = 373.47) |
| Sub 3-117 | m/z = 323.08(C₂₂H₁₃NS = 323.41) |
| Sub 3-118 | m/z = 373.09(C₂₆H₁₅NS = 373.47) |
| Sub 3-119 | m/z = 323.08(C₂₂H₁₃NS = 323.41) |
| Sub 3-120 | m/z = 323.08(C₂₂H₁₃NS = 323.41) |
| Sub 3-121 | m/z = 373.09(C₂₆H₁₅NS = 373.47) |
| Sub 3-122 | m/z = 373.09(C₂₆H₁₅NS = 373.47) |
| Sub 3-123 | m/z = 373.09(C₂₆H₁₅NS = 373.47) |
| Sub 3-124 | m/z = 373.09(C₂₆H₁₅NS = 373.47) |
| Sub 3-125 | m/z = 373.09(C₂₆H₁₅NS = 373.47) |
| Sub 3-126 | m/z = 373.09(C₂₆H₁₅NS = 373.47) |
| Sub 3-127 | m/z = 307.1(C₂₂H₁₃NO = 307.35) |
| Sub 3-128 | m/z = 307.1(C₂₂H₁₃NO = 307.35) |
| Sub 3-129 | m/z = 307.1(C₂₂H₁₃NO = 307.35) |
| Sub 3-130 | m/z = 307.1(C₂₂H₁₃NO = 307.35) |
| Sub 3-131 | m/z = 307.1(C₂₂H₁₃NO = 307.35) |
| Sub 3-132 | m/z = 357.12(C₂₆H₁₅NO = 357.41) |
| Sub 3-133 | m/z = 357.12(C₂₆H₁₅NO = 357.41) |
| Sub 3-134 | m/z = 357.12(C₂₆H₁₅NO = 357.41) |
| Sub 3-135 | m/z = 307.1(C₂₂H₁₃NO = 307.35) |
| Sub 3-136 | m/z = 357.12(C₂₆H₁₅NO = 357.41) |
| Sub 3-137 | m/z = 307.1(C₂₂H₁₃NO = 307.35) |
| Sub 3-138 | m/z = 357.12(C₂₆H₁₅NO = 357.41) |
| Sub 3-139 | m/z = 357.12(C₂₆H₁₅NO = 357.41) |
| Sub 3-140 | m/z = 407.1 3(C₃₀H₁₇NO = 407.47) |
| Sub 3-141 | m/z = 357.12(C₂₆H₁₅NO = 357.41) |
| Sub 3-142 | m/z = 357.12(C₂₆H₁₅NO = 357.41) |
| Sub 3-143 | m/z = 357.12(C₂₆H₁₅NO = 357.41) |
| Sub 3-144 | m/z = 399.11(C₂₈H₁₇NS = 399.51) |
| Sub 3-145 | m/z = 490.15(C₃₄H₂₂N₂S = 490.62) |
| Sub 3-146 | m/z = 450.12(C₃₁ H₁₈N₂S = 450.56) |
| Sub 3-147 | m/z = 564.17(C₄₀H₂₄N₂S = 564.71) |
| Sub 3-148 | m/z = 555.11(C₃₈H₂₁NS₂ = 555.71) |
| Sub 3-149 | m/z = 692.2(C₄₈H₂₈N₄S = 692.84) |
| Sub 3-150 | m/z = 363.11(C₂₅H₁₇NS = 363.48) |
| Sub 3-151 | m/z = 397.15(C₂₉H₁₉NO = 397.48) |
| Sub 3-152 | m/z = 474.17(C₃₄H₂₂N₂O = 474.56) |
| Sub 3-153 | m/z = 337.11(C₂₃H₁₅NO₂ = 337.38) |
| Sub 3-154 | m/z = 598.2(C₄₄H₂₆N₂O = 598.71) |
| Sub 3-155 | m/z = 538.18(C₃₇H₂₂N₄O = 538.61) |
| Sub 3-156 | m/z = 438.21(C₃₂H₂₆N₂ = 438.57) |
| Sub 3-157 | m/z = 625.25(C₄₆H₃₁N₃ = 625.78) |
| Sub 3-158 | m/z = 623.24(C₄₆H₂₉N₃ = 623.76) |
| Sub 3-159 | m/z = 612.23(C₄₄H₂N₄ = 612.74) |
| Sub 3-160 | m/z = 323.08(C₂₂H₁₃NS = 323.41) |
| Sub 3-161 | m/z = 323.08(C₂₂H₁₃NS = 323.41) |
| Sub 3-162 | m/z = 307.1(C₂₂H₁₃NO = 307.35) |
| Sub 3-163 | m/z = 307.1(C₂₂H₁₃NO = 307.35) |
| Sub 3-164 | m/z = 382.15(C₂₈H₁₈N₂ = 382.47) |
| Sub 3-165 | m/z = 323.08(C₂₂H₁₃NS = 323.41) |
| Sub 3-166 | m/z = 307.1(C₂₂H₁₃NO = 307.35) |
| Sub 3-167 | m/z = 373.09(C₂₆H₁₅NS = 373.47) |
| Sub 3-168 | m/z = 323.08(C₂₂H₁₃NS = 323.41) |
| Sub 3-169 | m/z = 357.12(C₂₆H₁₅NO = 357.41) |
| Sub 3-170 | m/z = 373.09(C₂₆H₁₅NS = 373.47) |
| Sub 3-171 | m/z = 357.12(C₂₆H₁₅NO = 357.41) |
| Sub 3-172 | m/z = 373.09(C₂₆H₁₅NS = 373.47) |
| Sub 3-173 | m/z = 407.13(C₃₀H₁₇NO = 407.47) |
| Sub 3-174 | m/z = 373.09(C₂₆H₁₅NS = 373.47) |
| Sub 3-175 | m/z = 357.12(C₂₆H₁₅NO = 357.41) |
| Sub 3-176 | m/z = 662.25(C₄₈H₃₀N₄ = 662.8) |
| Sub 3-177 | m/z = 373.09(C₂₆H₁₅NS = 373.47) |
| Sub 3-178 | m/z = 357.12(C₂₆H₁₅NO = 357.41) |
| Sub 3-179 | m/z = 432.16(C₃₂H₂₀N₂ = 432.53) |
| Sub 3-180 | m/z = 323.08(C₂₂H₁₃NS = 323.41) |
| Sub 3-181 | m/z = 307.1(C₂₂H₁₃NO = 307.35) |
| Sub 3-182 | m/z = 432.16(C₃₂H₂₀N₂ = 432.53) |
| Sub 3-183 | m/z = 373.09(C₂₆H₁₅NS = 373.47) |
| Sub 3-184 | m/z = 357.12(C₂₆H₁₅NO = 357.41) |
| Sub 3-185 | m/z = 432.16(C₃₂H₂₀N₂ = 432.53) |
| Sub 3-186 | m/z = 373.09(C₂₆H₁₅NS = 373.47) |
| Sub 3-187 | m/z = 357.12(C₂₆H₁₅NO = 357.41) |
| Sub 3-188 | m/z = 432.16(C₃₂H₂₀N₂ = 432.53) |
| Sub 3-189 | m/z = 373.09(C₂₆H₁₅NS = 373.47) |
| Sub 3-190 | m/z = 357.12(C₂₆H₁₅NO = 357.41) |
| Sub 3-191 | m/z = 382.15(C₂₈H₁₈N₂ = 382.47) |
| Sub 3-192 | m/z = 323.08(C₂₂H₁₃NS = 323.41) |
| Sub 3-193 | m/z = 307.1(C₂₂H₁₃NO = 307.35) |
| Sub 3-194 | m/z = 432.16(C₃₂H₂₀N₂ = 432.53) |
| Sub 3-195 | m/z = 323.08(C₂₂H₁₃NS = 323.41) |
| Sub 3-196 | m/z = 307.1(C₂₂H₁₃NO = 307.35) |
| Sub 3-197 | m/z = 432.16(C₃₂H₂₀N₂ = 432.53) |
| Sub 3-198 | m/z = 373.09(C₂₆H₁₅NS = 373.47) |
| Sub 3-199 | m/z = 357.12(C₂₆H₁₅NO = 357.41) |
| Sub 3-200 | m/z = 616.17(C₄₂H₂₄N₄S = 616.74) |
| Sub 3-201 | m/z = 323.08(C₂₂H₁₃NS = 323.41) |
| Sub 3-202 | m/z = 307.1(C₂₂H₁₃NO = 307.35) |
| Sub 3-203 | m/z = 373.09(C₂₆H₁₅NS = 373.47) |
| Sub 3-204 | m/z = 432.16(C₃₂H₂₀N₂ = 432.53) |
| Sub 3-205 | m/z = 373.09(C₂₆H₁₅NS = 373.47) |
| Sub 3-206 | m/z = 357.12(C₂₆H₁₅NO = 357.41) |
| Sub 3-207 | m/z = 432.16(C₃₂H₂₀N₂ = 432.53) |
| Sub 3-208 | m/z = 432.16(C₃₂H₂₀N₂ = 432.53) |
| Sub 3-209 | m/z = 548.23(C₄₁H₂₈N₂ = 548.69) |
| Sub 3-210 | m/z = 382.15(C₂₈H₁₈N₂ = 382.47) |
| Sub 3-211 | m/z = 307.1(C₂₂H₁₃NO = 307.35) |
| Sub 3-212 | m/z = 373.09(C₂₆H₁₅NS = 373.47) |
| Sub 3-213 | m/z = 307.1(C₂₂H₁₃NO = 307.35) |
| Sub 3-214 | m/z = 382.15(C₂₈H₁₈N₂ = 382.47) |
| Sub 3-215 | m/z = 323.08(C₂₂H₁₃NS = 323.41) |
| Sub 3-216 | m/z = 399.11(C₂₈H₁₇NS = 399.51) |
| Sub 3-217 | m/z = 490.15(C₃₄H₂₂N₂S = 490.62) |
| Sub 3-218 | m/z = 468.11(C₃₁ H₁₇FN₂S = 468.55) |
| Sub 3-219 | m/z = 564.17(C₄₀H₂₄N₂S = 564.71) |
| Sub 3-220 | m/z = 555.11(C₃₈H₂₁NS₂ = 555.71) |
| Sub 3-221 | m/z = 363.11(C₂₅H₁₇NS = 363.48) |
| Sub 3-222 | m/z = 692.2(C₄₈H₂₈N₄S = 692.84) |
| Sub 3-223 | m/z = 337.11(C₂₃H₁₅NO₂ = 337.38) |
| Sub 3-224 | m/z = 524.19(C₃₈H₂₄N₂O = 524.62) |
| Sub 3-225 | m/z = 598.2(C₄₄H₂N₂O = 598.71) |
| Sub 3-226 | m/z = 538.18(C₃₇H₂₂N₄O = 538.61) |
| Sub 3-227 | m/z = 438.21(C₃₂H₂₆N₂ = 438.57) |
| Sub 3-228 | m/z = 598.2(C₄₄H₂₆N₂O = 598.71) |
| Sub 3-229 | m/z = 307.1(C₂₂H₁₃NO = 307.35) |
| Sub 3-230 | m/z = 357.12(C₂₆H₁₅NO = 357.41) |
| Sub 3-231 | m/z = 332.13(C₂₄H₁₆N₂ = 332.41) |
| Sub 3-232 | m/z = 273.06(C₁₈H₁₁NS = 273.35) |
| Sub 3-233 | m/z = 257.08(C₁₈H₁₁NO = 257.29) |
| Sub 3-234 | m/z = 488.16(C₃₃H₂₀N₄O = 488.55) |
| Sub 3-235 | m/z = 573.22(C₄₂H₂₇N₃ = 573.7) |
| Sub 3-236 | m/z = 498.17(C₃₆H₂₂N₂O = 498.59) |
| Sub 3-237 | m/z = 409.15(C₃₀H₁₉NO = 409.49) |
| Sub 3-238 | m/z = 511.17(C₃₆H₂₁N₃O = 511.58) |
| Sub 3-239 | m/z = 332.13(C₂₄H₁₆N₂ = 332.41) |
| Sub 3-240 | m/z = 349.09(C₂₄H₁₅NS = 349.45) |
| Sub 3-241 | m/z = 332.13(C₂₄H₁₆N₂ = 332.41) |
| Sub 3-242 | m/z = 333.12(C₂₄H₁₅NO = 333.39) |

### II. Synthesis Example of Sub 4

Sub 4 of Reaction Scheme 4 may be synthesized by the reaction route of Reaction Scheme 6, but is not limited thereto. In this case, Hal¹ is I, Br or Cl, and Hal² is Br or Cl.

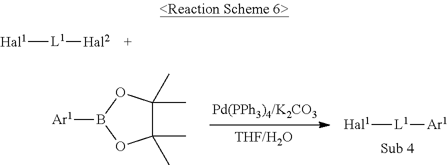

### 1. Synthesis Example of Sub 4-35

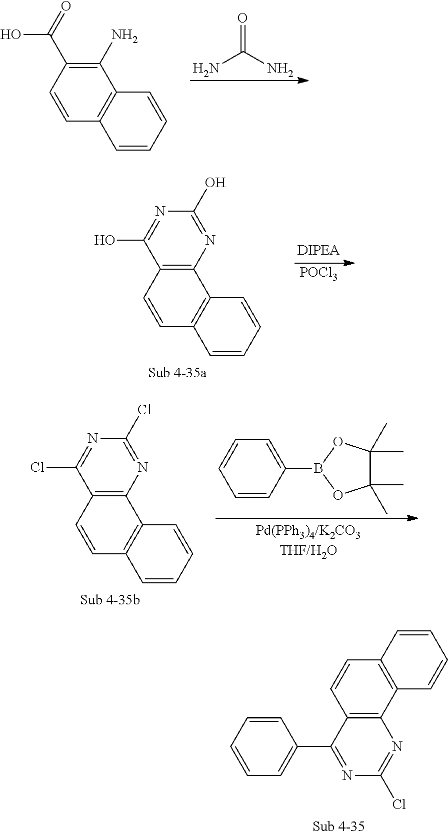

### (1) Synthesis of Sub 4-35a

The starting material, 1-amino-2-naphthoic acid (CAS Registry Number: 4919-43-1) (75.11 g, 401.25 mmol) was placed in a round-bottom flask with urea (CAS Registry Number: 57-13-6) (168.69 g, 2808.75 mmol) and stirred at 160° C.

After confirming the reaction by TLC, cooled to 100° C., water (200 ml) was added, and the mixture was stirred for 1 hour. When the reaction was completed, the resulting solid was filtered under reduced pressure, washed with water and dried to obtain 63.86 g (yield: 75%) of the product.

### (2) Synthesis of Sub 4-35b

The obtained Sub 4-35a (63.86 g, 300.94 mmol) was dissolved in POCl₃ (200 ml) in a round-bottom flask at room temperature, and then N,N-Diisopropylethylamine (97.23 g, 752.36 mmol) was slowly added dropwise, followed by stirring at 90° C. After the reaction was completed, ice water (500 ml) was added after concentration, and the mixture was stirred at room temperature for 1 hour. The resulting solid was filtered under reduced pressure and dried to obtain 67.47 g of a product (yield: 90%).

### (3) Synthesis of Sub 4-35

The obtained Sub 4-35b (67.47 g, 270.86 mmol) was dissolved in THE (950 ml) in a round-bottom flask, 4,4,5,5-tetramethyl-2-phenyl-1,3,2-dioxaborolane (CAS Registry Number: 24388-23-6) (60.80 g, 297.94 mmol), Pd(PPh₃)₄ (12.52 g, 10.83 mmol), K₂CO₃ (112.30 g, 812.57 mmol), Water (475 ml) were added and stirred at 90° C. After the reaction was completed, the mixture was extracted with CH₂Cl₂ and water, the organic layer was dried over MgSO₄, concentrated, and the resulting compound was recrystallized by silicagel column to obtain 44.89 g (yield: 57%) of the product.

### 2. Synthesis Example of Sub 4-40

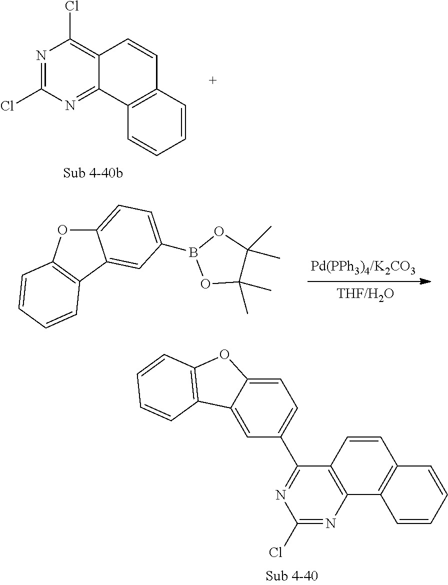

To the starting material, Sub 4-40b (19 g, 76.28 mmol), 2-(dibenzo[b,d]furan-2-yl)-4,4,5,5-tetramethyl-1,3,2-dioxaborolane (CAS Registry Number: 947770-80-1) (22.44 g, 76.28 mmol), Pd(PPh₃)₄ (1.32 g, 1.14 mmol), K₂CO₃ (15.81 g, 114.42 mmol), THE (336 ml), Water (168 ml) were added and 15.69 g (yield: 54%) of the product was obtained using the synthesis method of Sub 4-35.

### 3. Synthesis Example of Sub 4-43

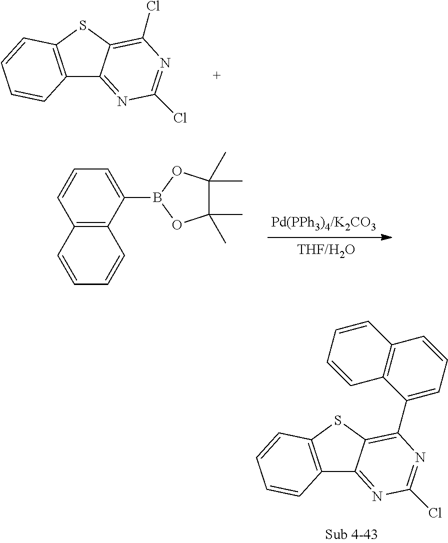 [SMILES: Clc1nc(Cl)c2sc3ccccc3c2n1 ; CC1(C)OB(c2cccc3ccccc23)OC1(C)C ; Clc1nc(-c2cccc3ccccc23)c2sc3ccccc3c2n1]

To the starting material, 2,4-dichlorobenzo[4,5]thieno[3,2-d]pyrimidine (CAS Registry Number: 160199-05-3) (32.01 g, 125.47 mmol), 4,4,5,5-tetramethyl-2-(naphthalen-1-yl)-1,3,2-dioxaborolane (CAS Registry Number: 68716-52-9) (35.07 g, 138.02 mmol), Pd(PPh₃)₄ (5.80 g, 5.02 mmol), K₂CO₃ (52.02 g, 376.41 mmol), THE (336 ml), Water (168 ml) were added and 19.58 g (yield: 45%) of the product was obtained using the synthesis method of Sub 4-35.

The compound belonging to Sub 4 may be the following compounds, but is not limited thereto, and Table 5 shows FD-MS (Field Desorption-Mass Spectrometry) values of the compounds belonging to Sub 4.

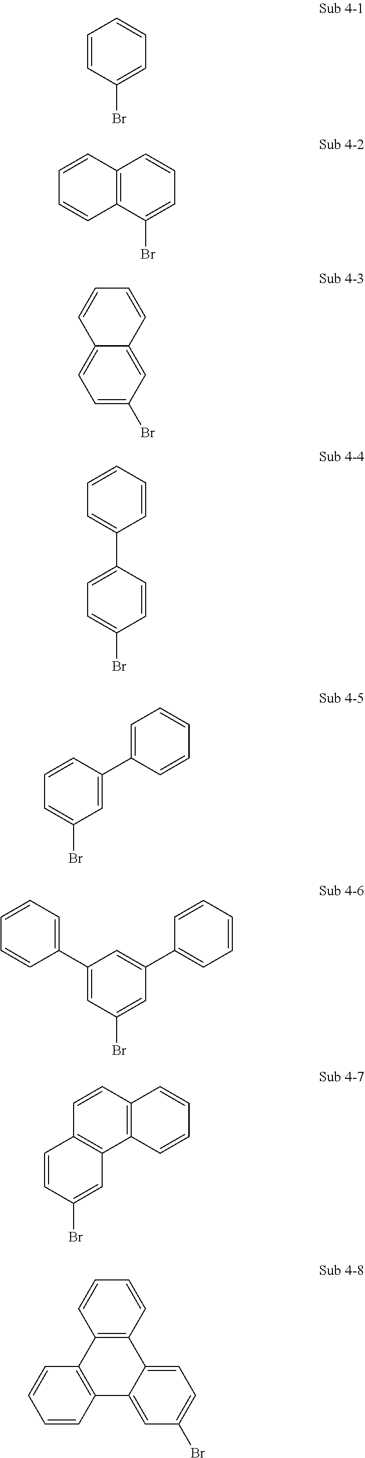

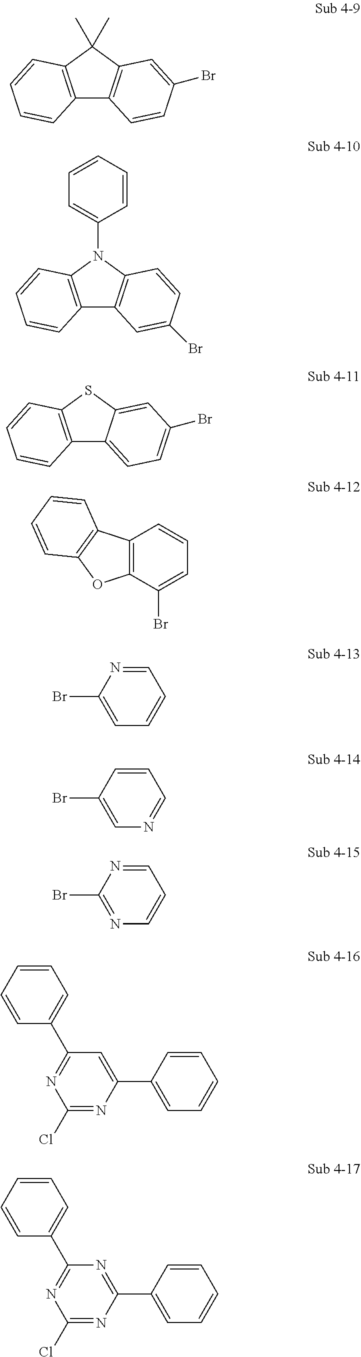

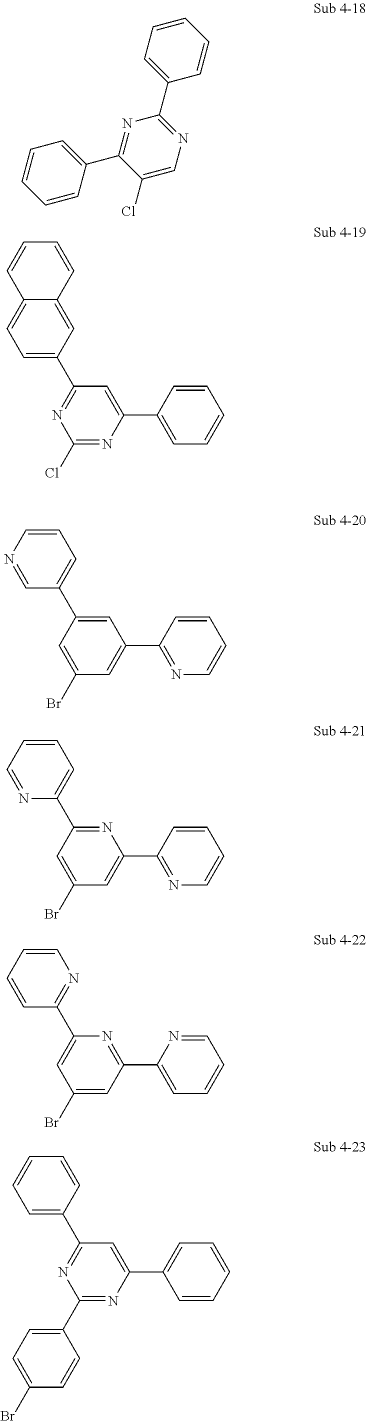

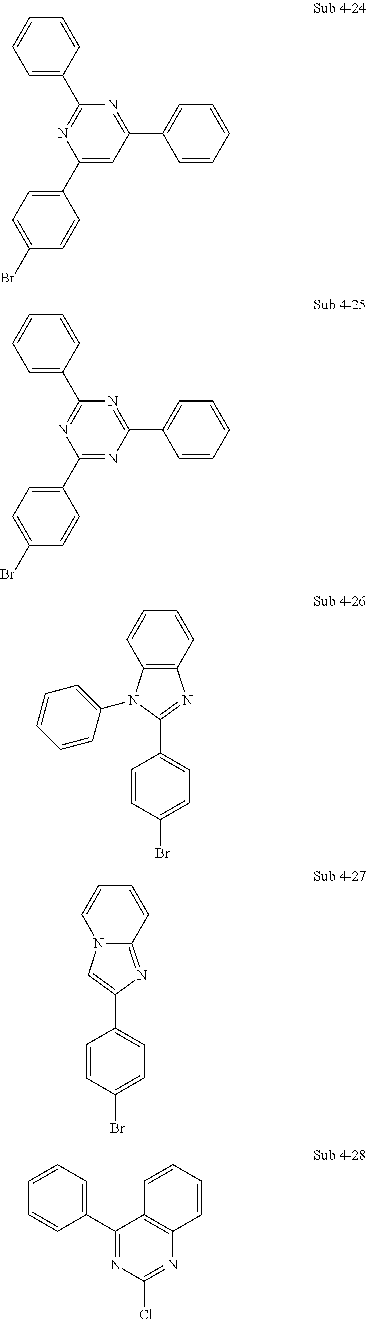

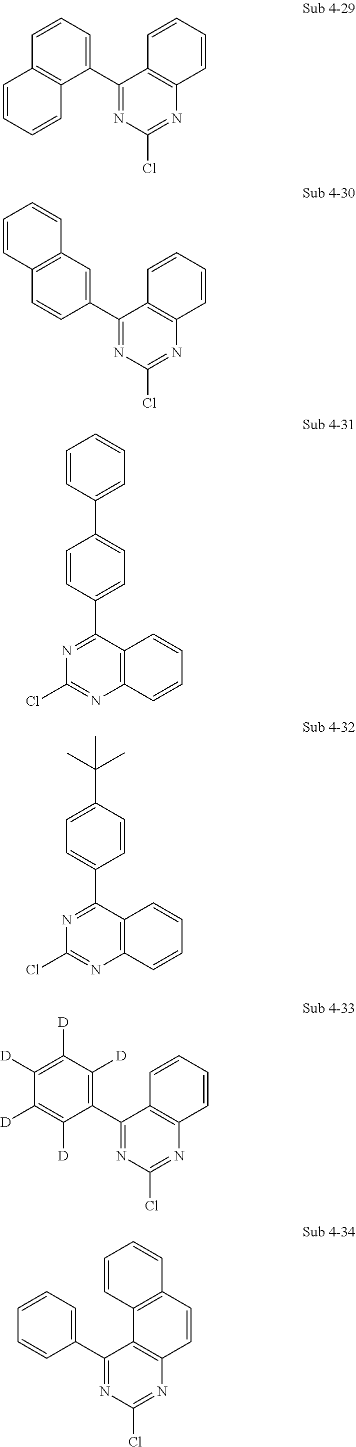

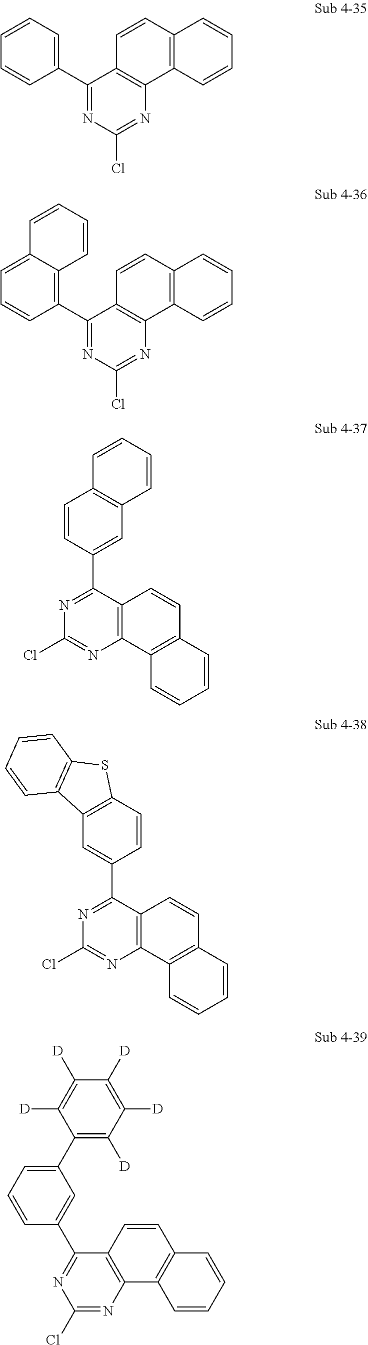

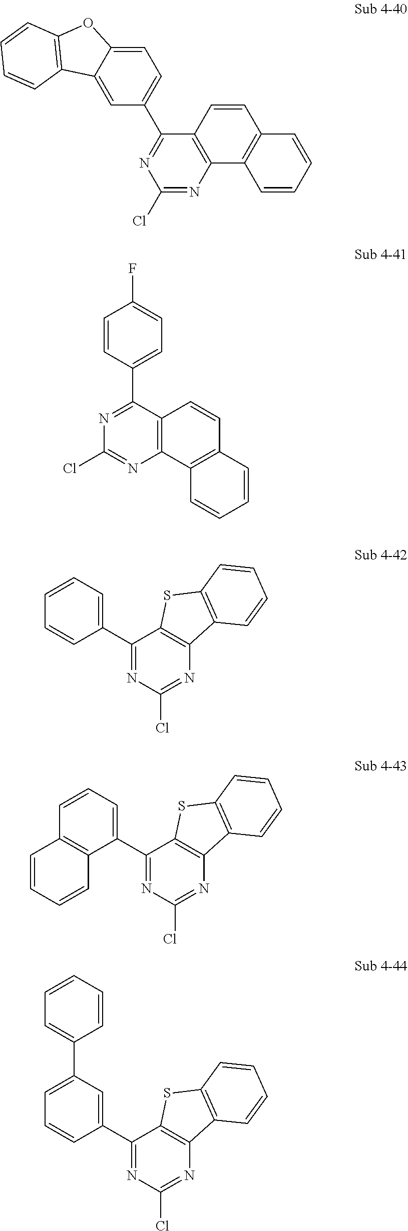

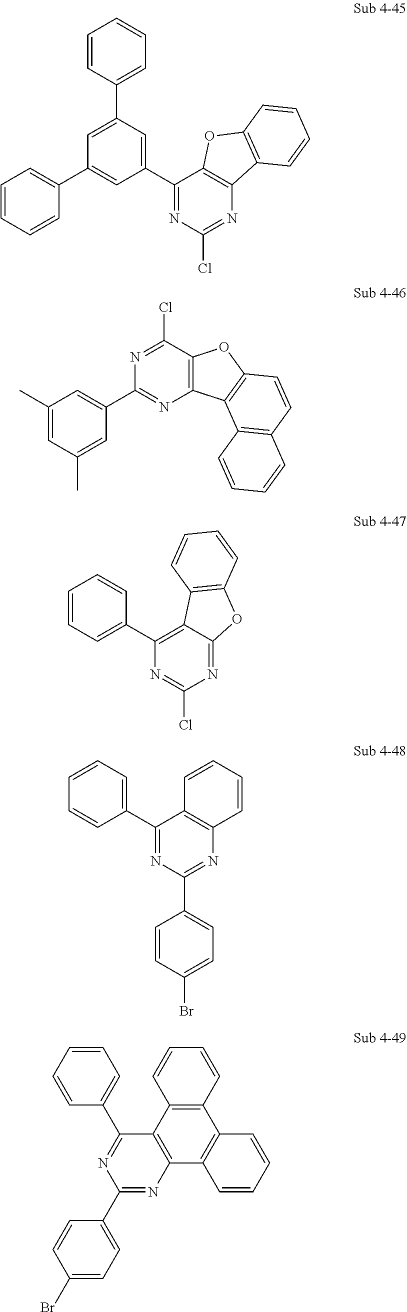

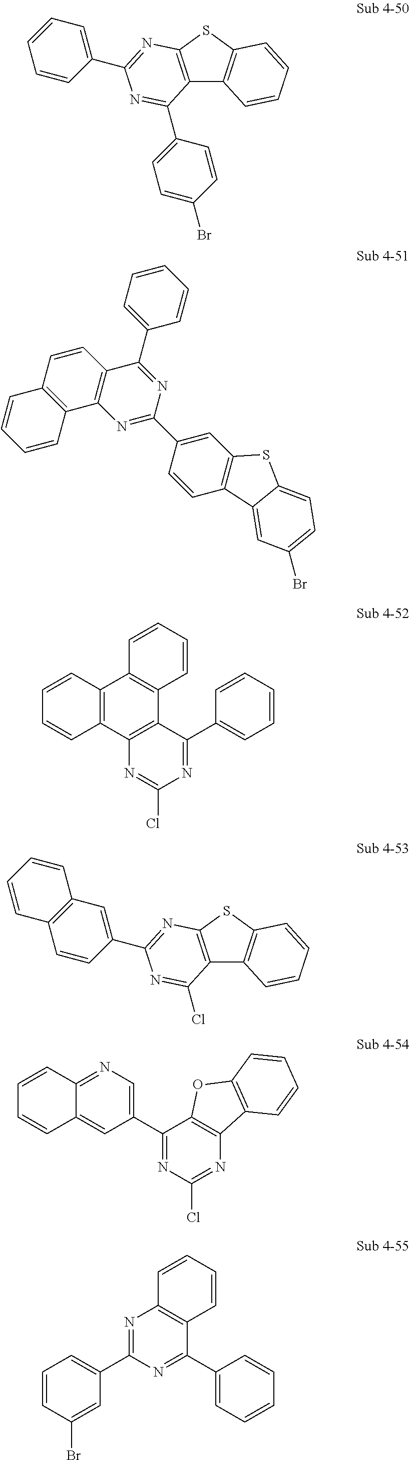

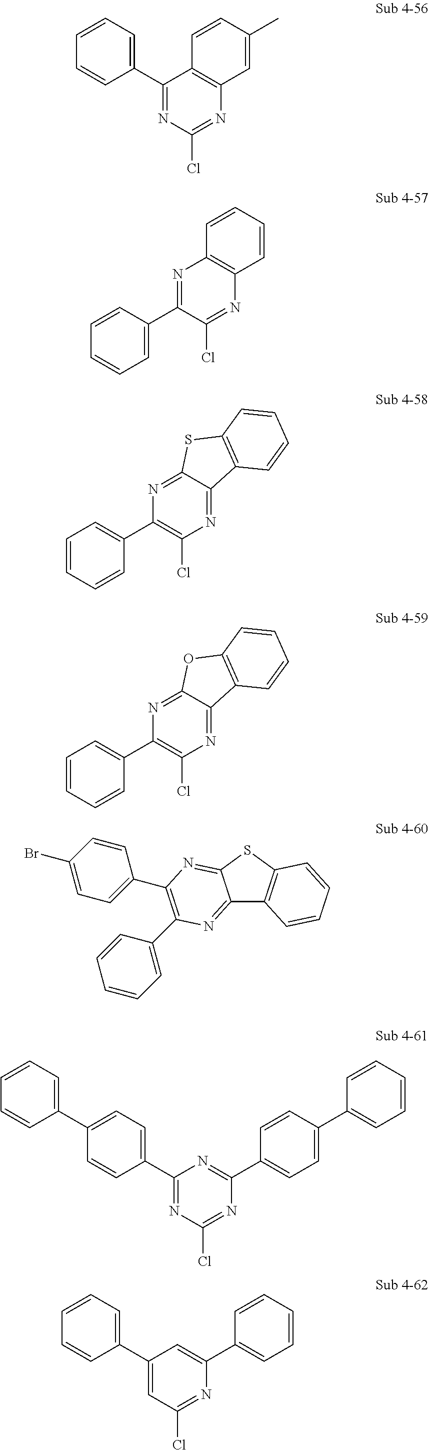

TABLE 5

| compound | FD-MS |
|---|---|
| Sub 4-1 | m/z = 155.96(C₆H₅Br = 157.01) |
| Sub 4-2 | m/z = 205.97(C₁₀H₇Br = 207.07) |
| Sub 4-3 | m/z = 205.97(C₁₀H₇Br = 207.07) |
| Sub 4-4 | m/z = 231.99(C₁₂H₉Br = 233.11) |
| Sub 4-5 | m/z = 231.99(C₁₂H₉Br = 233.11) |
| Sub 4-6 | m/z = 308.02(C₁₈H₁₃Br = 309.21) |
| Sub 4-7 | m/z = 255.99(C₁₄H₉Br = 257.13) |
| Sub 4-8 | m/z = 306.00(C₁₈H₁₁Br = 307.19) |
| Sub 4-9 | m/z = 272.02(C₁₅H₁₃Br = 273.17) |
| Sub 4-10 | m/z = 321.02(C₁₈H₁₂BrN = 322.21) |
| Sub 4-11 | m/z = 261.95(C₁₂H₇BrS = 263.15) |
| Sub 4-12 | m/z = 245.97(C₁₂H₇BrO = 247.09) |
| Sub 4-13 | m/z = 156.95(C₅H₄BrN = 158.00) |
| Sub 4-14 | m/z = 156.95(C₅H₄BrN = 158.00) |
| Sub 4-15 | m/z = 157.95(C₄H₃BrN₂ = 158.99) |
| Sub 4-16 | m/z = 266.06(C₁₆H₁₁ClN₂ = 266.72) |
| Sub 4-17 | m/z = 267.06(C₁₅H₁₀ClN₃ = 267.72) |
| Sub 4-18 | m/z = 266.06(C₁₆H₁₁ClN₂ = 266.72) |
| Sub 4-19 | m/z = 316.08(C₂₀H₁₃ClN₂ = 316.79) |
| Sub 4-20 | m/z = 310.01(C₁₆H₁₁BrN₂ = 311.18) |
| Sub 4-21 | m/z = 311.01(C₁₅H₁₀BrN₃ = 312.17) |
| Sub 4-22 | m/z = 311.01(C₁₅H₁₀BrN₃ = 312.17) |
| Sub 4-23 | m/z = 386.04(C₂₂H₁₅BrN₂ = 387.28) |
| Sub 4-24 | m/z = 386.04(C₂₂H₁₅BrN₂ = 387.28) |
| Sub 4-25 | m/z = 387.04(C₂₁H₁₄BrN₃ = 388.27) |
| Sub 4-26 | m/z = 348.03(C₁₉H₁₃BrN₂ = 349.23) |
| Sub 4-27 | m/z = 273.13(C₁₃H₉BrN₂ = 273.13) |
| Sub 4-28 | m/z = 240.05(C₁₄H₉ClN₂ = 240.69 |
| Sub 4-29 | m/z = 290.06(C₁₈H₁₁ClN₂ = 290.75) |
| Sub 4-30 | m/z = 290.06(C₁₈H₁₁ClN₂ = 290.75) |
| Sub 4-31 | m/z = 316.08(C₂₀H₁₃ClN₂ = 316.79) |
| Sub 4-32 | m/z = 296.11(C₁₈H₁₇ClN₂ = 296.80) |
| Sub 4-33 | m/z = 245.08(C₁₄H₄D₅ClN₂ = 245.72) |
| Sub 4-34 | m/z = 290.06(C₁₈H₁₁ClN₂ = 290.75) |
| Sub 4-35 | m/z = 290.06(C₁₈H₁₁ClN₂ = 290.75) |
| Sub 4-36 | m/z = 340.08(C₂₂H₁₃ClN₂ = 340.81) |
| Sub 4-37 | m/z = 340.08(C₂₂H₁₃ClN₂ = 340.81) |
| Sub 4-38 | m/z = 396.05(C₂₄H₁₃ClN₂S = 396.89) |
| Sub 4-39 | m/z = 371.12(C₂₄H₁₀D₅ClN₂ = 371.88) |
| Sub 4-40 | m/z = 380.07(C₂₄H₁₃ClN₂0 = 380.83) |
| Sub 4-41 | m/z = 308.05(C₁₈H₁₀ClFN₂ = 308.74) |
| Sub 4-42 | m/z = 296.02(C₁₆H₉ClN₂S = 296.77) |
| Sub 4-43 | m/z = 346.03(C₂₀H₁₁ClN₂S = 346.83) |
| Sub 4-44 | m/z = 372.05(C₂₂H₁₃ClN₂S = 372.87) |
| Sub 4-45 | m/z = 432.10(C₂₈H₁₇ClN₂O = 432.91) |
| Sub 4-46 | m/z = 358.09(C₂₂H₁₅ClN₂O = 358.83) |
| Sub 4-47 | m/z = 280.04(C₁₆H₉ClN₂0 = 280.71) |
| Sub 4-48 | m/z = 360.03(C₂₀H₁₃BrN₂ = 361.24) |
| Sub 4-49 | m/z = 460.06(C₂₈H₁₇BrN₂ = 461.36) |
| Sub 4-50 | m/z = 416.00(C₂₂H₁₃BrN₂S = 417.32) |
| Sub 4-51 | m/z = 516.03(C₃₀H₁₇BrN₂S = 517.44) |
| Sub 4-52 | m/z = 340.08(C₂₂H₁₃ClN₂ = 340.81) |
| Sub 4-53 | m/z = 346.03(C₂₀H₁₁ClN₂S = 346.83) |
| Sub 4-54 | m/z = 331.05(C₁₉H₁₀ClN₃O = 331.76) |
| Sub 4-55 | m/z = 360.03(C₂₀H₁₃BrN₂ = 361.24) |
| Sub 4-56 | m/z = 254.06(C₁₅H₁₁ClN₂ = 254.72) |
| Sub 4-57 | m/z = 240.05(C₁₄H₉ClN₂ = 240.69) |
| Sub 4-58 | m/z = 296.02(C₁₆H₉ClN₂S = 296.77) |
| Sub 4-59 | m/z = 280.04(C₁₆H₉ClN₂0 = 280.71) |
| Sub 4-60 | m/z = 416.00(C₂₂H₁₃BrN₂S = 417.32) |
| Sub 4-61 | m/z = 419.12(C₂₇H₁₈ClN₃ = 419.91) |
| Sub 4-62 | m/z = 265.07(C₁₇H₁₂ClN = 265.74) |

### III. Synthesis Example of Final Product 2

After dissolving Sub 3 (1 equiv.) with Toluene in a round bottom flask, Sub 4 (1.1 equiv), Pd₂(dba)₃ (0.03 equiv), P(t-Bu)₃ (0.1 equiv), NaOt-Bu (3 equiv) were added and stirred at 100° C. After the reaction was completed, extracted with CH₂Cl₂ and water, the organic layer was dried over MgSO₄, concentrated, and the resulting compound was recrystallized using silicagel column to obtain Final product 1.

### 1. Synthesis Example 2-1

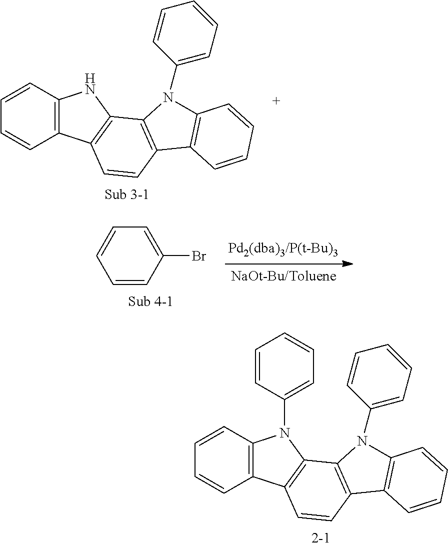 [SMILES: c1ccc(-n2c3ccccc3c3ccc4c5ccccc5[nH]c4c32)cc1 ; Brc1ccccc1 ; c1ccc(-n2c3ccccc3c3ccc4c5ccccc5n(-c5ccccc5)c4c32)cc1]

After Sub 3-1 (10 g, 30.08 mmol) was placed in a round-bottom flask and dissolved with Toluene (302 mL), Sub 4-1 (5.19 g, 33.09 mmol), Pd₂(dba)₃ (0.83 g, 0.90 mmol), P(t-Bu)₃ (0.61 g, 3.01 mmol), NaOt-Bu (8.67 g, 90.24 mmol) were added and stirred at 100° C. After the reaction was completed, the organic layer was extracted with CH₂Cl₂ and water, dried over MgSO₄, concentrated, and the resulting compound was recrystallized using silicagel column to obtain 8.14 g (yield: 66%) of the product.

### 2. Synthesis Example 2-23

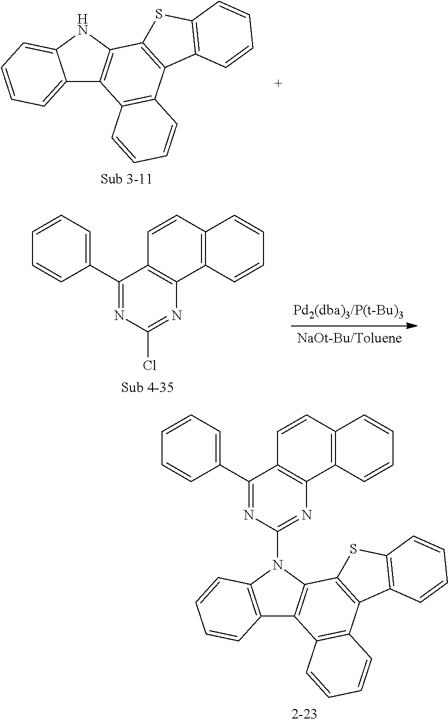 [SMILES: c1ccc2c(c1)[nH]c1c3sc4ccccc4c3c3ccccc3c21 ; Clc1nc(-c2ccccc2)c2ccc3ccccc3c2n1 ; c1ccc(-c2nc(-n3c4ccccc4c4c5ccccc5c5c6ccccc6sc5c43)nc3c2ccc2ccccc23)cc1]

Sub 3-11 (10 g, 30.92 mmol), Toluene (325 mL), Sub 4-35 (9.89 g, 34.01 mmol), Pd₂(dba)₃ (0.85 g, 0.93 mmol), P(t-Bu)₃ (0.38 g, 1.86 mmol), NaOt-Bu (8.91 g, 92.76 mmol) were carried out in Synthesis method of Sub 2-1 to obtain a final product (13.04 g, 73%).

### 3. Synthesis Example 2-53

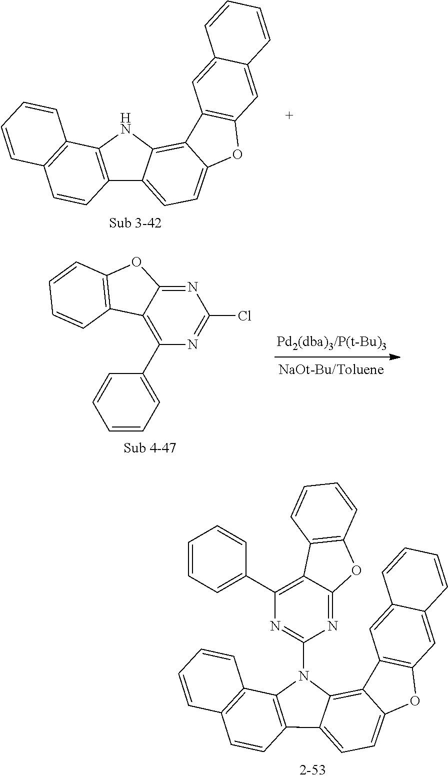 [SMILES: c1ccc2cc3c(cc2c1)oc1ccc2c4ccc5ccccc5c4[nH]c2c13 ; Clc1nc(-c2ccccc2)c2c(n1)oc1ccccc12 ; c1ccc(-c2nc(-n3c4c5ccccc5ccc4c4ccc5oc6cc7ccccc7cc6c5c43)nc3oc4ccccc4c23)cc1]

Sub 3-36 (10 g, 27.98 mmol), Toluene (294 mL), Sub 4-47 (8.64 g, 30.78 mmol), Pd₂(dba)₃ (0.77 g, 0.84 mmol), P(t-Bu)₃ (0.34 g, 1.68 mmol), NaOt-Bu (8.07 g, 83.94 mmol) were carried out in Synthesis method 2-1 to obtain a final product (11.45 g, 68%).

### 4. Synthesis Example 2-92

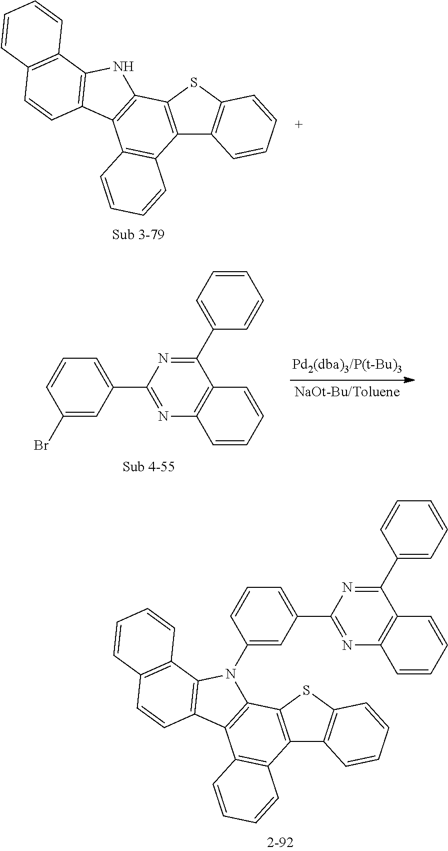 [SMILES: c1ccc2c(c1)ccc1c2[nH]c2c3sc4ccccc4c3c3ccccc3c12 ; Brc1cccc(-c2nc(-c3ccccc3)c3ccccc3n2)c1 ; c1ccc(-c2nc(-c3cccc(-n4c5c6ccccc6ccc5c5c6ccccc6c6c7ccccc7sc6c54)c3)nc3ccccc23)cc1]

Sub 3-79 (10 g, 26.78 mmol), Toluene (281 mL), Sub 4-55 (9.67 g, 26.78 mmol), Pd₂(dba)₃ (0.37 g, 0.40 mmol), P(t-Bu)₃ (0.16 g, 0.80 mmol), NaOt-Bu (3.86 g, 40.16 mmol) were carried out in Synthesis method 2-1 to obtain a final product (12.25 g, 70%).

### 5. Synthesis Example 3-21

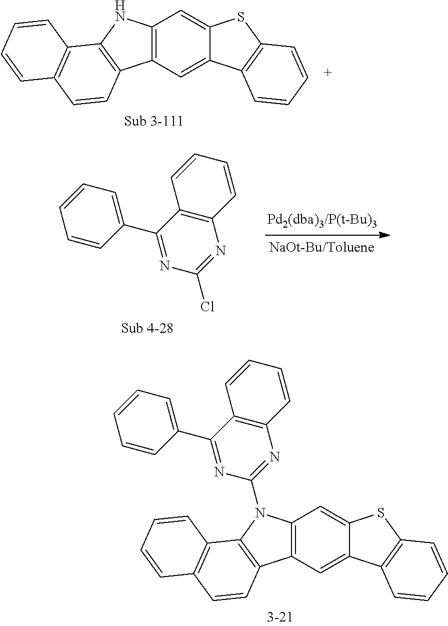 [SMILES: c1ccc2c(c1)ccc1c3cc4c(cc3[nH]c21)sc1ccccc14 ; Clc1nc(-c2ccccc2)c2ccccc2n1 ; c1ccc(-c2nc(-n3c4cc5sc6ccccc6c5cc4c4ccc5ccccc5c43)nc3ccccc23)cc1]

After dissolving Sub 3-111 (3.90 g, 12.06 mmol) in toluene (127 ml), Sub 4-28 (2.90 g, 12.06 mmol), Pd₂(dba)₃ (0.33 g, 0.36 mmol), P(t-Bu)₃ (0.24 g, 1.21 mmol), NaOt-Bu (3.48 g, 36.18 mmol) were carried out in Synthesis method 2-1 to obtain a final product (6.61 g, 76%).

### 6. Synthesis Example 4-1

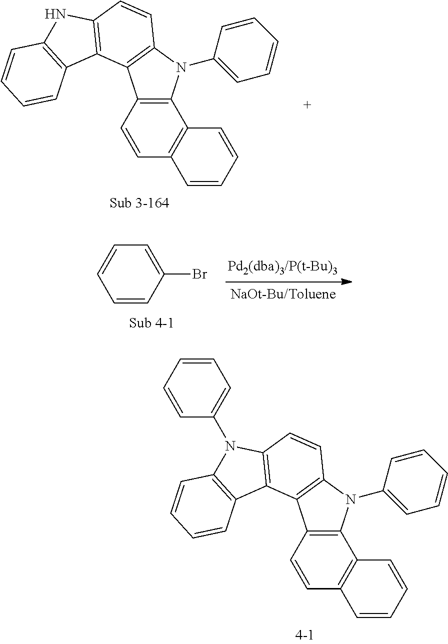 [SMILES: c1ccc(-n2c3ccc4[nH]c5ccccc5c4c3c3ccc4ccccc4c32)cc1 ; Brc1ccccc1 ; c1ccc(-n2c3ccccc3c3c4c5ccc6ccccc6c5n(-c5ccccc5)c4ccc32)cc1]

After dissolving Sub 3-164 (5.3 g, 16.39 mmol) in toluene (172 ml), Sub 4-1 (4.86 g, 16.39 mmol), Pd₂(dba)₃ (0.45 g, 0.49 mmol), P(t-Bu)₃ (0.20 g, 0.98 mmol), NaOt-Bu (4.72 g, 49.16 mmol) were carried out in Synthesis method 2-1 to obtain a product (7.56 g, 79%).

### 7. Synthesis Example 4-37

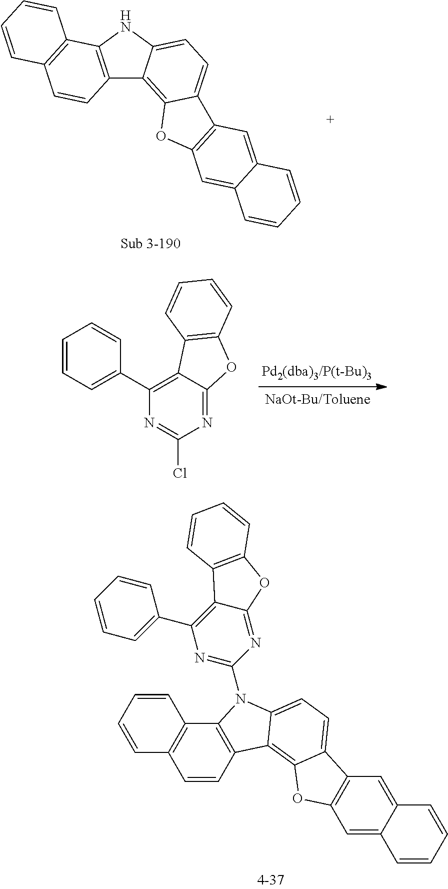

Sub 3-190 (7 g, 19.59 mmol), toluene (206 ml), Sub 4-47 (5.50 g, 19.59 mmol), Pd₂(dba)₃ (0.54 g, 0.59 mmol), P(t-Bu)₃ (0.24 g, 1.18 mmol), NaOt-Bu (5.65 g, 58.76 mmol) were carried out in Synthesis method 2-1 to obtain a product (7.54 g, 64%).

Table 6 shows FD-MS (Field Desorption-Mass Spectrometry) values of compounds belonging to Final product 2.

TABLE 6

| compound | FD-MS |
|---|---|
| 2-1 | m/z = 408.16(C₃₀H₂₀N₂ = 408.5) |
| 2-2 | m/z = 536.2(C₃₈H₂₄N₄ = 536.64) |
| 2-3 | m/z = 449.12(C₃₂H₁₉NS = 449.57) |
| 2-4 | m/z = 461.15(C₃₂H₁₉N₃O = 461.52) |
| 2-5 | m/z = 483.16(C₃₆H₂₁NO = 483.57) |
| 2-6 | m/z = 457.07(C₂₈H₁₅N₃S₂ = 457.57) |
| 2-7 | m/z = 593.16(C₄₀H₂₃N₃OS = 593.7) |
| 2-8 | m/z = 524.23(C₃₉H₂₈N₂ = 524.67) |
| 2-9 | m/z = 554.16(C₃₇H₂₂N₄S = 554.67) |
| 2-10 | m/z = 693.19(C₄₈H₂₇N₃OS = 693.82) |
| 2-11 | m/z = 586.22(C₄₂H₂₆N₄ = 586.7) |
| 2-12 | m/z = 640.2(C₄₆H₂₈N₂S = 640.8) |
| 2-13 | m/z = 466.18(C₃₂H₁₄D₅N₃O = 466.55) |
| 2-14 | m/z = 715.27(C₅₁H₃₃N₅ = 715.86) |
| 2-15 | m/z = 491.15(C₃₃H₂₁N₃S = 491.61) |
| 2-16 | m/z = 590.2(C₄₂H₂₆N₂O₂ = 590.68) |
| 2-17 | m/z = 458.18(C₃₄H₂₂N₂ = 458.56) |
| 2-18 | m/z = 449.12(C₃₂H₁₉NS = 449.57) |
| 2-19 | m/z = 433.15(C₃₂H₁₉NO = 433.51) |
| 2-20 | m/z = 399.11(C₂₈H₁₇NS = 399.51) |
| 2-21 | m/z = 527.15(C₃₆H₂₁N₃S = 527.65) |
| 2-22 | m/z = 583.12(C₃₈H₂₁N₃S₂ = 583.73) |
| 2-23 | m/z = 577.16(C₄₀H₂₃N₃S = 577.71) |
| 2-24 | m/z = 527.15(C₃₆H₂₁N₃S = 527.65) |
| 2-25 | m/z = 475.14(C₃₄H₂₁NS = 475.61) |
| 2-26 | m/z = 585.21(C₄₄H₂₇NO = 585.71) |
| 2-27 | m/z = 509.19(C₃₇H₂₃N₃ = 509.61) |
| 2-28 | m/z = 451.11(C₃₀H₁₇N₃S = 451.55) |
| 2-29 | m/z = 588.2(C₄₁H₂₄N₄O = 588.67) |
| 2-30 | m/z = 614.18(C₄₄H₂₆N₂S = 614.77) |
| 2-31 | m/z = 449.12(C₃₂H₁₉NS = 449.57) |
| 2-32 | m/z = 573.17(C₄₂H₂₃NO₂ = 573.65) |
| 2-33 | m/z = 624.26(C₄₇H₃₂N₂ = 624.79) |
| 2-34 | m/z = 603.18(C₄₂H₂₅N₃S = 603.74) |
| 2-35 | m/z = 664.23(C₄₇H₂₈N₄O = 664.77) |
| 2-36 | m/z = 738.28(C₅₄H₃₄N₄ = 738.89) |
| 2-37 | m/z = 679.21(C₄H₂₉N₃S = 679.84) |
| 2-38 | m/z = 625.22(C₄₅H₂7N₃O = 625.73) |
| 2-39 | m/z = 508.19(C₃₈H₂₄N₂ = 508.62) |
| 2-40 | m/z = 449.12(C₃₂H₁₉NS = 449.57) |
| 2-41 | m/z = 433.15(C₃₂H₁₉NO = 433.51) |
| 2-42 | m/z = 608.23(C₄₆H₂₈N₂ = 608.74) |
| 2-43 | m/z = 577.16(C₄₀H₂₃N₃S = 577.71) |
| 2-44 | m/z = 384.13(C₂₇H₁₆N₂O = 384.44) |
| 2-45 | m/z = 508.19(C₃₈H₂₄N₂ = 508.62) |
| 2-46 | m/z = 449.12(C₃₂H₁₉NS = 449.57) |
| 2-47 | m/z = 433.15(C₃₂H₁₉NO = 433.51) |
| 2-48 | m/z = 663.24(C₄₇H₂₉N₅ = 663.78) |
| 2-49 | m/z = 603.18(C₄₂H₂₅N₃S = 603.74) |
| 2-50 | m/z = 587.2(C₄₂H₂₅N₃O = 587.68) |
| 2-51 | m/z = 662.25(C₄₈H₃₀N₄ = 662.8) |
| 2-52 | m/z = 577.16(C₄₀H₂₃N₃S = 577.71) |
| 2-53 | m/z = 601.18(C₄₂H₂₃N₃O₂ = 601.67) |
| 2-54 | m/z = 586.22(C₄₂H₂₆N₄ = 586.7) |
| 2-55 | m/z = 630.19(C₄₃H₂₆N₄S = 630.77) |
| 2-56 | m/z = 613.22(C₄₄H₂₇N₃O = 613.72) |
| 2-57 | m/z = 508.19(C₃₈H₂₄N₂ = 508.62) |
| 2-58 | m/z = 449.12(C₃₂H₁₉NS = 449.57) |
| 2-59 | m/z = 433.15(C₃₂H₁₉NO = 433.51) |
| 2-60 | m/z = 663.24(C₄₇H₂₉N₅ = 663.78) |
| 2-61 | m/z = 627.18(C₄₄H₂₅N₃S = 627.77) |
| 2-62 | m/z = 587.2(C₄₂H₂₅N₃O = 587.68) |
| 2-63 | m/z = 527.15(C₃₆H₂₁N₃S = 527.65) |
| 2-64 | m/z = 603.18(C₄₂H₂₅N₃S = 603.74) |
| 2-65 | m/z = 516.2(C₃₆H₁₆D₅N₃O = 516.61) |
| 2-66 | m/z = 692.2(C₄₈H₂₈N₄S = 692.84) |
| 2-67 | m/z = 577.16(C₄₀H₂₃N₃S = 577.71) |
| 2-68 | m/z = 561.18(C₄₀H₂₃N₃0 = 561.64) |
| 2-69 | m/z = 653.19(C₄₆H₂₇N₃S = 653.8) |
| 2-70 | m/z = 736.26(C₅₄H₃₂N₄ = 736.88) |
| 2-71 | m/z = 677.19(C₄₈H₂₇N₃S = 677.83) |
| 2-72 | m/z = 692.26(C₅₀H₂₄D₅N₃O = 692.83) |
| 2-73 | m/z = 703.21(C₅₀H₂₉N₃S = 703.86) |
| 2-74 | m/z = 603.18(C₄₂H₂₅N₃S = 603.74) |
| 2-75 | m/z = 659.15(C₄₄H₂₅N₃S₂ = 659.83) |
| 2-76 | m/z = 759.18(C₅₂H₂₉N₃S₂ = 759.95) |
| 2-77 | m/z = 475.14(C₃₄H₂₁NS = 475.61) |
| 2-78 | m/z = 616.2(C₄₄H₂₈N₂S = 616.78) |
| 2-79 | m/z = 728.15(C₄₇H₂₅FN₄S₂ = 728.86) |
| 2-80 | m/z = 818.25(C₅₈H₃₄N₄S = 819) |
| 2-81 | m/z = 552.17(C₃₉H₂₄N₂S = 552.7) |
| 2-82 | m/z = 809.2(C₅₆H₃₁N₃S₂ = 810.01) |
| 2-83 | m/z = 630.19(C₄₃H₂₆N₄S = 630.77) |
| 2-84 | m/z = 723.27(C₅₁H₃₇N₃S = 723.94) |
| 2-85 | m/z = 844.27(C₆₀H₃₆N₄S = 845.04) |
| 2-86 | m/z = 706.22(C₄₉H₃₀N₄S = 706.87) |
| 2-87 | m/z = 728.22(C₅₁H₂₈N₄O₂ = 728.81) |
| 2-88 | m/z = 759.25(C₅₃H₃₃N₃O₃ = 759.87) |
| 2-89 | m/z = 677.19(C₄₈H₂₇N₃S = 677.83) |
| 2-90 | m/z = 683.15(C₄₆H₂₅N₃S₂ = 683.85) |
| 2-91 | m/z = 668.17(C₄₅H₂₄N₄OS = 668.77) |
| 2-92 | m/z = 653.19(C₄₆H₂₇N₃S = 653.8) |
| 2-93 | m/z = 604.17(C₄₁H₂₄N₄S = 604.73) |
| 2-94 | m/z = 878.3(C₆₄H₃₈N₄O = 879.04) |
| 2-95 | m/z = 588.2(C₄₁H₂₄N₄O = 588.67) |
| 2-96 | m/z = 561.18(C₄₀H₂₃N₃0 = 561.64) |
| 2-97 | m/z = 601.18(C₄₂H₂₃N₃O₂ = 601.67) |
| 2-98 | m/z = 664.23(C₄₇H₂₈N₄O = 664.77) |
| 3-1 | m/z = 408.16(C₃₀H₂₀N₂ = 408.5) |
| 3-2 | m/z = 536.2(C₃₈H₂₄N₄ = 536.64) |
| 3-3 | m/z = 449.12(C₃₂H₁₉NS = 449.57) |
| 3-4 | m/z = 461.15(C₃₂H₁₉N₃O = 461.52) |
| 3-5 | m/z = 636.23(C₄₆H₂₈N₄ = 636.76) |
| 3-6 | m/z = 457.07(C₂₈H₁₅N₃S₂ = 457.57) |
| 3-7 | m/z = 593.16(C₄₀H₂₃N₃OS = 593.7) |
| 3-8 | m/z = 524.23(C₃₉H₂₈N₂ = 524.67) |
| 3-9 | m/z = 554.16(C₃₇H₂₂N₄S = 554.67) |
| 3-10 | m/z = 693.19(C₄₈H₂₇N₃OS = 693.82) |
| 3-11 | m/z = 586.22(C₄₂H₂₆N₄ = 586.7) |
| 3-12 | m/z = 640.2(C₄₆H₂₈N₂S = 640.8) |
| 3-13 | m/z = 466.18(C₃₂H₁₄D₅N₃O = 466.55) |
| 3-14 | m/z = 715.27(C₅₁H₃₃N₅ = 71 5.86) |
| 3-15 | m/z = 491.15(C₃₃H₂₁N₃S = 491.61) |
| 3-16 | m/z = 590.2(C₄₂H₂₆N₂O₂ = 590.68) |
| 3-17 | m/z = 458.18(C₃₄H₂₂N₂ = 458.56) |
| 3-18 | m/z = 449.12(C₃₂H₁₉NS = 449.57) |
| 3-19 | m/z = 433.15(C₃₂H₁₉NO = 433.51) |
| 3-20 | m/z = 399.11(C₂₈H₁₇NS = 399.51) |
| 3-21 | m/z = 527.15(C₃₆H₂₁N₃S = 527.65) |
| 3-22 | m/z = 583.12(C₃₈H₂₁N₃S₂ = 583.73) |
| 3-23 | m/z = 577.16(C₄₀H₂₃N₃S = 577.71) |
| 3-24 | m/z = 627.18(C₄₄H₂₅N₃S = 627.77) |
| 3-25 | m/z = 554.16(C₃₇H₂₂N₄S = 554.67) |
| 3-26 | m/z = 585.21(C₄₄H₂₇NO = 585.71) |
| 3-27 | m/z = 509.19(C₃₇H₂₃N₃ = 509.61) |
| 3-28 | m/z = 451.11(C₃₀H₁₇N₃S = 451.55) |
| 3-29 | m/z = 588.2(C₄₁H₂₄N₄O = 588.67) |
| 3-30 | m/z = 614.18(C₄₄H₂₆N₂S = 61 4.77) |
| 3-31 | m/z = 449.12(C₃₂H₁₉NS = 449.57) |
| 3-32 | m/z = 573.17(C₄₂H₂₃NO₂ = 573.65) |
| 3-33 | m/z = 624.26(C₄₇H₃₂N₂ = 624.79) |
| 3-34 | m/z = 527.15(C₃₆H₂₁N₃S = 527.65) |
| 3-35 | m/z = 664.23(C₄₇H₂₈N₄O = 664.77) |
| 3-36 | m/z = 738.28(C₅₄H₃₄N₄ = 738.89) |
| 3-37 | m/z = 679.21(C₄₈H₂₉N₃S = 679.84) |
| 3-38 | m/z = 625.22(C₄₅H₂₇N₃O = 625.73) |
| 3-39 | m/z = 508.19(C₃₈H₂₄N₂ = 508.62) |
| 3-40 | m/z = 449.12(C₃₂H₁₉NS = 449.57) |
| 3-41 | m/z = 433.15(C₃₂H₁₉NO = 433.51) |
| 3-42 | m/z = 608.23(C₄₆H₂₈N₂ = 608.74) |
| 3-43 | m/z = 475.14(C₃₄H₂₁NS = 475.61) |
| 3-44 | m/z = 511.17(C₃₆H₂₁N₃O = 511.58) |
| 3-45 | m/z = 508.19(C₃₈H₂₄N₂ = 508.62) |
| 3-46 | m/z = 449.12(C₃₂H₁₉NS = 449.57) |
| 3-47 | m/z = 433.15(C₃₂H₁₉NO = 433.51) |
| 3-48 | m/z = 663.24(C₄₇H₂₉N₅ = 663.78) |
| 3-49 | m/z = 604.17(C₄₁H₂₄N₄S = 604.73) |
| 3-50 | m/z = 587.2(C₄₂H₂₅N₃O = 587.68) |
| 3-51 | m/z = 662.25(C₄₈H₃₀N₄ = 662.8) |
| 3-52 | m/z = 577.16(C₄₀H₂₃N₃S = 577.71) |
| 3-53 | m/z = 601.18(C₄₂H₂₃N₃O₂ = 601.67) |
| 3-54 | m/z = 586.22(C₄₂H₂₆N₄ = 586.7) |
| 3-55 | m/z = 577.16(C₄₀H₂₃N₃S = 577.71) |
| 3-56 | m/z = 617.16(C₄₂H₂₃N₃OS = 61 7.73) |
| 3-57 | m/z = 508.19(C₃₈H₂₄N₂ = 508.62) |
| 3-58 | m/z = 449.12(C₃₂H₁₉NS = 449.57) |
| 3-59 | m/z = 433.15(C₃₂H₁₉NO = 433.51) |
| 3-60 | m/z = 663.24(C₄₇H₂₉N₅ = 663.78) |
| 3-61 | m/z = 604.17(C₄₁H₂₄N₄S = 604.73) |
| 3-62 | m/z = 587.2(C₄₂H₂₅N₃O = 587.68) |
| 3-63 | m/z = 527.15(C₃₆H₂₁N₃S = 527.65) |
| 3-64 | m/z = 603.18(C₄₂H₂₅N₃S = 603.74) |
| 3-65 | m/z = 516.2(C₃₆H₁₆D₅N₃O = 516.61) |
| 3-66 | m/z = 692.2(C₄₈H₂₈N₄S = 692.84) |
| 3-67 | m/z = 577.16(C₄₀H₂₃N₃S = 577.71) |
| 3-68 | m/z = 561.18(C₄₀H₂₃N₃0 = 561.64) |
| 3-69 | m/z = 653.19(C₄₆H₂₇N₃S = 653.8) |
| 3-70 | m/z = 736.26(C₅₄H₃₂N₄ = 736.88) |
| 3-71 | m/z = 677.19(C₄₈H₂₇N₃S = 677.83) |
| 3-72 | m/z = 692.26(C₅₀H₂₄D₅N₃O = 692.83) |
| 3-73 | m/z = 703.21(C₅₀H₂₉N₃S = 703.86) |
| 3-74 | m/z = 603.18(C₄₂H₂₅N₃S = 603.74) |
| 3-75 | m/z = 735.1 8(C₅₀H₂₉N₃S₂ = 735.92) |
| 3-76 | m/z = 784.18(C₅₃H₂₈N₄S₂ = 784.96) |
| 3-77 | m/z = 475.14(C₃₄H₂₁NS = 475.61) |
| 3-78 | m/z = 616.2(C₄₄H₂₈N₂S = 616.78) |
| 3-79 | m/z = 710.16(C₄₇H₂₆N₄S₂ = 710.87) |
| 3-80 | m/z = 818.25(C₅₈H₃₄N₄S = 819) |
| 3-81 | m/z = 603.18(C₄₂H₂₅N₃S = 603.74) |
| 3-82 | m/z = 809.2(C₅₆H₃₁N₃S₂ = 810.01) |
| 3-83 | m/z = 659.15(C₄₄H₂₅N₃S₂ = 659.83) |
| 3-84 | m/z = 623.24(C₄₃H₃₃N₃S = 623.82) |
| 3-85 | m/z = 844.27(C₆₀H₃₆N₄S = 845.04) |
| 3-86 | m/z = 667.17(C₄₆H₂₅N₃OS = 667.79) |
| 3-87 | m/z = 703.23(C₅₀H₂₉N₃0₂ = 703.8) |
| 3-88 | m/z = 659.22(C₄₅H₂₉N₃O₃ = 659.75) |
| 3-89 | m/z = 780.29(C₅₆H₃₆N₄O = 780.93) |
| 3-90 | m/z = 600.22(C₄₄H₂₈N₂O = 600.72) |
| 3-91 | m/z = 858.25(C₆₀H₃₄N₄OS = 859.02) |
| 3-92 | m/z = 792.26(C₅₅H₃₂N₆O = 792.9) |
| 3-93 | m/z = 794.34(C₅₈H₄₂N₄ = 795) |
| 3-94 | m/z = 701.28(C₅₂H₃₅N₃ = 701.87) |
| 3-95 | m/z = 867.3(C₆₂H₃₇N₅O = 868.01) |
| 3-96 | m/z = 764.29(C₅₆H₃₆N₄ = 764.93) |
| 3-97 | m/z = 690.21(C₅₀H₃₀N₂S = 690.86) |
| 3-98 | m/z = 718.24(C₅₂H₃₄N₂S = 718.92) |
| 3-99 | m/z = 894.28(C₆₄H₃₈N₄S = 895.1) |
| 3-100 | m/z = 554.16(C₃₇H₂₂N₄S = 554.67) |
| 3-101 | m/z = 554.16(C₃₇H₂₂N₄S = 554.67) |
| 3-102 | m/z = 511.17(C₃₆H₂₁N₃O = 511.58) |
| 3-103 | m/z = 538.18(C₃₇H₂₂N₄O = 538.61) |
| 3-104 | m/z = 588.2(C₄₁H₂₄N₄O = 588.67) |
| 4-1 | m/z = 458.18(C₃₄H₂₂N₂ = 458.56) |
| 4-2 | m/z = 449.12(C₃₂H₁₉NS = 449.57) |
| 4-3 | m/z = 433.15(C₃₂H₁₉NO = 433.51) |
| 4-4 | m/z = 399.11(C₂₈H₁₇NS = 399.51) |
| 4-5 | m/z = 527.15(C₃₆H₂₁N₃S = 527.65) |
| 4-6 | m/z = 583.12(C₃₈H₂₁N₃S₂ = 583.73) |
| 4-7 | m/z = 577.16(C₄₀H₂₃N₃S = 577.71) |
| 4-8 | m/z = 627.18(C₄₄H₂₅N₃S = 627.77) |
| 4-9 | m/z = 475.14(C₃₄H₂₁NS = 475.61) |
| 4-10 | m/z = 585.21(C₄₄H₂₇NO = 585.71) |
| 4-11 | m/z = 509.19(C₃₇H₂₃N₃ = 509.61) |
| 4-12 | m/z = 451.11(C₃₀H₁₇N₃S = 451.55) |
| 4-13 | m/z = 588.2(C₄₁H₂₄N₄O = 588.67) |
| 4-14 | m/z = 614.18(C₄₄H₂₆N₂S = 614.77) |
| 4-15 | m/z = 449.12(C₃₂H₁₉NS = 449.57) |
| 4-16 | m/z = 573.17(C₄₂H₂₃NO₂ = 573.65) |
| 4-17 | m/z = 624.26(C₄₇H₃₂N₂ = 624.79) |
| 4-18 | m/z = 603.18(C₄₂H₂₅N₃S = 603.74) |
| 4-19 | m/z = 664.23(C₄₇H₂₈N₄O = 664.77) |
| 4-20 | m/z = 738.28(C₅₄H₃₄N₄ = 738.89) |
| 4-21 | m/z = 679.21(C₄₈H₂₉N₃S = 679.84) |
| 4-22 | m/z = 625.22(C₄₅H₂₇N₃O = 625.73) |
| 4-23 | m/z = 508.19(C₃₈H₂₄N₂ = 508.62) |
| 4-24 | m/z = 449.12(C₃₂H₁₉NS = 449.57) |
| 4-25 | m/z = 433.15(C₃₂H₁₉NO = 433.51) |
| 4-26 | m/z = 608.23(C₄₆H₂₈N₂ = 608.74) |
| 4-27 | m/z = 475.14(C₃₄H₂₁NS = 475.61) |
| 4-28 | m/z = 384.13(C₂₇H₁₆N₂O = 384.44) |
| 4-29 | m/z = 508.19(C₃₈H₂₄N₂ = 508.62) |
| 4-30 | m/z = 449.12(C₃₂H₁₉NS = 449.57) |
| 4-31 | m/z = 433.15(C₃₂H₁₉NO = 433.51) |
| 4-32 | m/z = 663.24(C₄₇H₂₉N₅ = 663.78) |
| 4-33 | m/z = 603.18(C₄₂H₂₅N₃S = 603.74) |
| 4-34 | m/z = 587.2(C₄₂H₂₅N₃O = 587.68) |
| 4-35 | m/z = 662.25(C₄₈H₃₀N₄ = 662.8) |
| 4-36 | m/z = 577.16(C₄₀H₂₃N₃S = 577.71) |
| 4-37 | m/z = 601.18(C₄₂H₂₃N₃O₂ = 601.67) |
| 4-38 | m/z = 586.22(C₄₂H₂₆N₄ = 586.7) |
| 4-39 | m/z = 630.19(C₄₃H₂₆N₄S = 630.77) |
| 4-40 | m/z = 613.22(C₄₄H₂₇N₃O = 613.72) |
| 4-41 | m/z = 508.19(C₃₈H₂₄N₂ = 508.62) |
| 4-42 | m/z = 449.12(C₃₂H₁₉NS = 449.57) |
| 4-43 | m/z = 433.15(C₃₂H₁₉NO = 433.51) |
| 4-44 | m/z = 663.24(C₄₇H₂₉N₅ = 663.78) |
| 4-45 | m/z = 604.17(C₄₁H₂₄N₄S = 604.73) |
| 4-46 | m/z = 587.2(C₄₂H₂₅N₃O = 587.68) |
| 4-47 | m/z = 527.15(C₃₆H₂₁N₃S = 527.65) |
| 4-48 | m/z = 603.18(C₄₂H₂₅N₃S = 603.74) |
| 4-49 | m/z = 516.2(C₃₆H₁₆D₅N₃O = 516.61) |
| 4-50 | m/z = 692.2(C₄₈H₂₈N₄S = 692.84) |
| 4-51 | m/z = 577.16(C₄₀H₂₃N₃S = 577.71) |
| 4-52 | m/z = 561.18(C₄₀H₂₃N₃0 = 561.64) |
| 4-53 | m/z = 653.19(C₄₆H₂₇N₃S = 653.8) |
| 4-54 | m/z = 736.26(C₅₄H₃₂N₄ = 736.88) |
| 4-55 | m/z = 677.19(C₄₈H₂₇N₃S = 677.83) |
| 4-56 | m/z = 692.26(C₅₀H₂₄D₅N₃O = 692.83) |
| 4-57 | m/z = 703.21(C₅₀H₂₉N₃S = 703.86) |
| 4-58 | m/z = 605.19(C₄₂H₂₇N₃S = 605.76) |
| 4-59 | m/z = 735.18(C₅₀H₂₉N₃S₂ = 735.92) |
| 4-60 | m/z = 759.18(C₅₂H₂₉N₃S₂ = 759.95) |
| 4-61 | m/z = 475.14(C₃₄H₂₁NS = 475.61) |
| 4-62 | m/z = 616.2(C₄₄H₂₈N₂S = 616.78) |
| 4-63 | m/z = 728.15(C₄₇H₂₅FN₄S₂ = 728.86) |
| 4-64 | m/z = 818.25(C₅₈H₃₄N₄S = 819) |
| 4-65 | m/z = 603.18(C₄₂H₂₅N₃S = 603.74) |
| 4-66 | m/z = 809.2(C₅₆H₃₁N₃S₂ = 810.01) |
| 4-67 | m/z = 659.15(C₄₄H₂₅N₃S₂ = 659.83) |
| 4-68 | m/z = 623.24(C₄₃H₃₃N₃S = 623.82) |
| 4-69 | m/z = 844.27(C₆₀H₃₆N₄S = 845.04) |
| 4-70 | m/z = 667.17(C₄₆H₂₅N₃OS = 667.79) |
| 4-71 | m/z = 703.23(C₅₀H₂₉N₃O₂ = 703.8) |
| 4-72 | m/z = 659.22(C₄₅H₂₉N₃O₃ = 659.75) |
| 4-73 | m/z = 828.29(C₆₀H₃₆N₄O = 828.98) |
| 4-74 | m/z = 617.16(C₄₂H₂₃N₃OS = 617.73) |
| 4-75 | m/z = 858.25(C₆₀H₃₄N₄OS = 859.02) |
| 4-76 | m/z = 792.26(C₅₅H₃₂N₆O = 792.9) |
| 4-77 | m/z = 794.34(C₅₈H₄₂N₄ = 795) |
| 4-78 | m/z = 878.3(C₆₄H₃₈N₄O = 879.04) |
| 4-79 | m/z = 638.21(C₄₅H₂₆N₄O = 638.73) |
| 4-80 | m/z = 511.17(C₃₆H₂₁N₃O = 511.58) |
| 4-81 | m/z = 601.18(C₄₂H₂₃N₃O2 = 601.67) |
| 4-82 | m/z = 740.26(C₅₃H₃₂N₄O = 740.87) |
| 4-83 | m/z = 639.24(C₄₅H₂₉N₅ = 639.76) |
| 4-84 | m/z = 536.2(C₃₈H₂₄N₄ = 536.64) |
| 4-85 | m/z = 439.1(C₃₀H₁₇NOS = 439.53) |
| 4-86 | m/z = 475.14(C₃₄H₂₁NS = 475.61) |
| 4-87 | m/z = 333.12(C₂₄H₁₅NO = 333.39) |
| 4-88 | m/z = 564.2(C₃₉H₂₄N₄O = 564.65) |
| 4-89 | m/z = 649.25(C₄₈H₃₁N₃ = 649.8) |
| 4-90 | m/z = 574.2(C₄₂H₂₆N₂O = 574.68) |
| 4-91 | m/z = 485.18(C₃₆H₂₃NO = 485.59) |
| 4-92 | m/z = 663.23(C₄₈H₂₉N₃O = 663.78) |
| 4-93 | m/z = 541.19(C₃₉H₂₇NS = 541.71) |
| 4-94 | m/z = 578.18(C₄₁H₂₆N₂S = 578.73) |

### [Example 1] Red Organic Light Emitting Diode

First, on an ITO layer (anode) formed on a glass substrate, N1-(naphthalen-2-yl)-N4,N4-bis(4-(naphthalen-2-yl(phenyl)amino)phenyl)-N1-phenylbenzene-1,4-diamine (hereinafter, abbreviated as 2-TNANA) film as a hole injection layer was vacuum-deposited to form a thickness of 60 nm. Subsequently, 4,4-bis[N-(1-naphthyl)-N-phenylamino]biphenyl (hereinafter abbreviated as—NPD) as a hole transport compound was vacuum-deposited on the film to a thickness of 60 nm to form a hole transport layer. Then, the compound (1-11) of the present invention was vacuum-deposited to a thickness of 30 nm as an emitting auxiliary layer material to form an emitting auxiliary layer. Then, on the emitting auxiliary layer, an emitting layer with a thickness of 30 nm was deposited by doping the compound 2-14 of the present invention as a host and (piq)2Ir(acac)[bis-(1-phenylisoquinolyl)iridium(III)acetylacetonate] as a dopant at a weight of 95:5. Thereafter, on the emitting layer, (1,1′bisphenyl)-4-oleato)bis(2-methyl-8-quinolineoleato)aluminum (hereinafter, abbreviated as BAlq) is vacuum-deposited to a thickness of 10 nm to form a hole blocking layer, and Tris(8-quinolinol)aluminum (hereinafter, abbreviated as Alq3) was deposited on the hole blocking layer to a thickness of 40 nm to form an electron transport layer. Thereafter, on the electron transport layer, LiF, which is an alkali metal halide, is deposited to a thickness of 0.2 nm as an electron injection layer, then Al was deposited to a thickness of 150 nm and used as a cathode to prepare an organic electroluminescent device.

### [Example 2] to [Example 20]

An organic electroluminescent device was manufactured in the same manner as in Example 1, except that the compounds of the present invention shown in Table 7 were used for the emitting auxiliary layer and the emitting layer.

### Example 21

An organic electroluminescent device was manufactured in the same manner as in Example 1, except that the compounds of the present invention were used in the hole transport layer, the emitting auxiliary layer and the emitting layer as shown in Table 7.

### Comparative Example 1

An organic electroluminescent device was manufactured in the same manner as in Example 1, except that the emitting auxiliary layer was not used.

### [Comparative Example 2] to [Comparative Example 6]

An organic electroluminescent device was manufactured in the same manner as in Example 1, except that an emitting auxiliary layer material and a host material were used as shown in Table 7.

### <Comparative Example 1> <Comparative Example 2> <Comparative Example 3> <Comparative Example 4>

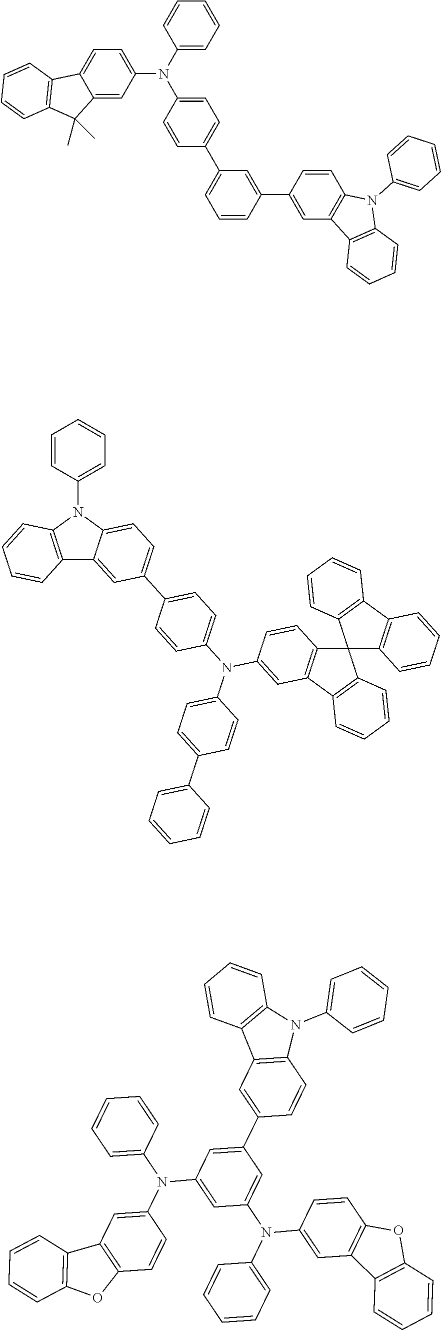 [SMILES: CC1(C)c2ccccc2-c2ccc(N(c3ccccc3)c3ccc(-c4cccc(-c5ccc6c(c5)c5ccccc5n6-c5ccccc5)c4)cc3)cc21 ; c1ccc(-c2ccc(N(c3ccc(-c4ccc5c(c4)c4ccccc4n5-c4ccccc4)cc3)c3ccc4c(c3)-c3ccccc3C43c4ccccc4-c4ccccc43)cc2)cc1 ; c1ccc(N(c2cc(-c3ccc4c(c3)c3ccccc3n4-c3ccccc3)cc(N(c3ccccc3)c3ccc4oc5ccccc5c4c3)c2)c2ccc3oc4ccccc4c3c2)cc1]

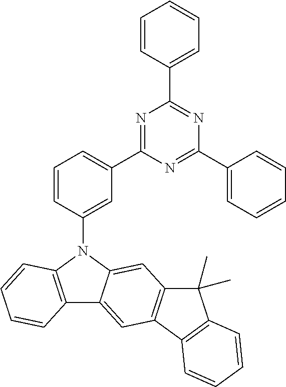 [SMILES: CC1(C)c2ccccc2-c2cc3c4ccccc4n(-c4cccc(-c5nc(-c6ccccc6)nc(-c6ccccc6)n5)c4)c3cc21]

By applying a forward bias DC voltage to the organic electroluminescent devices prepared by Example 1 to Example 21 and Comparative Example 1 to Comparative Example 6 of the present invention, Electroluminescence (EL) characteristics were measured with PR-650 from Photoresearch, and the T95 lifetime was measured using a lifetime measuring device manufactured by McScience at 2500 cd/m² standard luminance. The measurement results are shown in Table 7.

TABLE 7

| | Hole | Emitting | | | Current | | | |
|---|---|---|---|---|---|---|---|---|
| | transport | auxiliary | Emitting | | Density | Brightness | Efficiency | |
| | layer | layer | layer | Voltage | (mA/cm²) | (cd/m²) | (cd/A) | T(95) |
| comparative | NPD | — | comparative | 6.8 | 29.4 | 2500.0 | 8.5 | 62.4 |
| example(1) | | | compound4 | | | | | |
| comparative | NPD | comparative | comparative | 6.6 | 16.1 | 2500.0 | 15.5 | 64.9 |
| example(2) | | compound1 | compound4 | | | | | |
| comparative | NPD | comparative | comparative | 6.5 | 15.0 | 2500.0 | 16.7 | 71.1 |
| example(3) | | compound2 | compound4 | | | | | |
| comparative | NPD | comparative | comparative | 6.3 | 20.2 | 2500.0 | 12.4 | 78.5 |
| example(4) | | compound3 | compound4 | | | | | |
| comparative | NPD | comparative | 2-14 | 6.3 | 11.2 | 2500.0 | 22.4 | 91.1 |
| example(5) | | compound2 | | | | | | |
| comparative | NPD | 1-2 | comparative | 6.2 | 10.0 | 2500.0 | 25.1 | 88.7 |
| example(6) | | | compound4 | | | | | |
| example(1) | NPD | 1-11 | 2-14 | 5.6 | 7.4 | 2500.0 | 33.8 | 116.0 |
| example(2) | NPD | 1-11 | 2-74 | 5.6 | 7.7 | 2500.0 | 32.5 | 115.9 |
| example(3) | NPD | 1-11 | 2-95 | 5.9 | 8.0 | 2500.0 | 31.3 | 115.8 |
| example(4) | NPD | 1-11 | 3-102 | 6.0 | 9.2 | 2500.0 | 27.2 | 110.6 |
| example(5) | NPD | 1-11 | 4-54 | 6.0 | 9.0 | 2500.0 | 27.8 | 112.1 |
| example(6) | NPD | 1-39 | 2-14 | 5.6 | 7.5 | 2500.0 | 33.5 | 113.8 |
| example(7) | NPD | 1-39 | 2-74 | 5.6 | 7.1 | 2500.0 | 35.1 | 117.8 |
| example(8) | NPD | 1-39 | 2-95 | 5.8 | 7.8 | 2500.0 | 31.9 | 112.9 |
| example(9) | NPD | 1-39 | 3-102 | 5.8 | 8.7 | 2500.0 | 28.6 | 109.5 |
| example(10) | NPD | 1-39 | 4-54 | 5.9 | 8.5 | 2500.0 | 29.3 | 111.8 |
| example(11) | NPD | 1-46 | 2-14 | 5.7 | 7.8 | 2500.0 | 31.9 | 112.6 |
| example(12) | NPD | 1-46 | 2-74 | 5.7 | 7.8 | 2500.0 | 32.1 | 110.8 |
| example(13) | NPD | 1-46 | 2-95 | 5.8 | 7.9 | 2500.0 | 31.5 | 109.7 |
| example(14) | NPD | 1-46 | 3-102 | 5.9 | 8.8 | 2500.0 | 28.3 | 108.5 |
| example(15) | NPD | 1-46 | 4-54 | 6.0 | 8.8 | 2500.0 | 28.4 | 106.4 |
| example(16) | NPD | 1-141 | 2-14 | 5.8 | 7.9 | 2500.0 | 31.6 | 112.0 |
| example(17) | NPD | 1-141 | 2-74 | 5.8 | 8.2 | 2500.0 | 30.6 | 110.0 |
| example(18) | NPD | 1-141 | 2-95 | 5.9 | 9.2 | 2500.0 | 27.2 | 105.9 |
| example(19) | NPD | 1-141 | 3-102 | 6.0 | 9.6 | 2500.0 | 26.1 | 99.4 |
| example(20) | NPD | 1-141 | 4-54 | 6.1 | 9.6 | 2500.0 | 26.0 | 101.7 |
| example(21) | 1-54 | 1-16 | 3-2 | 5.7 | 7.1 | 2500.0 | 35.1 | 115.8 |

From the results of Table 7, it can be seen that the driving voltage of Examples 1 to 20 using the compound of the present invention represented by Formula 1 as an emitting auxiliary layer material and using the compound of the present invention represented by Formula 2 as an emitting layer material is lowered and the efficiency and lifespan are significantly improved. In the case of Comparative Examples 2 to 4 using one of Comparative Compounds 1 to 3 in the emitting auxiliary layer than Comparative Example 1 using Comparative Compound 4 as a host without forming an emitting auxiliary layer, the driving voltage of the element is lowered, and the efficiency and lifespan are improved. Also, the element performance of Comparative Examples 5 and 6 was superior to that of Comparative Examples 2 to 4, and when an emitting auxiliary layer is formed with the compound represented by Formula 1 of the present invention and the compound represented by Formula 2 is used as a host, the driving voltage, efficiency, and lifespan of the element are significantly improved compared to Comparative Examples 1 to 6.

It is thought that this is because the compounds of the present invention represented by Formula 1 have a deep HOMO energy level, when used as an emitting auxiliary layer, holes and electrons achieve charge balance, and light is emitted inside the emitting layer, not at the hole transport layer interface, thereby maximizing the efficiency. Also, by using the compound of the present invention represented by Formula 2 as a phosphorescent host, it is determined that the combination of this element has a synergistic effect electrochemically to improve the overall element performance.

Furthermore, in the evaluation result of the above-described device fabrication, the device characteristics in which the compound represented by Formula 1 was applied to the emitting auxiliary layer were described, but as in the device result of Example 21, excellent performance was also shown when applied to one or more of the hole transport layer and the emitting auxiliary layer.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device having excellent device characteristics of high luminance, high light emission and long lifespan, and thus there is industrial applicability.

What is claimed is:

1. An organic electronic element comprising an anode, a cathode, and an organic material layer formed between the anode and the cathode, wherein the organic material layer comprises an emitting layer, and a hole transport band layer formed between the emitting layer and the anode, wherein the hole transport band layer comprises a compound represented by Formula 1, and the emitting layer comprises a compound represented by Formula 2: 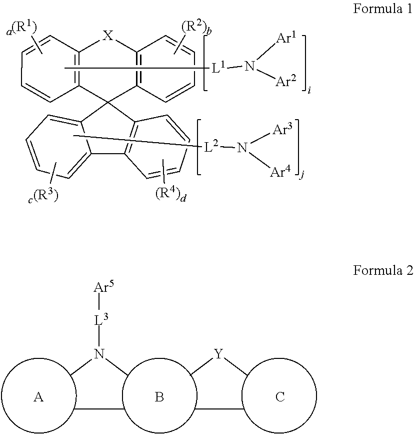
wherein:
1) X is O, S or NR⁵,
2) Y is O, S or NR⁶,
3) Ring A, ring B and ring C are each independently a C₆-C₁₄ aryl group, and Ring A may be substituted with R⁷, Ring B may be substituted with R⁸, and Ring C may be substituted with R⁹,
4) R¹, R², R³, R⁴, R⁷, R⁸ and R⁹ are the same as or different from each other, and are each independently selected from the group consisting of hydrogen; deuterium; halogen; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; a C₆-C₆₀ aryl group; fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; and C₆-C₆₀ arylamine group; or in case a, b, c and d are 2 or more, a plurality of adjacent R¹s, or a plurality of R²s, or a plurality of R³s, or a plurality of R⁴s may be bonded to each other to form a ring,
5) R⁵ is an C₆˜C₆₀ aryl group; or a C₂-C₆₀ heterocyclic group including at least one heteroatom of O, N, S, Si or P; and R⁶ is an C₆˜C₆₀ aryl group; or a C₂-C₆₀ heterocyclic group including at least one heteroatom of O, N, S, Si or P; or L-Ar, wherein L is the same as L¹, Ar is the same as Ar¹,
6) a, b, c and d are each independently an integer of 0 to 4,
7) i and j are each independently an integer of 0 to 2, provided that i+j is an integer of 1 or more,
8) L¹, L² and L³ are each independently selected from the group consisting of a single bond; a C₆-C₆₀ arylene group; fluorenylene group; a fused ring group of a C₃-C₆₀ aliphatic ring and a C₆-C₆₀ aromatic ring; and a C₂-C₆₀ heterocyclic group;
9) Ar¹, Ar², Ar³, Ar⁴ and Ar⁵ are each independently selected from the group consisting of a C₁-C₆₀ alkyl group; a C₂-C₂₀ alkenyl group; a C₂-C₂₀ alkynyl group; a C₁-C₃₀ alkoxyl group; a C₆-C₃₀ aryloxy group; a C₆-C₆₀ aryl group; fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring; or Ar¹ and Ar² or Ar³ and Ar⁴ may be bonded to each other to form a ring,
10) wherein the aryl group, arylene group, arylamine group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀cycloalkyl group; C₇-C₂₀ arylalkyl group; and C₈-C₂₀ arylalkenyl group; and the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

2. The organic electronic element of claim 1, wherein the compound represented by Formula 1 is represented by any one of Formulas 1-1 to 1-7: 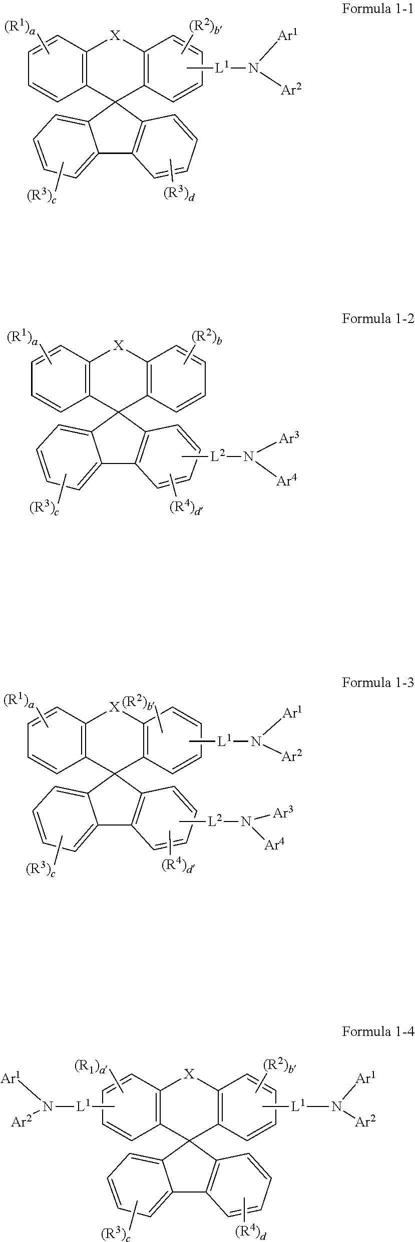 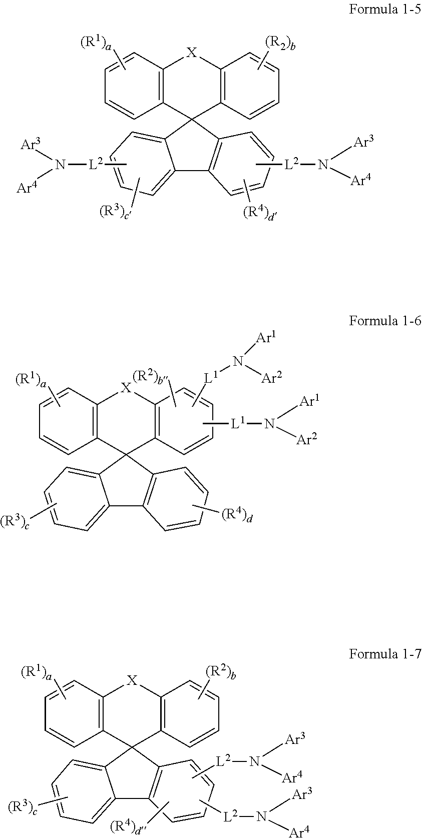
wherein:
1) X, R¹, R², R³, R⁴, a, b, c, d, L¹, L², Ar¹, Ar², Ar³ and Ar⁴ are the same as defined in claim 1,
2) a′, b′, c′ and d′ are each independently an integer of 0 to 3, and
3) b″ and d″ are each independently an integer of 0 to 2.

3. The organic electronic element of claim 1, wherein the compound represented by Formula 1 is represented by any one of Formulas 1-8 to 1-10: 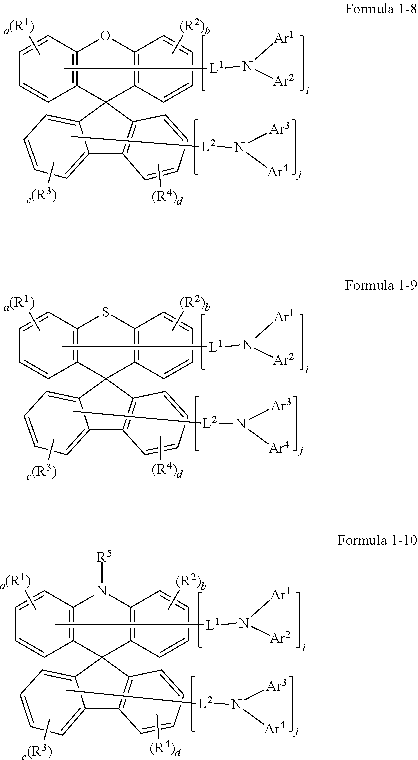
wherein R¹, R², R³, R⁴, a, b, c, d, L¹, L², Ar¹, Ar², Ar³, Ar⁴, i and j are the same as defined in claim 1.

4. The organic electronic element of claim 1, wherein at least one of Ar¹ to Ar⁴ in Formula 1 is represented by Formula B-1: 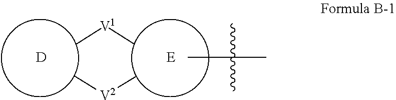
wherein:
1) V¹ and V² are each independently a single bond, NR¹⁰, CR¹¹R¹², O or S,
2) R¹⁰, R¹¹ and R¹² are the same as the definition of R⁵ in claim 1, or R¹¹ and R¹² may be bonded to each other to form a ring,
3) Ring D and Ring E are each independently a C₆-C₂₀ aryl group; or C₄-C₂₀ heterocyclic group.

5. The organic electronic element of claim 1, wherein in any one of R¹ to R⁴ in Formula 1, an adjacent pair is bonded to each other to form any one of benzene, indole, indene, benzofuran, and benzothiophene.

6. The organic electronic element of claim 1, wherein the compound represented by Formula 1 is any one of the following compounds:

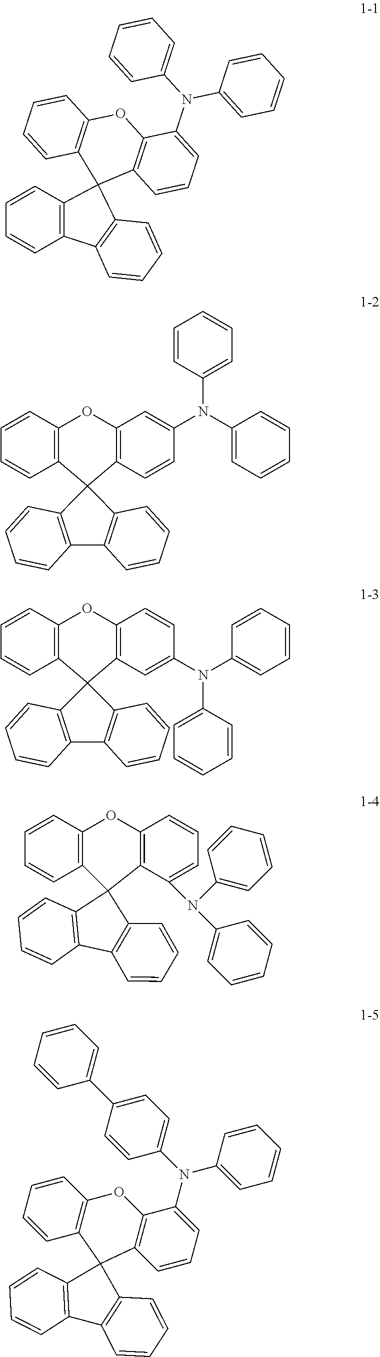

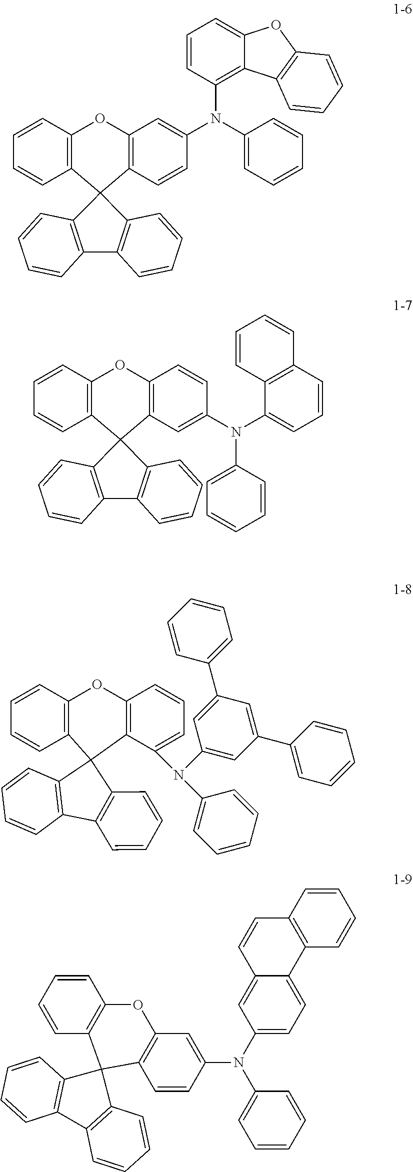 [SMILES: c1ccc(N(c2ccc3c(c2)Oc2ccccc2C32c3ccccc3-c3ccccc32)c2cccc3oc4ccccc4c23)cc1 ; c1ccc(N(c2ccc3c(c2)C2(c4ccccc4O3)c3ccccc3-c3ccccc32)c2cccc3ccccc23)cc1 ; c1ccc(-c2cc(-c3ccccc3)cc(N(c3ccccc3)c3cccc4c3C3(c5ccccc5O4)c4ccccc4-c4ccccc43)c2)cc1 ; c1ccc(N(c2ccc3c(c2)Oc2ccccc2C32c3ccccc3-c3ccccc32)c2ccc3c(ccc4ccccc43)c2)cc1]

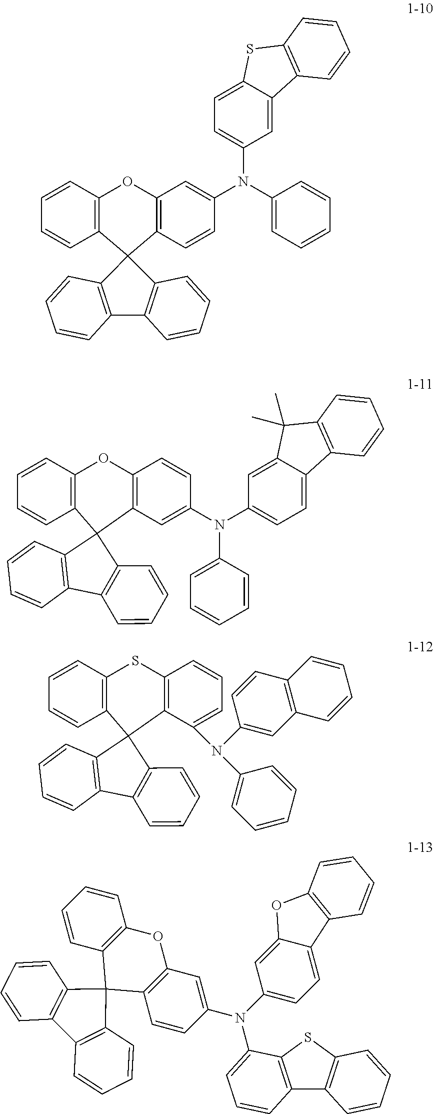

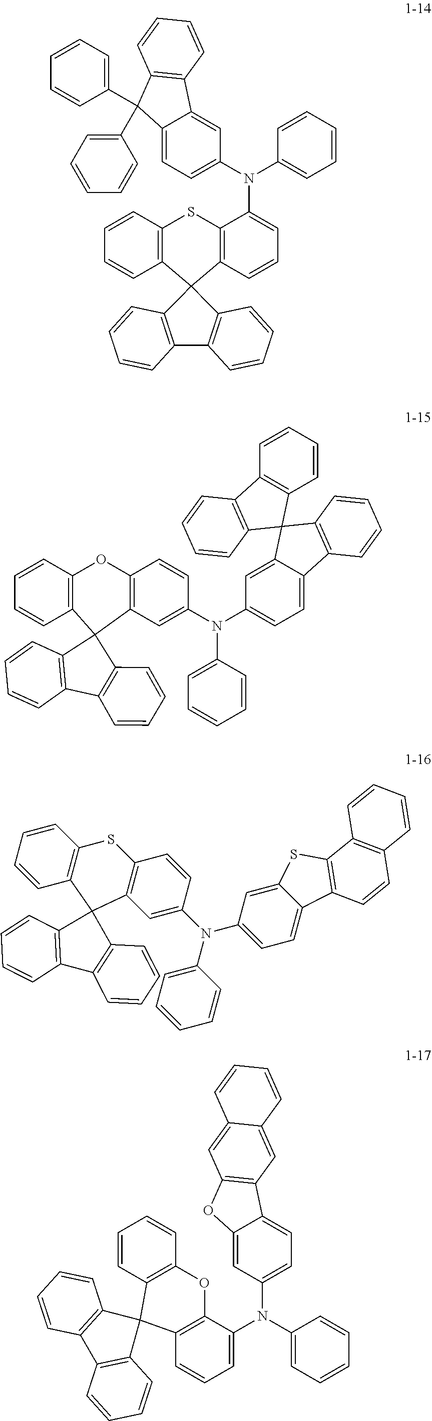 [SMILES: c1ccc(N(c2ccc3c(c2)-c2ccccc2C3(c2ccccc2)c2ccccc2)c2cccc3c2Sc2ccccc2C32c3ccccc3-c3ccccc32)cc1 ; c1ccc(N(c2ccc3c(c2)C2(c4ccccc4O3)c3ccccc3-c3ccccc32)c2ccc3c(c2)C2(c4ccccc4-c4ccccc42)c2ccccc2-3)cc1 ; c1ccc(N(c2ccc3c(c2)C2(c4ccccc4S3)c3ccccc3-c3ccccc32)c2ccc3c(c2)sc2c4ccccc4ccc32)cc1 ; c1ccc(N(c2ccc3c(c2)oc2cc4ccccc4cc23)c2cccc3c2Oc2ccccc2C32c3ccccc3-c3ccccc32)cc1]

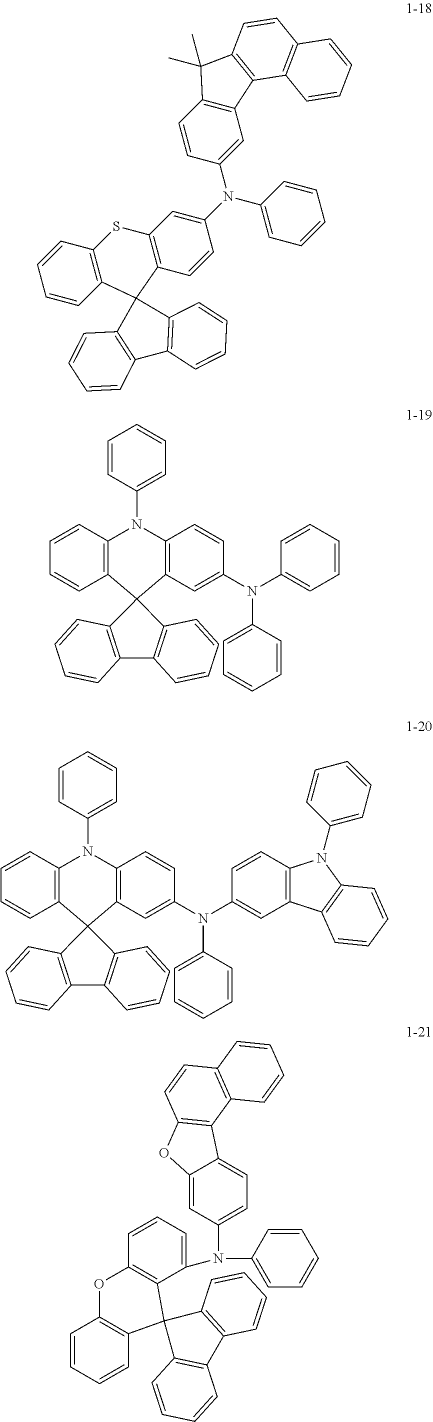

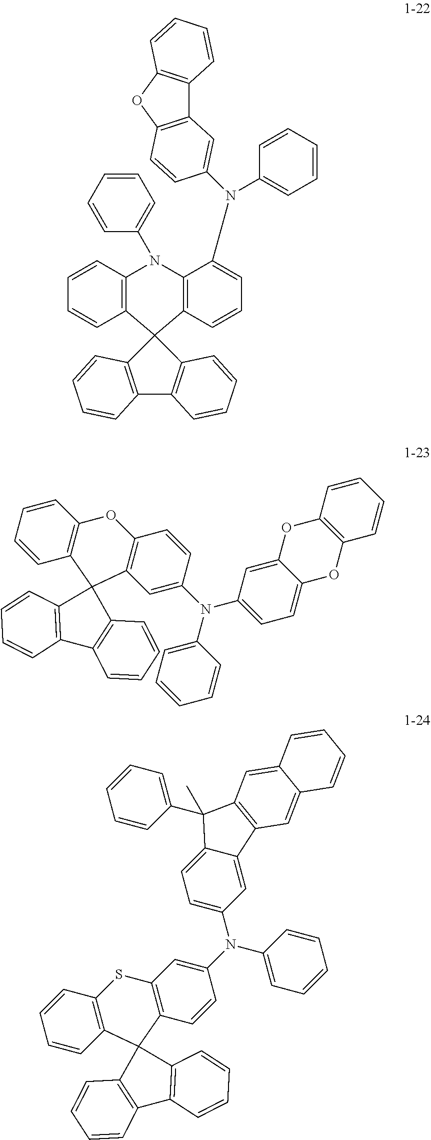 [SMILES: c1ccc(N(c2ccc3oc4ccccc4c3c2)c2cccc3c2N(c2ccccc2)c2ccccc2C32c3ccccc3-c3ccccc32)cc1 ; c1ccc(N(c2ccc3c(c2)Oc2ccccc2O3)c2ccc3c(c2)C2(c4ccccc4O3)c3ccccc3-c3ccccc32)cc1 ; CC1(c2ccccc2)c2ccc(N(c3ccccc3)c3ccc4c(c3)Sc3ccccc3C43c4ccccc4-c4ccccc43)cc2-c2cc3ccccc3cc21]

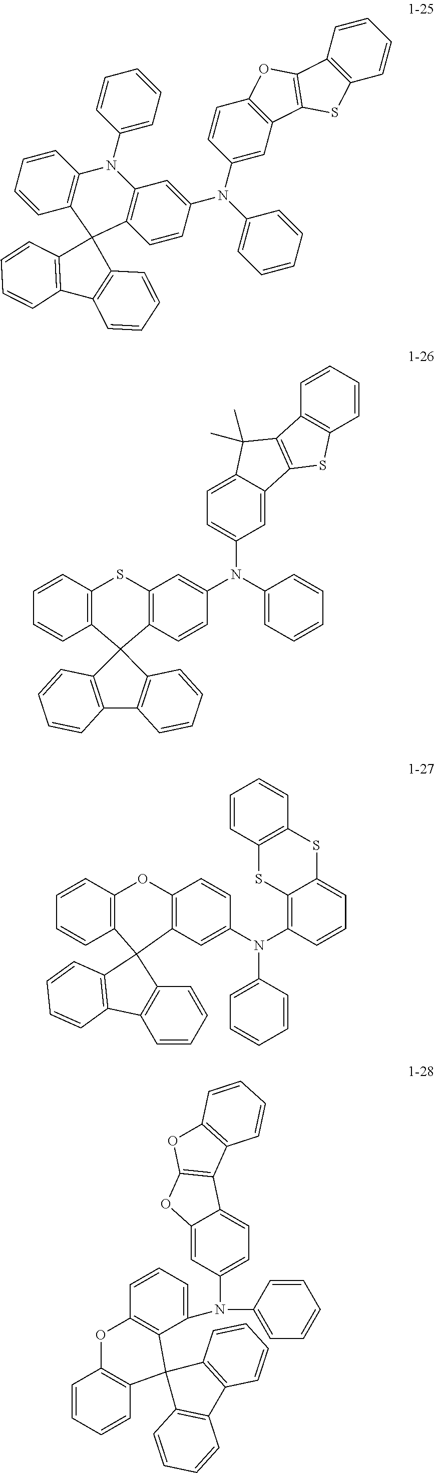

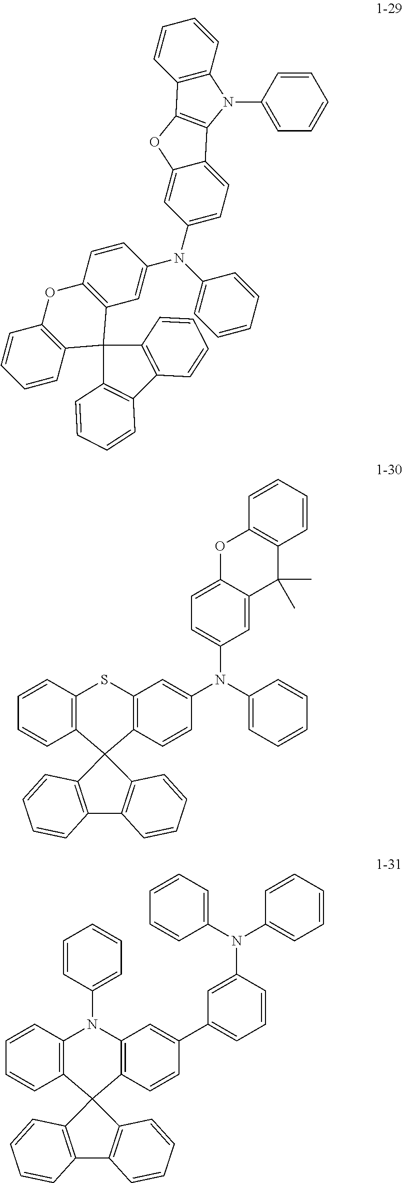

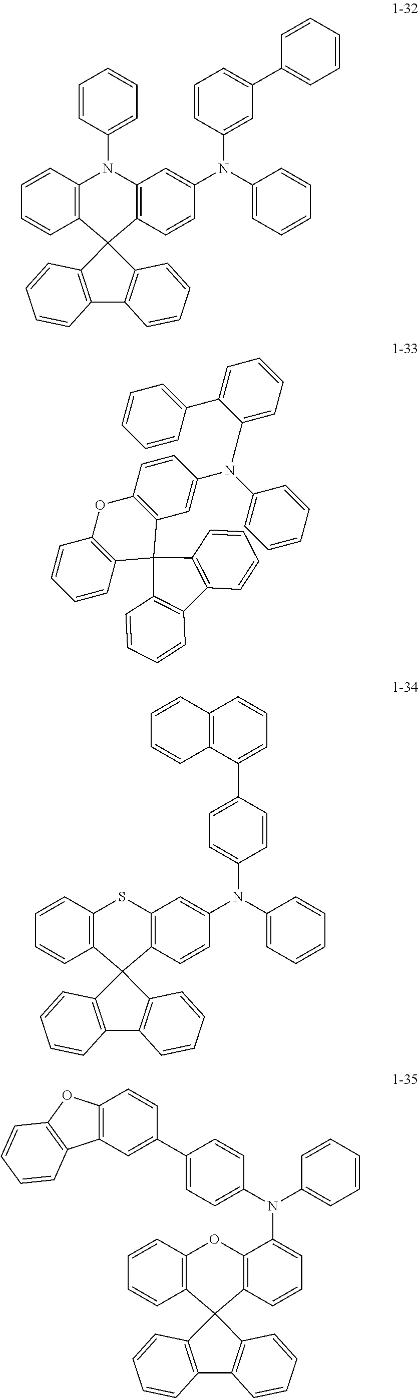

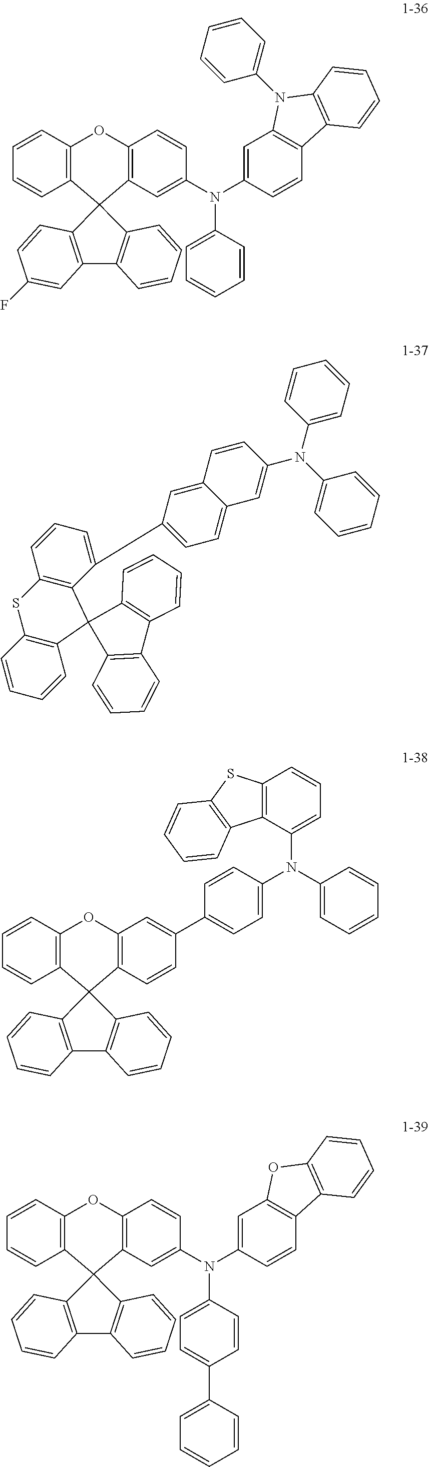 [SMILES: Fc1ccc2c(c1)-c1ccccc1C21c2ccccc2Oc2ccc(N(c3ccccc3)c3ccc4c5ccccc5n(-c5ccccc5)c4c3)cc21 ; c1ccc(N(c2ccccc2)c2ccc3cc(-c4cccc5c4C4(c6ccccc6S5)c5ccccc5-c5ccccc54)ccc3c2)cc1 ; c1ccc(N(c2ccc(-c3ccc4c(c3)Oc3ccccc3C43c4ccccc4-c4ccccc43)cc2)c2cccc3sc4ccccc4c23)cc1 ; c1ccc(-c2ccc(N(c3ccc4c(c3)C3(c5ccccc5O4)c4ccccc4-c4ccccc43)c3ccc4c(c3)oc3ccccc34)cc2)cc1]

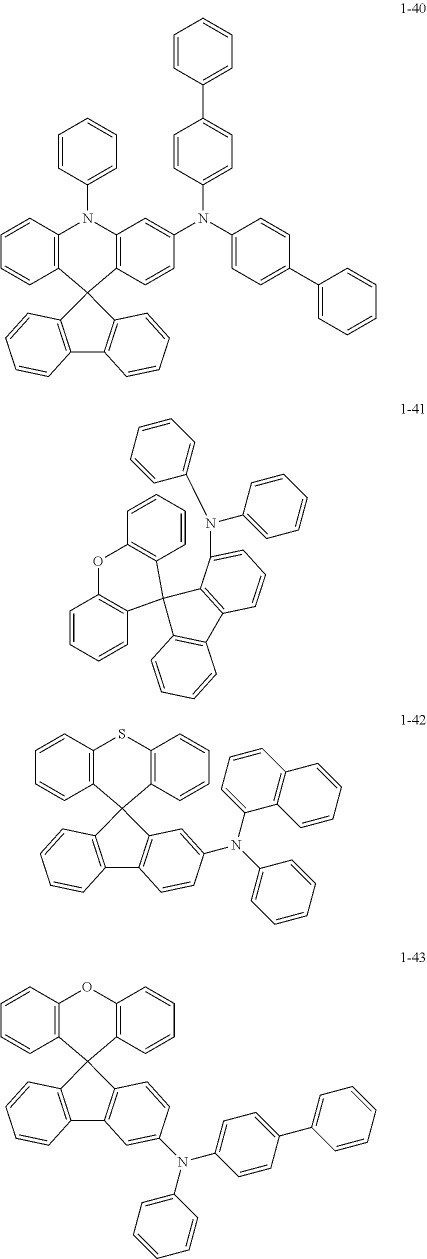

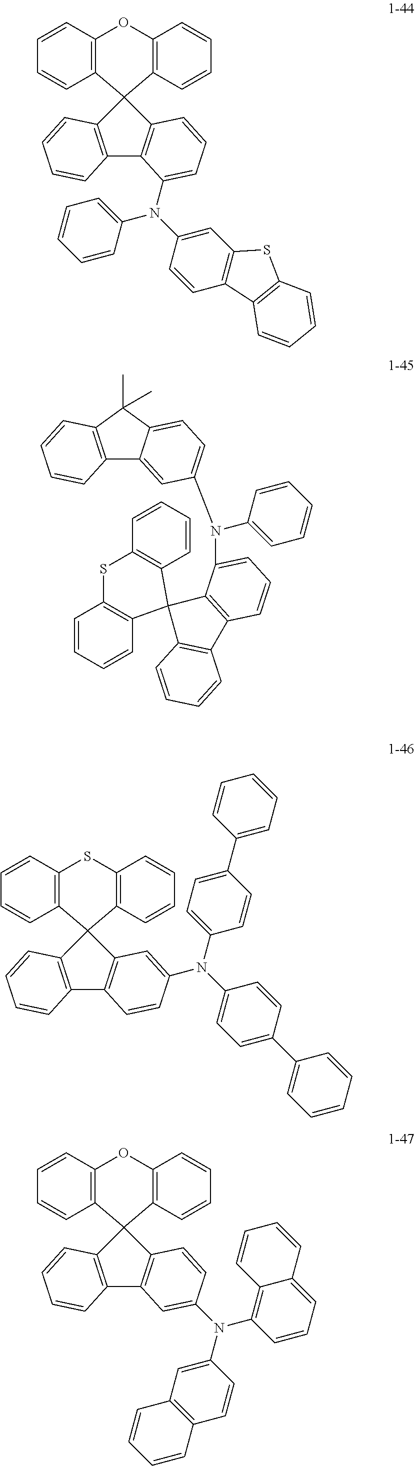 [SMILES: c1ccc(N(c2ccc3c(c2)sc2ccccc23)c2cccc3c2-c2ccccc2C32c3ccccc3Oc3ccccc32)cc1 ; CC1(C)c2ccccc2-c2cc(N(c3ccccc3)c3cccc4c3C3(c5ccccc5Sc5ccccc53)c3ccccc3-4)ccc21 ; c1ccc(-c2ccc(N(c3ccc(-c4ccccc4)cc3)c3ccc4c(c3)C3(c5ccccc5Sc5ccccc53)c3ccccc3-4)cc2)cc1 ; c1ccc2c(c1)Oc1ccccc1C21c2ccccc2-c2cc(N(c3ccc4ccccc4c3)c3cccc4ccccc34)ccc21]

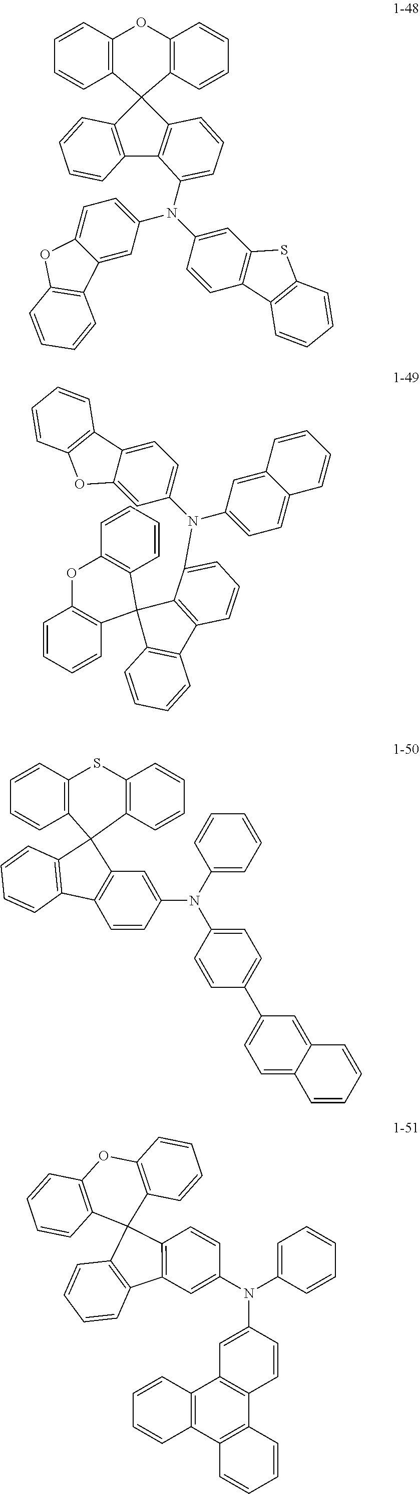

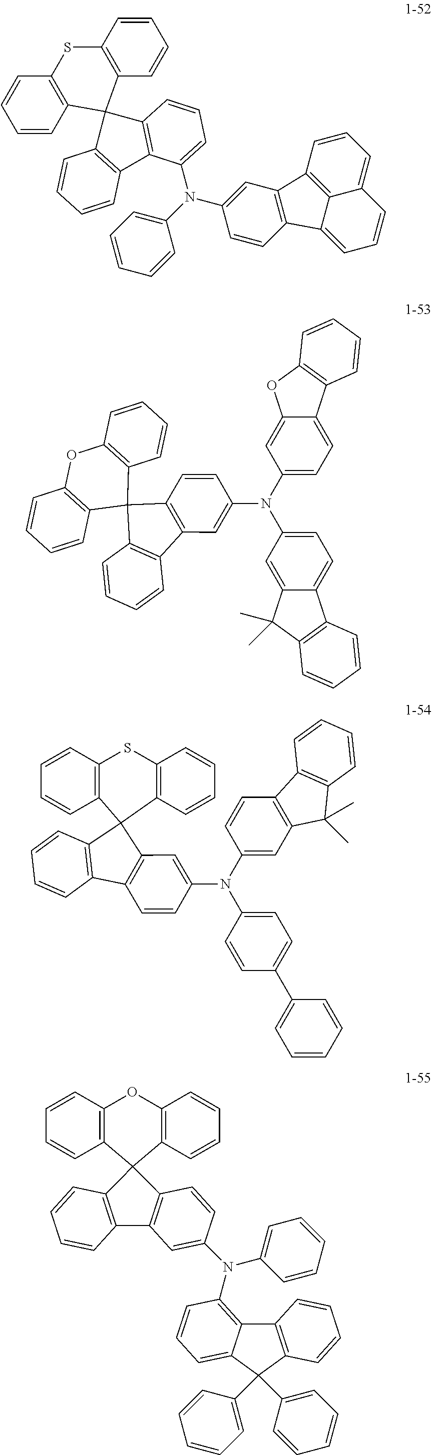

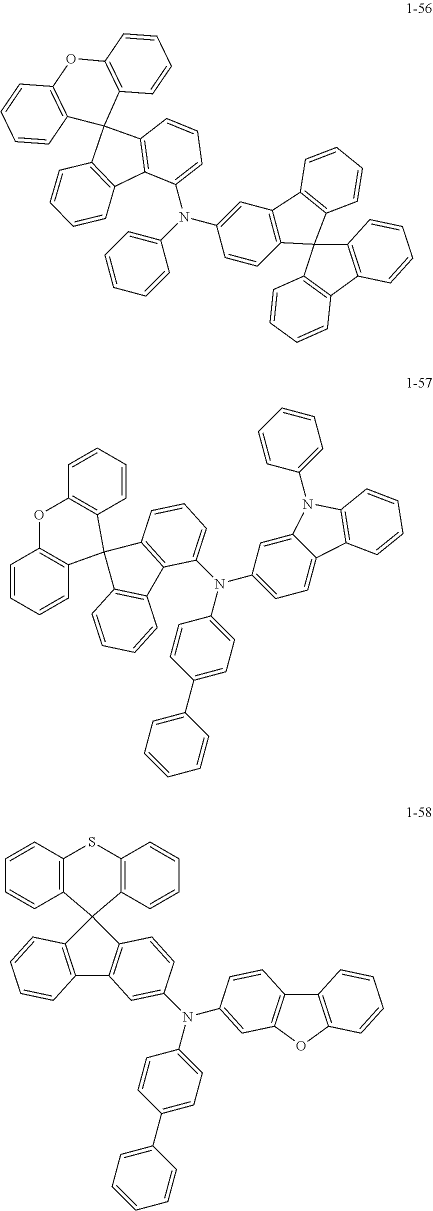 [SMILES: c1ccc(N(c2ccc3c(c2)-c2ccccc2C32c3ccccc3-c3ccccc32)c2cccc3c2-c2ccccc2C32c3ccccc3Oc3ccccc32)cc1 ; c1ccc(-c2ccc(N(c3ccc4c5ccccc5n(-c5ccccc5)c4c3)c3cccc4c3-c3ccccc3C43c4ccccc4Oc4ccccc43)cc2)cc1 ; c1ccc(-c2ccc(N(c3ccc4c(c3)-c3ccccc3C43c4ccccc4Sc4ccccc43)c3ccc4c(c3)oc3ccccc34)cc2)cc1]

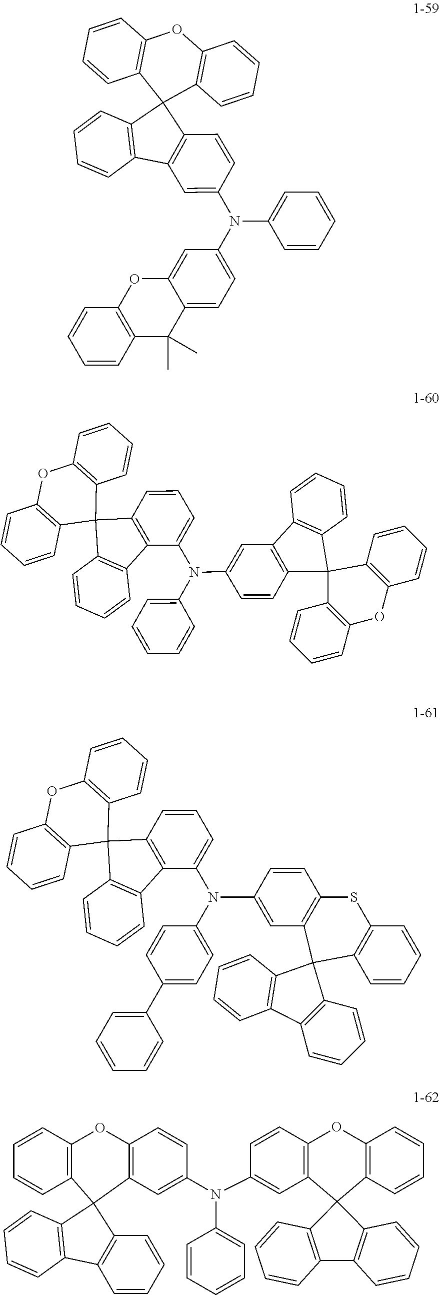 [SMILES: CC1(C)c2ccccc2Oc2cc(N(c3ccccc3)c3ccc4c(c3)-c3ccccc3C43c4ccccc4Oc4ccccc43)ccc21 ; c1ccc(N(c2ccc3c(c2)-c2ccccc2C32c3ccccc3Oc3ccccc32)c2cccc3c2-c2ccccc2C32c3ccccc3Oc3ccccc32)cc1 ; c1ccc(-c2ccc(N(c3ccc4c(c3)C3(c5ccccc5S4)c4ccccc4-c4ccccc43)c3cccc4c3-c3ccccc3C43c4ccccc4Oc4ccccc43)cc2)cc1 ; c1ccc(N(c2ccc3c(c2)C2(c4ccccc4O3)c3ccccc3-c3ccccc32)c2ccc3c(c2)C2(c4ccccc4O3)c3ccccc3-c3ccccc32)cc1]

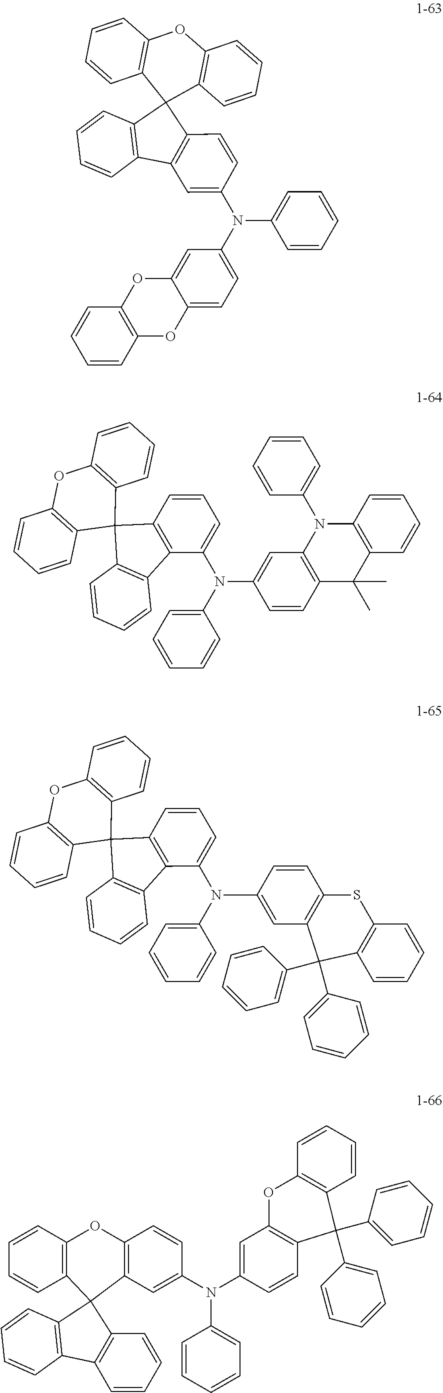 [SMILES: c1ccc(N(c2ccc3c(c2)Oc2ccccc2O3)c2ccc3c(c2)-c2ccccc2C32c3ccccc3Oc3ccccc32)cc1 ; CC1(C)c2ccccc2N(c2ccccc2)c2cc(N(c3ccccc3)c3cccc4c3-c3ccccc3C43c4ccccc4Oc4ccccc43)ccc21 ; c1ccc(N(c2ccc3c(c2)C(c2ccccc2)(c2ccccc2)c2ccccc2S3)c2cccc3c2-c2ccccc2C32c3ccccc3Oc3ccccc32)cc1 ; c1ccc(N(c2ccc3c(c2)Oc2ccccc2C3(c2ccccc2)c2ccccc2)c2ccc3c(c2)C2(c4ccccc4O3)c3ccccc3-c3ccccc32)cc1]

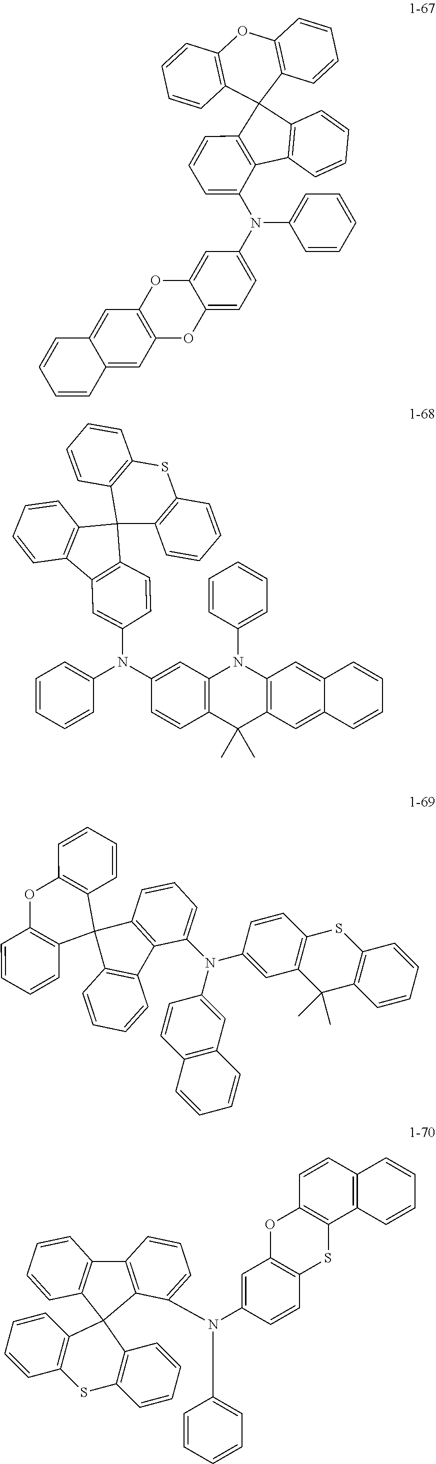

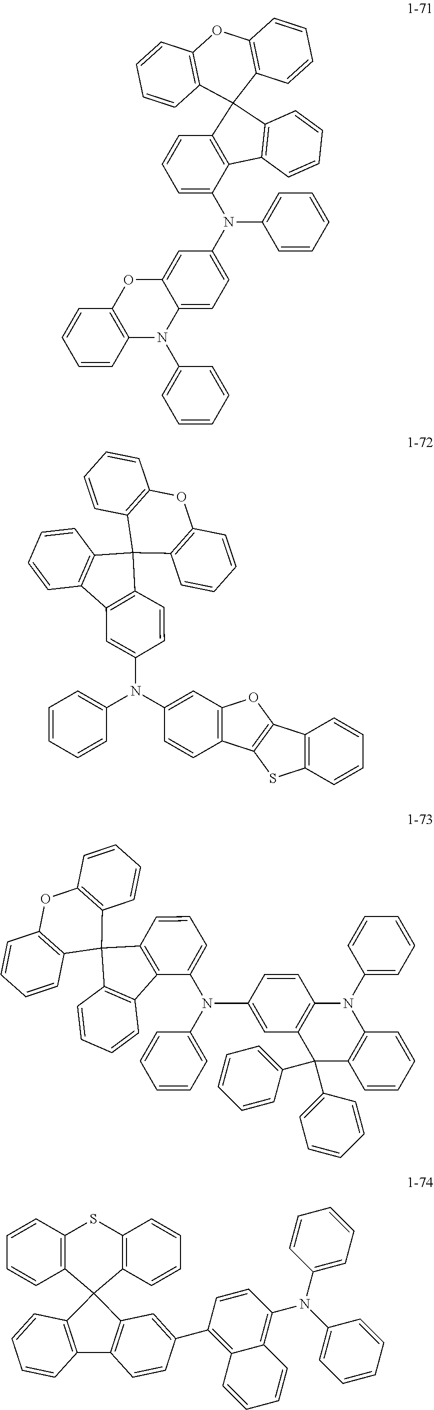 [SMILES: c1ccc(N(c2ccc3c(c2)Oc2ccccc2N3c2ccccc2)c2cccc3c2-c2ccccc2C32c3ccccc3Oc3ccccc32)cc1 ; c1ccc(N(c2ccc3c(c2)-c2ccccc2C32c3ccccc3Oc3ccccc32)c2ccc3c(c2)oc2c4ccccc4sc32)cc1 ; c1ccc(N(c2ccc3c(c2)C(c2ccccc2)(c2ccccc2)c2ccccc2N3c2ccccc2)c2cccc3c2-c2ccccc2C32c3ccccc3Oc3ccccc32)cc1 ; c1ccc(N(c2ccccc2)c2ccc(-c3ccc4c(c3)C3(c5ccccc5Sc5ccccc53)c3ccccc3-4)c3ccccc23)cc1]

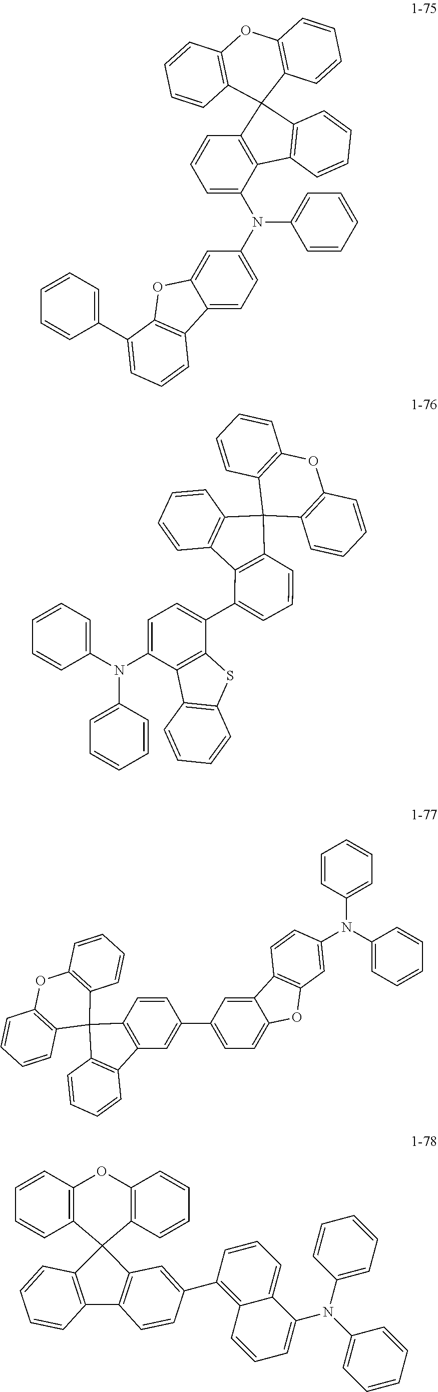 [SMILES: c1ccc(-c2cccc3c2oc2cc(N(c4ccccc4)c4cccc5c4-c4ccccc4C54c5ccccc5Oc5ccccc54)ccc23)cc1 ; c1ccc(N(c2ccccc2)c2ccc(-c3cccc4c3-c3ccccc3C43c4ccccc4Oc4ccccc43)c3sc4ccccc4c23)cc1 ; c1ccc(N(c2ccccc2)c2ccc3c(c2)oc2ccc(-c4ccc5c(c4)-c4ccccc4C54c5ccccc5Oc5ccccc54)cc23)cc1 ; c1ccc(N(c2ccccc2)c2cccc3c(-c4ccc5c(c4)C4(c6ccccc6Oc6ccccc64)c4ccccc4-5)cccc23)cc1]

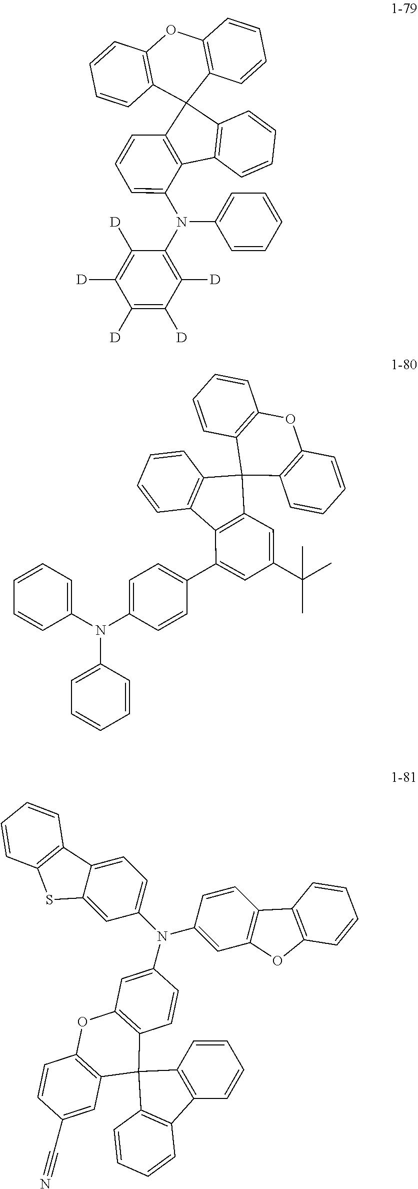 [SMILES: [2H]c1c([2H])c([2H])c(N(c2ccccc2)c2cccc3c2-c2ccccc2C32c3ccccc3Oc3ccccc32)c([2H])c1[2H] ; CC(C)(C)c1cc(-c2ccc(N(c3ccccc3)c3ccccc3)cc2)c2c(c1)C1(c3ccccc3Oc3ccccc31)c1ccccc1-2 ; N#Cc1ccc2c(c1)C1(c3ccc(N(c4ccc5c(c4)oc4ccccc45)c4ccc5c(c4)sc4ccccc45)cc3O2)c2ccccc2-c2ccccc21]

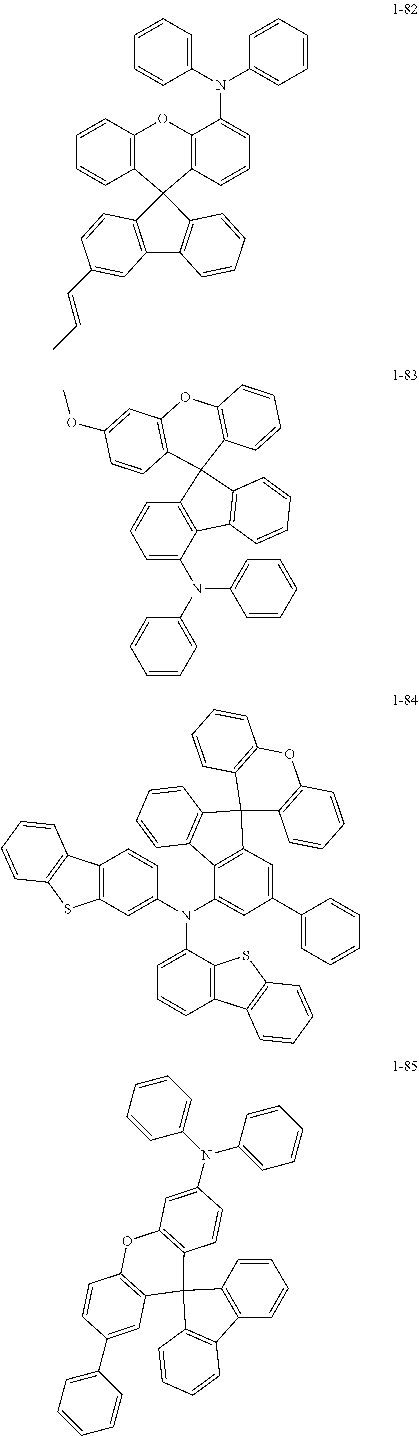

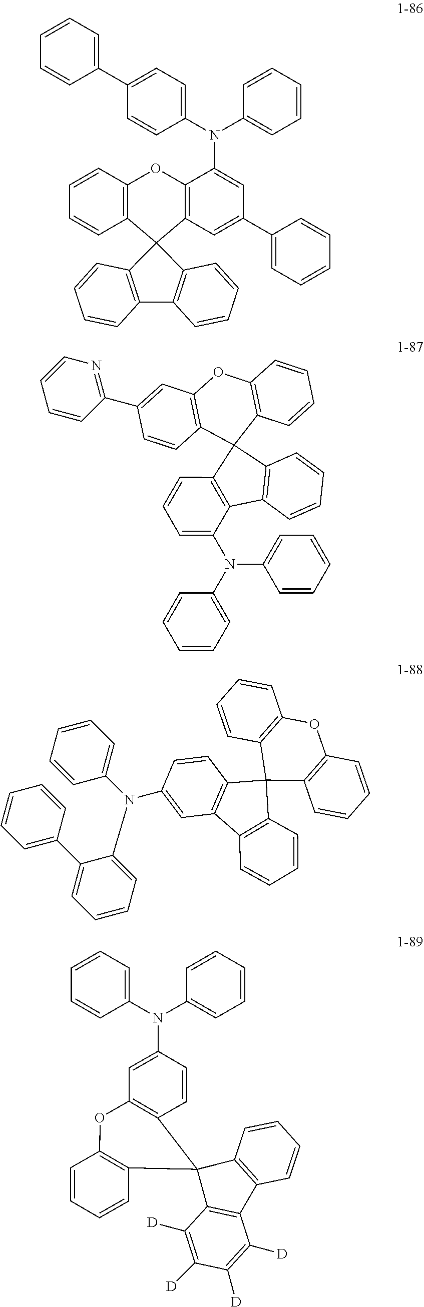

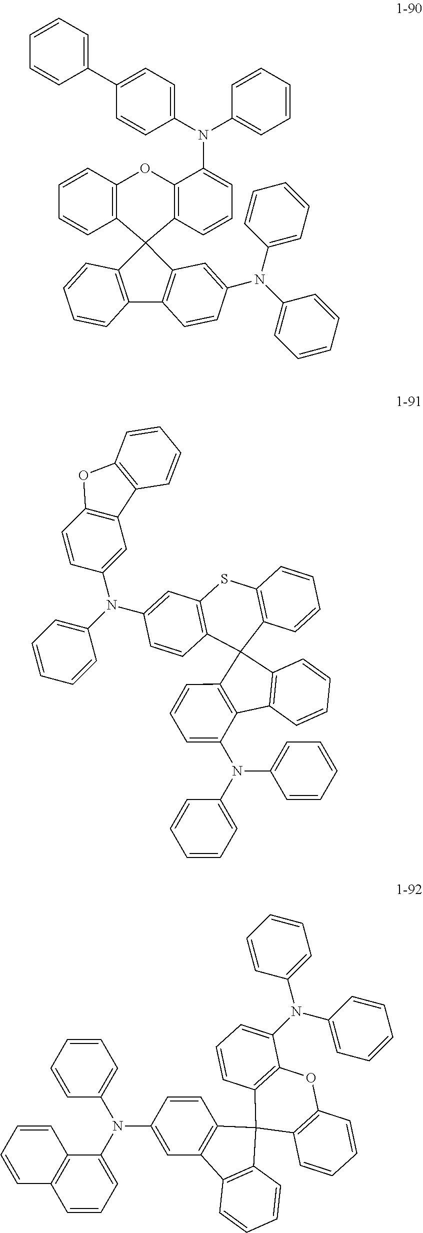 [SMILES: c1ccc(-c2ccc(N(c3ccccc3)c3cccc4c3Oc3ccccc3C43c4ccccc4-c4ccc(N(c5ccccc5)c5ccccc5)cc43)cc2)cc1 ; c1ccc(N(c2ccc3c(c2)Sc2ccccc2C32c3ccccc3-c3c(N(c4ccccc4)c4ccccc4)cccc32)c2ccc3oc4ccccc4c3c2)cc1 ; c1ccc(N(c2ccccc2)c2cccc3c2Oc2ccccc2C32c3ccccc3-c3cc(N(c4ccccc4)c4cccc5ccccc45)ccc32)cc1]

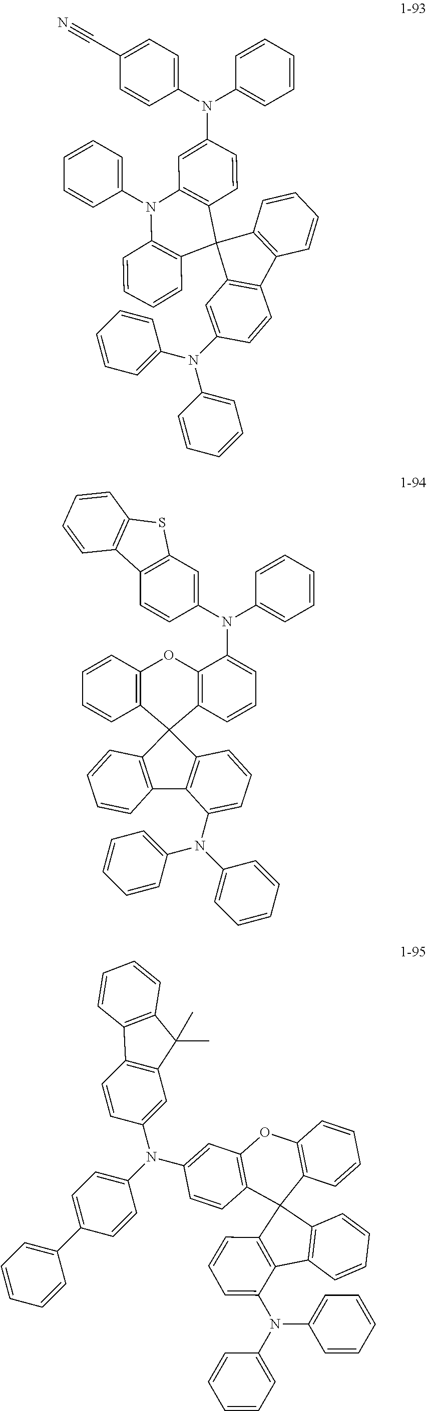 [SMILES: N#Cc1ccc(N(c2ccccc2)c2ccc3c(c2)N(c2ccccc2)c2ccccc2C32c3ccccc3-c3ccc(N(c4ccccc4)c4ccccc4)cc32)cc1 ; c1ccc(N(c2ccc3c(c2)sc2ccccc23)c2cccc3c2Oc2ccccc2C32c3ccccc3-c3c(N(c4ccccc4)c4ccccc4)cccc32)cc1 ; CC1(C)c2ccccc2-c2ccc(N(c3ccc(-c4ccccc4)cc3)c3ccc4c(c3)Oc3ccccc3C43c4ccccc4-c4c(N(c5ccccc5)c5ccccc5)cccc43)cc21]

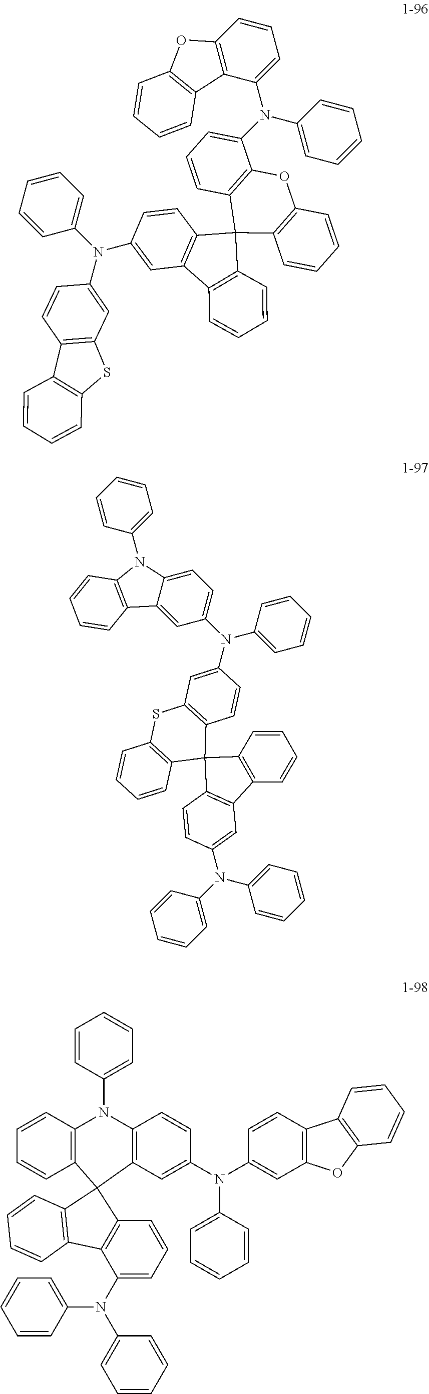 [SMILES: c1ccc(N(c2ccc3c(c2)-c2ccccc2C32c3ccccc3Oc3c(N(c4ccccc4)c4cccc5oc6ccccc6c45)cccc32)c2ccc3c(c2)sc2ccccc23)cc1 ; c1ccc(N(c2ccccc2)c2ccc3c(c2)-c2ccccc2C32c3ccccc3Sc3cc(N(c4ccccc4)c4ccc5c(c4)c4ccccc4n5-c4ccccc4)ccc32)cc1 ; c1ccc(N(c2ccc3c(c2)C2(c4ccccc4-c4c(N(c5ccccc5)c5ccccc5)cccc42)c2ccccc2N3c2ccccc2)c2ccc3c(c2)oc2ccccc23)cc1]

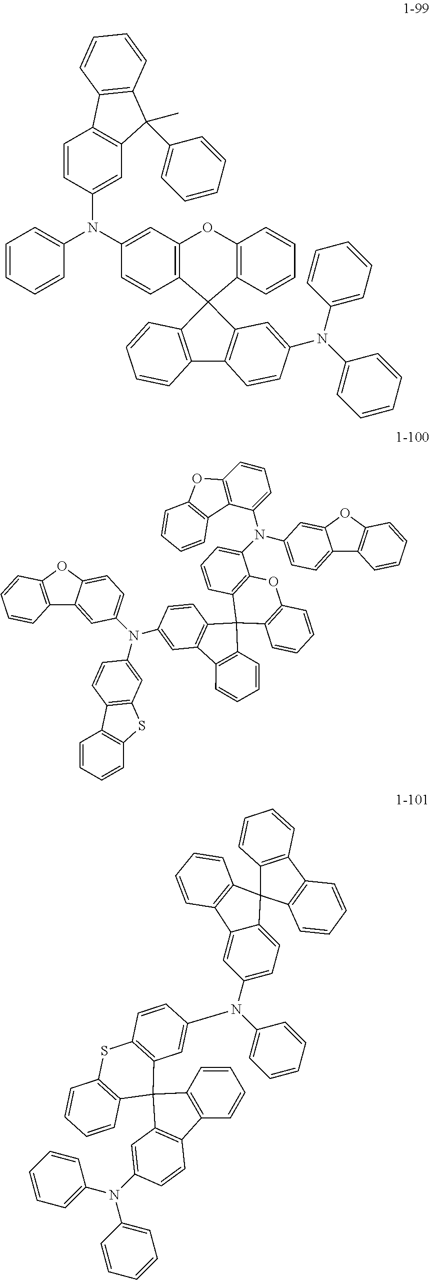 [SMILES: CC1(c2ccccc2)c2ccccc2-c2ccc(N(c3ccccc3)c3ccc4c(c3)Oc3ccccc3C43c4ccccc4-c4ccc(N(c5ccccc5)c5ccccc5)cc43)cc21 ; c1ccc2c(c1)Oc1c(N(c3ccc4c(c3)oc3ccccc34)c3cccc4oc5ccccc5c34)cccc1C21c2ccccc2-c2cc(N(c3ccc4c(c3)sc3ccccc34)c3ccc4oc5ccccc5c4c3)ccc21 ; c1ccc(N(c2ccc3c(c2)-c2ccccc2C32c3ccccc3-c3ccccc32)c2ccc3c(c2)C2(c4ccccc4S3)c3ccccc3-c3ccc(N(c4ccccc4)c4ccccc4)cc32)cc1]

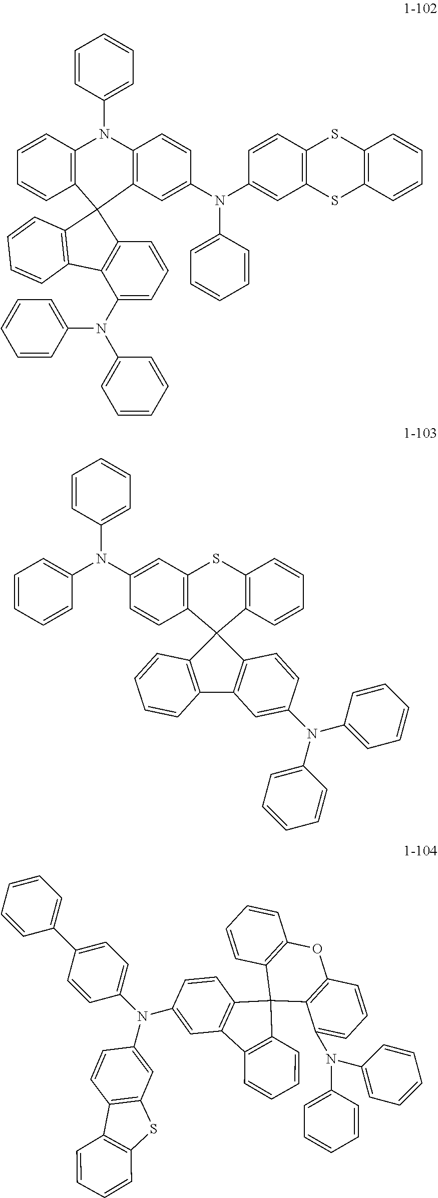

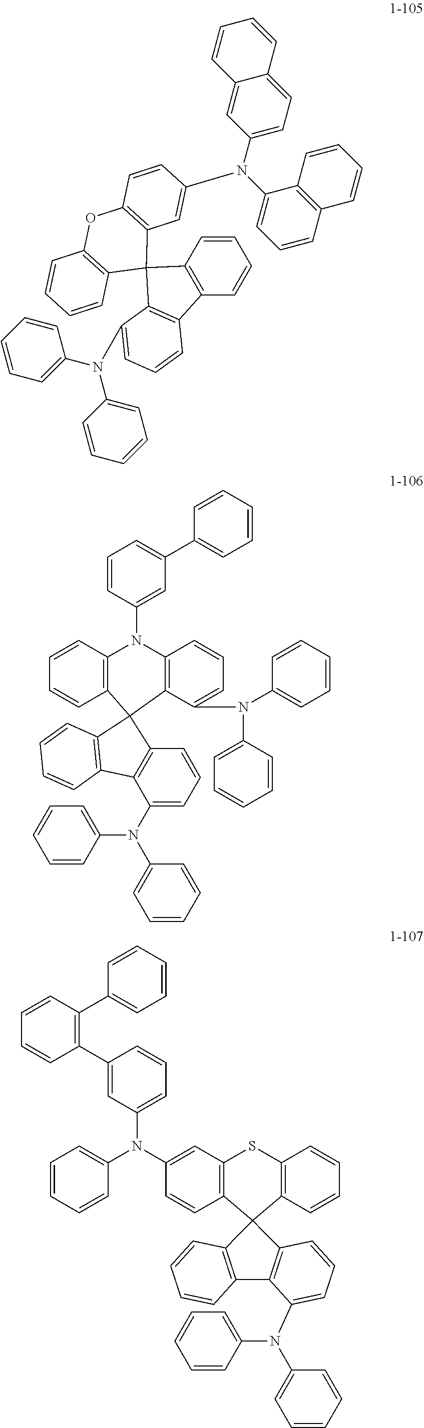

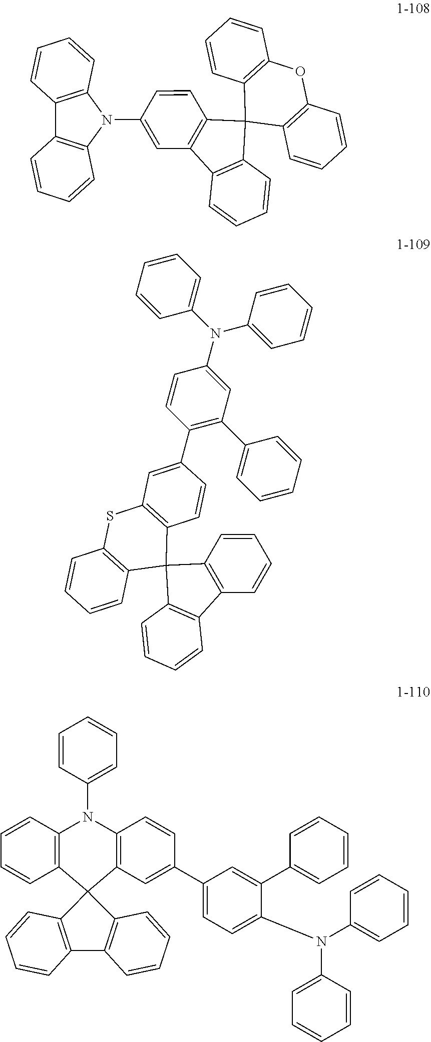 [SMILES: c1ccc2c(c1)Oc1ccccc1C21c2ccccc2-c2cc(-n3c4ccccc4c4ccccc43)ccc21 ; c1ccc(-c2cc(N(c3ccccc3)c3ccccc3)ccc2-c2ccc3c(c2)Sc2ccccc2C32c3ccccc3-c3ccccc32)cc1 ; c1ccc(-c2cc(-c3ccc4c(c3)C3(c5ccccc5-c5ccccc53)c3ccccc3N4c3ccccc3)ccc2N(c2ccccc2)c2ccccc2)cc1]

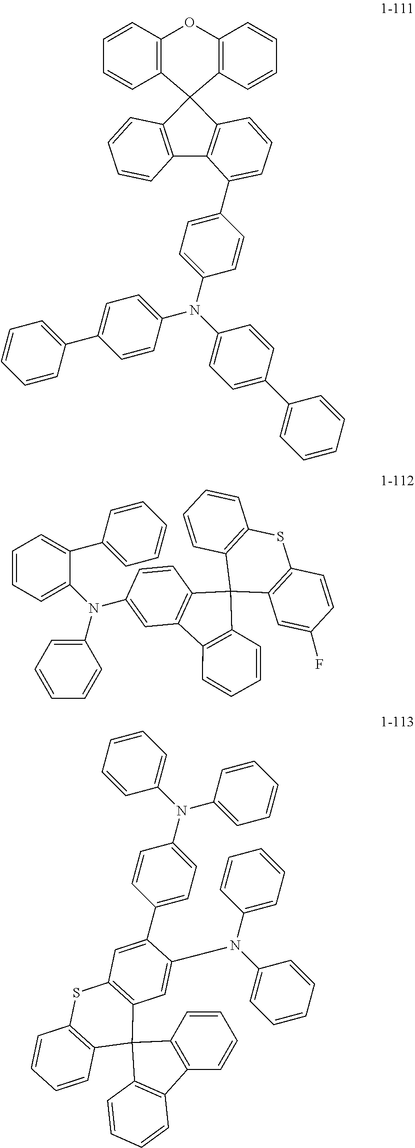 [SMILES: c1ccc(-c2ccc(N(c3ccc(-c4ccccc4)cc3)c3ccc(-c4cccc5c4-c4ccccc4C54c5ccccc5Oc5ccccc54)cc3)cc2)cc1 ; Fc1ccc2c(c1)C1(c3ccccc3S2)c2ccccc2-c2cc(N(c3ccccc3)c3ccccc3-c3ccccc3)ccc21 ; c1ccc(N(c2ccccc2)c2ccc(-c3cc4c(cc3N(c3ccccc3)c3ccccc3)C3(c5ccccc5S4)c4ccccc4-c4ccccc43)cc2)cc1]

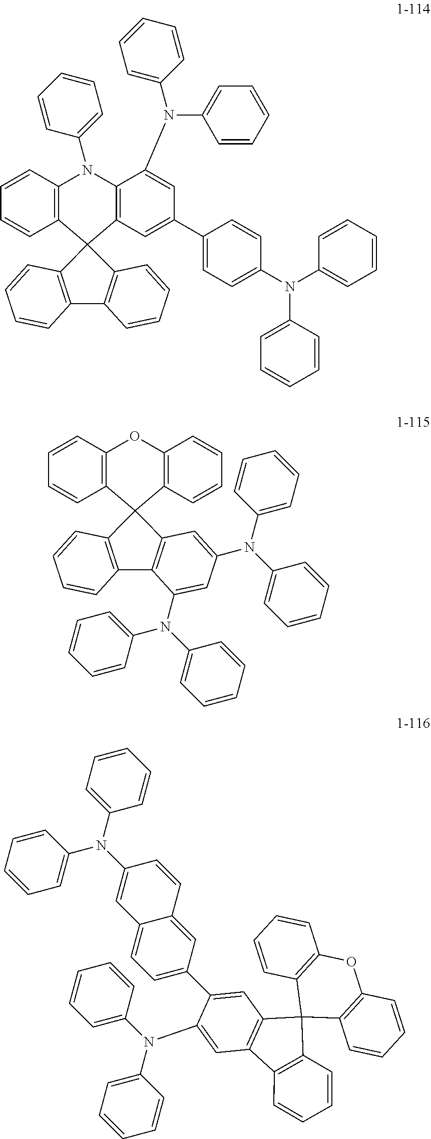 [SMILES: c1ccc(N(c2ccccc2)c2ccc(-c3cc(N(c4ccccc4)c4ccccc4)c4c(c3)C3(c5ccccc5-c5ccccc53)c3ccccc3N4c3ccccc3)cc2)cc1 ; c1ccc(N(c2ccccc2)c2cc(N(c3ccccc3)c3ccccc3)c3c(c2)C2(c4ccccc4Oc4ccccc42)c2ccccc2-3)cc1 ; c1ccc(N(c2ccccc2)c2ccc3cc(-c4cc5c(cc4N(c4ccccc4)c4ccccc4)-c4ccccc4C54c5ccccc5Oc5ccccc54)ccc3c2)cc1]

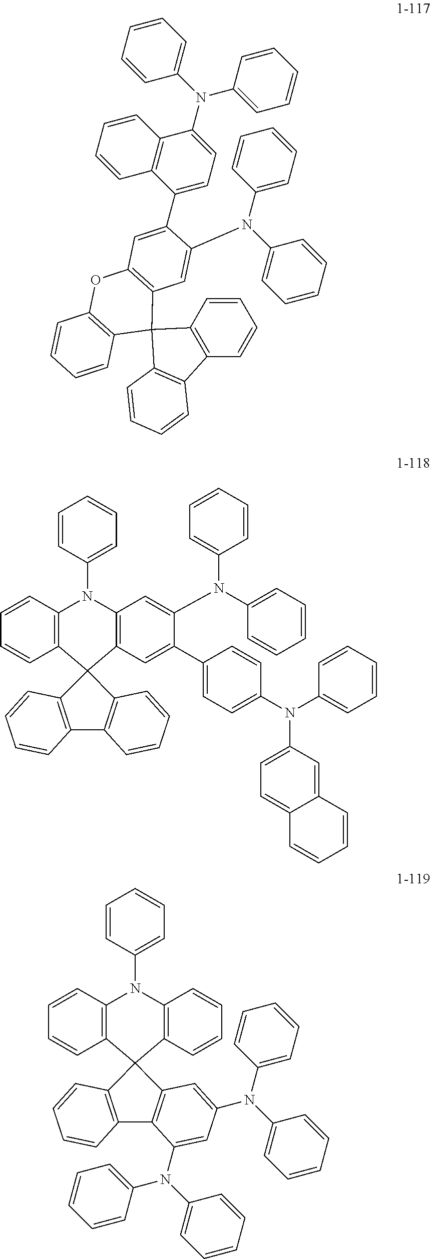 [SMILES: c1ccc(N(c2ccccc2)c2cc3c(cc2-c2ccc(N(c4ccccc4)c4ccccc4)c4ccccc24)Oc2ccccc2C32c3ccccc3-c3ccccc32)cc1 ; c1ccc(N(c2ccc(-c3cc4c(cc3N(c3ccccc3)c3ccccc3)N(c3ccccc3)c3ccccc3C43c4ccccc4-c4ccccc43)cc2)c2ccc3ccccc3c2)cc1 ; c1ccc(N(c2ccccc2)c2cc(N(c3ccccc3)c3ccccc3)c3c(c2)C2(c4ccccc4-3)c3ccccc3N(c3ccccc3)c3ccccc32)cc1]

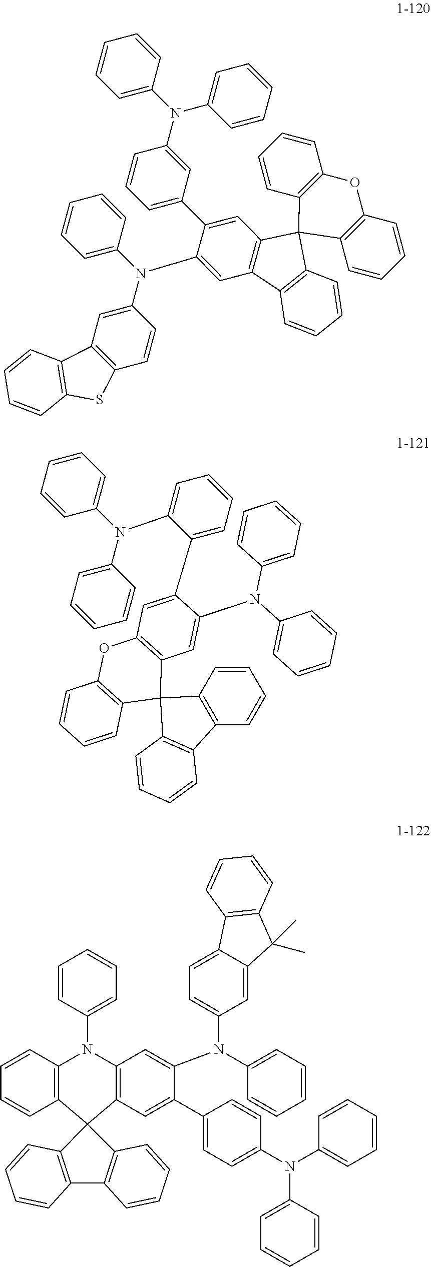

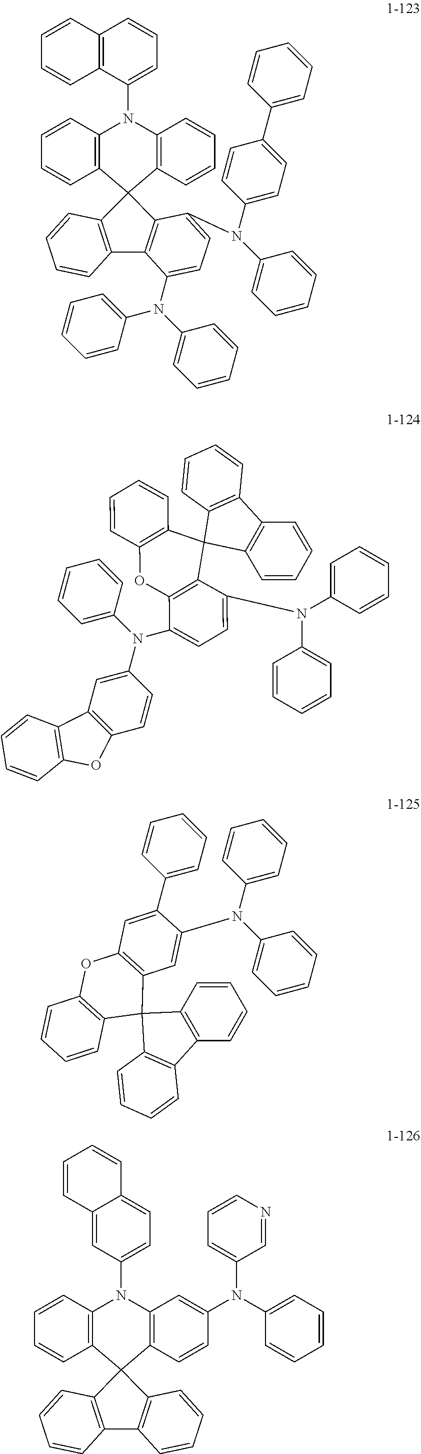 [SMILES: c1ccc(-c2ccc(N(c3ccccc3)c3ccc(N(c4ccccc4)c4ccccc4)c4c3C3(c5ccccc5-4)c4ccccc4N(c4cccc5ccccc45)c4ccccc43)cc2)cc1 ; c1ccc(N(c2ccc3oc4ccccc4c3c2)c2ccc(N(c3ccccc3)c3ccccc3)c3c2Oc2ccccc2C32c3ccccc3-c3ccccc32)cc1 ; c1ccc(-c2cc3c(cc2N(c2ccccc2)c2ccccc2)C2(c4ccccc4O3)c3ccccc3-c3ccccc32)cc1 ; c1ccc(N(c2cccnc2)c2ccc3c(c2)N(c2ccc4ccccc4c2)c2ccccc2C32c3ccccc3-c3ccccc32)cc1]

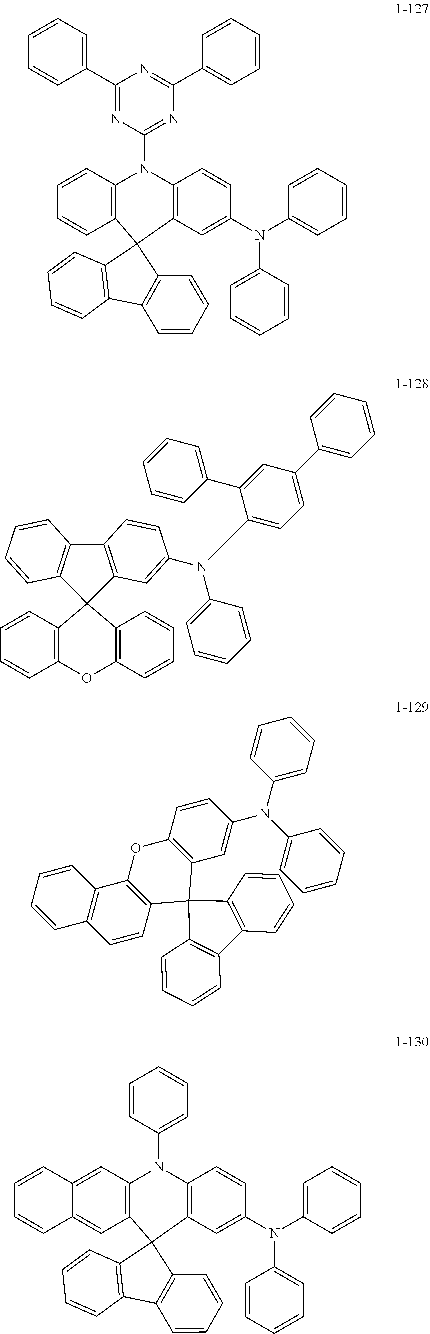 [SMILES: c1ccc(-c2nc(-c3ccccc3)nc(N3c4ccccc4C4(c5ccccc5-c5ccccc54)c4cc(N(c5ccccc5)c5ccccc5)ccc43)n2)cc1 ; c1ccc(-c2ccc(N(c3ccccc3)c3ccc4c(c3)C3(c5ccccc5Oc5ccccc53)c3ccccc3-4)c(-c3ccccc3)c2)cc1 ; c1ccc(N(c2ccccc2)c2ccc3c(c2)C2(c4ccccc4-c4ccccc42)c2ccc4ccccc4c2O3)cc1 ; c1ccc(N(c2ccccc2)c2ccc3c(c2)C2(c4ccccc4-c4ccccc42)c2cc4ccccc4cc2N3c2ccccc2)cc1]

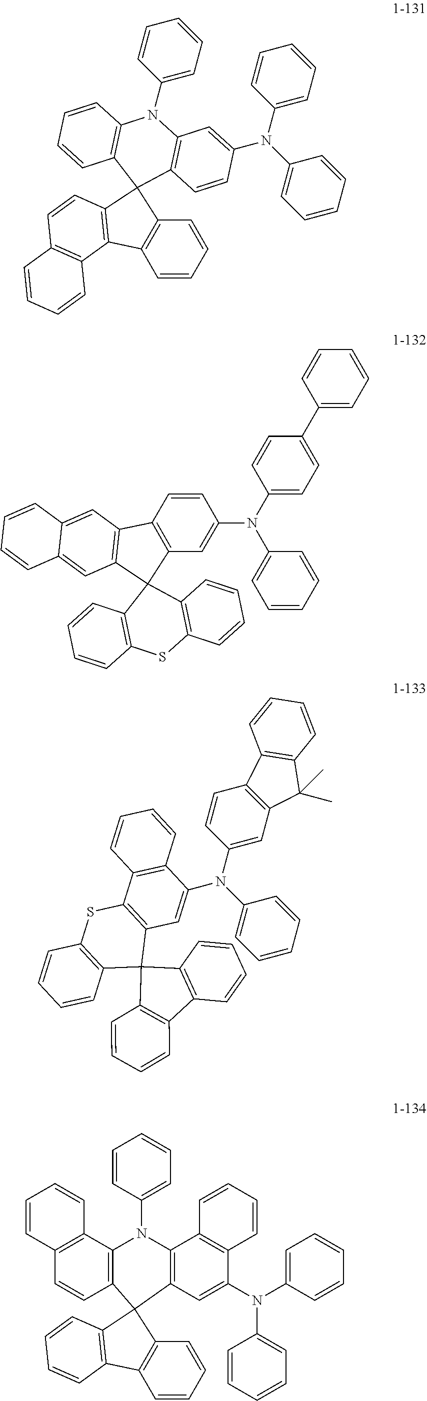 [SMILES: c1ccc(N(c2ccccc2)c2ccc3c(c2)N(c2ccccc2)c2ccccc2C32c3ccccc3-c3c2ccc2ccccc32)cc1 ; c1ccc(-c2ccc(N(c3ccccc3)c3ccc4c(c3)C3(c5ccccc5Sc5ccccc53)c3cc5ccccc5cc3-4)cc2)cc1 ; CC1(C)c2ccccc2-c2ccc(N(c3ccccc3)c3cc4c(c5ccccc35)Sc3ccccc3C43c4ccccc4-c4ccccc43)cc21 ; c1ccc(N(c2ccccc2)c2cc3c(c4ccccc24)N(c2ccccc2)c2c(ccc4ccccc24)C32c3ccccc3-c3ccccc32)cc1]

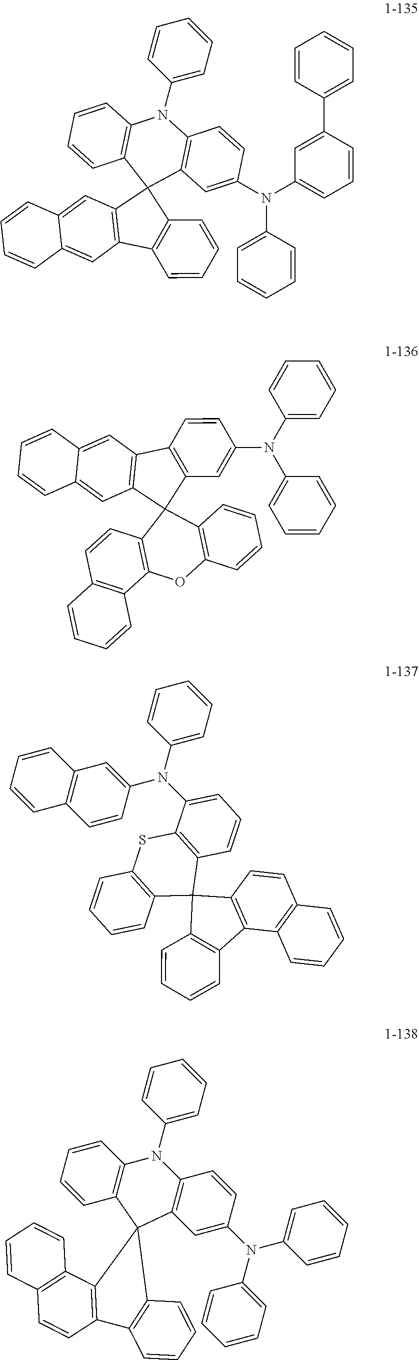 [SMILES: c1ccc(-c2cccc(N(c3ccccc3)c3ccc4c(c3)C3(c5ccccc5-c5cc6ccccc6cc53)c3ccccc3N4c3ccccc3)c2)cc1 ; c1ccc(N(c2ccccc2)c2ccc3c(c2)C2(c4ccccc4Oc4c2ccc2ccccc42)c2cc4ccccc4cc2-3)cc1 ; c1ccc(N(c2ccc3ccccc3c2)c2cccc3c2Sc2ccccc2C32c3ccccc3-c3c2ccc2ccccc32)cc1 ; c1ccc(N(c2ccccc2)c2ccc3c(c2)C2(c4ccccc4-c4ccc5ccccc5c42)c2ccccc2N3c2ccccc2)cc1]

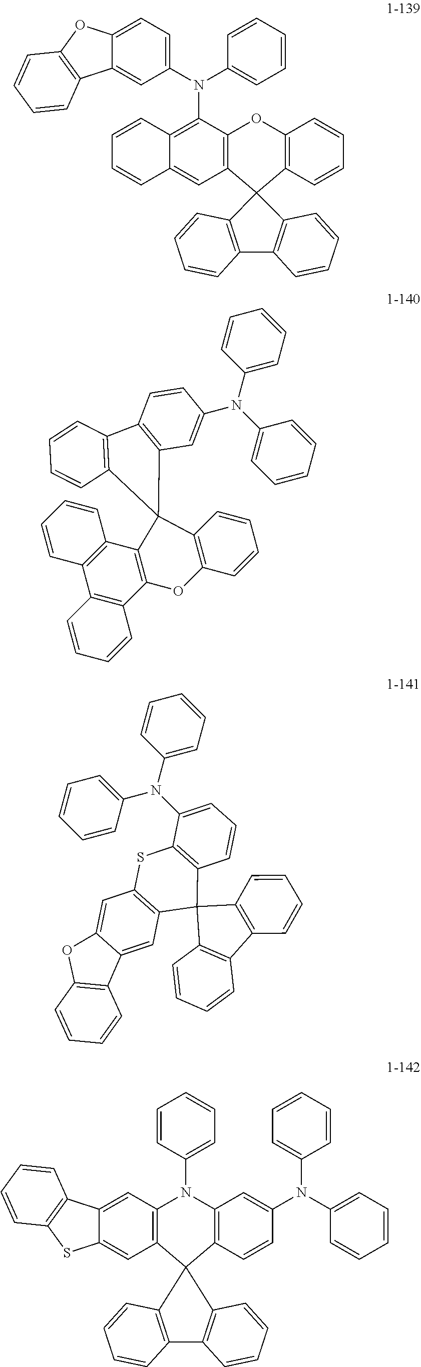

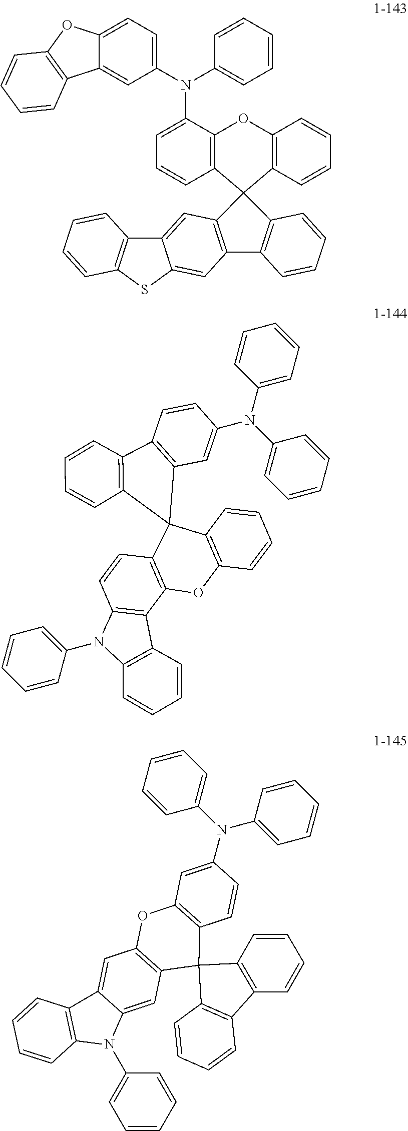

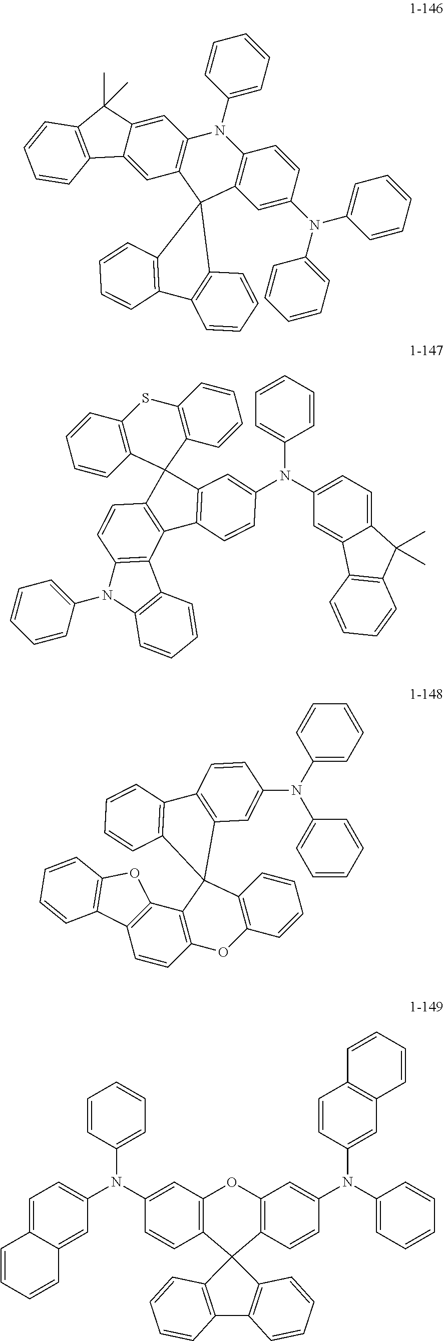 [SMILES: CC1(C)c2ccccc2-c2cc3c(cc21)N(c1ccccc1)c1ccc(N(c2ccccc2)c2ccccc2)cc1C31c2ccccc2-c2ccccc21 ; CC1(C)c2ccccc2-c2cc(N(c3ccccc3)c3ccc4c(c3)C3(c5ccccc5Sc5ccccc53)c3ccc5c(c3-4)c3ccccc3n5-c3ccccc3)ccc21 ; c1ccc(N(c2ccccc2)c2ccc3c(c2)C2(c4ccccc4Oc4ccc5c(oc6ccccc65)c42)c2ccccc2-3)cc1 ; c1ccc(N(c2ccc3c(c2)Oc2cc(N(c4ccccc4)c4ccc5ccccc5c4)ccc2C32c3ccccc3-c3ccccc32)c2ccc3ccccc3c2)cc1]

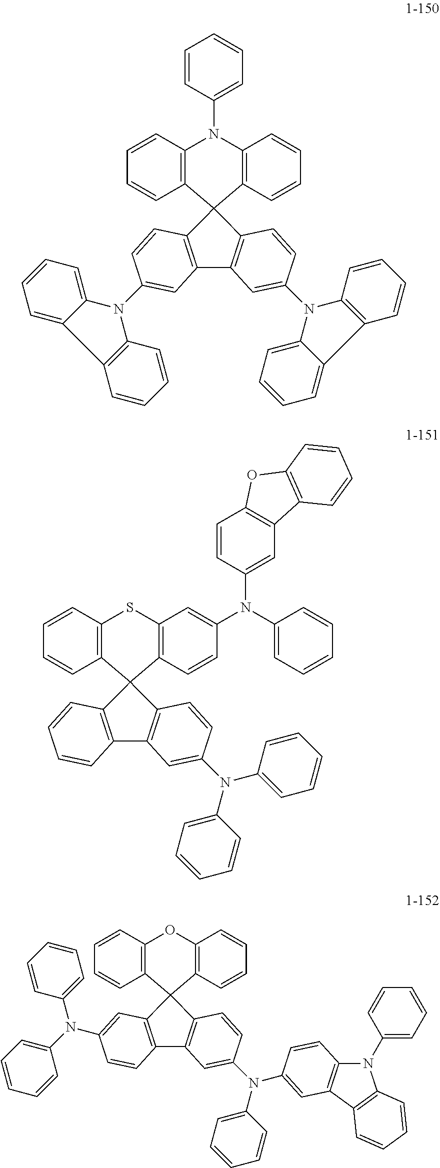

7. The organic electronic element of claim 1, wherein the compound represented by Formula 2 is represented by any one of Formulas 2-1 to 2-3: 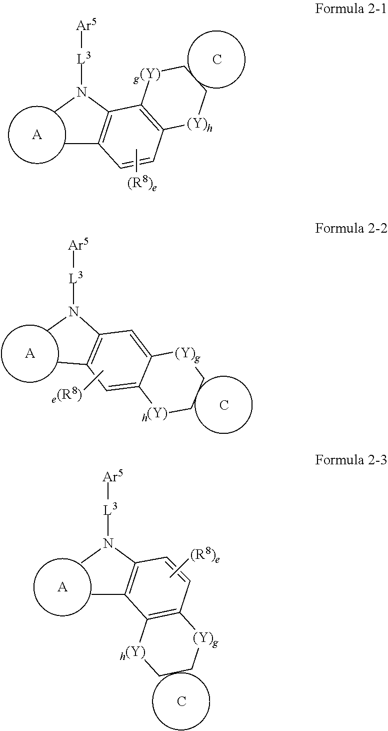
wherein
1) Ring A, Ring C, R⁸, L³, Ar⁵ and Y are the same as defined in claim 1, and
2) e is an integer of 0 to 2, g and h are each an integer of 0 or 1, provided that g+h is 1.

8. The organic electronic element of claim 1, wherein the host compound represented by Formula 2 is represented by any one of Formulas 2-4 to 2-27: 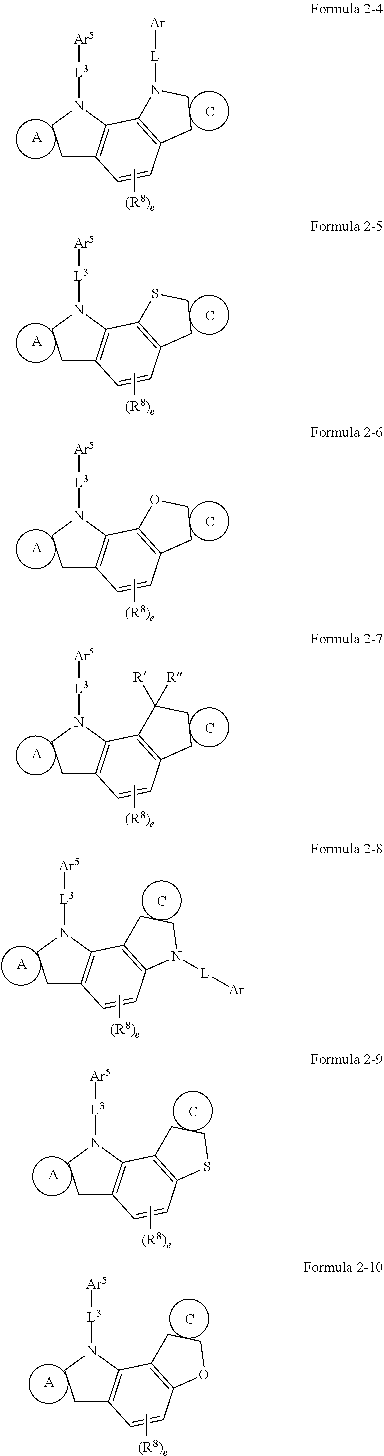 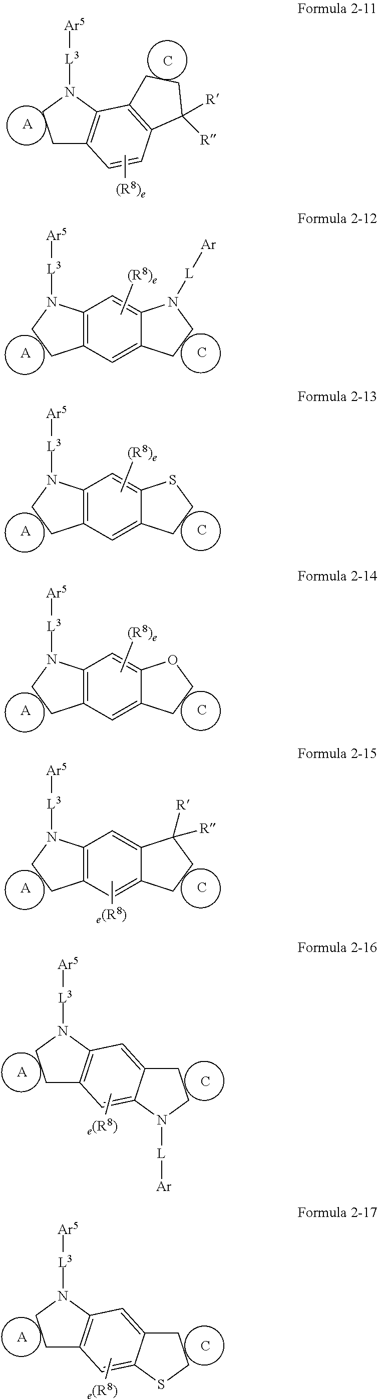 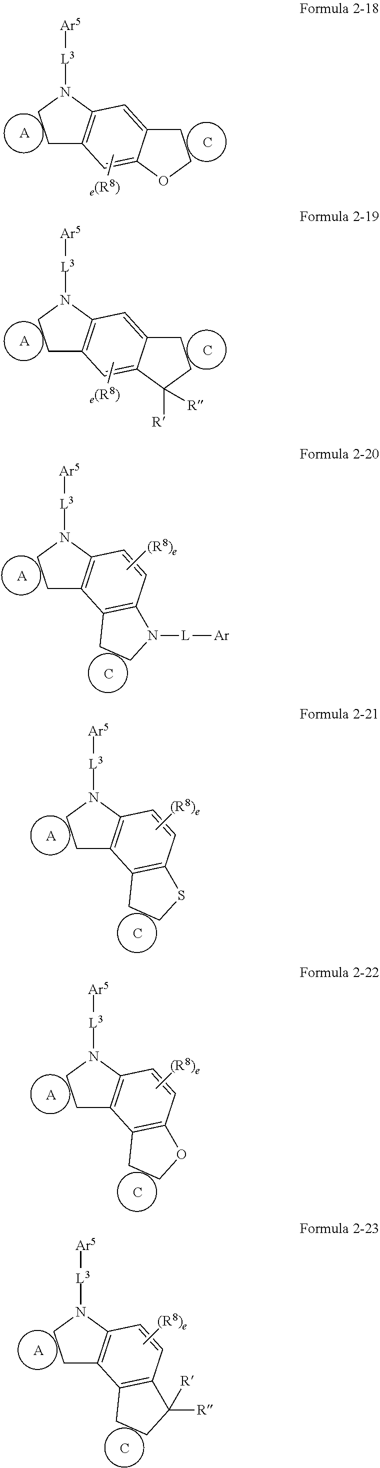 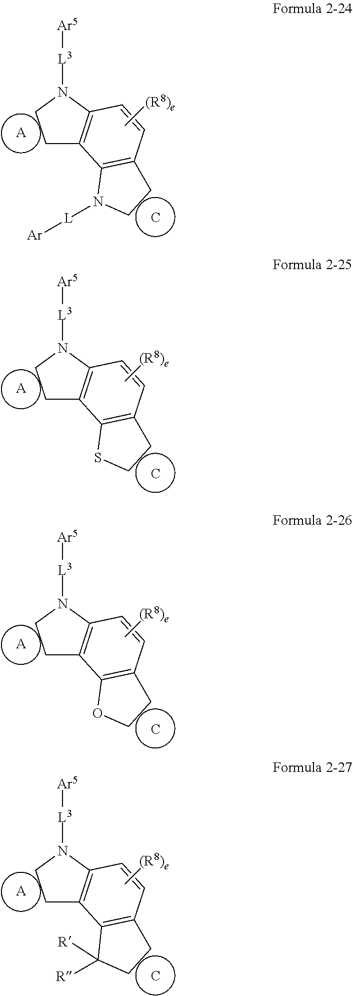
wherein:
1) Ring A, Ring C, R⁸, L³ and Ar⁵ are the same as defined in claim 1,
2) e is 0 to 2,
3) R′ and R″ are the same as the definition of R¹ in claim 1,
4) L is the same as the definition of L¹ in claim 1,
5) Ar is the same as the definition of Ar¹ in claim 1.

9. The organic electronic element of claim 1, wherein at least one of R⁶ to R⁹ and Ar⁵ is represented by any one of Formulas A-1 to A-6: 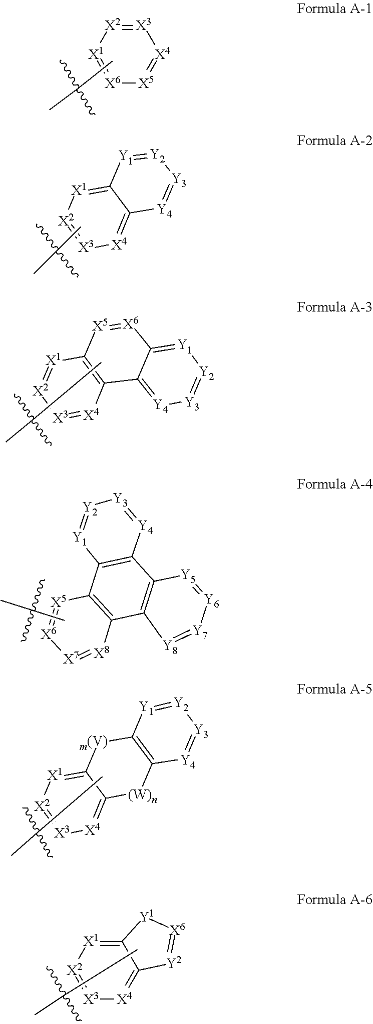
wherein:
1) X¹, X², X³, X⁴, X⁵, X⁶, X⁷ and X⁶ are each independently C, C(R₁) or N,
2) Y₁, Y₂, Y₃, Y₄, Y₅, Y₆, Y₇ and Y₈ are each independently C(R₁) or N,
3) in Formula A-1, at least one of X¹ to X⁶ is N,
4) in Formula A-2, at least one of X¹ to X⁴ and Y₁ to Y₄ is N,
5) in Formula A-3, at least one of X¹ to X⁶ is N,
6) in Formula A-4, at least one of X⁵ to X⁸ and Y₁ to Y₈ is N,
7) in Formula A-5, at least one of X¹ to X⁴ is N,
8) in Formula A-6, X¹, X², X³, X⁴ and X⁶ are each independently C, C(R₁) or N, Y¹ is O, S, N-L′-Ar′ or CR¹³C¹⁴, Y² is N,
9) V and W are each independently O, S, N-L′-Ar′ or CR¹³C¹⁴,
10) m and n are each independently 0 or 1, provided that at least one of m and n is 1,
11) R₁, R¹³ and R¹⁴ are each independently selected from the group consisting of hydrogen; deuterium; halogen; a C₁-C₂₀ alkyl group; or a silane group unsubstituted or substituted with C₆-C₂₀ aryl group; cyano group; nitro group; C₁-C₂₀ alkylthio group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-C₂₀ aryl group; fluorenyl group; a C₂-C₂₀ heterocyclic group including at least one heteroatom of O, N, S, Si or P; a C₃-C₂₀ aliphatic ring; C₇-C₂₀ arylalkyl group; and C₈-C₂₀ aryl alkenyl group; and adjacent R₁s, adjacent R¹³s and adjacent R¹⁴s may be bonded to each other to form a ring,
12) wherein Ar′ is selected from the group consisting of a C₆-C₂₀ aryl group; fluorenyl group; C₂-C₂₀ heterocyclic group containing at least one heteroatom of O, N, S, Si or P; C₃-C₂₀ aliphatic ring; and combinations thereof, and
13) wherein L′ is each independently selected from the group consisting of a single bond; a C₆-C₂₀ arylene group; a fluorenylene group; a C₂-C₂₀ heterocyclic group containing at least one heteroatom of O, N, S, Si or P; and a C₃-C₂₀ aliphatic ring group.

10. The organic electronic element of claim 1, wherein the compound represented by Formula 2 is any one of the following compounds:

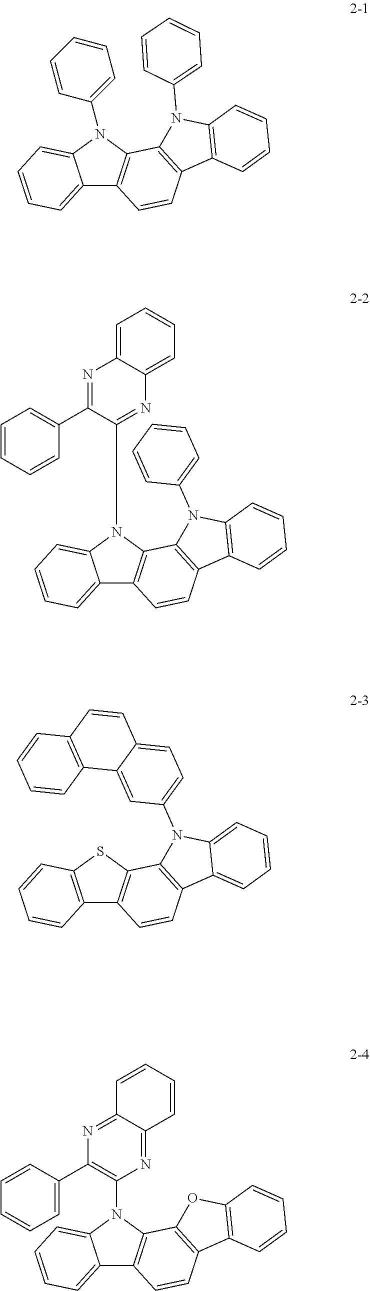 [SMILES: c1ccc(-n2c3ccccc3c3ccc4c5ccccc5n(-c5ccccc5)c4c32)cc1 ; c1ccc(-c2nc3ccccc3nc2-n2c3ccccc3c3ccc4c5ccccc5n(-c5ccccc5)c4c32)cc1 ; c1ccc2c(c1)ccc1ccc(-n3c4ccccc4c4ccc5c6ccccc6sc5c43)cc12 ; c1ccc(-c2nc3ccccc3nc2-n2c3ccccc3c3ccc4c5ccccc5oc4c32)cc1]

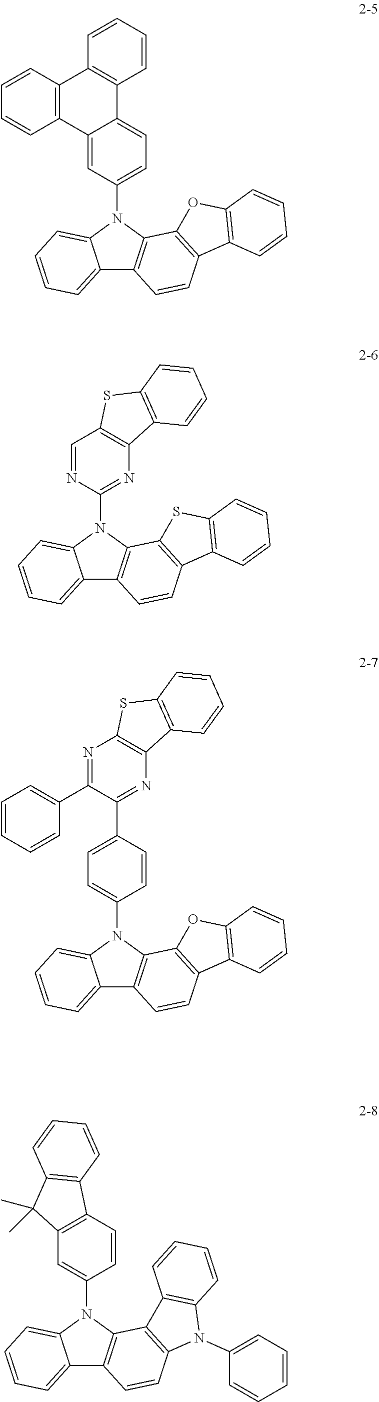 [SMILES: c1ccc2c(c1)oc1c2ccc2c3ccccc3n(-c3ccc4c5ccccc5c5ccccc5c4c3)c21 ; c1ccc2c(c1)sc1c2ccc2c3ccccc3n(-c3ncc4sc5ccccc5c4n3)c21 ; c1ccc(-c2nc3sc4ccccc4c3nc2-c2ccc(-n3c4ccccc4c4ccc5c6ccccc6oc5c43)cc2)cc1 ; CC1(C)c2ccccc2-c2ccc(-n3c4ccccc4c4ccc5c(c6ccccc6n5-c5ccccc5)c43)cc21]

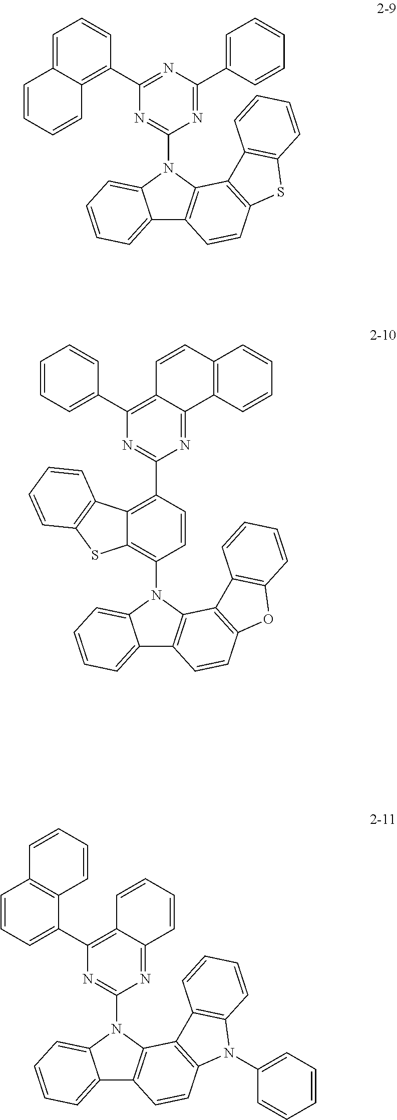 [SMILES: c1ccc(-c2nc(-c3cccc4ccccc34)nc(-n3c4ccccc4c4ccc5sc6ccccc6c5c43)n2)cc1 ; c1ccc(-c2nc(-c3ccc(-n4c5ccccc5c5ccc6oc7ccccc7c6c54)c4sc5ccccc5c34)nc3c2ccc2ccccc23)cc1 ; c1ccc(-n2c3ccccc3c3c2ccc2c4ccccc4n(-c4nc(-c5cccc6ccccc56)c5ccccc5n4)c23)cc1]

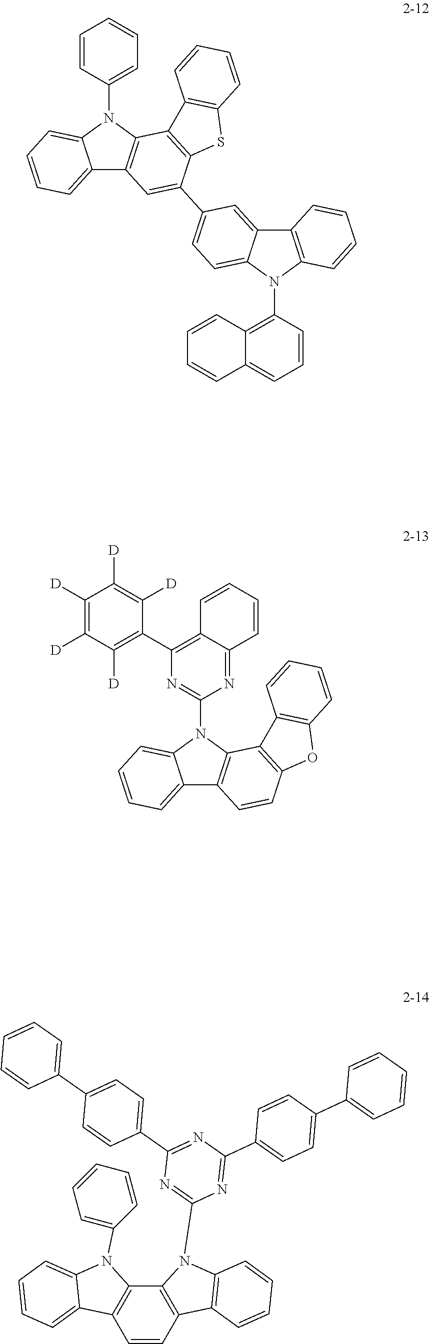 [SMILES: c1ccc(-n2c3ccccc3c3cc(-c4ccc5c(c4)c4ccccc4n5-c4cccc5ccccc45)c4sc5ccccc5c4c32)cc1 ; [2H]c1c([2H])c([2H])c(-c2nc(-n3c4ccccc4c4ccc5oc6ccccc6c5c43)nc3ccccc23)c([2H])c1[2H] ; c1ccc(-c2ccc(-c3nc(-c4ccc(-c5ccccc5)cc4)nc(-n4c5ccccc5c5ccc6c7ccccc7n(-c7ccccc7)c6c54)n3)cc2)cc1]

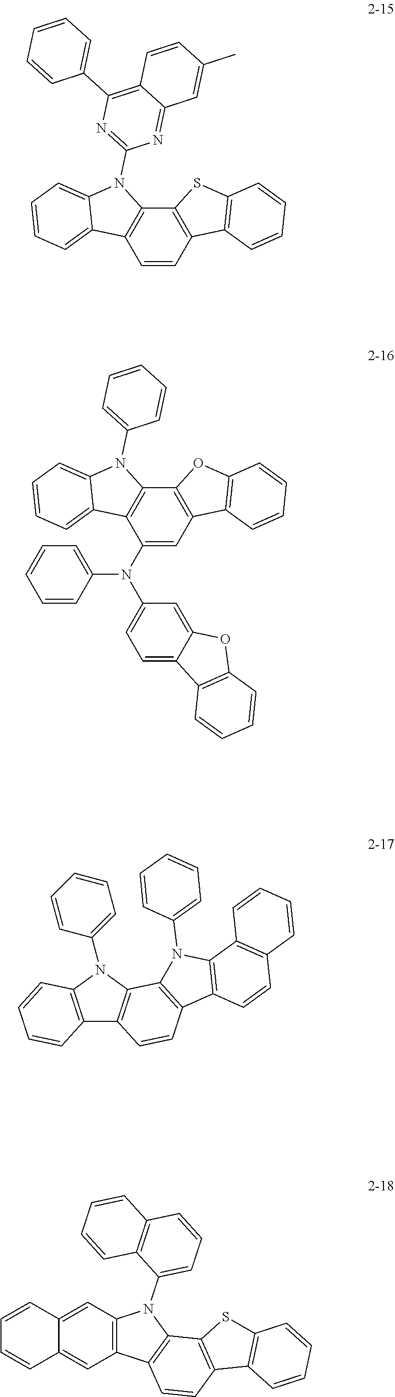 [SMILES: Cc1ccc2c(-c3ccccc3)nc(-n3c4ccccc4c4ccc5c6ccccc6sc5c43)nc2c1 ; c1ccc(N(c2ccc3c(c2)oc2ccccc23)c2cc3c4ccccc4oc3c3c2c2ccccc2n3-c2ccccc2)cc1 ; c1ccc(-n2c3ccccc3c3ccc4c5ccc6ccccc6c5n(-c5ccccc5)c4c32)cc1 ; c1ccc2cc3c(cc2c1)c1ccc2c4ccccc4sc2c1n3-c1cccc2ccccc12]

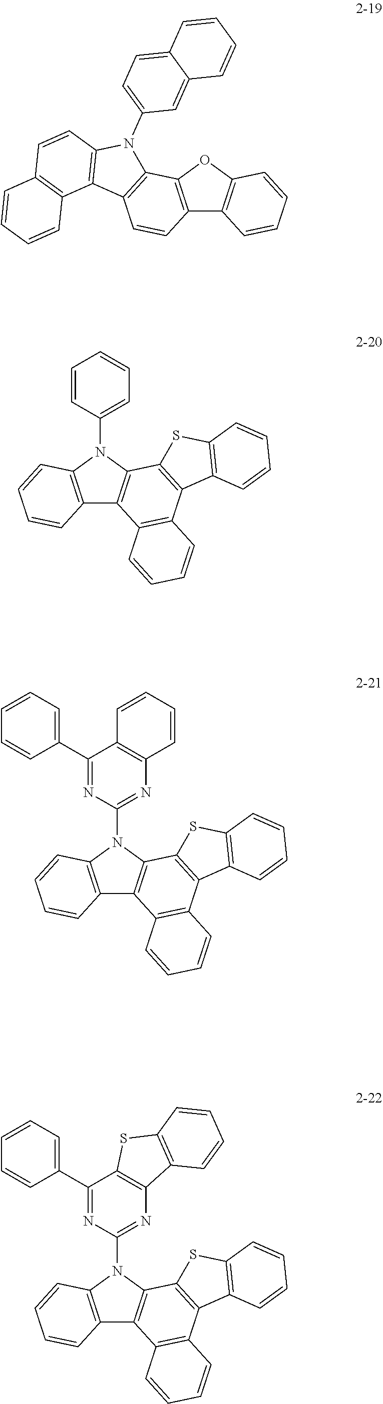 [SMILES: c1ccc2cc(-n3c4ccc5ccccc5c4c4ccc5c6ccccc6oc5c43)ccc2c1 ; c1ccc(-n2c3ccccc3c3c4ccccc4c4c5ccccc5sc4c32)cc1 ; c1ccc(-c2nc(-n3c4ccccc4c4c5ccccc5c5c6ccccc6sc5c43)nc3ccccc23)cc1 ; c1ccc(-c2nc(-n3c4ccccc4c4c5ccccc5c5c6ccccc6sc5c43)nc3c2sc2ccccc23)cc1]

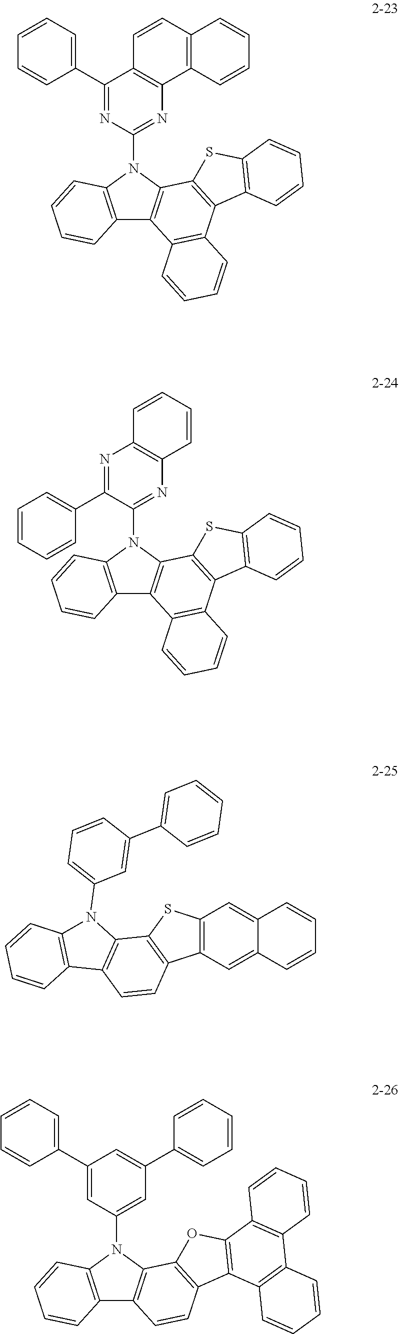 [SMILES: c1ccc(-c2nc(-n3c4ccccc4c4c5ccccc5c5c6ccccc6sc5c43)nc3c2ccc2ccccc23)cc1 ; c1ccc(-c2nc3ccccc3nc2-n2c3ccccc3c3c4ccccc4c4c5ccccc5sc4c32)cc1 ; c1ccc(-c2cccc(-n3c4ccccc4c4ccc5c6cc7ccccc7cc6sc5c43)c2)cc1 ; c1ccc(-c2cc(-c3ccccc3)cc(-n3c4ccccc4c4ccc5c(oc6c7ccccc7c7ccccc7c56)c43)c2)cc1]

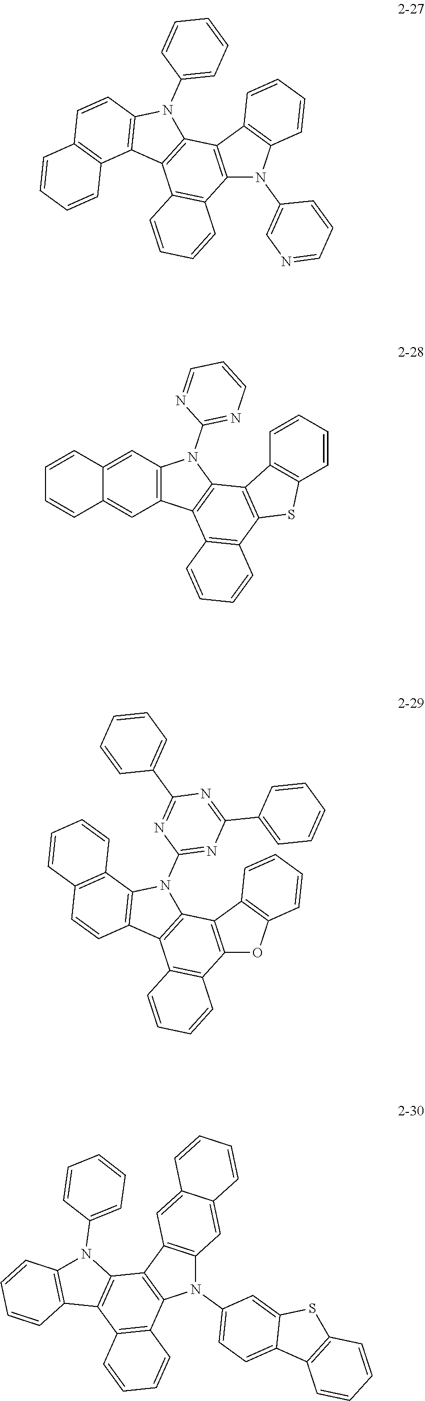 [SMILES: c1ccc(-n2c3ccc4ccccc4c3c3c4ccccc4c4c(c5ccccc5n4-c4cccnc4)c32)cc1 ; c1cnc(-n2c3cc4ccccc4cc3c3c4ccccc4c4sc5ccccc5c4c32)nc1 ; c1ccc(-c2nc(-c3ccccc3)nc(-n3c4c5ccccc5ccc4c4c5ccccc5c5oc6ccccc6c5c43)n2)cc1 ; c1ccc(-n2c3ccccc3c3c4ccccc4c4c(c5cc6ccccc6cc5n4-c4ccc5c(c4)sc4ccccc45)c32)cc1]

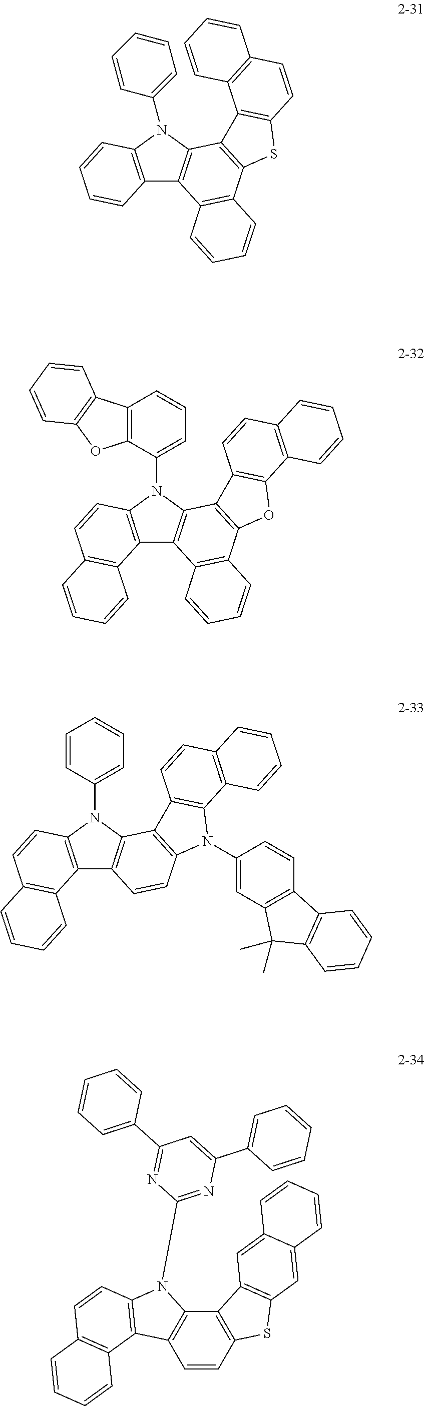 [SMILES: c1ccc(-n2c3ccccc3c3c4ccccc4c4sc5ccc6ccccc6c5c4c32)cc1 ; c1ccc2c(c1)ccc1c2oc2c3ccccc3c3c4c5ccccc5ccc4n(-c4cccc5c4oc4ccccc45)c3c12 ; CC1(C)c2ccccc2-c2ccc(-n3c4ccc5c6c7ccccc7ccc6n(-c6ccccc6)c5c4c4ccc5ccccc5c43)cc21 ; c1ccc(-c2cc(-c3ccccc3)nc(-n3c4ccc5ccccc5c4c4ccc5sc6cc7ccccc7cc6c5c43)n2)cc1]

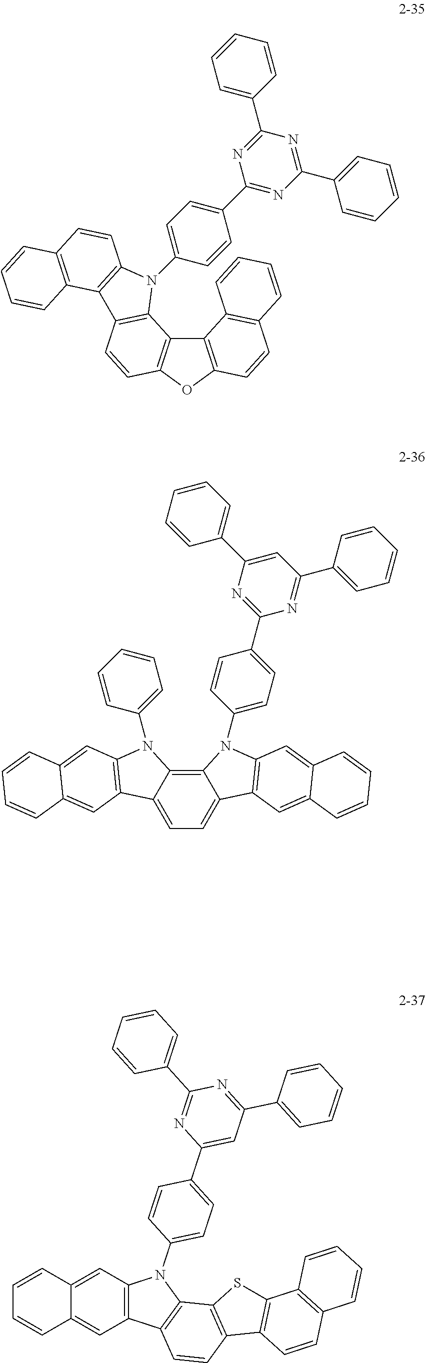 [SMILES: c1ccc(-c2nc(-c3ccccc3)nc(-c3ccc(-n4c5ccc6ccccc6c5c5ccc6oc7ccc8ccccc8c7c6c54)cc3)n2)cc1 ; c1ccc(-c2cc(-c3ccccc3)nc(-c3ccc(-n4c5cc6ccccc6cc5c5ccc6c7cc8ccccc8cc7n(-c7ccccc7)c6c54)cc3)n2)cc1 ; c1ccc(-c2cc(-c3ccc(-n4c5cc6ccccc6cc5c5ccc6c7ccc8ccccc8c7sc6c54)cc3)nc(-c3ccccc3)n2)cc1]

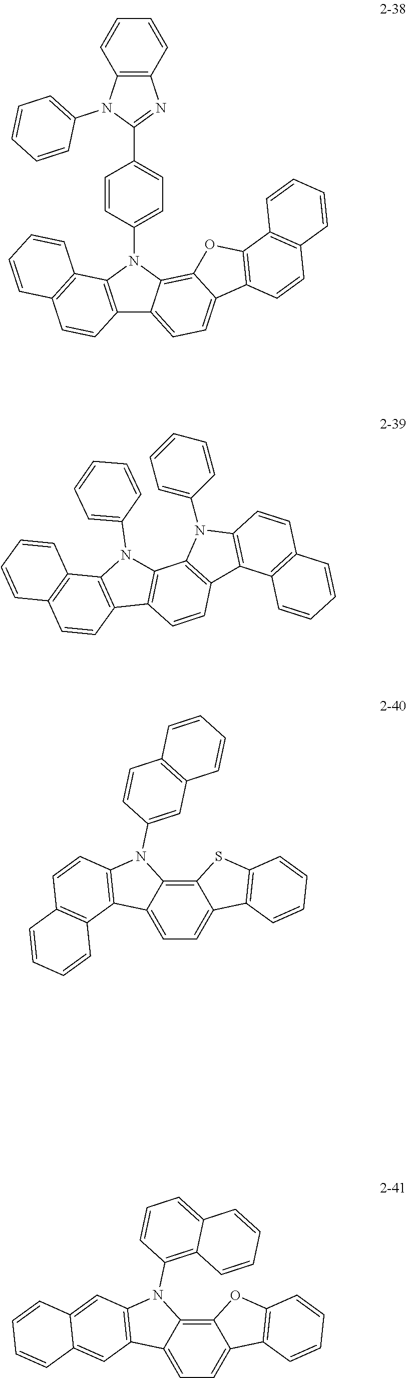 [SMILES: c1ccc(-n2c(-c3ccc(-n4c5c6ccccc6ccc5c5ccc6c7ccc8ccccc8c7oc6c54)cc3)nc3ccccc32)cc1 ; c1ccc(-n2c3ccc4ccccc4c3c3ccc4c5ccc6ccccc6c5n(-c5ccccc5)c4c32)cc1 ; c1ccc2cc(-n3c4ccc5ccccc5c4c4ccc5c6ccccc6sc5c43)ccc2c1 ; c1ccc2cc3c(cc2c1)c1ccc2c4ccccc4oc2c1n3-c1cccc2ccccc12]

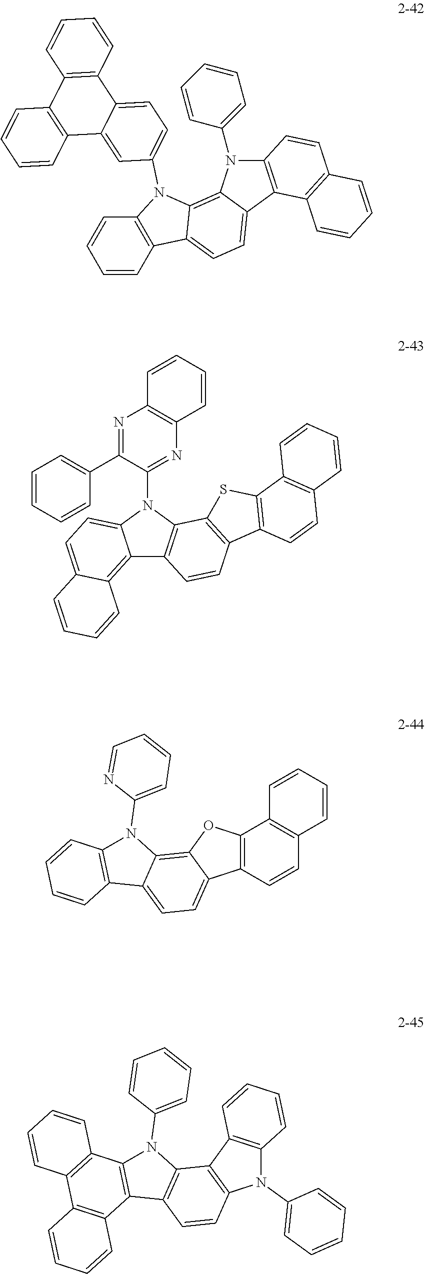 [SMILES: c1ccc(-n2c3ccc4ccccc4c3c3ccc4c5ccccc5n(-c5ccc6c7ccccc7c7ccccc7c6c5)c4c32)cc1 ; c1ccc(-c2nc3ccccc3nc2-n2c3ccc4ccccc4c3c3ccc4c5ccc6ccccc6c5sc4c32)cc1 ; c1ccc(-n2c3ccccc3c3ccc4c5ccc6ccccc6c5oc4c32)nc1 ; c1ccc(-n2c3ccccc3c3c2ccc2c4c5ccccc5c5ccccc5c4n(-c4ccccc4)c23)cc1]

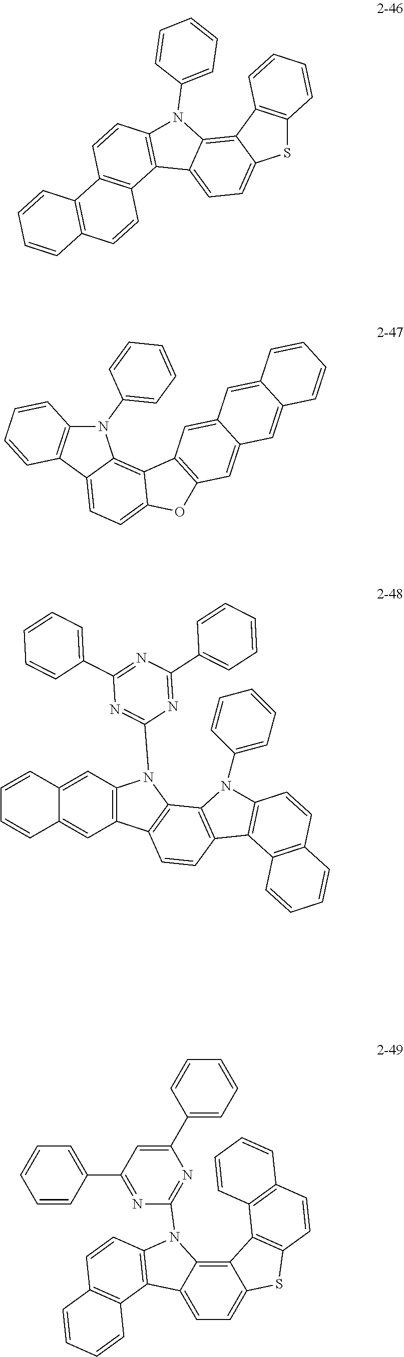 [SMILES: c1ccc(-n2c3ccc4c5ccccc5ccc4c3c3ccc4sc5ccccc5c4c32)cc1 ; c1ccc(-n2c3ccccc3c3ccc4oc5cc6cc7ccccc7cc6cc5c4c32)cc1 ; c1ccc(-c2nc(-c3ccccc3)nc(-n3c4cc5ccccc5cc4c4ccc5c6c7ccccc7ccc6n(-c6ccccc6)c5c43)n2)cc1 ; c1ccc(-c2cc(-c3ccccc3)nc(-n3c4ccc5ccccc5c4c4ccc5sc6ccc7ccccc7c6c5c43)n2)cc1]

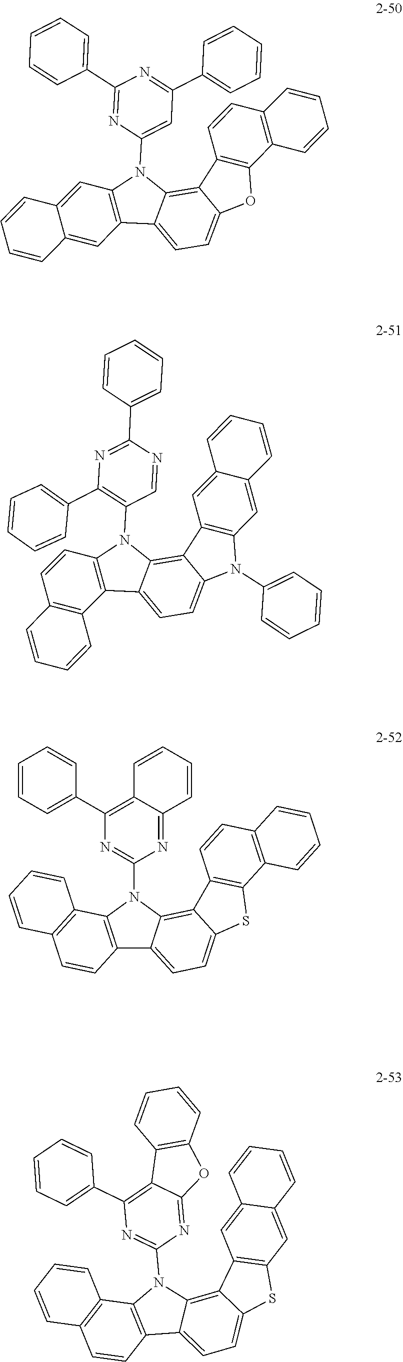 [SMILES: c1ccc(-c2cc(-n3c4cc5ccccc5cc4c4ccc5oc6c7ccccc7ccc6c5c43)nc(-c3ccccc3)n2)cc1 ; c1ccc(-c2ncc(-n3c4ccc5ccccc5c4c4ccc5c(c6cc7ccccc7cc6n5-c5ccccc5)c43)c(-c3ccccc3)n2)cc1 ; c1ccc(-c2nc(-n3c4c5ccccc5ccc4c4ccc5sc6c7ccccc7ccc6c5c43)nc3ccccc23)cc1 ; c1ccc(-c2nc(-n3c4c5ccccc5ccc4c4ccc5sc6cc7ccccc7cc6c5c43)nc3oc4ccccc4c23)cc1]

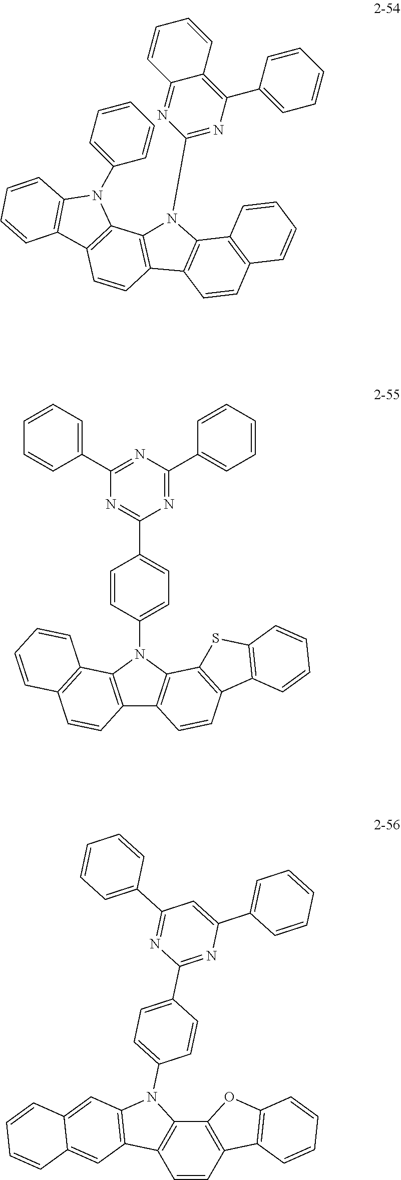 [SMILES: c1ccc(-c2nc(-n3c4c5ccccc5ccc4c4ccc5c6ccccc6n(-c6ccccc6)c5c43)nc3ccccc23)cc1 ; c1ccc(-c2nc(-c3ccccc3)nc(-c3ccc(-n4c5c6ccccc6ccc5c5ccc6c7ccccc7sc6c54)cc3)n2)cc1 ; c1ccc(-c2cc(-c3ccccc3)nc(-c3ccc(-n4c5cc6ccccc6cc5c5ccc6c7ccccc7oc6c54)cc3)n2)cc1]

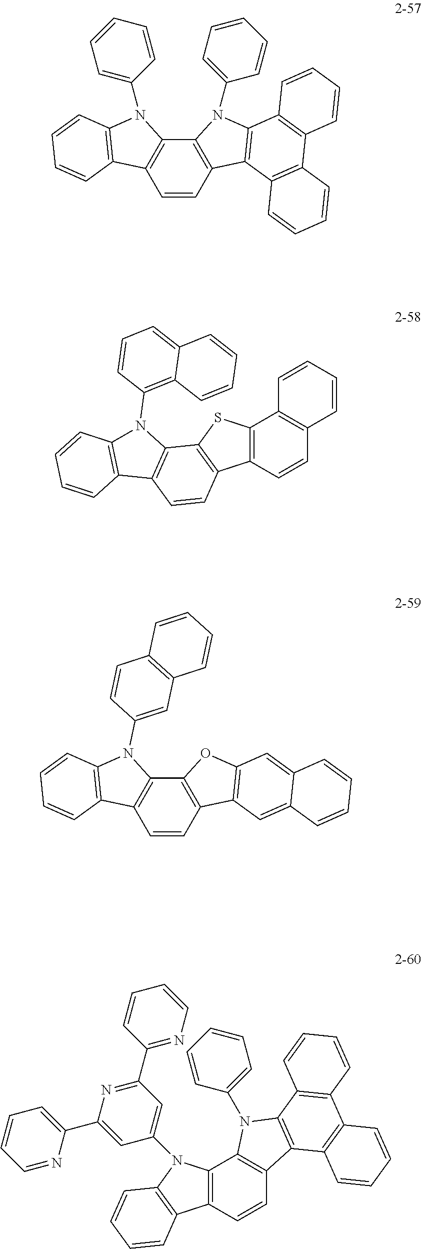 [SMILES: c1ccc(-n2c3ccccc3c3ccc4c5c6ccccc6c6ccccc6c5n(-c5ccccc5)c4c32)cc1 ; c1ccc2c(-n3c4ccccc4c4ccc5c6ccc7ccccc7c6sc5c43)cccc2c1 ; c1ccc2cc(-n3c4ccccc4c4ccc5c6cc7ccccc7cc6oc5c43)ccc2c1 ; c1ccc(-n2c3c4ccccc4c4ccccc4c3c3ccc4c5ccccc5n(-c5cc(-c6ccccn6)nc(-c6ccccn6)c5)c4c32)cc1]

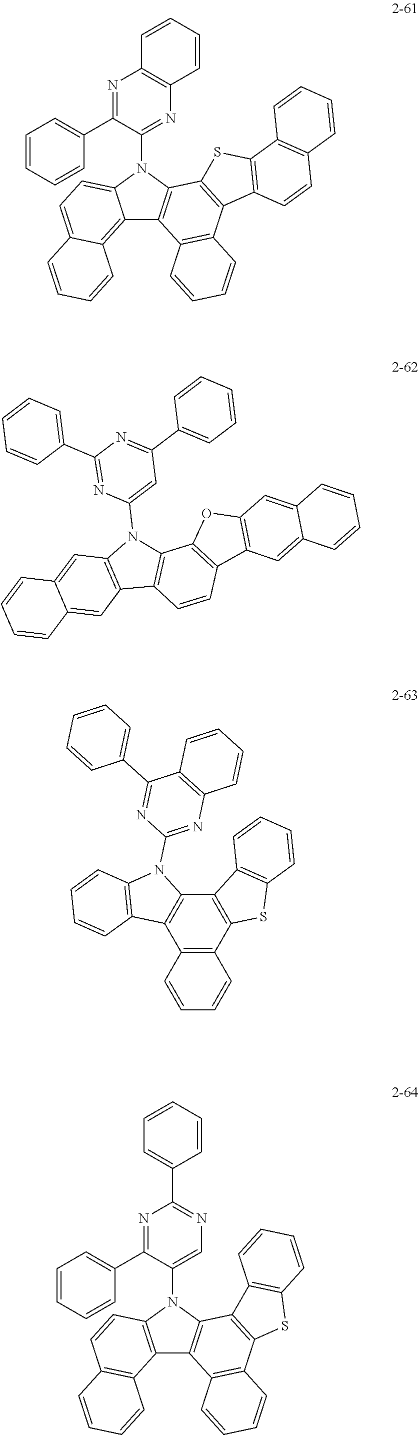 [SMILES: c1ccc(-c2nc3ccccc3nc2-n2c3ccc4ccccc4c3c3c4ccccc4c4c5ccc6ccccc6c5sc4c32)cc1 ; c1ccc(-c2cc(-n3c4cc5ccccc5cc4c4ccc5c6cc7ccccc7cc6oc5c43)nc(-c3ccccc3)n2)cc1 ; c1ccc(-c2nc(-n3c4ccccc4c4c5ccccc5c5sc6ccccc6c5c43)nc3ccccc23)cc1 ; c1ccc(-c2ncc(-n3c4ccc5ccccc5c4c4c5ccccc5c5sc6ccccc6c5c43)c(-c3ccccc3)n2)cc1]

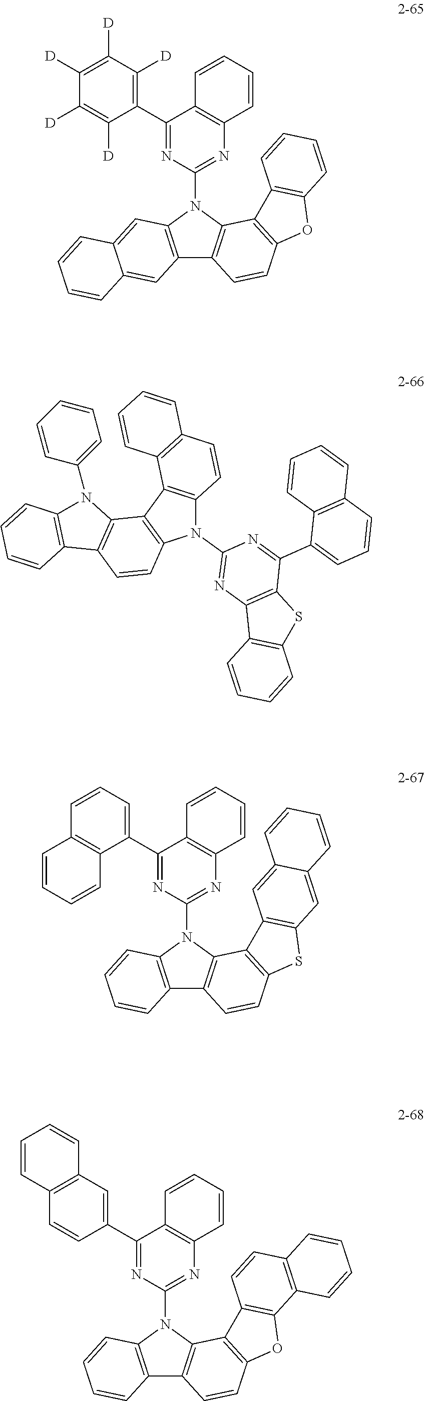 [SMILES: [2H]c1c([2H])c([2H])c(-c2nc(-n3c4cc5ccccc5cc4c4ccc5oc6ccccc6c5c43)nc3ccccc23)c([2H])c1[2H] ; c1ccc(-n2c3ccccc3c3ccc4c(c5c6ccccc6ccc5n4-c4nc(-c5cccc6ccccc56)c5sc6ccccc6c5n4)c32)cc1 ; c1ccc2cc3c(cc2c1)sc1ccc2c4ccccc4n(-c4nc(-c5cccc6ccccc56)c5ccccc5n4)c2c13 ; c1ccc2cc(-c3nc(-n4c5ccccc5c5ccc6oc7c8ccccc8ccc7c6c54)nc4ccccc34)ccc2c1]

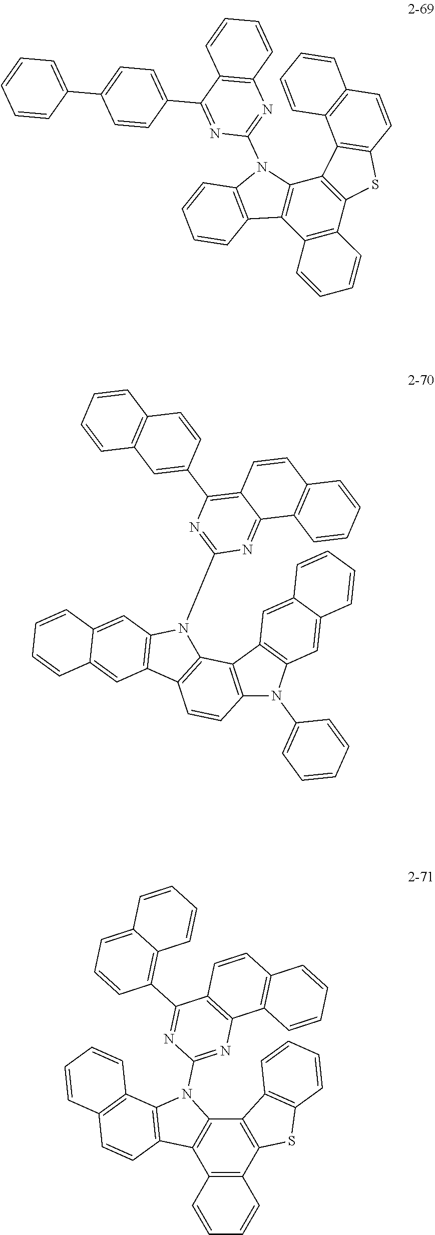 [SMILES: c1ccc(-c2ccc(-c3nc(-n4c5ccccc5c5c6ccccc6c6sc7ccc8ccccc8c7c6c54)nc4ccccc34)cc2)cc1 ; c1ccc(-n2c3cc4ccccc4cc3c3c2ccc2c4cc5ccccc5cc4n(-c4nc(-c5ccc6ccccc6c5)c5ccc6ccccc6c5n4)c23)cc1 ; c1ccc2c(-c3nc(-n4c5c6ccccc6ccc5c5c6ccccc6c6sc7ccccc7c6c54)nc4c3ccc3ccccc34)cccc2c1]

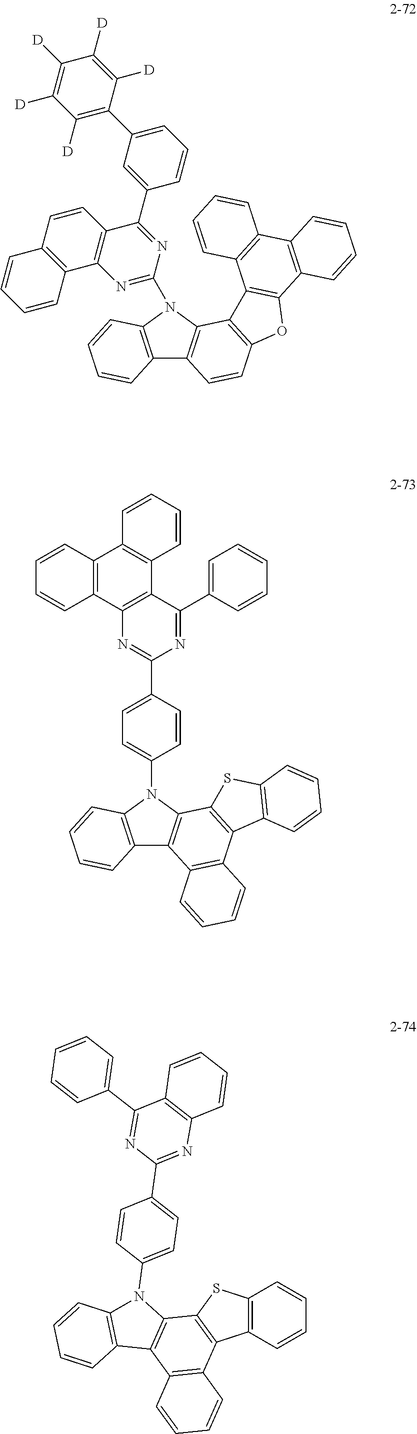 [SMILES: [2H]c1c([2H])c([2H])c(-c2cccc(-c3nc(-n4c5ccccc5c5ccc6oc7c8ccccc8c8ccccc8c7c6c54)nc4c3ccc3ccccc34)c2)c([2H])c1[2H] ; c1ccc(-c2nc(-c3ccc(-n4c5ccccc5c5c6ccccc6c6c7ccccc7sc6c54)cc3)nc3c4ccccc4c4ccccc4c23)cc1 ; c1ccc(-c2nc(-c3ccc(-n4c5ccccc5c5c6ccccc6c6c7ccccc7sc6c54)cc3)nc3ccccc23)cc1]

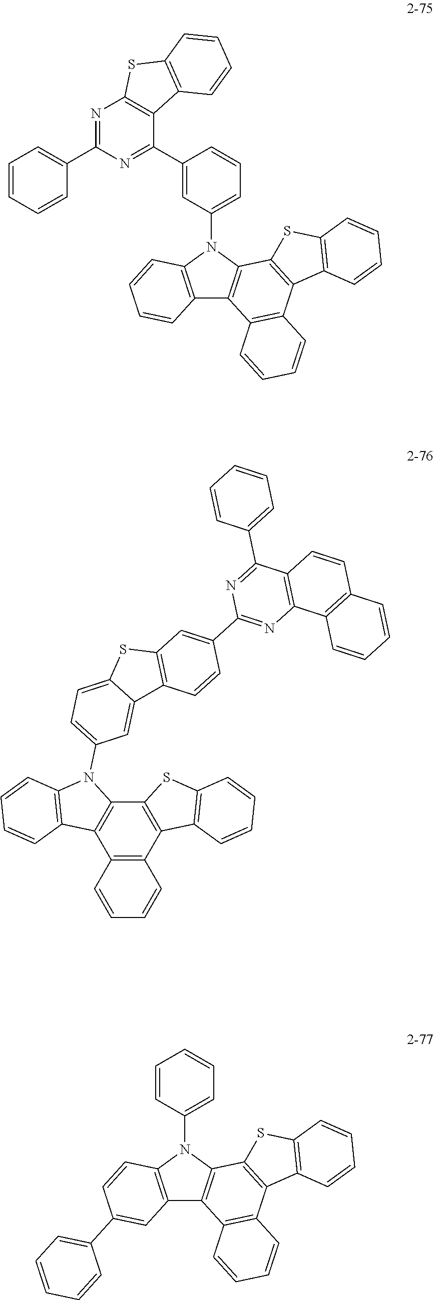 [SMILES: c1ccc(-c2nc(-c3cccc(-n4c5ccccc5c5c6ccccc6c6c7ccccc7sc6c54)c3)c3c(n2)sc2ccccc23)cc1 ; c1ccc(-c2nc(-c3ccc4c(c3)sc3ccc(-n5c6ccccc6c6c7ccccc7c7c8ccccc8sc7c65)cc34)nc3c2ccc2ccccc23)cc1 ; c1ccc(-c2ccc3c(c2)c2c4ccccc4c4c5ccccc5sc4c2n3-c2ccccc2)cc1]

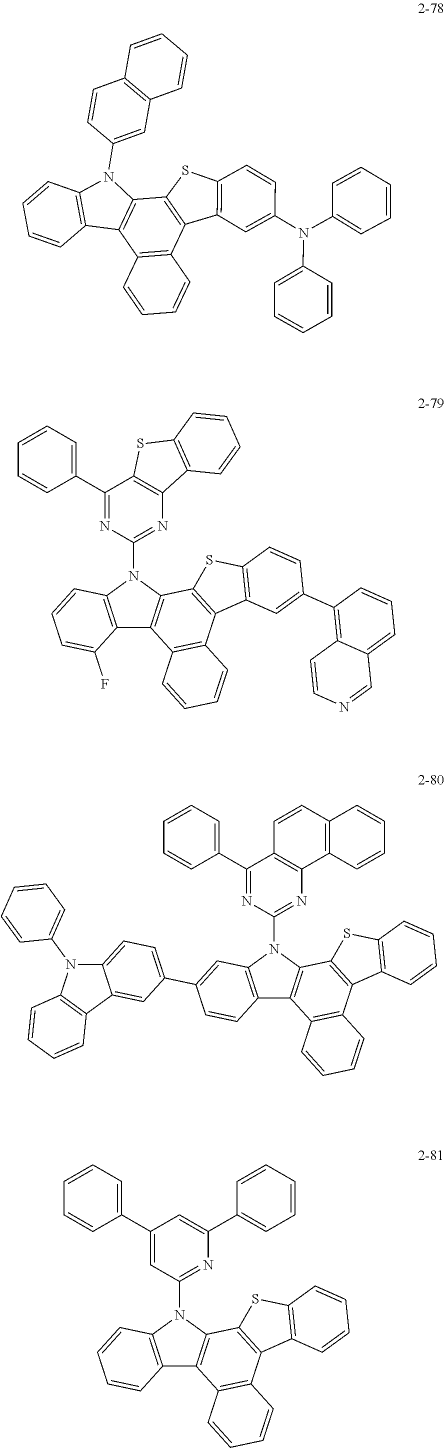 [SMILES: c1ccc(N(c2ccccc2)c2ccc3sc4c(c3c2)c2ccccc2c2c3ccccc3n(-c3ccc5ccccc5c3)c42)cc1 ; Fc1cccc2c1c1c3ccccc3c3c4cc(-c5cccc6cnccc56)ccc4sc3c1n2-c1nc(-c2ccccc2)c2sc3ccccc3c2n1 ; c1ccc(-c2nc(-n3c4cc(-c5ccc6c(c5)c5ccccc5n6-c5ccccc5)ccc4c4c5ccccc5c5c6ccccc6sc5c43)nc3c2ccc2ccccc23)cc1 ; c1ccc(-c2cc(-c3ccccc3)nc(-n3c4ccccc4c4c5ccccc5c5c6ccccc6sc5c43)c2)cc1]

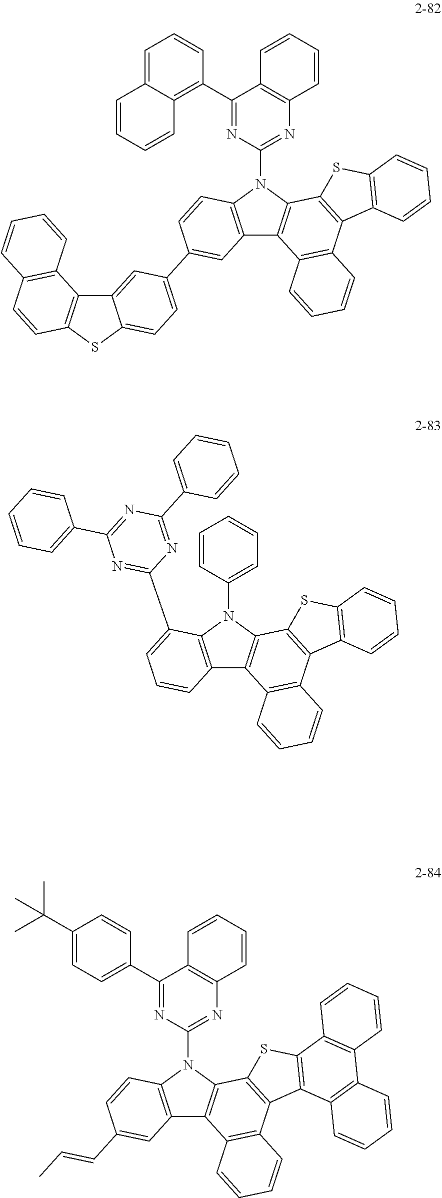 [SMILES: c1ccc2c(-c3nc(-n4c5ccc(-c6ccc7sc8ccc9ccccc9c8c7c6)cc5c5c6ccccc6c6c7ccccc7sc6c54)nc4ccccc34)cccc2c1 ; c1ccc(-c2nc(-c3ccccc3)nc(-c3cccc4c5c6ccccc6c6c7ccccc7sc6c5n(-c5ccccc5)c34)n2)cc1 ; C/C=C/c1ccc2c(c1)c1c3ccccc3c3c(sc4c5ccccc5c5ccccc5c43)c1n2-c1nc(-c2ccc(C(C)(C)C)cc2)c2ccccc2n1]

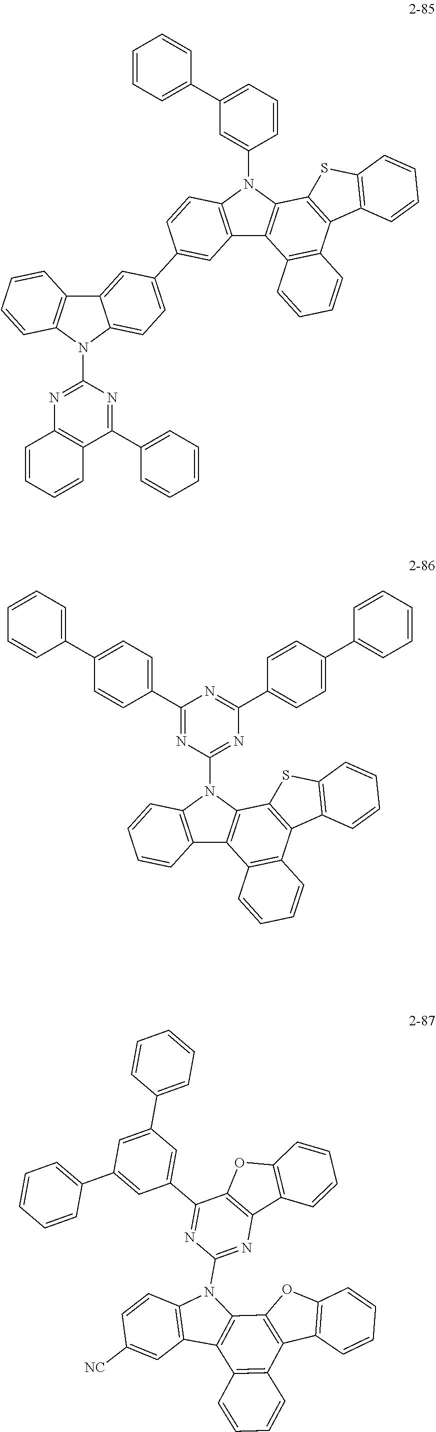 [SMILES: c1ccc(-c2cccc(-n3c4ccc(-c5ccc6c(c5)c5ccccc5n6-c5nc(-c6ccccc6)c6ccccc6n5)cc4c4c5ccccc5c5c6ccccc6sc5c43)c2)cc1 ; c1ccc(-c2ccc(-c3nc(-c4ccc(-c5ccccc5)cc4)nc(-n4c5ccccc5c5c6ccccc6c6c7ccccc7sc6c54)n3)cc2)cc1 ; N#Cc1ccc2c(c1)c1c3ccccc3c3c4ccccc4oc3c1n2-c1nc(-c2cc(-c3ccccc3)cc(-c3ccccc3)c2)c2oc3ccccc3c2n1]

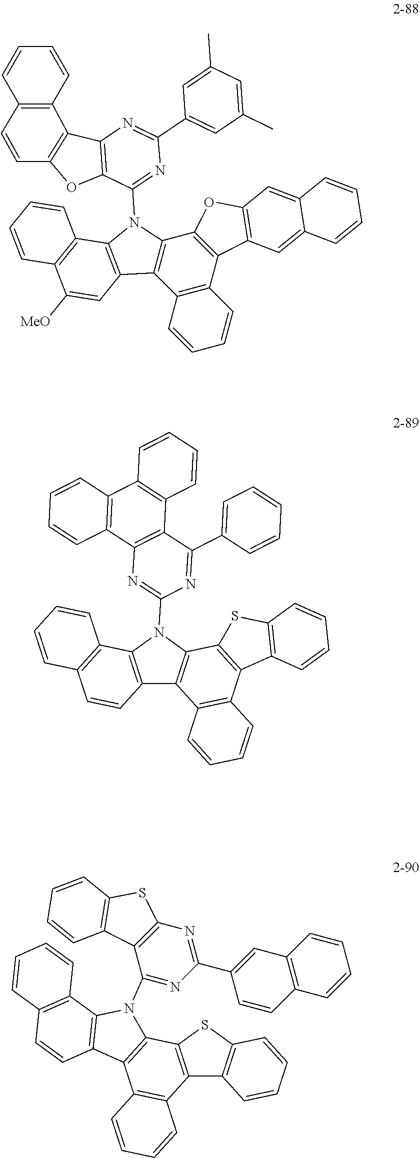 [SMILES: COc1cc2c3c4ccccc4c4c5cc6ccccc6cc5oc4c3n(-c3nc(-c4cc(C)cc(C)c4)nc4c3oc3ccc5ccccc5c34)c2c2ccccc12 ; c1ccc(-c2nc(-n3c4c5ccccc5ccc4c4c5ccccc5c5c6ccccc6sc5c43)nc3c4ccccc4c4ccccc4c23)cc1 ; c1ccc2cc(-c3nc(-n4c5c6ccccc6ccc5c5c6ccccc6c6c7ccccc7sc6c54)c4c(n3)sc3ccccc34)ccc2c1]

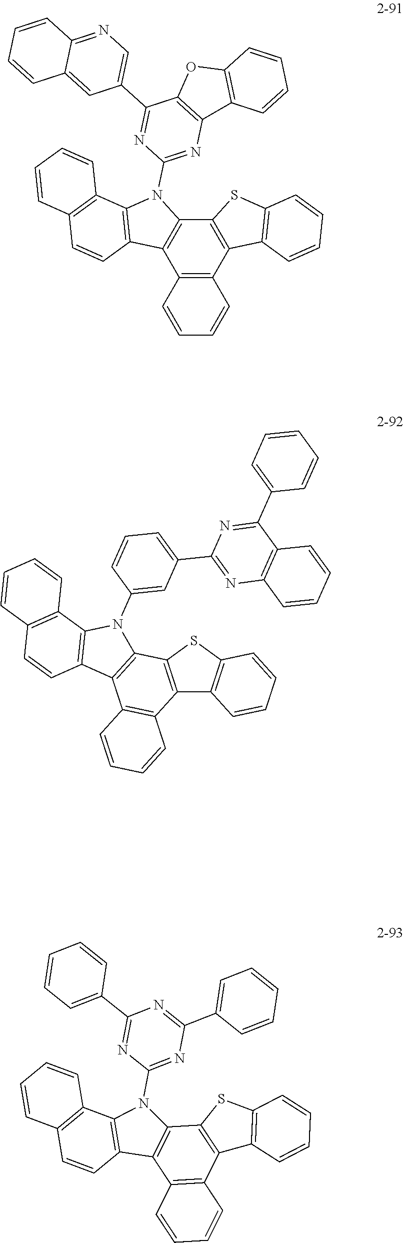 [SMILES: c1ccc2ncc(-c3nc(-n4c5c6ccccc6ccc5c5c6ccccc6c6c7ccccc7sc6c54)nc4c3oc3ccccc34)cc2c1 ; c1ccc(-c2nc(-c3cccc(-n4c5c6ccccc6ccc5c5c6ccccc6c6c7ccccc7sc6c54)c3)nc3ccccc23)cc1 ; c1ccc(-c2nc(-c3ccccc3)nc(-n3c4c5ccccc5ccc4c4c5ccccc5c5c6ccccc6sc5c43)n2)cc1]

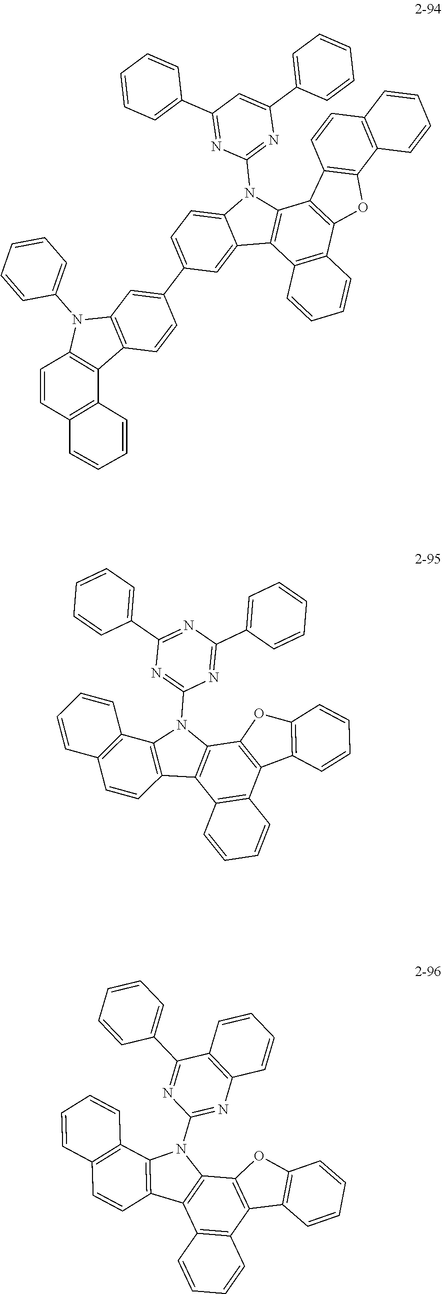 [SMILES: c1ccc(-c2cc(-c3ccccc3)nc(-n3c4ccc(-c5ccc6c7c8ccccc8ccc7n(-c7ccccc7)c6c5)cc4c4c5ccccc5c5oc6c7ccccc7ccc6c5c43)n2)cc1 ; c1ccc(-c2nc(-c3ccccc3)nc(-n3c4c5ccccc5ccc4c4c5ccccc5c5c6ccccc6oc5c43)n2)cc1 ; c1ccc(-c2nc(-n3c4c5ccccc5ccc4c4c5ccccc5c5c6ccccc6oc5c43)nc3ccccc23)cc1]

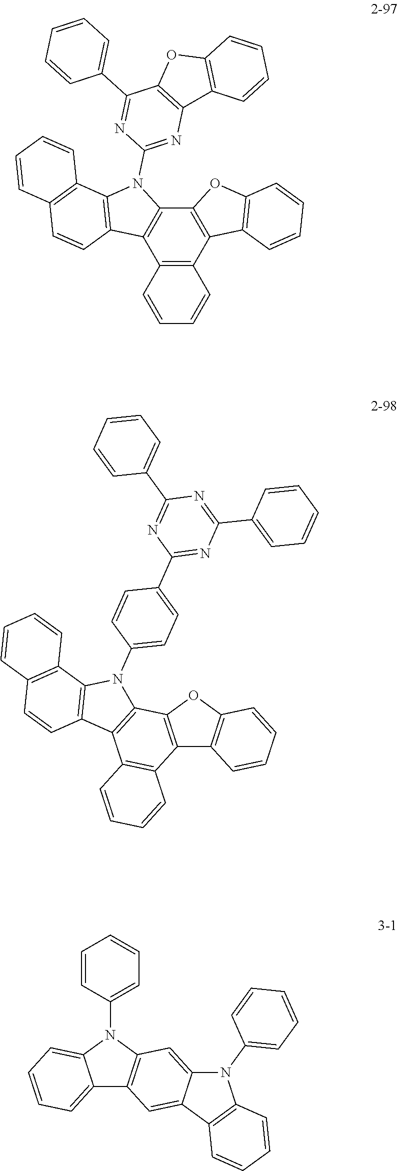 [SMILES: c1ccc(-c2nc(-n3c4c5ccccc5ccc4c4c5ccccc5c5c6ccccc6oc5c43)nc3c2oc2ccccc23)cc1 ; c1ccc(-c2nc(-c3ccccc3)nc(-c3ccc(-n4c5c6ccccc6ccc5c5c6ccccc6c6c7ccccc7oc6c54)cc3)n2)cc1 ; c1ccc(-n2c3ccccc3c3cc4c5ccccc5n(-c5ccccc5)c4cc32)cc1]

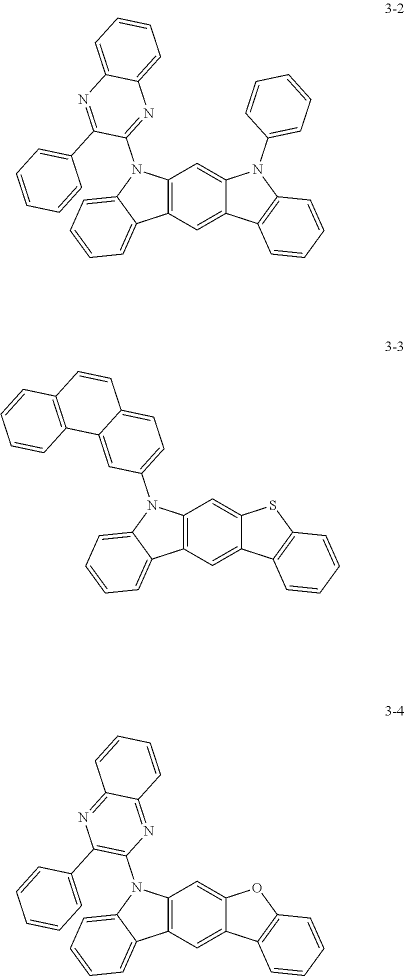 [SMILES: c1ccc(-c2nc3ccccc3nc2-n2c3ccccc3c3cc4c5ccccc5n(-c5ccccc5)c4cc32)cc1 ; c1ccc2c(c1)ccc1ccc(-n3c4ccccc4c4cc5c(cc43)sc3ccccc35)cc12 ; c1ccc(-c2nc3ccccc3nc2-n2c3ccccc3c3cc4c(cc32)oc2ccccc24)cc1]

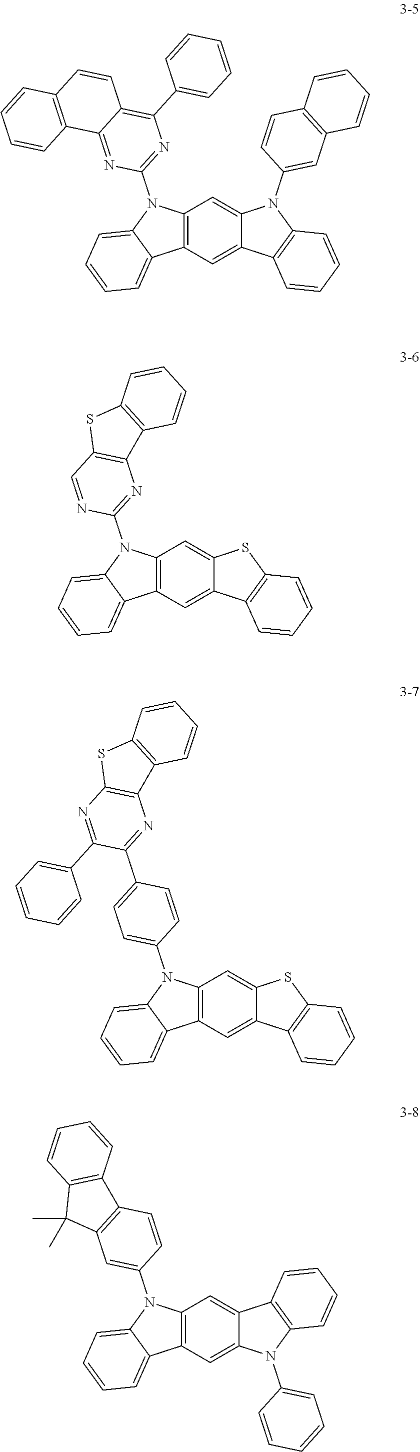 [SMILES: c1ccc(-c2nc(-n3c4ccccc4c4cc5c6ccccc6n(-c6ccc7ccccc7c6)c5cc43)nc3c2ccc2ccccc23)cc1 ; c1ccc2c(c1)sc1cc3c(cc12)c1ccccc1n3-c1ncc2sc3ccccc3c2n1 ; c1ccc(-c2nc3sc4ccccc4c3nc2-c2ccc(-n3c4ccccc4c4cc5c(cc43)sc3ccccc35)cc2)cc1 ; CC1(C)c2ccccc2-c2ccc(-n3c4ccccc4c4cc5c(cc43)c3ccccc3n5-c3ccccc3)cc21]

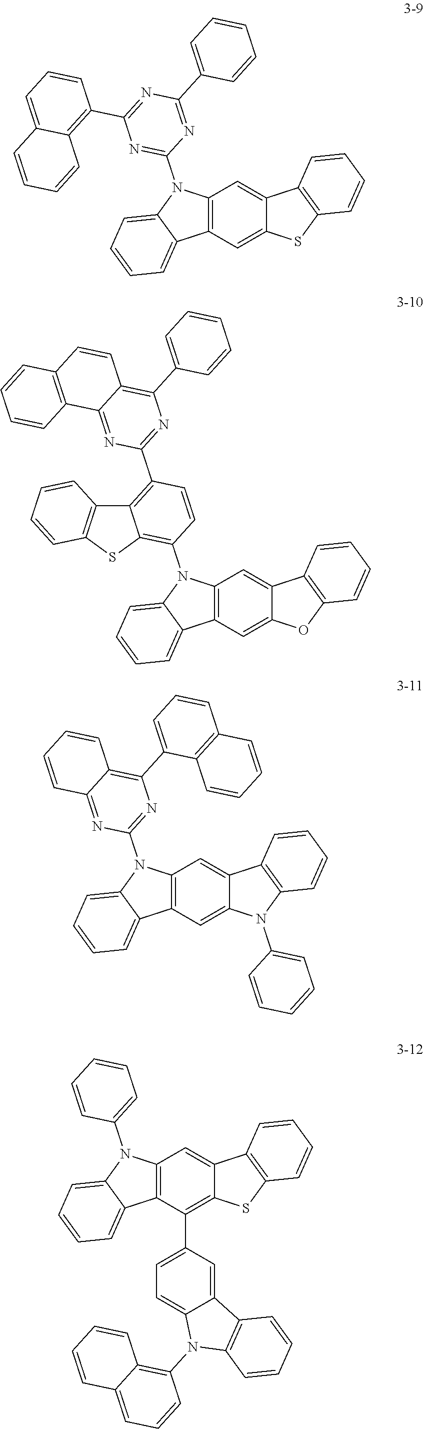

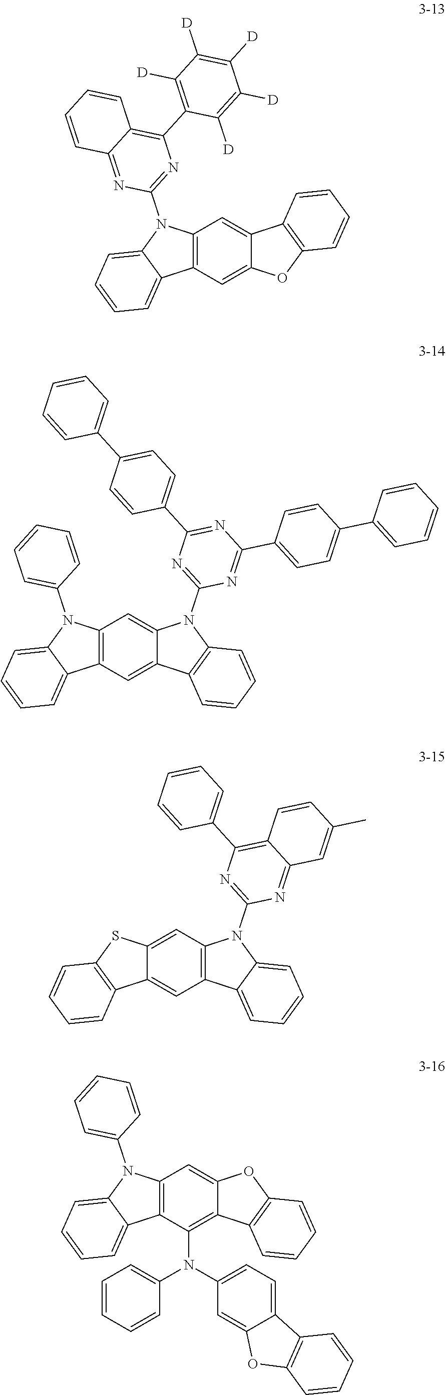 [SMILES: [2H]c1c([2H])c([2H])c(-c2nc(-n3c4ccccc4c4cc5oc6ccccc6c5cc43)nc3ccccc23)c([2H])c1[2H] ; c1ccc(-c2ccc(-c3nc(-c4ccc(-c5ccccc5)cc4)nc(-n4c5ccccc5c5cc6c7ccccc7n(-c7ccccc7)c6cc54)n3)cc2)cc1 ; Cc1ccc2c(-c3ccccc3)nc(-n3c4ccccc4c4cc5c(cc43)sc3ccccc35)nc2c1 ; c1ccc(N(c2ccc3c(c2)oc2ccccc23)c2c3c(cc4c2c2ccccc2n4-c2ccccc2)oc2ccccc23)cc1]

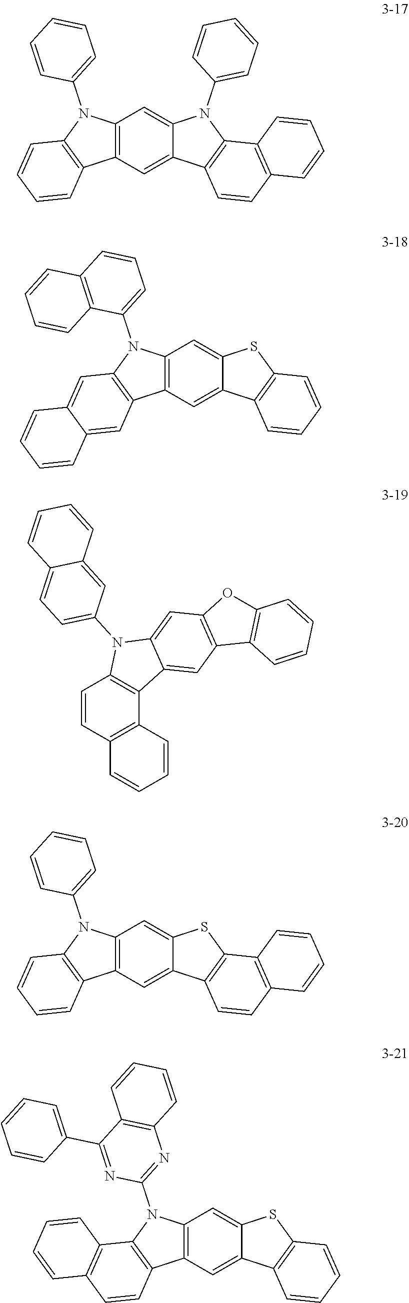

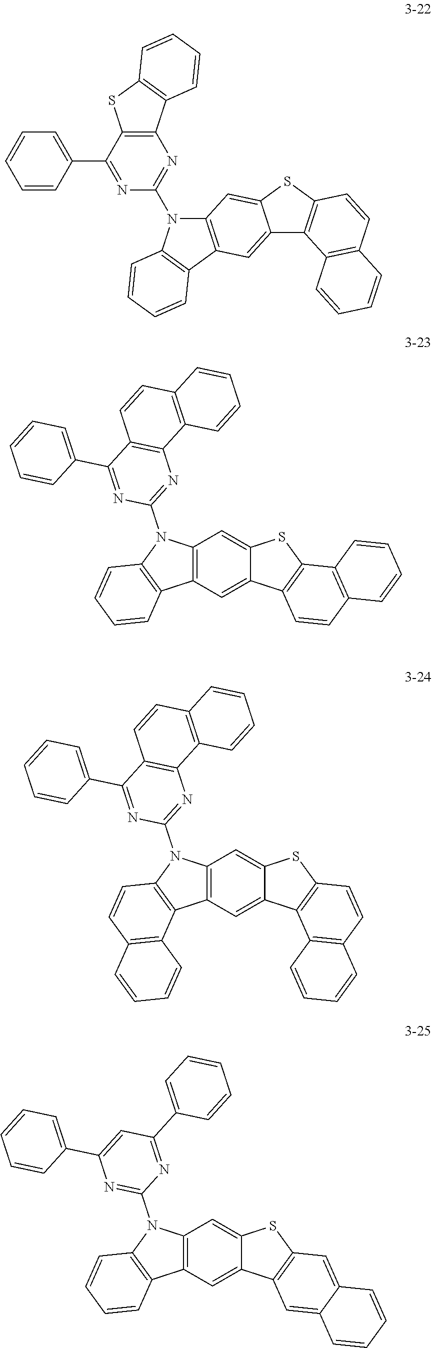 [SMILES: c1ccc(-c2nc(-n3c4ccccc4c4cc5c(cc43)sc3ccc4ccccc4c35)nc3c2sc2ccccc23)cc1 ; c1ccc(-c2nc(-n3c4ccccc4c4cc5c(cc43)sc3c4ccccc4ccc53)nc3c2ccc2ccccc23)cc1 ; c1ccc(-c2nc(-n3c4cc5sc6ccc7ccccc7c6c5cc4c4c5ccccc5ccc43)nc3c2ccc2ccccc23)cc1 ; c1ccc(-c2cc(-c3ccccc3)nc(-n3c4ccccc4c4cc5c(cc43)sc3cc4ccccc4cc35)n2)cc1]

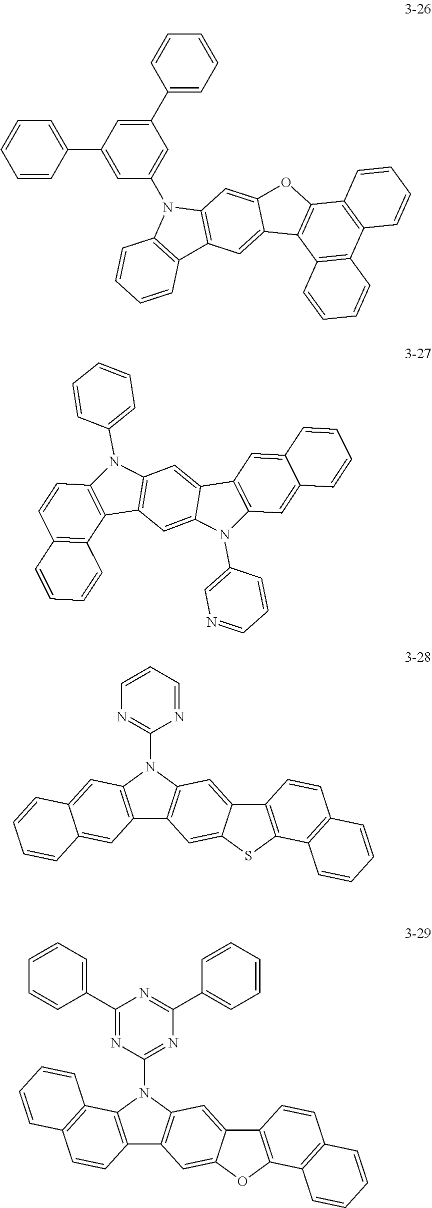 [SMILES: c1ccc(-c2cc(-c3ccccc3)cc(-n3c4ccccc4c4cc5c(cc43)oc3c4ccccc4c4ccccc4c53)c2)cc1 ; c1ccc(-n2c3cc4c5cc6ccccc6cc5n(-c5cccnc5)c4cc3c3c4ccccc4ccc32)cc1 ; c1cnc(-n2c3cc4ccccc4cc3c3cc4sc5c6ccccc6ccc5c4cc32)nc1 ; c1ccc(-c2nc(-c3ccccc3)nc(-n3c4cc5c(cc4c4ccc6ccccc6c43)oc3c4ccccc4ccc53)n2)cc1]

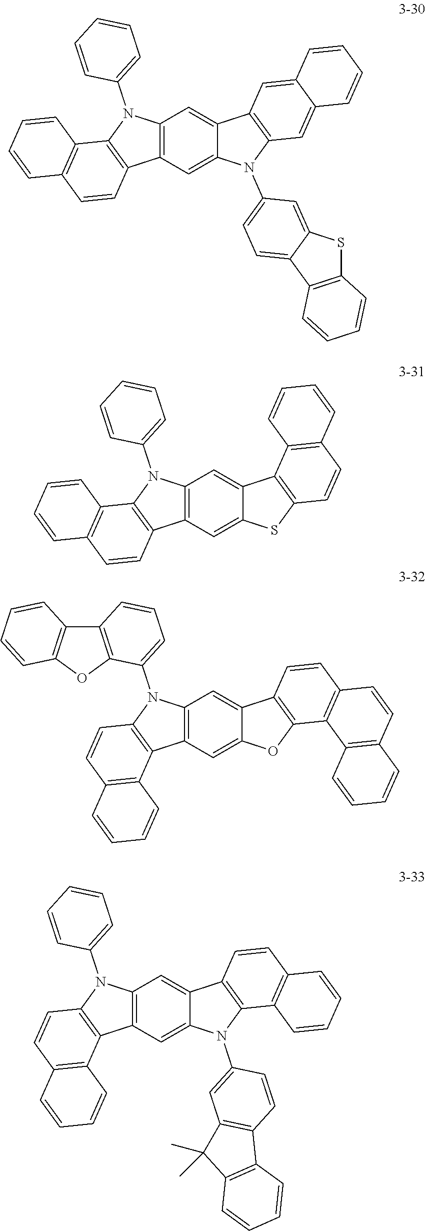

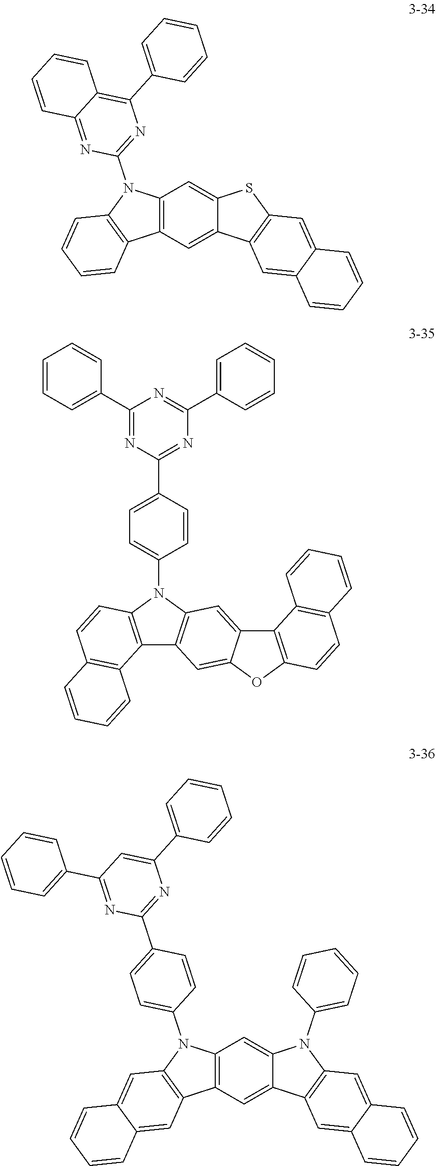 [SMILES: c1ccc(-c2nc(-n3c4ccccc4c4cc5c(cc43)sc3cc4ccccc4cc35)nc3ccccc23)cc1 ; c1ccc(-c2nc(-c3ccccc3)nc(-c3ccc(-n4c5cc6c(cc5c5c7ccccc7ccc54)oc4ccc5ccccc5c46)cc3)n2)cc1 ; c1ccc(-c2cc(-c3ccccc3)nc(-c3ccc(-n4c5cc6ccccc6cc5c5cc6c7cc8ccccc8cc7n(-c7ccccc7)c6cc54)cc3)n2)cc1]

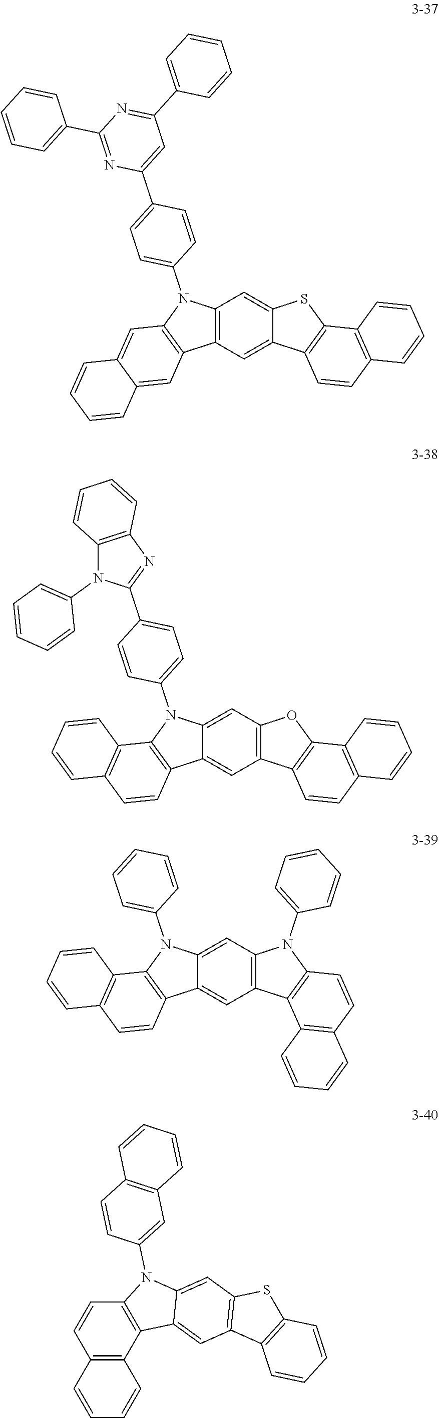 [SMILES: c1ccc(-c2cc(-c3ccc(-n4c5cc6ccccc6cc5c5cc6c(cc54)sc4c5ccccc5ccc64)cc3)nc(-c3ccccc3)n2)cc1 ; c1ccc(-n2c(-c3ccc(-n4c5cc6oc7c8ccccc8ccc7c6cc5c5ccc6ccccc6c54)cc3)nc3ccccc32)cc1 ; c1ccc(-n2c3cc4c(cc3c3c5ccccc5ccc32)c2ccc3ccccc3c2n4-c2ccccc2)cc1 ; c1ccc2cc(-n3c4cc5sc6ccccc6c5cc4c4c5ccccc5ccc43)ccc2c1]

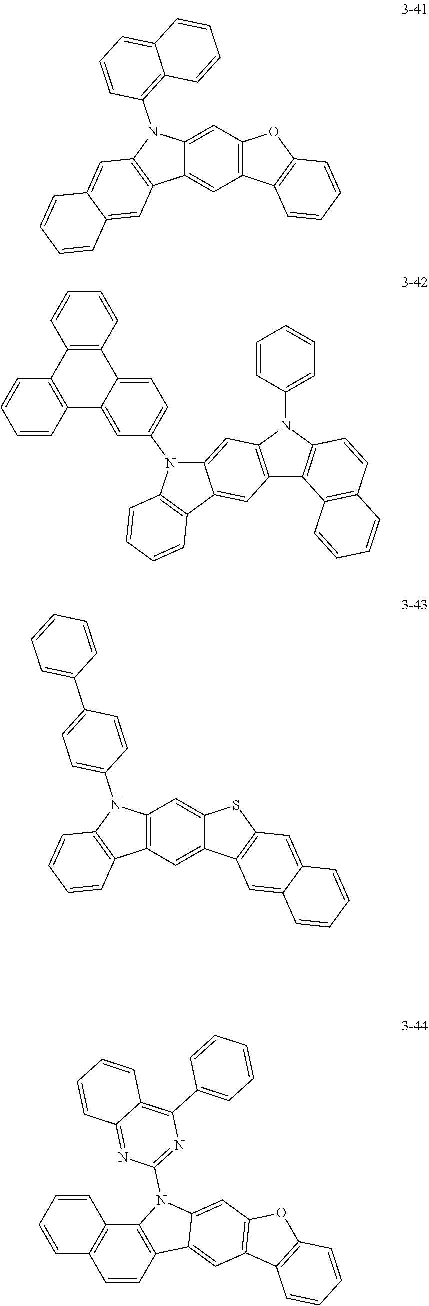

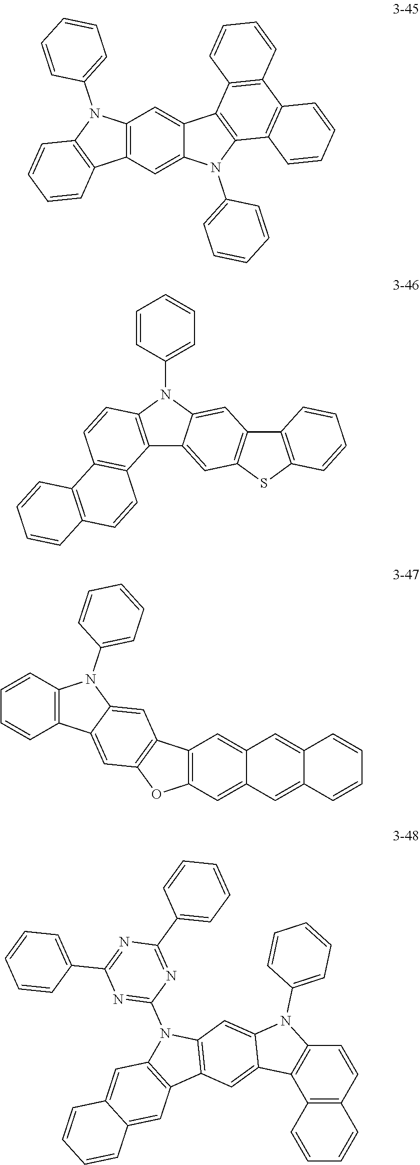 [SMILES: c1ccc(-n2c3ccccc3c3cc4c(cc32)c2c3ccccc3c3ccccc3c2n4-c2ccccc2)cc1 ; c1ccc(-n2c3cc4c(cc3c3c5ccc6ccccc6c5ccc32)sc2ccccc24)cc1 ; c1ccc(-n2c3ccccc3c3cc4oc5cc6cc7ccccc7cc6cc5c4cc32)cc1 ; c1ccc(-c2nc(-c3ccccc3)nc(-n3c4cc5ccccc5cc4c4cc5c6c7ccccc7ccc6n(-c6ccccc6)c5cc43)n2)cc1]

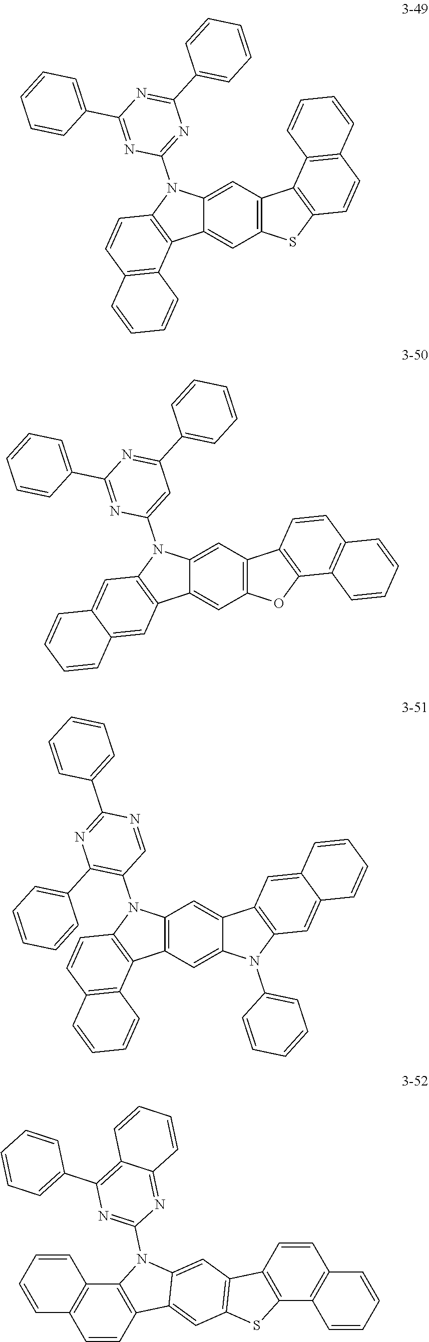

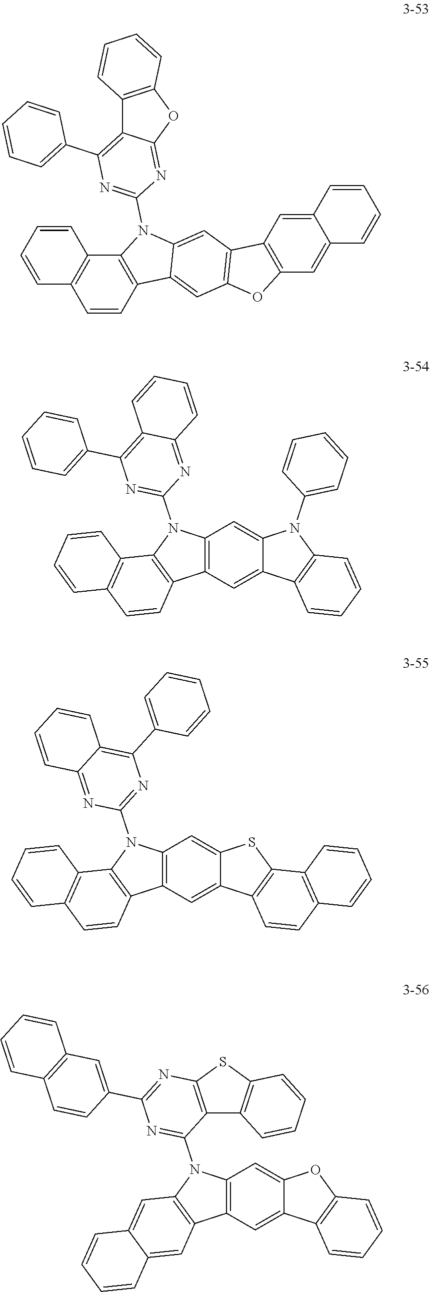 [SMILES: c1ccc(-c2nc(-n3c4cc5c(cc4c4ccc6ccccc6c43)oc3cc4ccccc4cc35)nc3oc4ccccc4c23)cc1 ; c1ccc(-c2nc(-n3c4cc5c(cc4c4ccc6ccccc6c43)c3ccccc3n5-c3ccccc3)nc3ccccc23)cc1 ; c1ccc(-c2nc(-n3c4cc5sc6c7ccccc7ccc6c5cc4c4ccc5ccccc5c43)nc3ccccc23)cc1 ; c1ccc2cc(-c3nc(-n4c5cc6ccccc6cc5c5cc6c(cc54)oc4ccccc46)c4c(n3)sc3ccccc34)ccc2c1]

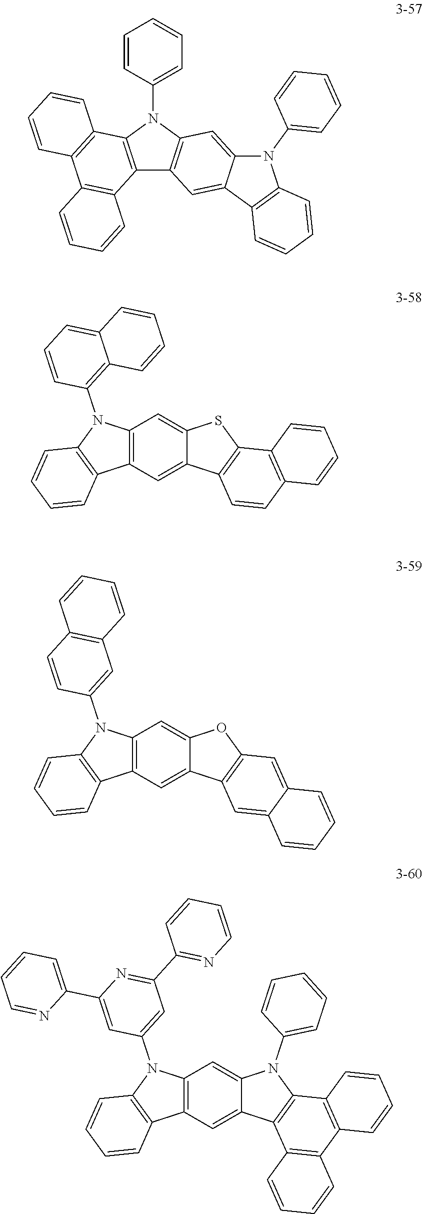 [SMILES: c1ccc(-n2c3ccccc3c3cc4c5c6ccccc6c6ccccc6c5n(-c5ccccc5)c4cc32)cc1 ; c1ccc2c(-n3c4ccccc4c4cc5c(cc43)sc3c4ccccc4ccc53)cccc2c1 ; c1ccc2cc(-n3c4ccccc4c4cc5c(cc43)oc3cc4ccccc4cc35)ccc2c1 ; c1ccc(-n2c3cc4c(cc3c3c5ccccc5c5ccccc5c32)c2ccccc2n4-c2cc(-c3ccccn3)nc(-c3ccccn3)c2)cc1]

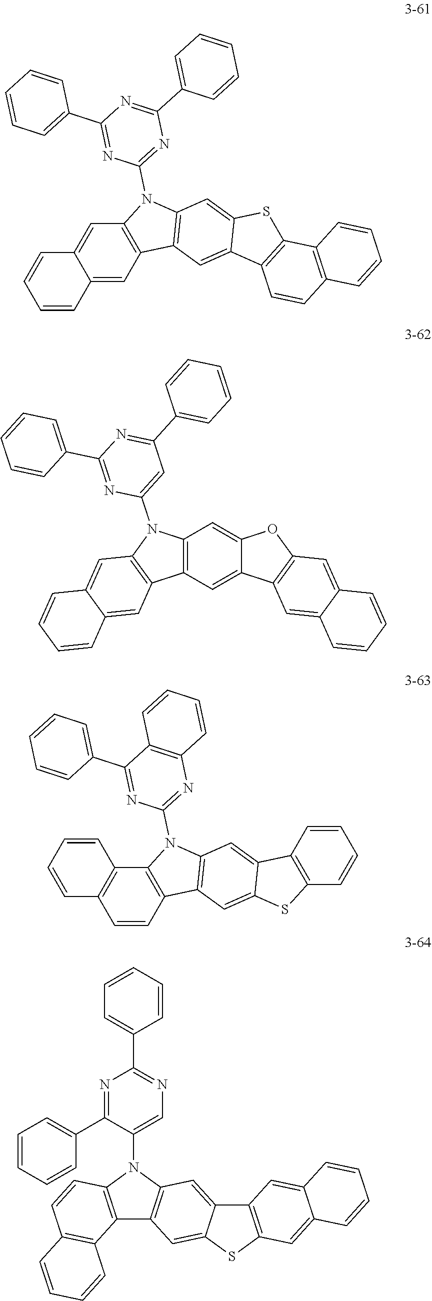

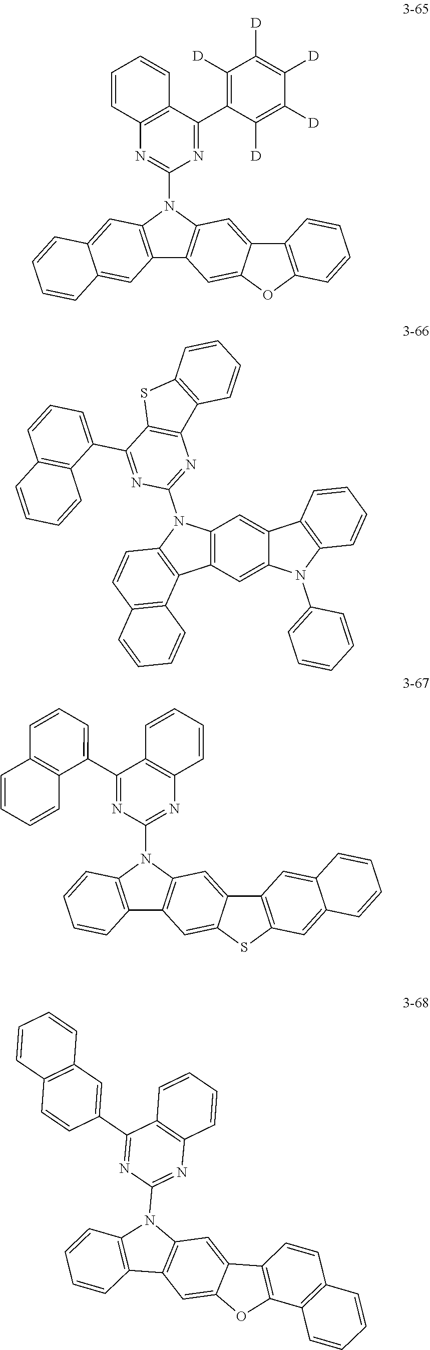

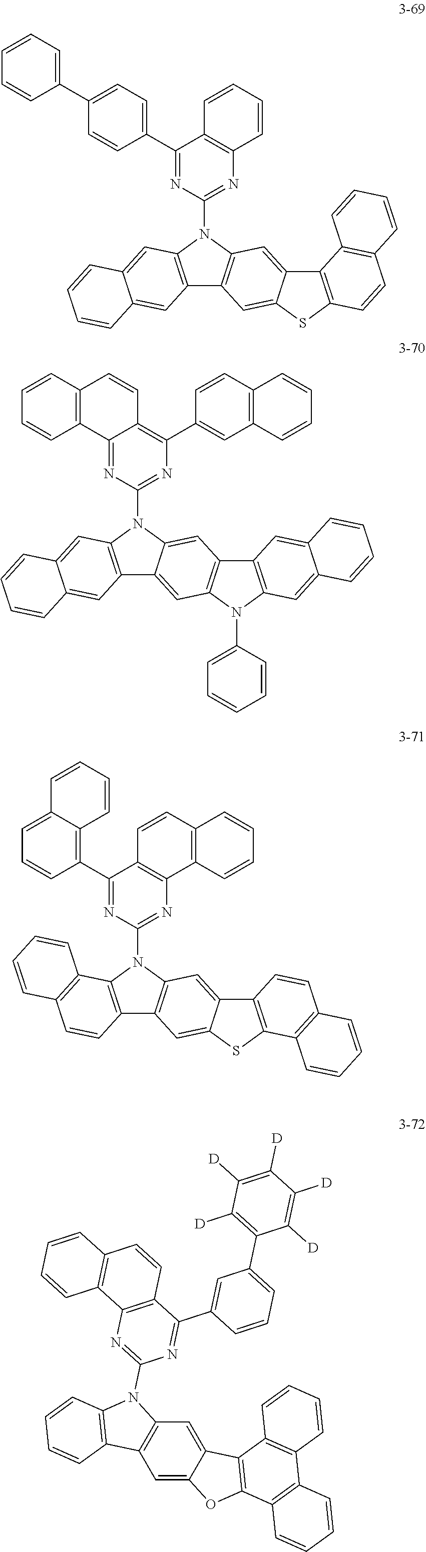

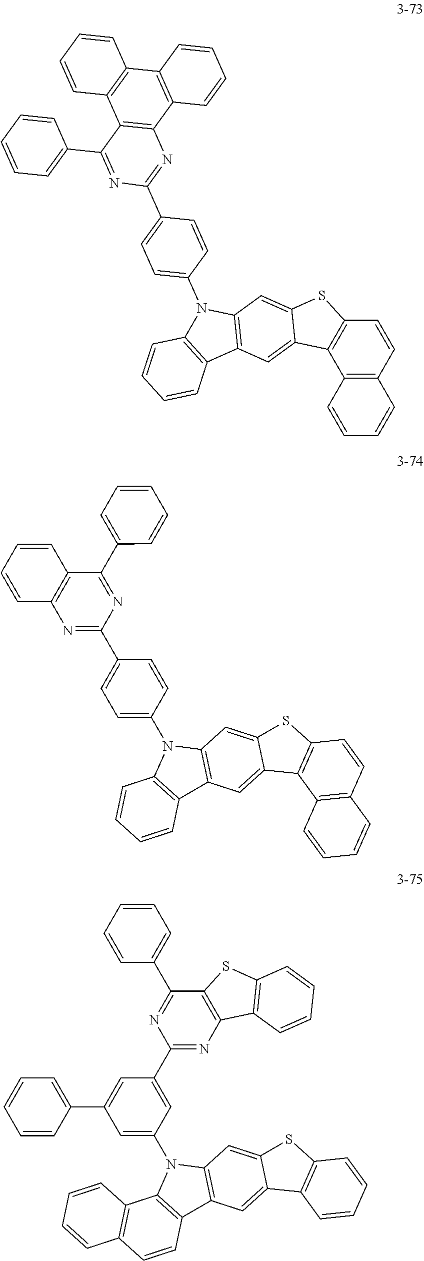 [SMILES: c1ccc(-c2nc(-c3ccc(-n4c5ccccc5c5cc6c(cc54)sc4ccc5ccccc5c46)cc3)nc3c4ccccc4c4ccccc4c23)cc1 ; c1ccc(-c2nc(-c3ccc(-n4c5ccccc5c5cc6c(cc54)sc4ccc5ccccc5c46)cc3)nc3ccccc23)cc1 ; c1ccc(-c2cc(-c3nc(-c4ccccc4)c4sc5ccccc5c4n3)cc(-n3c4cc5sc6ccccc6c5cc4c4ccc5ccccc5c43)c2)cc1]

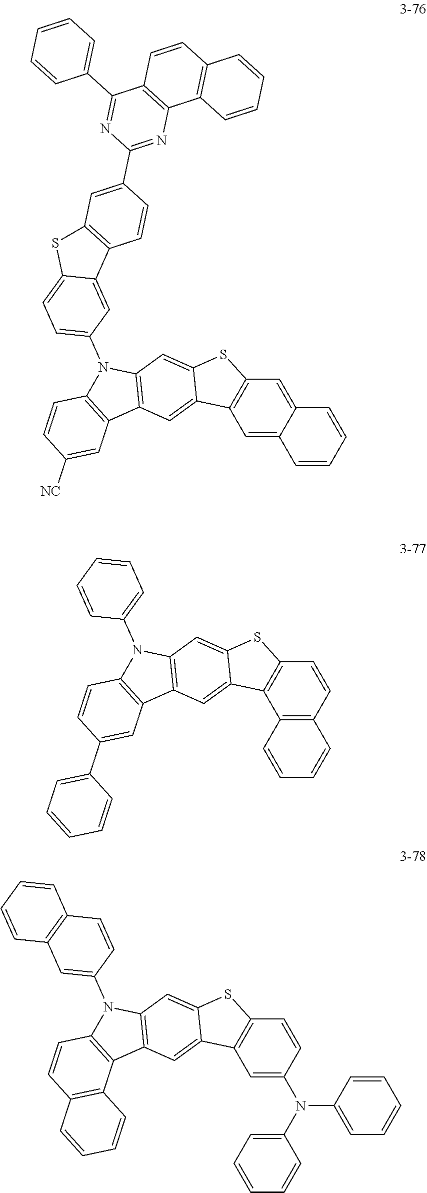 [SMILES: N#Cc1ccc2c(c1)c1cc3c(cc1n2-c1ccc2sc4cc(-c5nc(-c6ccccc6)c6ccc7ccccc7c6n5)ccc4c2c1)sc1cc2ccccc2cc13 ; c1ccc(-c2ccc3c(c2)c2cc4c(cc2n3-c2ccccc2)sc2ccc3ccccc3c24)cc1 ; c1ccc(N(c2ccccc2)c2ccc3sc4cc5c(cc4c3c2)c2c3ccccc3ccc2n5-c2ccc3ccccc3c2)cc1]

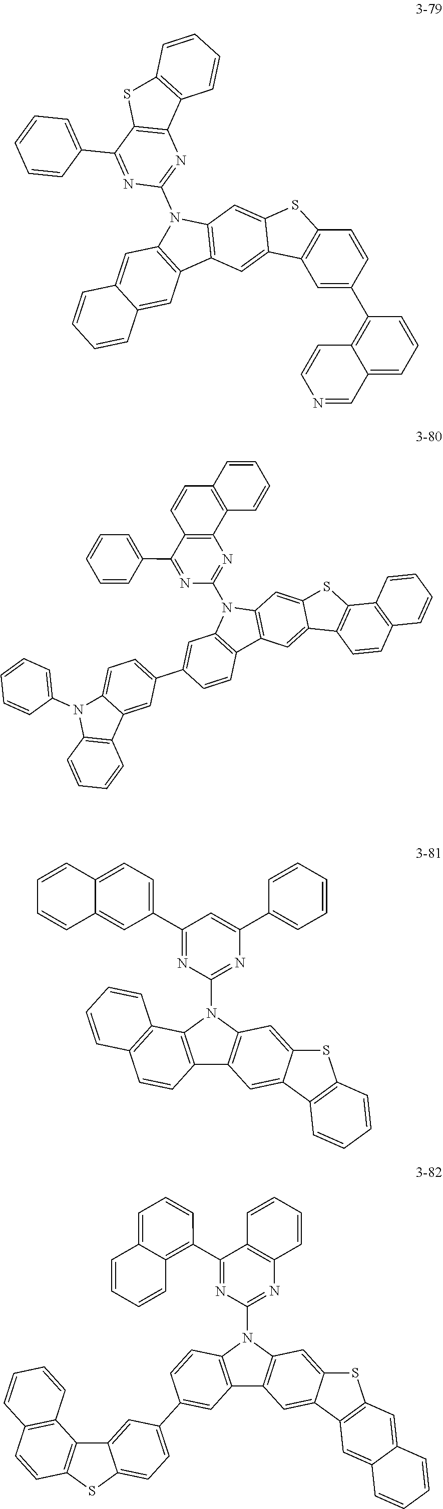 [SMILES: c1ccc(-c2nc(-n3c4cc5ccccc5cc4c4cc5c(cc43)sc3ccc(-c4cccc6cnccc46)cc35)nc3c2sc2ccccc23)cc1 ; c1ccc(-c2nc(-n3c4cc(-c5ccc6c(c5)c5ccccc5n6-c5ccccc5)ccc4c4cc5c(cc43)sc3c4ccccc4ccc53)nc3c2ccc2ccccc23)cc1 ; c1ccc(-c2cc(-c3ccc4ccccc4c3)nc(-n3c4cc5sc6ccccc6c5cc4c4ccc5ccccc5c43)n2)cc1 ; c1ccc2cc3c(cc2c1)sc1cc2c(cc13)c1cc(-c3ccc4sc5ccc6ccccc6c5c4c3)ccc1n2-c1nc(-c2cccc3ccccc23)c2ccccc2n1]

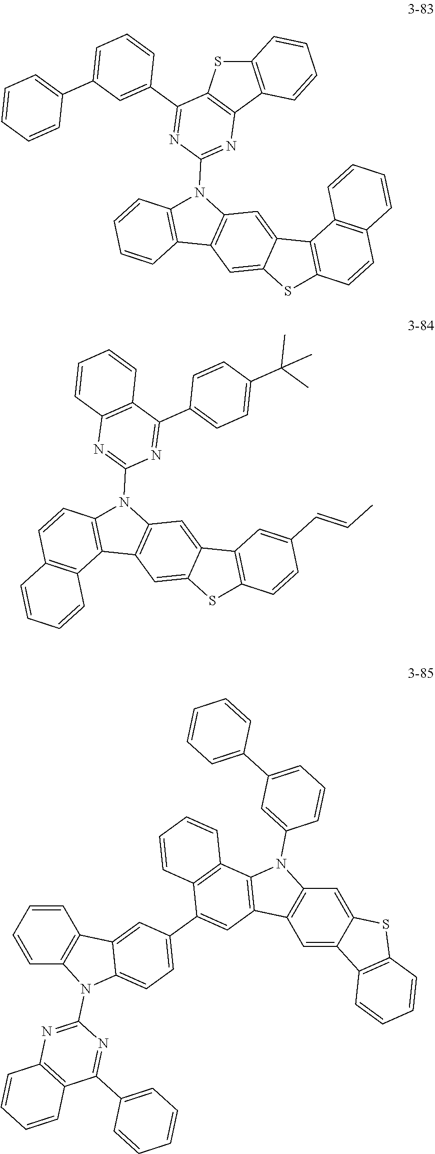

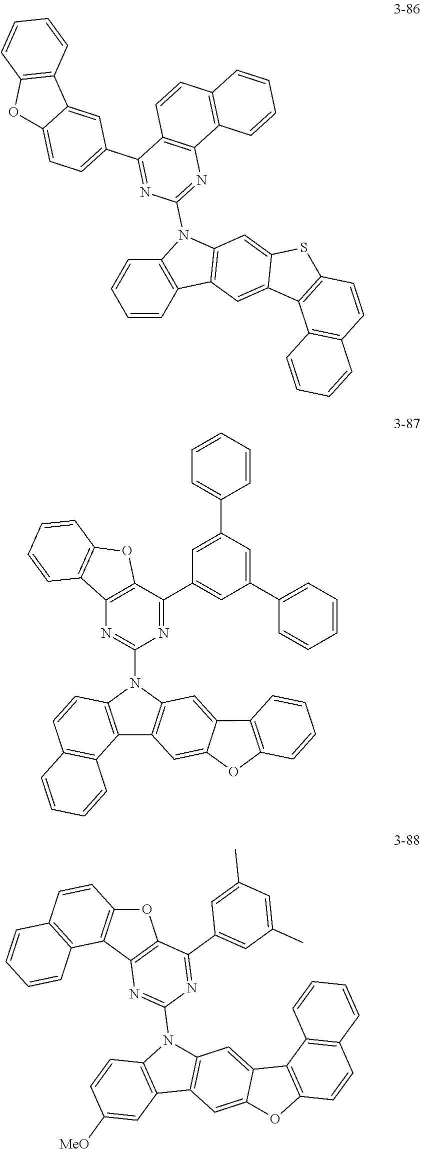 [SMILES: c1ccc2c(c1)ccc1c(-c3ccc4oc5ccccc5c4c3)nc(-n3c4ccccc4c4cc5c(cc43)sc3ccc4ccccc4c35)nc12 ; c1ccc(-c2cc(-c3ccccc3)cc(-c3nc(-n4c5cc6c(cc5c5c7ccccc7ccc54)oc4ccccc46)nc4c3oc3ccccc34)c2)cc1 ; COc1ccc2c(c1)c1cc3oc4ccc5ccccc5c4c3cc1n2-c1nc(-c2cc(C)cc(C)c2)c2oc3ccc4ccccc4c3c2n1]

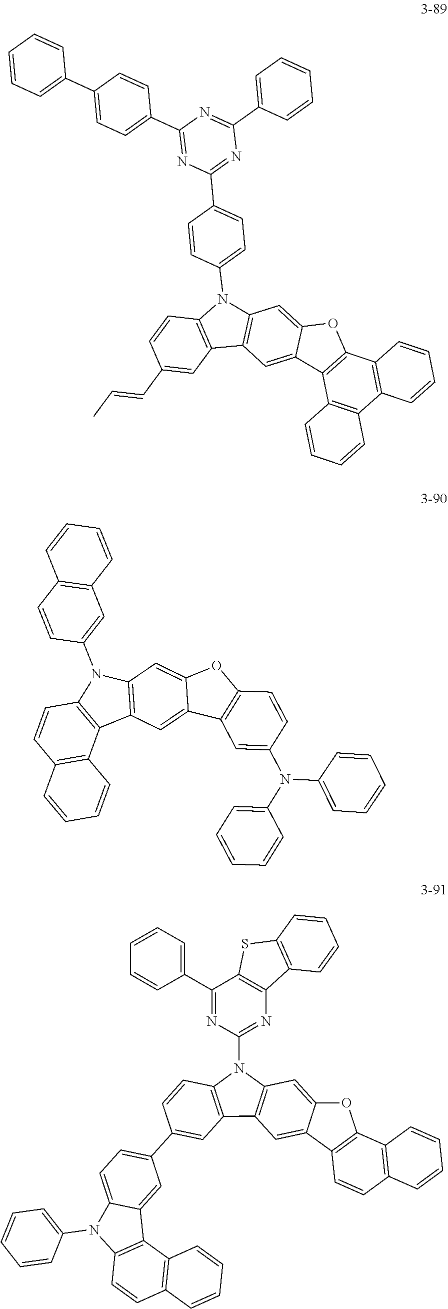 [SMILES: C/C=C/c1ccc2c(c1)c1cc3c(cc1n2-c1ccc(-c2nc(-c4ccccc4)nc(-c4ccc(-c5ccccc5)cc4)n2)cc1)oc1c2ccccc2c2ccccc2c31 ; c1ccc(N(c2ccccc2)c2ccc3oc4cc5c(cc4c3c2)c2c3ccccc3ccc2n5-c2ccc3ccccc3c2)cc1 ; c1ccc(-c2nc(-n3c4ccc(-c5ccc6c(c5)c5c7ccccc7ccc5n6-c5ccccc5)cc4c4cc5c(cc43)oc3c4ccccc4ccc53)nc3c2sc2ccccc23)cc1]

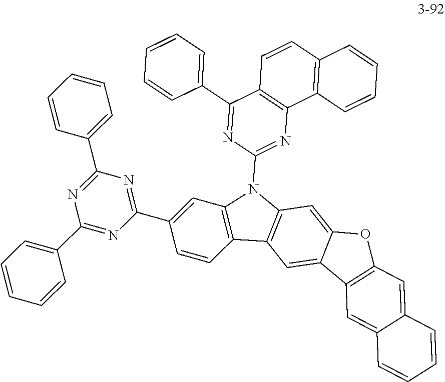 [SMILES: c1ccc(-c2nc(-c3ccccc3)nc(-c3ccc4c5cc6c(cc5n(-c5nc(-c7ccccc7)c7ccc8ccccc8c7n5)c4c3)oc3cc4ccccc4cc36)n2)cc1]

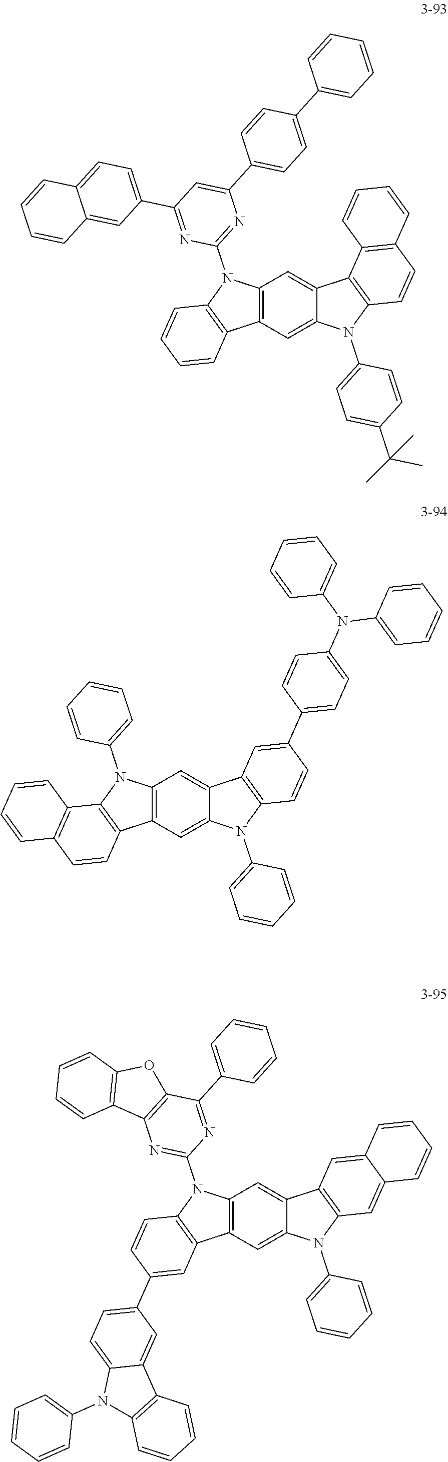 [SMILES: CC(C)(C)c1ccc(-n2c3cc4c5ccccc5n(-c5nc(-c6ccc(-c7ccccc7)cc6)cc(-c6ccc7ccccc7c6)n5)c4cc3c3c4ccccc4ccc32)cc1 ; c1ccc(N(c2ccccc2)c2ccc(-c3ccc4c(c3)c3cc5c(cc3n4-c3ccccc3)c3ccc4ccccc4c3n5-c3ccccc3)cc2)cc1 ; c1ccc(-c2nc(-n3c4ccc(-c5ccc6c(c5)c5ccccc5n6-c5ccccc5)cc4c4cc5c(cc43)c3cc4ccccc4cc3n5-c3ccccc3)nc3c2oc2ccccc23)cc1]

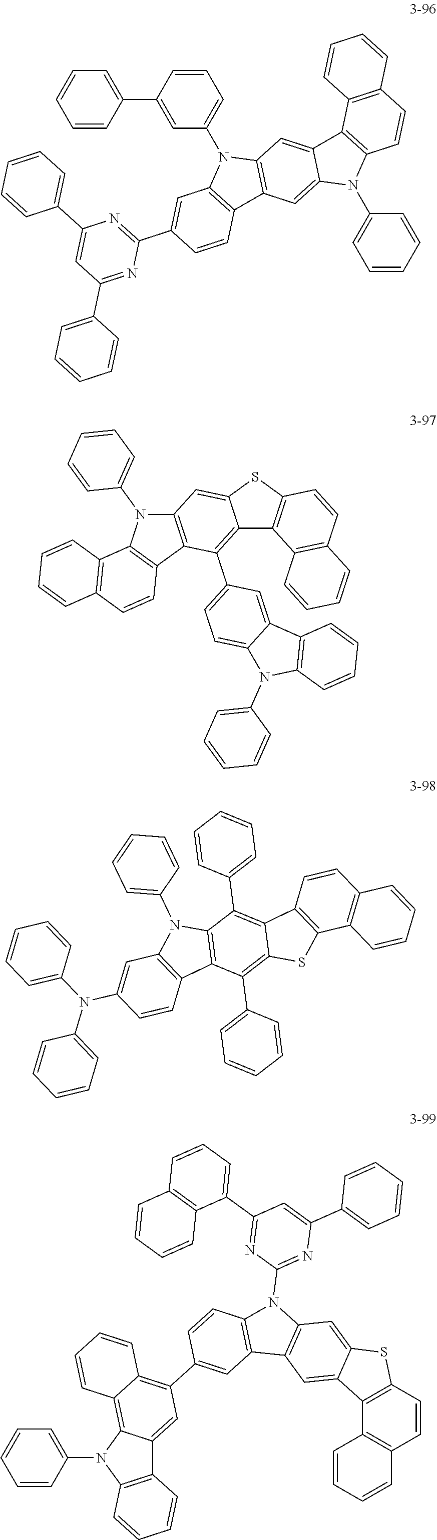

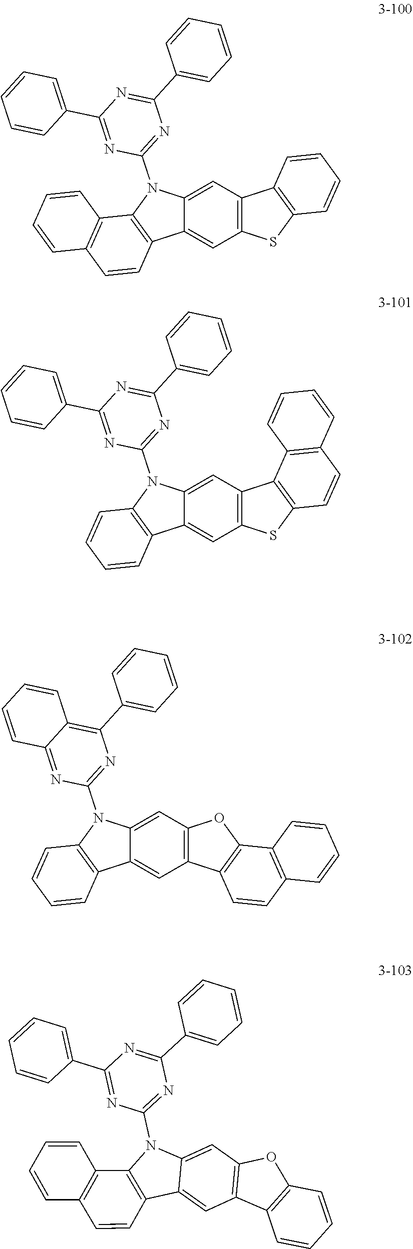 [SMILES: c1ccc(-c2nc(-c3ccccc3)nc(-n3c4cc5c(cc4c4ccc6ccccc6c43)sc3ccccc35)n2)cc1 ; c1ccc(-c2nc(-c3ccccc3)nc(-n3c4ccccc4c4cc5sc6ccc7ccccc7c6c5cc43)n2)cc1 ; c1ccc(-c2nc(-n3c4ccccc4c4cc5c(cc43)oc3c4ccccc4ccc53)nc3ccccc23)cc1 ; c1ccc(-c2nc(-c3ccccc3)nc(-n3c4cc5oc6ccccc6c5cc4c4ccc5ccccc5c43)n2)cc1]

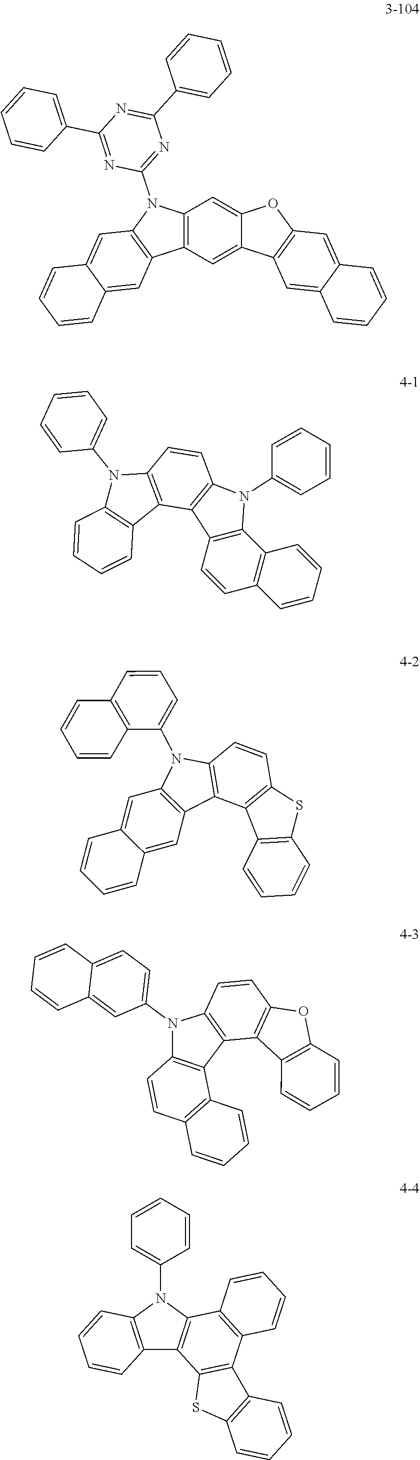 [SMILES: c1ccc(-c2nc(-c3ccccc3)nc(-n3c4cc5ccccc5cc4c4cc5c(cc43)oc3cc4ccccc4cc35)n2)cc1 ; c1ccc(-n2c3ccccc3c3c4c5ccc6ccccc6c5n(-c5ccccc5)c4ccc32)cc1 ; c1ccc2cc3c(cc2c1)c1c2c(ccc1n3-c1cccc3ccccc13)sc1ccccc12 ; c1ccc2cc(-n3c4ccc5ccccc5c4c4c5c(ccc43)oc3ccccc35)ccc2c1 ; c1ccc(-n2c3ccccc3c3c4sc5ccccc5c4c4ccccc4c32)cc1]

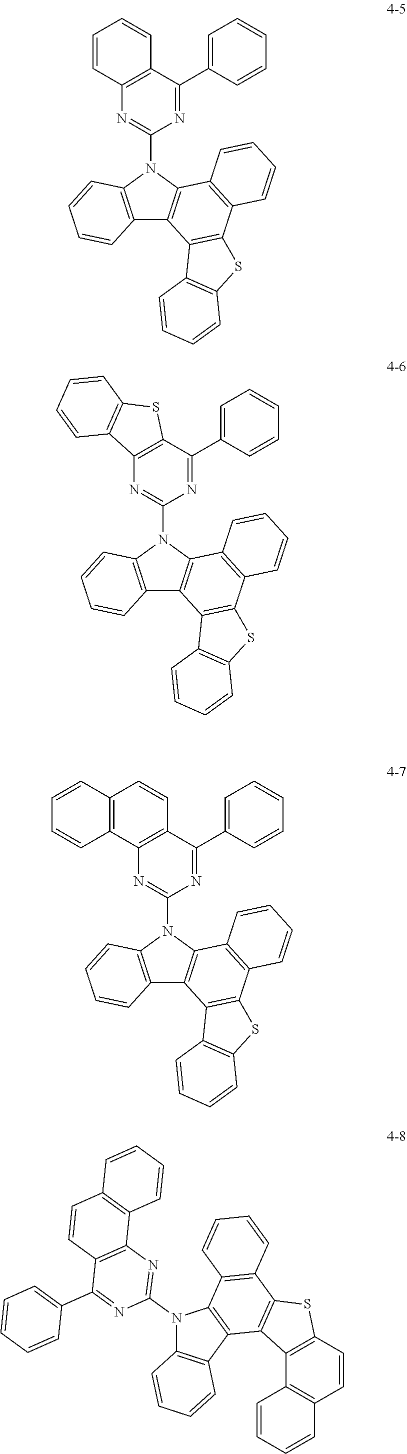 [SMILES: c1ccc(-c2nc(-n3c4ccccc4c4c5c6ccccc6sc5c5ccccc5c43)nc3ccccc23)cc1 ; c1ccc(-c2nc(-n3c4ccccc4c4c5c6ccccc6sc5c5ccccc5c43)nc3c2sc2ccccc23)cc1 ; c1ccc(-c2nc(-n3c4ccccc4c4c5c6ccccc6sc5c5ccccc5c43)nc3c2ccc2ccccc23)cc1 ; c1ccc(-c2nc(-n3c4ccccc4c4c5c(sc6ccc7ccccc7c65)c5ccccc5c43)nc3c2ccc2ccccc23)cc1]

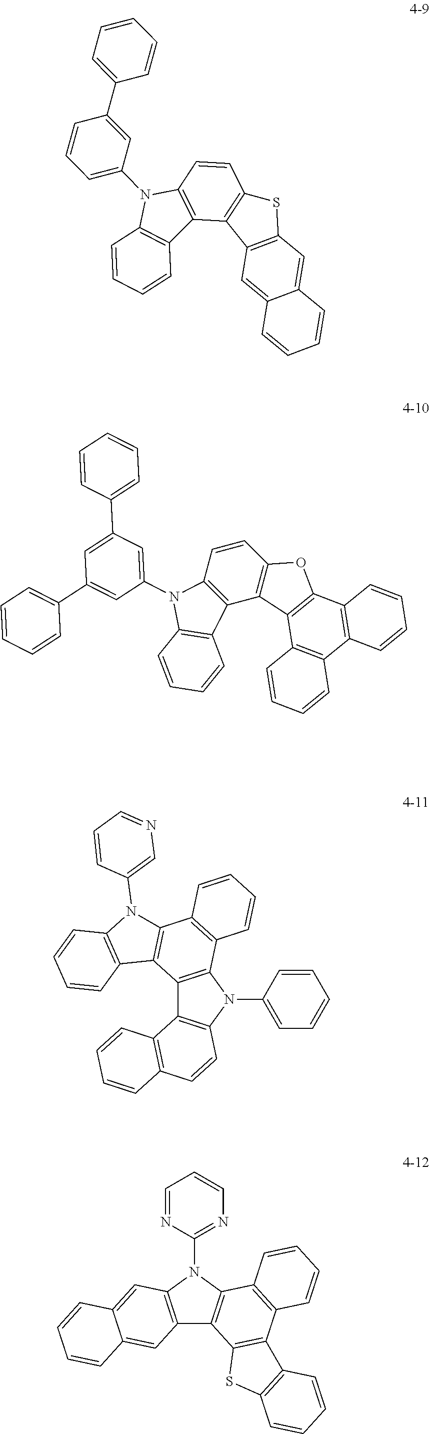 [SMILES: c1ccc(-c2cccc(-n3c4ccccc4c4c5c(ccc43)sc3cc4ccccc4cc35)c2)cc1 ; c1ccc(-c2cc(-c3ccccc3)cc(-n3c4ccccc4c4c5c(ccc43)oc3c4ccccc4c4ccccc4c35)c2)cc1 ; c1ccc(-n2c3ccc4ccccc4c3c3c4c5ccccc5n(-c5cccnc5)c4c4ccccc4c32)cc1 ; c1cnc(-n2c3cc4ccccc4cc3c3c4sc5ccccc5c4c4ccccc4c32)nc1]

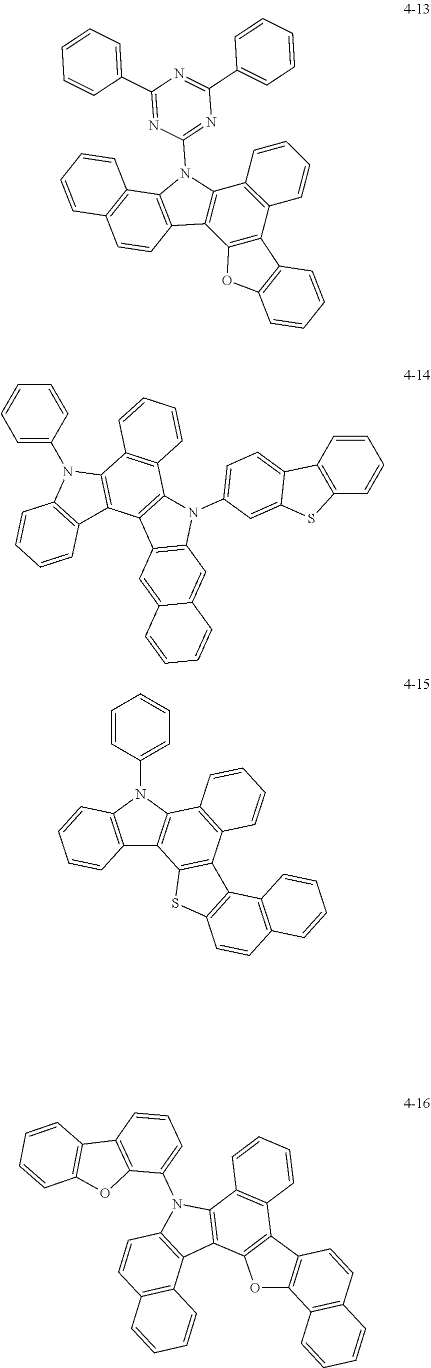

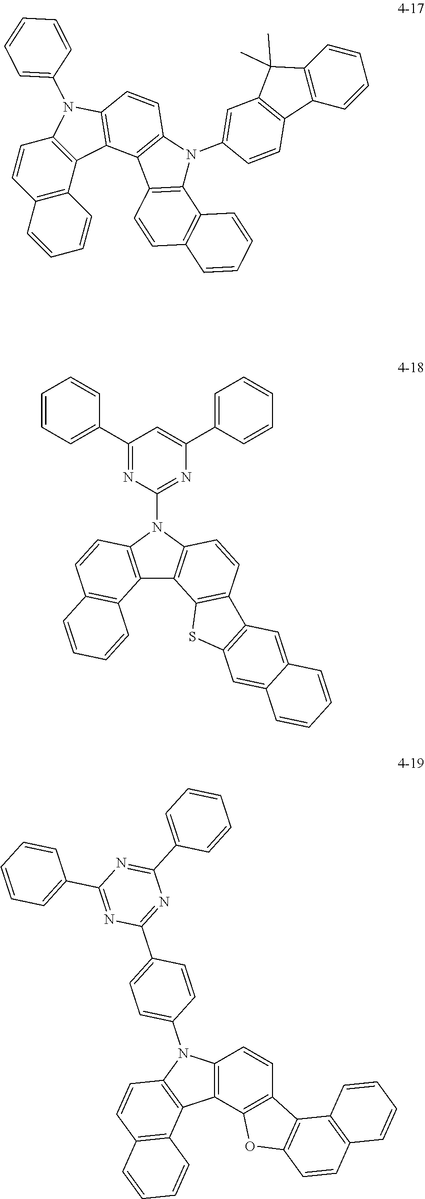 [SMILES: CC1(C)c2ccccc2-c2ccc(-n3c4ccc5c(c6c7ccccc7ccc6n5-c5ccccc5)c4c4ccc5ccccc5c43)cc21 ; c1ccc(-c2cc(-c3ccccc3)nc(-n3c4ccc5ccccc5c4c4c5sc6cc7ccccc7cc6c5ccc43)n2)cc1 ; c1ccc(-c2nc(-c3ccccc3)nc(-c3ccc(-n4c5ccc6ccccc6c5c5c6oc7ccc8ccccc8c7c6ccc54)cc3)n2)cc1]

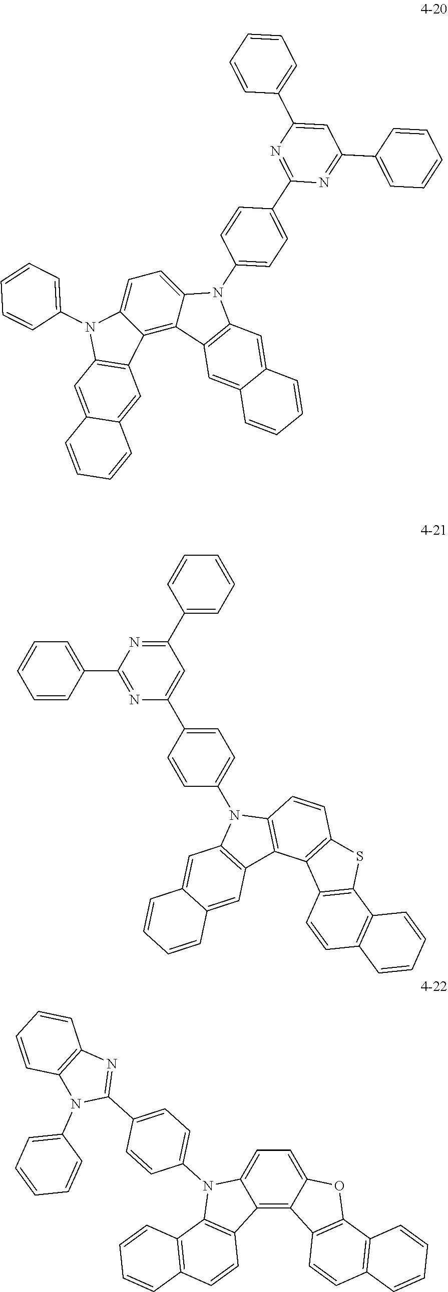 [SMILES: c1ccc(-c2cc(-c3ccccc3)nc(-c3ccc(-n4c5cc6ccccc6cc5c5c6c7cc8ccccc8cc7n(-c7ccccc7)c6ccc54)cc3)n2)cc1 ; c1ccc(-c2cc(-c3ccc(-n4c5cc6ccccc6cc5c5c6c(ccc54)sc4c5ccccc5ccc46)cc3)nc(-c3ccccc3)n2)cc1 ; c1ccc(-n2c(-c3ccc(-n4c5ccc6oc7c8ccccc8ccc7c6c5c5ccc6ccccc6c54)cc3)nc3ccccc32)cc1]

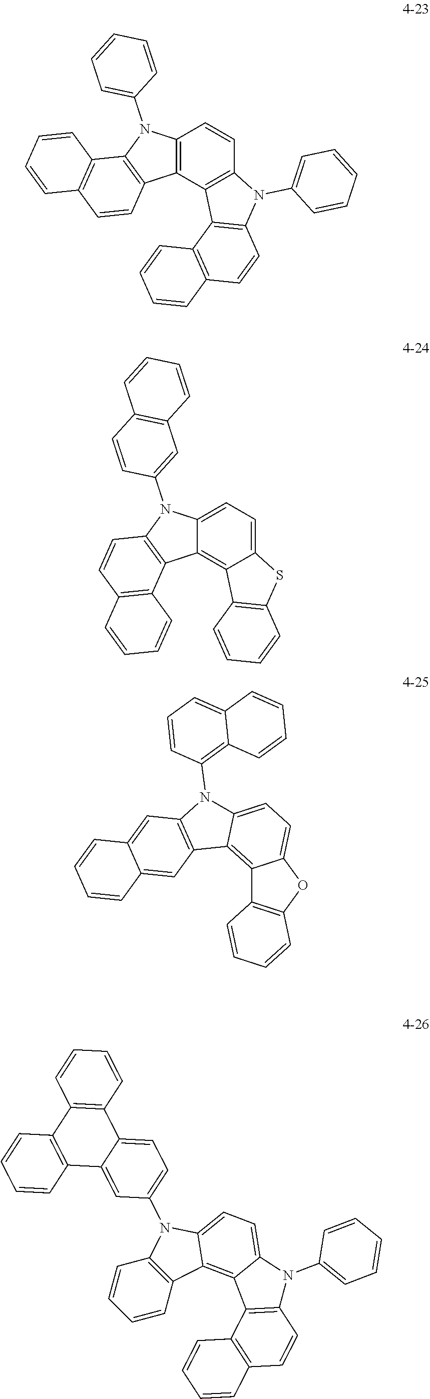

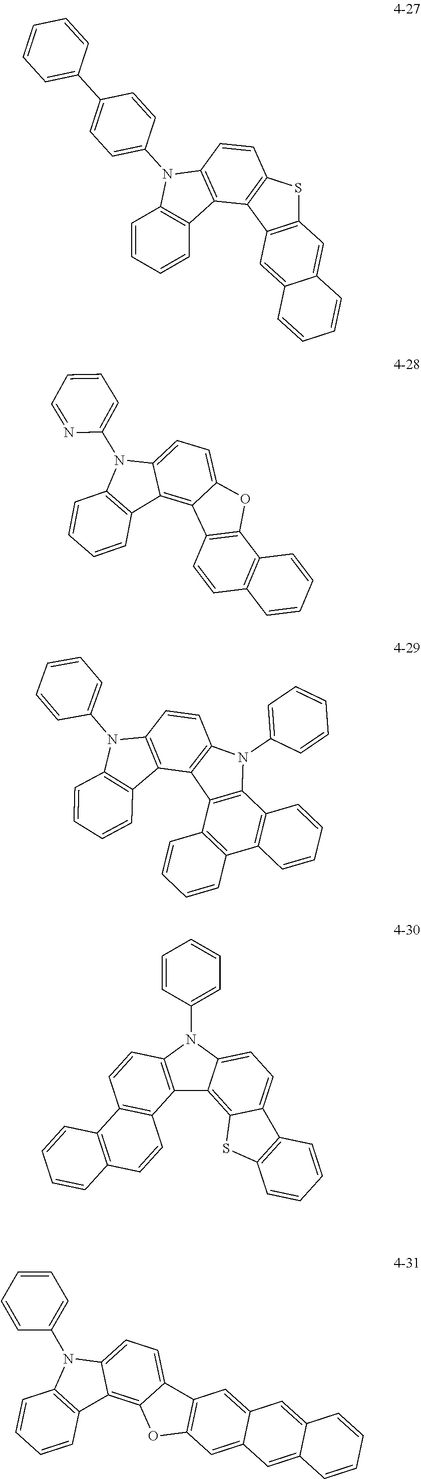

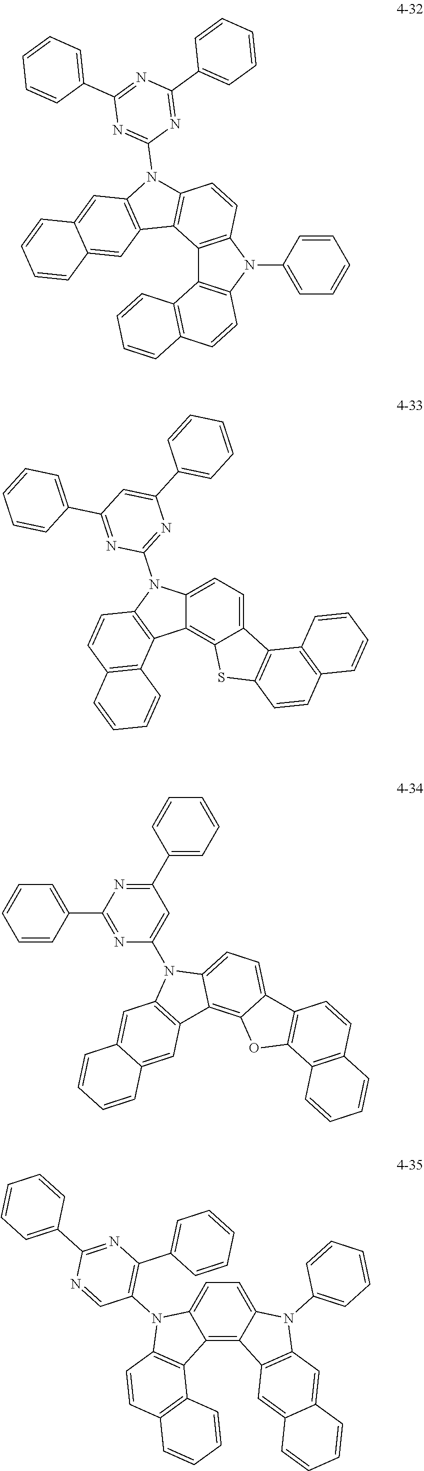 [SMILES: c1ccc(-c2nc(-c3ccccc3)nc(-n3c4cc5ccccc5cc4c4c5c6c7ccccc7ccc6n(-c6ccccc6)c5ccc43)n2)cc1 ; c1ccc(-c2cc(-c3ccccc3)nc(-n3c4ccc5ccccc5c4c4c5sc6ccc7ccccc7c6c5ccc43)n2)cc1 ; c1ccc(-c2cc(-n3c4cc5ccccc5cc4c4c5oc6c7ccccc7ccc6c5ccc43)nc(-c3ccccc3)n2)cc1 ; c1ccc(-c2ncc(-n3c4ccc5ccccc5c4c4c5c6cc7ccccc7cc6n(-c6ccccc6)c5ccc43)c(-c3ccccc3)n2)cc1]

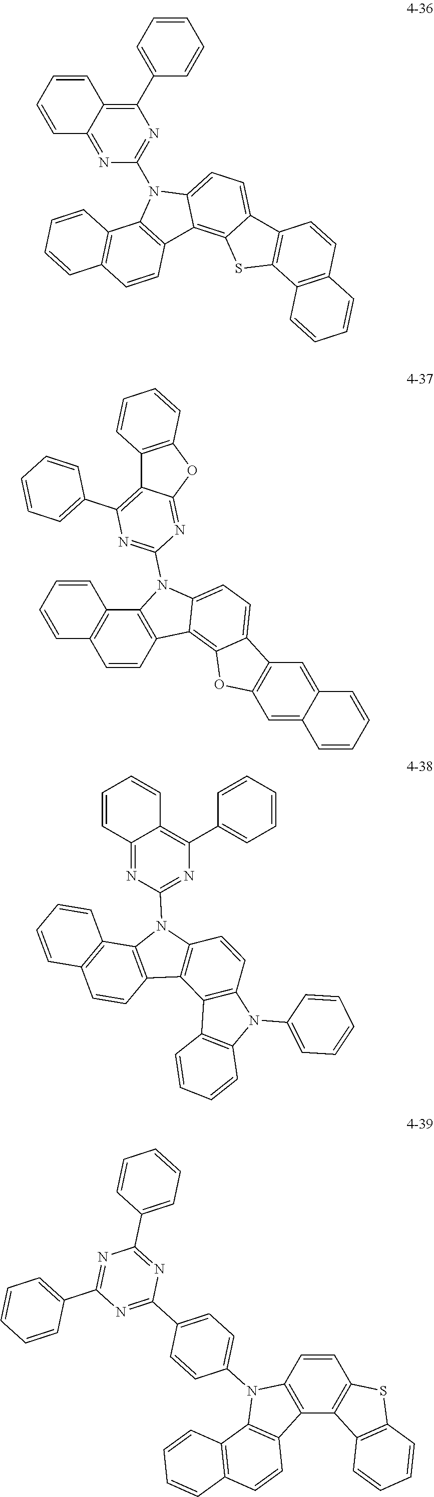 [SMILES: c1ccc(-c2nc(-n3c4ccc5c6ccc7ccccc7c6sc5c4c4ccc5ccccc5c43)nc3ccccc23)cc1 ; c1ccc(-c2nc(-n3c4ccc5c6cc7ccccc7cc6oc5c4c4ccc5ccccc5c43)nc3oc4ccccc4c23)cc1 ; c1ccc(-c2nc(-n3c4ccc5c(c6ccccc6n5-c5ccccc5)c4c4ccc5ccccc5c43)nc3ccccc23)cc1 ; c1ccc(-c2nc(-c3ccccc3)nc(-c3ccc(-n4c5ccc6sc7ccccc7c6c5c5ccc6ccccc6c54)cc3)n2)cc1]

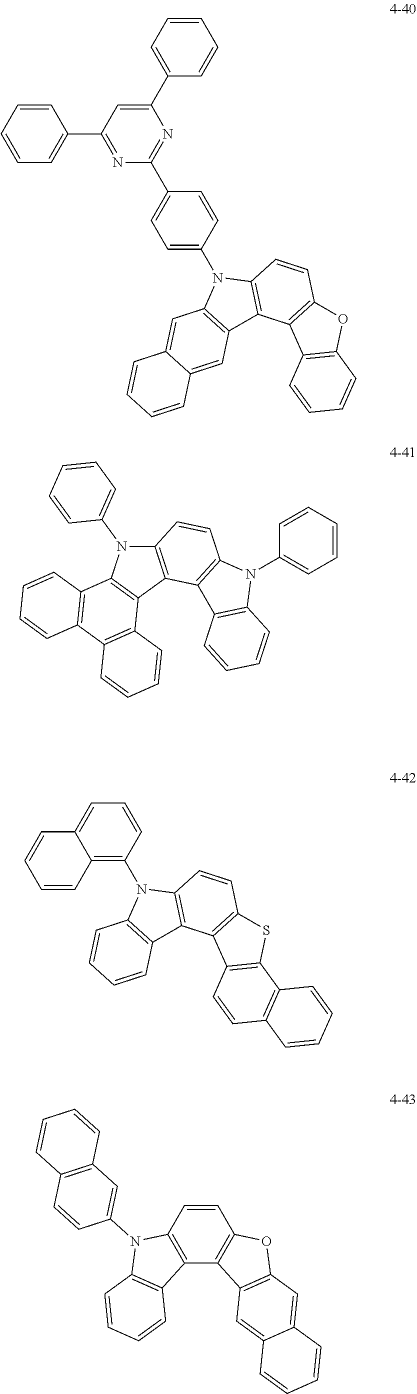 [SMILES: c1ccc(-c2cc(-c3ccccc3)nc(-c3ccc(-n4c5cc6ccccc6cc5c5c6c(ccc54)oc4ccccc46)cc3)n2)cc1 ; c1ccc(-n2c3ccccc3c3c4c5c6ccccc6c6ccccc6c5n(-c5ccccc5)c4ccc32)cc1 ; c1ccc2c(-n3c4ccccc4c4c5c(ccc43)sc3c4ccccc4ccc35)cccc2c1 ; c1ccc2cc(-n3c4ccccc4c4c5c(ccc43)oc3cc4ccccc4cc35)ccc2c1]

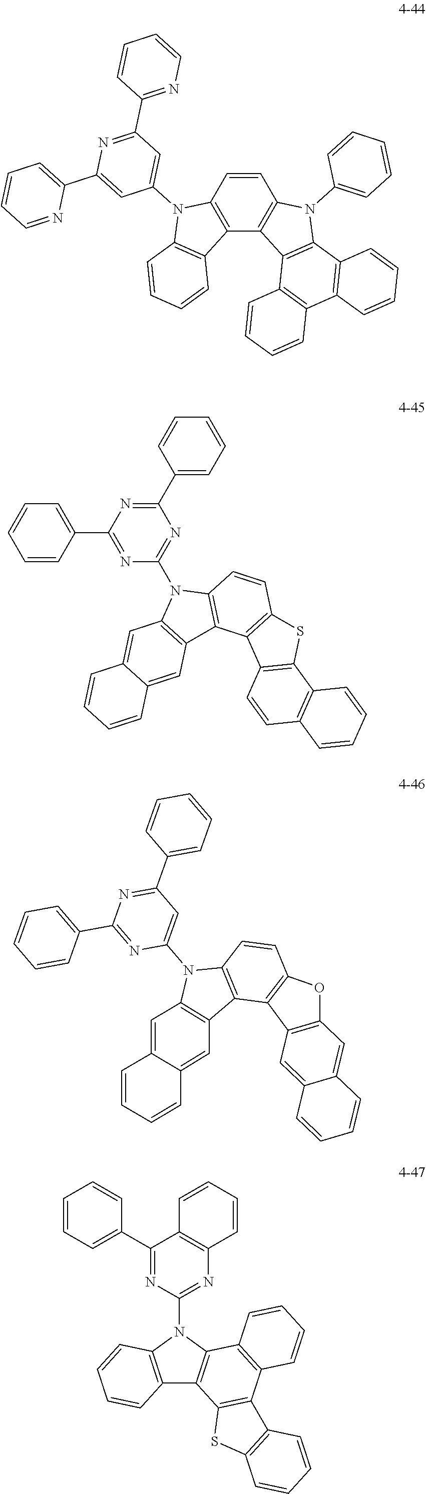 [SMILES: c1ccc(-n2c3ccc4c(c5ccccc5n4-c4cc(-c5ccccn5)nc(-c5ccccn5)c4)c3c3c4ccccc4c4ccccc4c32)cc1 ; c1ccc(-c2nc(-c3ccccc3)nc(-n3c4cc5ccccc5cc4c4c5c(ccc43)sc3c4ccccc4ccc35)n2)cc1 ; c1ccc(-c2cc(-n3c4cc5ccccc5cc4c4c5c(ccc43)oc3cc4ccccc4cc35)nc(-c3ccccc3)n2)cc1 ; c1ccc(-c2nc(-n3c4ccccc4c4c5sc6ccccc6c5c5ccccc5c43)nc3ccccc23)cc1]

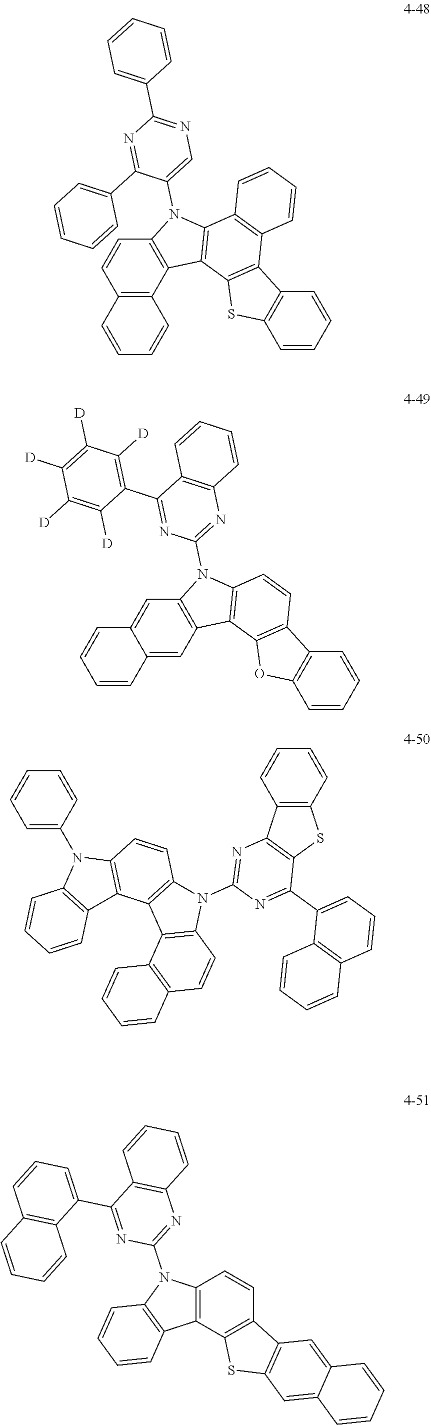

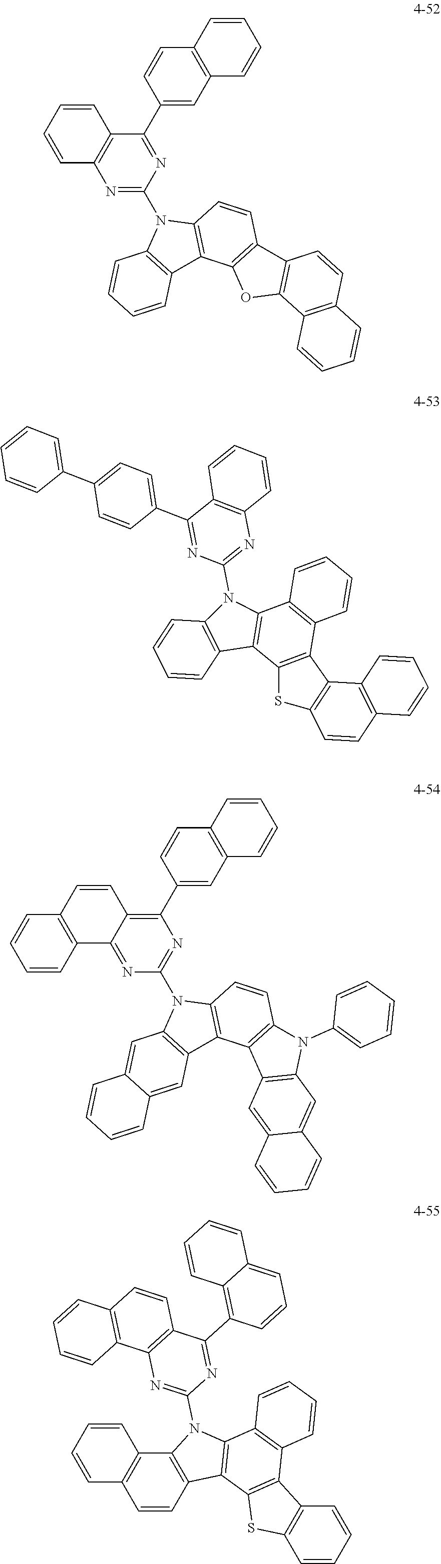 [SMILES: c1ccc2cc(-c3nc(-n4c5ccccc5c5c6oc7c8ccccc8ccc7c6ccc54)nc4ccccc34)ccc2c1 ; c1ccc(-c2ccc(-c3nc(-n4c5ccccc5c5c6sc7ccc8ccccc8c7c6c6ccccc6c54)nc4ccccc34)cc2)cc1 ; c1ccc(-n2c3cc4ccccc4cc3c3c4c5cc6ccccc6cc5n(-c5nc(-c6ccc7ccccc7c6)c6ccc7ccccc7c6n5)c4ccc32)cc1 ; c1ccc2c(-c3nc(-n4c5c6ccccc6ccc5c5c6sc7ccccc7c6c6ccccc6c54)nc4c3ccc3ccccc34)cccc2c1]

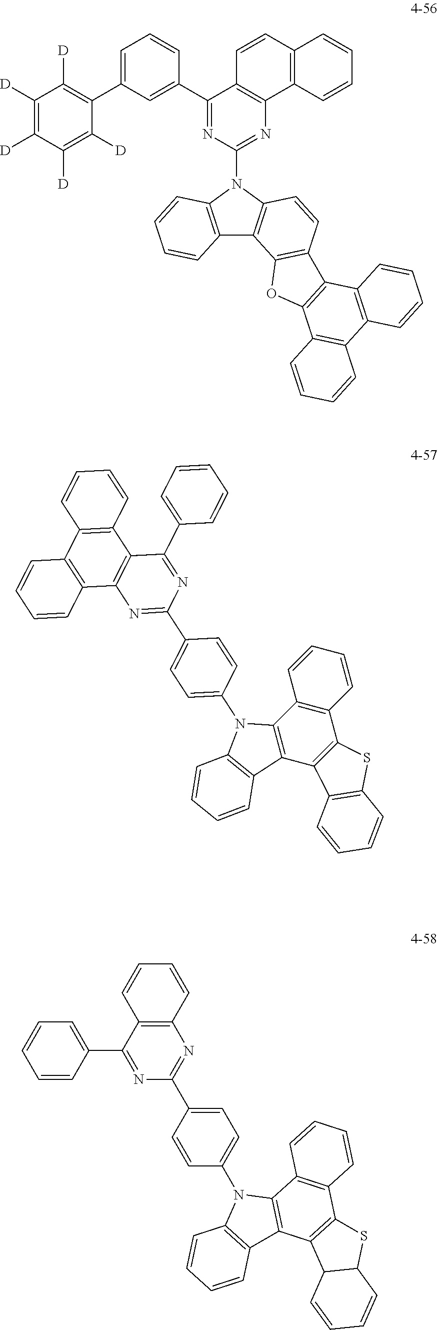 [SMILES: [2H]c1c([2H])c([2H])c(-c2cccc(-c3nc(-n4c5ccccc5c5c6oc7c8ccccc8c8ccccc8c7c6ccc54)nc4c3ccc3ccccc34)c2)c([2H])c1[2H] ; c1ccc(-c2nc(-c3ccc(-n4c5ccccc5c5c6c7ccccc7sc6c6ccccc6c54)cc3)nc3c4ccccc4c4ccccc4c23)cc1 ; C1=CC2Sc3c(c4c5ccccc5n(-c5ccc(-c6nc(-c7ccccc7)c7ccccc7n6)cc5)c4c4ccccc34)C2C=C1]

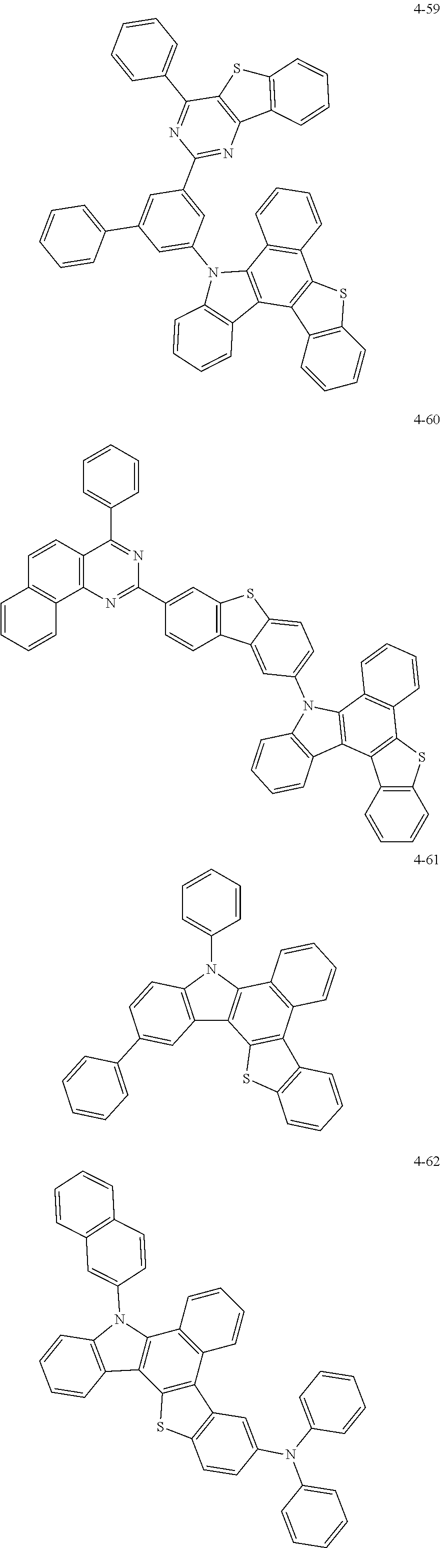 [SMILES: c1ccc(-c2cc(-c3nc(-c4ccccc4)c4sc5ccccc5c4n3)cc(-n3c4ccccc4c4c5c6ccccc6sc5c5ccccc5c43)c2)cc1 ; c1ccc(-c2nc(-c3ccc4c(c3)sc3ccc(-n5c6ccccc6c6c7c8ccccc8sc7c7ccccc7c65)cc34)nc3c2ccc2ccccc23)cc1 ; c1ccc(-c2ccc3c(c2)c2c4sc5ccccc5c4c4ccccc4c2n3-c2ccccc2)cc1 ; c1ccc(N(c2ccccc2)c2ccc3sc4c(c3c2)c2ccccc2c2c4c3ccccc3n2-c2ccc3ccccc3c2)cc1]

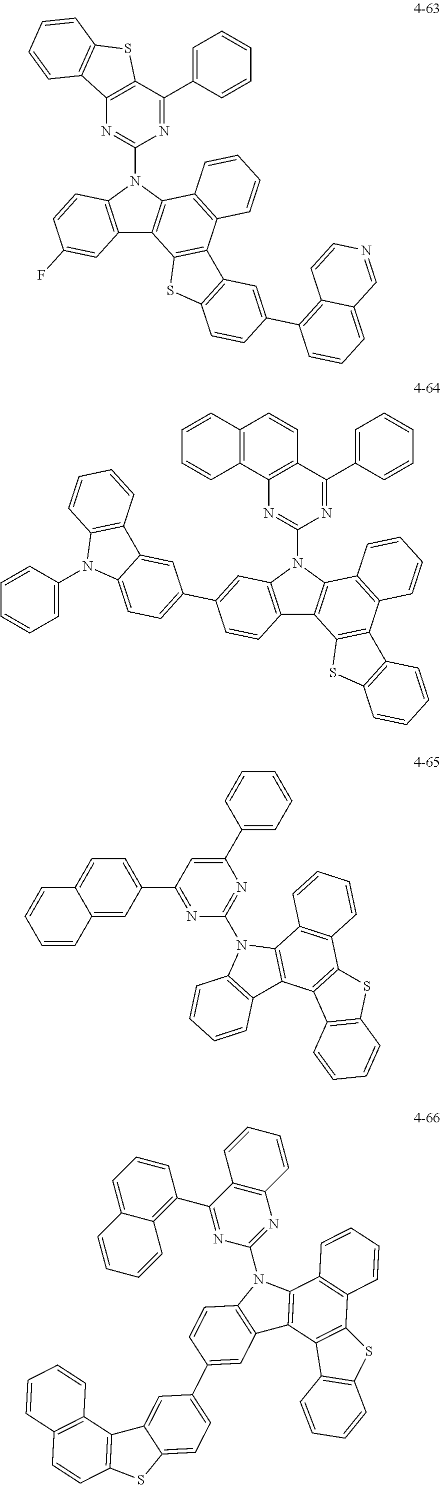 [SMILES: Fc1ccc2c(c1)c1c3sc4ccc(-c5cccc6cnccc56)cc4c3c3ccccc3c1n2-c1nc(-c2ccccc2)c2sc3ccccc3c2n1 ; c1ccc(-c2nc(-n3c4cc(-c5ccc6c(c5)c5ccccc5n6-c5ccccc5)ccc4c4c5sc6ccccc6c5c5ccccc5c43)nc3c2ccc2ccccc23)cc1 ; c1ccc(-c2cc(-c3ccc4ccccc4c3)nc(-n3c4ccccc4c4c5c6ccccc6sc5c5ccccc5c43)n2)cc1 ; c1ccc2c(-c3nc(-n4c5ccc(-c6ccc7sc8ccc9ccccc9c8c7c6)cc5c5c6c7ccccc7sc6c6ccccc6c54)nc4ccccc34)cccc2c1]

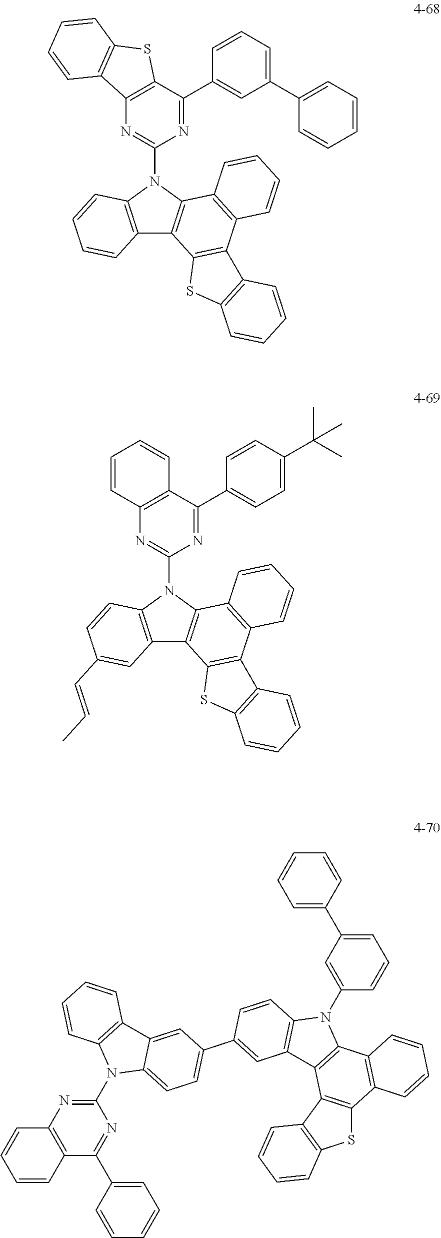 [SMILES: c1ccc(-c2cccc(-c3nc(-n4c5ccccc5c5c6sc7ccccc7c6c6ccccc6c54)nc4c3sc3ccccc34)c2)cc1 ; C/C=C/c1ccc2c(c1)c1c3sc4ccccc4c3c3ccccc3c1n2-c1nc(-c2ccc(C(C)(C)C)cc2)c2ccccc2n1 ; c1ccc(-c2cccc(-n3c4ccc(-c5ccc6c(c5)c5ccccc5n6-c5nc(-c6ccccc6)c6ccccc6n5)cc4c4c5c6ccccc6sc5c5ccccc5c43)c2)cc1]

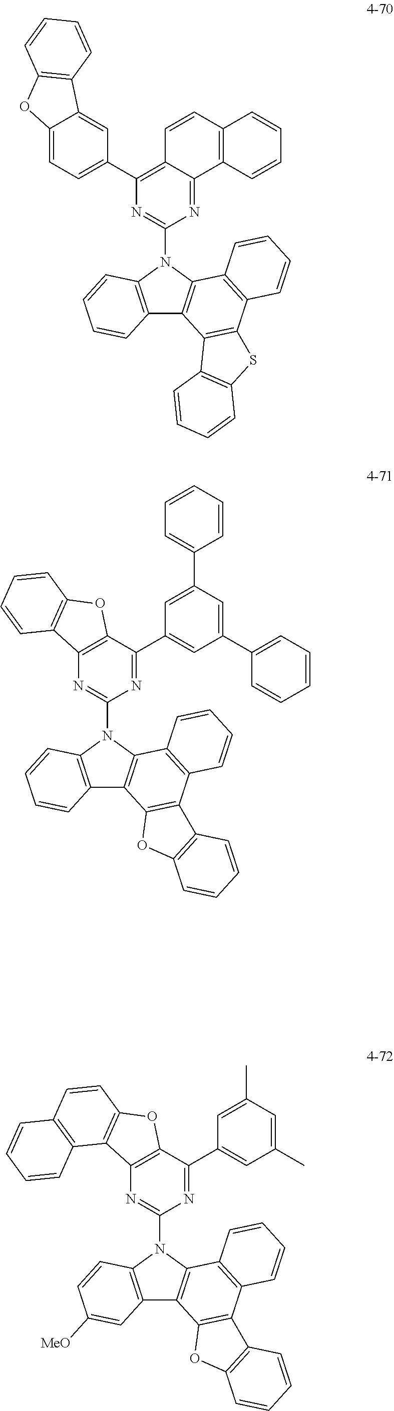 [SMILES: c1ccc2c(c1)ccc1c(-c3ccc4oc5ccccc5c4c3)nc(-n3c4ccccc4c4c5c6ccccc6sc5c5ccccc5c43)nc12 ; c1ccc(-c2cc(-c3ccccc3)cc(-c3nc(-n4c5ccccc5c5c6oc7ccccc7c6c6ccccc6c54)nc4c3oc3ccccc34)c2)cc1 ; COc1ccc2c(c1)c1c3oc4ccccc4c3c3ccccc3c1n2-c1nc(-c2cc(C)cc(C)c2)c2oc3ccc4ccccc4c3c2n1]

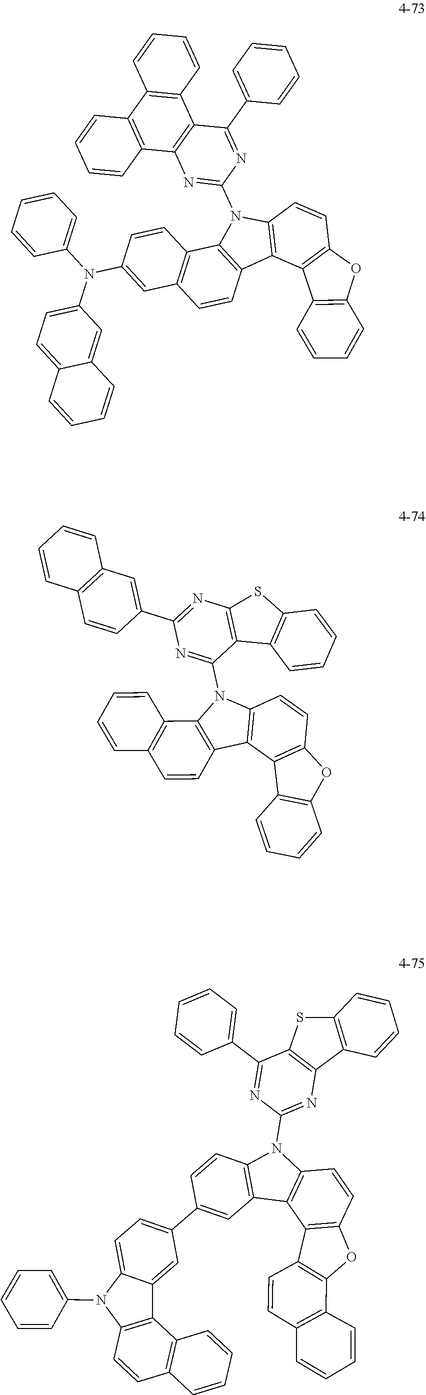 [SMILES: c1ccc(-c2nc(-n3c4ccc5oc6ccccc6c5c4c4ccc5cc(N(c6ccccc6)c6ccc7ccccc7c6)ccc5c43)nc3c4ccccc4c4ccccc4c23)cc1 ; c1ccc2cc(-c3nc(-n4c5ccc6oc7ccccc7c6c5c5ccc6ccccc6c54)c4c(n3)sc3ccccc34)ccc2c1 ; c1ccc(-c2nc(-n3c4ccc(-c5ccc6c(c5)c5c7ccccc7ccc5n6-c5ccccc5)cc4c4c5c(ccc43)oc3c4ccccc4ccc35)nc3c2sc2ccccc23)cc1]

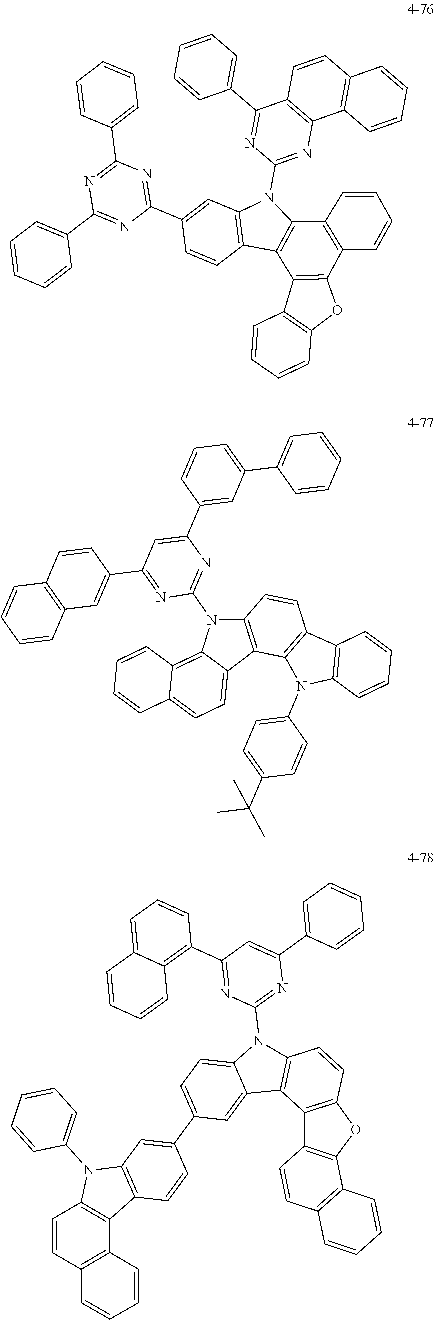 [SMILES: c1ccc(-c2nc(-c3ccccc3)nc(-c3ccc4c5c6c7ccccc7oc6c6ccccc6c5n(-c5nc(-c6ccccc6)c6ccc7ccccc7c6n5)c4c3)n2)cc1 ; CC(C)(C)c1ccc(-n2c3ccccc3c3ccc4c(c5ccc6ccccc6c5n4-c4nc(-c5cccc(-c6ccccc6)c5)cc(-c5ccc6ccccc6c5)n4)c32)cc1 ; c1ccc(-c2cc(-c3cccc4ccccc34)nc(-n3c4ccc(-c5ccc6c7c8ccccc8ccc7n(-c7ccccc7)c6c5)cc4c4c5c(ccc43)oc3c4ccccc4ccc35)n2)cc1]

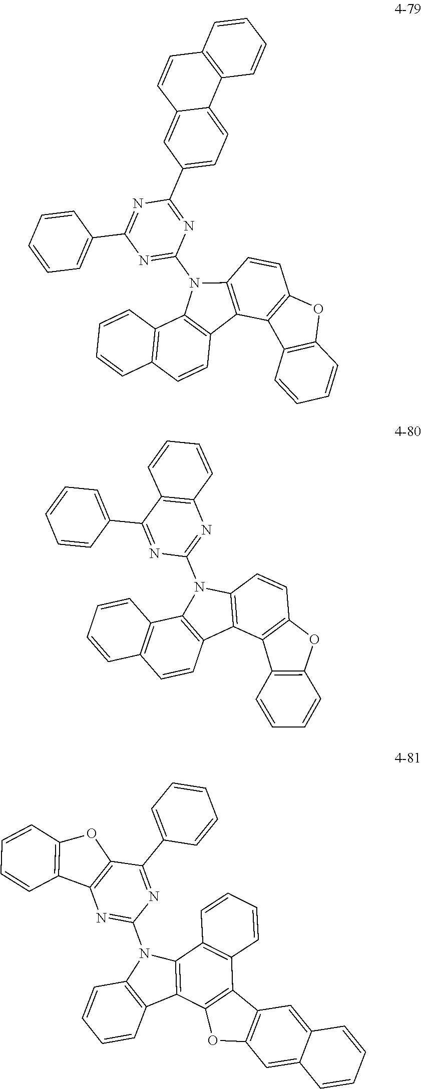 [SMILES: c1ccc(-c2nc(-c3ccc4c(ccc5ccccc54)c3)nc(-n3c4ccc5oc6ccccc6c5c4c4ccc5ccccc5c43)n2)cc1 ; c1ccc(-c2nc(-n3c4ccc5oc6ccccc6c5c4c4ccc5ccccc5c43)nc3ccccc23)cc1 ; c1ccc(-c2nc(-n3c4ccccc4c4c5oc6cc7ccccc7cc6c5c5ccccc5c43)nc3c2oc2ccccc23)cc1]

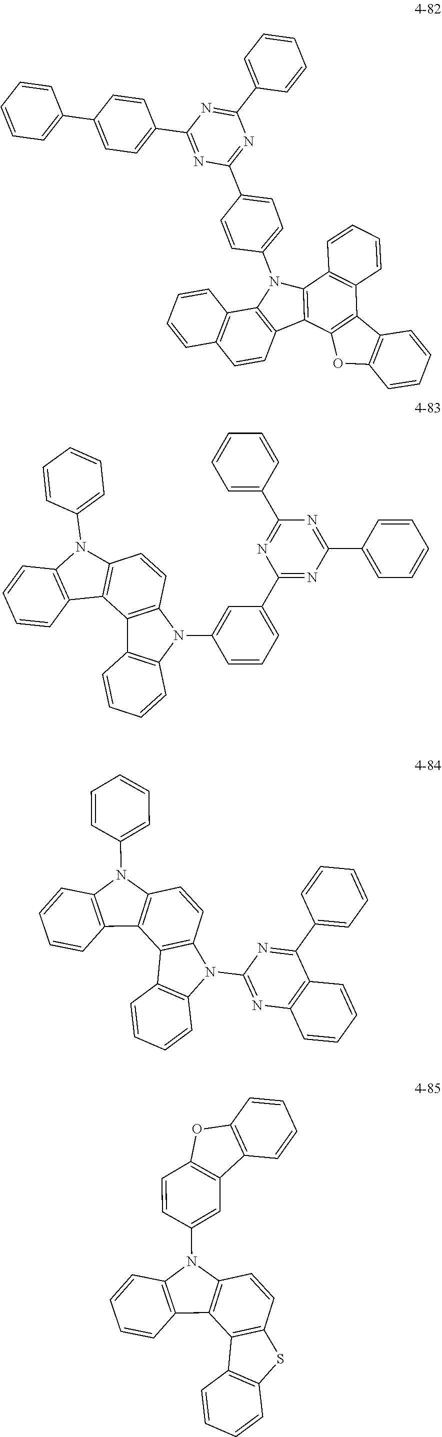 [SMILES: c1ccc(-c2ccc(-c3nc(-c4ccccc4)nc(-c4ccc(-n5c6c7ccccc7ccc6c6c7oc8ccccc8c7c7ccccc7c65)cc4)n3)cc2)cc1 ; c1ccc(-c2nc(-c3ccccc3)nc(-c3cccc(-n4c5ccccc5c5c6c7ccccc7n(-c7ccccc7)c6ccc54)c3)n2)cc1 ; c1ccc(-c2nc(-n3c4ccccc4c4c5c6ccccc6n(-c6ccccc6)c5ccc43)nc3ccccc23)cc1 ; c1ccc2c(c1)oc1ccc(-n3c4ccccc4c4c5c(ccc43)sc3ccccc35)cc12]

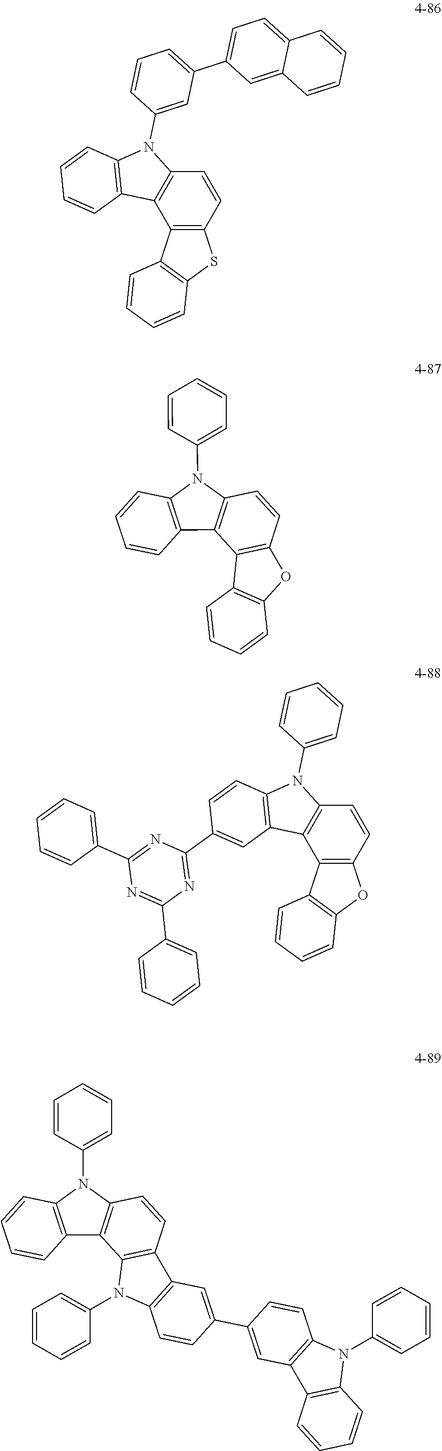 [SMILES: c1cc(-c2ccc3ccccc3c2)cc(-n2c3ccccc3c3c4c(ccc32)sc2ccccc24)c1 ; c1ccc(-n2c3ccccc3c3c4c(ccc32)oc2ccccc24)cc1 ; c1ccc(-c2nc(-c3ccccc3)nc(-c3ccc4c(c3)c3c5c(ccc3n4-c3ccccc3)oc3ccccc35)n2)cc1 ; c1ccc(-n2c3ccccc3c3cc(-c4ccc5c(c4)c4ccc6c(c7ccccc7n6-c6ccccc6)c4n5-c4ccccc4)ccc32)cc1]

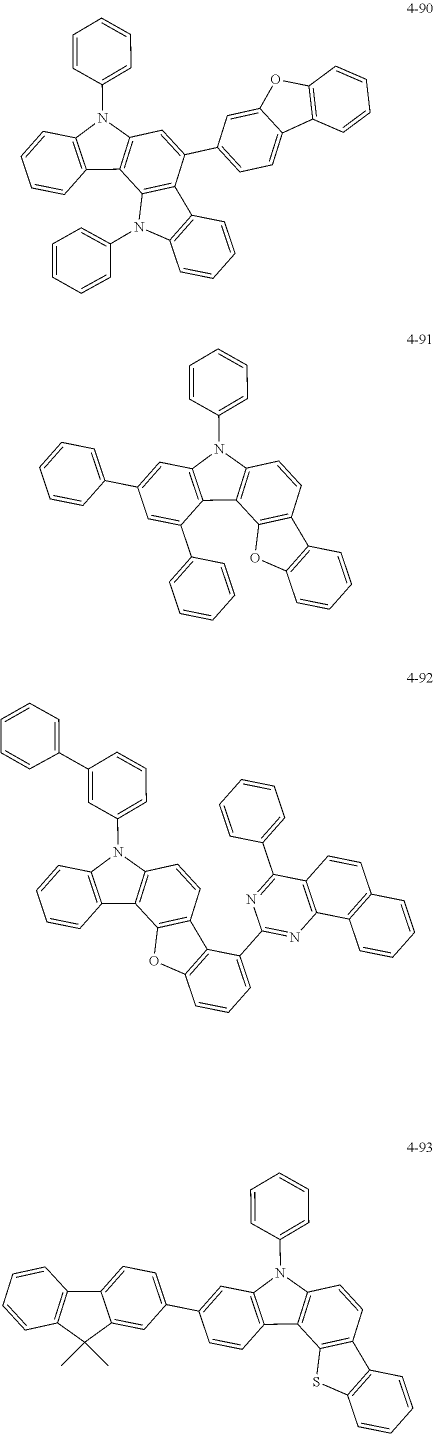 [SMILES: c1ccc(-n2c3ccccc3c3c2cc(-c2ccc4c(c2)oc2ccccc24)c2c4ccccc4n(-c4ccccc4)c23)cc1 ; c1ccc(-c2cc(-c3ccccc3)c3c4c5oc6ccccc6c5ccc4n(-c4ccccc4)c3c2)cc1 ; c1ccc(-c2cccc(-n3c4ccccc4c4c5oc6cccc(-c7nc(-c8ccccc8)c8ccc9ccccc9c8n7)c6c5ccc43)c2)cc1 ; CC1(C)c2ccccc2-c2ccc(-c3ccc4c5c6sc7ccccc7c6ccc5n(-c5ccccc5)c4c3)cc21]

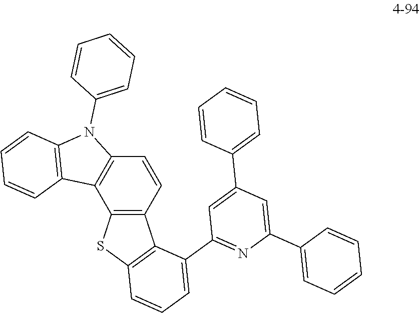 [SMILES: c1ccc(-c2cc(-c3ccccc3)nc(-c3cccc4sc5c(ccc6c5c5ccccc5n6-c5ccccc5)c34)c2)cc1]

11. The organic electronic element of claim 1, comprising at least one hole transport band layer between the anode and the emitting layer, wherein the hole transport band layer comprises a hole transport layer, an emitting auxiliary layer, or both, wherein the hole transport band layer comprises a compound represented by Formula 1.

12. The organic electronic element of claim 1, further comprising a light efficiency enhancing layer formed on at least one surface of the anode and the cathode, the surface being opposite to the organic material layer.

13. The organic electronic element of claim 1, wherein the organic material layer comprises 2 or more stacks comprising a hole transport layer, an emitting layer, and an electron transport layer sequentially formed on the anode.

14. The organic electronic element of claim 1, wherein the organic material layer further comprises a charge generating layer formed between 2 or more stacks.

15. An electronic device comprising: a display device comprising the organic electronic element of claim 1; and a control unit for driving the display device.

16. The electronic device of claim 15, wherein the organic electronic element is at least one of an OLED, an organic solar cell, an organic photo conductor (OPC), an organic transistor (organic TFT), and an element for monochromic or white illumination.
